(19) United States
(12) Patent Application Publication
(10) Pub. No.: US 2024/0023432 A1
(43) Pub. Date: Jan. 18, 2024

(54) HETEROCYCLIC COMPOUND, ORGANIC LIGHT-EMITTING DEVICE COMPRISING SAME, MANUFACTURING METHOD THEREFOR, AND COMPOSITION FOR ORGANIC LAYER

(71) Applicant: LT MATERIALS CO., LTD., Yongin-si, Gyeonggi-do (KR)

(72) Inventors: Seong Jong PARK, Yongin-si (KR); Nam Jin LEE, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/034,441

(22) PCT Filed: Nov. 5, 2021
(86) PCT No.: PCT/KR2021/016014

§ 371 Date: Apr. 28, 2023

(30) Foreign Application Priority Data
Dec. 17, 2020 (KR) 10-2020-0177117

(51) Int. Cl. H10K 85/60; C07D 307/77; C09K 11/06; C07D 307/91

(52) U.S. Cl. CPC H10K 85/636; C07D 307/77; H10K 50/15; H10K 85/633; C07D 307/91; C09K 11/06

(57) ABSTRACT

The present specification relates to a heterocyclic compound represented by Formula 1, an organic light emitting device comprising the same, a manufacturing method thereof, and a composition for an organic layer.

### TECHNICAL FIELD

The present application claims the benefit of priority based on Korean Patent Application No. 10-2020-0177117 filed on Dec. 17, 2020, the entire contents of which are incorporated herein by reference.

The present invention relates to a heterocyclic compound, an organic light emitting device comprising the same, a method for preparing the same, and a composition for organic layer.

### BACKGROUND ART

An organic light emitting device is a kind of self-emitting display device, which has the advantage of having a wide viewing angle, excellent contrast, and also fast response speed.

The organic light emitting device has a structure in which an organic thin film is disposed between two electrodes. When a voltage is applied to the organic light emitting device having such structure, electrons and holes injected from the two electrodes are combined in the organic thin film to form a pair, and then emit light while disappearing. The organic thin film may be composed of a single layer or multiple layers, as required.

The material of the organic thin film may have a light emitting function, if necessary. For example, as a material for the organic thin film, a compound capable of forming a light emitting layer by itself alone may be used, or a compound capable of serving as a host or dopant of a host-dopant-based light emitting layer may also be used. In addition, as a material for the organic thin film, a compound capable of performing the roles of hole injection, hole transport, electron blocking, hole blocking, electron transport, electron injection, and the like may be used.

In order to improve the performance, lifetime or efficiency of an organic light emitting device, there is a continuous demand for the development of materials for the organic thin film.

### PRIOR ART DOCUMENT

### Patent Document

U.S. Pat. No. 4,356,429

### DISCLOSURE

### Technical Problem

It is an object of the present invention to provide a heterocyclic compound, an organic light emitting device comprising the same, a manufacturing method thereof, and a composition for organic layer.

### Technical Solution

The present invention provides a heterocyclic compound represented by Formula 1 below:

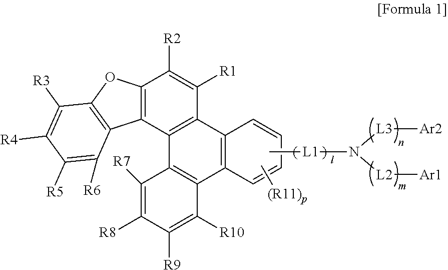

wherein,
Ar1 and Ar2 are the same as or different from each other, and are each independently a substituted or unsubstituted C6 to C60 aryl group; or a substituted or unsubstituted C2 to C60 heteroaryl group,
R1 to R11 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
L1 to L3 are the same as or different from each other, and are each independently a direct bond; a substituted or unsubstituted C6 to C60 arylene group; or a substituted or unsubstituted C2 to C60 heteroarylene group,
l, m, and n are the same as or different from each other, and each independently are an integer from 0 to 5, when l is 2 or more, each L1 is the same as or different from each other, when m is 2 or more, each L2 is the same as or different from each other, and when n is 2 or more, each L3 is the same as or different from each other,
p is an integer from 1 to 3, and when p is 2 or more, each R11 is the same as or different from each other.

In addition, the present invention provides an organic light emitting device comprising a first electrode; a second electrode provided to face the first electrode; and one or more organic layers provided between the first electrode and the second electrode, wherein at least one of the one or more organic layers comprises a heterocyclic compound represented by Formula 1 above.

In addition, the present invention provides an organic light emitting device, wherein the one or more organic layers comprises a hole transport layer, and the hole transport layer comprises the heterocyclic compound.

In addition, the present invention provides an organic light emitting device, wherein the one or more organic layer comprises an electron blocking layer, and the electron blocking layer comprises the heterocyclic compound.

In addition, the present invention provides a composition for organic layer of an organic light emitting device comprising the heterocyclic compound represented by Formula 1 above.

In addition, the present invention provides a method for manufacturing an organic light emitting device comprising the steps of preparing a substrate; forming a first electrode on the substrate; forming one or more organic layers on the first electrode; and forming a second electrode on the one or more organic layers, wherein the step of forming the one or more organic layers comprises a step of forming one or more organic layers using the composition for organic layer of the organic light emitting device.

### Advantageous Effect

The heterocyclic compound according to an embodiment of the present application may be used as a material for an organic layer of an organic light emitting device. The heterocyclic compound may be used as a material for a hole injection layer, a hole transport layer, an electron blocking layer, a light emitting layer, an electron transport layer, a hole blocking layer, an electron injection layer, a charge generating layer and the like in an organic light emitting device. In particular, the heterocyclic compound represented by Formula 1 may be used as a material for a hole transport layer or a material for an electron blocking layer of an organic light emitting device.

Specifically, the heterocyclic compound represented by Formula 1 may be used alone or in combination with other compounds as a material for a hole transport layer or a material for an electron blocking layer. If the compound represented by Formula 1 is used for an organic layer, the operating voltage of the organic light emitting device is lowered, the luminous efficiency is improved, and the lifetime characteristics of the device can be improved due to the thermal stability of the compound.

### DESCRIPTION OF DRAWING

FIGS. 1 to 4 are views schematically showing a stacked structure of an organic light emitting device according to an embodiment of the present invention, respectively.

### BEST MODE

Hereinafter, the present application will be described in detail.

In the present specification, the term “substituted” means that a hydrogen atom bonded to a carbon atom of a compound is replaced with another substituent, the position to be substituted is not limited as long as it is a position where a hydrogen atom may be substituted, that is, it is a position that can be substituted by a substituent, and when substituted by two or more substituents, two or more substituents may be the same or different from each other.

In the present specification, the term “substituted or unsubstituted” means that it is substituted or unsubstituted by one or more substituents selected from the group consisting of C1 to C60 straight or branched chain alkyl; C2 to C60 straight or branched alkenyl; C2 to C60 straight or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or means that it is substituted or unsubstituted with a substituent formed by connecting two or more substituents selected from the above-exemplified substituents.

In the present specification, the halogen may be fluorine, chlorine, bromine, or iodine.

In the present specification, the alkyl group comprises a straight or branched chain having 1 to 60 carbon atoms, and may be further substituted by other substituents. The number of carbon atoms in the alkyl group may be 1 to 60, specifically 1 to 40, more specifically, 1 to 20. Specific examples of the alkyl group may be, but is not limited to, a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like.

In the present specification, the alkenyl group comprises a straight or branched chain having 2 to 60 carbon atoms, and may be further substituted by other substituents. The carbon number of the alkenyl group may be 2 to 60, specifically 2 to 40, more specifically 2 to 20. Specific examples of the alkenyl group may be, but is not limited to, a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like.

In the present specification, the alkynyl group comprises a straight or branched chain having 2 to 60 carbon atoms, and may be further substituted by other substituents. The carbon number of the alkynyl group may be 2 to 60, specifically 2 to 40, more specifically 2 to 20.

In the present specification, the alkoxy group may be a straight-chain, a branched-chain or a cyclic chain. Although the number of carbon atoms in the alkoxy group is not particularly limited, it is preferable that the number of carbon atoms is 1-20. Specific examples of the alkoxy group may be, but is not limited to, methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like.

In the present specification, the cycloalkyl group comprises a monocyclic or polycyclic ring having 3 to 60 carbon atoms, and may be further substituted by other substituents. In this case, the polycyclic ring refers to a group formed by directly connecting or condensing a cycloalkyl group with another cyclic group. In this case, the other cyclic group may be a cycloalkyl group, but may be a different type of cyclic group, for example, a heterocycloalkyl group, an aryl group, a heteroaryl group, or the like. The number of carbon atoms in the cycloalkyl group may be 3 to 60, specifically 3 to 40, more specifically 5 to 20. Specific examples of the cycloalkyl group may be, but is not limited to, a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like.

In the present specification, the heterocycloalkyl group comprises a monocyclic or polycyclic ring containing 0, S, Se, N or Si as a hetero atom and having 2 to 60 carbon atoms, and may be further substituted by other substituents. In this case, the polycyclic ring refers to a group formed by directly connecting or condensing a heterocycloalkyl group with another cyclic group. In this case, the other cyclic group may be a heterocycloalkyl group, but may be a different type of cyclic group, for example, a cycloalkyl group, an aryl group, a heteroaryl group, or the like. The number of carbon atoms in the heterocycloalkyl group may be 2 to 60, specifically 2 to 40, more specifically 3 to 20.

In the present specification, the aryl group comprises a monocyclic or polycyclic ring having 6 to 60 carbon atoms, and may be further substituted by other substituents. In this case, the polycyclic ring means a group formed by directly connecting or condensing an aryl group with another cyclic group. In this case, the other cyclic group may be an aryl group, but may be a different type of cyclic group, for example, a cycloalkyl group, a heterocycloalkyl group, a heteroaryl group, or the like. The aryl group comprises a spiro group. The number of carbon atoms in the aryl group may be 6 to 60, specifically 6 to 40, more specifically 6 to 25. Specific examples of the aryl group may be, but is not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenylenyl group, a pyrenyl group, a tetracenyl group, a pentacenyl group, a fluorenyl group, an indenyl group, an acenaphthyl group, a benzofluorenyl group, a spirobifluorenyl group, a 2,3-dihydro-1H-indenyl group, condensed ring groups thereof and the like.

In the present specification, the phosphine oxide group is represented by —P(═O)R101R102, wherein R101 and R102 are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specifically, it may be substituted with an aryl group, and the above-described examples may be applied to the aryl group. For example, the phosphine oxide group is, but is not limited to, a diphenyl phosphine oxide group, dinaphthyl phosphine oxide group, or the like.

In the present specification, the silyl group contains Si and is a substituent formed by directly connecting the Si atoms as a radical, and is represented by —SiR₁₀₄R₁₀₅R₁₀₆, wherein R₁₀₄ to R₁₀₅ are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specific examples of the silyl group may be, but is not limited to, a trimethylsilyl group, a triethylsilyl group, a t-butyldimethylsilyl group, vinyldimethylsilyl group, a propyldimethylsilyl group, a triphenylsilyl group, a diphenylsilyl group, a phenylsilyl group and the like.

In the present specification, the fluorenyl group may be substituted, and adjacent substituents may be combined with each other to form a ring.

If the fluorenyl group is substituted, it may be

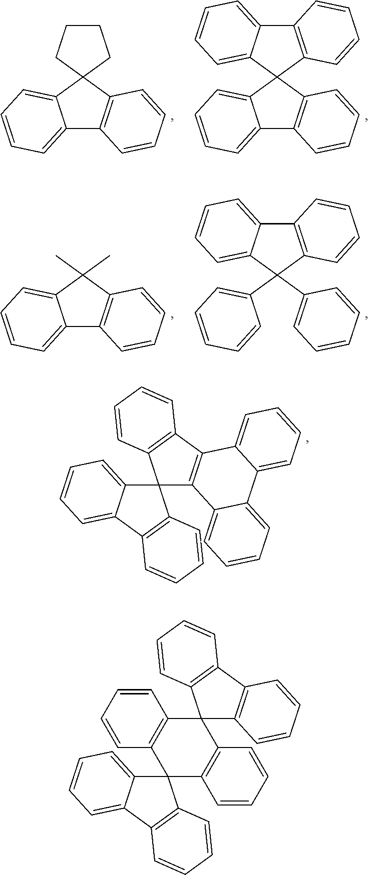

and the like, but is not limited thereto.

In the present specification, the heteroaryl group comprises a monocyclic or polycyclic ring containing S, O, Se, N, or Si as a hetero atom and having 2 to 60 carbon atoms, and may be further substituted by other substituents. In this case, the polycyclic ring refers to a group formed by directly connecting or condensing a heteroaryl group with another cyclic group. In this case, the other cyclic group may be a heteroaryl group, but may be a different type of cyclic group, for example, a cycloalkyl group, a heterocycloalkyl group, an aryl group, or the like. The number of carbon atoms in the heteroaryl group may be 2 to 60, specifically 2 to 40, more specifically 3 to 25. Specific examples of the heteroaryl group may be, but is not limited to, a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, tetrazinyl group, a quinolyl group, an isoquinolyl group, a quinazolinyl group, an isoquinazolinyl group, a quinazolylyl group, a naphthyridyl group, an acridinyl group, a phenanthridinyl group, an imidazopyridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilolyl group, spirobi(dibenzosilolyl), a dihydrophenazinyl group, a phenoxazinyl group, a phenanthridyl group, an imidazopyridinyl group, a thienyl group, an indolo[2,3-a] carbazolyl group, an indolo[2,3-b] carbazolyl group, an indolinyl group, a 10,11-dihydro-dibenzo[b,f]azepinyl group, a 9,10-dihydroacridinyl group, a phenanthrazinyl group, a phenothiazinyl group, phthalazinyl group, a naphthylidinyl group, a phenanthrolinyl group, a benzo[c][1,2,5]thiadiazolyl group, 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms of the amine group is not particularly limited, but is preferably 1 to 30. Specific examples of the amine group may be, but is not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like.

In the present specification, the arylene group means that the aryl group has two bonding positions, that is, a divalent group. These are the same as those described for the aryl group described above, except that each of them is a divalent group. In addition, the heteroarylene group means that the heteroaryl group has two bonding positions, that is, a divalent group. These are the same as those described for the heteroaryl group described above, except that each of them is a divalent group.

In the present specification, an “adjacent” group may mean a substituent substituted at the atom directly linked to the atom substituted by that substituent, a substituent that is sterically closest to that substituent, or another substituent substituted at the atom substituted by that substituent. For example, two substituents substituted at an ortho position in a benzene ring and two substituents substituted at the same carbon in an aliphatic ring may be interpreted as “adjacent” groups to each other.

In the present invention, “when a substituent is not indicated in the structure of the formula or the compound” means that a hydrogen atom is bonded to a carbon atom. However, since deuterium (²H) is an isotope of hydrogen, some hydrogen atoms may be deuterium.

In one embodiment of the present invention, “when a substituent is not indicated in the structure of the formula or the compound” may mean that all positions that may be substituted by the substituent are hydrogen or deuterium. That is, in the case of deuterium, some hydrogen atoms as an isotope of hydrogen may be isotopes of deuterium. In this case, the content of deuterium may be 0% to 100%.

In one embodiment of the present invention, in the case of “when a substituent is not indicated in the structure of the formula or the compound”, if deuterium is not explicitly excluded, except that “the content of deuterium is 0%”, “the content of hydrogen is 100%”, “all substituents are hydrogen” etc., thus hydrogen and deuterium may be used in admixture in the compound.

In one embodiment of the present invention, deuterium is one of the isotopes of hydrogen, and is an element having as an atomic nucleus a deuteron consisting of one proton and one neutron, and can be expressed as hydrogen-2, and the element symbol can also be written as D or ²H.

In one embodiment of the present invention, an isotope means an atom with the same atomic number (Z) but a different mass number (A), and can also be interpreted as an element that has the same number of protons but the different number of neutrons.

In one embodiment of the present invention, the meaning of content T % of a specific substituent may be defined as T2/T1×100=T %, wherein T1 is defined as the total number of substituents that the basic compound can have, and T2 is defined as the number of specific substituents substituted among them.

That is, in one example, the 20% content of deuterium in the phenyl group represented by

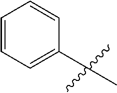 [SMILES: *c1ccccc1]

may mean that the total number of substituents that the phenyl group can have is 5 (T1 in the formula), and the number of deuterium among them is 1 (T2 in the formula). That is, in the phenyl group, that the content of deuterium is 20% may be represented by Structural Formulas below:

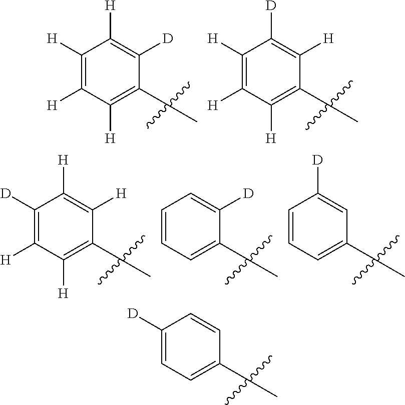

Also, in one embodiment of the present invention, in the case of “a phenyl group having a deuterium content of 0%”, it means a phenyl group that does not contain a deuterium atom, i.e., has 5 hydrogen atoms.

In the present invention, the content of deuterium in the heterocyclic compound represented by Formula 1 may be 0 to 100%, more preferably 30 to 100%.

In the present invention, C6 to C60 aromatic hydrocarbon ring means a compound containing an aromatic ring consisting of C6 to C60 carbons and hydrogen, and for example, may be, but is not limited to, benzene, biphenyl, terphenyl, triphenylene, naphthalene, anthracene, phenalene, phenanthrene, fluorene, pyrene, chrysene, perylene, azulene and the like, and comprises all of the aromatic hydrocarbon ring compounds known in the art as satisfying the carbon number described above.

The present invention provides a heterocyclic compound represented by the following Formula 1:

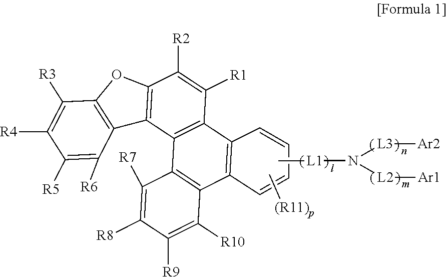

wherein,
Ar1 and Ar2 are the same as or different from each other, and are each independently a substituted or unsubstituted C6 to C60 aryl group; or a substituted or unsubstituted C2 to C60 heteroaryl group,
R1 to R11 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
L1 to L3 are the same as or different from each other, and are each independently a direct bond; a substituted or unsubstituted C6 to C60 arylene group; or a substituted or unsubstituted C2 to C60 heteroarylene group,
l, m and n are the same as or different from each other, and each independently are an integer of 0 to 5, and when l is 2 or more, each L1 is the same as or different from each other, and when m is 2 or more, each L2 is the same as or different from each other, and when n is 2 or more, each L3 is the same as or different from each other,
p is an integer from 1 to 3, and when p is 2 or more, each R11 is the same as or different from each other.

The aliphatic or aromatic hydrocarbon ring or hetero ring that may be formed by the adjacent groups may be structures exemplified by the aforementioned cycloalkyl group, the cycloheteroalkyl group, the aryl group, and the heteroaryl group, except that it is not a monovalent group.

In one embodiment of the present invention, Ar1 and Ar2 may be the same as or different from each other, and may each independently be a substituted or unsubstituted C6 to C30 aryl group; or a substituted or unsubstituted C2 to C30 heteroaryl group.

In other embodiment of the present invention, Ar1 and Ar2 may be the same as or different from each other, and may each independently be a substituted or unsubstituted C6 to C20 aryl group; or a substituted or unsubstituted C2 to C20 heteroaryl group.

In other embodiment of the present invention, Ar1 and Ar2 may be the same as or different from each other, and may each independently be a substituted or unsubstituted phenyl group, a naphthyl group, a fluorenyl group; or a substituted or unsubstituted dibenzofuranyl group.

In one embodiment of the present invention, R1 to R11 may be the same as or different from each other, and may each independently be hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In other embodiment of the present invention, R1 to R11 may be the same as or different from each other, and may each independently be hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In other embodiment of the present invention, R1 to R11 may be the same as or different from each other, and may each independently be hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In other embodiment of the present invention, R1 to R11 may be the same as or different from each other, and may each independently be hydrogen; deuterium; a substituted or unsubstituted C6 to C20 aryl group; or a substituted or unsubstituted C2 to C20 heteroaryl group.

In other embodiment of the present invention, R1 to R11 may be the same as or different from each other, and each independently may be hydrogen; or deuterium.

In one embodiment of the present invention, L1 to L3 above may be the same as or different from each other, and may each independently be a direct bond; a substituted or unsubstituted C6 to C60 arylene group; or a substituted or unsubstituted C2 to C60 heteroarylene group.

In other embodiment of the present invention, L1 to L3 may be the same as or different from each other, and may each independently be a direct bond; a substituted or unsubstituted C6 to C30 arylene group; or a substituted or unsubstituted C2 to C30 heteroarylene group.

In other embodiment of the present invention, L1 to L3 may be the same as or different from each other, and may each independently be a direct bond; a substituted or unsubstituted C6 to C20 arylene group; or a substituted or unsubstituted C2 to C20 heteroarylene group.

In other embodiment of the present invention, L1 to L3 may be the same as or different from each other, and may each independently be a direct bond; or a substituted or unsubstituted phenylene group.

In one embodiment of the present invention, l, m, and n may be the same as or different from each other, and may each independently be an integer of 1 to 3, and when l is 2 or more, each L1 may be the same as or different from each other, and when m is 2 or more, each L2 may be the same as or different from each other, and when n is 2 or more, each L3 may be the same as or different from each other.

In other embodiment of the present invention, l, m, and n may be the same as or different from each other, and may each independently be an integer of 1 to 2, when l is 2, each L1 may be the same as or different from each other, when m is 2, each L2 may be the same or different from each other, and when n is 2, each L3 may be the same as or different from each other.

In other embodiment of the present invention, the ‘substitution’ of Ar1, Ar2, R1 to R11, and L1 to L3 may each independently be made by one or more substituents selected from the group consisting of deuterium; a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In other embodiment of the present invention, the ‘substitution’ of Ar1, Ar2, R1 to R11, and L1 to L3 may each independently be made by one or more substituents selected from the group consisting of deuterium; a C1 to C10 alkyl group; a C6 to C30 aryl group; a C2 to C30 heteroaryl group.

In other embodiment of the present invention, the ‘substitution’ of Ar1, Ar2, R1 to R11, and L1 to L3 may each independently be made by one or more substituents selected from the group consisting of deuterium; a C1 to C5 alkyl group; a C6 to C20 aryl group; and a C2 to C20 heteroaryl group.

In other embodiment of the present invention, the ‘substitution’ of Ar1, Ar2, R1 to R11, and L1 to L3 may each independently be made by one or more substituents selected from the group consisting of deuterium, a methyl group, an ethyl group, a straight or branched propyl group, a straight or branched butyl group, a straight or branched pentyl group, a phenyl group, a naphthyl group, a pyridinyl group, an anthracenyl group, a carbazolyl group, a dibenzothiophenyl group, a dibenzofuranyl group, and a phenanthrenyl group.

In other embodiment of the present invention, the ‘substitution’ of Ar1, Ar2, R1 to R11, and L1 to L3 may each independently be made by deuterium, a methyl group, an ethyl group, a straight or branched propyl group, a straight or branched butyl group, and a straight or branched pentyl group.

In one embodiment of the present invention, Formula 1 may be a heterocyclic compound represented by Formula 1-1 or Formula 1-2 below:

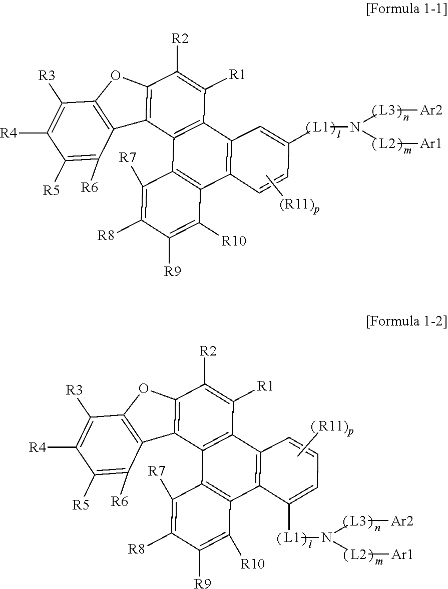

wherein Ar1, Ar2, R1 to R11, L1 to L3, l, m, n, and p are the same as defined in Formula 1 above.

In one embodiment of the present invention, the heterocyclic compound represented by Formula 1 may be at least one selected from the compounds below:

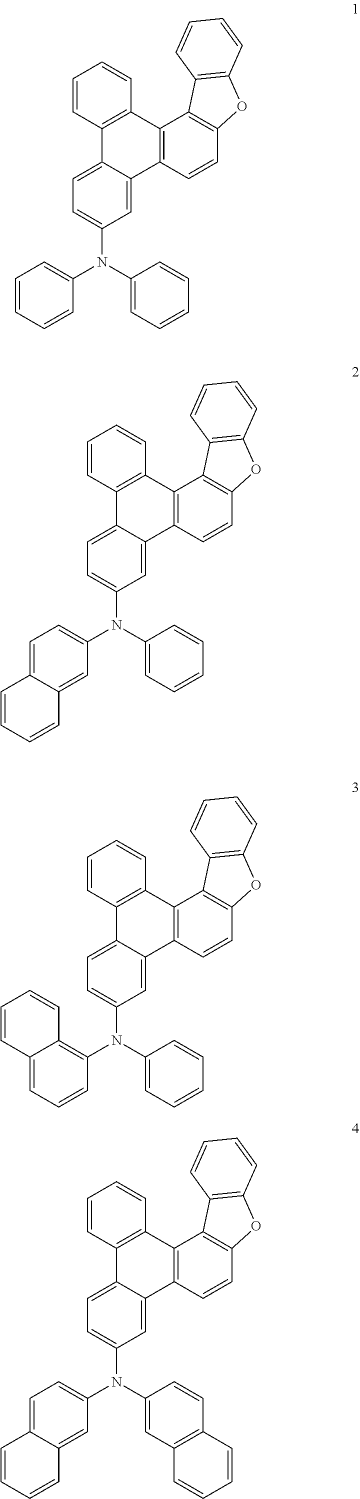

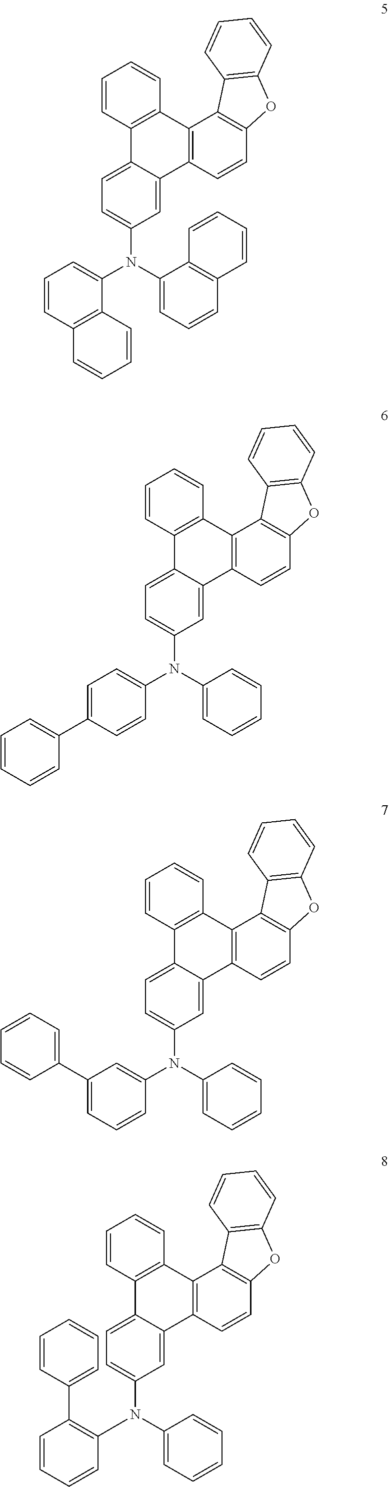

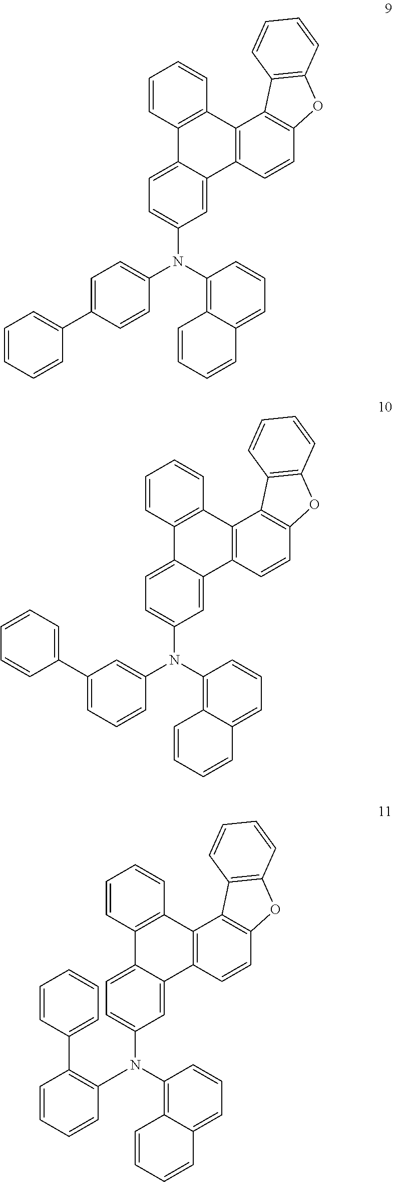 [SMILES: c1ccc(-c2ccc(N(c3ccc4c5ccccc5c5c(ccc6oc7ccccc7c65)c4c3)c3cccc4ccccc34)cc2)cc1 ; c1ccc(-c2cccc(N(c3ccc4c5ccccc5c5c(ccc6oc7ccccc7c65)c4c3)c3cccc4ccccc34)c2)cc1 ; c1ccc(-c2ccccc2N(c2ccc3c4ccccc4c4c(ccc5oc6ccccc6c54)c3c2)c2cccc3ccccc23)cc1]

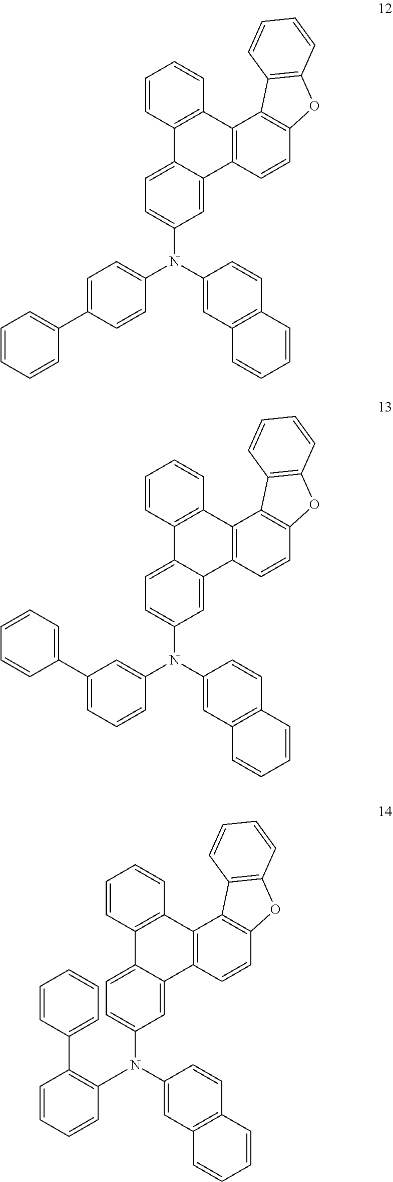

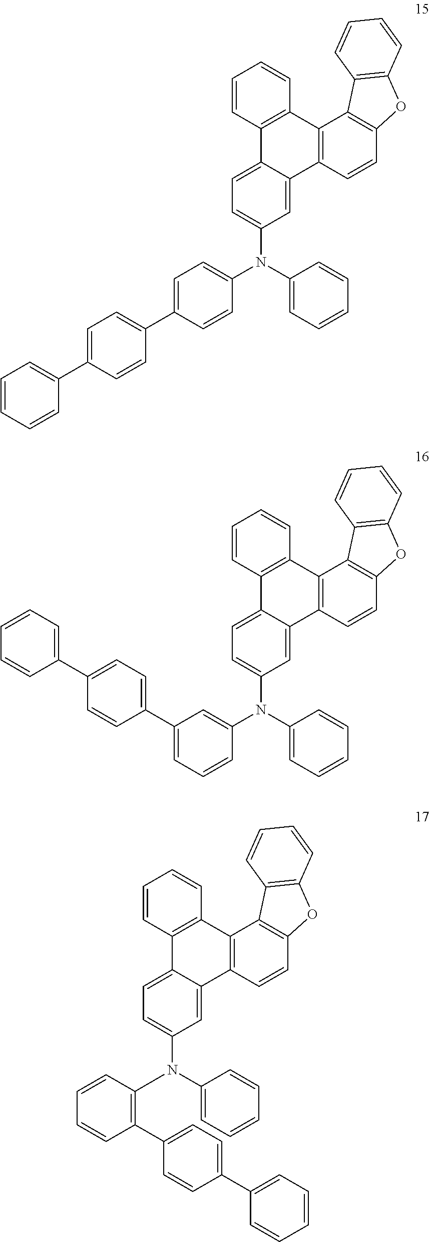 [SMILES: c1ccc(-c2ccc(-c3ccc(N(c4ccccc4)c4ccc5c6ccccc6c6c(ccc7oc8ccccc8c76)c5c4)cc3)cc2)cc1 ; c1ccc(-c2ccc(-c3cccc(N(c4ccccc4)c4ccc5c6ccccc6c6c(ccc7oc8ccccc8c76)c5c4)c3)cc2)cc1 ; c1ccc(-c2ccc(-c3ccccc3N(c3ccccc3)c3ccc4c5ccccc5c5c(ccc6oc7ccccc7c65)c4c3)cc2)cc1]

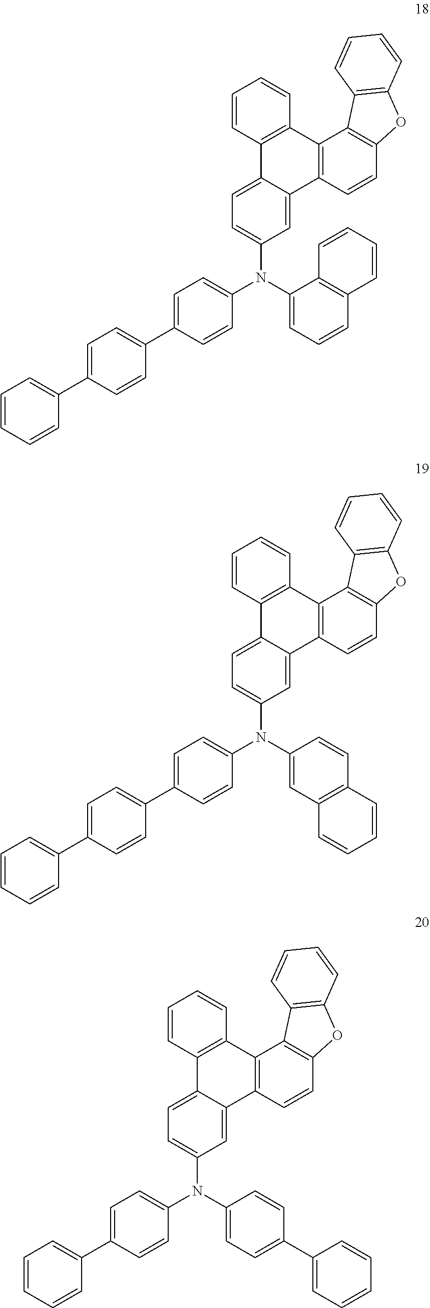 [SMILES: c1ccc(-c2ccc(-c3ccc(N(c4ccc5c6ccccc6c6c(ccc7oc8ccccc8c76)c5c4)c4cccc5ccccc45)cc3)cc2)cc1 ; c1ccc(-c2ccc(-c3ccc(N(c4ccc5ccccc5c4)c4ccc5c6ccccc6c6c(ccc7oc8ccccc8c76)c5c4)cc3)cc2)cc1 ; c1ccc(-c2ccc(N(c3ccc(-c4ccccc4)cc3)c3ccc4c5ccccc5c5c(ccc6oc7ccccc7c65)c4c3)cc2)cc1]

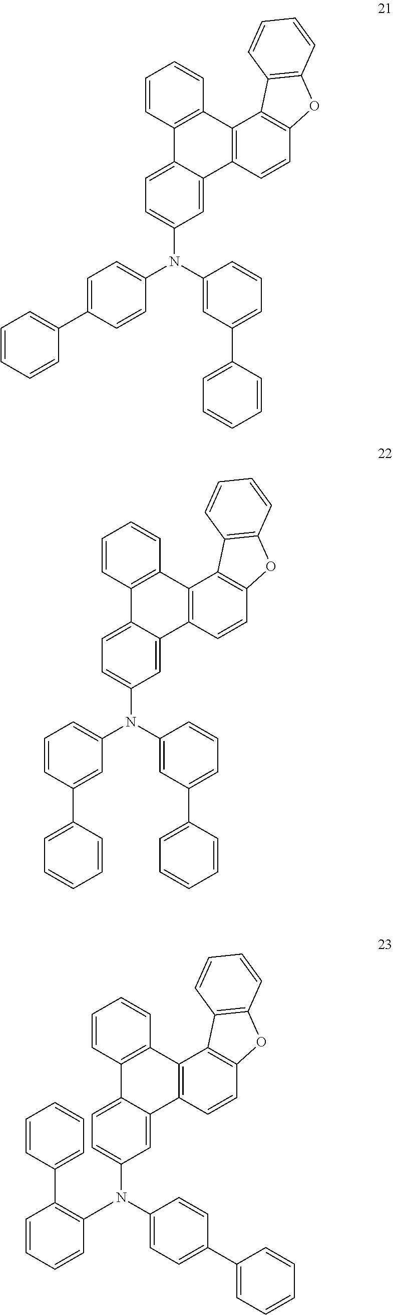 [SMILES: c1ccc(-c2ccc(N(c3cccc(-c4ccccc4)c3)c3ccc4c5ccccc5c5c(ccc6oc7ccccc7c65)c4c3)cc2)cc1 ; c1ccc(-c2cccc(N(c3cccc(-c4ccccc4)c3)c3ccc4c5ccccc5c5c(ccc6oc7ccccc7c65)c4c3)c2)cc1 ; c1ccc(-c2ccc(N(c3ccc4c5ccccc5c5c(ccc6oc7ccccc7c65)c4c3)c3ccccc3-c3ccccc3)cc2)cc1]

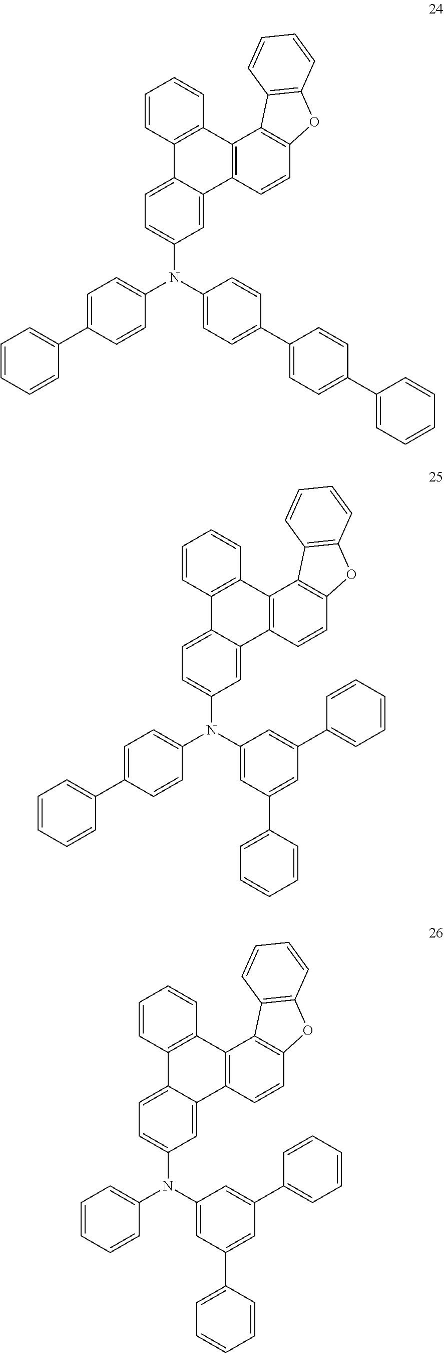 [SMILES: c1ccc(-c2ccc(-c3ccc(N(c4ccc(-c5ccccc5)cc4)c4ccc5c6ccccc6c6c(ccc7oc8ccccc8c76)c5c4)cc3)cc2)cc1 ; c1ccc(-c2ccc(N(c3cc(-c4ccccc4)cc(-c4ccccc4)c3)c3ccc4c5ccccc5c5c(ccc6oc7ccccc7c65)c4c3)cc2)cc1 ; c1ccc(-c2cc(-c3ccccc3)cc(N(c3ccccc3)c3ccc4c5ccccc5c5c(ccc6oc7ccccc7c65)c4c3)c2)cc1]

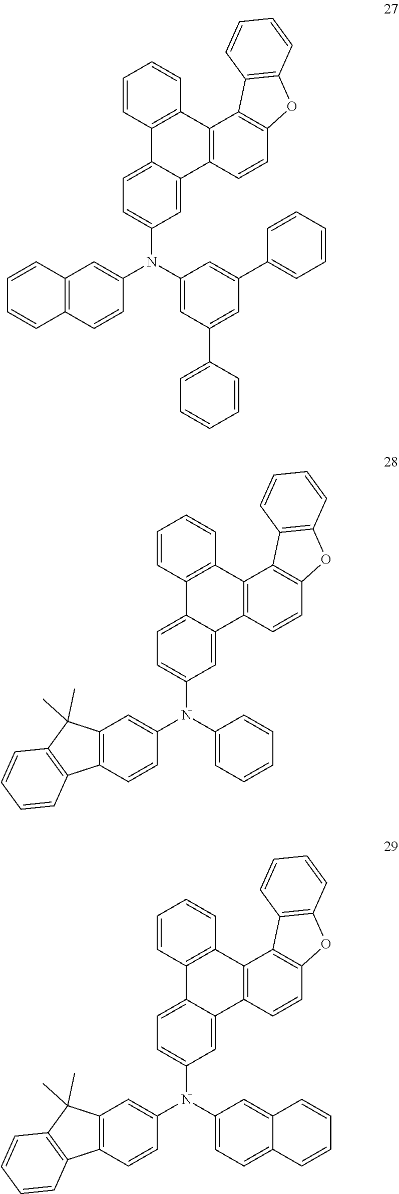 [SMILES: c1ccc(-c2cc(-c3ccccc3)cc(N(c3ccc4ccccc4c3)c3ccc4c5ccccc5c5c(ccc6oc7ccccc7c65)c4c3)c2)cc1 ; CC1(C)c2ccccc2-c2ccc(N(c3ccccc3)c3ccc4c5ccccc5c5c(ccc6oc7ccccc7c65)c4c3)cc21 ; CC1(C)c2ccccc2-c2ccc(N(c3ccc4ccccc4c3)c3ccc4c5ccccc5c5c(ccc6oc7ccccc7c65)c4c3)cc21]

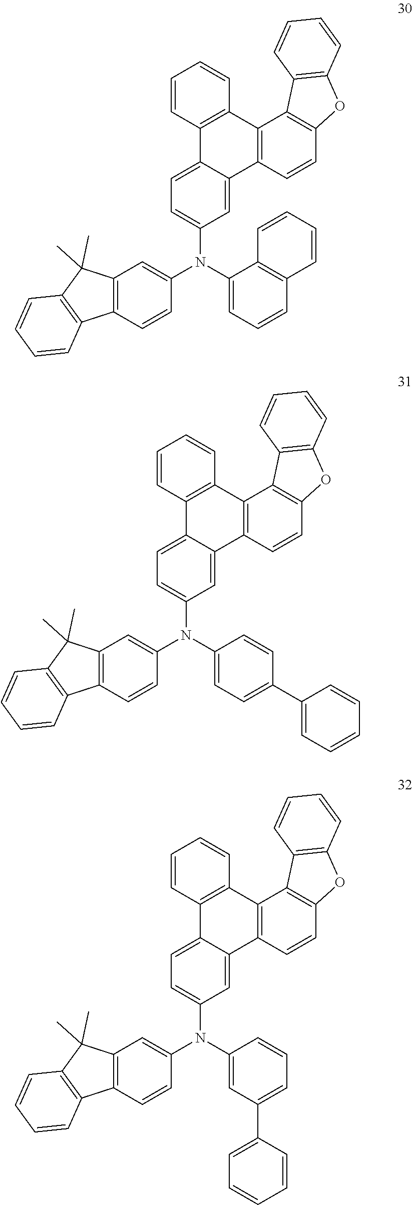

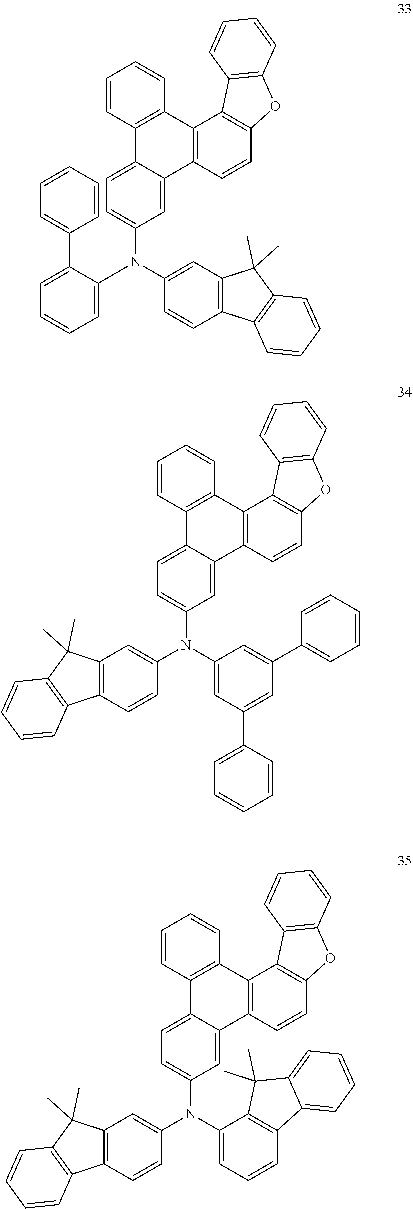

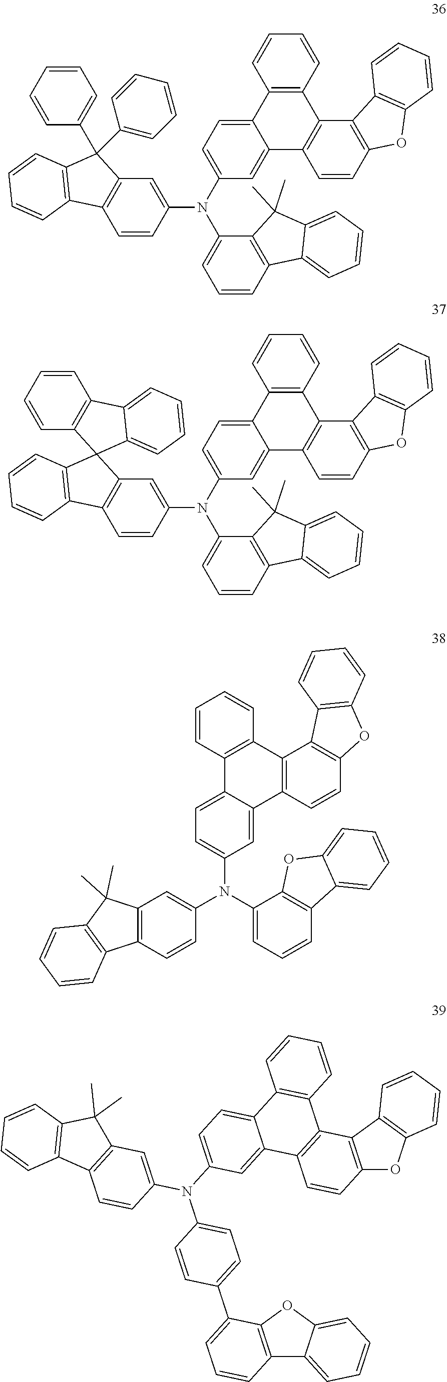

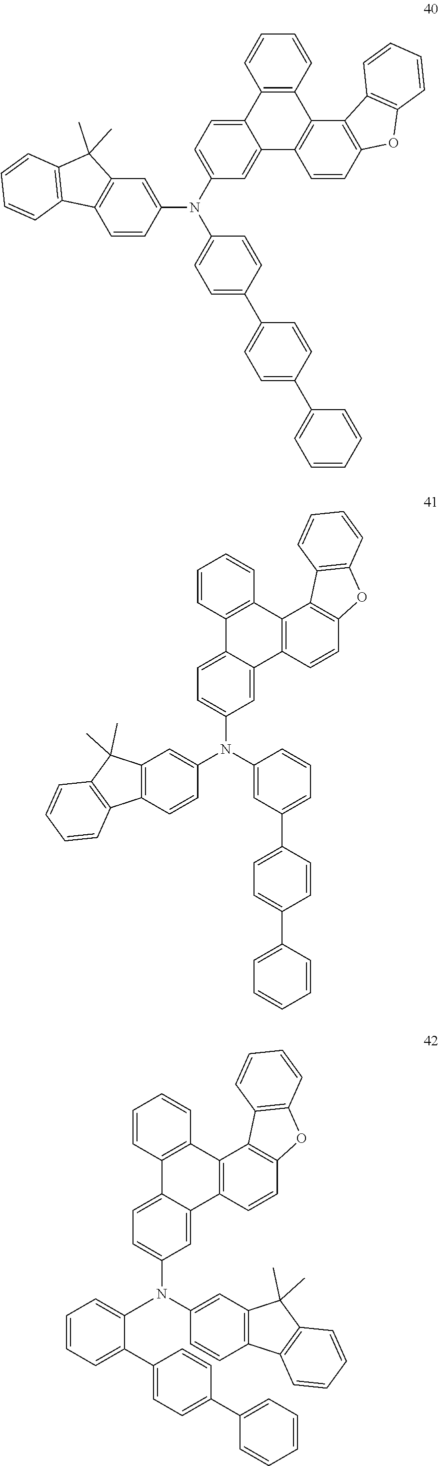

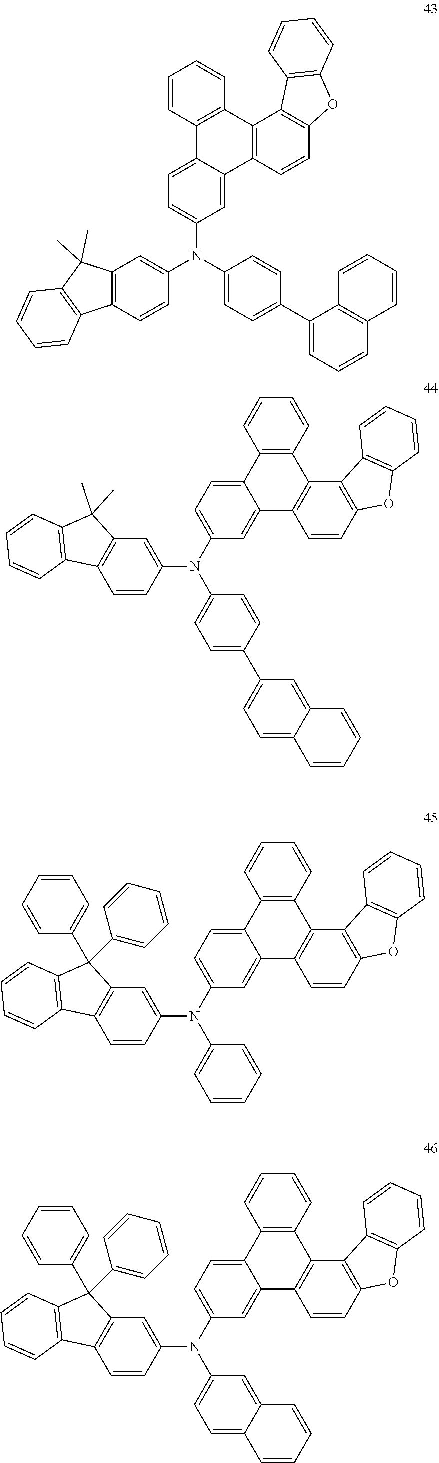

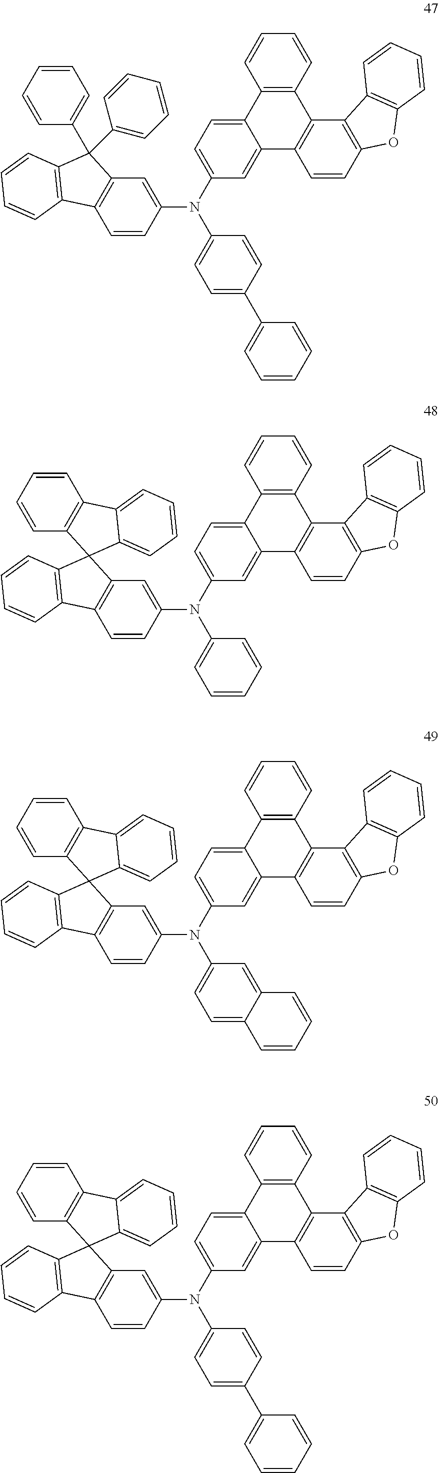 [SMILES: c1ccc(-c2ccc(N(c3ccc4c(c3)C(c3ccccc3)(c3ccccc3)c3ccccc3-4)c3ccc4c5ccccc5c5c(ccc6oc7ccccc7c65)c4c3)cc2)cc1 ; c1ccc(N(c2ccc3c(c2)C2(c4ccccc4-c4ccccc42)c2ccccc2-3)c2ccc3c4ccccc4c4c(ccc5oc6ccccc6c54)c3c2)cc1 ; c1ccc2c(c1)-c1ccccc1C21c2ccccc2-c2ccc(N(c3ccc4ccccc4c3)c3ccc4c5ccccc5c5c(ccc6oc7ccccc7c65)c4c3)cc21 ; c1ccc(-c2ccc(N(c3ccc4c(c3)C3(c5ccccc5-c5ccccc53)c3ccccc3-4)c3ccc4c5ccccc5c5c(ccc6oc7ccccc7c65)c4c3)cc2)cc1]

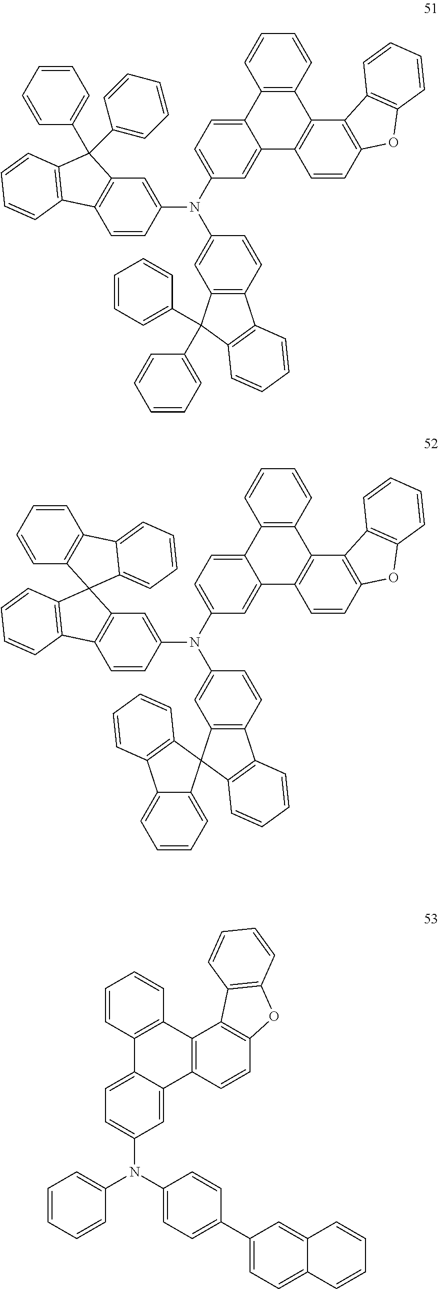 [SMILES: c1ccc(C2(c3ccccc3)c3ccccc3-c3ccc(N(c4ccc5c(c4)C(c4ccccc4)(c4ccccc4)c4ccccc4-5)c4ccc5c6ccccc6c6c(ccc7oc8ccccc8c76)c5c4)cc32)cc1 ; c1ccc2c(c1)-c1ccccc1C21c2ccccc2-c2ccc(N(c3ccc4c(c3)C3(c5ccccc5-c5ccccc53)c3ccccc3-4)c3ccc4c5ccccc5c5c(ccc6oc7ccccc7c65)c4c3)cc21 ; c1ccc(N(c2ccc(-c3ccc4ccccc4c3)cc2)c2ccc3c4ccccc4c4c(ccc5oc6ccccc6c54)c3c2)cc1]

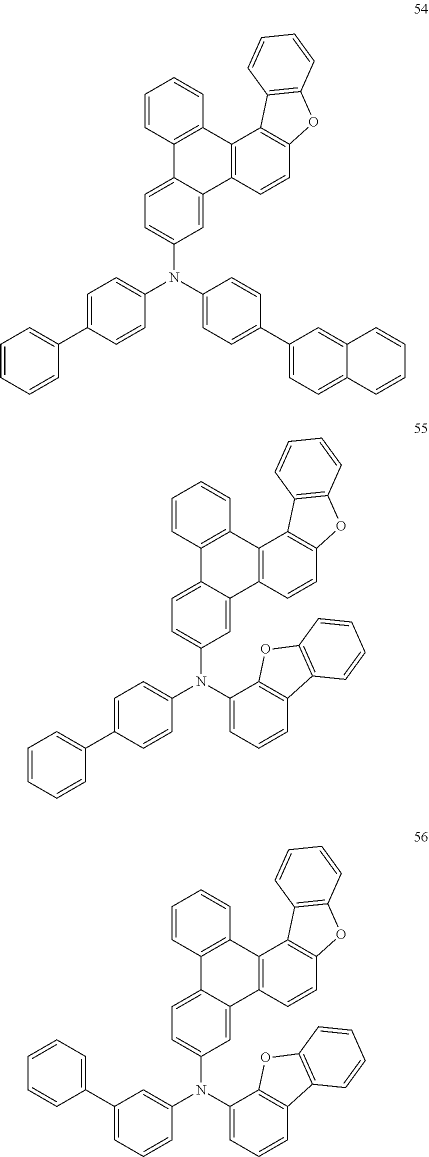

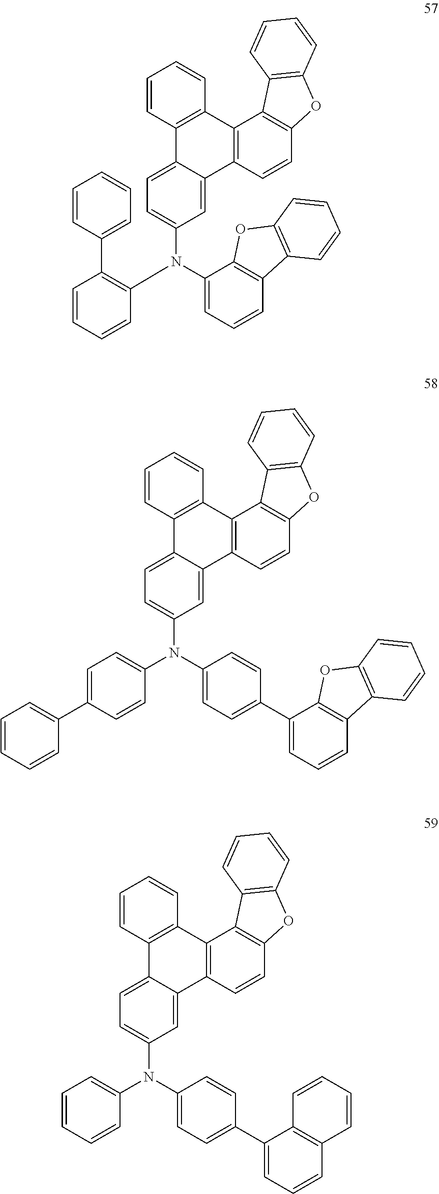 [SMILES: c1ccc(-c2ccccc2N(c2ccc3c4ccccc4c4c(ccc5oc6ccccc6c54)c3c2)c2cccc3c2oc2ccccc23)cc1 ; c1ccc(-c2ccc(N(c3ccc(-c4cccc5c4oc4ccccc45)cc3)c3ccc4c5ccccc5c5c(ccc6oc7ccccc7c65)c4c3)cc2)cc1 ; c1ccc(N(c2ccc(-c3cccc4ccccc34)cc2)c2ccc3c4ccccc4c4c(ccc5oc6ccccc6c54)c3c2)cc1]

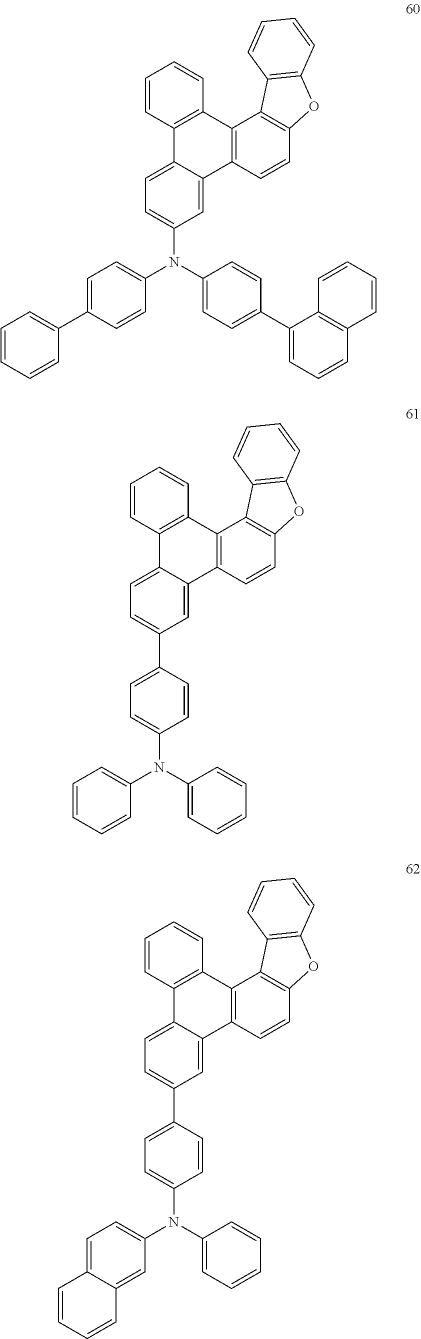 [SMILES: c1ccc(-c2ccc(N(c3ccc(-c4cccc5ccccc45)cc3)c3ccc4c5ccccc5c5c(ccc6oc7ccccc7c65)c4c3)cc2)cc1 ; c1ccc(N(c2ccccc2)c2ccc(-c3ccc4c5ccccc5c5c(ccc6oc7ccccc7c65)c4c3)cc2)cc1 ; c1ccc(N(c2ccc(-c3ccc4c5ccccc5c5c(ccc6oc7ccccc7c65)c4c3)cc2)c2ccc3ccccc3c2)cc1]

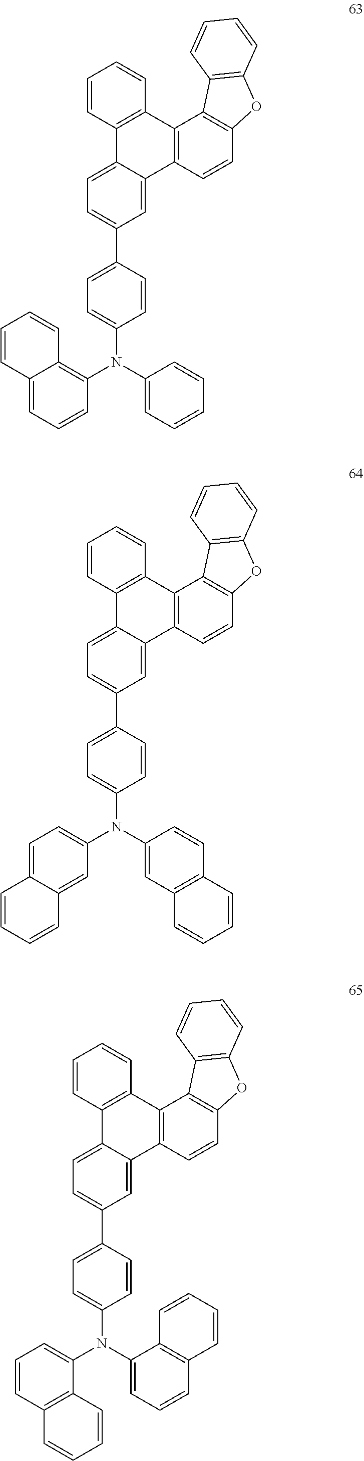 [SMILES: c1ccc(N(c2ccc(-c3ccc4c5ccccc5c5c(ccc6oc7ccccc7c65)c4c3)cc2)c2cccc3ccccc23)cc1 ; c1ccc2cc(N(c3ccc(-c4ccc5c6ccccc6c6c(ccc7oc8ccccc8c76)c5c4)cc3)c3ccc4ccccc4c3)ccc2c1 ; c1ccc2c(N(c3ccc(-c4ccc5c6ccccc6c6c(ccc7oc8ccccc8c76)c5c4)cc3)c3cccc4ccccc34)cccc2c1]

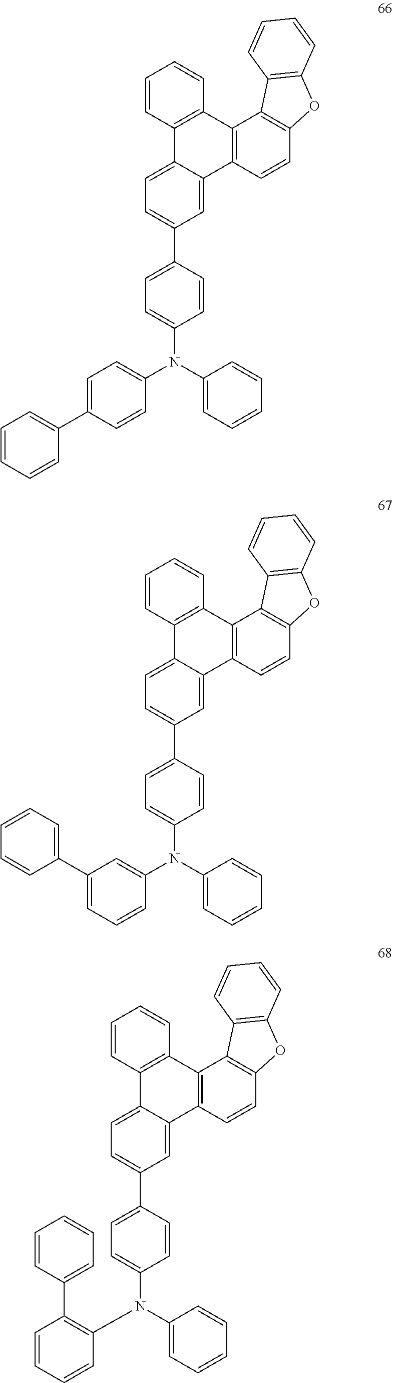

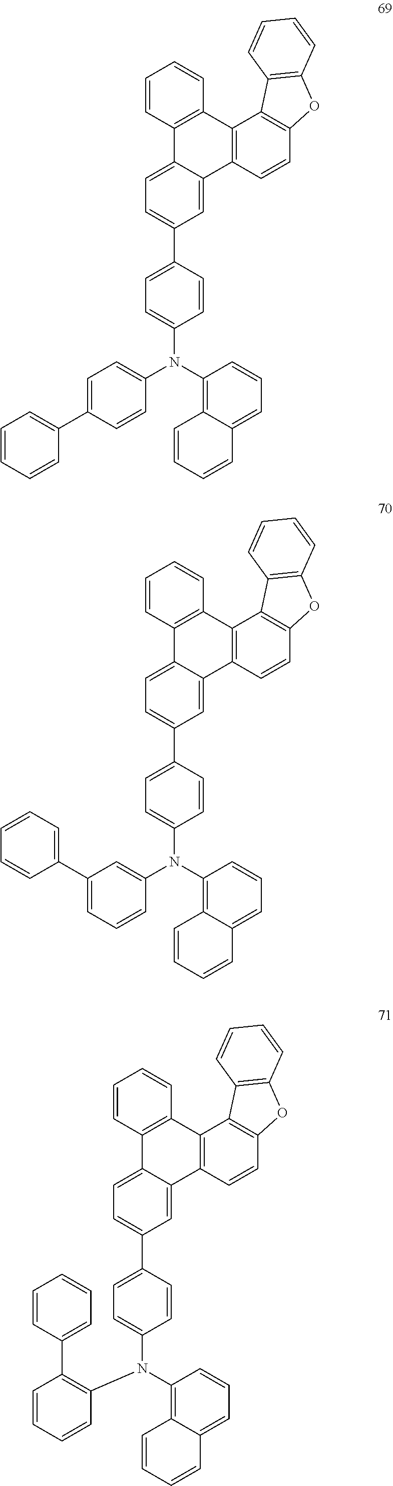 [SMILES: c1ccc(-c2ccc(N(c3ccc(-c4ccc5c6ccccc6c6c(ccc7oc8ccccc8c76)c5c4)cc3)c3cccc4ccccc34)cc2)cc1 ; c1ccc(-c2cccc(N(c3ccc(-c4ccc5c6ccccc6c6c(ccc7oc8ccccc8c76)c5c4)cc3)c3cccc4ccccc34)c2)cc1 ; c1ccc(-c2ccccc2N(c2ccc(-c3ccc4c5ccccc5c5c(ccc6oc7ccccc7c65)c4c3)cc2)c2cccc3ccccc23)cc1]

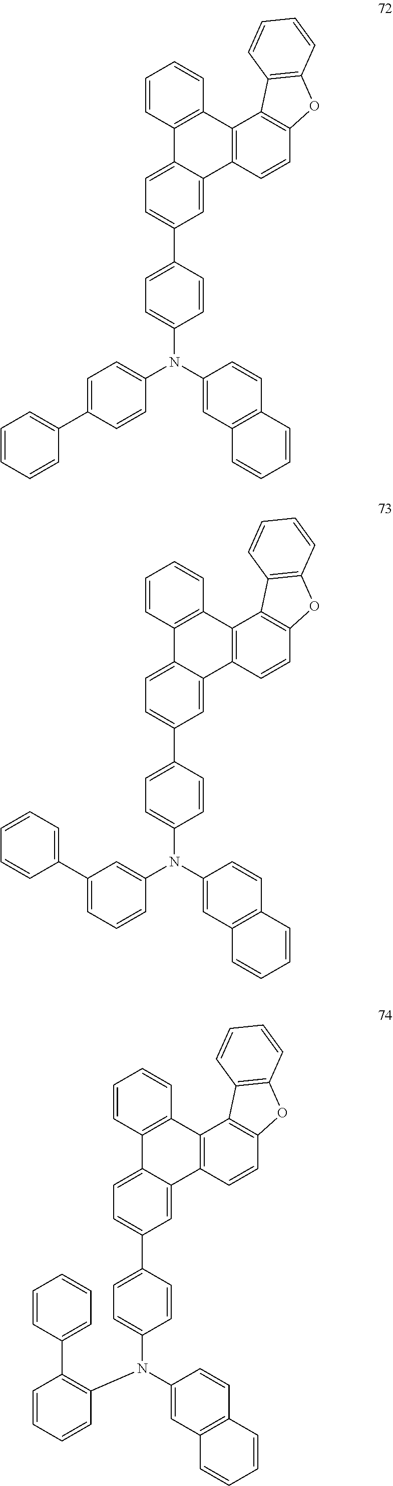

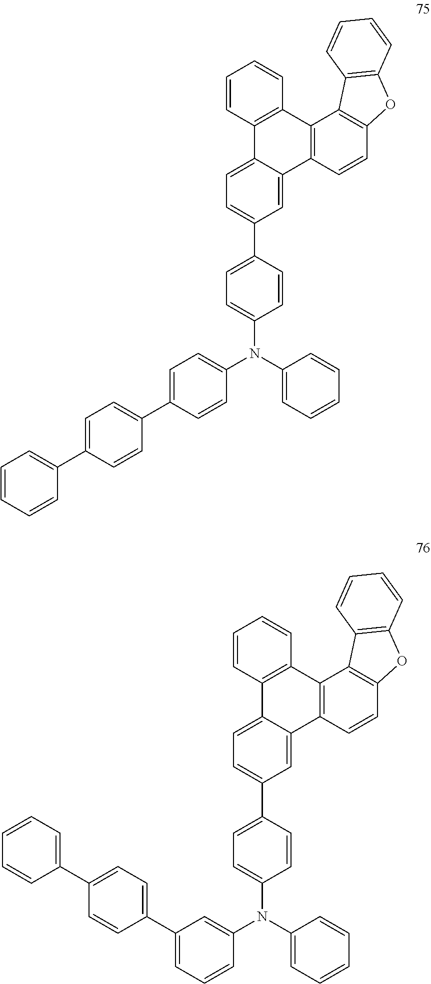 [SMILES: c1ccc(-c2ccc(-c3ccc(N(c4ccccc4)c4ccc(-c5ccc6c7ccccc7c7c(ccc8oc9ccccc9c87)c6c5)cc4)cc3)cc2)cc1 ; c1ccc(-c2ccc(-c3cccc(N(c4ccccc4)c4ccc(-c5ccc6c7ccccc7c7c(ccc8oc9ccccc9c87)c6c5)cc4)c3)cc2)cc1]

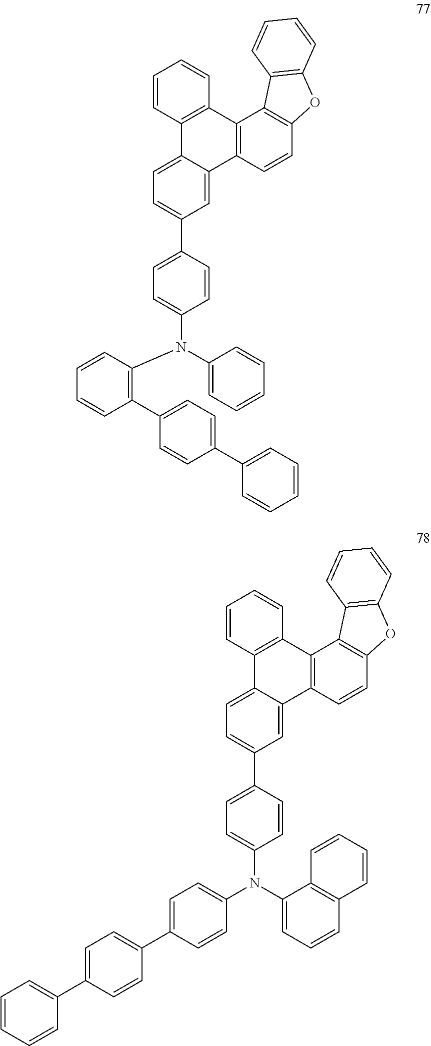 [SMILES: c1ccc(-c2ccc(-c3ccccc3N(c3ccccc3)c3ccc(-c4ccc5c6ccccc6c6c(ccc7oc8ccccc8c76)c5c4)cc3)cc2)cc1 ; c1ccc(-c2ccc(-c3ccc(N(c4ccc(-c5ccc6c7ccccc7c7c(ccc8oc9ccccc9c87)c6c5)cc4)c4cccc5ccccc45)cc3)cc2)cc1]

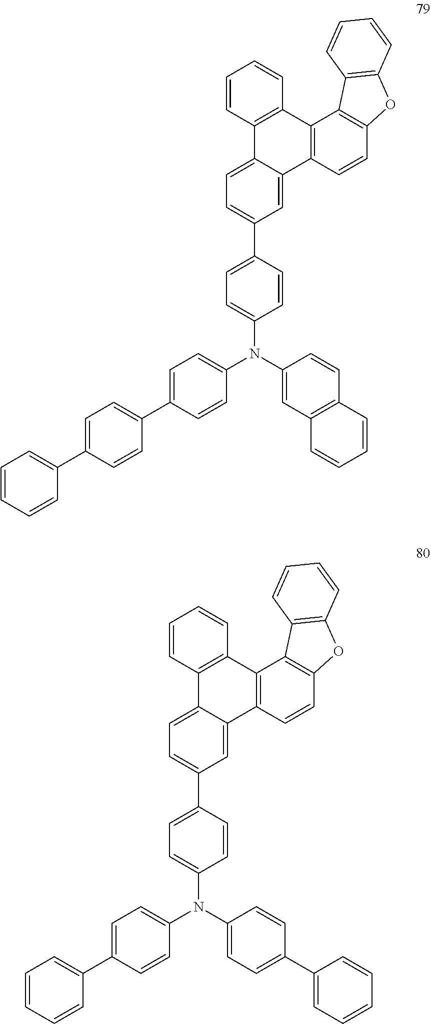 [SMILES: c1ccc(-c2ccc(-c3ccc(N(c4ccc(-c5ccc6c7ccccc7c7c(ccc8oc9ccccc9c87)c6c5)cc4)c4ccc5ccccc5c4)cc3)cc2)cc1 ; c1ccc(-c2ccc(N(c3ccc(-c4ccccc4)cc3)c3ccc(-c4ccc5c6ccccc6c6c(ccc7oc8ccccc8c76)c5c4)cc3)cc2)cc1]

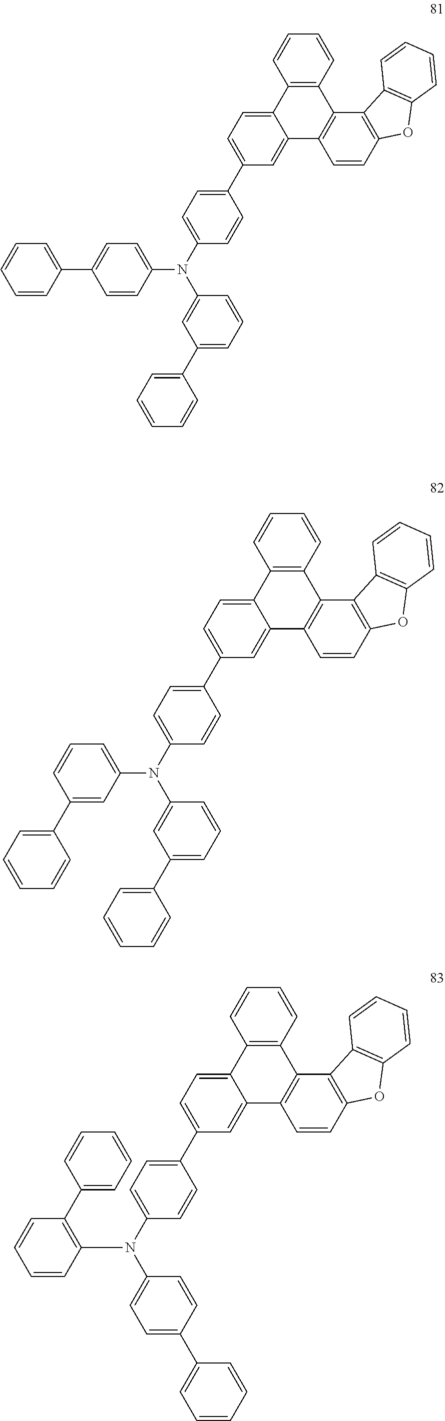 [SMILES: c1ccc(-c2ccc(N(c3ccc(-c4ccc5c6ccccc6c6c(ccc7oc8ccccc8c76)c5c4)cc3)c3cccc(-c4ccccc4)c3)cc2)cc1 ; c1ccc(-c2cccc(N(c3ccc(-c4ccc5c6ccccc6c6c(ccc7oc8ccccc8c76)c5c4)cc3)c3cccc(-c4ccccc4)c3)c2)cc1 ; c1ccc(-c2ccc(N(c3ccc(-c4ccc5c6ccccc6c6c(ccc7oc8ccccc8c76)c5c4)cc3)c3ccccc3-c3ccccc3)cc2)cc1]

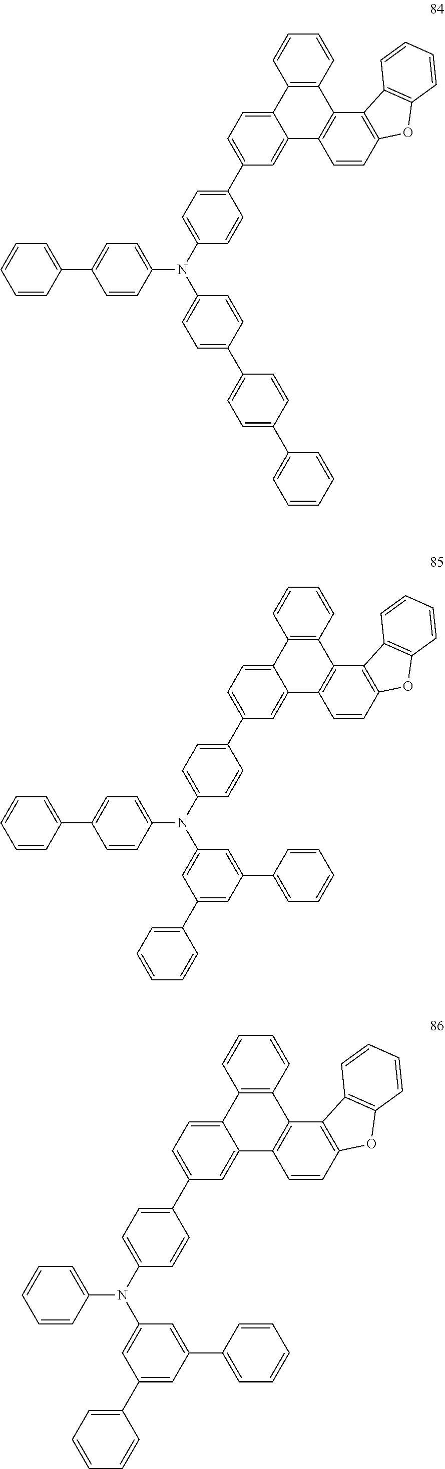 [SMILES: c1ccc(-c2ccc(-c3ccc(N(c4ccc(-c5ccccc5)cc4)c4ccc(-c5ccc6c7ccccc7c7c(ccc8oc9ccccc9c87)c6c5)cc4)cc3)cc2)cc1 ; c1ccc(-c2ccc(N(c3ccc(-c4ccc5c6ccccc6c6c(ccc7oc8ccccc8c76)c5c4)cc3)c3cc(-c4ccccc4)cc(-c4ccccc4)c3)cc2)cc1 ; c1ccc(-c2cc(-c3ccccc3)cc(N(c3ccccc3)c3ccc(-c4ccc5c6ccccc6c6c(ccc7oc8ccccc8c76)c5c4)cc3)c2)cc1]

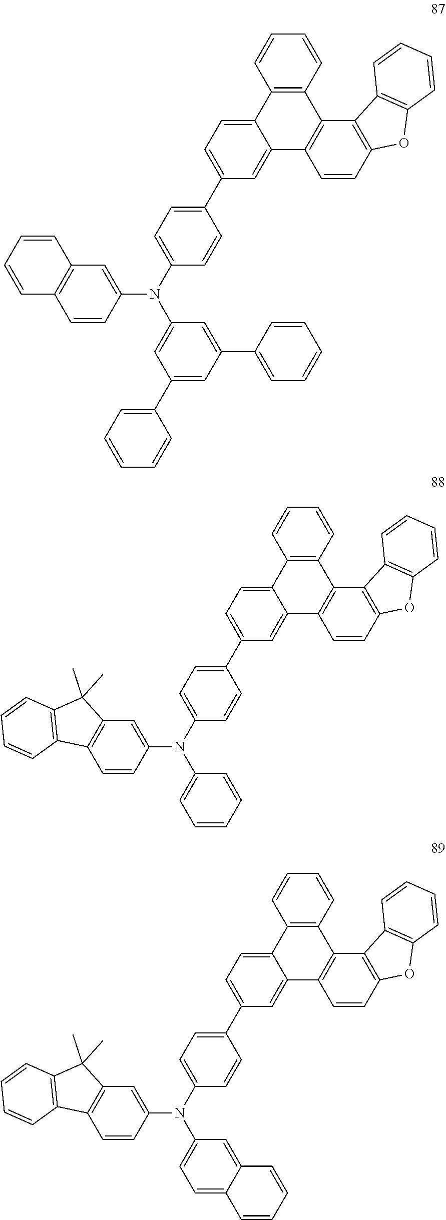 [SMILES: c1ccc(-c2cc(-c3ccccc3)cc(N(c3ccc(-c4ccc5c6ccccc6c6c(ccc7oc8ccccc8c76)c5c4)cc3)c3ccc4ccccc4c3)c2)cc1 ; CC1(C)c2ccccc2-c2ccc(N(c3ccccc3)c3ccc(-c4ccc5c6ccccc6c6c(ccc7oc8ccccc8c76)c5c4)cc3)cc21 ; CC1(C)c2ccccc2-c2ccc(N(c3ccc(-c4ccc5c6ccccc6c6c(ccc7oc8ccccc8c76)c5c4)cc3)c3ccc4ccccc4c3)cc21]

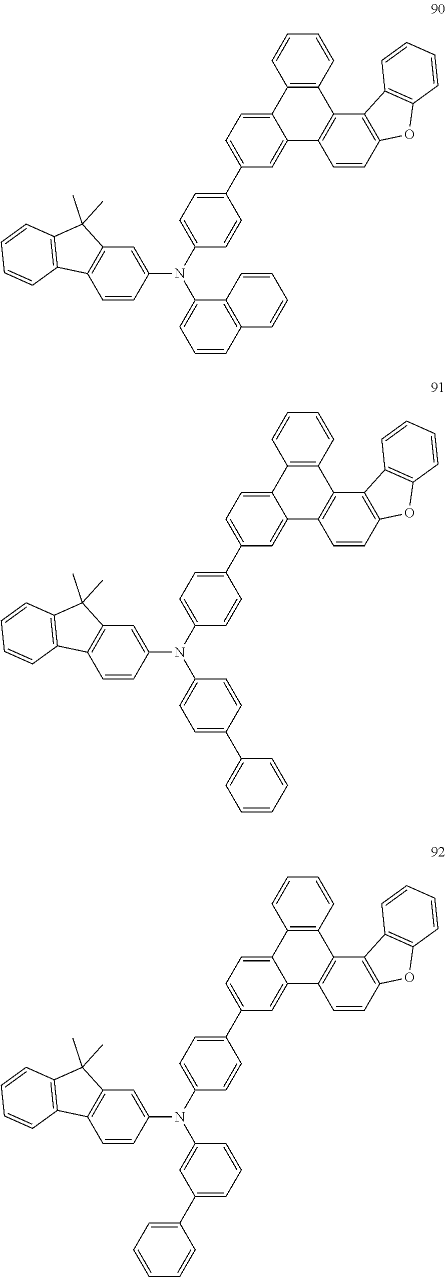 [SMILES: CC1(C)c2ccccc2-c2ccc(N(c3ccc(-c4ccc5c6ccccc6c6c(ccc7oc8ccccc8c76)c5c4)cc3)c3cccc4ccccc34)cc21 ; CC1(C)c2ccccc2-c2ccc(N(c3ccc(-c4ccccc4)cc3)c3ccc(-c4ccc5c6ccccc6c6c(ccc7oc8ccccc8c76)c5c4)cc3)cc21 ; CC1(C)c2ccccc2-c2ccc(N(c3ccc(-c4ccc5c6ccccc6c6c(ccc7oc8ccccc8c76)c5c4)cc3)c3cccc(-c4ccccc4)c3)cc21]

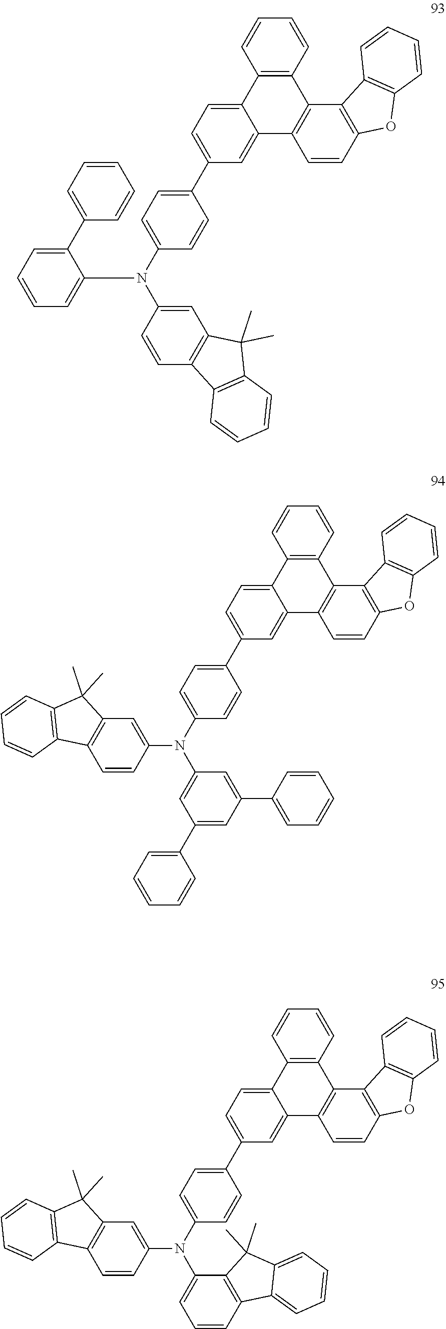 [SMILES: CC1(C)c2ccccc2-c2ccc(N(c3ccc(-c4ccc5c6ccccc6c6c(ccc7oc8ccccc8c76)c5c4)cc3)c3ccccc3-c3ccccc3)cc21 ; CC1(C)c2ccccc2-c2ccc(N(c3ccc(-c4ccc5c6ccccc6c6c(ccc7oc8ccccc8c76)c5c4)cc3)c3cc(-c4ccccc4)cc(-c4ccccc4)c3)cc21 ; CC1(C)c2ccccc2-c2ccc(N(c3ccc(-c4ccc5c6ccccc6c6c(ccc7oc8ccccc8c76)c5c4)cc3)c3cccc4c3C(C)(C)c3ccccc3-4)cc21]

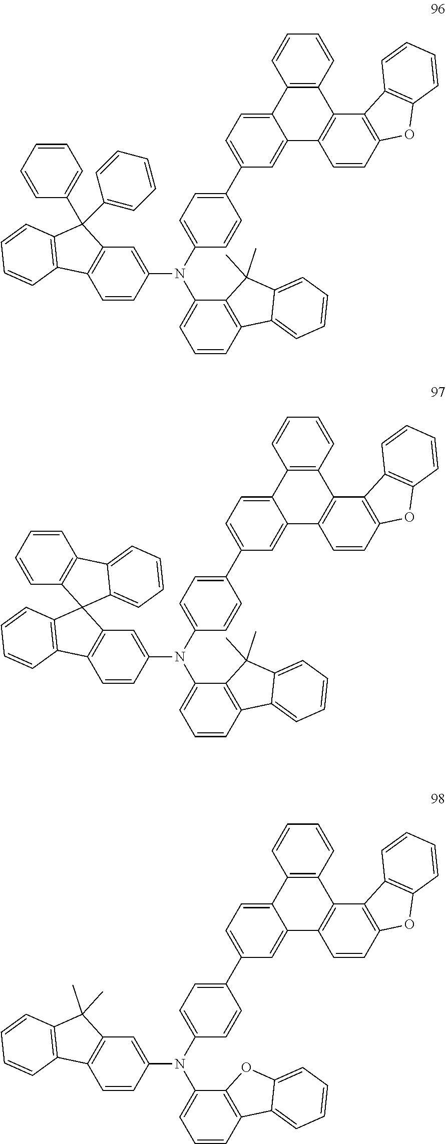 [SMILES: CC1(C)c2ccccc2-c2cccc(N(c3ccc(-c4ccc5c6ccccc6c6c(ccc7oc8ccccc8c76)c5c4)cc3)c3ccc4c(c3)C(c3ccccc3)(c3ccccc3)c3ccccc3-4)c21 ; CC1(C)c2ccccc2-c2cccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)c21 ; CC1(C)c2ccccc2-c2ccc(N(c3ccc(-c4ccc5c6ccccc6c6c(ccc7oc8ccccc8c76)c5c4)cc3)c3cccc4c3oc3ccccc34)cc21]

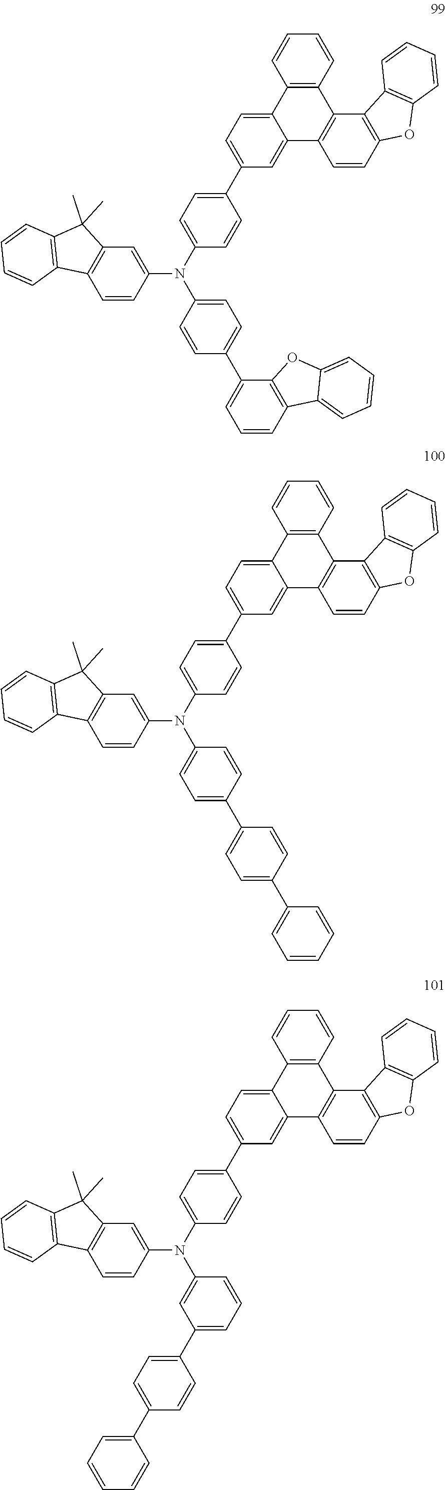 [SMILES: CC1(C)c2ccccc2-c2ccc(N(c3ccc(-c4ccc5c6ccccc6c6c(ccc7oc8ccccc8c76)c5c4)cc3)c3ccc(-c4cccc5c4oc4ccccc45)cc3)cc21 ; CC1(C)c2ccccc2-c2ccc(N(c3ccc(-c4ccc(-c5ccccc5)cc4)cc3)c3ccc(-c4ccc5c6ccccc6c6c(ccc7oc8ccccc8c76)c5c4)cc3)cc21 ; CC1(C)c2ccccc2-c2ccc(N(c3ccc(-c4ccc5c6ccccc6c6c(ccc7oc8ccccc8c76)c5c4)cc3)c3cccc(-c4ccc(-c5ccccc5)cc4)c3)cc21]

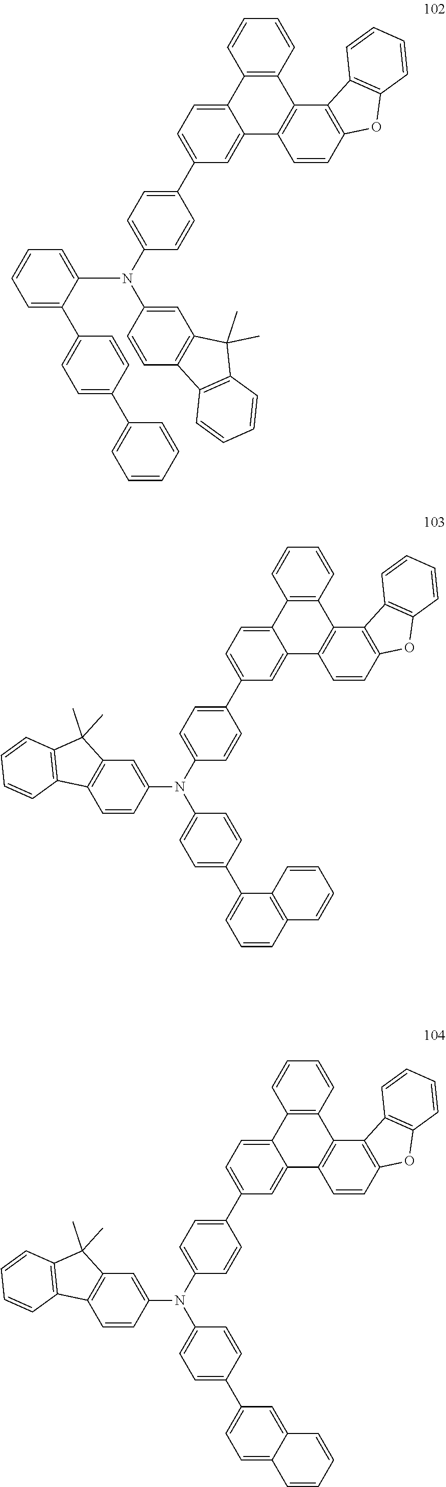 [SMILES: CC1(C)c2ccccc2-c2ccc(N(c3ccc(-c4ccc5c6ccccc6c6c(ccc7oc8ccccc8c76)c5c4)cc3)c3ccccc3-c3ccc(-c4ccccc4)cc3)cc21 ; CC1(C)c2ccccc2-c2ccc(N(c3ccc(-c4ccc5c6ccccc6c6c(ccc7oc8ccccc8c76)c5c4)cc3)c3ccc(-c4cccc5ccccc45)cc3)cc21 ; CC1(C)c2ccccc2-c2ccc(N(c3ccc(-c4ccc5ccccc5c4)cc3)c3ccc(-c4ccc5c6ccccc6c6c(ccc7oc8ccccc8c76)c5c4)cc3)cc21]

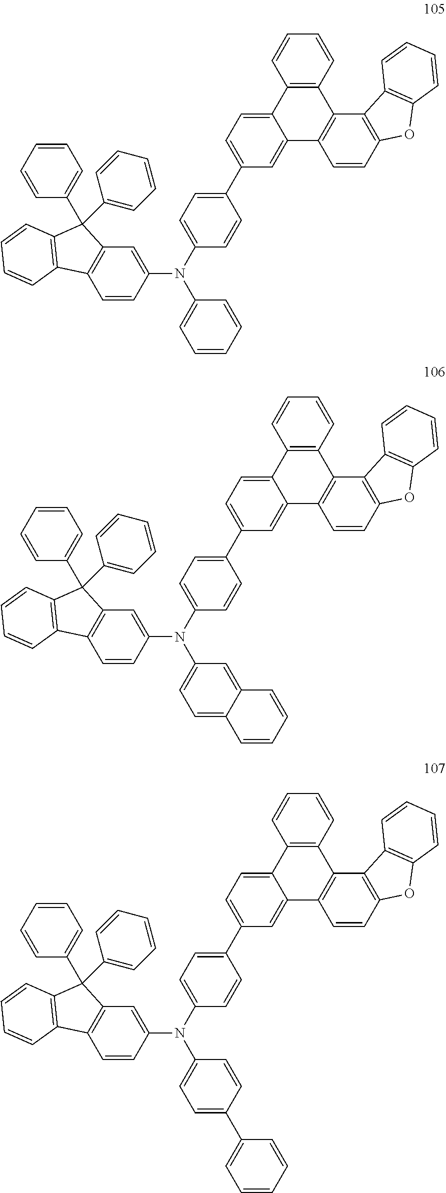 [SMILES: c1ccc(N(c2ccc(-c3ccc4c5ccccc5c5c(ccc6oc7ccccc7c65)c4c3)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccc6c7ccccc7c7c(ccc8oc9ccccc9c87)c6c5)cc4)c4ccc5ccccc5c4)cc32)cc1 ; c1ccc(-c2ccc(N(c3ccc(-c4ccc5c6ccccc6c6c(ccc7oc8ccccc8c76)c5c4)cc3)c3ccc4c(c3)C(c3ccccc3)(c3ccccc3)c3ccccc3-4)cc2)cc1]

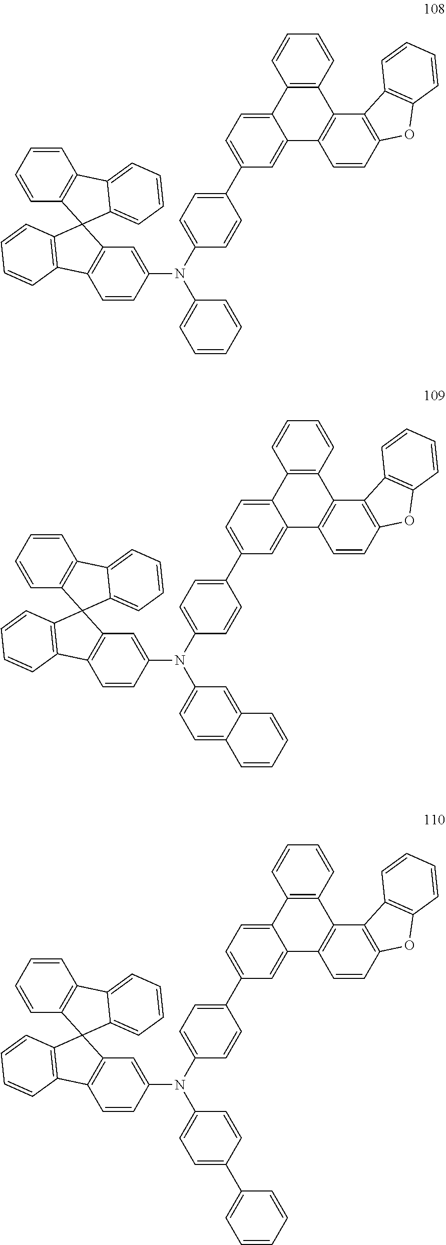 [SMILES: c1ccc(N(c2ccc(-c3ccc4c5ccccc5c5c(ccc6oc7ccccc7c65)c4c3)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4ccc5c6ccccc6c6c(ccc7oc8ccccc8c76)c5c4)cc3)c3ccc4ccccc4c3)cc21 ; c1ccc(-c2ccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)cc2)cc1]

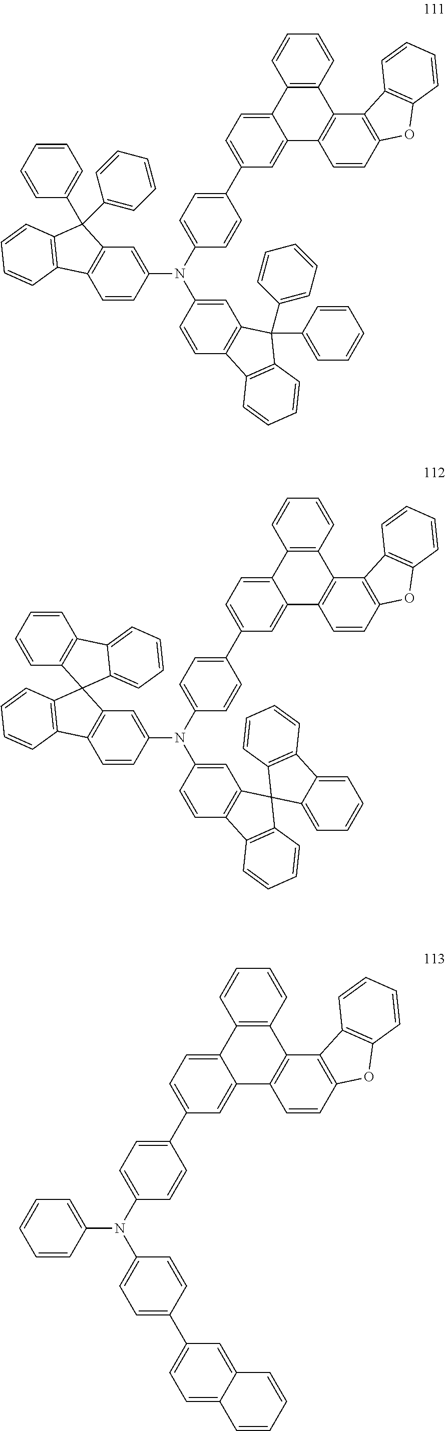 [SMILES: c1ccc(C2(c3ccccc3)c3ccccc3-c3ccc(N(c4ccc(-c5ccc6c7ccccc7c7c(ccc8oc9ccccc9c87)c6c5)cc4)c4ccc5c(c4)C(c4ccccc4)(c4ccccc4)c4ccccc4-5)cc32)cc1 ; c1ccc2c(c1)-c1ccccc1C21c2ccccc2-c2ccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)cc21 ; c1ccc(N(c2ccc(-c3ccc4ccccc4c3)cc2)c2ccc(-c3ccc4c5ccccc5c5c(ccc6oc7ccccc7c65)c4c3)cc2)cc1]

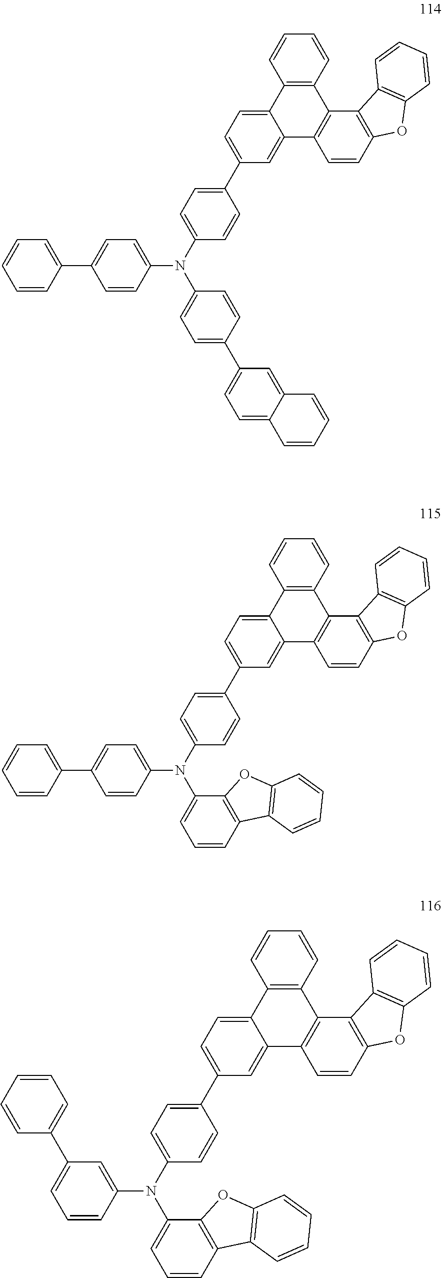 [SMILES: c1ccc(-c2ccc(N(c3ccc(-c4ccc5ccccc5c4)cc3)c3ccc(-c4ccc5c6ccccc6c6c(ccc7oc8ccccc8c76)c5c4)cc3)cc2)cc1 ; c1ccc(-c2ccc(N(c3ccc(-c4ccc5c6ccccc6c6c(ccc7oc8ccccc8c76)c5c4)cc3)c3cccc4c3oc3ccccc34)cc2)cc1 ; c1ccc(-c2cccc(N(c3ccc(-c4ccc5c6ccccc6c6c(ccc7oc8ccccc8c76)c5c4)cc3)c3cccc4c3oc3ccccc34)c2)cc1]

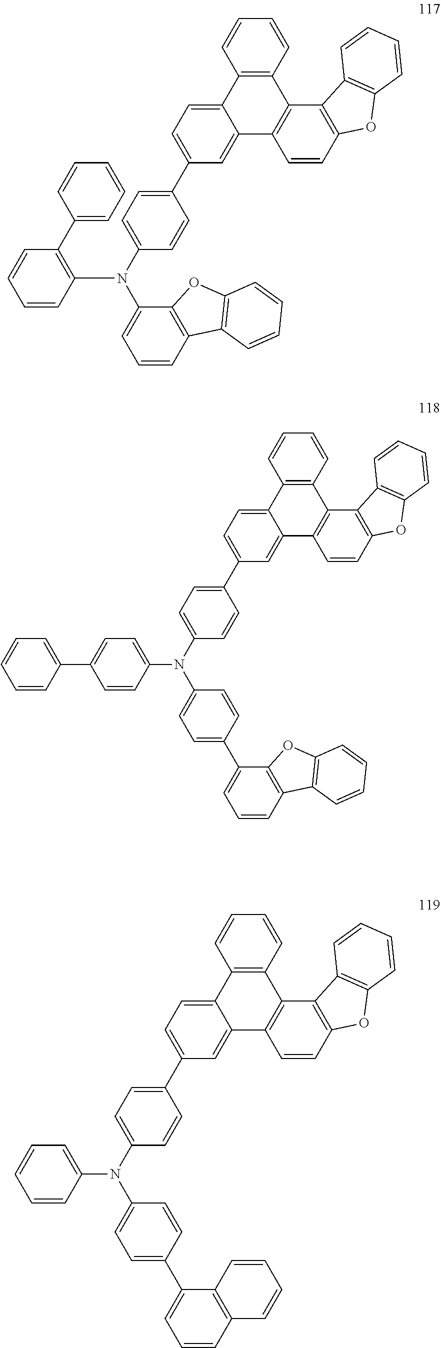 [SMILES: c1ccc(-c2ccccc2N(c2ccc(-c3ccc4c5ccccc5c5c(ccc6oc7ccccc7c65)c4c3)cc2)c2cccc3c2oc2ccccc23)cc1 ; c1ccc(-c2ccc(N(c3ccc(-c4ccc5c6ccccc6c6c(ccc7oc8ccccc8c76)c5c4)cc3)c3ccc(-c4cccc5c4oc4ccccc45)cc3)cc2)cc1 ; c1ccc(N(c2ccc(-c3ccc4c5ccccc5c5c(ccc6oc7ccccc7c65)c4c3)cc2)c2ccc(-c3cccc4ccccc34)cc2)cc1]

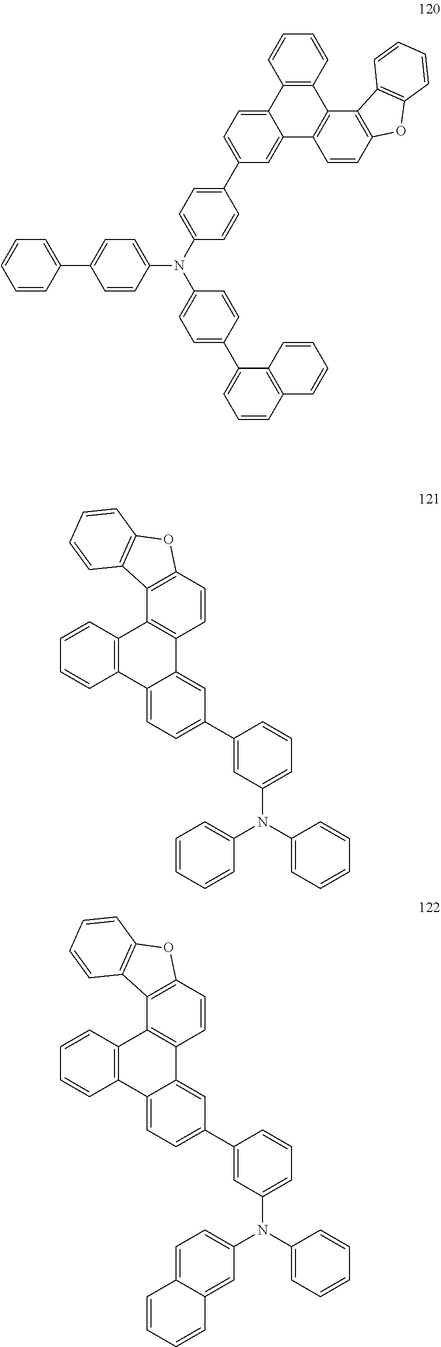 [SMILES: c1ccc(-c2ccc(N(c3ccc(-c4ccc5c6ccccc6c6c(ccc7oc8ccccc8c76)c5c4)cc3)c3ccc(-c4cccc5ccccc45)cc3)cc2)cc1 ; c1ccc(N(c2ccccc2)c2cccc(-c3ccc4c5ccccc5c5c(ccc6oc7ccccc7c65)c4c3)c2)cc1 ; c1ccc(N(c2cccc(-c3ccc4c5ccccc5c5c(ccc6oc7ccccc7c65)c4c3)c2)c2ccc3ccccc3c2)cc1]

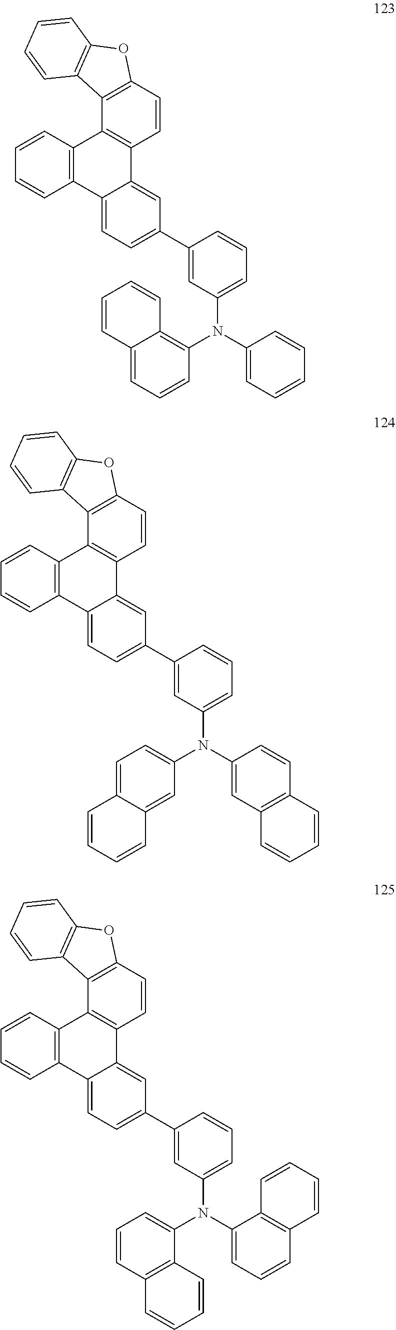 [SMILES: c1ccc(N(c2cccc(-c3ccc4c5ccccc5c5c(ccc6oc7ccccc7c65)c4c3)c2)c2cccc3ccccc23)cc1 ; c1cc(-c2ccc3c4ccccc4c4c(ccc5oc6ccccc6c54)c3c2)cc(N(c2ccc3ccccc3c2)c2ccc3ccccc3c2)c1 ; c1cc(-c2ccc3c4ccccc4c4c(ccc5oc6ccccc6c54)c3c2)cc(N(c2cccc3ccccc23)c2cccc3ccccc23)c1]

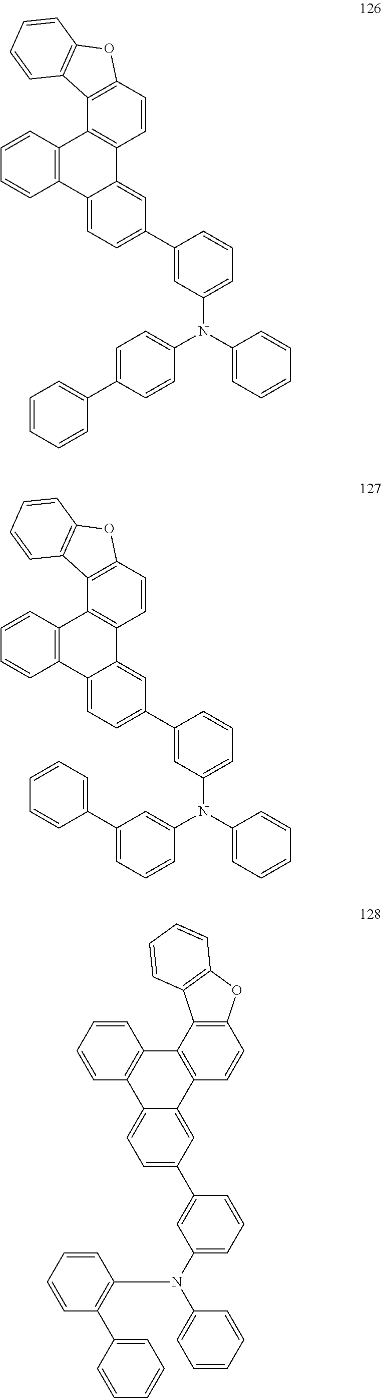 [SMILES: c1ccc(-c2ccc(N(c3ccccc3)c3cccc(-c4ccc5c6ccccc6c6c(ccc7oc8ccccc8c76)c5c4)c3)cc2)cc1 ; c1ccc(-c2cccc(N(c3ccccc3)c3cccc(-c4ccc5c6ccccc6c6c(ccc7oc8ccccc8c76)c5c4)c3)c2)cc1 ; c1ccc(-c2ccccc2N(c2ccccc2)c2cccc(-c3ccc4c5ccccc5c5c(ccc6oc7ccccc7c65)c4c3)c2)cc1]

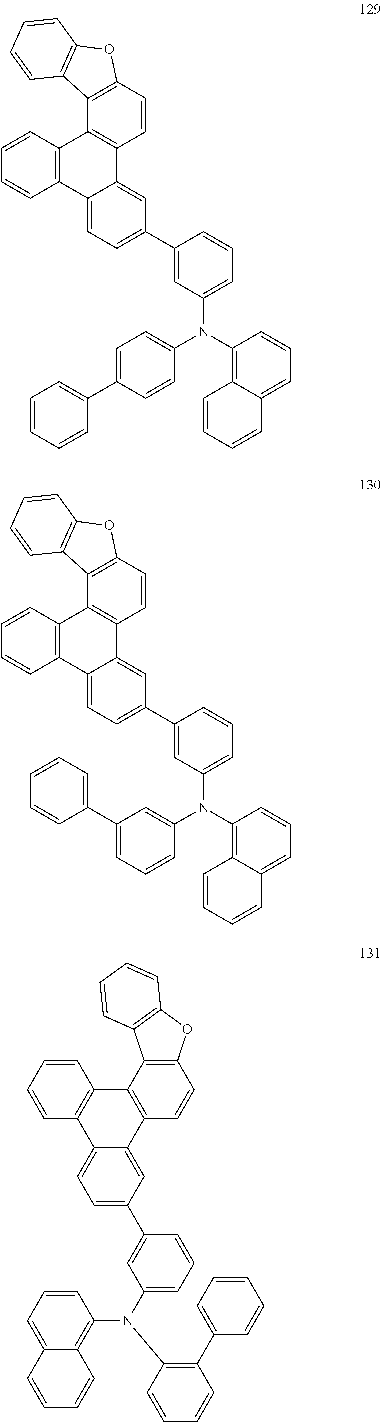

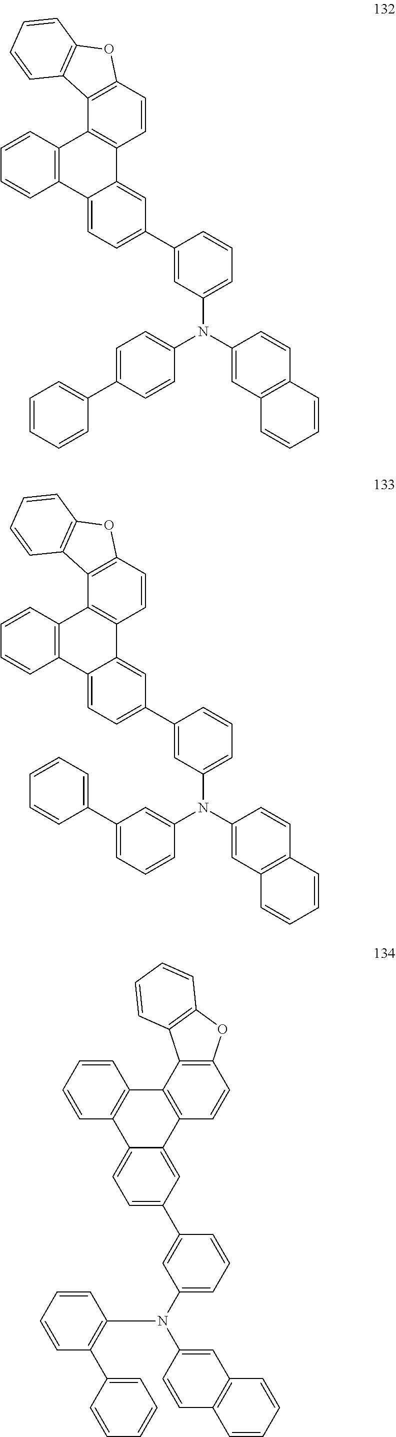

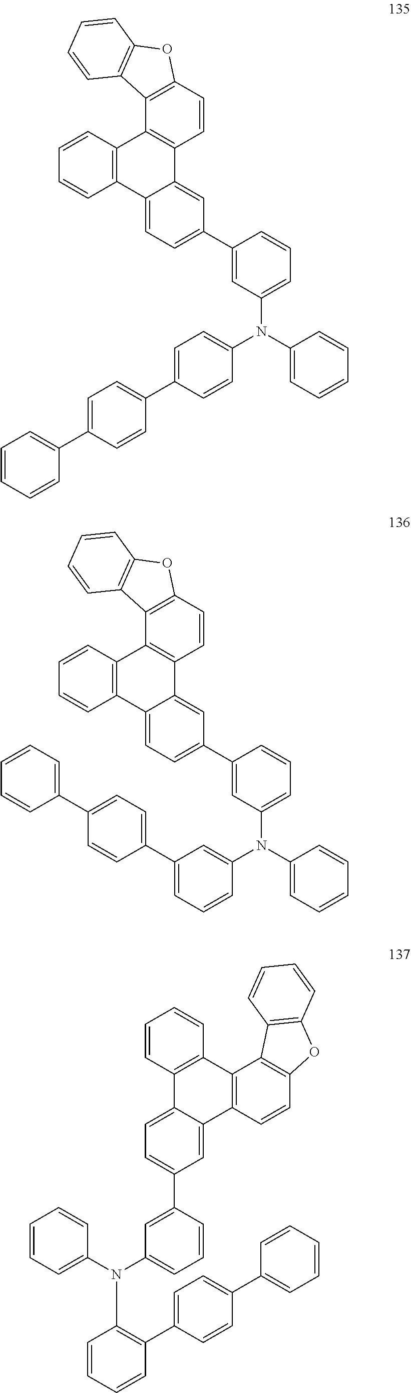 [SMILES: c1ccc(-c2ccc(-c3ccc(N(c4ccccc4)c4cccc(-c5ccc6c7ccccc7c7c(ccc8oc9ccccc9c87)c6c5)c4)cc3)cc2)cc1 ; c1ccc(-c2ccc(-c3cccc(N(c4ccccc4)c4cccc(-c5ccc6c7ccccc7c7c(ccc8oc9ccccc9c87)c6c5)c4)c3)cc2)cc1 ; c1ccc(-c2ccc(-c3ccccc3N(c3ccccc3)c3cccc(-c4ccc5c6ccccc6c6c(ccc7oc8ccccc8c76)c5c4)c3)cc2)cc1]

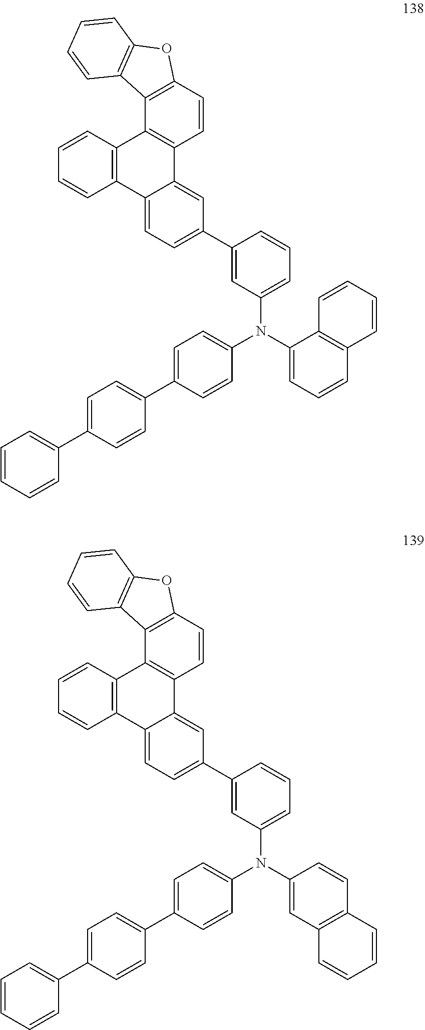 [SMILES: c1ccc(-c2ccc(-c3ccc(N(c4cccc(-c5ccc6c7ccccc7c7c(ccc8oc9ccccc9c87)c6c5)c4)c4cccc5ccccc45)cc3)cc2)cc1 ; c1ccc(-c2ccc(-c3ccc(N(c4cccc(-c5ccc6c7ccccc7c7c(ccc8oc9ccccc9c87)c6c5)c4)c4ccc5ccccc5c4)cc3)cc2)cc1]

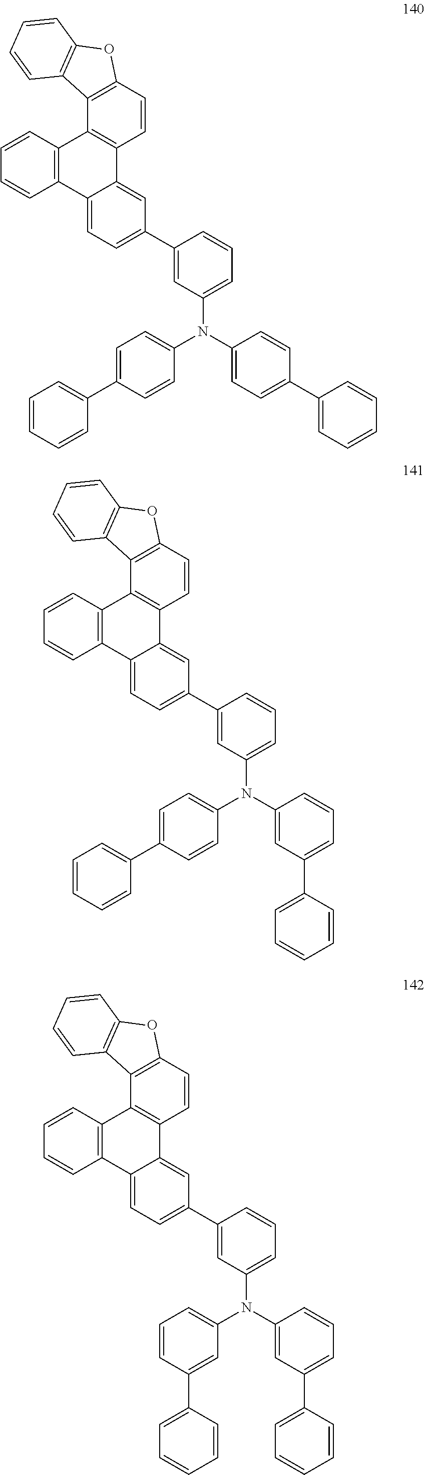

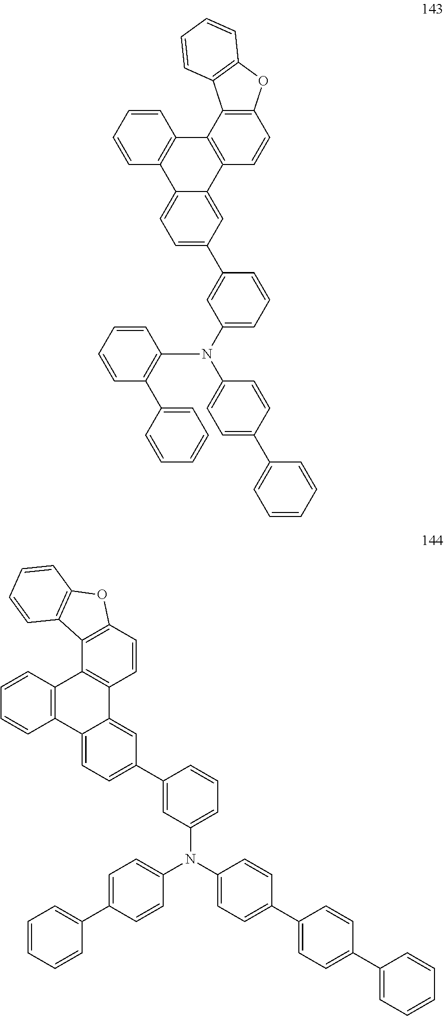 [SMILES: c1ccc(-c2ccc(N(c3cccc(-c4ccc5c6ccccc6c6c(ccc7oc8ccccc8c76)c5c4)c3)c3ccccc3-c3ccccc3)cc2)cc1 ; c1ccc(-c2ccc(-c3ccc(N(c4ccc(-c5ccccc5)cc4)c4cccc(-c5ccc6c7ccccc7c7c(ccc8oc9ccccc9c87)c6c5)c4)cc3)cc2)cc1]

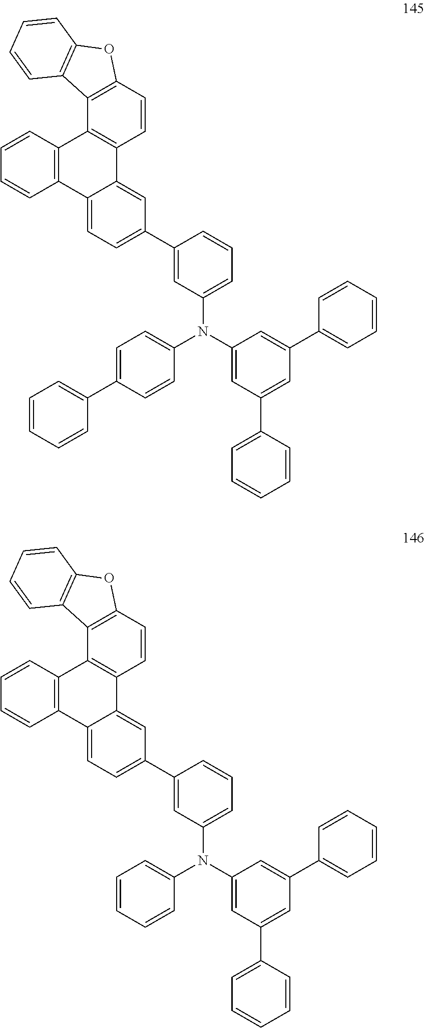 [SMILES: c1ccc(-c2ccc(N(c3cccc(-c4ccc5c6ccccc6c6c(ccc7oc8ccccc8c76)c5c4)c3)c3cc(-c4ccccc4)cc(-c4ccccc4)c3)cc2)cc1 ; c1ccc(-c2cc(-c3ccccc3)cc(N(c3ccccc3)c3cccc(-c4ccc5c6ccccc6c6c(ccc7oc8ccccc8c76)c5c4)c3)c2)cc1]

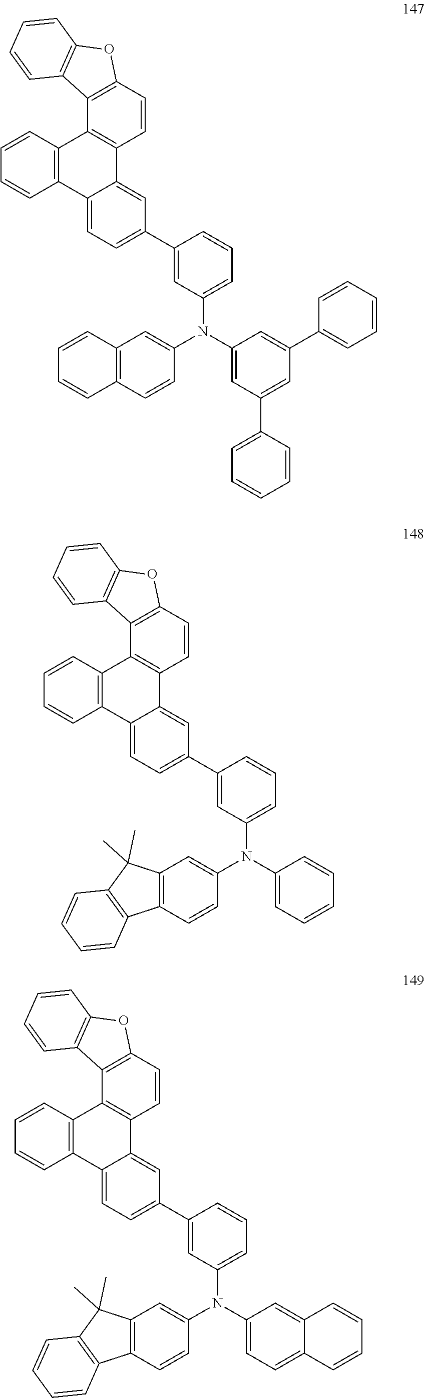

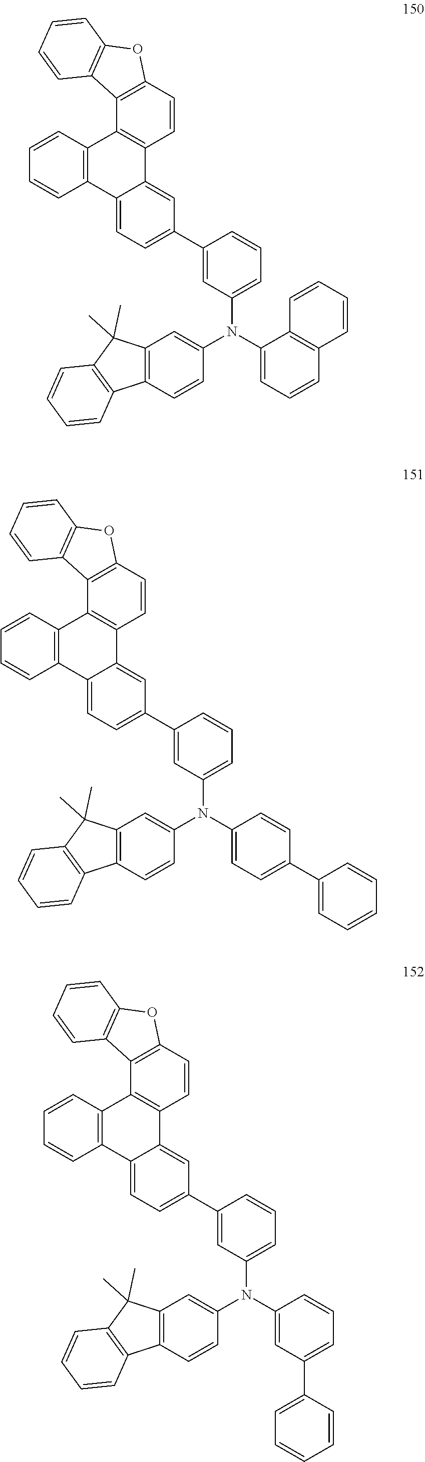 [SMILES: CC1(C)c2ccccc2-c2ccc(N(c3cccc(-c4ccc5c6ccccc6c6c(ccc7oc8ccccc8c76)c5c4)c3)c3cccc4ccccc34)cc21 ; CC1(C)c2ccccc2-c2ccc(N(c3ccc(-c4ccccc4)cc3)c3cccc(-c4ccc5c6ccccc6c6c(ccc7oc8ccccc8c76)c5c4)c3)cc21 ; CC1(C)c2ccccc2-c2ccc(N(c3cccc(-c4ccccc4)c3)c3cccc(-c4ccc5c6ccccc6c6c(ccc7oc8ccccc8c76)c5c4)c3)cc21]

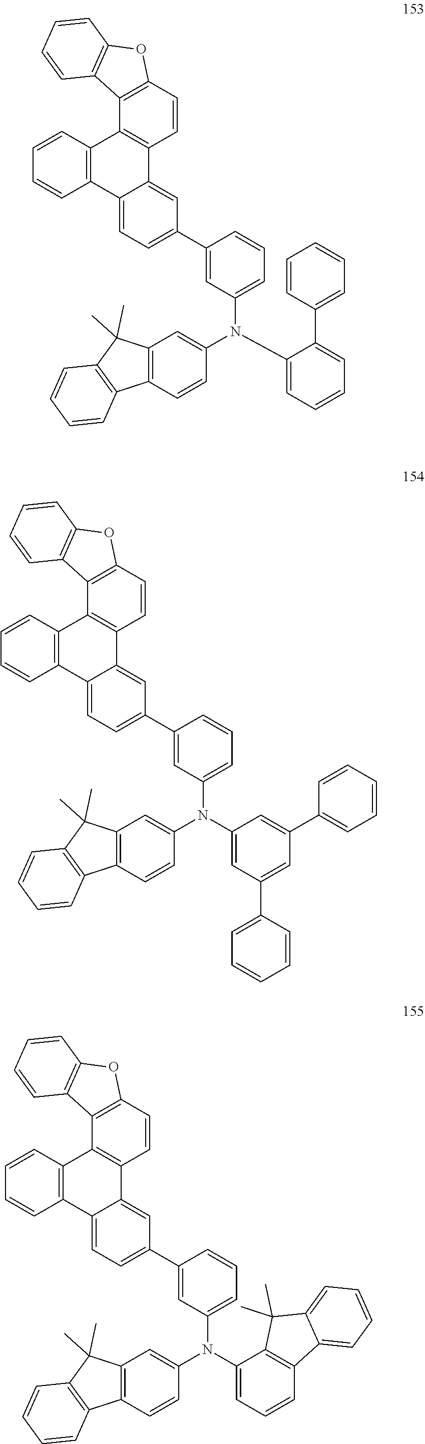 [SMILES: CC1(C)c2ccccc2-c2ccc(N(c3cccc(-c4ccc5c6ccccc6c6c(ccc7oc8ccccc8c76)c5c4)c3)c3ccccc3-c3ccccc3)cc21 ; CC1(C)c2ccccc2-c2ccc(N(c3cccc(-c4ccc5c6ccccc6c6c(ccc7oc8ccccc8c76)c5c4)c3)c3cc(-c4ccccc4)cc(-c4ccccc4)c3)cc21 ; CC1(C)c2ccccc2-c2ccc(N(c3cccc(-c4ccc5c6ccccc6c6c(ccc7oc8ccccc8c76)c5c4)c3)c3cccc4c3C(C)(C)c3ccccc3-4)cc21]

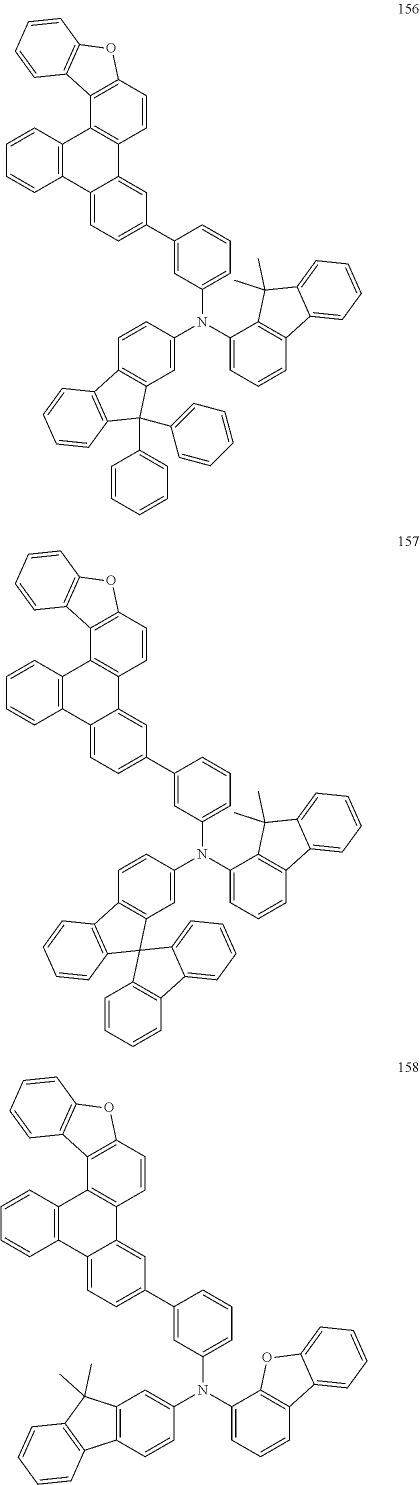 [SMILES: CC1(C)c2ccccc2-c2cccc(N(c3cccc(-c4ccc5c6ccccc6c6c(ccc7oc8ccccc8c76)c5c4)c3)c3ccc4c(c3)C(c3ccccc3)(c3ccccc3)c3ccccc3-4)c21 ; CC1(C)c2ccccc2-c2cccc(N(c3cccc(-c4ccc5c6ccccc6c6c(ccc7oc8ccccc8c76)c5c4)c3)c3ccc4c(c3)C3(c5ccccc5-c5ccccc53)c3ccccc3-4)c21 ; CC1(C)c2ccccc2-c2ccc(N(c3cccc(-c4ccc5c6ccccc6c6c(ccc7oc8ccccc8c76)c5c4)c3)c3cccc4c3oc3ccccc34)cc21]

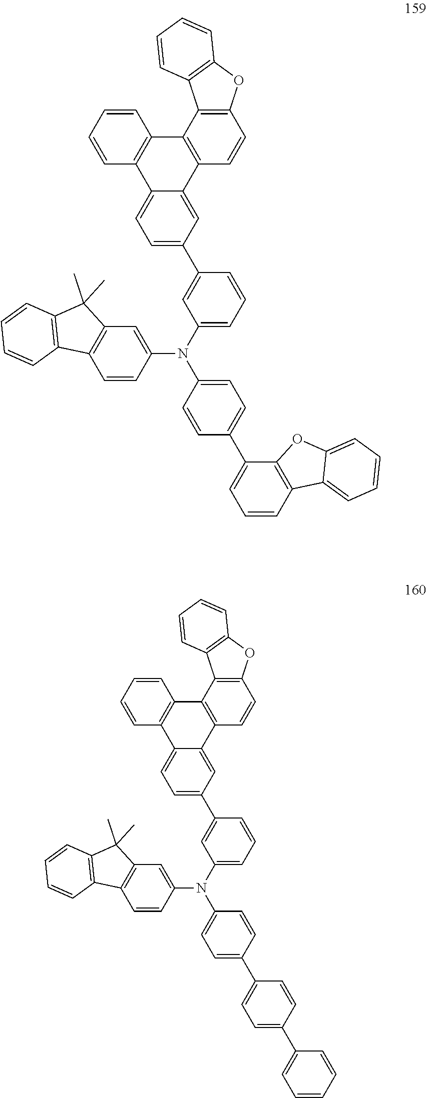 [SMILES: CC1(C)c2ccccc2-c2ccc(N(c3ccc(-c4cccc5c4oc4ccccc45)cc3)c3cccc(-c4ccc5c6ccccc6c6c(ccc7oc8ccccc8c76)c5c4)c3)cc21 ; CC1(C)c2ccccc2-c2ccc(N(c3ccc(-c4ccc(-c5ccccc5)cc4)cc3)c3cccc(-c4ccc5c6ccccc6c6c(ccc7oc8ccccc8c76)c5c4)c3)cc21]

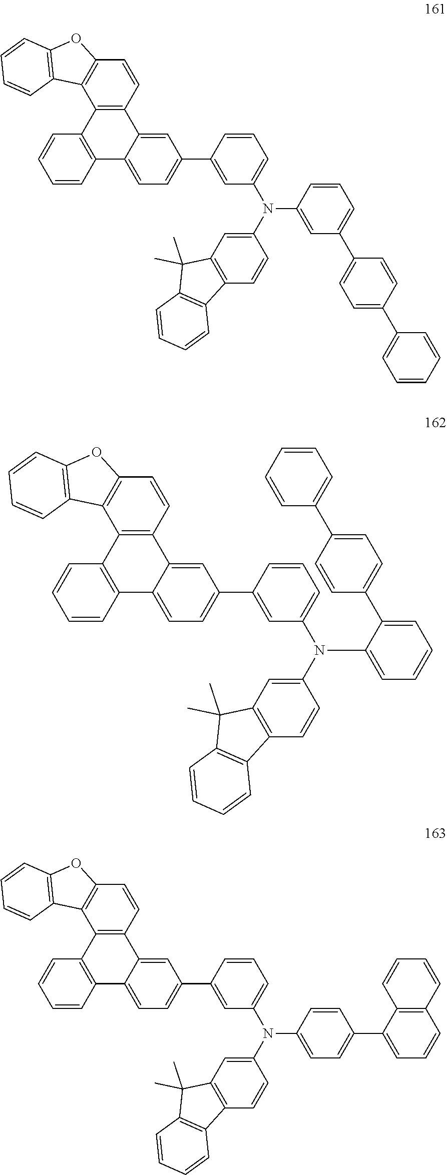 [SMILES: CC1(C)c2ccccc2-c2ccc(N(c3cccc(-c4ccc(-c5ccccc5)cc4)c3)c3cccc(-c4ccc5c6ccccc6c6c(ccc7oc8ccccc8c76)c5c4)c3)cc21 ; CC1(C)c2ccccc2-c2ccc(N(c3cccc(-c4ccc5c6ccccc6c6c(ccc7oc8ccccc8c76)c5c4)c3)c3ccccc3-c3ccc(-c4ccccc4)cc3)cc21 ; CC1(C)c2ccccc2-c2ccc(N(c3ccc(-c4cccc5ccccc45)cc3)c3cccc(-c4ccc5c6ccccc6c6c(ccc7oc8ccccc8c76)c5c4)c3)cc21]

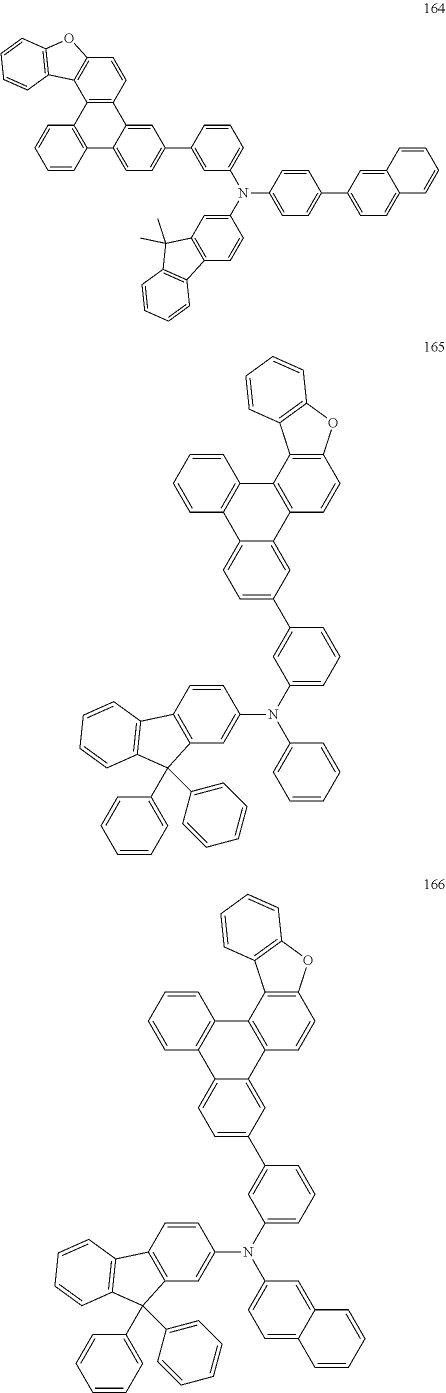 [SMILES: CC1(C)c2ccccc2-c2ccc(N(c3ccc(-c4ccc5ccccc5c4)cc3)c3cccc(-c4ccc5c6ccccc6c6c(ccc7oc8ccccc8c76)c5c4)c3)cc21 ; c1ccc(N(c2cccc(-c3ccc4c5ccccc5c5c(ccc6oc7ccccc7c65)c4c3)c2)c2ccc3c(c2)C(c2ccccc2)(c2ccccc2)c2ccccc2-3)cc1 ; c1ccc(C2(c3ccccc3)c3ccccc3-c3ccc(N(c4cccc(-c5ccc6c7ccccc7c7c(ccc8oc9ccccc9c87)c6c5)c4)c4ccc5ccccc5c4)cc32)cc1]

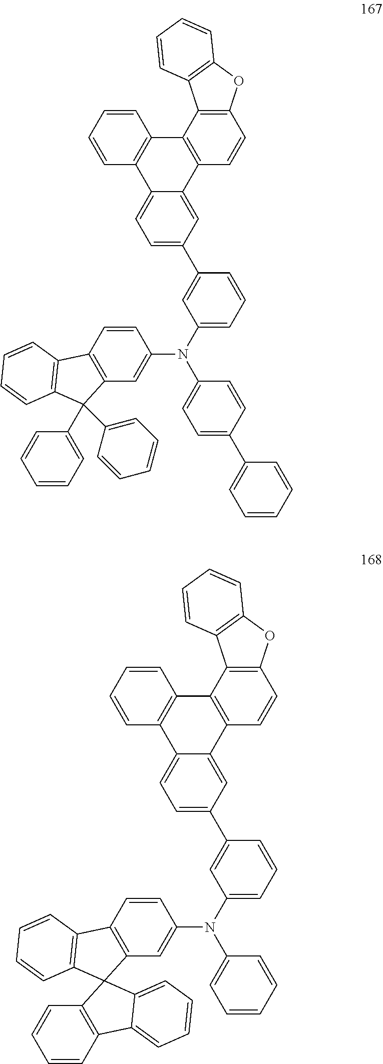 [SMILES: c1ccc(-c2ccc(N(c3cccc(-c4ccc5c6ccccc6c6c(ccc7oc8ccccc8c76)c5c4)c3)c3ccc4c(c3)C(c3ccccc3)(c3ccccc3)c3ccccc3-4)cc2)cc1 ; c1ccc(N(c2cccc(-c3ccc4c5ccccc5c5c(ccc6oc7ccccc7c65)c4c3)c2)c2ccc3c(c2)C2(c4ccccc4-c4ccccc42)c2ccccc2-3)cc1]

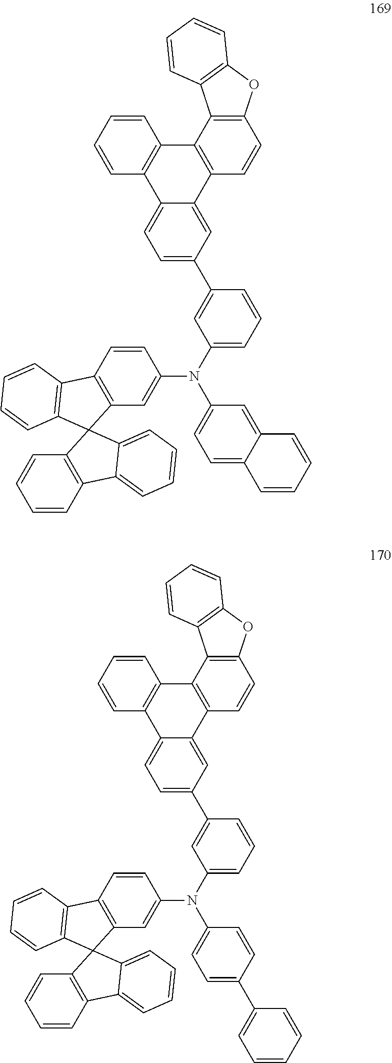 [SMILES: c1cc(-c2ccc3c4ccccc4c4c(ccc5oc6ccccc6c54)c3c2)cc(N(c2ccc3c(c2)C2(c4ccccc4-c4ccccc42)c2ccccc2-3)c2ccc3ccccc3c2)c1 ; c1ccc(-c2ccc(N(c3cccc(-c4ccc5c6ccccc6c6c(ccc7oc8ccccc8c76)c5c4)c3)c3ccc4c(c3)C3(c5ccccc5-c5ccccc53)c3ccccc3-4)cc2)cc1]

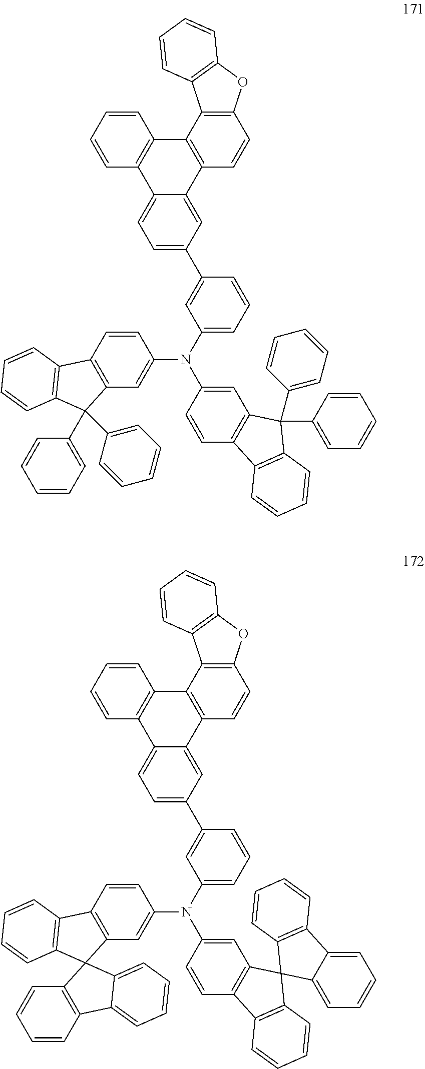 [SMILES: c1ccc(C2(c3ccccc3)c3ccccc3-c3ccc(N(c4cccc(-c5ccc6c7ccccc7c7c(ccc8oc9ccccc9c87)c6c5)c4)c4ccc5c(c4)C(c4ccccc4)(c4ccccc4)c4ccccc4-5)cc32)cc1 ; c1cc(-c2ccc3c4ccccc4c4c(ccc5oc6ccccc6c54)c3c2)cc(N(c2ccc3c(c2)C2(c4ccccc4-c4ccccc42)c2ccccc2-3)c2ccc3c(c2)C2(c4ccccc4-c4ccccc42)c2ccccc2-3)c1]

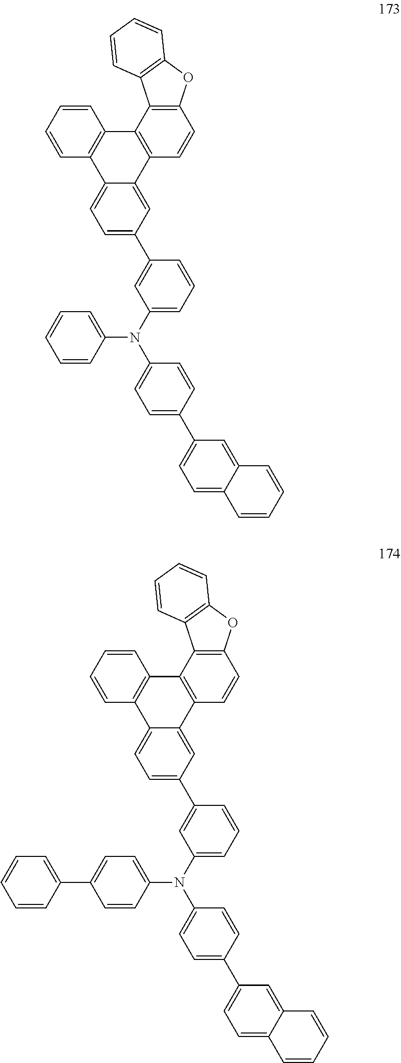 [SMILES: c1ccc(N(c2ccc(-c3ccc4ccccc4c3)cc2)c2cccc(-c3ccc4c5ccccc5c5c(ccc6oc7ccccc7c65)c4c3)c2)cc1 ; c1ccc(-c2ccc(N(c3ccc(-c4ccc5ccccc5c4)cc3)c3cccc(-c4ccc5c6ccccc6c6c(ccc7oc8ccccc8c76)c5c4)c3)cc2)cc1]

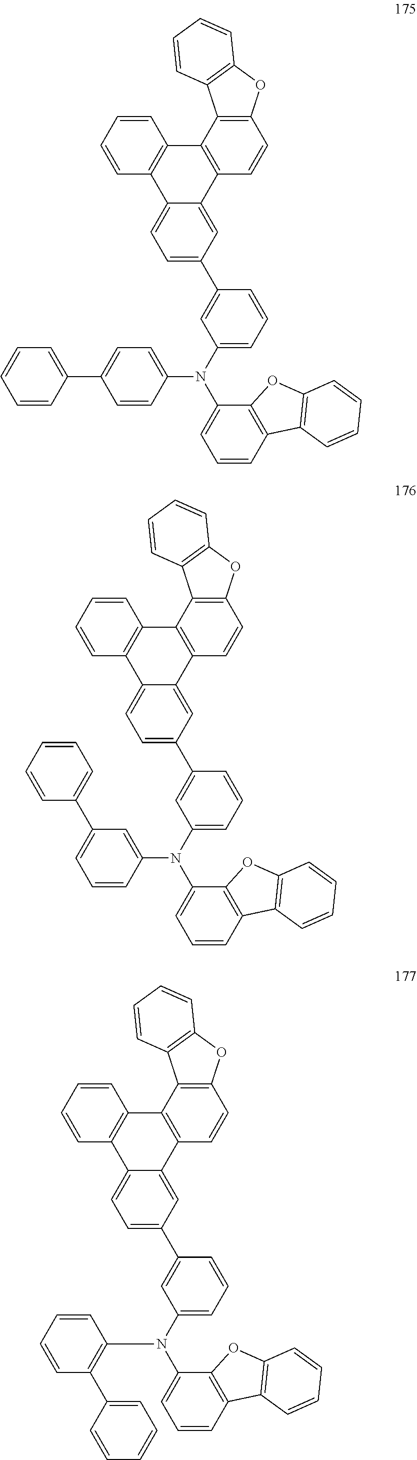 [SMILES: c1ccc(-c2ccc(N(c3cccc(-c4ccc5c6ccccc6c6c(ccc7oc8ccccc8c76)c5c4)c3)c3cccc4c3oc3ccccc34)cc2)cc1 ; c1ccc(-c2cccc(N(c3cccc(-c4ccc5c6ccccc6c6c(ccc7oc8ccccc8c76)c5c4)c3)c3cccc4c3oc3ccccc34)c2)cc1 ; c1ccc(-c2ccccc2N(c2cccc(-c3ccc4c5ccccc5c5c(ccc6oc7ccccc7c65)c4c3)c2)c2cccc3c2oc2ccccc23)cc1]

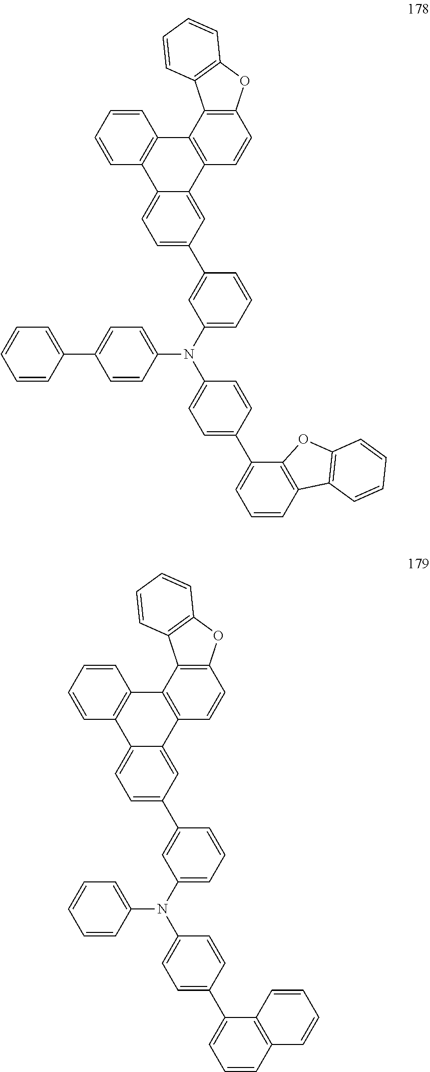 [SMILES: c1ccc(-c2ccc(N(c3ccc(-c4cccc5c4oc4ccccc45)cc3)c3cccc(-c4ccc5c6ccccc6c6c(ccc7oc8ccccc8c76)c5c4)c3)cc2)cc1 ; c1ccc(N(c2ccc(-c3cccc4ccccc34)cc2)c2cccc(-c3ccc4c5ccccc5c5c(ccc6oc7ccccc7c65)c4c3)c2)cc1]

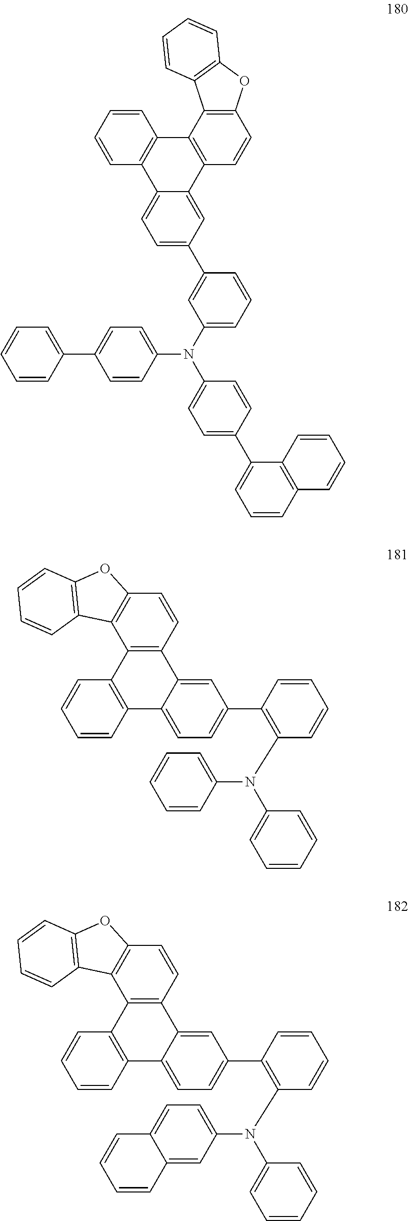 [SMILES: c1ccc(-c2ccc(N(c3ccc(-c4cccc5ccccc45)cc3)c3cccc(-c4ccc5c6ccccc6c6c(ccc7oc8ccccc8c76)c5c4)c3)cc2)cc1 ; c1ccc(N(c2ccccc2)c2ccccc2-c2ccc3c4ccccc4c4c(ccc5oc6ccccc6c54)c3c2)cc1 ; c1ccc(N(c2ccc3ccccc3c2)c2ccccc2-c2ccc3c4ccccc4c4c(ccc5oc6ccccc6c54)c3c2)cc1]

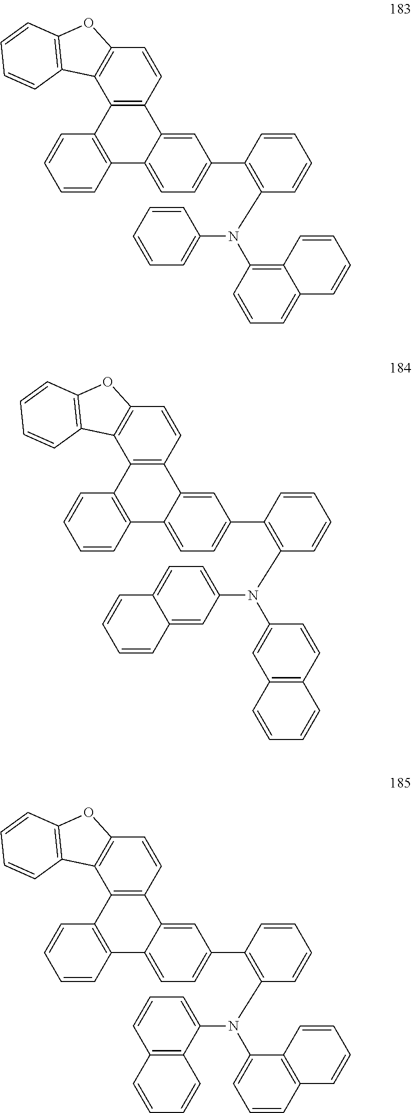 [SMILES: c1ccc(N(c2ccccc2-c2ccc3c4ccccc4c4c(ccc5oc6ccccc6c54)c3c2)c2cccc3ccccc23)cc1 ; c1ccc(N(c2ccc3ccccc3c2)c2ccc3ccccc3c2)c(-c2ccc3c4ccccc4c4c(ccc5oc6ccccc6c54)c3c2)c1 ; c1ccc(N(c2cccc3ccccc23)c2cccc3ccccc23)c(-c2ccc3c4ccccc4c4c(ccc5oc6ccccc6c54)c3c2)c1]

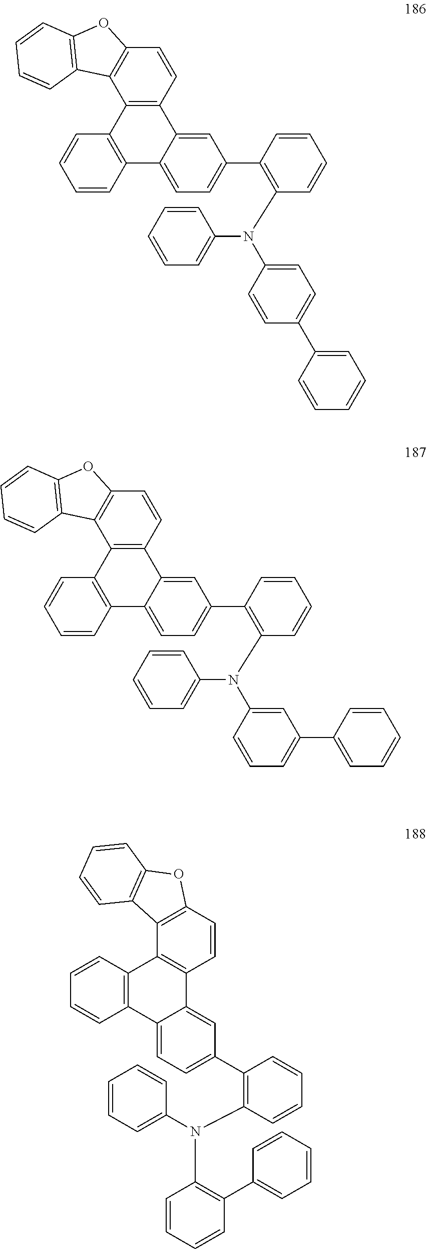 [SMILES: c1ccc(-c2ccc(N(c3ccccc3)c3ccccc3-c3ccc4c5ccccc5c5c(ccc6oc7ccccc7c65)c4c3)cc2)cc1 ; c1ccc(-c2cccc(N(c3ccccc3)c3ccccc3-c3ccc4c5ccccc5c5c(ccc6oc7ccccc7c65)c4c3)c2)cc1 ; c1ccc(-c2ccccc2N(c2ccccc2)c2ccccc2-c2ccc3c4ccccc4c4c(ccc5oc6ccccc6c54)c3c2)cc1]

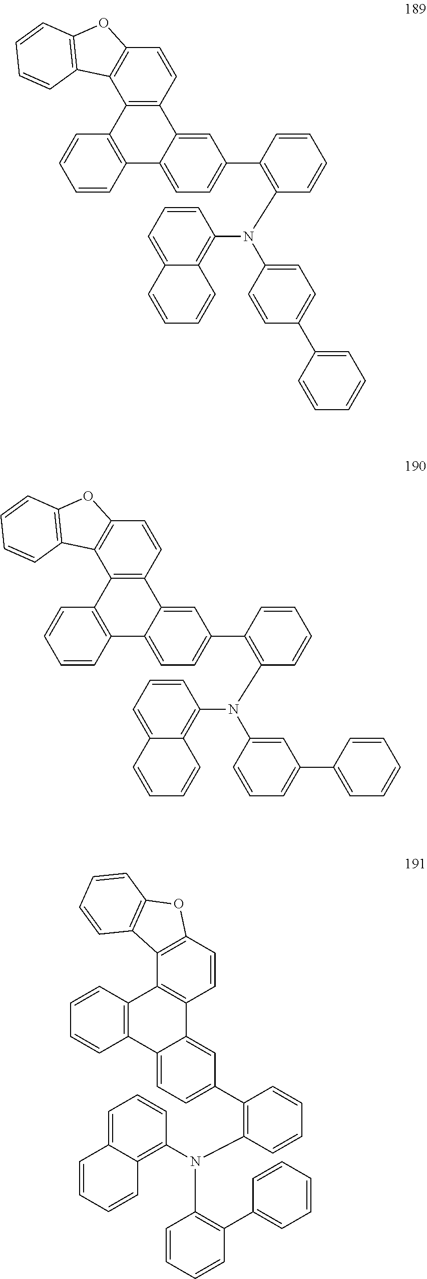 [SMILES: c1ccc(-c2ccc(N(c3ccccc3-c3ccc4c5ccccc5c5c(ccc6oc7ccccc7c65)c4c3)c3cccc4ccccc34)cc2)cc1 ; c1ccc(-c2cccc(N(c3ccccc3-c3ccc4c5ccccc5c5c(ccc6oc7ccccc7c65)c4c3)c3cccc4ccccc34)c2)cc1 ; c1ccc(-c2ccccc2N(c2ccccc2-c2ccc3c4ccccc4c4c(ccc5oc6ccccc6c54)c3c2)c2cccc3ccccc23)cc1]

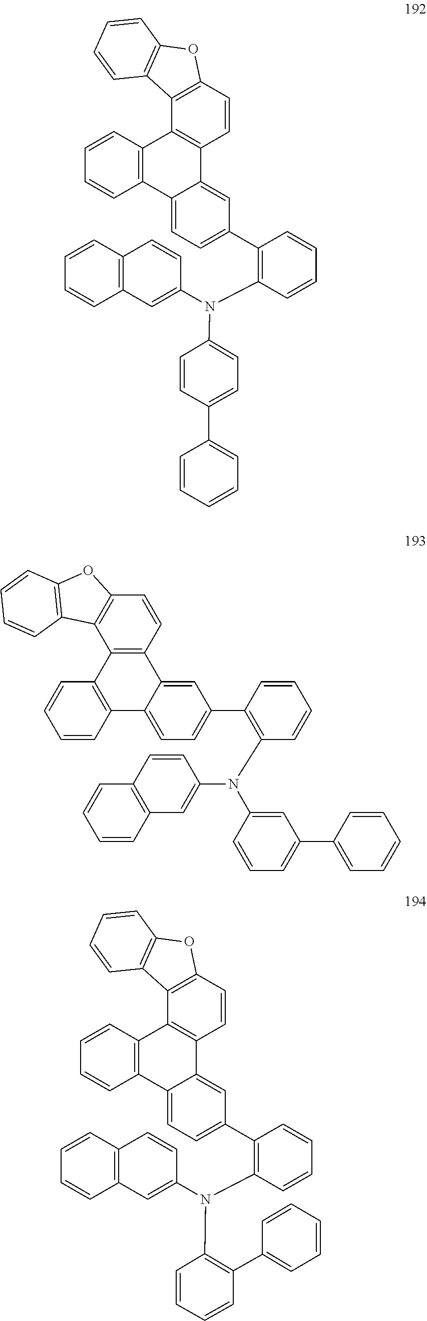 [SMILES: c1ccc(-c2ccc(N(c3ccc4ccccc4c3)c3ccccc3-c3ccc4c5ccccc5c5c(ccc6oc7ccccc7c65)c4c3)cc2)cc1 ; c1ccc(-c2cccc(N(c3ccc4ccccc4c3)c3ccccc3-c3ccc4c5ccccc5c5c(ccc6oc7ccccc7c65)c4c3)c2)cc1 ; c1ccc(-c2ccccc2N(c2ccc3ccccc3c2)c2ccccc2-c2ccc3c4ccccc4c4c(ccc5oc6ccccc6c54)c3c2)cc1]

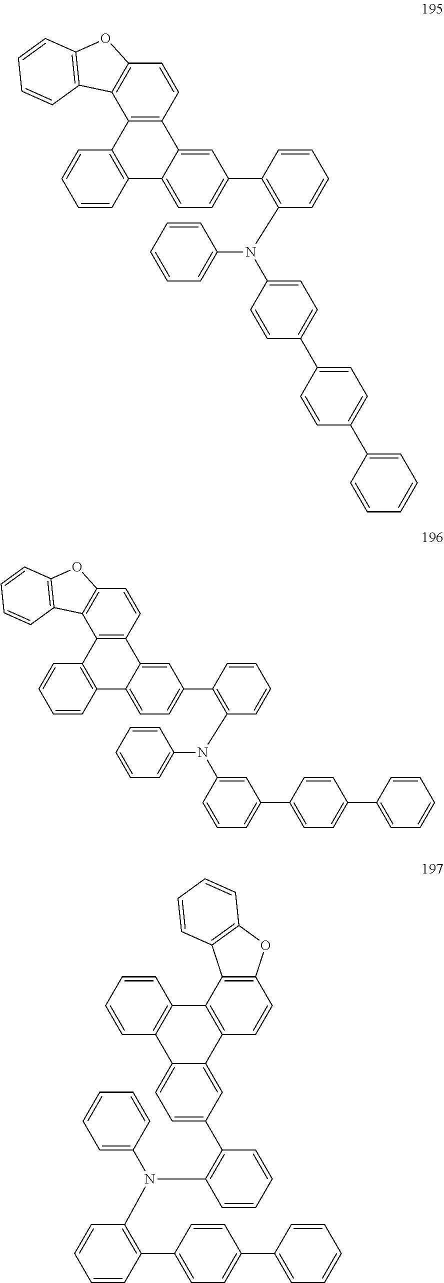 [SMILES: c1ccc(-c2ccc(-c3ccc(N(c4ccccc4)c4ccccc4-c4ccc5c6ccccc6c6c(ccc7oc8ccccc8c76)c5c4)cc3)cc2)cc1 ; c1ccc(-c2ccc(-c3cccc(N(c4ccccc4)c4ccccc4-c4ccc5c6ccccc6c6c(ccc7oc8ccccc8c76)c5c4)c3)cc2)cc1 ; c1ccc(-c2ccc(-c3ccccc3N(c3ccccc3)c3ccccc3-c3ccc4c5ccccc5c5c(ccc6oc7ccccc7c65)c4c3)cc2)cc1]

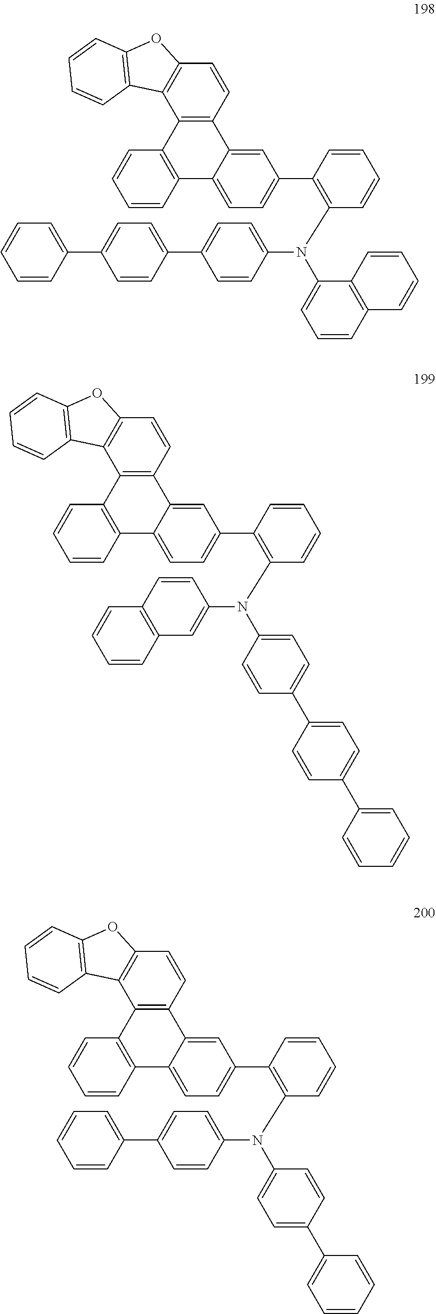 [SMILES: c1ccc(-c2ccc(-c3ccc(N(c4ccccc4-c4ccc5c6ccccc6c6c(ccc7oc8ccccc8c76)c5c4)c4cccc5ccccc45)cc3)cc2)cc1 ; c1ccc(-c2ccc(-c3ccc(N(c4ccc5ccccc5c4)c4ccccc4-c4ccc5c6ccccc6c6c(ccc7oc8ccccc8c76)c5c4)cc3)cc2)cc1 ; c1ccc(-c2ccc(N(c3ccc(-c4ccccc4)cc3)c3ccccc3-c3ccc4c5ccccc5c5c(ccc6oc7ccccc7c65)c4c3)cc2)cc1]

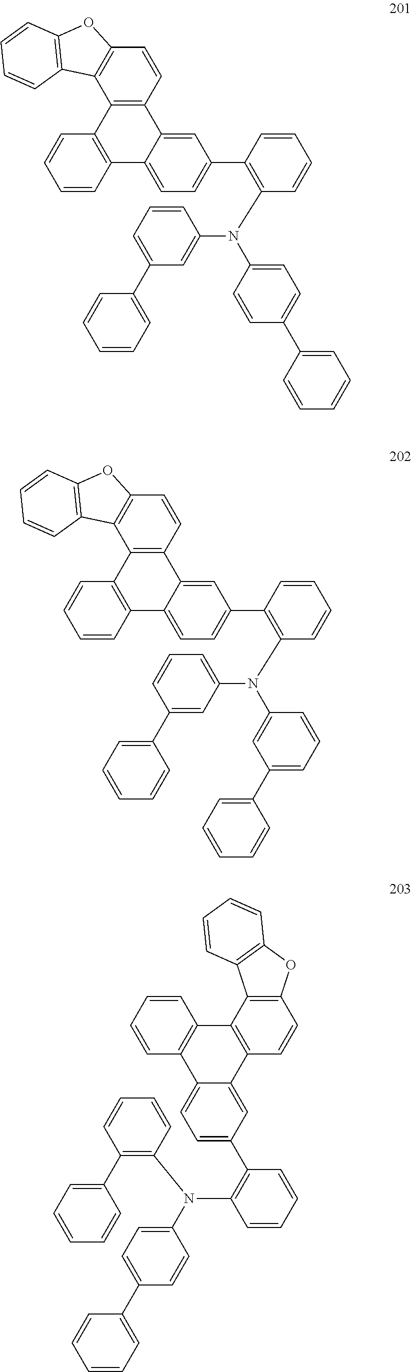

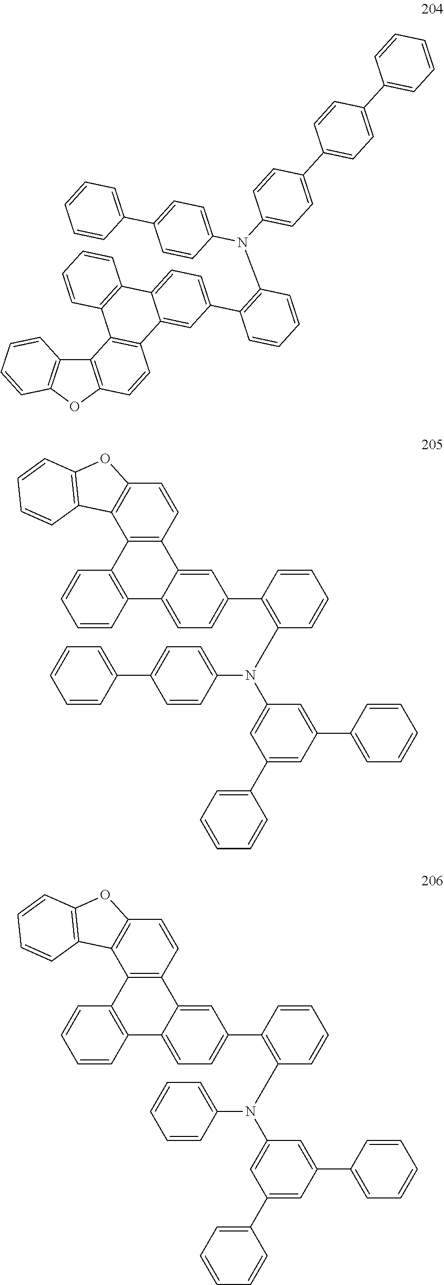 [SMILES: c1ccc(-c2ccc(-c3ccc(N(c4ccc(-c5ccccc5)cc4)c4ccccc4-c4ccc5c6ccccc6c6c(ccc7oc8ccccc8c76)c5c4)cc3)cc2)cc1 ; c1ccc(-c2ccc(N(c3cc(-c4ccccc4)cc(-c4ccccc4)c3)c3ccccc3-c3ccc4c5ccccc5c5c(ccc6oc7ccccc7c65)c4c3)cc2)cc1 ; c1ccc(-c2cc(-c3ccccc3)cc(N(c3ccccc3)c3ccccc3-c3ccc4c5ccccc5c5c(ccc6oc7ccccc7c65)c4c3)c2)cc1]

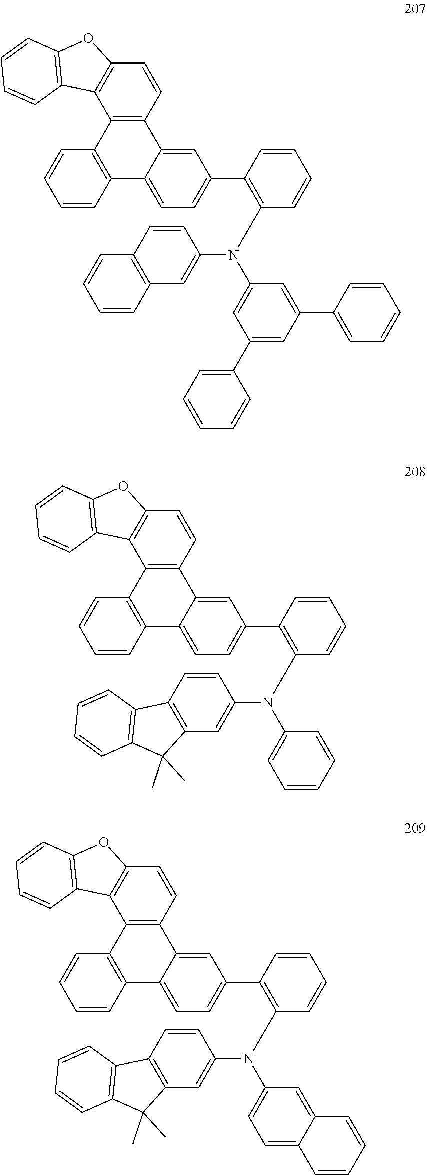 [SMILES: c1ccc(-c2cc(-c3ccccc3)cc(N(c3ccc4ccccc4c3)c3ccccc3-c3ccc4c5ccccc5c5c(ccc6oc7ccccc7c65)c4c3)c2)cc1 ; CC1(C)c2ccccc2-c2ccc(N(c3ccccc3)c3ccccc3-c3ccc4c5ccccc5c5c(ccc6oc7ccccc7c65)c4c3)cc21 ; CC1(C)c2ccccc2-c2ccc(N(c3ccc4ccccc4c3)c3ccccc3-c3ccc4c5ccccc5c5c(ccc6oc7ccccc7c65)c4c3)cc21]

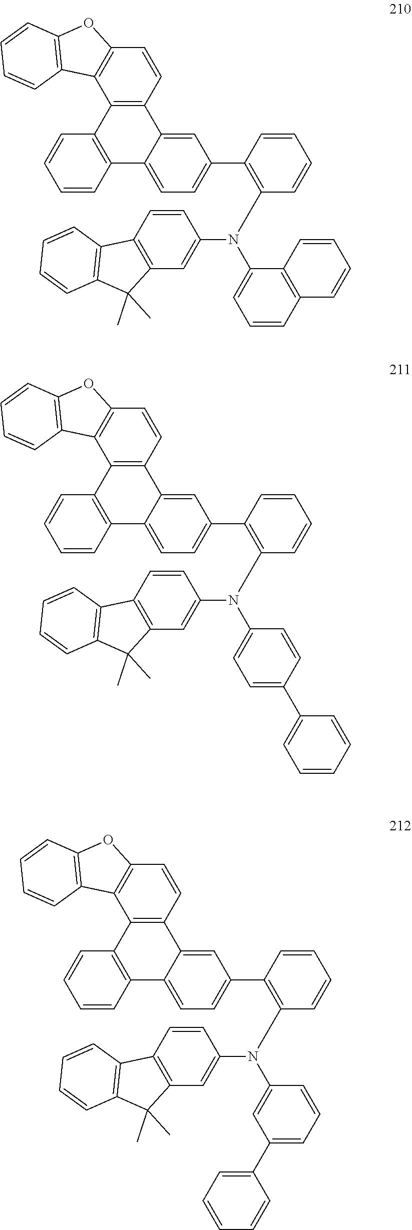 [SMILES: CC1(C)c2ccccc2-c2ccc(N(c3ccccc3-c3ccc4c5ccccc5c5c(ccc6oc7ccccc7c65)c4c3)c3cccc4ccccc34)cc21 ; CC1(C)c2ccccc2-c2ccc(N(c3ccc(-c4ccccc4)cc3)c3ccccc3-c3ccc4c5ccccc5c5c(ccc6oc7ccccc7c65)c4c3)cc21 ; CC1(C)c2ccccc2-c2ccc(N(c3cccc(-c4ccccc4)c3)c3ccccc3-c3ccc4c5ccccc5c5c(ccc6oc7ccccc7c65)c4c3)cc21]

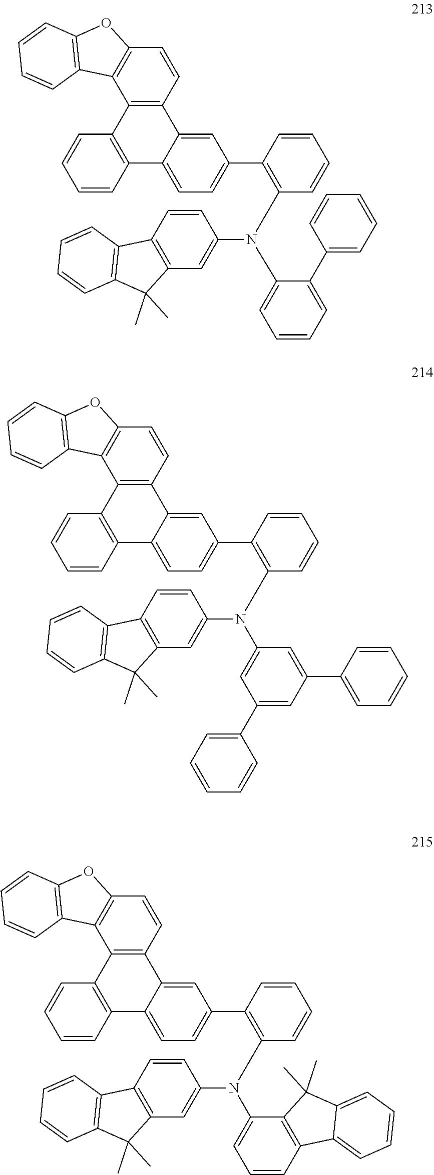 [SMILES: CC1(C)c2ccccc2-c2ccc(N(c3ccccc3-c3ccccc3)c3ccccc3-c3ccc4c5ccccc5c5c(ccc6oc7ccccc7c65)c4c3)cc21 ; CC1(C)c2ccccc2-c2ccc(N(c3cc(-c4ccccc4)cc(-c4ccccc4)c3)c3ccccc3-c3ccc4c5ccccc5c5c(ccc6oc7ccccc7c65)c4c3)cc21 ; CC1(C)c2ccccc2-c2ccc(N(c3ccccc3-c3ccc4c5ccccc5c5c(ccc6oc7ccccc7c65)c4c3)c3cccc4c3C(C)(C)c3ccccc3-4)cc21]

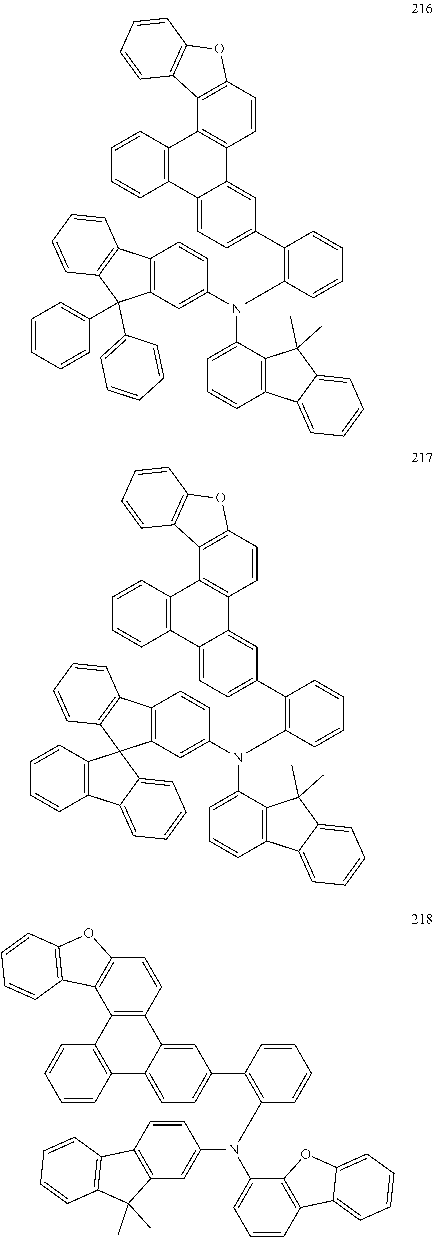 [SMILES: CC1(C)c2ccccc2-c2cccc(N(c3ccc4c(c3)C(c3ccccc3)(c3ccccc3)c3ccccc3-4)c3ccccc3-c3ccc4c5ccccc5c5c(ccc6oc7ccccc7c65)c4c3)c21 ; CC1(C)c2ccccc2-c2cccc(N(c3ccc4c(c3)C3(c5ccccc5-c5ccccc53)c3ccccc3-4)c3ccccc3-c3ccc4c5ccccc5c5c(ccc6oc7ccccc7c65)c4c3)c21 ; CC1(C)c2ccccc2-c2ccc(N(c3ccccc3-c3ccc4c5ccccc5c5c(ccc6oc7ccccc7c65)c4c3)c3cccc4c3oc3ccccc34)cc21]

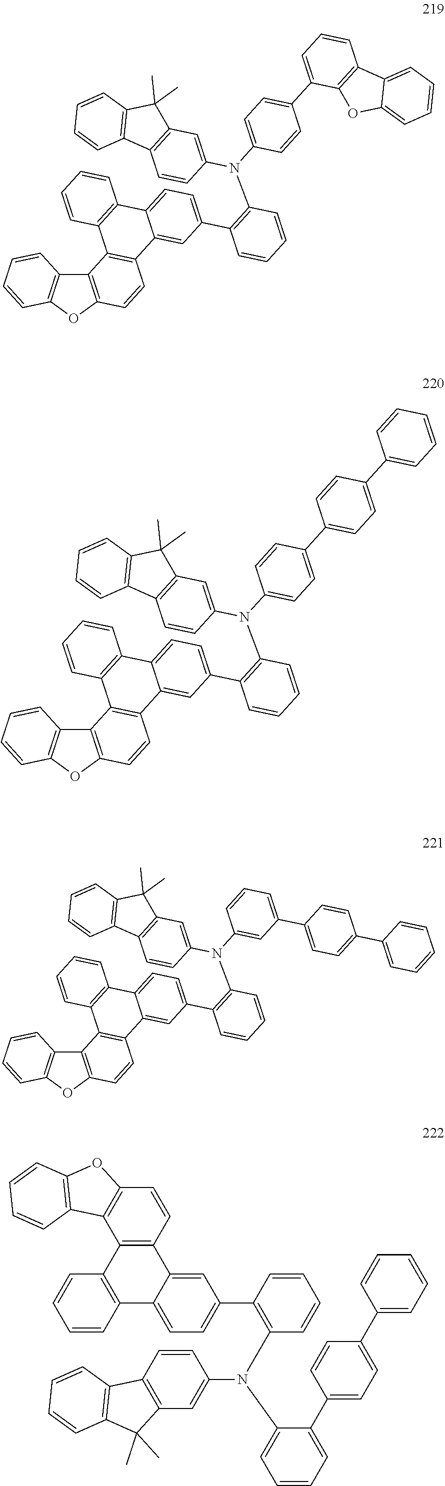 [SMILES: CC1(C)c2ccccc2-c2ccc(N(c3ccc(-c4cccc5c4oc4ccccc45)cc3)c3ccccc3-c3ccc4c5ccccc5c5c(ccc6oc7ccccc7c65)c4c3)cc21 ; CC1(C)c2ccccc2-c2ccc(N(c3ccc(-c4ccc(-c5ccccc5)cc4)cc3)c3ccccc3-c3ccc4c5ccccc5c5c(ccc6oc7ccccc7c65)c4c3)cc21 ; CC1(C)c2ccccc2-c2ccc(N(c3cccc(-c4ccc(-c5ccccc5)cc4)c3)c3ccccc3-c3ccc4c5ccccc5c5c(ccc6oc7ccccc7c65)c4c3)cc21 ; CC1(C)c2ccccc2-c2ccc(N(c3ccccc3-c3ccc(-c4ccccc4)cc3)c3ccccc3-c3ccc4c5ccccc5c5c(ccc6oc7ccccc7c65)c4c3)cc21]

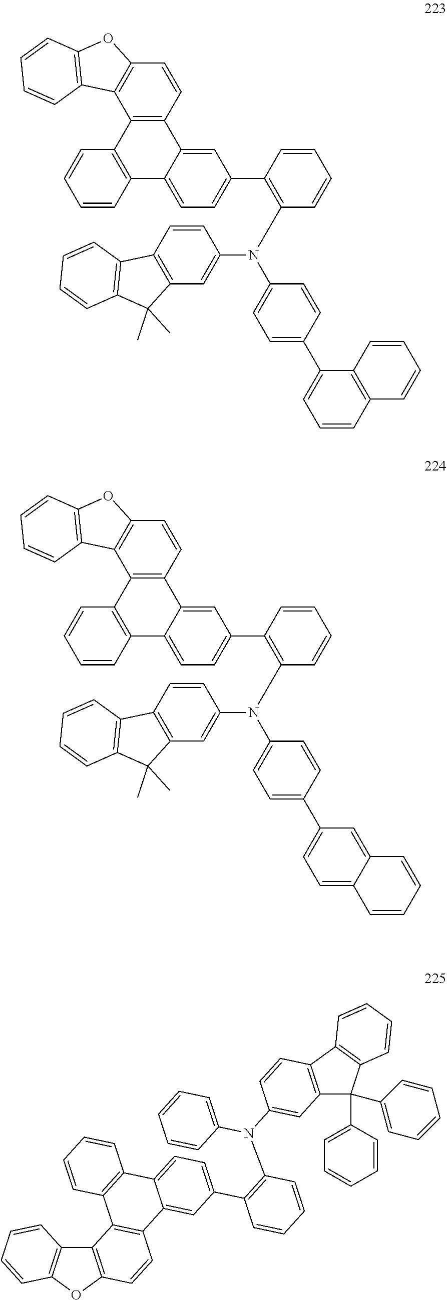 [SMILES: CC1(C)c2ccccc2-c2ccc(N(c3ccc(-c4cccc5ccccc45)cc3)c3ccccc3-c3ccc4c5ccccc5c5c(ccc6oc7ccccc7c65)c4c3)cc21 ; CC1(C)c2ccccc2-c2ccc(N(c3ccc(-c4ccc5ccccc5c4)cc3)c3ccccc3-c3ccc4c5ccccc5c5c(ccc6oc7ccccc7c65)c4c3)cc21 ; c1ccc(N(c2ccc3c(c2)C(c2ccccc2)(c2ccccc2)c2ccccc2-3)c2ccccc2-c2ccc3c4ccccc4c4c(ccc5oc6ccccc6c54)c3c2)cc1]

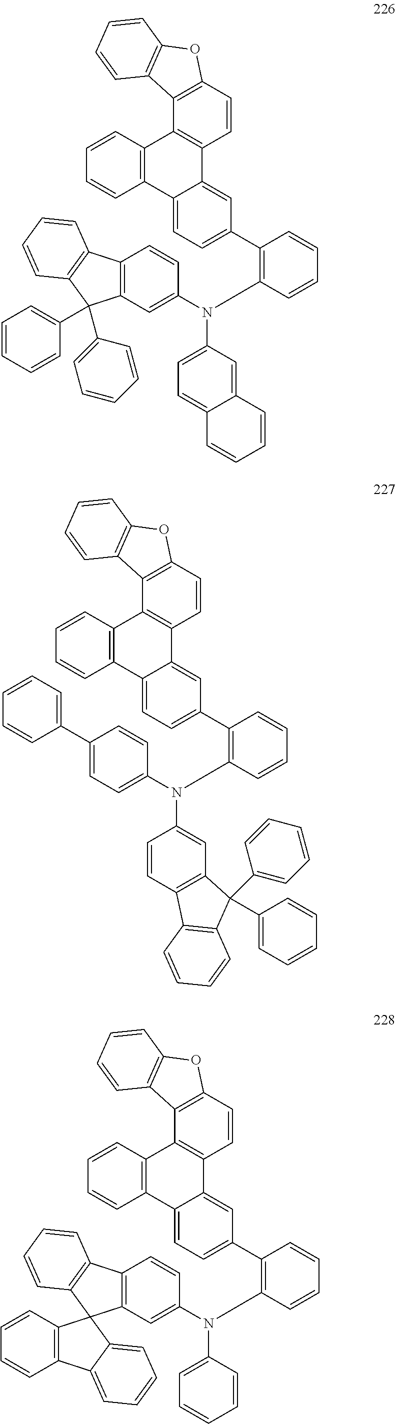 [SMILES: c1ccc(C2(c3ccccc3)c3ccccc3-c3ccc(N(c4ccc5ccccc5c4)c4ccccc4-c4ccc5c6ccccc6c6c(ccc7oc8ccccc8c76)c5c4)cc32)cc1 ; c1ccc(-c2ccc(N(c3ccc4c(c3)C(c3ccccc3)(c3ccccc3)c3ccccc3-4)c3ccccc3-c3ccc4c5ccccc5c5c(ccc6oc7ccccc7c65)c4c3)cc2)cc1 ; c1ccc(N(c2ccc3c(c2)C2(c4ccccc4-c4ccccc42)c2ccccc2-3)c2ccccc2-c2ccc3c4ccccc4c4c(ccc5oc6ccccc6c54)c3c2)cc1]

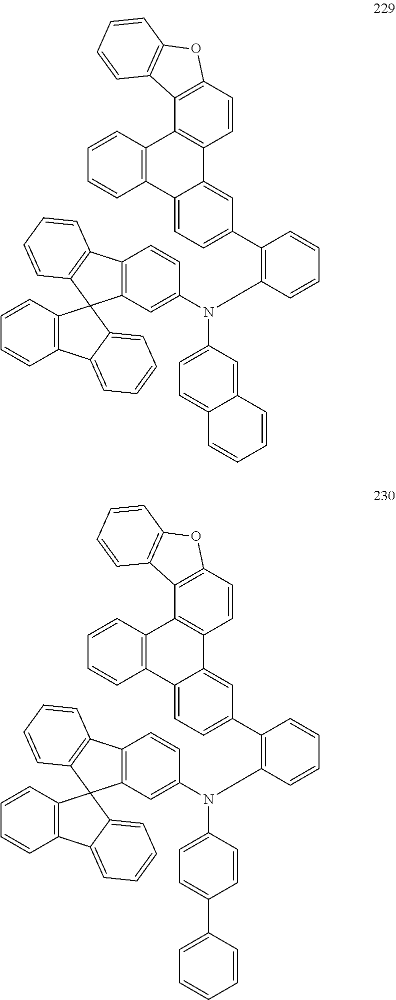 [SMILES: c1ccc(N(c2ccc3c(c2)C2(c4ccccc4-c4ccccc42)c2ccccc2-3)c2ccc3ccccc3c2)c(-c2ccc3c4ccccc4c4c(ccc5oc6ccccc6c54)c3c2)c1 ; c1ccc(-c2ccc(N(c3ccc4c(c3)C3(c5ccccc5-c5ccccc53)c3ccccc3-4)c3ccccc3-c3ccc4c5ccccc5c5c(ccc6oc7ccccc7c65)c4c3)cc2)cc1]

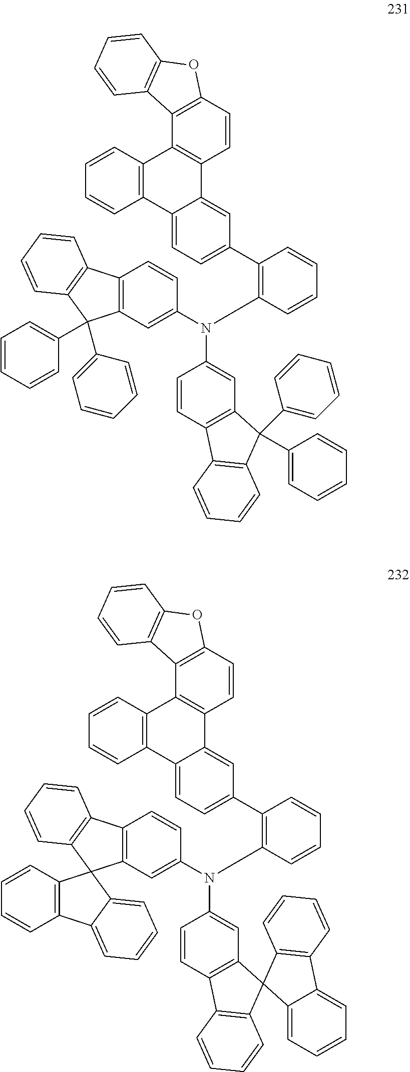 [SMILES: c1ccc(C2(c3ccccc3)c3ccccc3-c3ccc(N(c4ccc5c(c4)C(c4ccccc4)(c4ccccc4)c4ccccc4-5)c4ccccc4-c4ccc5c6ccccc6c6c(ccc7oc8ccccc8c76)c5c4)cc32)cc1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3c4ccccc4c4c(ccc5oc6ccccc6c54)c3c2)c1]

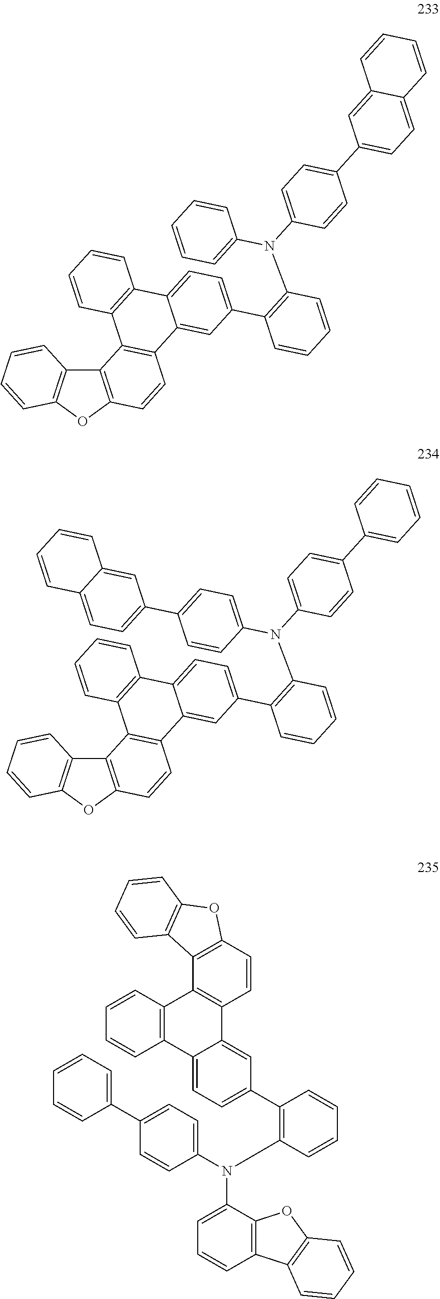 [SMILES: c1ccc(N(c2ccc(-c3ccc4ccccc4c3)cc2)c2ccccc2-c2ccc3c4ccccc4c4c(ccc5oc6ccccc6c54)c3c2)cc1 ; c1ccc(-c2ccc(N(c3ccc(-c4ccc5ccccc5c4)cc3)c3ccccc3-c3ccc4c5ccccc5c5c(ccc6oc7ccccc7c65)c4c3)cc2)cc1 ; c1ccc(-c2ccc(N(c3ccccc3-c3ccc4c5ccccc5c5c(ccc6oc7ccccc7c65)c4c3)c3cccc4c3oc3ccccc34)cc2)cc1]

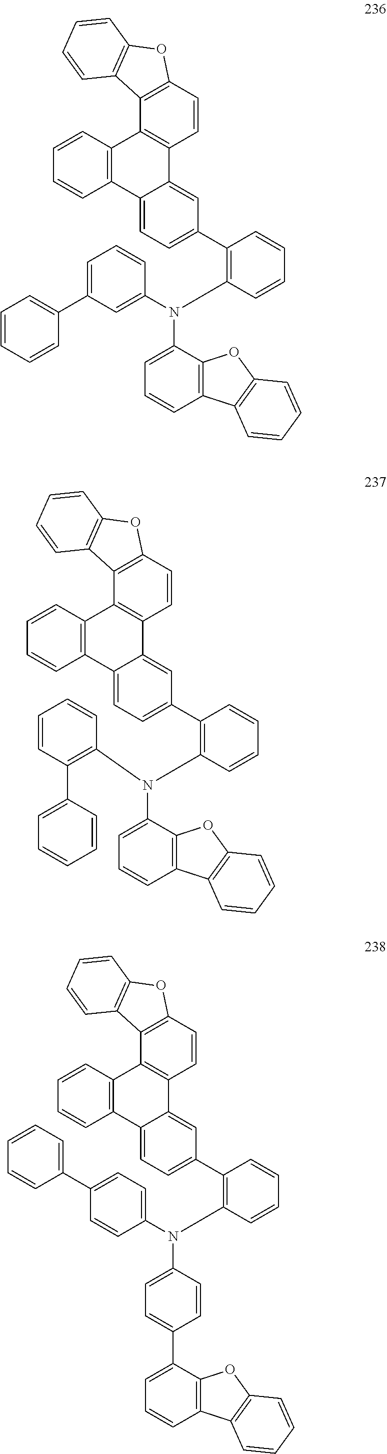 [SMILES: c1ccc(-c2cccc(N(c3ccccc3-c3ccc4c5ccccc5c5c(ccc6oc7ccccc7c65)c4c3)c3cccc4c3oc3ccccc34)c2)cc1 ; c1ccc(-c2ccccc2N(c2ccccc2-c2ccc3c4ccccc4c4c(ccc5oc6ccccc6c54)c3c2)c2cccc3c2oc2ccccc23)cc1 ; c1ccc(-c2ccc(N(c3ccc(-c4cccc5c4oc4ccccc45)cc3)c3ccccc3-c3ccc4c5ccccc5c5c(ccc6oc7ccccc7c65)c4c3)cc2)cc1]

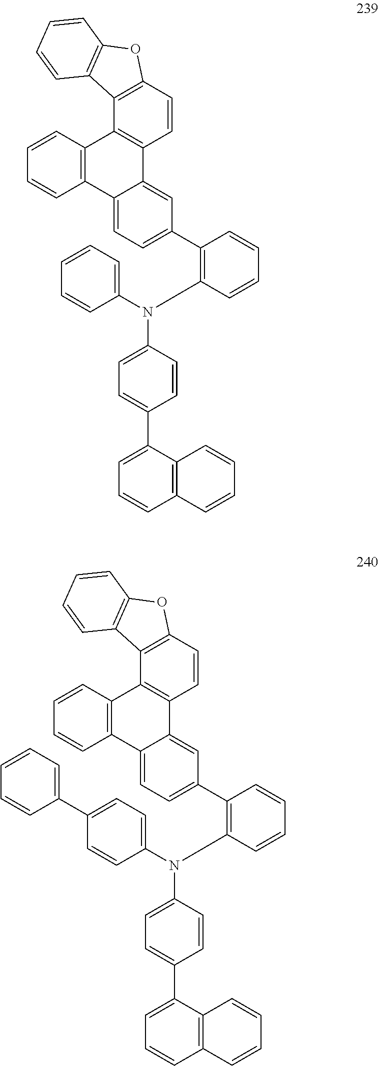 [SMILES: c1ccc(N(c2ccc(-c3cccc4ccccc34)cc2)c2ccccc2-c2ccc3c4ccccc4c4c(ccc5oc6ccccc6c54)c3c2)cc1 ; c1ccc(-c2ccc(N(c3ccc(-c4cccc5ccccc45)cc3)c3ccccc3-c3ccc4c5ccccc5c5c(ccc6oc7ccccc7c65)c4c3)cc2)cc1]

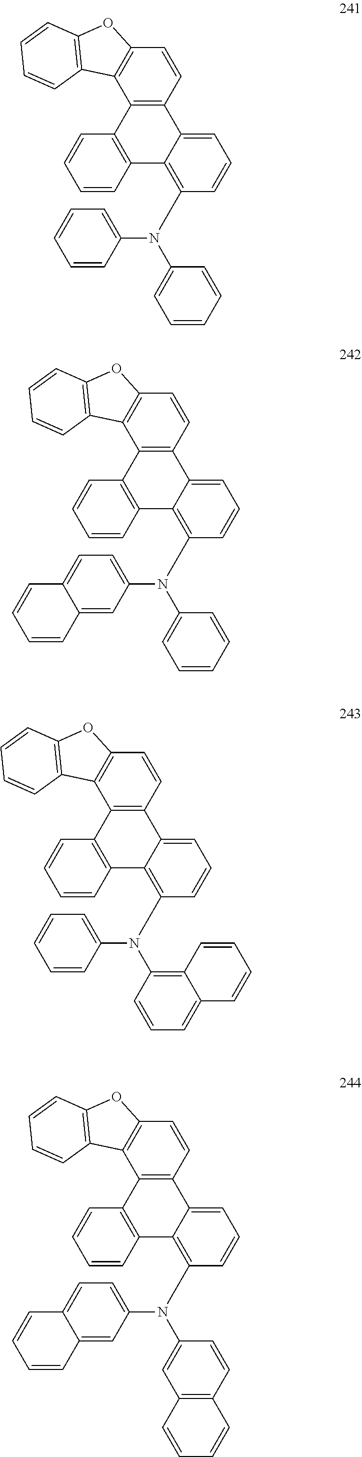 [SMILES: c1ccc(N(c2ccccc2)c2cccc3c4ccc5oc6ccccc6c5c4c4ccccc4c23)cc1 ; c1ccc(N(c2ccc3ccccc3c2)c2cccc3c4ccc5oc6ccccc6c5c4c4ccccc4c23)cc1 ; c1ccc(N(c2cccc3ccccc23)c2cccc3c4ccc5oc6ccccc6c5c4c4ccccc4c23)cc1 ; c1ccc2cc(N(c3ccc4ccccc4c3)c3cccc4c5ccc6oc7ccccc7c6c5c5ccccc5c34)ccc2c1]

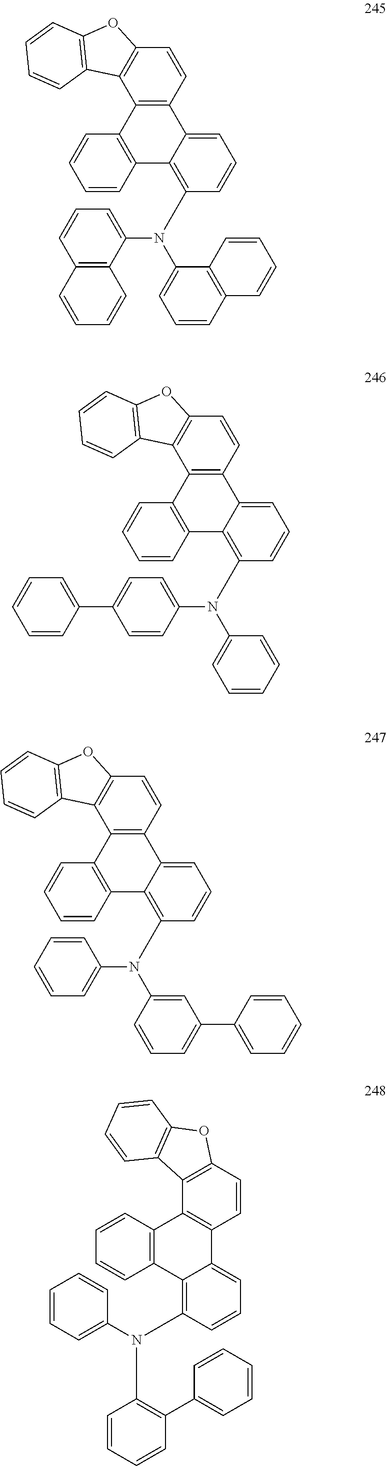 [SMILES: c1ccc2c(N(c3cccc4ccccc34)c3cccc4c5ccc6oc7ccccc7c6c5c5ccccc5c34)cccc2c1 ; c1ccc(-c2ccc(N(c3ccccc3)c3cccc4c5ccc6oc7ccccc7c6c5c5ccccc5c34)cc2)cc1 ; c1ccc(-c2cccc(N(c3ccccc3)c3cccc4c5ccc6oc7ccccc7c6c5c5ccccc5c34)c2)cc1 ; c1ccc(-c2ccccc2N(c2ccccc2)c2cccc3c4ccc5oc6ccccc6c5c4c4ccccc4c23)cc1]

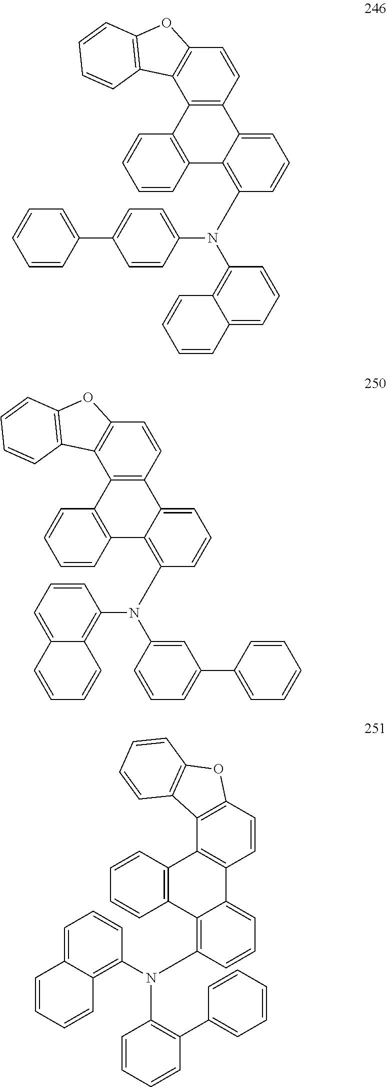

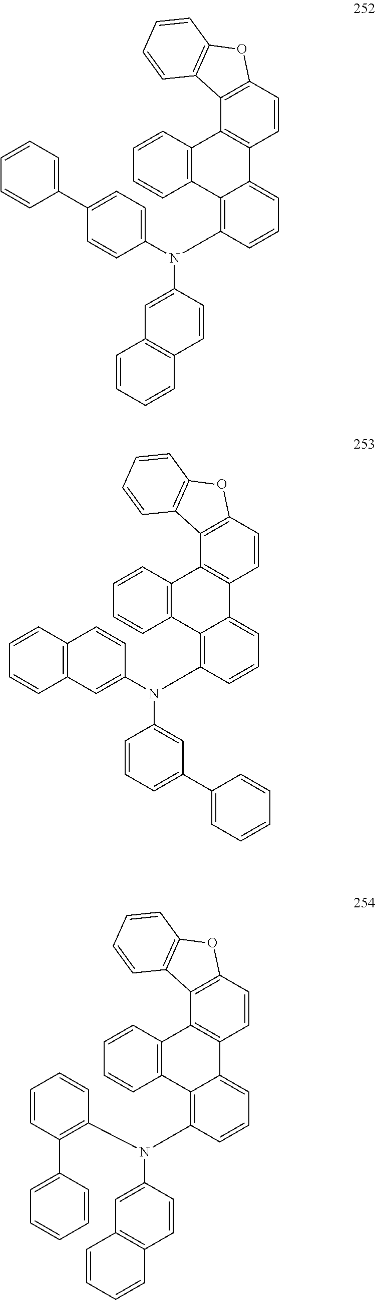 [SMILES: c1ccc(-c2ccc(N(c3ccc4ccccc4c3)c3cccc4c5ccc6oc7ccccc7c6c5c5ccccc5c34)cc2)cc1 ; c1ccc(-c2cccc(N(c3ccc4ccccc4c3)c3cccc4c5ccc6oc7ccccc7c6c5c5ccccc5c34)c2)cc1 ; c1ccc(-c2ccccc2N(c2ccc3ccccc3c2)c2cccc3c4ccc5oc6ccccc6c5c4c4ccccc4c23)cc1]

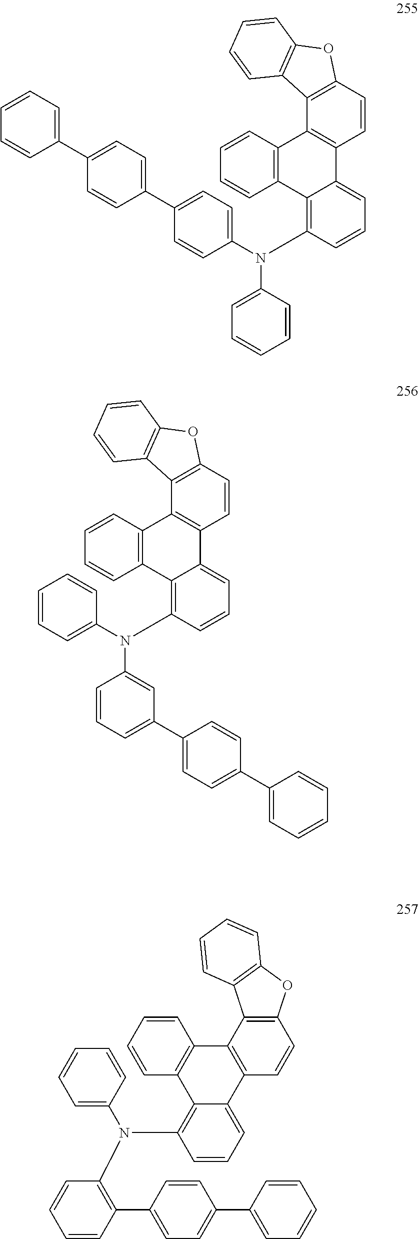 [SMILES: c1ccc(-c2ccc(-c3ccc(N(c4ccccc4)c4cccc5c6ccc7oc8ccccc8c7c6c6ccccc6c45)cc3)cc2)cc1 ; c1ccc(-c2ccc(-c3cccc(N(c4ccccc4)c4cccc5c6ccc7oc8ccccc8c7c6c6ccccc6c45)c3)cc2)cc1 ; c1ccc(-c2ccc(-c3ccccc3N(c3ccccc3)c3cccc4c5ccc6oc7ccccc7c6c5c5ccccc5c34)cc2)cc1]

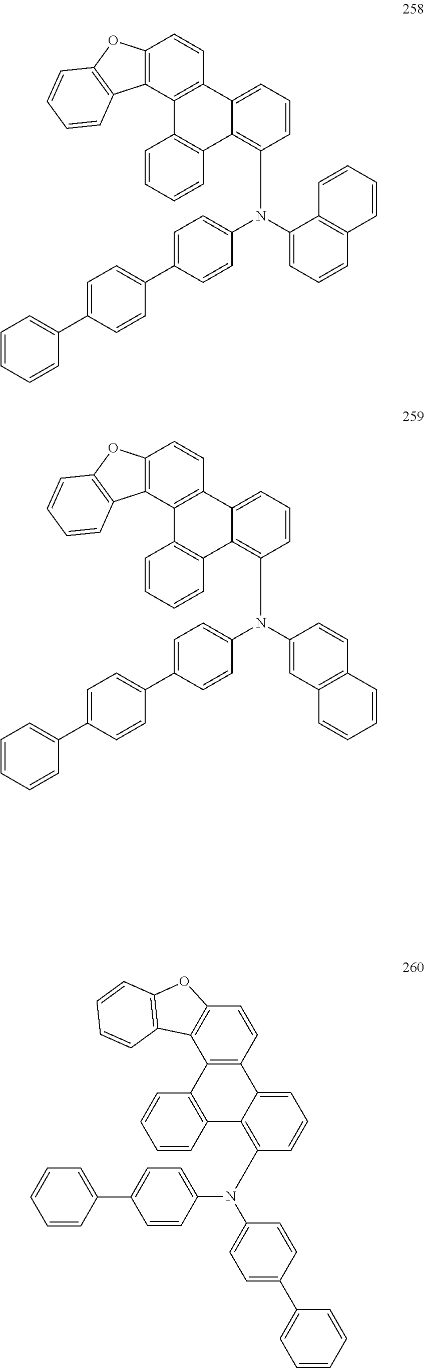 [SMILES: c1ccc(-c2ccc(-c3ccc(N(c4cccc5ccccc45)c4cccc5c6ccc7oc8ccccc8c7c6c6ccccc6c45)cc3)cc2)cc1 ; c1ccc(-c2ccc(-c3ccc(N(c4ccc5ccccc5c4)c4cccc5c6ccc7oc8ccccc8c7c6c6ccccc6c45)cc3)cc2)cc1 ; c1ccc(-c2ccc(N(c3ccc(-c4ccccc4)cc3)c3cccc4c5ccc6oc7ccccc7c6c5c5ccccc5c34)cc2)cc1]

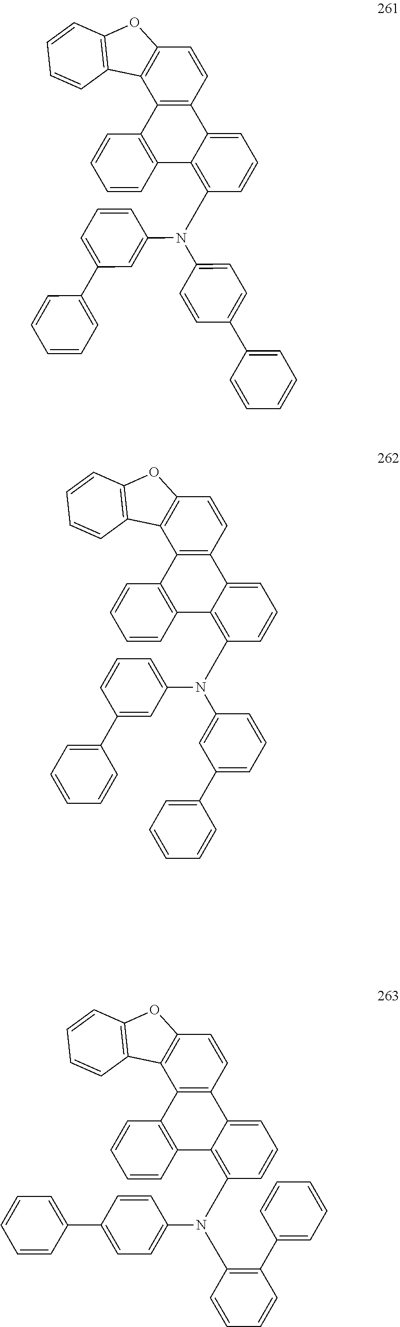 [SMILES: c1ccc(-c2ccc(N(c3cccc(-c4ccccc4)c3)c3cccc4c5ccc6oc7ccccc7c6c5c5ccccc5c34)cc2)cc1 ; c1ccc(-c2cccc(N(c3cccc(-c4ccccc4)c3)c3cccc4c5ccc6oc7ccccc7c6c5c5ccccc5c34)c2)cc1 ; c1ccc(-c2ccc(N(c3ccccc3-c3ccccc3)c3cccc4c5ccc6oc7ccccc7c6c5c5ccccc5c34)cc2)cc1]

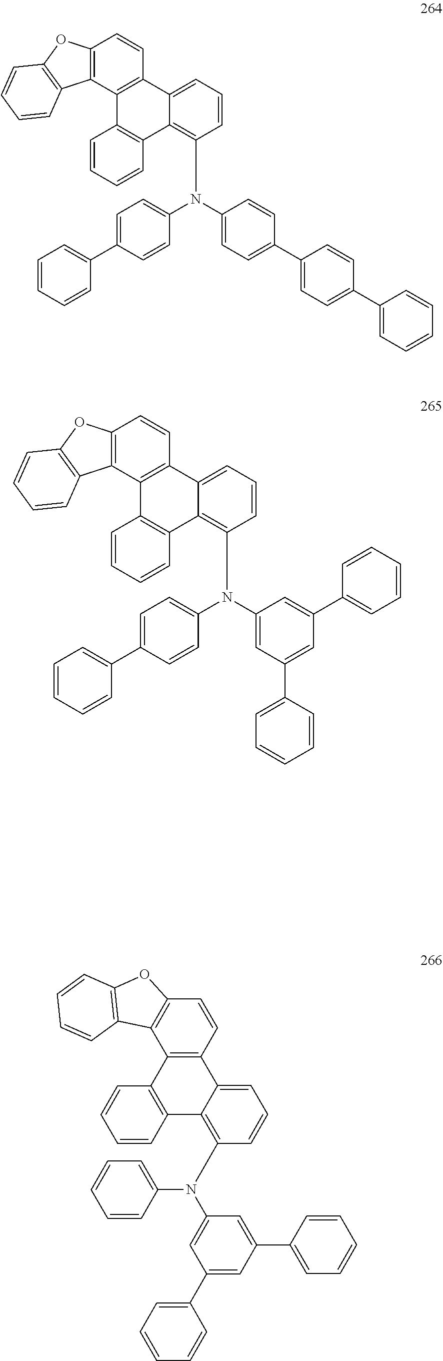 [SMILES: c1ccc(-c2ccc(-c3ccc(N(c4ccc(-c5ccccc5)cc4)c4cccc5c6ccc7oc8ccccc8c7c6c6ccccc6c45)cc3)cc2)cc1 ; c1ccc(-c2ccc(N(c3cc(-c4ccccc4)cc(-c4ccccc4)c3)c3cccc4c5ccc6oc7ccccc7c6c5c5ccccc5c34)cc2)cc1 ; c1ccc(-c2cc(-c3ccccc3)cc(N(c3ccccc3)c3cccc4c5ccc6oc7ccccc7c6c5c5ccccc5c34)c2)cc1]

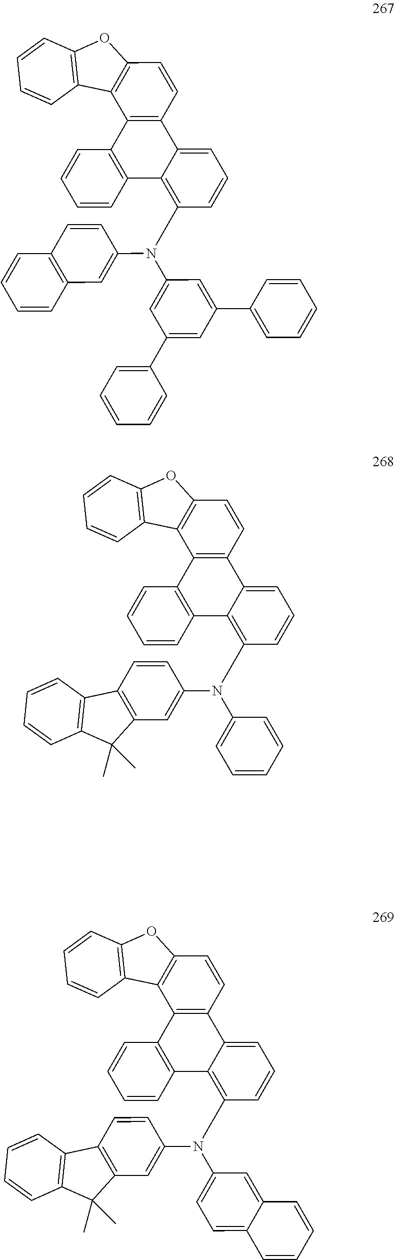 [SMILES: c1ccc(-c2cc(-c3ccccc3)cc(N(c3ccc4ccccc4c3)c3cccc4c5ccc6oc7ccccc7c6c5c5ccccc5c34)c2)cc1 ; CC1(C)c2ccccc2-c2ccc(N(c3ccccc3)c3cccc4c5ccc6oc7ccccc7c6c5c5ccccc5c34)cc21 ; CC1(C)c2ccccc2-c2ccc(N(c3ccc4ccccc4c3)c3cccc4c5ccc6oc7ccccc7c6c5c5ccccc5c34)cc21]

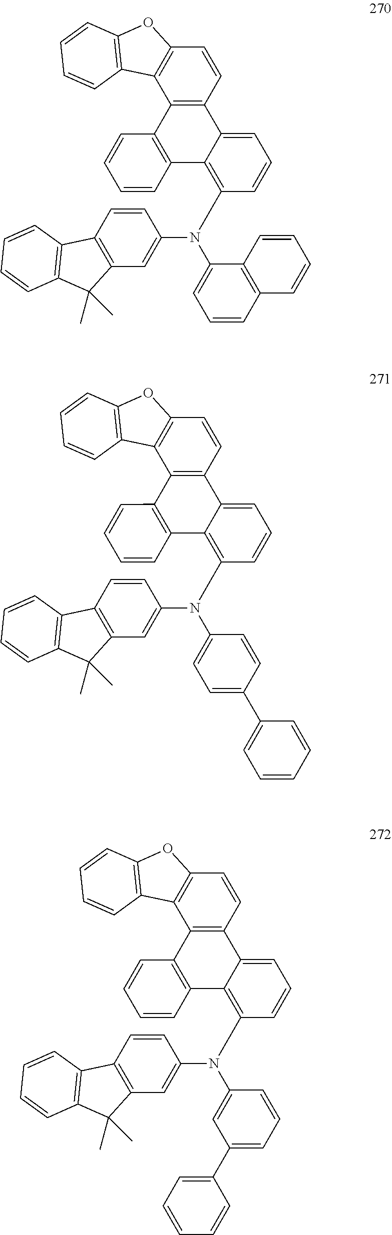 [SMILES: CC1(C)c2ccccc2-c2ccc(N(c3cccc4ccccc34)c3cccc4c5ccc6oc7ccccc7c6c5c5ccccc5c34)cc21 ; CC1(C)c2ccccc2-c2ccc(N(c3ccc(-c4ccccc4)cc3)c3cccc4c5ccc6oc7ccccc7c6c5c5ccccc5c34)cc21 ; CC1(C)c2ccccc2-c2ccc(N(c3cccc(-c4ccccc4)c3)c3cccc4c5ccc6oc7ccccc7c6c5c5ccccc5c34)cc21]

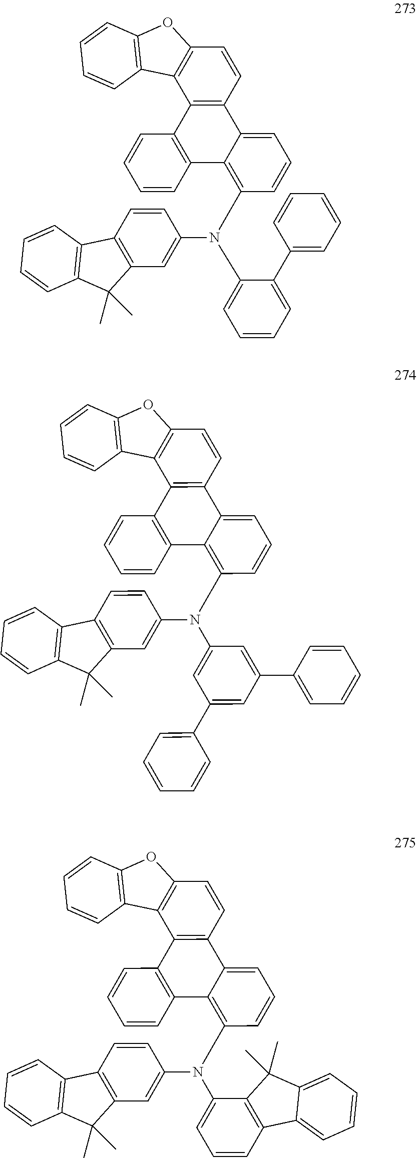 [SMILES: CC1(C)c2ccccc2-c2ccc(N(c3ccccc3-c3ccccc3)c3cccc4c5ccc6oc7ccccc7c6c5c5ccccc5c34)cc21 ; CC1(C)c2ccccc2-c2ccc(N(c3cc(-c4ccccc4)cc(-c4ccccc4)c3)c3cccc4c5ccc6oc7ccccc7c6c5c5ccccc5c34)cc21 ; CC1(C)c2ccccc2-c2ccc(N(c3cccc4c3C(C)(C)c3ccccc3-4)c3cccc4c5ccc6oc7ccccc7c6c5c5ccccc5c34)cc21]

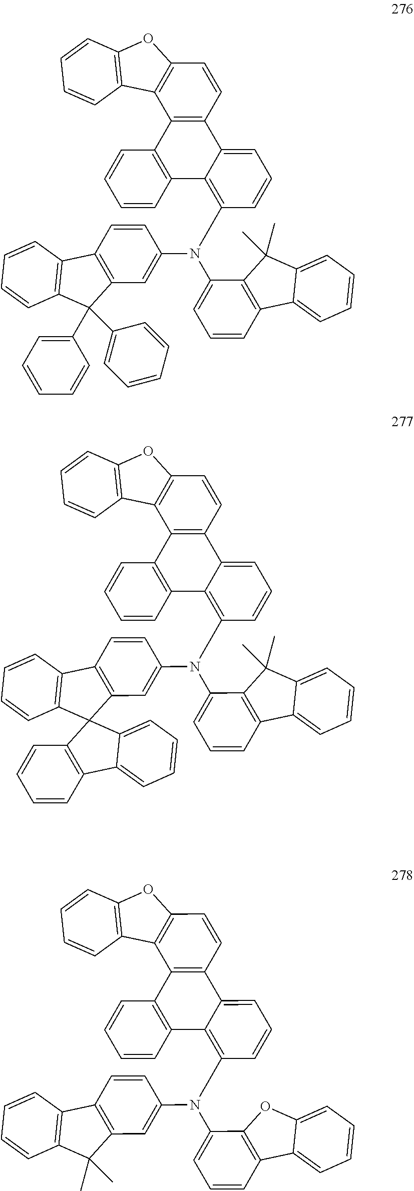 [SMILES: CC1(C)c2ccccc2-c2cccc(N(c3ccc4c(c3)C(c3ccccc3)(c3ccccc3)c3ccccc3-4)c3cccc4c5ccc6oc7ccccc7c6c5c5ccccc5c34)c21 ; CC1(C)c2ccccc2-c2cccc(N(c3ccc4c(c3)C3(c5ccccc5-c5ccccc53)c3ccccc3-4)c3cccc4c5ccc6oc7ccccc7c6c5c5ccccc5c34)c21 ; CC1(C)c2ccccc2-c2ccc(N(c3cccc4c3oc3ccccc34)c3cccc4c5ccc6oc7ccccc7c6c5c5ccccc5c34)cc21]

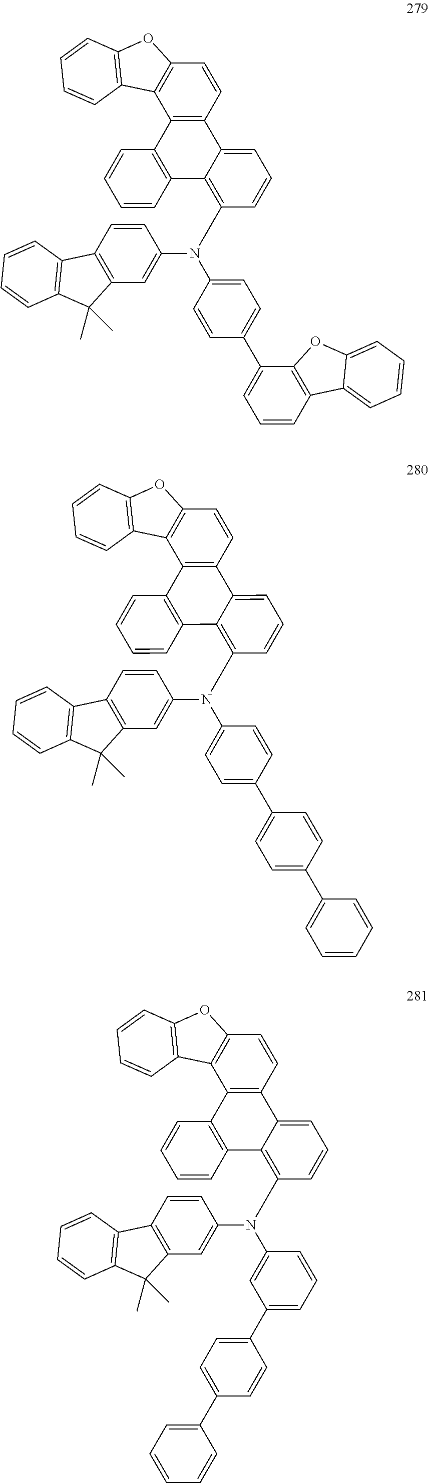 [SMILES: CC1(C)c2ccccc2-c2ccc(N(c3ccc(-c4cccc5c4oc4ccccc45)cc3)c3cccc4c5ccc6oc7ccccc7c6c5c5ccccc5c34)cc21 ; CC1(C)c2ccccc2-c2ccc(N(c3ccc(-c4ccc(-c5ccccc5)cc4)cc3)c3cccc4c5ccc6oc7ccccc7c6c5c5ccccc5c34)cc21 ; CC1(C)c2ccccc2-c2ccc(N(c3cccc(-c4ccc(-c5ccccc5)cc4)c3)c3cccc4c5ccc6oc7ccccc7c6c5c5ccccc5c34)cc21]

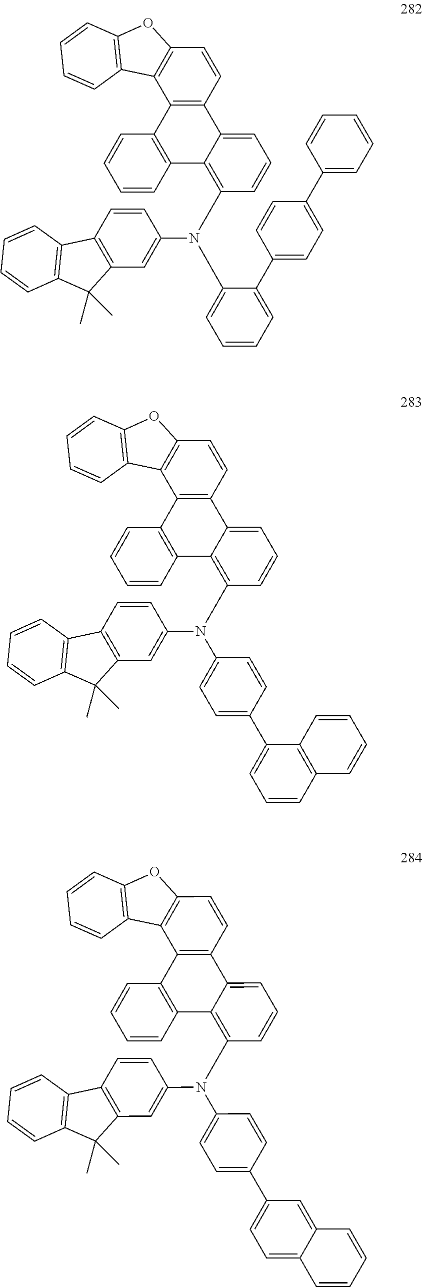 [SMILES: CC1(C)c2ccccc2-c2ccc(N(c3ccccc3-c3ccc(-c4ccccc4)cc3)c3cccc4c5ccc6oc7ccccc7c6c5c5ccccc5c34)cc21 ; CC1(C)c2ccccc2-c2ccc(N(c3ccc(-c4cccc5ccccc45)cc3)c3cccc4c5ccc6oc7ccccc7c6c5c5ccccc5c34)cc21 ; CC1(C)c2ccccc2-c2ccc(N(c3ccc(-c4ccc5ccccc5c4)cc3)c3cccc4c5ccc6oc7ccccc7c6c5c5ccccc5c34)cc21]

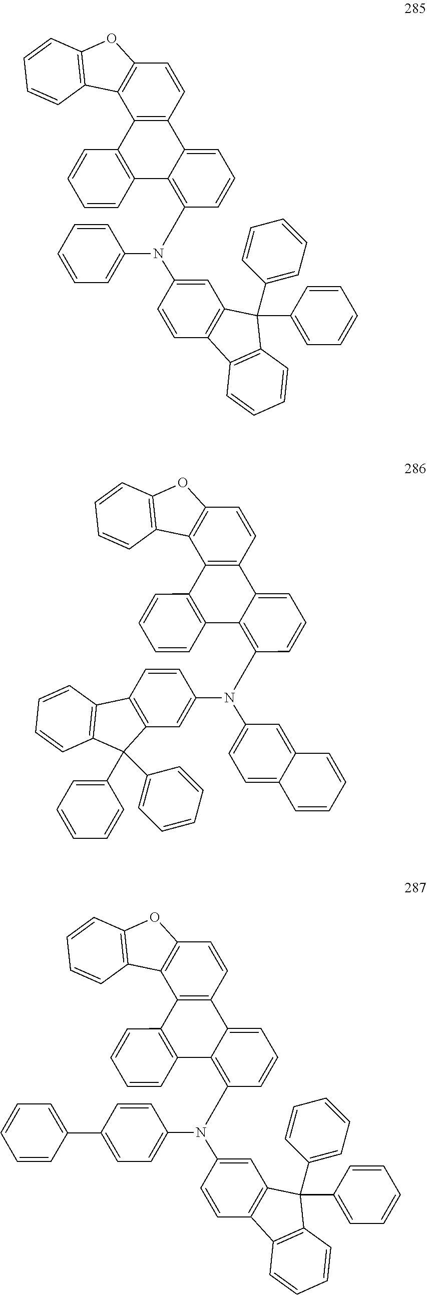 [SMILES: c1ccc(N(c2ccc3c(c2)C(c2ccccc2)(c2ccccc2)c2ccccc2-3)c2cccc3c4ccc5oc6ccccc6c5c4c4ccccc4c23)cc1 ; c1ccc(C2(c3ccccc3)c3ccccc3-c3ccc(N(c4ccc5ccccc5c4)c4cccc5c6ccc7oc8ccccc8c7c6c6ccccc6c45)cc32)cc1 ; c1ccc(-c2ccc(N(c3ccc4c(c3)C(c3ccccc3)(c3ccccc3)c3ccccc3-4)c3cccc4c5ccc6oc7ccccc7c6c5c5ccccc5c34)cc2)cc1]

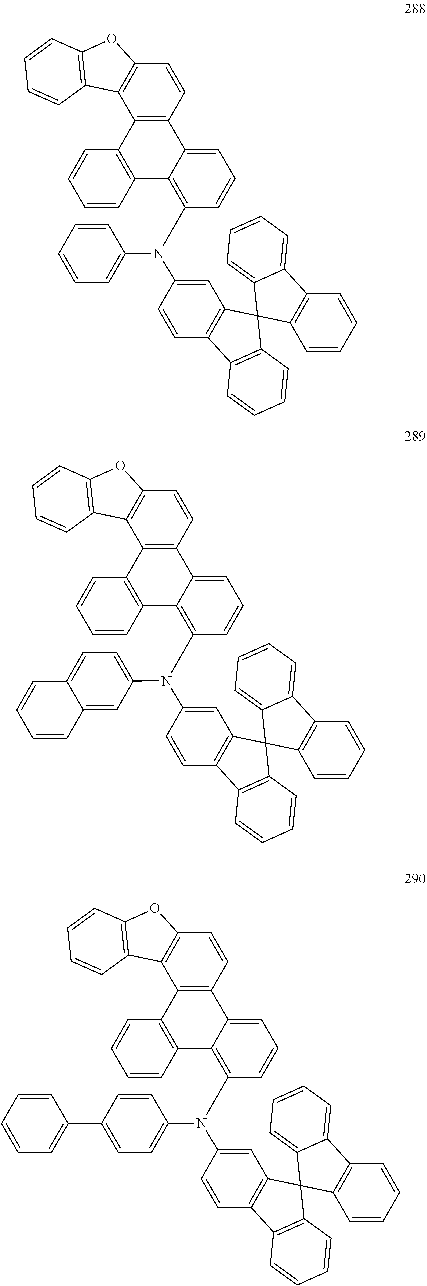 [SMILES: c1ccc(N(c2ccc3c(c2)C2(c4ccccc4-c4ccccc42)c2ccccc2-3)c2cccc3c4ccc5oc6ccccc6c5c4c4ccccc4c23)cc1 ; c1ccc2c(c1)-c1ccccc1C21c2ccccc2-c2ccc(N(c3ccc4ccccc4c3)c3cccc4c5ccc6oc7ccccc7c6c5c5ccccc5c34)cc21 ; c1ccc(-c2ccc(N(c3ccc4c(c3)C3(c5ccccc5-c5ccccc53)c3ccccc3-4)c3cccc4c5ccc6oc7ccccc7c6c5c5ccccc5c34)cc2)cc1]

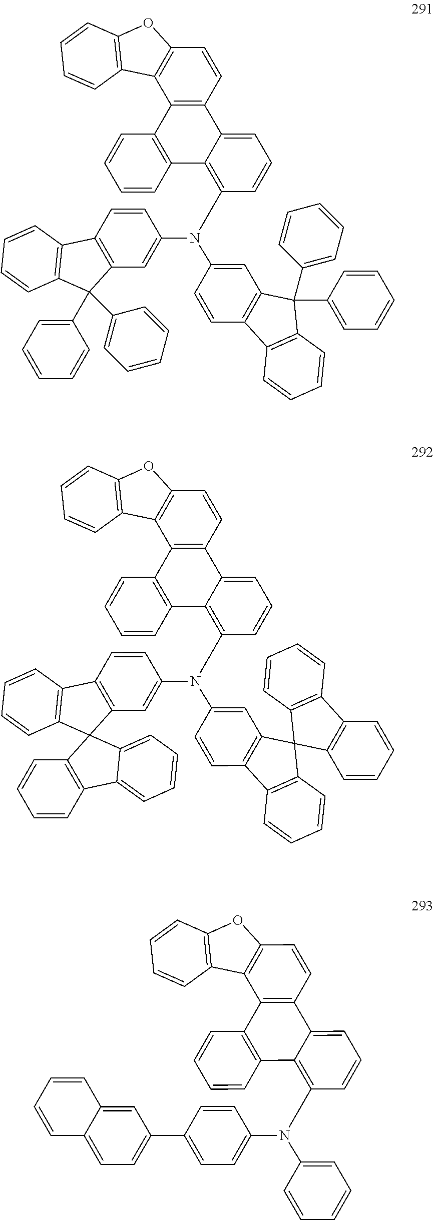 [SMILES: c1ccc(C2(c3ccccc3)c3ccccc3-c3ccc(N(c4ccc5c(c4)C(c4ccccc4)(c4ccccc4)c4ccccc4-5)c4cccc5c6ccc7oc8ccccc8c7c6c6ccccc6c45)cc32)cc1 ; c1ccc2c(c1)-c1ccccc1C21c2ccccc2-c2ccc(N(c3ccc4c(c3)C3(c5ccccc5-c5ccccc53)c3ccccc3-4)c3cccc4c5ccc6oc7ccccc7c6c5c5ccccc5c34)cc21 ; c1ccc(N(c2ccc(-c3ccc4ccccc4c3)cc2)c2cccc3c4ccc5oc6ccccc6c5c4c4ccccc4c23)cc1]

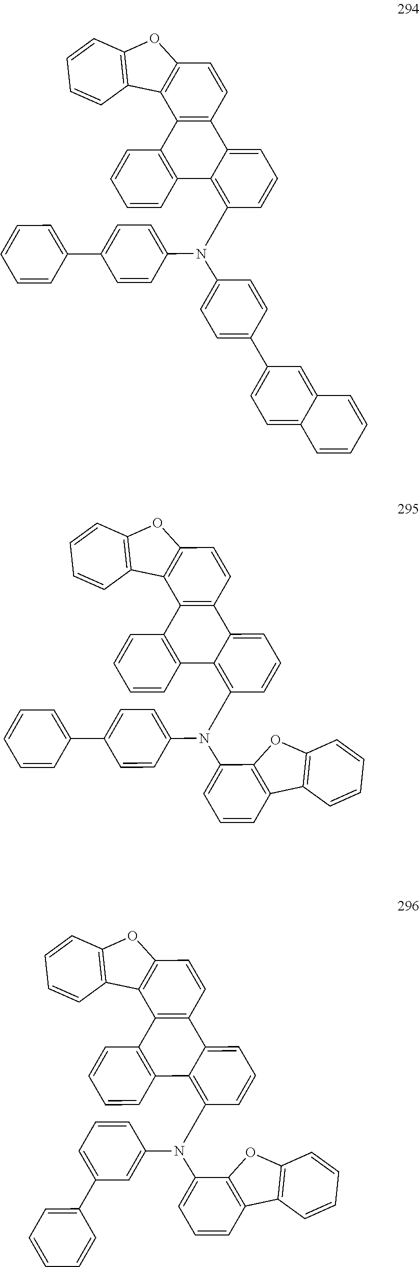 [SMILES: c1ccc(-c2ccc(N(c3ccc(-c4ccc5ccccc5c4)cc3)c3cccc4c5ccc6oc7ccccc7c6c5c5ccccc5c34)cc2)cc1 ; c1ccc(-c2ccc(N(c3cccc4c3oc3ccccc34)c3cccc4c5ccc6oc7ccccc7c6c5c5ccccc5c34)cc2)cc1 ; c1ccc(-c2cccc(N(c3cccc4c3oc3ccccc34)c3cccc4c5ccc6oc7ccccc7c6c5c5ccccc5c34)c2)cc1]

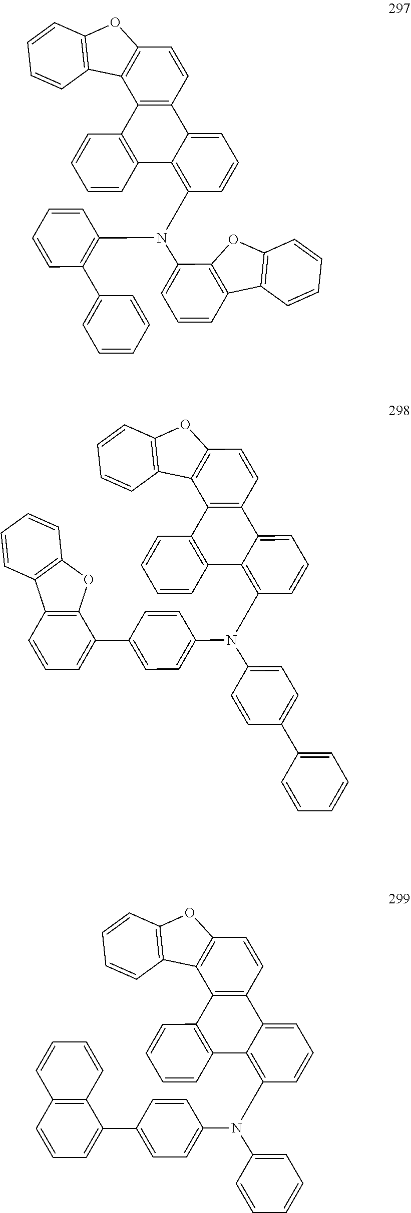 [SMILES: c1ccc(-c2ccccc2N(c2cccc3c2oc2ccccc23)c2cccc3c4ccc5oc6ccccc6c5c4c4ccccc4c23)cc1 ; c1ccc(-c2ccc(N(c3ccc(-c4cccc5c4oc4ccccc45)cc3)c3cccc4c5ccc6oc7ccccc7c6c5c5ccccc5c34)cc2)cc1 ; c1ccc(N(c2ccc(-c3cccc4ccccc34)cc2)c2cccc3c4ccc5oc6ccccc6c5c4c4ccccc4c23)cc1]

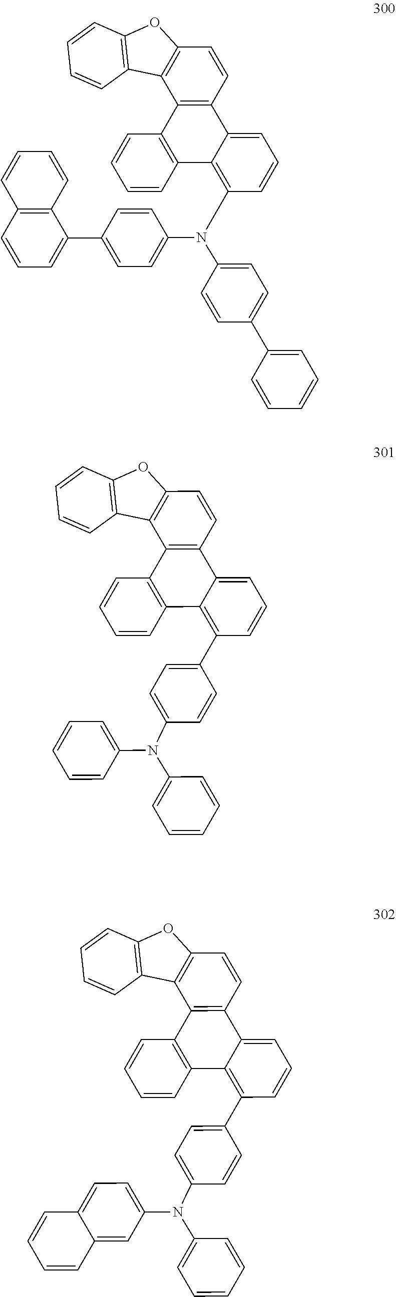 [SMILES: c1ccc(-c2ccc(N(c3ccc(-c4cccc5ccccc45)cc3)c3cccc4c5ccc6oc7ccccc7c6c5c5ccccc5c34)cc2)cc1 ; c1ccc(N(c2ccccc2)c2ccc(-c3cccc4c5ccc6oc7ccccc7c6c5c5ccccc5c34)cc2)cc1 ; c1ccc(N(c2ccc(-c3cccc4c5ccc6oc7ccccc7c6c5c5ccccc5c34)cc2)c2ccc3ccccc3c2)cc1]

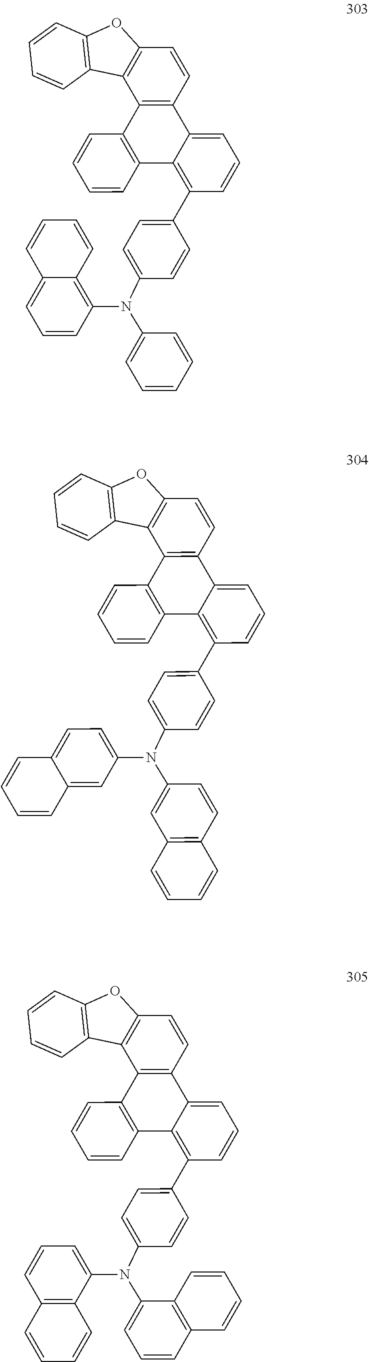 [SMILES: c1ccc(N(c2ccc(-c3cccc4c5ccc6oc7ccccc7c6c5c5ccccc5c34)cc2)c2cccc3ccccc23)cc1 ; c1ccc2cc(N(c3ccc(-c4cccc5c6ccc7oc8ccccc8c7c6c6ccccc6c45)cc3)c3ccc4ccccc4c3)ccc2c1 ; c1ccc2c(N(c3ccc(-c4cccc5c6ccc7oc8ccccc8c7c6c6ccccc6c45)cc3)c3cccc4ccccc34)cccc2c1]

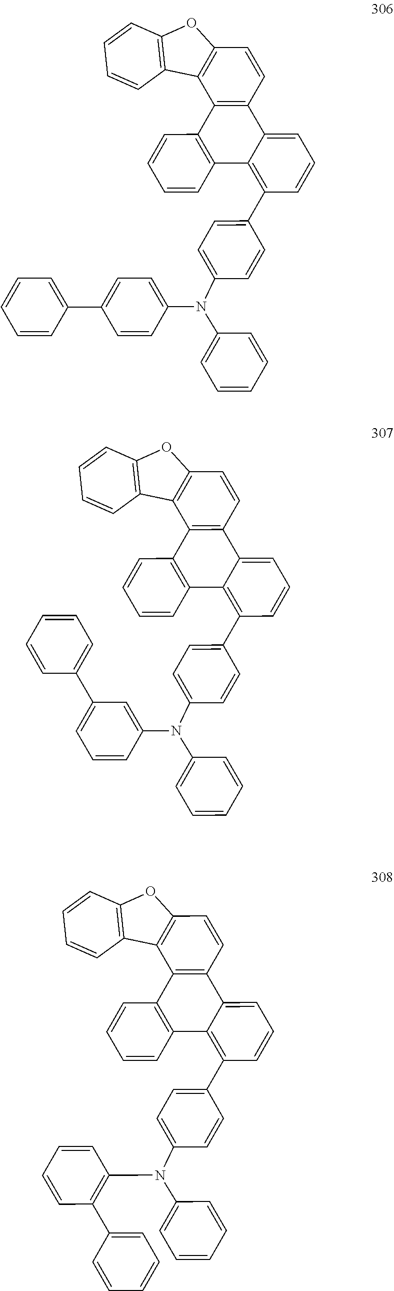 [SMILES: c1ccc(-c2ccc(N(c3ccccc3)c3ccc(-c4cccc5c6ccc7oc8ccccc8c7c6c6ccccc6c45)cc3)cc2)cc1 ; c1ccc(-c2cccc(N(c3ccccc3)c3ccc(-c4cccc5c6ccc7oc8ccccc8c7c6c6ccccc6c45)cc3)c2)cc1 ; c1ccc(-c2ccccc2N(c2ccccc2)c2ccc(-c3cccc4c5ccc6oc7ccccc7c6c5c5ccccc5c34)cc2)cc1]

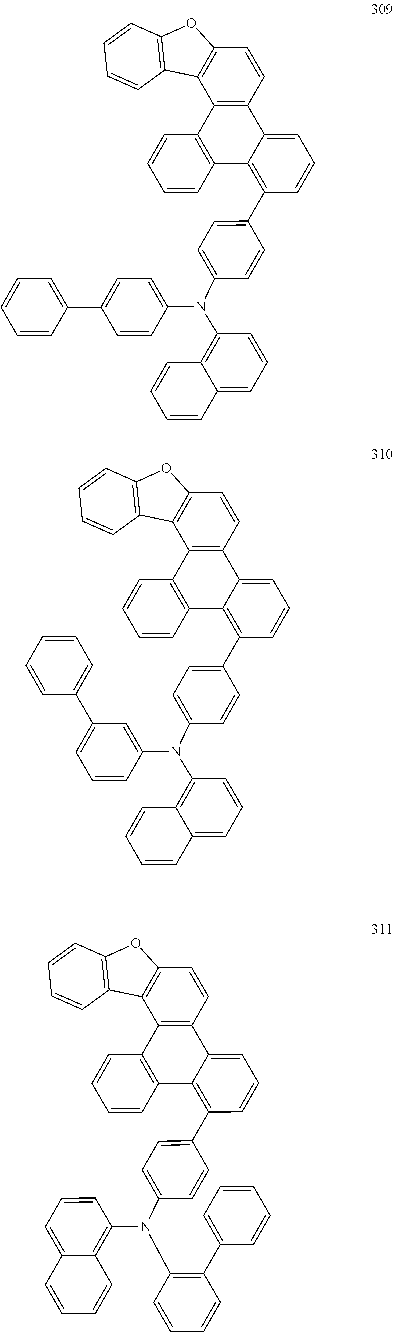 [SMILES: c1ccc(-c2ccc(N(c3ccc(-c4cccc5c6ccc7oc8ccccc8c7c6c6ccccc6c45)cc3)c3cccc4ccccc34)cc2)cc1 ; c1ccc(-c2cccc(N(c3ccc(-c4cccc5c6ccc7oc8ccccc8c7c6c6ccccc6c45)cc3)c3cccc4ccccc34)c2)cc1 ; c1ccc(-c2ccccc2N(c2ccc(-c3cccc4c5ccc6oc7ccccc7c6c5c5ccccc5c34)cc2)c2cccc3ccccc23)cc1]

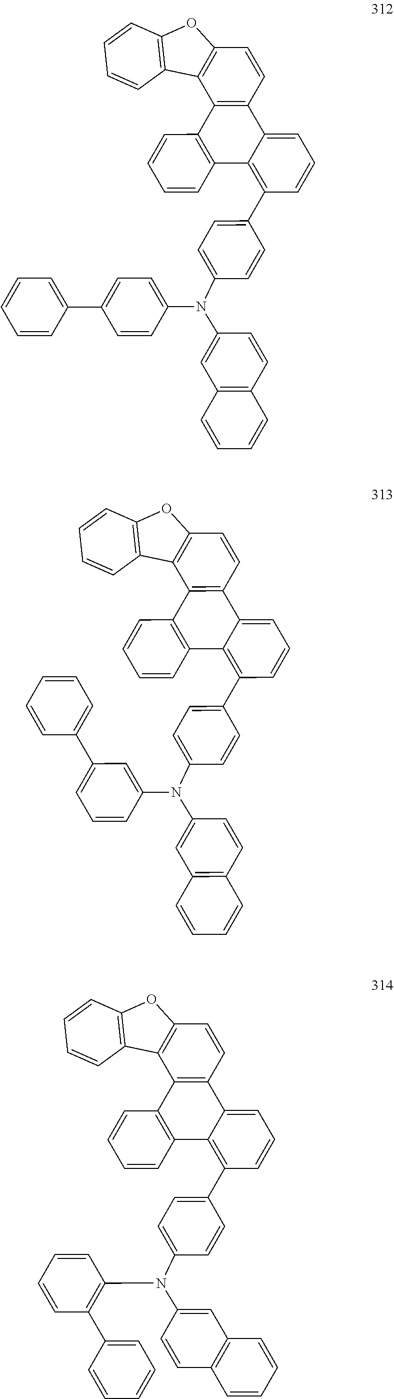 [SMILES: c1ccc(-c2ccc(N(c3ccc(-c4cccc5c6ccc7oc8ccccc8c7c6c6ccccc6c45)cc3)c3ccc4ccccc4c3)cc2)cc1 ; c1ccc(-c2cccc(N(c3ccc(-c4cccc5c6ccc7oc8ccccc8c7c6c6ccccc6c45)cc3)c3ccc4ccccc4c3)c2)cc1 ; c1ccc(-c2ccccc2N(c2ccc(-c3cccc4c5ccc6oc7ccccc7c6c5c5ccccc5c34)cc2)c2ccc3ccccc3c2)cc1]

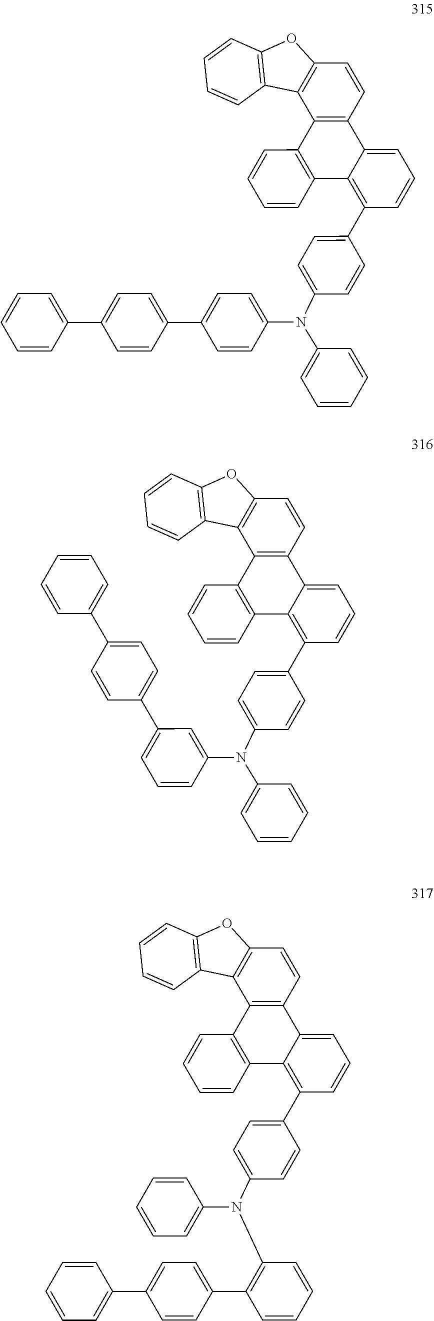 [SMILES: c1ccc(-c2ccc(-c3ccc(N(c4ccccc4)c4ccc(-c5cccc6c7ccc8oc9ccccc9c8c7c7ccccc7c56)cc4)cc3)cc2)cc1 ; c1ccc(-c2ccc(-c3cccc(N(c4ccccc4)c4ccc(-c5cccc6c7ccc8oc9ccccc9c8c7c7ccccc7c56)cc4)c3)cc2)cc1 ; c1ccc(-c2ccc(-c3ccccc3N(c3ccccc3)c3ccc(-c4cccc5c6ccc7oc8ccccc8c7c6c6ccccc6c45)cc3)cc2)cc1]

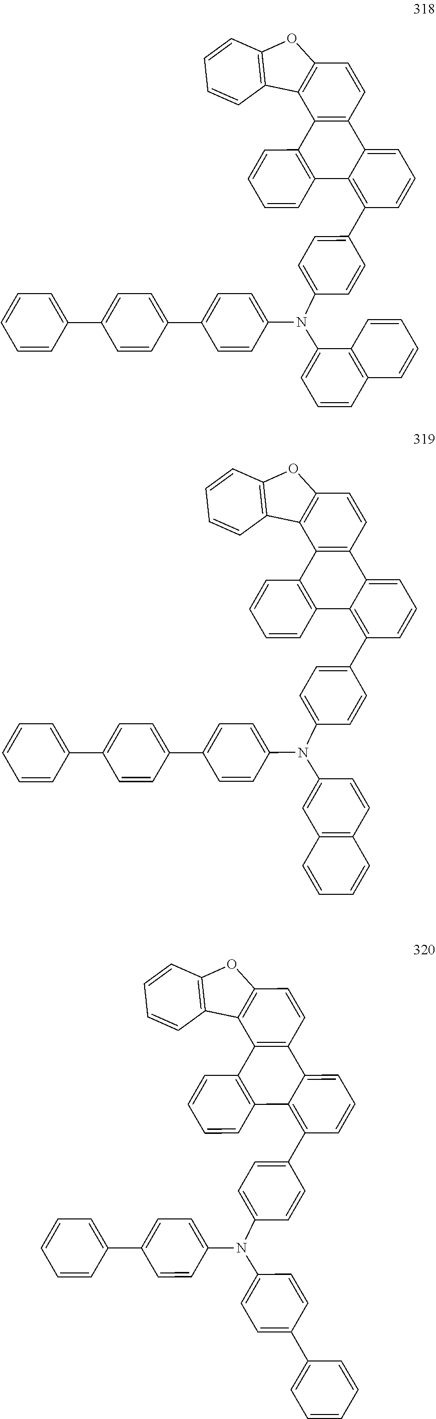 [SMILES: c1ccc(-c2ccc(-c3ccc(N(c4ccc(-c5cccc6c7ccc8oc9ccccc9c8c7c7ccccc7c56)cc4)c4cccc5ccccc45)cc3)cc2)cc1 ; c1ccc(-c2ccc(-c3ccc(N(c4ccc(-c5cccc6c7ccc8oc9ccccc9c8c7c7ccccc7c56)cc4)c4ccc5ccccc5c4)cc3)cc2)cc1 ; c1ccc(-c2ccc(N(c3ccc(-c4ccccc4)cc3)c3ccc(-c4cccc5c6ccc7oc8ccccc8c7c6c6ccccc6c45)cc3)cc2)cc1]

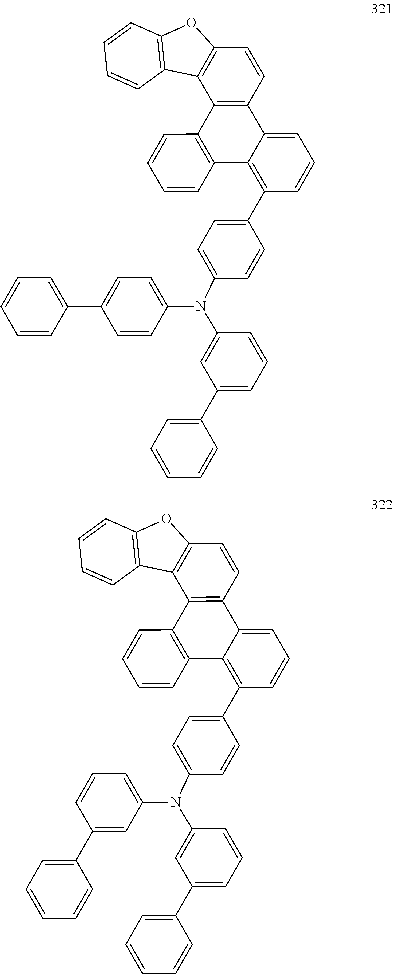

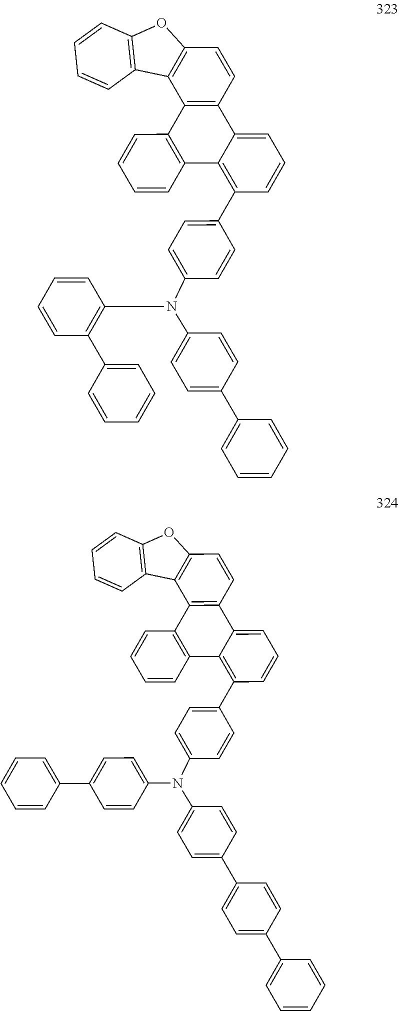 [SMILES: c1ccc(-c2ccc(N(c3ccc(-c4cccc5c6ccc7oc8ccccc8c7c6c6ccccc6c45)cc3)c3ccccc3-c3ccccc3)cc2)cc1 ; c1ccc(-c2ccc(-c3ccc(N(c4ccc(-c5ccccc5)cc4)c4ccc(-c5cccc6c7ccc8oc9ccccc9c8c7c7ccccc7c56)cc4)cc3)cc2)cc1]

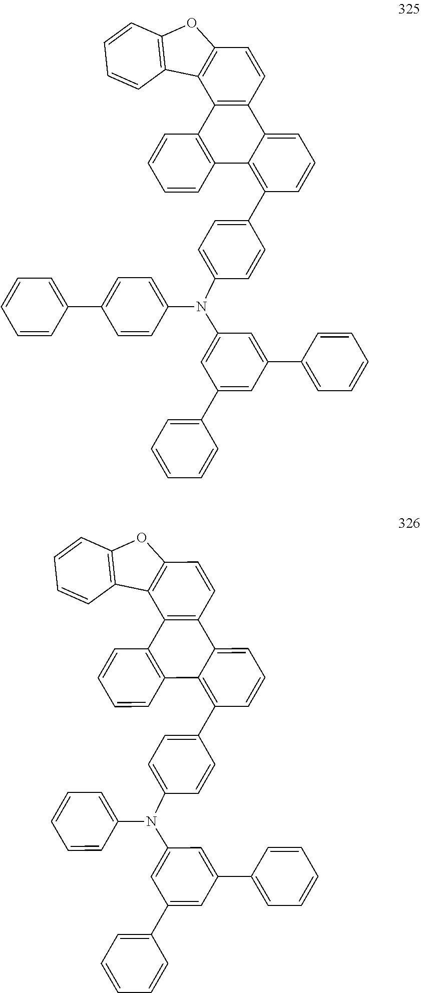 [SMILES: c1ccc(-c2ccc(N(c3ccc(-c4cccc5c6ccc7oc8ccccc8c7c6c6ccccc6c45)cc3)c3cc(-c4ccccc4)cc(-c4ccccc4)c3)cc2)cc1 ; c1ccc(-c2cc(-c3ccccc3)cc(N(c3ccccc3)c3ccc(-c4cccc5c6ccc7oc8ccccc8c7c6c6ccccc6c45)cc3)c2)cc1]

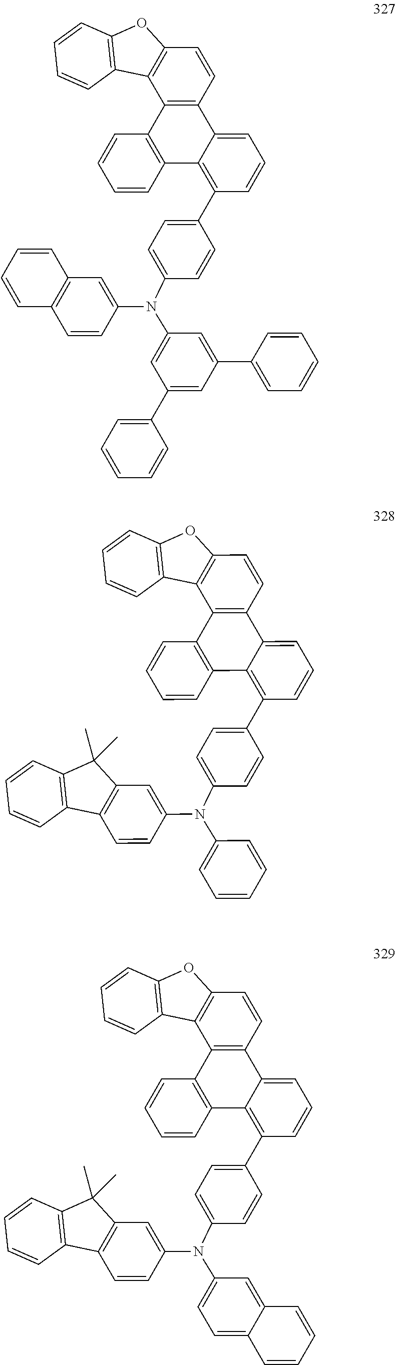 [SMILES: c1ccc(-c2cc(-c3ccccc3)cc(N(c3ccc(-c4cccc5c6ccc7oc8ccccc8c7c6c6ccccc6c45)cc3)c3ccc4ccccc4c3)c2)cc1 ; CC1(C)c2ccccc2-c2ccc(N(c3ccccc3)c3ccc(-c4cccc5c6ccc7oc8ccccc8c7c6c6ccccc6c45)cc3)cc21 ; CC1(C)c2ccccc2-c2ccc(N(c3ccc(-c4cccc5c6ccc7oc8ccccc8c7c6c6ccccc6c45)cc3)c3ccc4ccccc4c3)cc21]

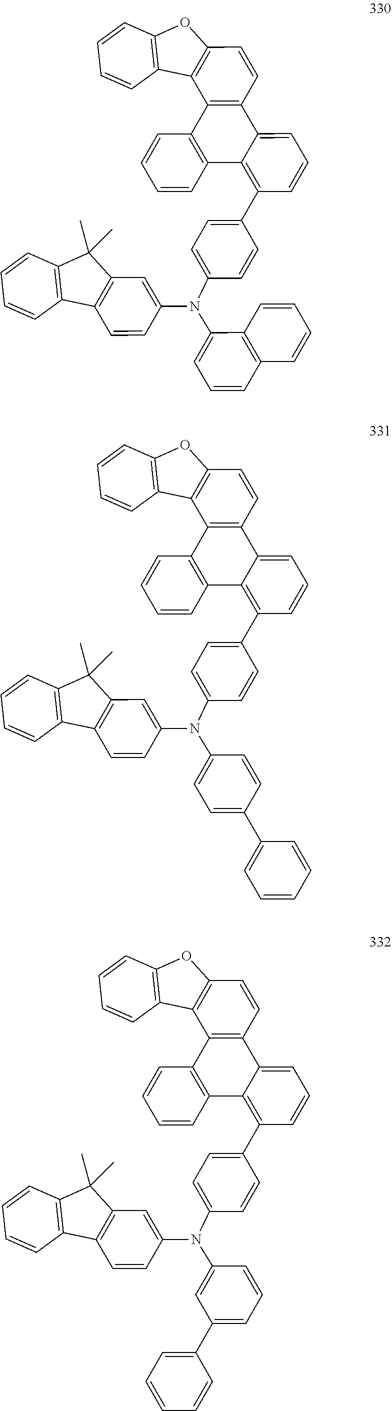 [SMILES: CC1(C)c2ccccc2-c2ccc(N(c3ccc(-c4cccc5c6ccc7oc8ccccc8c7c6c6ccccc6c45)cc3)c3cccc4ccccc34)cc21 ; CC1(C)c2ccccc2-c2ccc(N(c3ccc(-c4ccccc4)cc3)c3ccc(-c4cccc5c6ccc7oc8ccccc8c7c6c6ccccc6c45)cc3)cc21 ; CC1(C)c2ccccc2-c2ccc(N(c3ccc(-c4cccc5c6ccc7oc8ccccc8c7c6c6ccccc6c45)cc3)c3cccc(-c4ccccc4)c3)cc21]

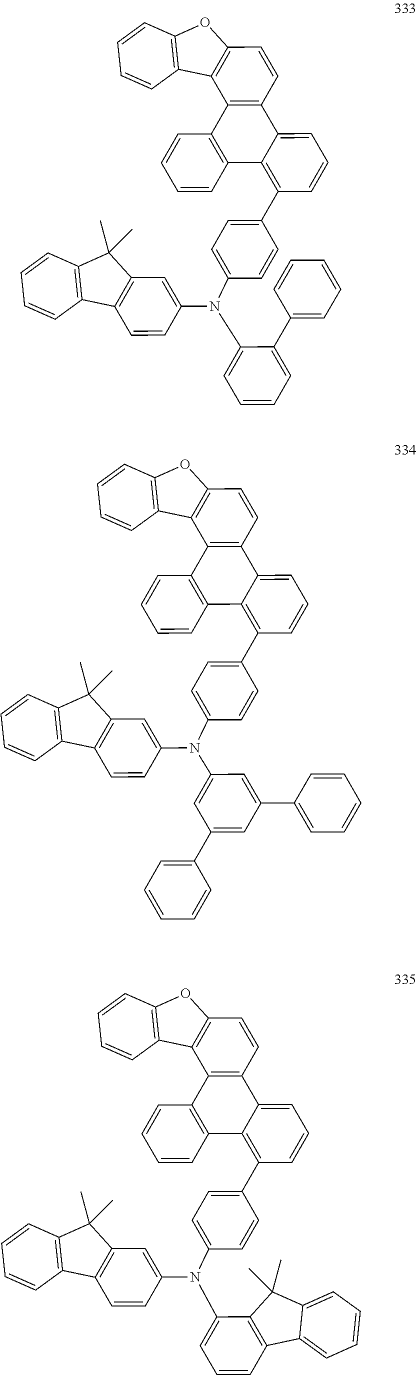 [SMILES: CC1(C)c2ccccc2-c2ccc(N(c3ccc(-c4cccc5c6ccc7oc8ccccc8c7c6c6ccccc6c45)cc3)c3ccccc3-c3ccccc3)cc21 ; CC1(C)c2ccccc2-c2ccc(N(c3ccc(-c4cccc5c6ccc7oc8ccccc8c7c6c6ccccc6c45)cc3)c3cc(-c4ccccc4)cc(-c4ccccc4)c3)cc21 ; CC1(C)c2ccccc2-c2ccc(N(c3ccc(-c4cccc5c6ccc7oc8ccccc8c7c6c6ccccc6c45)cc3)c3cccc4c3C(C)(C)c3ccccc3-4)cc21]

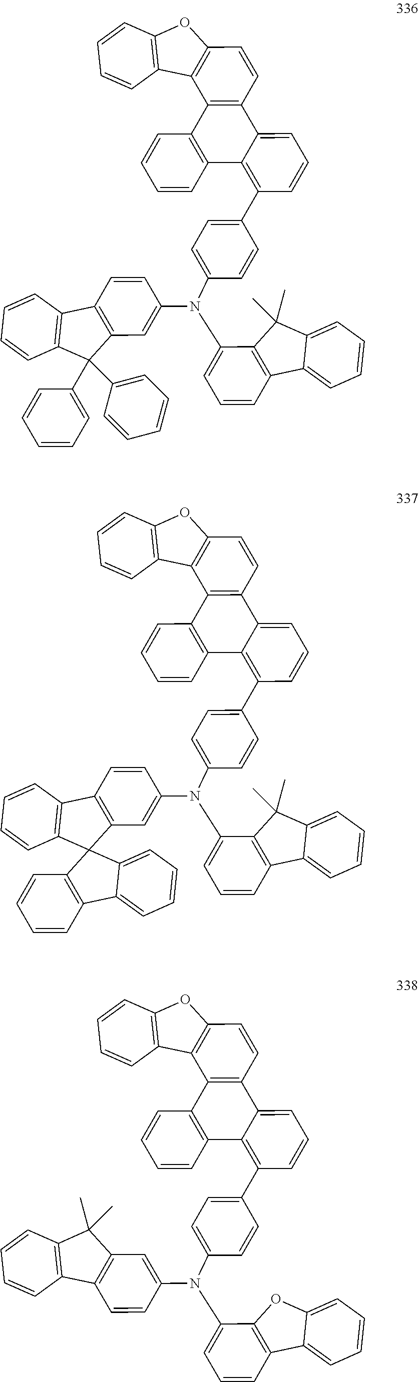 [SMILES: CC1(C)c2ccccc2-c2cccc(N(c3ccc(-c4cccc5c6ccc7oc8ccccc8c7c6c6ccccc6c45)cc3)c3ccc4c(c3)C(c3ccccc3)(c3ccccc3)c3ccccc3-4)c21 ; CC1(C)c2ccccc2-c2cccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)c21 ; CC1(C)c2ccccc2-c2ccc(N(c3ccc(-c4cccc5c6ccc7oc8ccccc8c7c6c6ccccc6c45)cc3)c3cccc4c3oc3ccccc34)cc21]

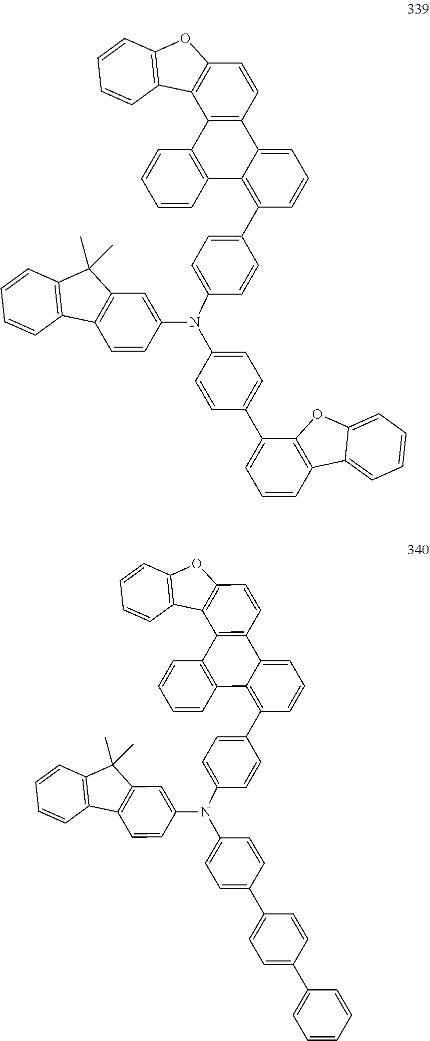 [SMILES: CC1(C)c2ccccc2-c2ccc(N(c3ccc(-c4cccc5c4oc4ccccc45)cc3)c3ccc(-c4cccc5c6ccc7oc8ccccc8c7c6c6ccccc6c45)cc3)cc21 ; CC1(C)c2ccccc2-c2ccc(N(c3ccc(-c4ccc(-c5ccccc5)cc4)cc3)c3ccc(-c4cccc5c6ccc7oc8ccccc8c7c6c6ccccc6c45)cc3)cc21]

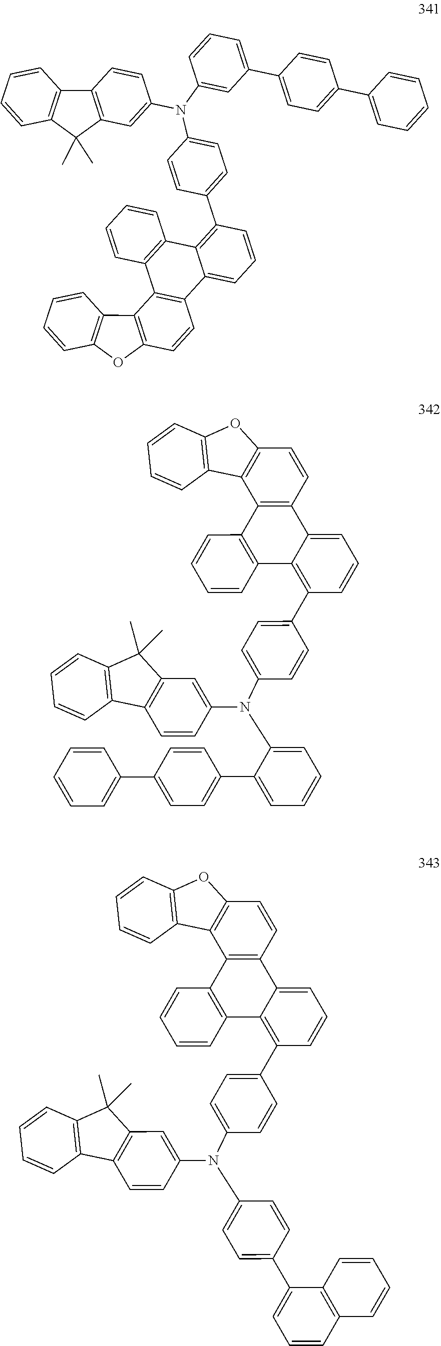 [SMILES: CC1(C)c2ccccc2-c2ccc(N(c3ccc(-c4cccc5c6ccc7oc8ccccc8c7c6c6ccccc6c45)cc3)c3cccc(-c4ccc(-c5ccccc5)cc4)c3)cc21 ; CC1(C)c2ccccc2-c2ccc(N(c3ccc(-c4cccc5c6ccc7oc8ccccc8c7c6c6ccccc6c45)cc3)c3ccccc3-c3ccc(-c4ccccc4)cc3)cc21 ; CC1(C)c2ccccc2-c2ccc(N(c3ccc(-c4cccc5ccccc45)cc3)c3ccc(-c4cccc5c6ccc7oc8ccccc8c7c6c6ccccc6c45)cc3)cc21]

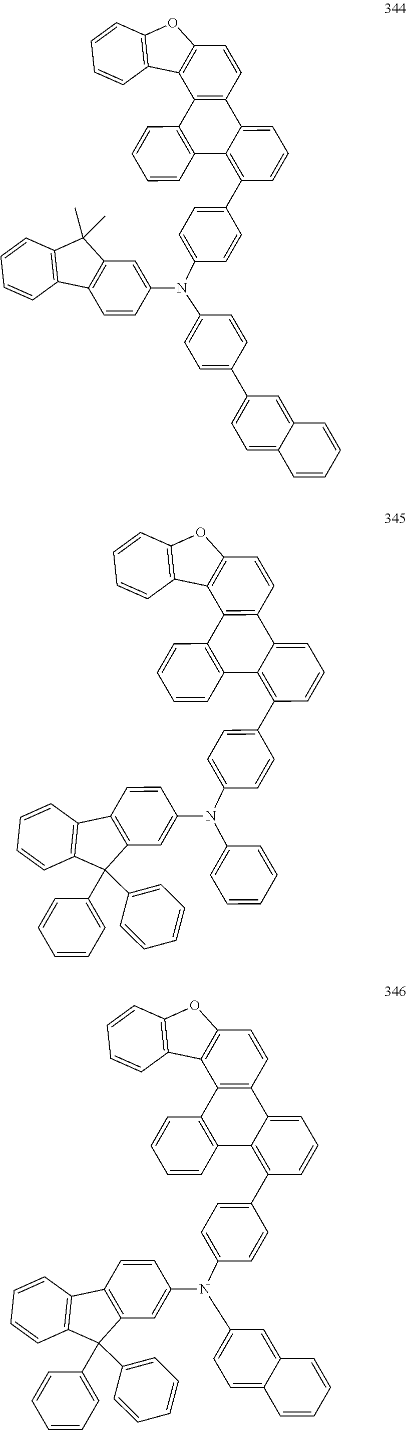 [SMILES: CC1(C)c2ccccc2-c2ccc(N(c3ccc(-c4ccc5ccccc5c4)cc3)c3ccc(-c4cccc5c6ccc7oc8ccccc8c7c6c6ccccc6c45)cc3)cc21 ; c1ccc(N(c2ccc(-c3cccc4c5ccc6oc7ccccc7c6c5c5ccccc5c34)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cccc6c7ccc8oc9ccccc9c8c7c7ccccc7c56)cc4)c4ccc5ccccc5c4)cc32)cc1]

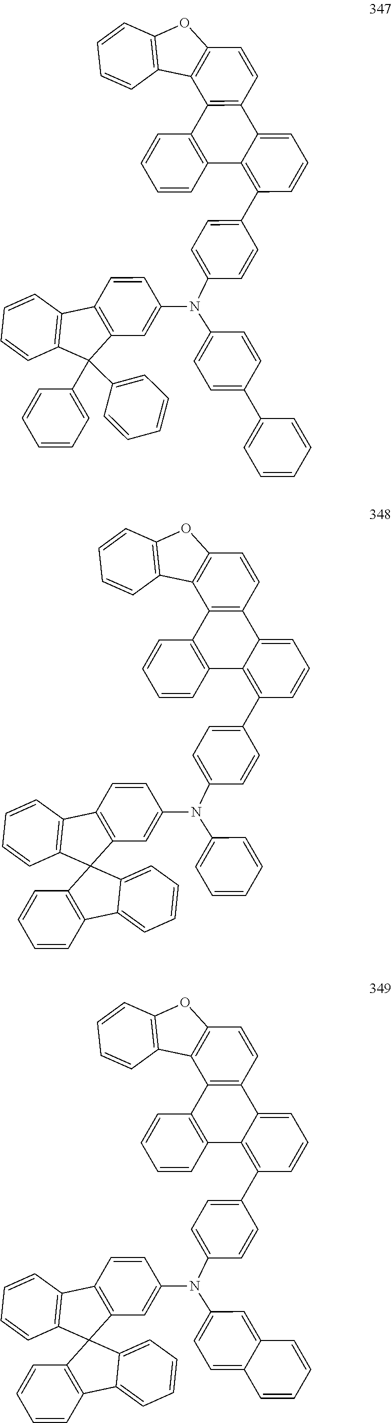 [SMILES: c1ccc(-c2ccc(N(c3ccc(-c4cccc5c6ccc7oc8ccccc8c7c6c6ccccc6c45)cc3)c3ccc4c(c3)C(c3ccccc3)(c3ccccc3)c3ccccc3-4)cc2)cc1 ; c1ccc(N(c2ccc(-c3cccc4c5ccc6oc7ccccc7c6c5c5ccccc5c34)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4cccc5c6ccc7oc8ccccc8c7c6c6ccccc6c45)cc3)c3ccc4ccccc4c3)cc21]

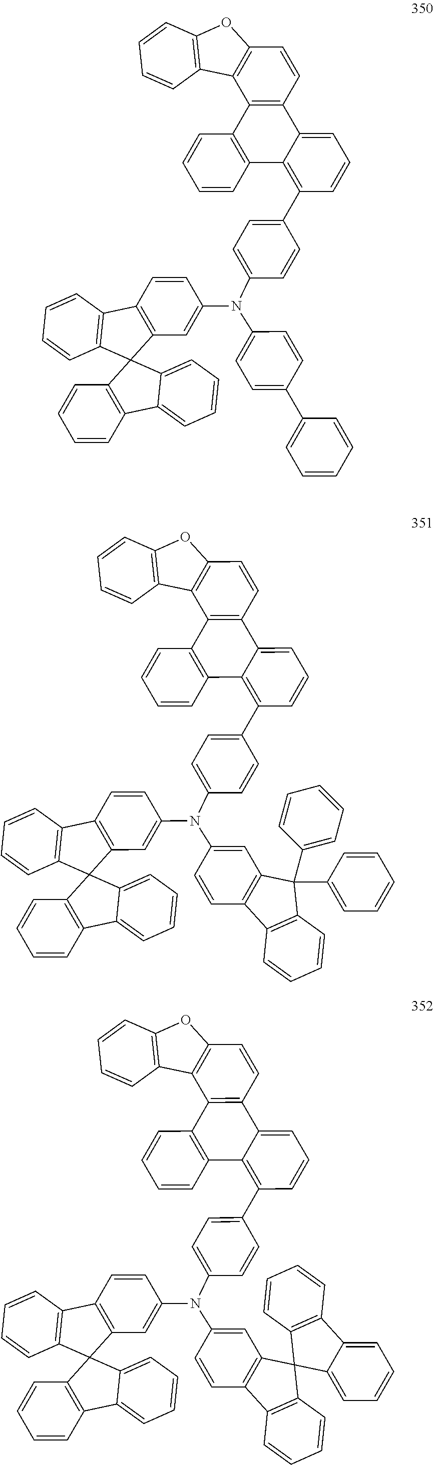 [SMILES: c1ccc(-c2ccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)cc2)cc1 ; c1ccc(C2(c3ccccc3)c3ccccc3-c3ccc(N(c4ccc(-c5cccc6c7ccc8oc9ccccc9c8c7c7ccccc7c56)cc4)c4ccc5c(c4)C4(c6ccccc6-c6ccccc64)c4ccccc4-5)cc32)cc1 ; c1ccc2c(c1)-c1ccccc1C21c2ccccc2-c2ccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)cc21]

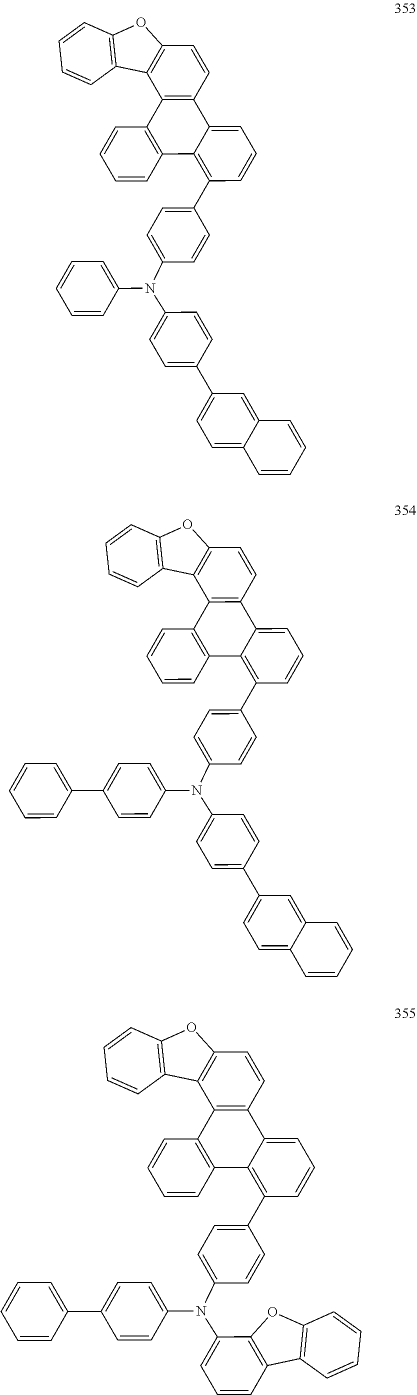 [SMILES: c1ccc(N(c2ccc(-c3ccc4ccccc4c3)cc2)c2ccc(-c3cccc4c5ccc6oc7ccccc7c6c5c5ccccc5c34)cc2)cc1 ; c1ccc(-c2ccc(N(c3ccc(-c4ccc5ccccc5c4)cc3)c3ccc(-c4cccc5c6ccc7oc8ccccc8c7c6c6ccccc6c45)cc3)cc2)cc1 ; c1ccc(-c2ccc(N(c3ccc(-c4cccc5c6ccc7oc8ccccc8c7c6c6ccccc6c45)cc3)c3cccc4c3oc3ccccc34)cc2)cc1]

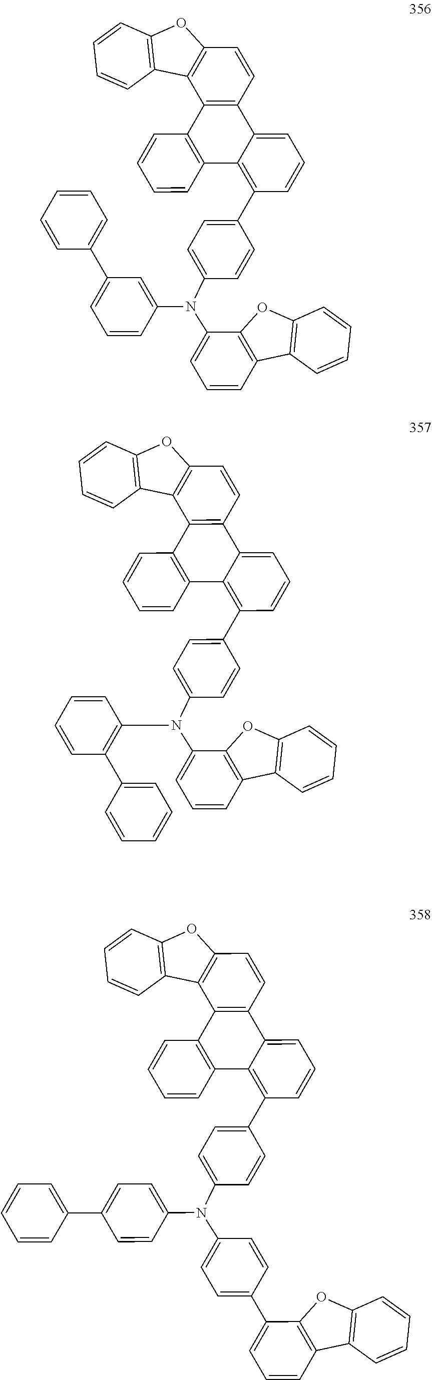 [SMILES: c1ccc(-c2cccc(N(c3ccc(-c4cccc5c6ccc7oc8ccccc8c7c6c6ccccc6c45)cc3)c3cccc4c3oc3ccccc34)c2)cc1 ; c1ccc(-c2ccccc2N(c2ccc(-c3cccc4c5ccc6oc7ccccc7c6c5c5ccccc5c34)cc2)c2cccc3c2oc2ccccc23)cc1 ; c1ccc(-c2ccc(N(c3ccc(-c4cccc5c4oc4ccccc45)cc3)c3ccc(-c4cccc5c6ccc7oc8ccccc8c7c6c6ccccc6c45)cc3)cc2)cc1]

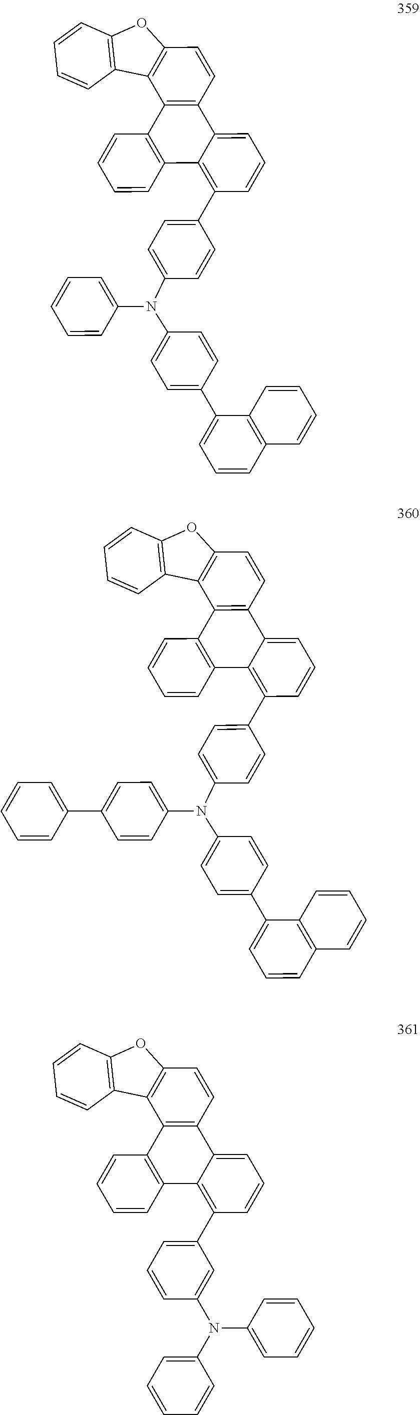 [SMILES: c1ccc(N(c2ccc(-c3cccc4ccccc34)cc2)c2ccc(-c3cccc4c5ccc6oc7ccccc7c6c5c5ccccc5c34)cc2)cc1 ; c1ccc(-c2ccc(N(c3ccc(-c4cccc5ccccc45)cc3)c3ccc(-c4cccc5c6ccc7oc8ccccc8c7c6c6ccccc6c45)cc3)cc2)cc1 ; c1ccc(N(c2ccccc2)c2cccc(-c3cccc4c5ccc6oc7ccccc7c6c5c5ccccc5c34)c2)cc1]

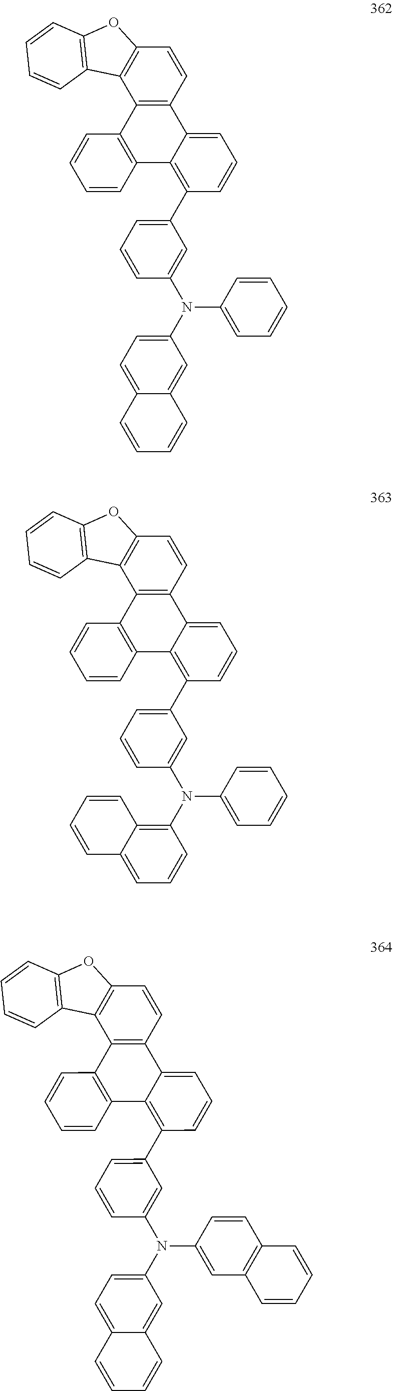 [SMILES: c1ccc(N(c2cccc(-c3cccc4c5ccc6oc7ccccc7c6c5c5ccccc5c34)c2)c2ccc3ccccc3c2)cc1 ; c1ccc(N(c2cccc(-c3cccc4c5ccc6oc7ccccc7c6c5c5ccccc5c34)c2)c2cccc3ccccc23)cc1 ; c1cc(-c2cccc3c4ccc5oc6ccccc6c5c4c4ccccc4c23)cc(N(c2ccc3ccccc3c2)c2ccc3ccccc3c2)c1]

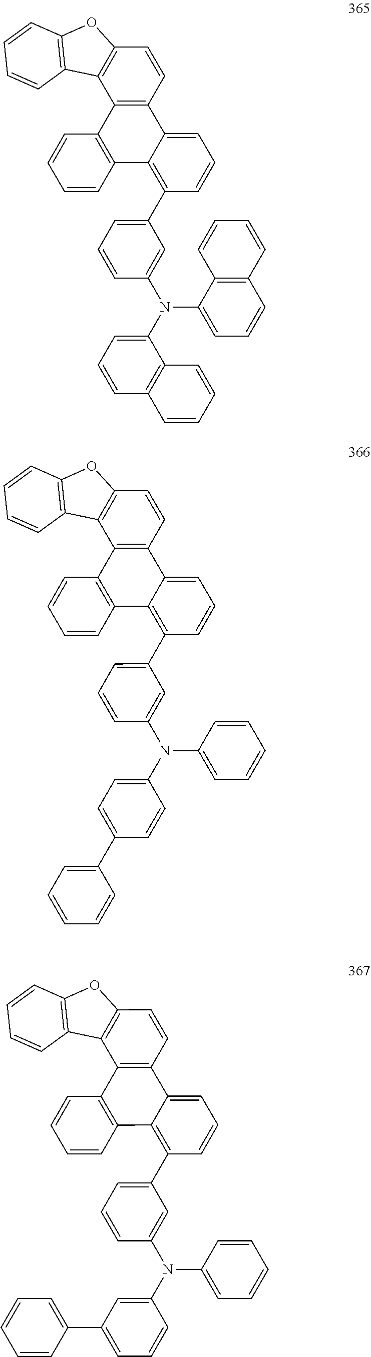 [SMILES: c1cc(-c2cccc3c4ccc5oc6ccccc6c5c4c4ccccc4c23)cc(N(c2cccc3ccccc23)c2cccc3ccccc23)c1 ; c1ccc(-c2ccc(N(c3ccccc3)c3cccc(-c4cccc5c6ccc7oc8ccccc8c7c6c6ccccc6c45)c3)cc2)cc1 ; c1ccc(-c2cccc(N(c3ccccc3)c3cccc(-c4cccc5c6ccc7oc8ccccc8c7c6c6ccccc6c45)c3)c2)cc1]

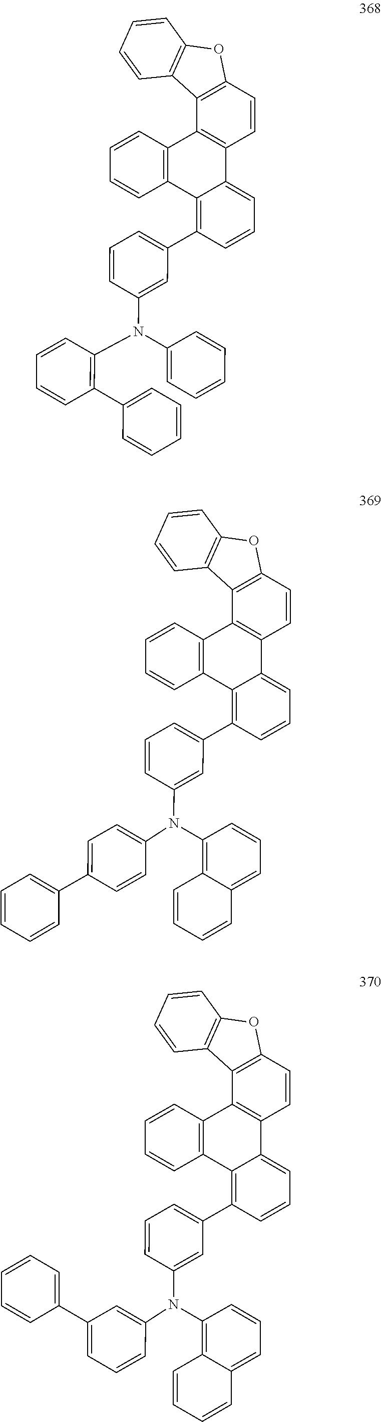 [SMILES: c1ccc(-c2ccccc2N(c2ccccc2)c2cccc(-c3cccc4c5ccc6oc7ccccc7c6c5c5ccccc5c34)c2)cc1 ; c1ccc(-c2ccc(N(c3cccc(-c4cccc5c6ccc7oc8ccccc8c7c6c6ccccc6c45)c3)c3cccc4ccccc34)cc2)cc1 ; c1ccc(-c2cccc(N(c3cccc(-c4cccc5c6ccc7oc8ccccc8c7c6c6ccccc6c45)c3)c3cccc4ccccc34)c2)cc1]

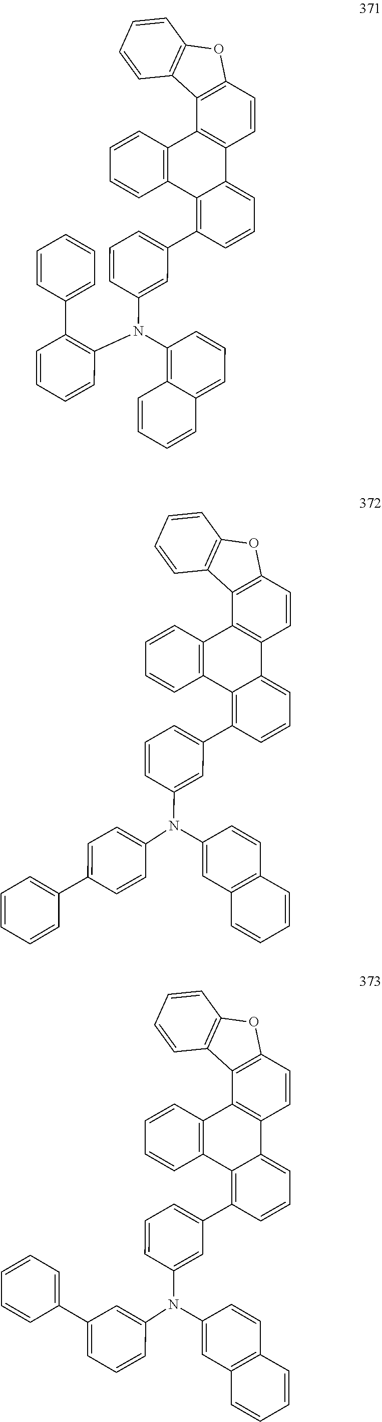 [SMILES: c1ccc(-c2ccccc2N(c2cccc(-c3cccc4c5ccc6oc7ccccc7c6c5c5ccccc5c34)c2)c2cccc3ccccc23)cc1 ; c1ccc(-c2ccc(N(c3cccc(-c4cccc5c6ccc7oc8ccccc8c7c6c6ccccc6c45)c3)c3ccc4ccccc4c3)cc2)cc1 ; c1ccc(-c2cccc(N(c3cccc(-c4cccc5c6ccc7oc8ccccc8c7c6c6ccccc6c45)c3)c3ccc4ccccc4c3)c2)cc1]

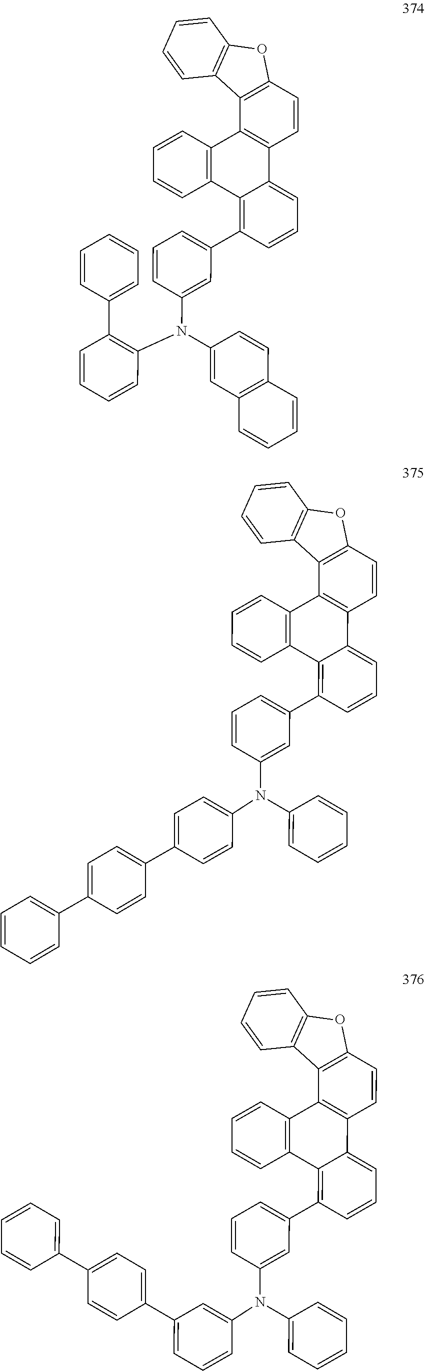

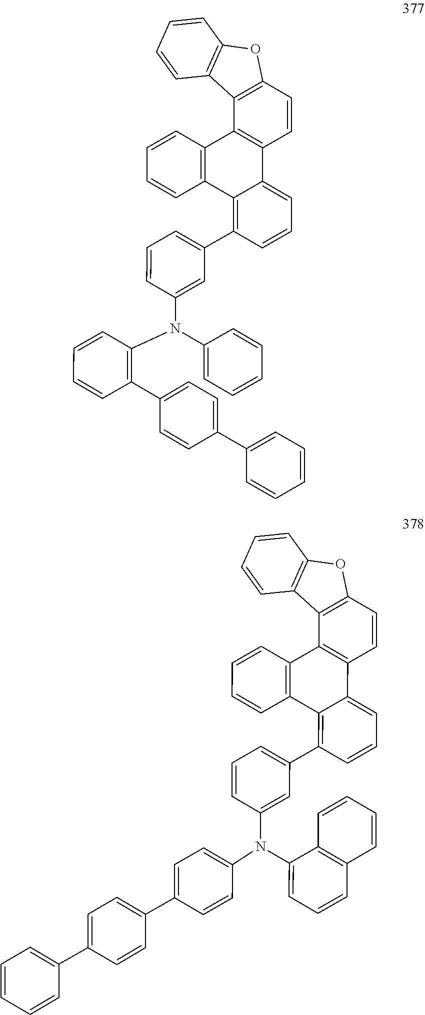

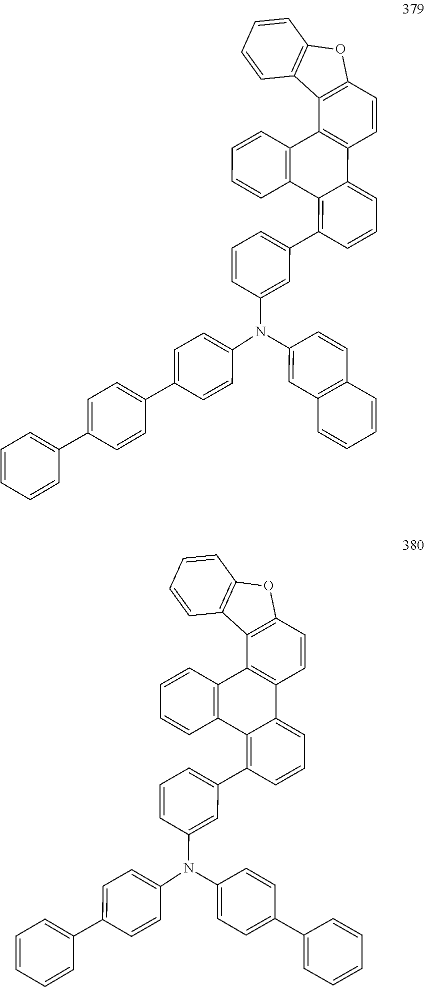 [SMILES: c1ccc(-c2ccc(-c3ccc(N(c4cccc(-c5cccc6c7ccc8oc9ccccc9c8c7c7ccccc7c56)c4)c4ccc5ccccc5c4)cc3)cc2)cc1 ; c1ccc(-c2ccc(N(c3ccc(-c4ccccc4)cc3)c3cccc(-c4cccc5c6ccc7oc8ccccc8c7c6c6ccccc6c45)c3)cc2)cc1]

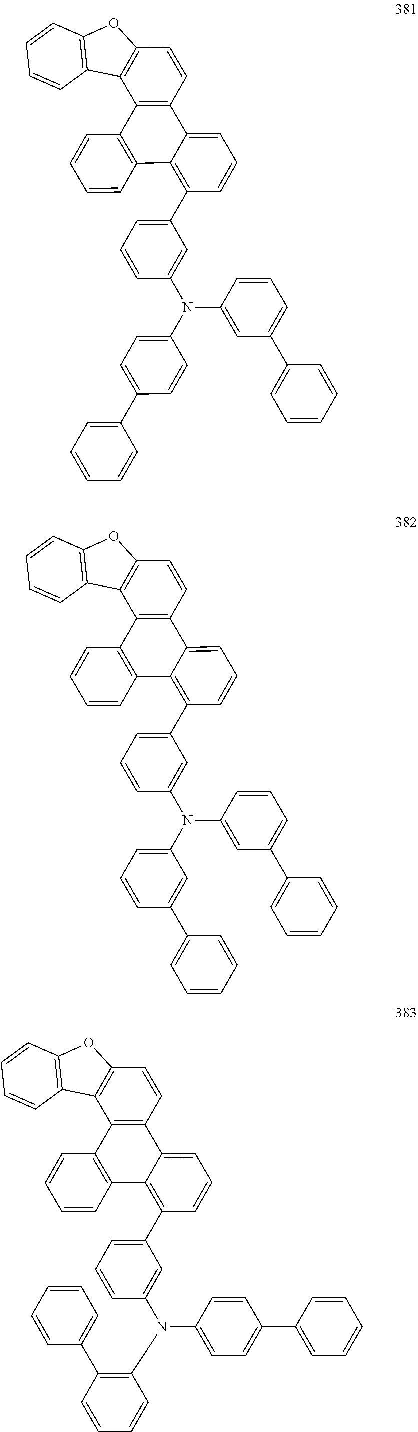 [SMILES: c1ccc(-c2ccc(N(c3cccc(-c4ccccc4)c3)c3cccc(-c4cccc5c6ccc7oc8ccccc8c7c6c6ccccc6c45)c3)cc2)cc1 ; c1ccc(-c2cccc(N(c3cccc(-c4ccccc4)c3)c3cccc(-c4cccc5c6ccc7oc8ccccc8c7c6c6ccccc6c45)c3)c2)cc1 ; c1ccc(-c2ccc(N(c3cccc(-c4cccc5c6ccc7oc8ccccc8c7c6c6ccccc6c45)c3)c3ccccc3-c3ccccc3)cc2)cc1]

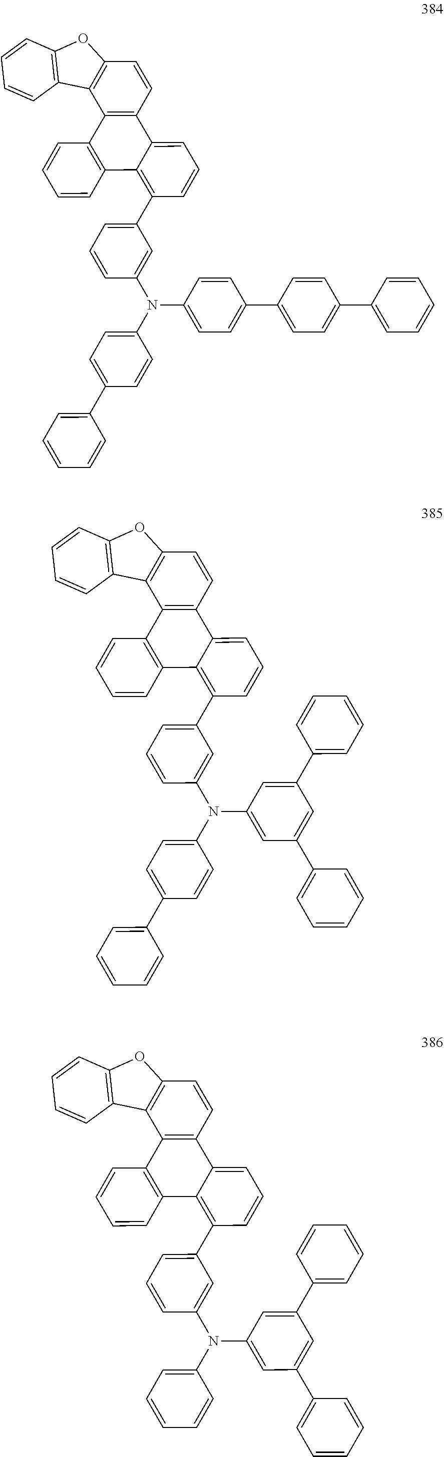 [SMILES: c1ccc(-c2ccc(-c3ccc(N(c4ccc(-c5ccccc5)cc4)c4cccc(-c5cccc6c7ccc8oc9ccccc9c8c7c7ccccc7c56)c4)cc3)cc2)cc1 ; c1ccc(-c2ccc(N(c3cc(-c4ccccc4)cc(-c4ccccc4)c3)c3cccc(-c4cccc5c6ccc7oc8ccccc8c7c6c6ccccc6c45)c3)cc2)cc1 ; c1ccc(-c2cc(-c3ccccc3)cc(N(c3ccccc3)c3cccc(-c4cccc5c6ccc7oc8ccccc8c7c6c6ccccc6c45)c3)c2)cc1]

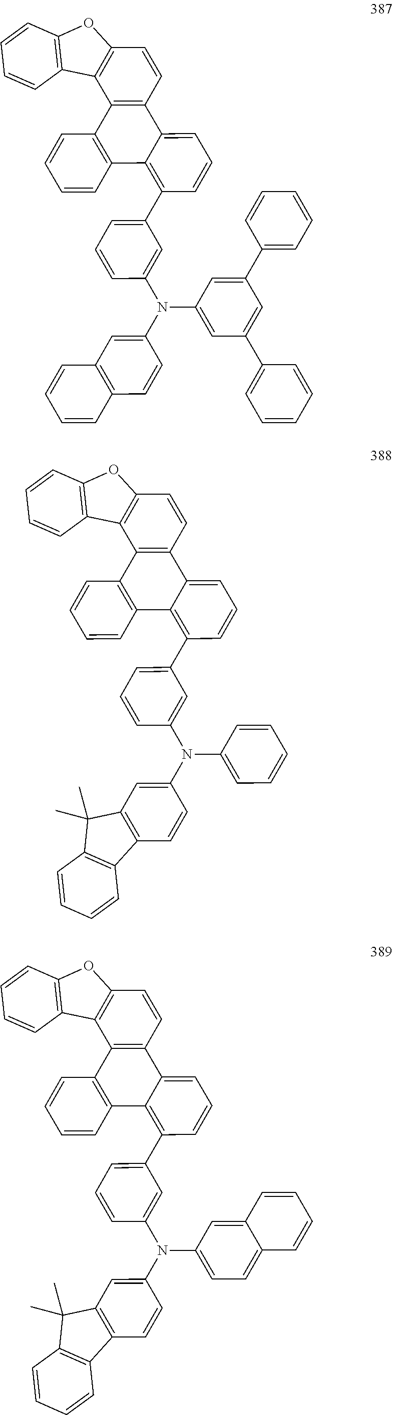 [SMILES: c1ccc(-c2cc(-c3ccccc3)cc(N(c3cccc(-c4cccc5c6ccc7oc8ccccc8c7c6c6ccccc6c45)c3)c3ccc4ccccc4c3)c2)cc1 ; CC1(C)c2ccccc2-c2ccc(N(c3ccccc3)c3cccc(-c4cccc5c6ccc7oc8ccccc8c7c6c6ccccc6c45)c3)cc21 ; CC1(C)c2ccccc2-c2ccc(N(c3cccc(-c4cccc5c6ccc7oc8ccccc8c7c6c6ccccc6c45)c3)c3ccc4ccccc4c3)cc21]

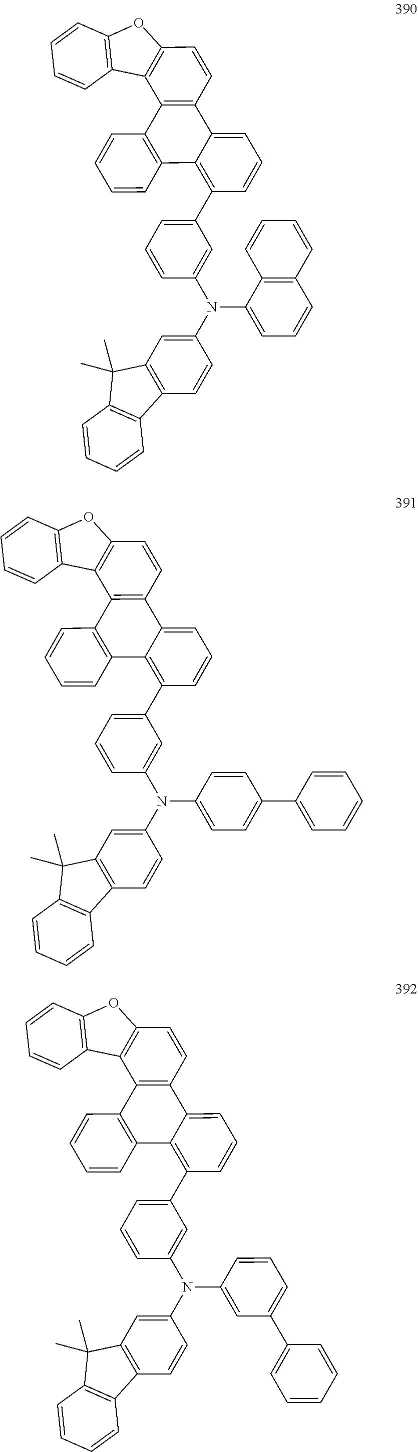

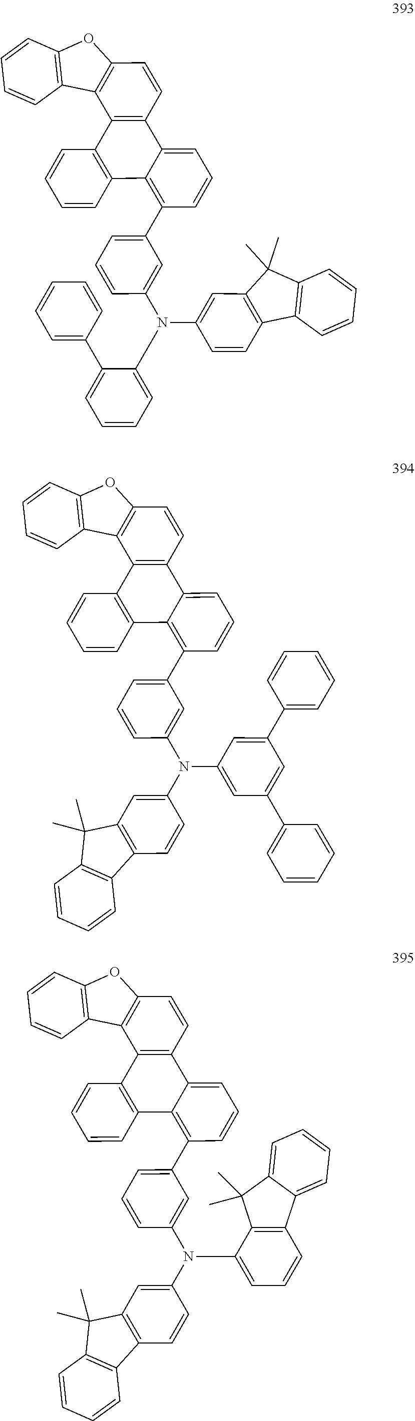 [SMILES: CC1(C)c2ccccc2-c2ccc(N(c3cccc(-c4cccc5c6ccc7oc8ccccc8c7c6c6ccccc6c45)c3)c3ccccc3-c3ccccc3)cc21 ; CC1(C)c2ccccc2-c2ccc(N(c3cc(-c4ccccc4)cc(-c4ccccc4)c3)c3cccc(-c4cccc5c6ccc7oc8ccccc8c7c6c6ccccc6c45)c3)cc21 ; CC1(C)c2ccccc2-c2ccc(N(c3cccc(-c4cccc5c6ccc7oc8ccccc8c7c6c6ccccc6c45)c3)c3cccc4c3C(C)(C)c3ccccc3-4)cc21]

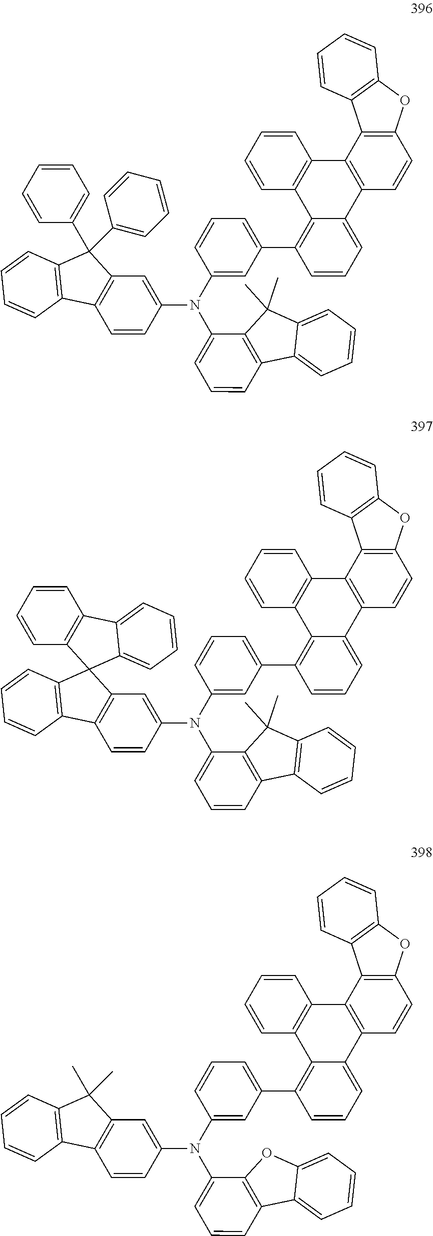 [SMILES: CC1(C)c2ccccc2-c2cccc(N(c3cccc(-c4cccc5c6ccc7oc8ccccc8c7c6c6ccccc6c45)c3)c3ccc4c(c3)C(c3ccccc3)(c3ccccc3)c3ccccc3-4)c21 ; CC1(C)c2ccccc2-c2cccc(N(c3cccc(-c4cccc5c6ccc7oc8ccccc8c7c6c6ccccc6c45)c3)c3ccc4c(c3)C3(c5ccccc5-c5ccccc53)c3ccccc3-4)c21 ; CC1(C)c2ccccc2-c2ccc(N(c3cccc(-c4cccc5c6ccc7oc8ccccc8c7c6c6ccccc6c45)c3)c3cccc4c3oc3ccccc34)cc21]

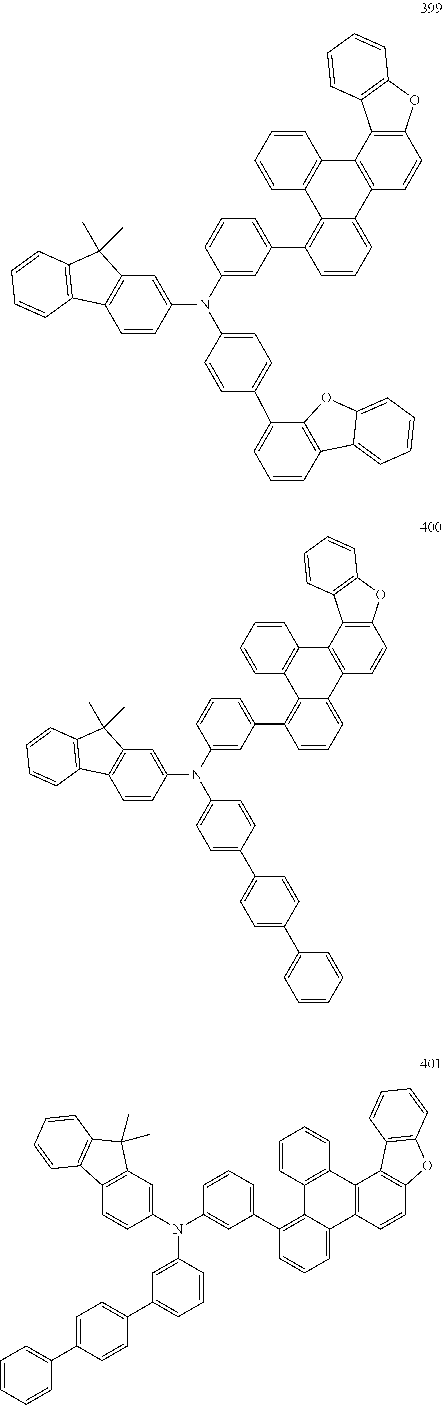 [SMILES: CC1(C)c2ccccc2-c2ccc(N(c3ccc(-c4cccc5c4oc4ccccc45)cc3)c3cccc(-c4cccc5c6ccc7oc8ccccc8c7c6c6ccccc6c45)c3)cc21 ; CC1(C)c2ccccc2-c2ccc(N(c3ccc(-c4ccc(-c5ccccc5)cc4)cc3)c3cccc(-c4cccc5c6ccc7oc8ccccc8c7c6c6ccccc6c45)c3)cc21 ; CC1(C)c2ccccc2-c2ccc(N(c3cccc(-c4ccc(-c5ccccc5)cc4)c3)c3cccc(-c4cccc5c6ccc7oc8ccccc8c7c6c6ccccc6c45)c3)cc21]

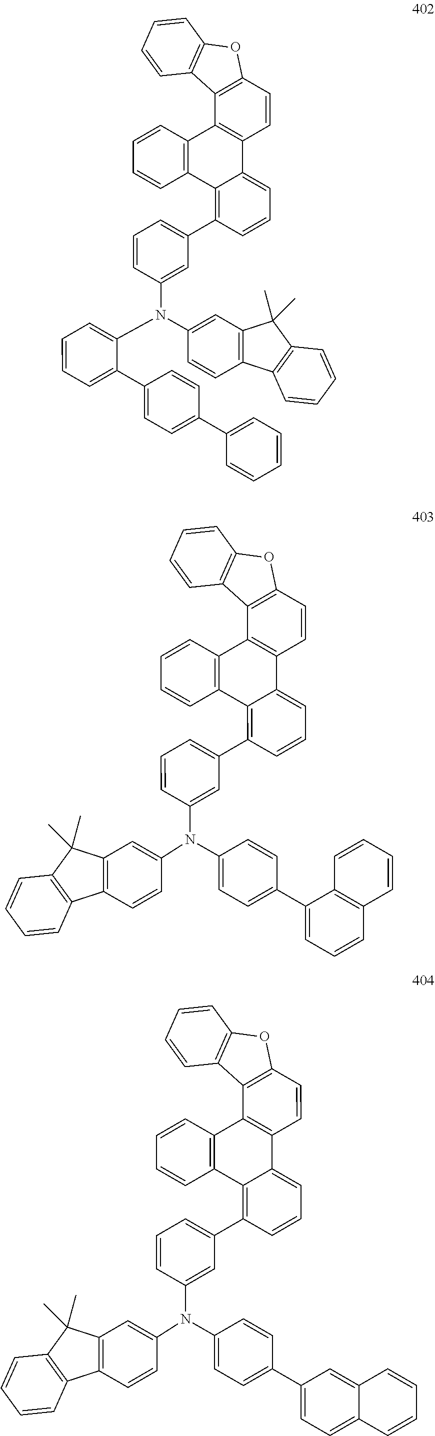 [SMILES: CC1(C)c2ccccc2-c2ccc(N(c3cccc(-c4cccc5c6ccc7oc8ccccc8c7c6c6ccccc6c45)c3)c3ccccc3-c3ccc(-c4ccccc4)cc3)cc21 ; CC1(C)c2ccccc2-c2ccc(N(c3ccc(-c4cccc5ccccc45)cc3)c3cccc(-c4cccc5c6ccc7oc8ccccc8c7c6c6ccccc6c45)c3)cc21 ; CC1(C)c2ccccc2-c2ccc(N(c3ccc(-c4ccc5ccccc5c4)cc3)c3cccc(-c4cccc5c6ccc7oc8ccccc8c7c6c6ccccc6c45)c3)cc21]

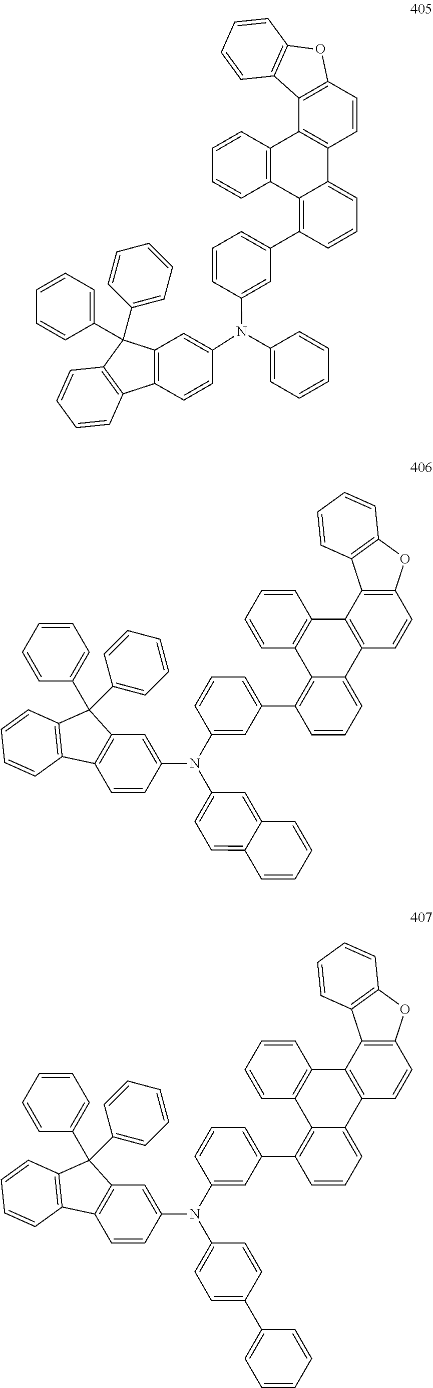 [SMILES: c1ccc(N(c2cccc(-c3cccc4c5ccc6oc7ccccc7c6c5c5ccccc5c34)c2)c2ccc3c(c2)C(c2ccccc2)(c2ccccc2)c2ccccc2-3)cc1 ; c1ccc(C2(c3ccccc3)c3ccccc3-c3ccc(N(c4cccc(-c5cccc6c7ccc8oc9ccccc9c8c7c7ccccc7c56)c4)c4ccc5ccccc5c4)cc32)cc1 ; c1ccc(-c2ccc(N(c3cccc(-c4cccc5c6ccc7oc8ccccc8c7c6c6ccccc6c45)c3)c3ccc4c(c3)C(c3ccccc3)(c3ccccc3)c3ccccc3-4)cc2)cc1]

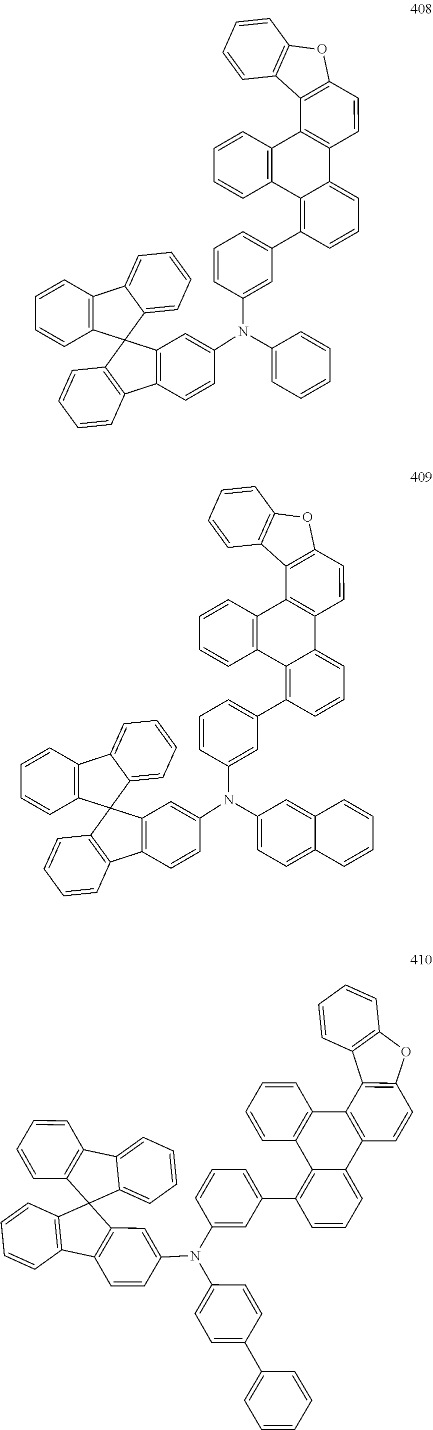 [SMILES: c1ccc(N(c2cccc(-c3cccc4c5ccc6oc7ccccc7c6c5c5ccccc5c34)c2)c2ccc3c(c2)C2(c4ccccc4-c4ccccc42)c2ccccc2-3)cc1 ; c1cc(-c2cccc3c4ccc5oc6ccccc6c5c4c4ccccc4c23)cc(N(c2ccc3c(c2)C2(c4ccccc4-c4ccccc42)c2ccccc2-3)c2ccc3ccccc3c2)c1 ; c1ccc(-c2ccc(N(c3cccc(-c4cccc5c6ccc7oc8ccccc8c7c6c6ccccc6c45)c3)c3ccc4c(c3)C3(c5ccccc5-c5ccccc53)c3ccccc3-4)cc2)cc1]

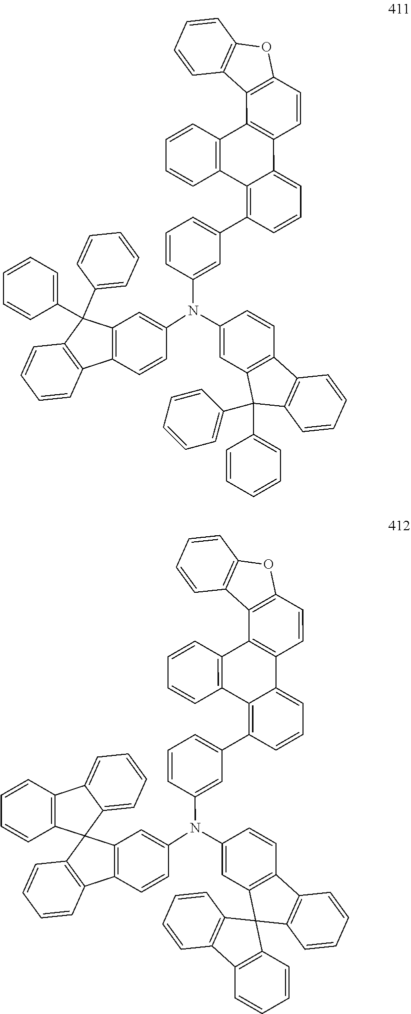 [SMILES: c1ccc(C2(c3ccccc3)c3ccccc3-c3ccc(N(c4cccc(-c5cccc6c7ccc8oc9ccccc9c8c7c7ccccc7c56)c4)c4ccc5c(c4)C(c4ccccc4)(c4ccccc4)c4ccccc4-5)cc32)cc1 ; c1cc(-c2cccc3c4ccc5oc6ccccc6c5c4c4ccccc4c23)cc(N(c2ccc3c(c2)C2(c4ccccc4-c4ccccc42)c2ccccc2-3)c2ccc3c(c2)C2(c4ccccc4-c4ccccc42)c2ccccc2-3)c1]

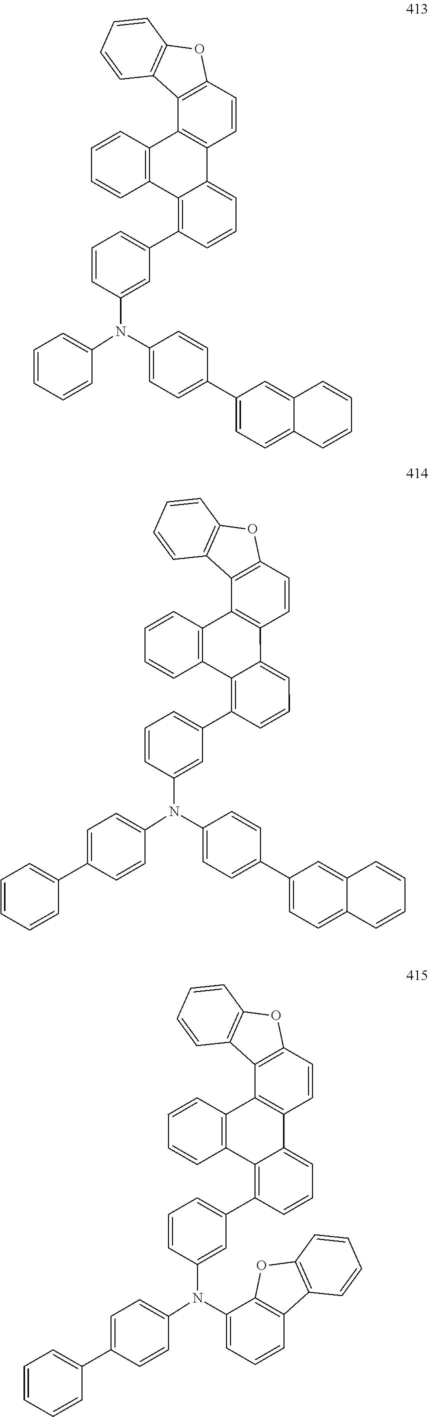 [SMILES: c1ccc(N(c2ccc(-c3ccc4ccccc4c3)cc2)c2cccc(-c3cccc4c5ccc6oc7ccccc7c6c5c5ccccc5c34)c2)cc1 ; c1ccc(-c2ccc(N(c3ccc(-c4ccc5ccccc5c4)cc3)c3cccc(-c4cccc5c6ccc7oc8ccccc8c7c6c6ccccc6c45)c3)cc2)cc1 ; c1ccc(-c2ccc(N(c3cccc(-c4cccc5c6ccc7oc8ccccc8c7c6c6ccccc6c45)c3)c3cccc4c3oc3ccccc34)cc2)cc1]

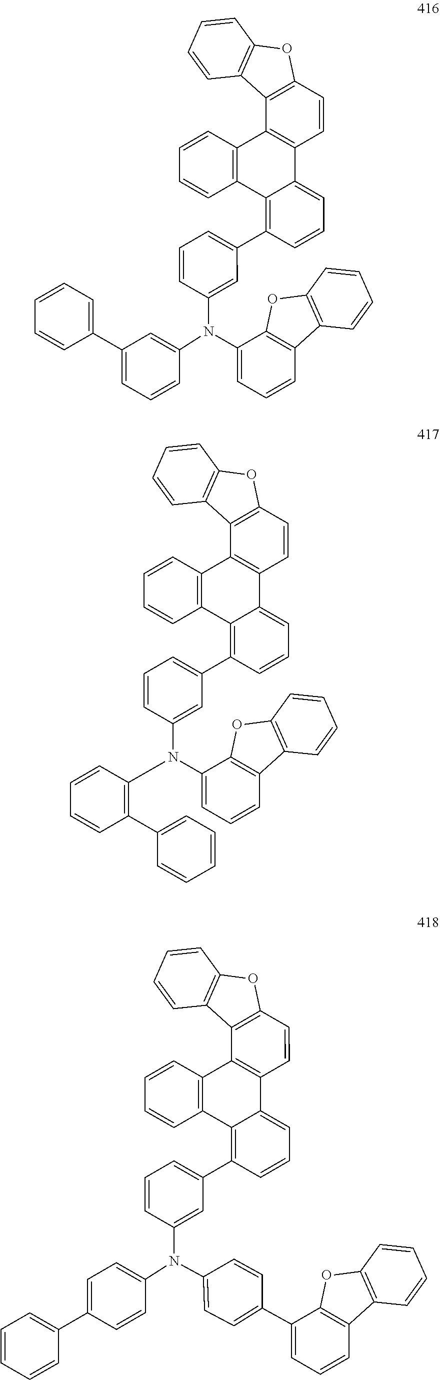 [SMILES: c1ccc(-c2cccc(N(c3cccc(-c4cccc5c6ccc7oc8ccccc8c7c6c6ccccc6c45)c3)c3cccc4c3oc3ccccc34)c2)cc1 ; c1ccc(-c2ccccc2N(c2cccc(-c3cccc4c5ccc6oc7ccccc7c6c5c5ccccc5c34)c2)c2cccc3c2oc2ccccc23)cc1 ; c1ccc(-c2ccc(N(c3ccc(-c4cccc5c4oc4ccccc45)cc3)c3cccc(-c4cccc5c6ccc7oc8ccccc8c7c6c6ccccc6c45)c3)cc2)cc1]

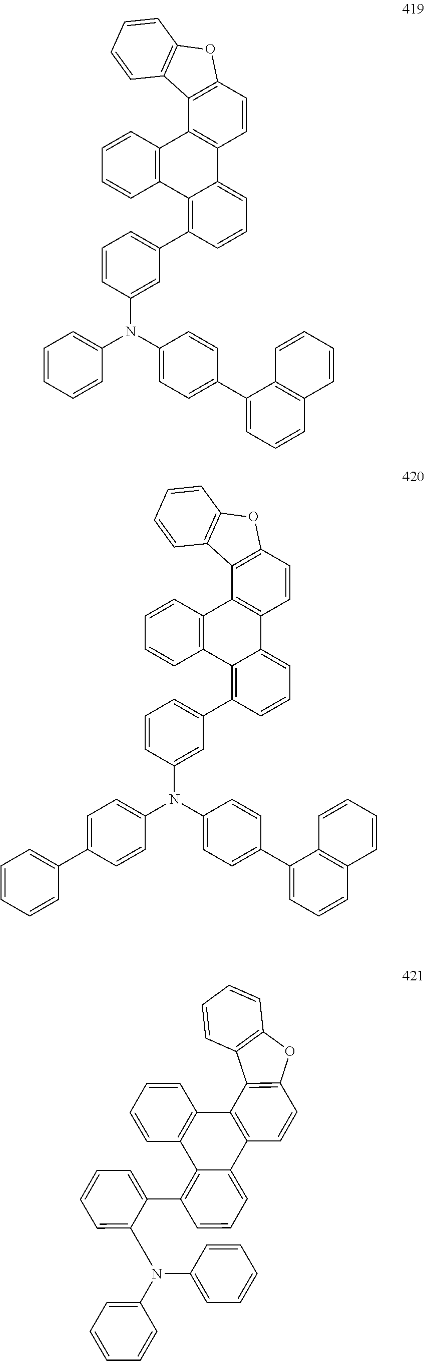 [SMILES: c1ccc(N(c2ccc(-c3cccc4ccccc34)cc2)c2cccc(-c3cccc4c5ccc6oc7ccccc7c6c5c5ccccc5c34)c2)cc1 ; c1ccc(-c2ccc(N(c3ccc(-c4cccc5ccccc45)cc3)c3cccc(-c4cccc5c6ccc7oc8ccccc8c7c6c6ccccc6c45)c3)cc2)cc1 ; c1ccc(N(c2ccccc2)c2ccccc2-c2cccc3c4ccc5oc6ccccc6c5c4c4ccccc4c23)cc1]

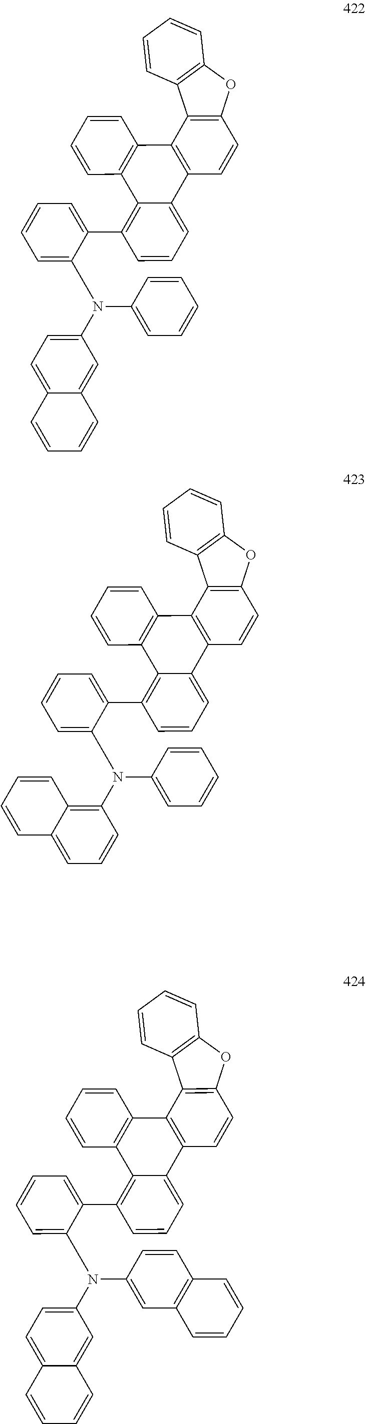 [SMILES: c1ccc(N(c2ccc3ccccc3c2)c2ccccc2-c2cccc3c4ccc5oc6ccccc6c5c4c4ccccc4c23)cc1 ; c1ccc(N(c2ccccc2-c2cccc3c4ccc5oc6ccccc6c5c4c4ccccc4c23)c2cccc3ccccc23)cc1 ; c1ccc(N(c2ccc3ccccc3c2)c2ccc3ccccc3c2)c(-c2cccc3c4ccc5oc6ccccc6c5c4c4ccccc4c23)c1]

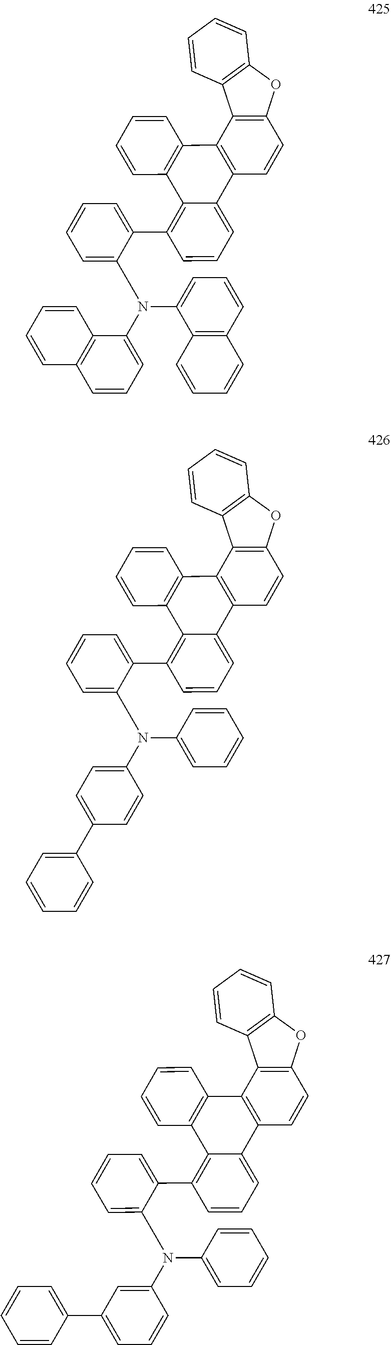 [SMILES: c1ccc(N(c2cccc3ccccc23)c2cccc3ccccc23)c(-c2cccc3c4ccc5oc6ccccc6c5c4c4ccccc4c23)c1 ; c1ccc(-c2ccc(N(c3ccccc3)c3ccccc3-c3cccc4c5ccc6oc7ccccc7c6c5c5ccccc5c34)cc2)cc1 ; c1ccc(-c2cccc(N(c3ccccc3)c3ccccc3-c3cccc4c5ccc6oc7ccccc7c6c5c5ccccc5c34)c2)cc1]

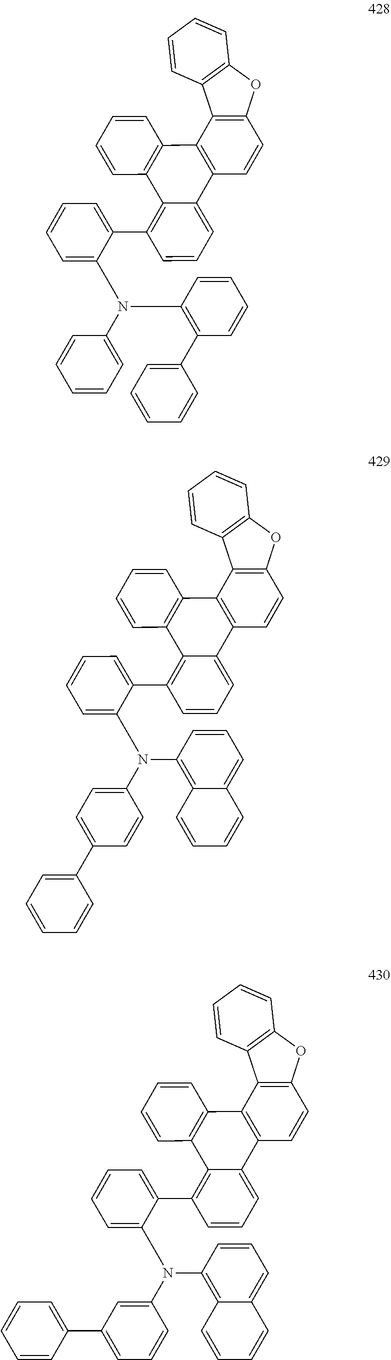 [SMILES: c1ccc(-c2ccccc2N(c2ccccc2)c2ccccc2-c2cccc3c4ccc5oc6ccccc6c5c4c4ccccc4c23)cc1 ; c1ccc(-c2ccc(N(c3ccccc3-c3cccc4c5ccc6oc7ccccc7c6c5c5ccccc5c34)c3cccc4ccccc34)cc2)cc1 ; c1ccc(-c2cccc(N(c3ccccc3-c3cccc4c5ccc6oc7ccccc7c6c5c5ccccc5c34)c3cccc4ccccc34)c2)cc1]

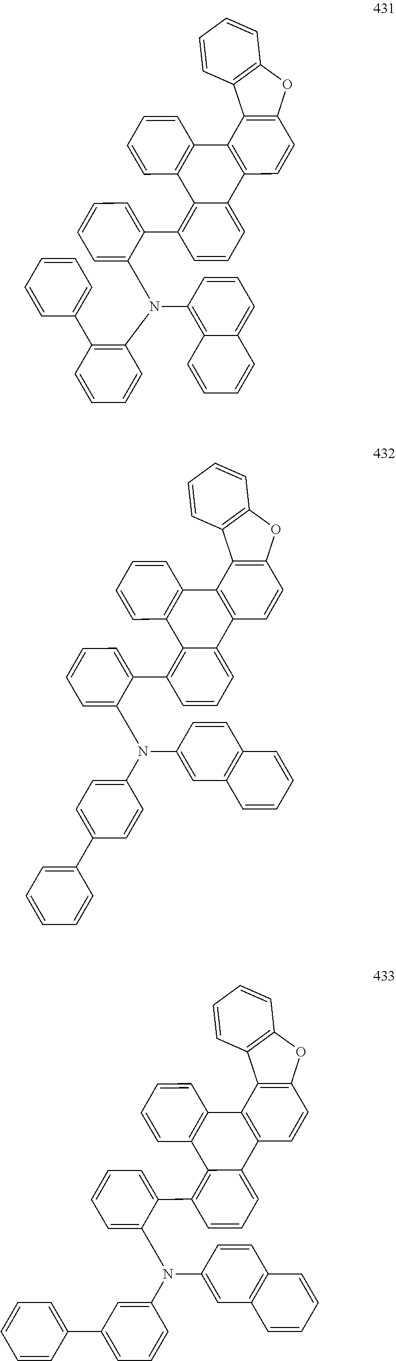 [SMILES: c1ccc(-c2ccccc2N(c2ccccc2-c2cccc3c4ccc5oc6ccccc6c5c4c4ccccc4c23)c2cccc3ccccc23)cc1 ; c1ccc(-c2ccc(N(c3ccc4ccccc4c3)c3ccccc3-c3cccc4c5ccc6oc7ccccc7c6c5c5ccccc5c34)cc2)cc1 ; c1ccc(-c2cccc(N(c3ccc4ccccc4c3)c3ccccc3-c3cccc4c5ccc6oc7ccccc7c6c5c5ccccc5c34)c2)cc1]

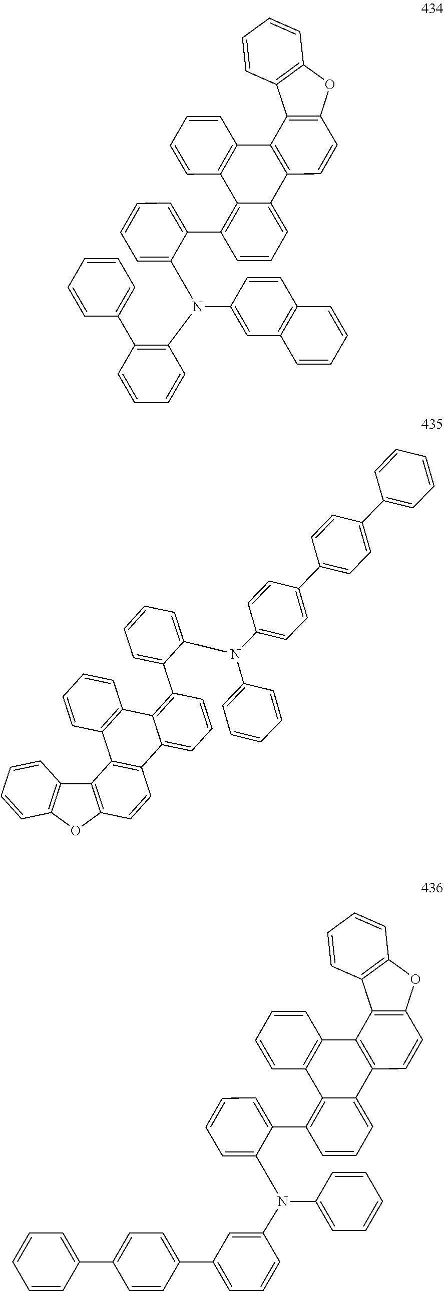 [SMILES: c1ccc(-c2ccccc2N(c2ccc3ccccc3c2)c2ccccc2-c2cccc3c4ccc5oc6ccccc6c5c4c4ccccc4c23)cc1 ; c1ccc(-c2ccc(-c3ccc(N(c4ccccc4)c4ccccc4-c4cccc5c6ccc7oc8ccccc8c7c6c6ccccc6c45)cc3)cc2)cc1 ; c1ccc(-c2ccc(-c3cccc(N(c4ccccc4)c4ccccc4-c4cccc5c6ccc7oc8ccccc8c7c6c6ccccc6c45)c3)cc2)cc1]

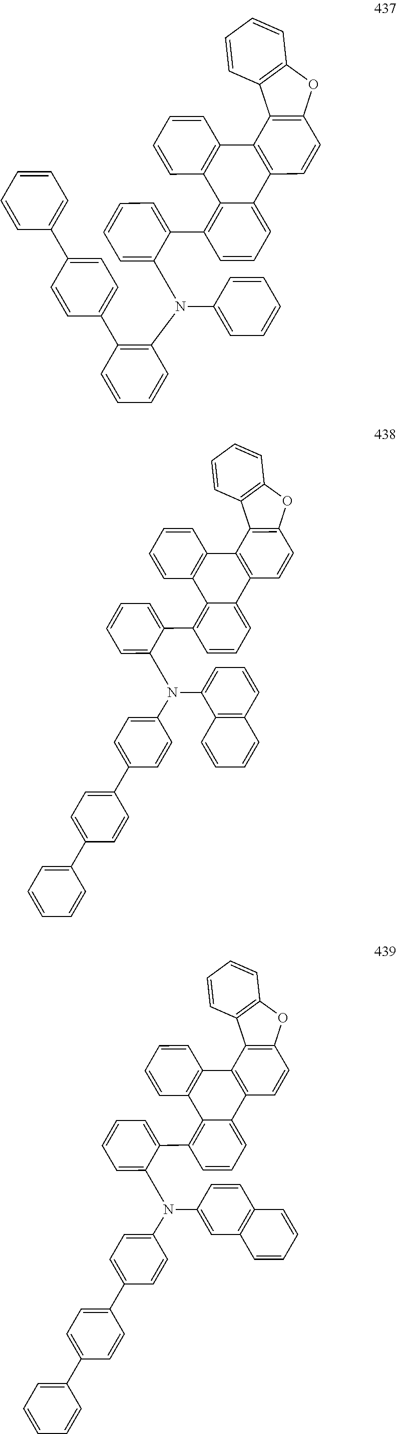 [SMILES: c1ccc(-c2ccc(-c3ccccc3N(c3ccccc3)c3ccccc3-c3cccc4c5ccc6oc7ccccc7c6c5c5ccccc5c34)cc2)cc1 ; c1ccc(-c2ccc(-c3ccc(N(c4ccccc4-c4cccc5c6ccc7oc8ccccc8c7c6c6ccccc6c45)c4cccc5ccccc45)cc3)cc2)cc1 ; c1ccc(-c2ccc(-c3ccc(N(c4ccc5ccccc5c4)c4ccccc4-c4cccc5c6ccc7oc8ccccc8c7c6c6ccccc6c45)cc3)cc2)cc1]

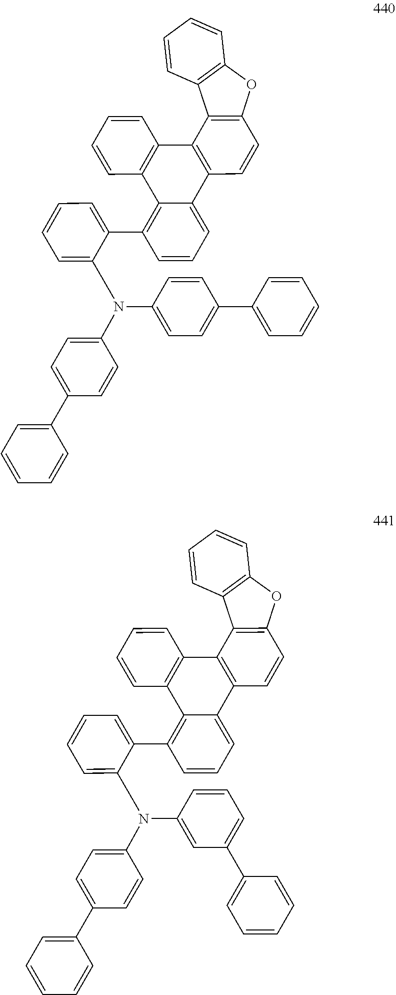 [SMILES: c1ccc(-c2ccc(N(c3ccc(-c4ccccc4)cc3)c3ccccc3-c3cccc4c5ccc6oc7ccccc7c6c5c5ccccc5c34)cc2)cc1 ; c1ccc(-c2ccc(N(c3cccc(-c4ccccc4)c3)c3ccccc3-c3cccc4c5ccc6oc7ccccc7c6c5c5ccccc5c34)cc2)cc1]

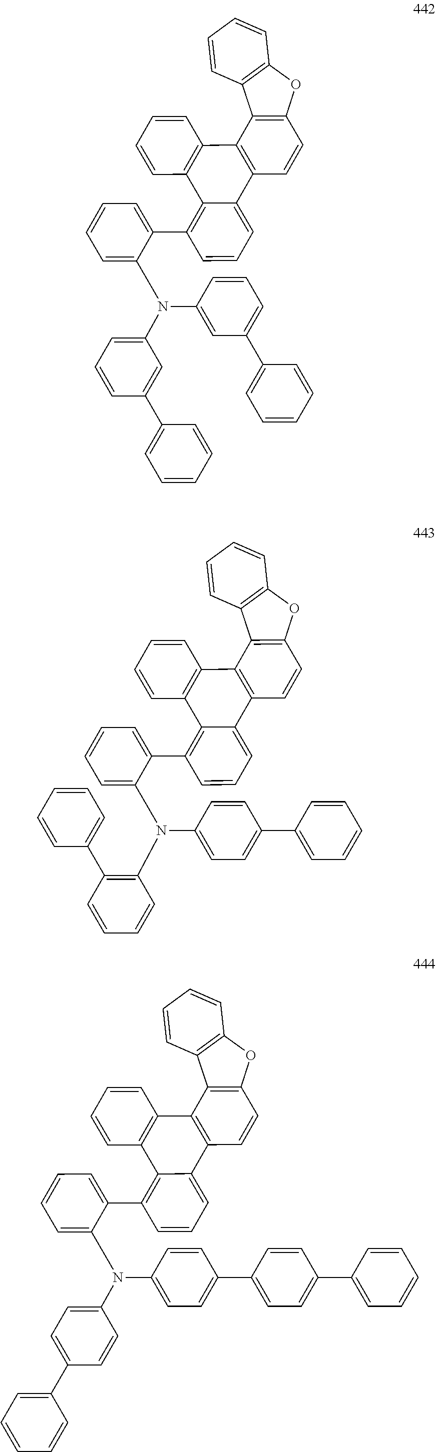 [SMILES: c1ccc(-c2cccc(N(c3cccc(-c4ccccc4)c3)c3ccccc3-c3cccc4c5ccc6oc7ccccc7c6c5c5ccccc5c34)c2)cc1 ; c1ccc(-c2ccc(N(c3ccccc3-c3ccccc3)c3ccccc3-c3cccc4c5ccc6oc7ccccc7c6c5c5ccccc5c34)cc2)cc1 ; c1ccc(-c2ccc(-c3ccc(N(c4ccc(-c5ccccc5)cc4)c4ccccc4-c4cccc5c6ccc7oc8ccccc8c7c6c6ccccc6c45)cc3)cc2)cc1]

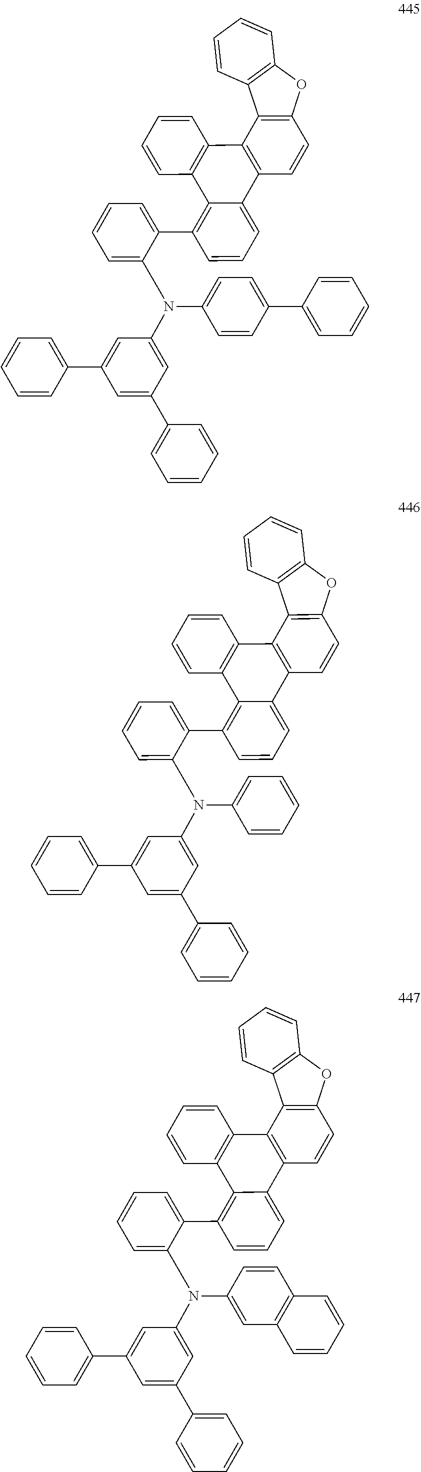

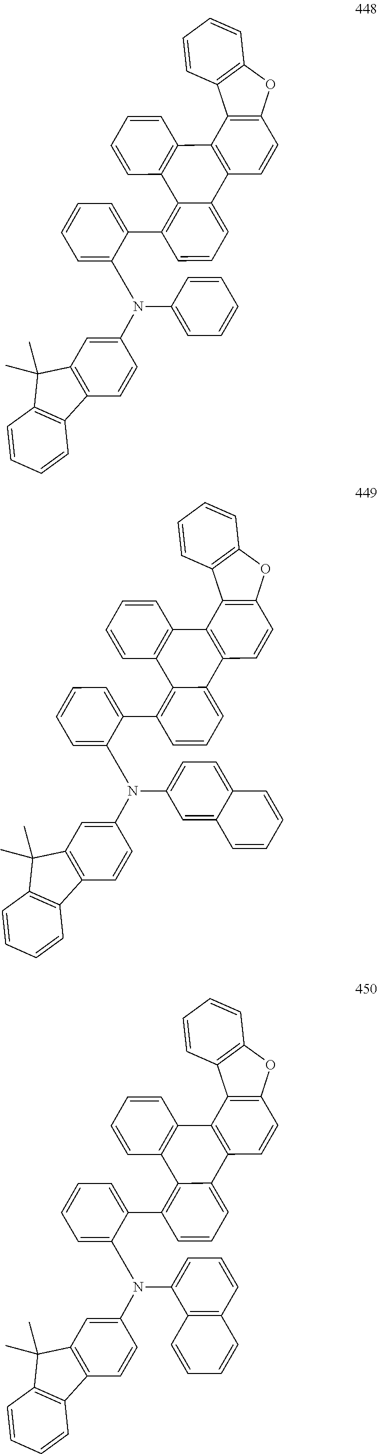 [SMILES: CC1(C)c2ccccc2-c2ccc(N(c3ccccc3)c3ccccc3-c3cccc4c5ccc6oc7ccccc7c6c5c5ccccc5c34)cc21 ; CC1(C)c2ccccc2-c2ccc(N(c3ccc4ccccc4c3)c3ccccc3-c3cccc4c5ccc6oc7ccccc7c6c5c5ccccc5c34)cc21 ; CC1(C)c2ccccc2-c2ccc(N(c3ccccc3-c3cccc4c5ccc6oc7ccccc7c6c5c5ccccc5c34)c3cccc4ccccc34)cc21]

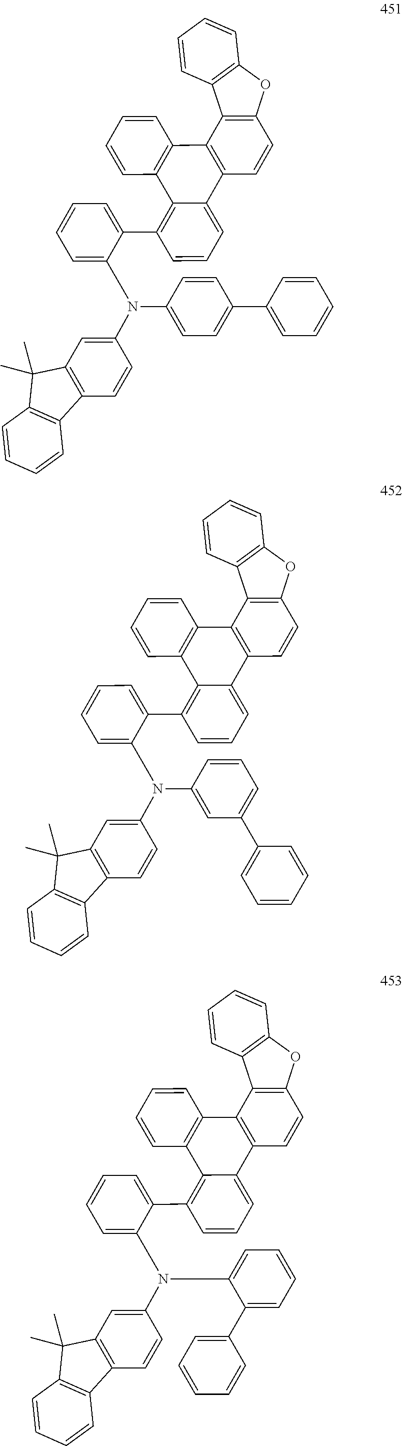 [SMILES: CC1(C)c2ccccc2-c2ccc(N(c3ccc(-c4ccccc4)cc3)c3ccccc3-c3cccc4c5ccc6oc7ccccc7c6c5c5ccccc5c34)cc21 ; CC1(C)c2ccccc2-c2ccc(N(c3cccc(-c4ccccc4)c3)c3ccccc3-c3cccc4c5ccc6oc7ccccc7c6c5c5ccccc5c34)cc21 ; CC1(C)c2ccccc2-c2ccc(N(c3ccccc3-c3ccccc3)c3ccccc3-c3cccc4c5ccc6oc7ccccc7c6c5c5ccccc5c34)cc21]

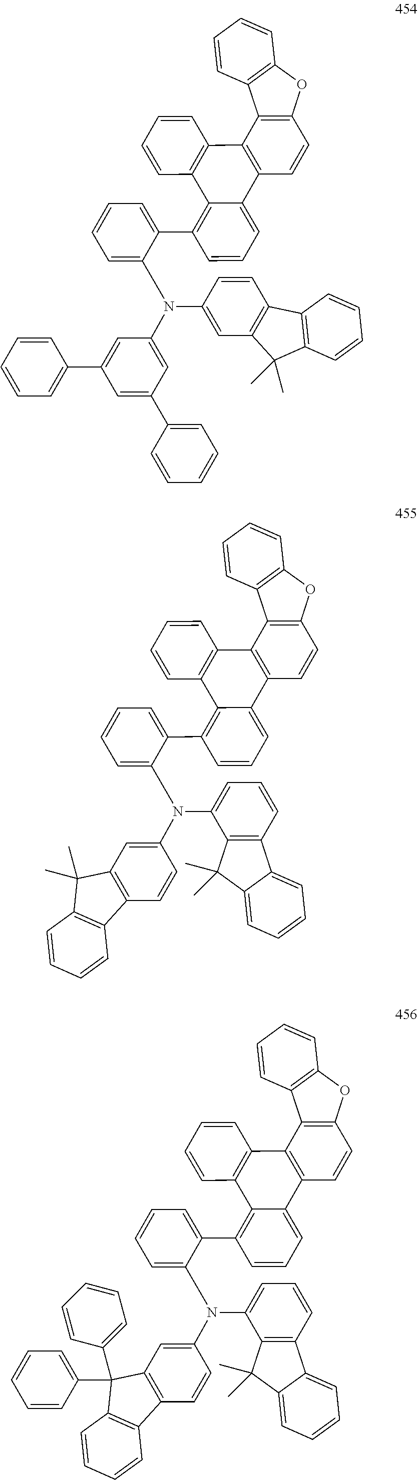 [SMILES: CC1(C)c2ccccc2-c2ccc(N(c3cc(-c4ccccc4)cc(-c4ccccc4)c3)c3ccccc3-c3cccc4c5ccc6oc7ccccc7c6c5c5ccccc5c34)cc21 ; CC1(C)c2ccccc2-c2ccc(N(c3ccccc3-c3cccc4c5ccc6oc7ccccc7c6c5c5ccccc5c34)c3cccc4c3C(C)(C)c3ccccc3-4)cc21 ; CC1(C)c2ccccc2-c2cccc(N(c3ccc4c(c3)C(c3ccccc3)(c3ccccc3)c3ccccc3-4)c3ccccc3-c3cccc4c5ccc6oc7ccccc7c6c5c5ccccc5c34)c21]

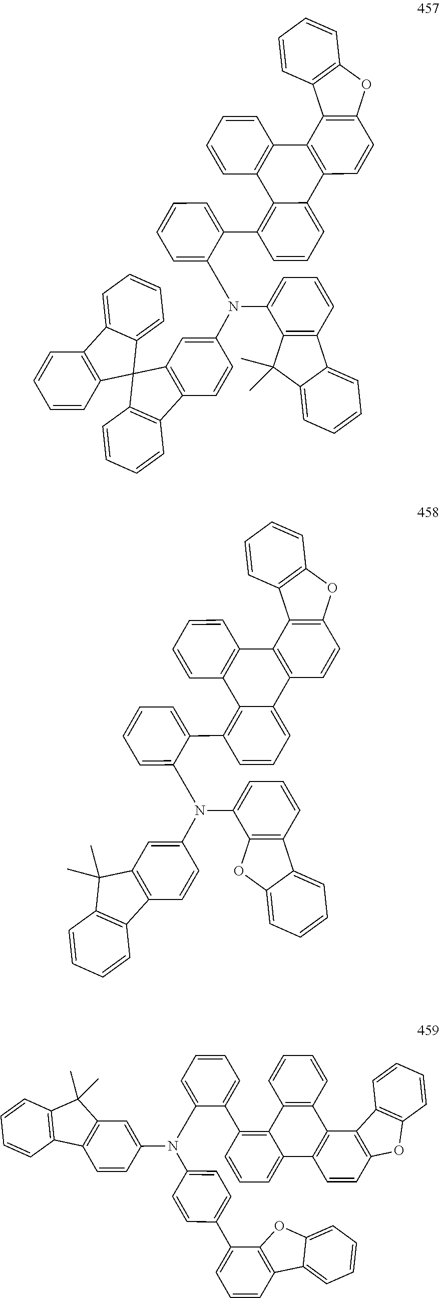 [SMILES: CC1(C)c2ccccc2-c2cccc(N(c3ccc4c(c3)C3(c5ccccc5-c5ccccc53)c3ccccc3-4)c3ccccc3-c3cccc4c5ccc6oc7ccccc7c6c5c5ccccc5c34)c21 ; CC1(C)c2ccccc2-c2ccc(N(c3ccccc3-c3cccc4c5ccc6oc7ccccc7c6c5c5ccccc5c34)c3cccc4c3oc3ccccc34)cc21 ; CC1(C)c2ccccc2-c2ccc(N(c3ccc(-c4cccc5c4oc4ccccc45)cc3)c3ccccc3-c3cccc4c5ccc6oc7ccccc7c6c5c5ccccc5c34)cc21]

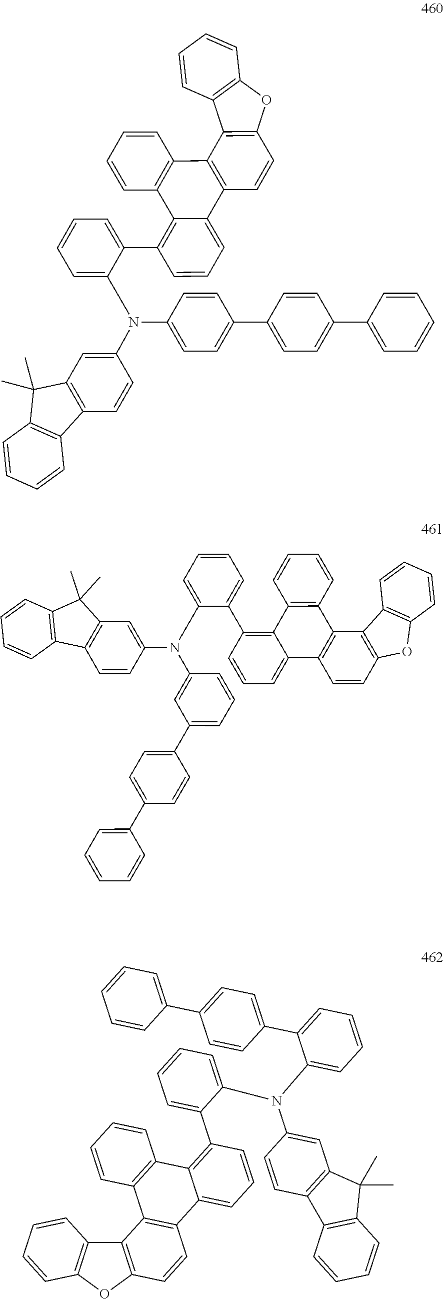 [SMILES: CC1(C)c2ccccc2-c2ccc(N(c3ccc(-c4ccc(-c5ccccc5)cc4)cc3)c3ccccc3-c3cccc4c5ccc6oc7ccccc7c6c5c5ccccc5c34)cc21 ; CC1(C)c2ccccc2-c2ccc(N(c3cccc(-c4ccc(-c5ccccc5)cc4)c3)c3ccccc3-c3cccc4c5ccc6oc7ccccc7c6c5c5ccccc5c34)cc21 ; CC1(C)c2ccccc2-c2ccc(N(c3ccccc3-c3ccc(-c4ccccc4)cc3)c3ccccc3-c3cccc4c5ccc6oc7ccccc7c6c5c5ccccc5c34)cc21]

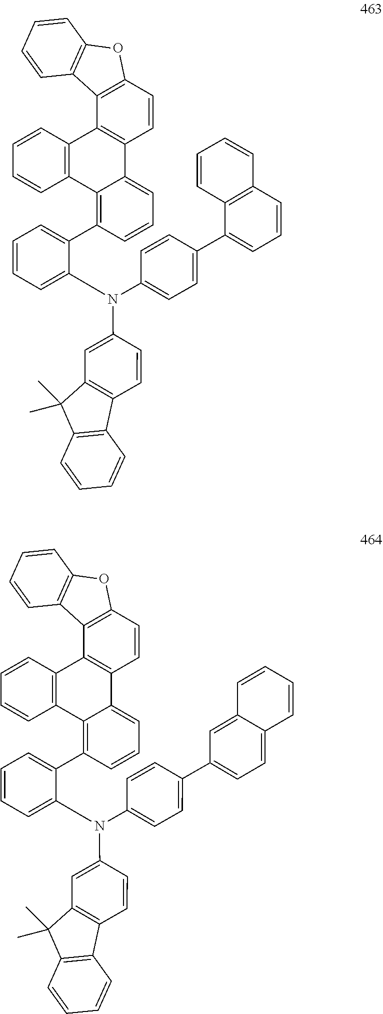 [SMILES: CC1(C)c2ccccc2-c2ccc(N(c3ccc(-c4cccc5ccccc45)cc3)c3ccccc3-c3cccc4c5ccc6oc7ccccc7c6c5c5ccccc5c34)cc21 ; CC1(C)c2ccccc2-c2ccc(N(c3ccc(-c4ccc5ccccc5c4)cc3)c3ccccc3-c3cccc4c5ccc6oc7ccccc7c6c5c5ccccc5c34)cc21]

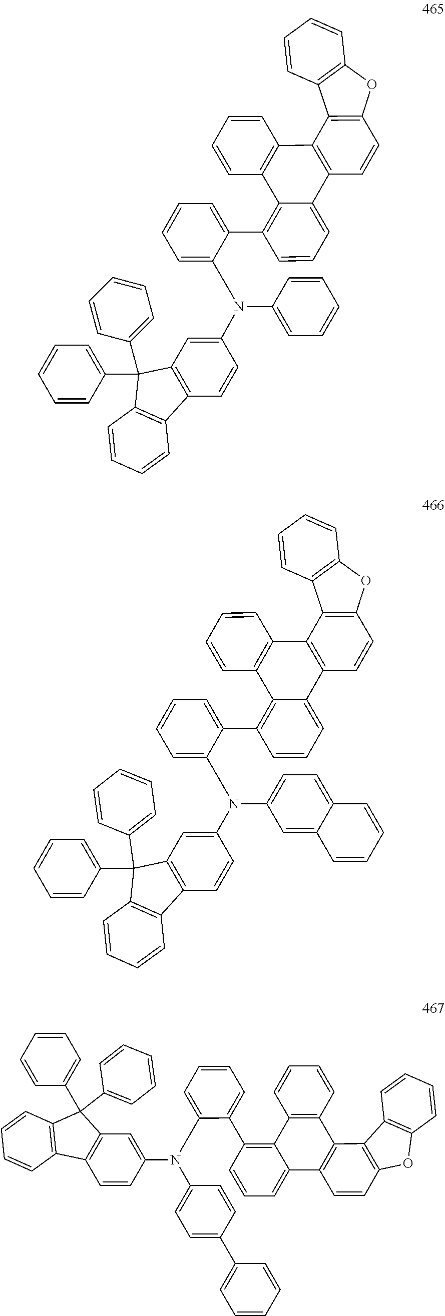 [SMILES: c1ccc(N(c2ccc3c(c2)C(c2ccccc2)(c2ccccc2)c2ccccc2-3)c2ccccc2-c2cccc3c4ccc5oc6ccccc6c5c4c4ccccc4c23)cc1 ; c1ccc(C2(c3ccccc3)c3ccccc3-c3ccc(N(c4ccc5ccccc5c4)c4ccccc4-c4cccc5c6ccc7oc8ccccc8c7c6c6ccccc6c45)cc32)cc1 ; c1ccc(-c2ccc(N(c3ccc4c(c3)C(c3ccccc3)(c3ccccc3)c3ccccc3-4)c3ccccc3-c3cccc4c5ccc6oc7ccccc7c6c5c5ccccc5c34)cc2)cc1]

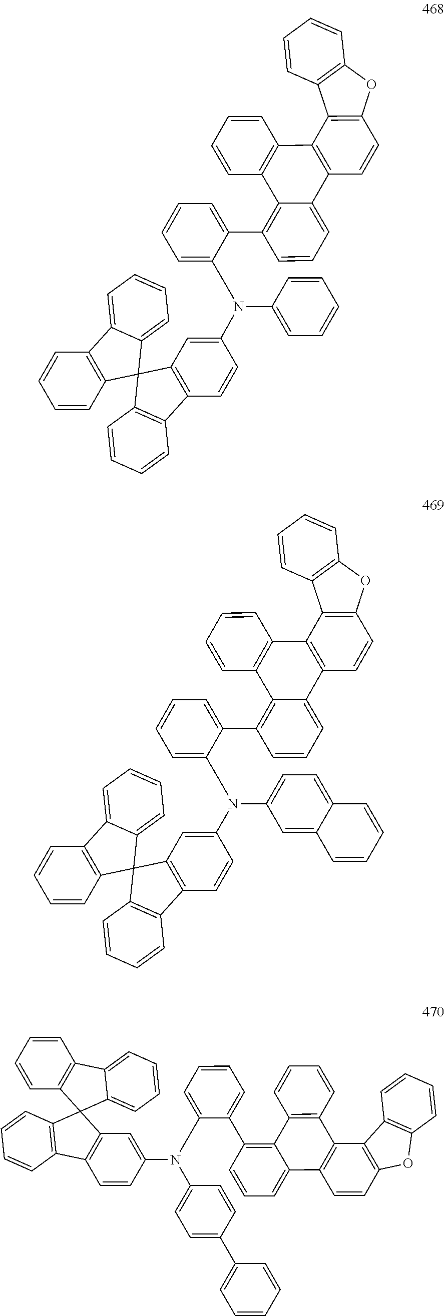 [SMILES: c1ccc(N(c2ccc3c(c2)C2(c4ccccc4-c4ccccc42)c2ccccc2-3)c2ccccc2-c2cccc3c4ccc5oc6ccccc6c5c4c4ccccc4c23)cc1 ; c1ccc(N(c2ccc3c(c2)C2(c4ccccc4-c4ccccc42)c2ccccc2-3)c2ccc3ccccc3c2)c(-c2cccc3c4ccc5oc6ccccc6c5c4c4ccccc4c23)c1 ; c1ccc(-c2ccc(N(c3ccc4c(c3)C3(c5ccccc5-c5ccccc53)c3ccccc3-4)c3ccccc3-c3cccc4c5ccc6oc7ccccc7c6c5c5ccccc5c34)cc2)cc1]

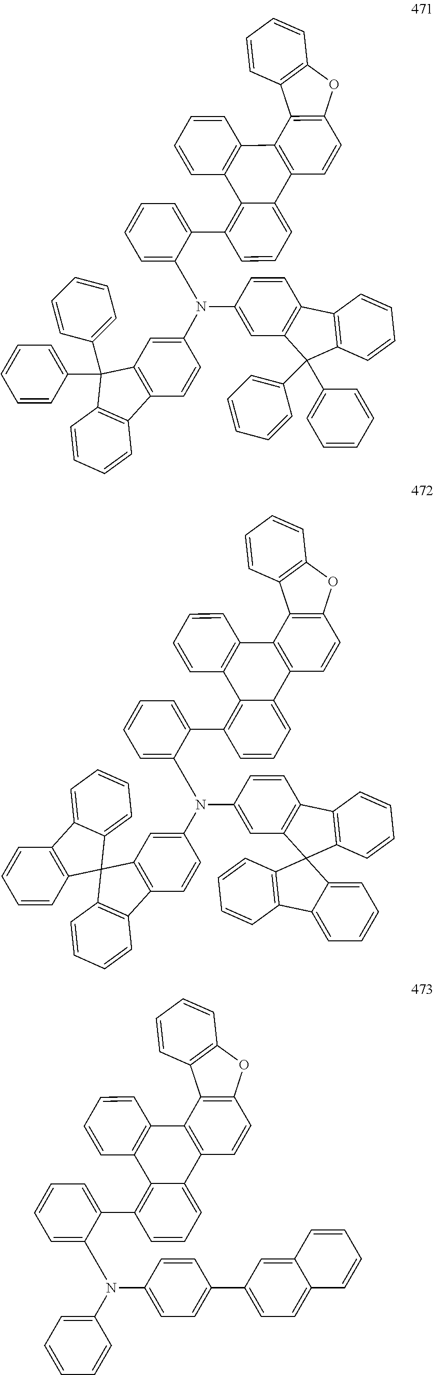 [SMILES: c1ccc(C2(c3ccccc3)c3ccccc3-c3ccc(N(c4ccc5c(c4)C(c4ccccc4)(c4ccccc4)c4ccccc4-5)c4ccccc4-c4cccc5c6ccc7oc8ccccc8c7c6c6ccccc6c45)cc32)cc1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c4ccc5oc6ccccc6c5c4c4ccccc4c23)c1 ; c1ccc(N(c2ccc(-c3ccc4ccccc4c3)cc2)c2ccccc2-c2cccc3c4ccc5oc6ccccc6c5c4c4ccccc4c23)cc1]

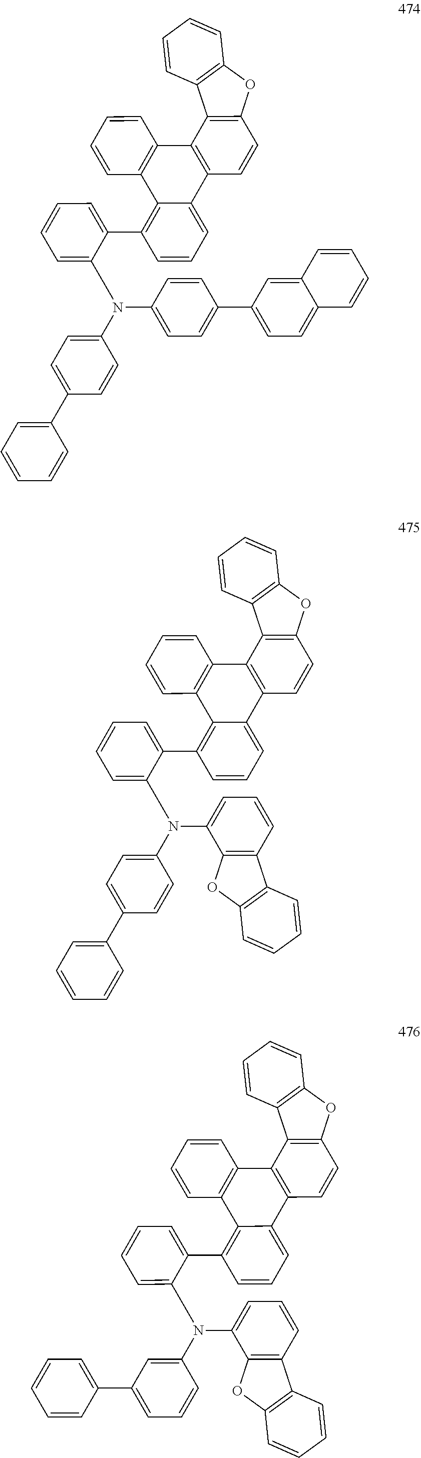 [SMILES: c1ccc(-c2ccc(N(c3ccc(-c4ccc5ccccc5c4)cc3)c3ccccc3-c3cccc4c5ccc6oc7ccccc7c6c5c5ccccc5c34)cc2)cc1 ; c1ccc(-c2ccc(N(c3ccccc3-c3cccc4c5ccc6oc7ccccc7c6c5c5ccccc5c34)c3cccc4c3oc3ccccc34)cc2)cc1 ; c1ccc(-c2cccc(N(c3ccccc3-c3cccc4c5ccc6oc7ccccc7c6c5c5ccccc5c34)c3cccc4c3oc3ccccc34)c2)cc1]

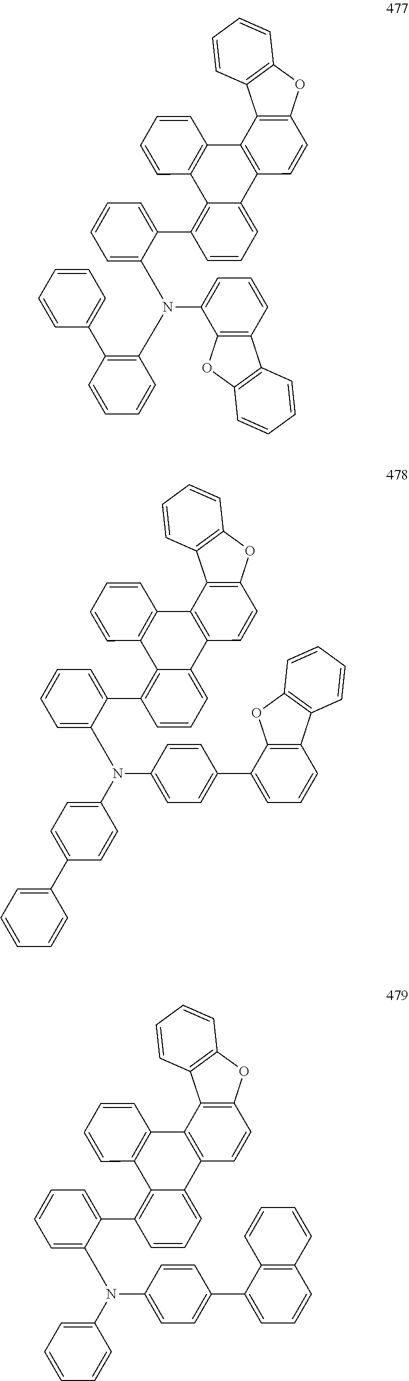 [SMILES: c1ccc(-c2ccccc2N(c2ccccc2-c2cccc3c4ccc5oc6ccccc6c5c4c4ccccc4c23)c2cccc3c2oc2ccccc23)cc1 ; c1ccc(-c2ccc(N(c3ccc(-c4cccc5c4oc4ccccc45)cc3)c3ccccc3-c3cccc4c5ccc6oc7ccccc7c6c5c5ccccc5c34)cc2)cc1 ; c1ccc(N(c2ccc(-c3cccc4ccccc34)cc2)c2ccccc2-c2cccc3c4ccc5oc6ccccc6c5c4c4ccccc4c23)cc1]

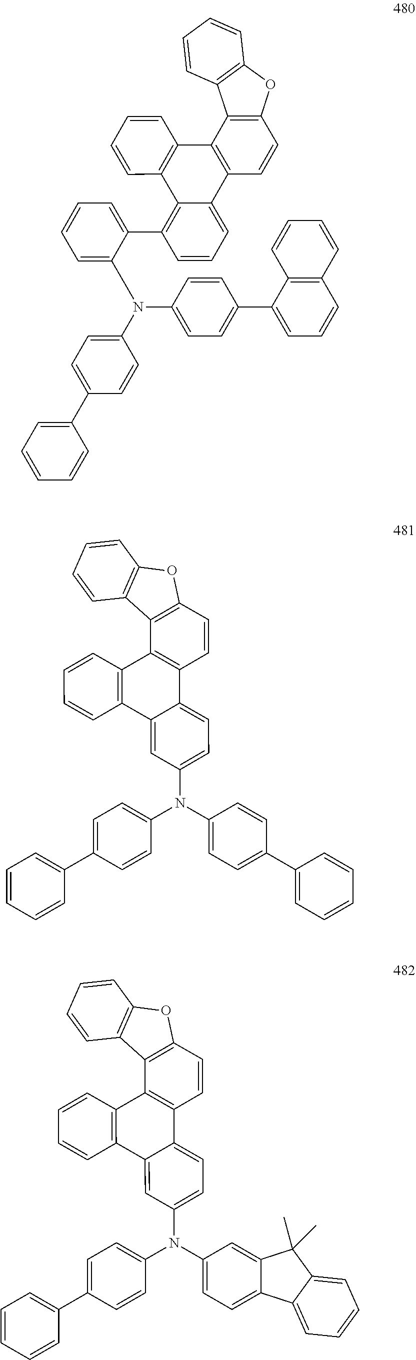 [SMILES: c1ccc(-c2ccc(N(c3ccc(-c4cccc5ccccc45)cc3)c3ccccc3-c3cccc4c5ccc6oc7ccccc7c6c5c5ccccc5c34)cc2)cc1 ; c1ccc(-c2ccc(N(c3ccc(-c4ccccc4)cc3)c3ccc4c(c3)c3ccccc3c3c4ccc4oc5ccccc5c43)cc2)cc1 ; CC1(C)c2ccccc2-c2ccc(N(c3ccc(-c4ccccc4)cc3)c3ccc4c(c3)c3ccccc3c3c4ccc4oc5ccccc5c43)cc21]

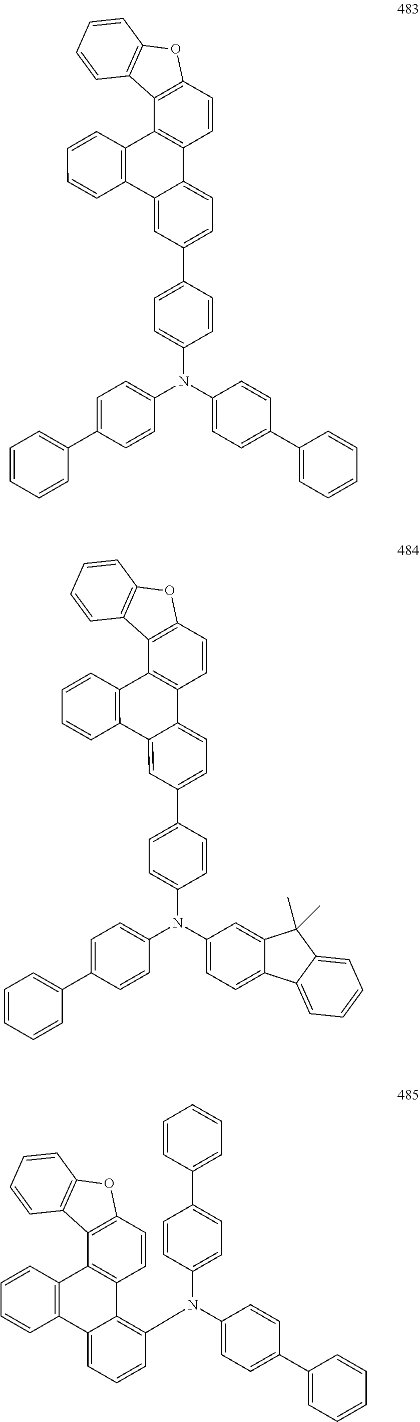 [SMILES: c1ccc(-c2ccc(N(c3ccc(-c4ccccc4)cc3)c3ccc(-c4ccc5c(c4)c4ccccc4c4c5ccc5oc6ccccc6c54)cc3)cc2)cc1 ; CC1(C)c2ccccc2-c2ccc(N(c3ccc(-c4ccccc4)cc3)c3ccc(-c4ccc5c(c4)c4ccccc4c4c5ccc5oc6ccccc6c54)cc3)cc21 ; c1ccc(-c2ccc(N(c3ccc(-c4ccccc4)cc3)c3cccc4c5ccccc5c5c(ccc6oc7ccccc7c65)c34)cc2)cc1]

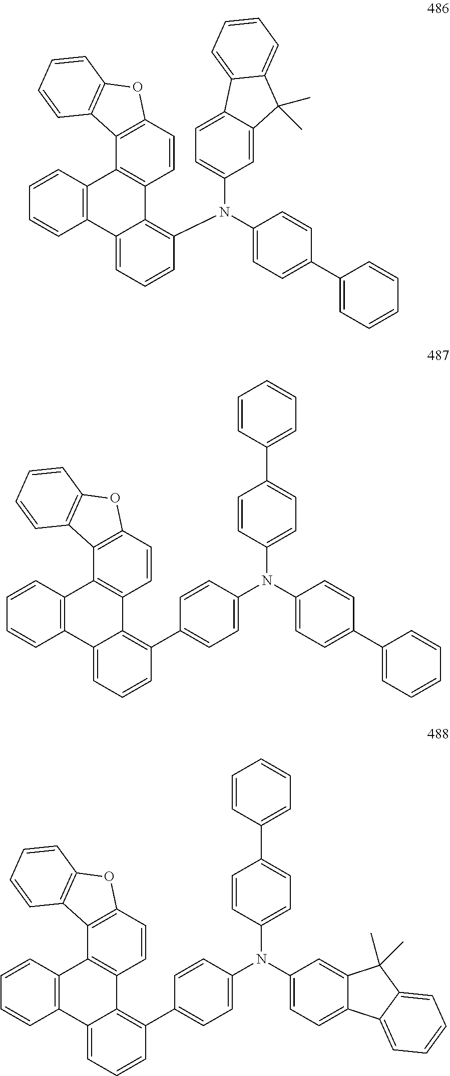 [SMILES: CC1(C)c2ccccc2-c2ccc(N(c3ccc(-c4ccccc4)cc3)c3cccc4c5ccccc5c5c(ccc6oc7ccccc7c65)c34)cc21 ; c1ccc(-c2ccc(N(c3ccc(-c4ccccc4)cc3)c3ccc(-c4cccc5c6ccccc6c6c(ccc7oc8ccccc8c76)c45)cc3)cc2)cc1 ; CC1(C)c2ccccc2-c2ccc(N(c3ccc(-c4ccccc4)cc3)c3ccc(-c4cccc5c6ccccc6c6c(ccc7oc8ccccc8c76)c45)cc3)cc21]

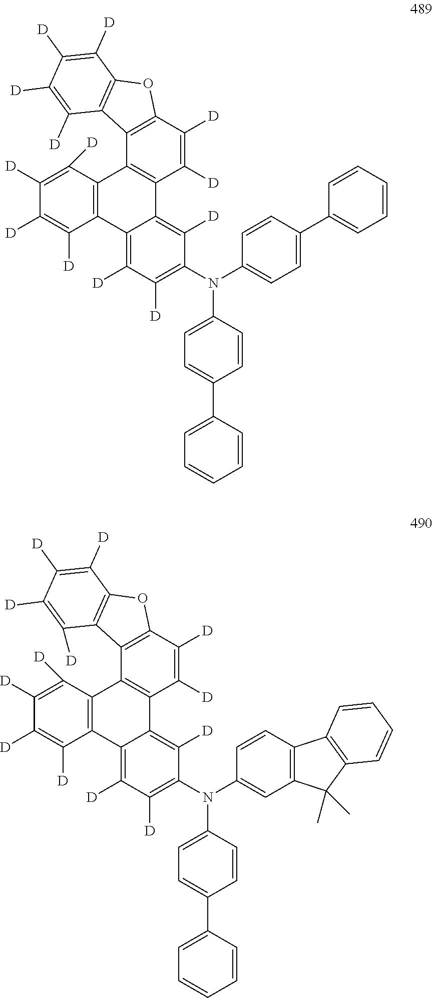 [SMILES: [2H]c1c([2H])c([2H])c2c(oc3c([2H])c([2H])c4c5c([2H])c(N(c6ccc(-c7ccccc7)cc6)c6ccc(-c7ccccc7)cc6)c([2H])c([2H])c5c5c([2H])c([2H])c([2H])c([2H])c5c4c32)c1[2H] ; [2H]c1c([2H])c([2H])c2c(oc3c([2H])c([2H])c4c5c([2H])c(N(c6ccc(-c7ccccc7)cc6)c6ccc7c(c6)C(C)(C)c6ccccc6-7)c([2H])c([2H])c5c5c([2H])c([2H])c([2H])c([2H])c5c4c32)c1[2H]]

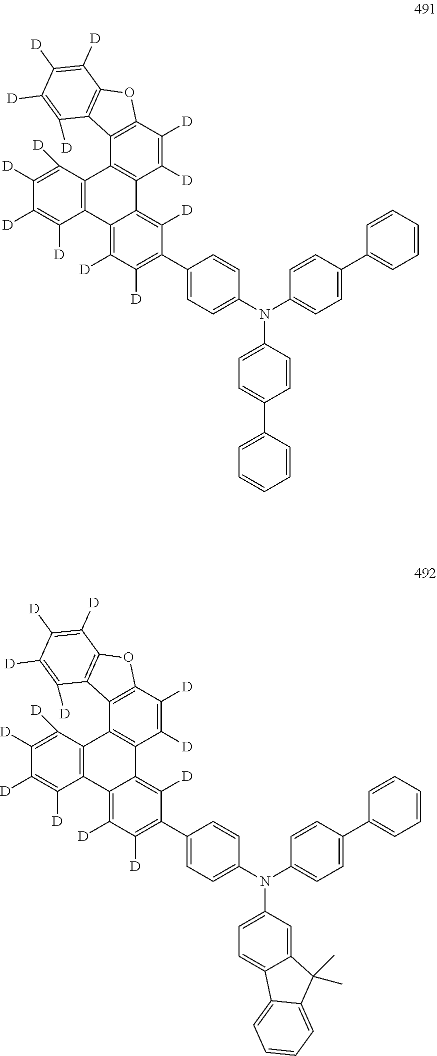 [SMILES: [2H]c1c([2H])c([2H])c2c(oc3c([2H])c([2H])c4c5c([2H])c(-c6ccc(N(c7ccc(-c8ccccc8)cc7)c7ccc(-c8ccccc8)cc7)cc6)c([2H])c([2H])c5c5c([2H])c([2H])c([2H])c([2H])c5c4c32)c1[2H] ; [2H]c1c([2H])c([2H])c2c(oc3c([2H])c([2H])c4c5c([2H])c(-c6ccc(N(c7ccc(-c8ccccc8)cc7)c7ccc8c(c7)C(C)(C)c7ccccc7-8)cc6)c([2H])c([2H])c5c5c([2H])c([2H])c([2H])c([2H])c5c4c32)c1[2H]]

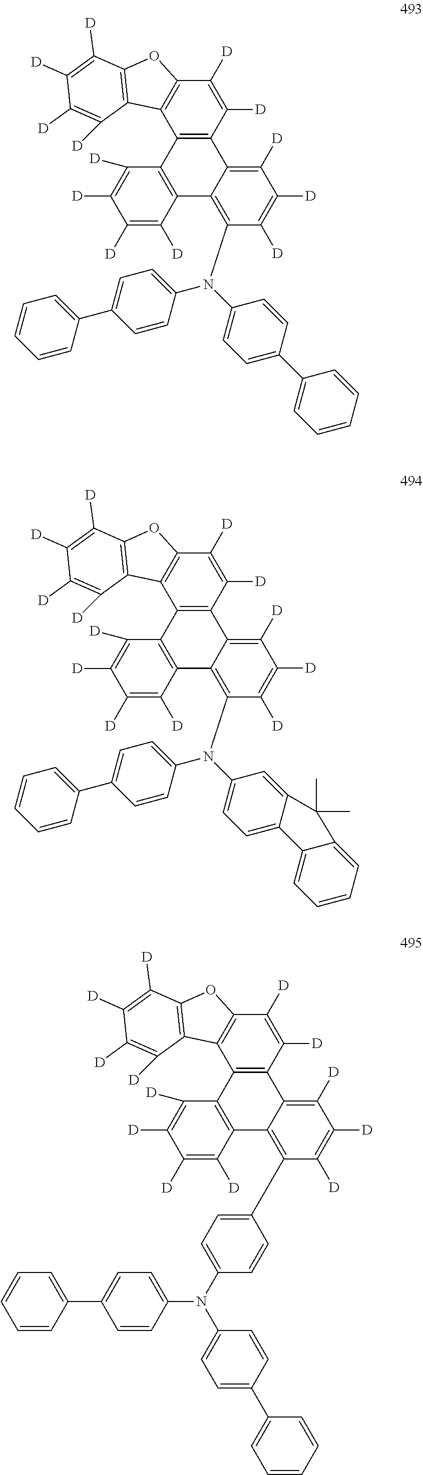 [SMILES: [2H]c1c([2H])c([2H])c2c(oc3c([2H])c([2H])c4c5c([2H])c([2H])c([2H])c(N(c6ccc(-c7ccccc7)cc6)c6ccc(-c7ccccc7)cc6)c5c5c([2H])c([2H])c([2H])c([2H])c5c4c32)c1[2H] ; [2H]c1c([2H])c([2H])c2c(oc3c([2H])c([2H])c4c5c([2H])c([2H])c([2H])c(N(c6ccc(-c7ccccc7)cc6)c6ccc7c(c6)C(C)(C)c6ccccc6-7)c5c5c([2H])c([2H])c([2H])c([2H])c5c4c32)c1[2H] ; [2H]c1c([2H])c([2H])c2c(oc3c([2H])c([2H])c4c5c([2H])c([2H])c([2H])c(-c6ccc(N(c7ccc(-c8ccccc8)cc7)c7ccc(-c8ccccc8)cc7)cc6)c5c5c([2H])c([2H])c([2H])c([2H])c5c4c32)c1[2H]]

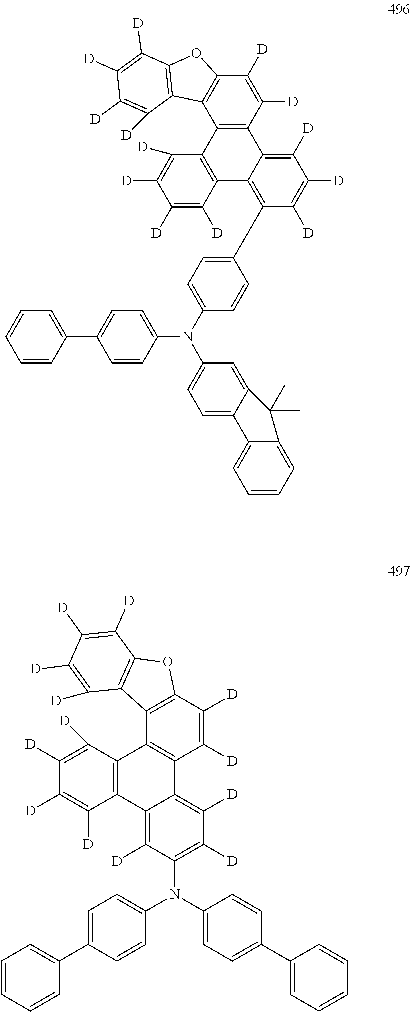 [SMILES: [2H]c1c([2H])c([2H])c2c(oc3c([2H])c([2H])c4c5c([2H])c([2H])c([2H])c(-c6ccc(N(c7ccc(-c8ccccc8)cc7)c7ccc8c(c7)C(C)(C)c7ccccc7-8)cc6)c5c5c([2H])c([2H])c([2H])c([2H])c5c4c32)c1[2H] ; [2H]c1c([2H])c([2H])c2c(oc3c([2H])c([2H])c4c5c([2H])c([2H])c(N(c6ccc(-c7ccccc7)cc6)c6ccc(-c7ccccc7)cc6)c([2H])c5c5c([2H])c([2H])c([2H])c([2H])c5c4c32)c1[2H]]

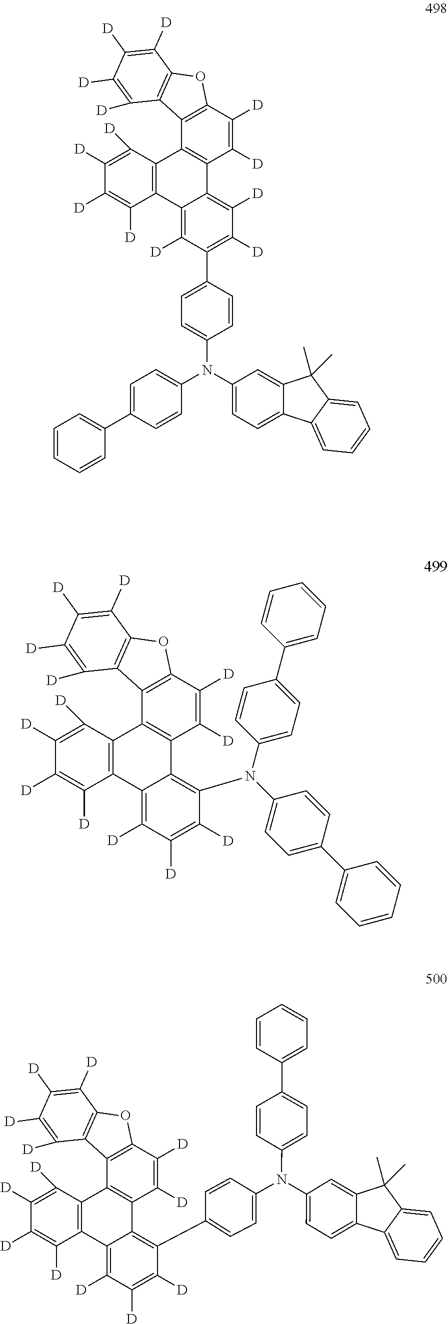

In addition, by introducing various substituents into the structure of Formula 1 above, compounds having intrinsic properties of the introduced substituents can be synthesized. For example, by introducing into the core structure a substituent mainly used in a material for a hole injection layer, a material for a hole transport layer, a material for an electron blocking layer, a material for a light emitting layer, a material for an electron transport layer, a material for a hole blocking layer and a material for a charge generating layer used in manufacturing an organic light emitting device, substances that satisfy the requirements of each organic layer can be synthesized.

Also, by introducing various substituents into the structure of Formula 1, it is possible to finely control the energy bandgap, and on the other hand, to improve the properties at the interface between organic substances, and to diversify the use of the substances.

On the other hand, the compound represented by Formula 1 has a high glass transition temperature (Tg) and thus an excellent thermal stability. This increase in thermal stability is an important factor in providing operational stability to the device.

Also, in one embodiment of the present invention, the present invention provides an organic light emitting device comprising a first electrode; a second electrode provided to face the first electrode; and one or more organic layers provided between the first electrode and the second electrode, wherein at least one of the one or more organic layers comprises a heterocyclic compound represented by Formula 1 above.

In one embodiment of the present invention, the first electrode may be a positive electrode, and the second electrode may be a negative electrode.

In another embodiment, the first electrode may be a negative electrode, and the second electrode may be a positive electrode.

In one embodiment of the present invention, the organic layer may comprise at least one selected from the group consisting of an electron injection layer, an electron transport layer, a hole blocking layer, a light emitting layer, an electron blocking layer, an electron transport layer, and an electron injection layer, and the least one layer selected from the group consisting of the electron injection layer, the electron transport layer, the hole blocking layer, the light emitting layer, the electron blocking layer, the electron transport layer and the electron injection layer may comprise the heterocyclic compound represented by Formula 1 above.

In other embodiment of the present invention, the organic layer may comprise a hole transport layer, and the hole transport layer may comprise the heterocyclic compound represented by Formula 1 above.

In other embodiment of the present invention, the organic layer may comprise an electron blocking layer, and the electron blocking layer may comprise the heterocyclic compound represented by Formula 1 above.

In one embodiment of the present invention, the organic light emitting device may be a blue organic light emitting device, and the heterocyclic compound represented by Formula 1 above may be used as a material of the blue organic light emitting device.

In one embodiment of the present invention, the organic light emitting device may be a green organic light emitting device, and the heterocyclic compound represented by Formula 1 may be used as a material of the green organic light emitting device.

In one embodiment of the present invention, the organic light emitting device may be a red organic light emitting device, and the heterocyclic compound represented by Formula 1 may be used as a material of the red organic light emitting device.

In one embodiment of the present invention, the organic light emitting device may be a blue organic light emitting device, and the heterocyclic compound represented by Formula 1 may be used as a material for a light emitting layer of the blue organic light emitting device.

In one embodiment of the present invention, the organic light emitting device may be a green organic light emitting device, and the heterocyclic compound represented by Formula 1 may be used as a material for a light emitting layer of the green organic light emitting device.

In one embodiment of the present invention, the organic light emitting device may be a red organic light emitting device, and the heterocyclic compound represented by Formula 1 may be used as a material for a light emitting layer of the red organic light emitting device.

Specific details of the heterocyclic compound represented by Formula 1 are the same as described above.

In the organic light emitting device according to an embodiment of the present invention, the organic layer may include an electron injection layer or an electron transport layer, and the electron injection layer or the electron transport layer may include the heterocyclic compound represented by Formula 1.

In the organic light emitting device according to another embodiment, the organic layer may comprise an electron blocking layer or a hole blocking layer, and the electron blocking layer or the hole blocking layer may comprise the heterocyclic compound represented by Formula 1.

In the organic light emitting device according to another embodiment, the organic layer may comprise an electron transport layer, a light emitting layer, or a hole blocking layer, and the electron transport layer, the light emitting layer, or the hole blocking layer may comprise the heterocyclic compound represented by Formula 1.

In the organic light emitting device according to another embodiment, the organic layer may comprise a hole transport layer or an electron blocking layer, and the hole transport layer or the electron blocking layer may comprise the heterocyclic compound represented by Formula 1.

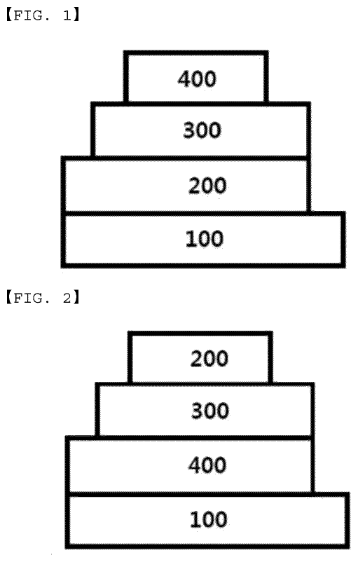

FIGS. 1 to 3 illustrate the stacking order of the electrode and the organic layer of the organic light emitting device according to an embodiment of the present invention. However, it is not intended that the scope of the present application be limited by these drawings, and the structure of the organic light emitting device known in the art may also be applied to the present application.

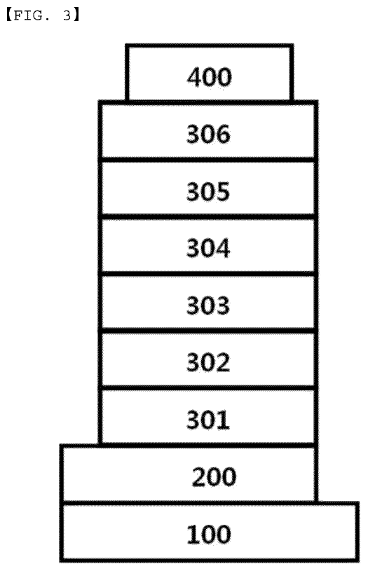

According to FIG. 1, an organic light emitting device formed by sequentially stacking a positive electrode 200, an organic layer 300, and a negative electrode 400 on a substrate 100 is shown. However, it is not limited to such a structure, and an organic light emitting device formed by sequentially stacking a negative electrode, an organic layer, and a positive electrode on a substrate may be implemented, as shown in FIG. 2.

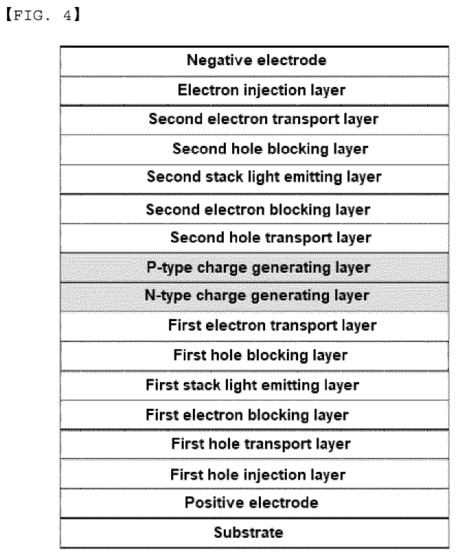

FIG. 3 illustrates a case in which the organic layer is composed of multiple layers. The organic light emitting device according to FIG. 3 comprises a hole injection layer 301, a hole transport layer 302, a light emitting layer 303, a hole blocking layer 304, an electron transport layer 305, and an electron injection layer 306. However, the scope of the present application is not limited by the stacked structure as described above, and if necessary, the remaining layers except for the light emitting layer may be omitted, and other necessary functional layers may be further added.

According to an embodiment of the present invention, the organic light emitting device may have a tandem structure in which two or more independent devices are connected in series. In one embodiment, the tandem structure may be in a form in which each organic light emitting device is bonded through a charge generating layer. Since the device with the tandem structure can operate at a lower current than each unit device based on the same luminance, there is an advantage that the lifetime characteristics of the device are greatly improved.

According to one embodiment of the present invention, the organic layer comprises the first stack comprising one or more light emitting layers; the second stack comprising one or more light emitting layers; and one or more charge generating layers provided between the first stack and the second stack.

According to another embodiment of the present invention, the organic layer comprises the first stack comprising one or more light emitting layers; the second stack comprising one or more light emitting layers; and the third stack comprising one or more light emitting layers, and comprises one or more charge generating layers between the first stack and the second stack and between the second stack and the third stack, respectively.

The term charge generating layer may mean a layer that generates holes and electrons when a voltage is applied thereto. The charge generating layer may be an N-type charge generating layer or a P-type charge generating layer. In the present invention, the term N-type charge generating layer means a charge generating layer located closer to the positive electrode than the P-type charge generating layer, and the term P-type charge generating layer means a charge generating layer located closer to the negative electrode than the N-type charge generating layer.

The N-type charge generating layer and the P-type charge generating layer may be provided in contact, and in this case, an N+P junction is formed. By the N+P junction, holes are easily formed in the P-type charge generating layer and electrons are easily formed in the N-type charge generating layer. The electrons are transported in the direction of the positive electrode through the LUMO level of the N-type charge generating layer, and holes are transported in the direction of the negative electrode through the HOMO level of the P-type charge generating layer.

The first stack, the second stack and the third stack each independently comprise one or more light emitting layers, and may additionally comprise a hole injection layer, a hole transport layer, an electron blocking layer, an electron injection layer, an electron transport layer, a hole blocking layer, and one or more layers among a layer that transports and injects holes at the same time (hole injection and transport layer), and a layer that simultaneously transports and injects electrons (electron injection and transport layer).

An organic light emitting device comprising the first stack and the second stack is illustrated in FIG. 4. However, it is not intended that the scope of the present invention be limited by these drawings, and the structure of an organic light emitting device known in the art may also be applied to the present invention

The first electron blocking layer, the first hole blocking layer, and the second hole blocking layer illustrated in FIG. 4 may be omitted in some cases.

In addition, one embodiment of the present invention provides a composition for organic layer of an organic light emitting device comprising the heterocyclic compound represented by Formula 1 above.

Specific details of the heterocyclic compound represented by Formula 1 are the same as described above.

The composition for organic layer of the organic light emitting device may be used when forming an organic material of the organic light emitting device, and in particular, it may be more preferably used when forming a hole transport layer or an electron blocking layer.

The organic light emitting device of the present invention may be manufactured by a conventional method and material for manufacturing an organic light emitting device, except that one or more organic layers are formed using the aforementioned heterocyclic compound.

The heterocyclic compound may be formed as an organic layer by a solution coating method as well as a vacuum deposition method when manufacturing an organic light emitting device. In this case, the solution coating method means, but is not limited to, spin coating, dip coating, inkjet printing, screen printing, spraying, roll coating, and the like.

The organic layer of the organic light emitting device of the present invention may have a single-layer structure, and may also have a multi-layer structure formed by stacking two or more organic layers. For example, the organic light emitting device of the present invention may have a structure comprising an electron injection layer, an electron transport layer, a hole blocking layer, a light emitting layer, an electron blocking layer, an electron transport layer, an electron injection layer and the like as an organic layer. However, the structure of the organic light emitting device is not limited thereto and may include a smaller number of organic layers.

In one embodiment of the present invention, the present invention provides a method of manufacturing an organic light emitting device comprising the steps of preparing a substrate; forming a first electrode on the substrate; forming one or more organic layers on the first electrode; and forming a second electrode on the one or more organic layers, wherein the step of forming the one or more organic layers comprises a step of forming the one or more organic layers using the composition for organic layer according to an embodiment of the present invention.

In the organic light emitting device according to an embodiment of the present invention, the materials other than the heterocyclic compound represented by Formula 1 are exemplified below, but these are for illustrative purposes only and are not intended to limit the scope of the present application, and may be replaced by materials known in the art.

As the positive electrode material, materials having a relatively large work function can be used, and a transparent conductive oxide, metal, a conductive polymer or the like may be used. Specific examples of the positive electrode material may be, but is not limited to, metals such as vanadium, chromium, copper, zinc, and gold or alloys thereof; metal oxides such as zinc oxide, indium oxide, indium tin oxide (ITO), and indium zinc oxide (IZO); a combination of metal and oxide such as ZnO:Al or SnO₂:Sb; conductive polymers such as poly(3-methylthiophene), poly[3,4-(ethylene-1,2-dioxy)thiophene](PEDOT), polypyrrole and polyaniline; and the like.

As the negative electrode material, materials having a relatively low work function may be used, and metal, metal oxide, a conductive polymer or the like may be used. Specific examples of the negative electrode material may be, but is not limited to, metals such as magnesium, calcium, sodium, potassium, titanium, indium, yttrium, lithium, gadolinium, aluminum, silver, tin and lead or alloys thereof; multilayer-structured materials such as LiF/Al or LiO₂/Al; and the like.

As the hole injection layer material, a known material for the hole injection layer may be used, and for example, phthalocyanine compounds such as copper phthalocyanine disclosed in U.S. Pat. No. 4,356,429, or starburst-type amine derivatives described in [Advanced Material, 6, p. 677 (1994)], such as tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid which is a soluble conductive polymer, or poly(3,4-ethylenedioxythiophene)/poly(4-styrenesulfonate), polyaniline/camphor sulfonic acid, polyaniline/poly(4-styrene-sulfonate), or the like may be used.

As a material for the hole transport layer, a pyrazoline derivative, an arylamine-based derivative, a stilbene derivative, a triphenyldiamine derivative, or the like may be used, and a low-molecular or high-molecular material may be used.

As a material for the electron transport layer, metal complexes of oxadiazole derivatives, anthraquinodimethane and derivatives thereof, benzoquinone and derivatives thereof, naphthoquinone and its derivatives, anthraquinone and its derivatives, tetracyanoanthraquinodimethane and its derivatives, fluorenone derivatives, diphenyldicyanoethylene and its derivatives, diphenoquinone derivatives, 8-hydroxyquinoline and its derivatives, and the like may be used, and high-molecular materials as well as low-molecular materials may be also used.

As the electron injection layer material, for example, LiF is typically used in the art, but the present application is not limited thereto.

As a material for the light emitting layer, a red, green, or blue light emitting material may be used, and if necessary, two or more light emitting materials may be mixed and used. In this case, 2 or more luminescent materials may be used by deposition from separate sources, or may be pre-mixed and deposited from one source. In addition, as a material for the light emitting layer, a fluorescent material may be used, and a phosphorescent material may also be used. As a material for the light emitting layer, a material that emits light by combining holes and electrons respectively injected from the positive electrode and the negative electrode alone may be used, and materials in which the host material and the dopant material together participate in light emission may be also used.

If the hosts of the material for the light emitting layer are mixed and used, hosts of the same type may be mixed and used, or hosts of different types may be mixed and used. For example, any two or more types of n-type host material or p-type host material may be selected and used as a host material for the light emitting layer.

The organic light emitting device according to an embodiment of the present invention may be a top emission type, a bottom emission type, or a double side emission type depending on the material used.

The heterocyclic compound according to an embodiment of the present invention may also act in organic electronic devices comprising an organic solar cell, an organic photoreceptor, an organic transistor, and the like through the principle similar to that applied to the organic light emitting device.

Hereinafter, preferred examples are presented to help the understanding of the present invention. However, the following examples are provided only to make the present invention easier to understand, and the present invention is not limited thereto.

### PREPARATION EXAMPLE

### <Preparation Example 1> Preparation of Compound 12

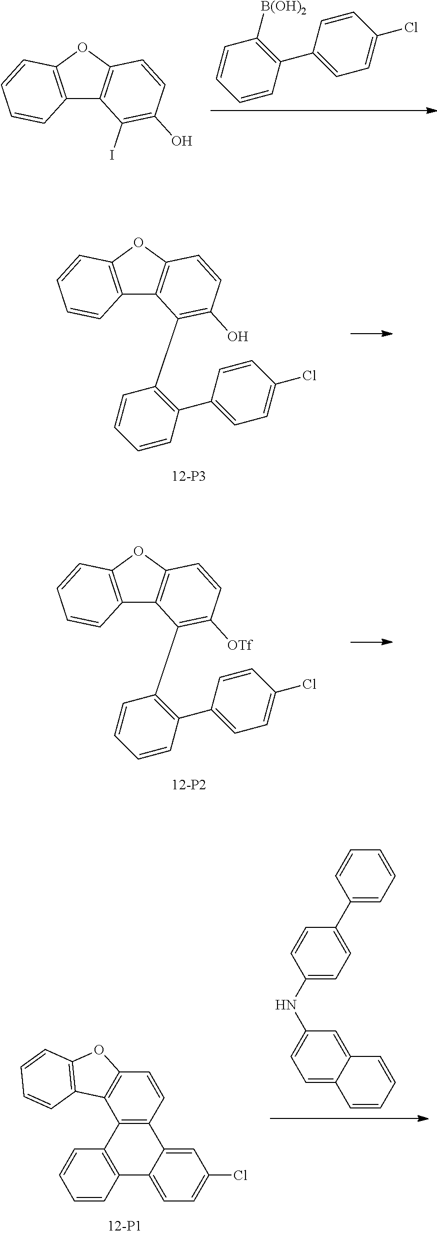 [SMILES: Oc1ccc2oc3ccccc3c2c1I ; OB(O)c1ccccc1-c1ccc(Cl)cc1 ; Oc1ccc2oc3ccccc3c2c1-c1ccccc1-c1ccc(Cl)cc1 ; O=S(=O)(Oc1ccc2oc3ccccc3c2c1-c1ccccc1-c1ccc(Cl)cc1)C(F)(F)F ; Clc1ccc2c3ccccc3c3c(ccc4oc5ccccc5c43)c2c1 ; c1ccc(-c2ccc(Nc3ccc4ccccc4c3)cc2)cc1]

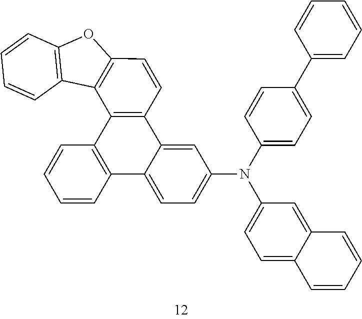 [SMILES: c1ccc(-c2ccc(N(c3ccc4ccccc4c3)c3ccc4c5ccccc5c5c(ccc6oc7ccccc7c65)c4c3)cc2)cc1]

1) Preparation of Compound 12-P3

100 g of 1-iododibenzo[b,d]furan-2-ol (322.58 mmol) and 75 g of (4′-chloro-[1,1′-biphenyl]-2-yl)boronic acid (322.58 mmol) were dissolved in 1500 mL of 1,4-dioxane and 200 mL of distilled water, and then 18.63 g of tetrakis(triphenylhosphine)palladium(0) (Pd(PPh₃)₄) (16.13 mmol) and 133.74 g of K₂CO₃ (967.74 mmol) were added, and the mixture was stirred under reflux for 12 hours. After completion of the reaction, ethyl acetate was added and dissolved in the reaction solution, and then extracted with distilled water, and the organic layer was dried over anhydrous MgSO₄, and then the solvent was removed using a rotary evaporator. Thereafter, it was purified by column chromatography using dichloromethane and hexane as developing solvents to obtain 104 g of compound 12-P3 (yield 87%).

2) Preparation of Compound 12-P2

104 g of the compound 12-P3 (280.64 mmol) was dissolved in 2000 mL of dichloromethane and 47 mL of triethylamine, and then 56.5 mL of trifluoromethanesulfonic anhydride (triflic anhydride) (336.54 mmol) was added, and stirred at room temperature for 4 hours. After completion of the reaction, distilled water was added to the reaction solution for extraction, and the organic layer was dried over anhydrous MgSO₄, and then the solvent was removed using a rotary evaporator. It was purified by column chromatography using dichloromethane and hexane as developing solvents to obtain 104 g of compound 12-P2 (yield: 85%).

3) Preparation of Compound 12-P1

104 g of the compound 12-P2 (206.80 mmol) was dissolved in 1000 ml of 1-methyl-2-pyrrolidinone, and then 2.32 g of palladium (II) acetate (Pd(OAc) 2) (10.34 mmol), 5.42 g of triphenylphosphine (PPh₃) (20.68 mmol), and 134.76 g of Cs₂CO₃ (413.60 mmol) were added and stirred under reflux for 12 hours. After completion of the reaction, the mixture was extracted with dichloromethane and distilled water, the organic layer was dried over anhydrous MgSO₄, and then the solvent was removed by a rotary evaporator, and thereafter, it was purified by column chromatography using dichloromethane and hexane as developing solvents to obtain 53 g of compound 12-P1 (yield 72%).

4) Preparation of Compound 12

10 g of the compound 12-P1 (30.41 mmol) and 7.83 g of N-([1,1′-biphenyl]-4-yl)naphthalen-2-amine (31.94 mmol) were dissolved in 100 ml of xylene, and then 1.39 g of tris(dibenzylideneacetone)dipalladium (Pd₂(dba)₃) (1.52 mmol), 1.42 ml of tri-tert-butylphosphine(P(t-Bu)₃) (3.04 mmol), and 7.31 g of t-BuONa (76.04 mmol) were added, and the mixture was stirred under reflux for 3 hours. After completion of the reaction, methylene chloride (MC) was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 13 g of compound 12 (yield 79%).

The target compounds shown in Table 1 below were synthesized in the same manner as in Preparation Example 1, except that in Preparation Example 1, Compound A below was used instead of (4′-chloro-[1,1′-biphenyl]-2-yl)boronic acid, and Compound B below was used instead of N-([1,1′-biphenyl]-4-yl)naphthalen-2-amine.

TABLE 1

| Com- | | | | |
|---|---|---|---|---|
| pound | | | | |
| no. | Compound A | Compound B | Target compound | Yield |
| 20 | 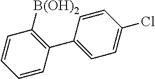 [SMILES: OB(O)c1ccccc1-c1ccc(Cl)cc1] | 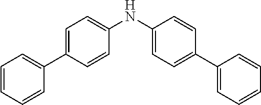 [SMILES: c1ccc(-c2ccc(Nc3ccc(-c4ccccc4)cc3)cc2)cc1] | 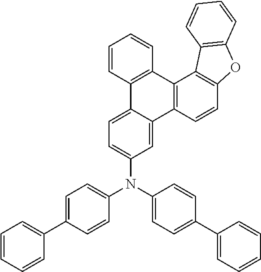 [SMILES: c1ccc(-c2ccc(N(c3ccc(-c4ccccc4)cc3)c3ccc4c5ccccc5c5c(ccc6oc7ccccc7c65)c4c3)cc2)cc1] | 66% |
| 25 | 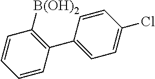 [SMILES: OB(O)c1ccccc1-c1ccc(Cl)cc1] | 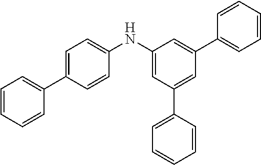 [SMILES: c1ccc(-c2ccc(Nc3cc(-c4ccccc4)cc(-c4ccccc4)c3)cc2)cc1] | 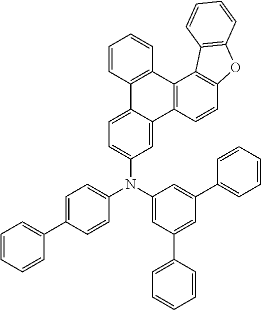 [SMILES: c1ccc(-c2ccc(N(c3cc(-c4ccccc4)cc(-c4ccccc4)c3)c3ccc4c5ccccc5c5c(ccc6oc7ccccc7c65)c4c3)cc2)cc1] | 71% |
| 29 | 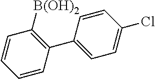 [SMILES: OB(O)c1ccccc1-c1ccc(Cl)cc1] | 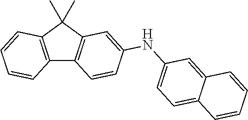 [SMILES: CC1(C)c2ccccc2-c2ccc(Nc3ccc4ccccc4c3)cc21] | 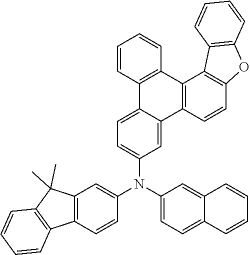 [SMILES: CC1(C)c2ccccc2-c2ccc(N(c3ccc4ccccc4c3)c3ccc4c5ccccc5c5c(ccc6oc7ccccc7c65)c4c3)cc21] | 68% |
| 31 | 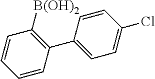 [SMILES: OB(O)c1ccccc1-c1ccc(Cl)cc1] | 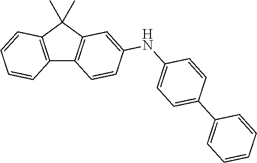 [SMILES: CC1(C)c2ccccc2-c2ccc(Nc3ccc(-c4ccccc4)cc3)cc21] | 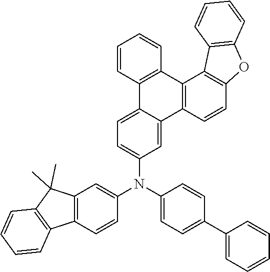 [SMILES: CC1(C)c2ccccc2-c2ccc(N(c3ccc(-c4ccccc4)cc3)c3ccc4c5ccccc5c5c(ccc6oc7ccccc7c65)c4c3)cc21] | 70% |
| 35 | 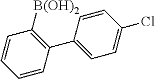 [SMILES: OB(O)c1ccccc1-c1ccc(Cl)cc1] | 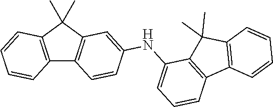 [SMILES: CC1(C)c2ccccc2-c2ccc(Nc3cccc4c3C(C)(C)c3ccccc3-4)cc21] | 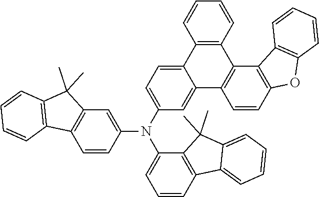 [SMILES: CC1(C)c2ccccc2-c2ccc(N(c3ccc4c5ccccc5c5c(ccc6oc7ccccc7c65)c4c3)c3cccc4c3C(C)(C)c3ccccc3-4)cc21] | 69% |
| 38 | 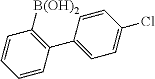 [SMILES: OB(O)c1ccccc1-c1ccc(Cl)cc1] | 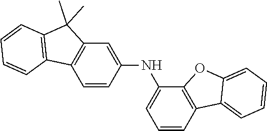 [SMILES: CC1(C)c2ccccc2-c2ccc(Nc3cccc4c3oc3ccccc34)cc21] | 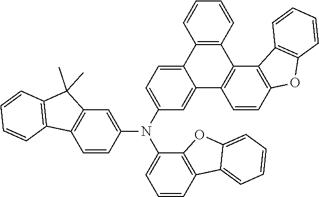 [SMILES: CC1(C)c2ccccc2-c2ccc(N(c3ccc4c5ccccc5c5c(ccc6oc7ccccc7c65)c4c3)c3cccc4c3oc3ccccc34)cc21] | 72% |
| 43 | 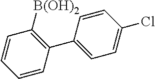 [SMILES: OB(O)c1ccccc1-c1ccc(Cl)cc1] | 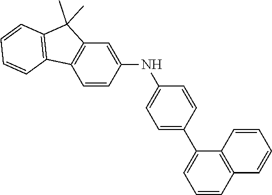 [SMILES: CC1(C)c2ccccc2-c2ccc(Nc3ccc(-c4cccc5ccccc45)cc3)cc21] | 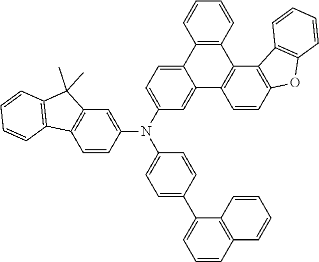 [SMILES: CC1(C)c2ccccc2-c2ccc(N(c3ccc(-c4cccc5ccccc45)cc3)c3ccc4c5ccccc5c5c(ccc6oc7ccccc7c65)c4c3)cc21] | 73% |
| 47 | 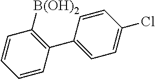 [SMILES: OB(O)c1ccccc1-c1ccc(Cl)cc1] | 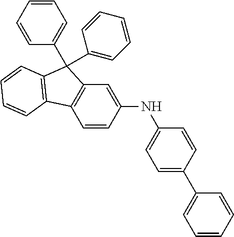 [SMILES: c1ccc(-c2ccc(Nc3ccc4c(c3)C(c3ccccc3)(c3ccccc3)c3ccccc3-4)cc2)cc1] | 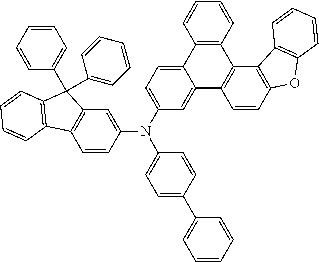 [SMILES: c1ccc(-c2ccc(N(c3ccc4c(c3)C(c3ccccc3)(c3ccccc3)c3ccccc3-4)c3ccc4c5ccccc5c5c(ccc6oc7ccccc7c65)c4c3)cc2)cc1] | 68% |
| 55 | 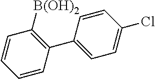 [SMILES: OB(O)c1ccccc1-c1ccc(Cl)cc1] | 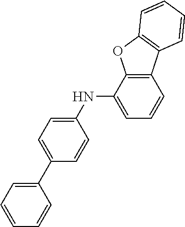 [SMILES: c1ccc(-c2ccc(Nc3cccc4c3oc3ccccc34)cc2)cc1] | 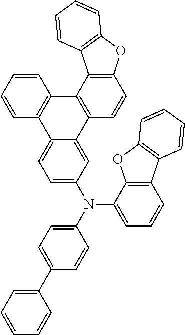 [SMILES: c1ccc(-c2ccc(N(c3ccc4c5ccccc5c5c(ccc6oc7ccccc7c65)c4c3)c3cccc4c3oc3ccccc34)cc2)cc1] | 77% |
| 56 | 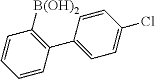 [SMILES: OB(O)c1ccccc1-c1ccc(Cl)cc1] | 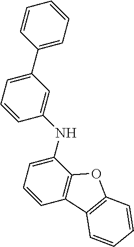 [SMILES: c1ccc(-c2cccc(Nc3cccc4c3oc3ccccc34)c2)cc1] | 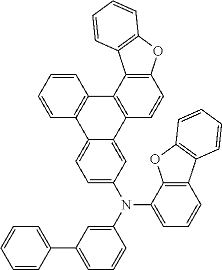 [SMILES: c1ccc(-c2cccc(N(c3ccc4c5ccccc5c5c(ccc6oc7ccccc7c65)c4c3)c3cccc4c3oc3ccccc34)c2)cc1] | 72% |
| 242 | 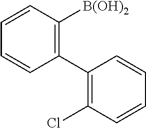 [SMILES: OB(O)c1ccccc1-c1ccccc1Cl] | 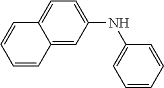 [SMILES: c1ccc(Nc2ccc3ccccc3c2)cc1] | 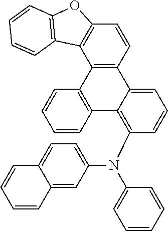 [SMILES: c1ccc(N(c2ccc3ccccc3c2)c2cccc3c4ccc5oc6ccccc6c5c4c4ccccc4c23)cc1] | 68% |
| 260 | 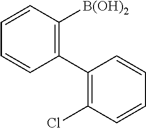 [SMILES: OB(O)c1ccccc1-c1ccccc1Cl] | 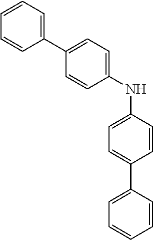 [SMILES: c1ccc(-c2ccc(Nc3ccc(-c4ccccc4)cc3)cc2)cc1] | 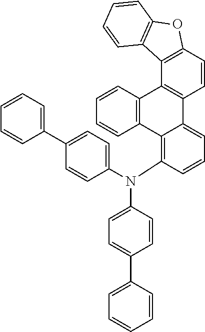 [SMILES: c1ccc(-c2ccc(N(c3ccc(-c4ccccc4)cc3)c3cccc4c5ccc6oc7ccccc7c6c5c5ccccc5c34)cc2)cc1] | 73% |
| 265 | 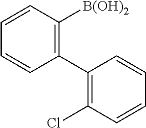 [SMILES: OB(O)c1ccccc1-c1ccccc1Cl] | 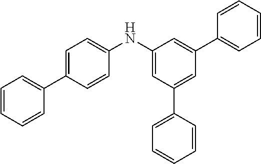 [SMILES: c1ccc(-c2ccc(Nc3cc(-c4ccccc4)cc(-c4ccccc4)c3)cc2)cc1] | 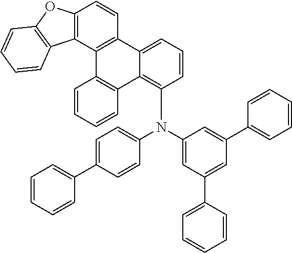 [SMILES: c1ccc(-c2ccc(N(c3cc(-c4ccccc4)cc(-c4ccccc4)c3)c3cccc4c5ccc6oc7ccccc7c6c5c5ccccc5c34)cc2)cc1] | 70% |
| 268 | 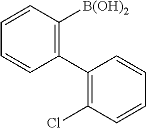 [SMILES: OB(O)c1ccccc1-c1ccccc1Cl] | 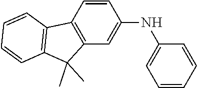 [SMILES: CC1(C)c2ccccc2-c2ccc(Nc3ccccc3)cc21] | 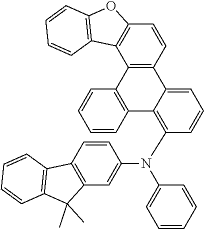 [SMILES: CC1(C)c2ccccc2-c2ccc(N(c3ccccc3)c3cccc4c5ccc6oc7ccccc7c6c5c5ccccc5c34)cc21] | 75% |
| 269 | 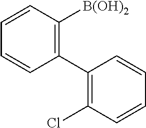 [SMILES: OB(O)c1ccccc1-c1ccccc1Cl] | 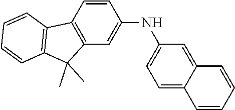 [SMILES: CC1(C)c2ccccc2-c2ccc(Nc3ccc4ccccc4c3)cc21] | 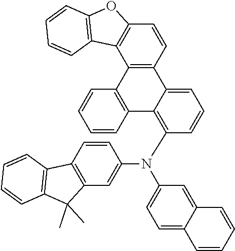 [SMILES: CC1(C)c2ccccc2-c2ccc(N(c3ccc4ccccc4c3)c3cccc4c5ccc6oc7ccccc7c6c5c5ccccc5c34)cc21] | 73% |
| 271 | 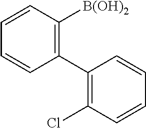 [SMILES: OB(O)c1ccccc1-c1ccccc1Cl] | 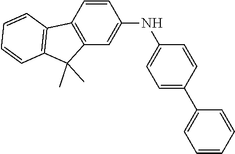 [SMILES: CC1(C)c2ccccc2-c2ccc(Nc3ccc(-c4ccccc4)cc3)cc21] | 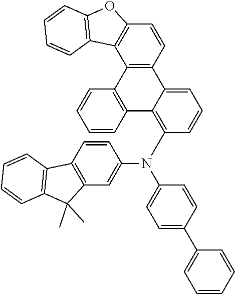 [SMILES: CC1(C)c2ccccc2-c2ccc(N(c3ccc(-c4ccccc4)cc3)c3cccc4c5ccc6oc7ccccc7c6c5c5ccccc5c34)cc21] | 71% |
| 278 | 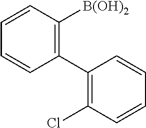 [SMILES: OB(O)c1ccccc1-c1ccccc1Cl] | 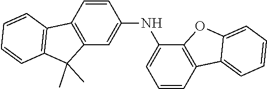 [SMILES: CC1(C)c2ccccc2-c2ccc(Nc3cccc4c3oc3ccccc34)cc21] | 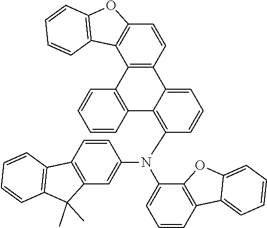 [SMILES: CC1(C)c2ccccc2-c2ccc(N(c3cccc4c3oc3ccccc34)c3cccc4c5ccc6oc7ccccc7c6c5c5ccccc5c34)cc21] | 69% |
| 295 | 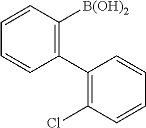 [SMILES: OB(O)c1ccccc1-c1ccccc1Cl] | 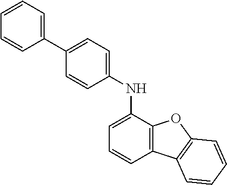 [SMILES: c1ccc(-c2ccc(Nc3cccc4c3oc3ccccc34)cc2)cc1] | 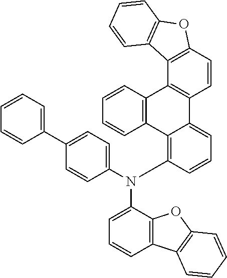 [SMILES: c1ccc(-c2ccc(N(c3cccc4c3oc3ccccc34)c3cccc4c5ccc6oc7ccccc7c6c5c5ccccc5c34)cc2)cc1] | 70% |
| 298 | 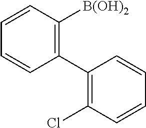 [SMILES: OB(O)c1ccccc1-c1ccccc1Cl] | 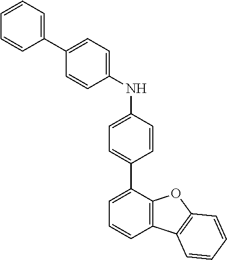 [SMILES: c1ccc(-c2ccc(Nc3ccc(-c4cccc5c4oc4ccccc45)cc3)cc2)cc1] | 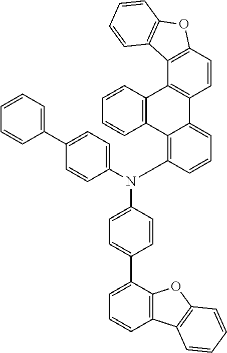 [SMILES: c1ccc(-c2ccc(N(c3ccc(-c4cccc5c4oc4ccccc45)cc3)c3cccc4c5ccc6oc7ccccc7c6c5c5ccccc5c34)cc2)cc1] | 72% |

### <Preparation Example 2> Preparation of Compound 72

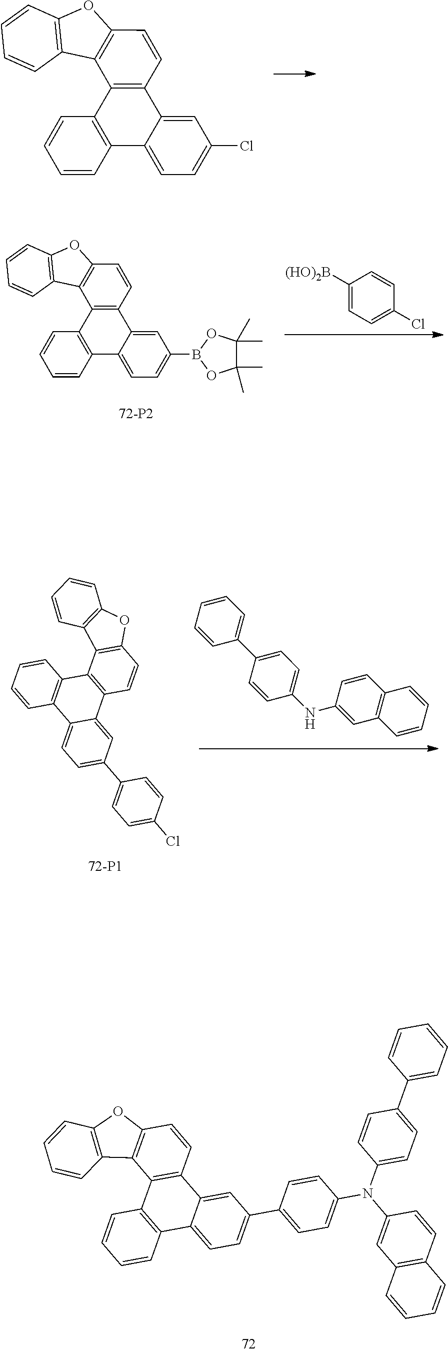 [SMILES: Clc1ccc2c3ccccc3c3c(ccc4oc5ccccc5c43)c2c1 ; CC1(C)OB(c2ccc3c4ccccc4c4c(ccc5oc6ccccc6c54)c3c2)OC1(C)C ; OB(O)c1ccc(Cl)cc1 ; Clc1ccc(-c2ccc3c4ccccc4c4c(ccc5oc6ccccc6c54)c3c2)cc1 ; c1ccc(-c2ccc(Nc3ccc4ccccc4c3)cc2)cc1 ; c1ccc(-c2ccc(N(c3ccc(-c4ccc5c6ccccc6c6c(ccc7oc8ccccc8c76)c5c4)cc3)c3ccc4ccccc4c3)cc2)cc1]

1) Preparation of Compound 72-P2

20 g of 3-chlorotriphenyleno[2,1-b]benzofuran (56.68 mmol) and 21.6 g of 4,4,4′,4′,5,5,5′,5′-octamethyl-2,2′-bi(1,3,2-dioxaborolane) (85.02 mmol) were dissolved in 300 mL of 1,4-dioxane, and then 3.25 g of bis(dibenzylideneacetone)palladium (Pd(dba) 2) (5.13 mmol), 16.68 g of potassium acetate (170.04 mmol), and 4.89 g of dicyclohexyl (2′,4′,6′-triisopropyl-[1,1′-biphenyl]-2-yl)phosphine (Xphos) (10.26 mmol) were added, and the mixture was stirred under reflux for 12 hours. After completion of the reaction, ethyl acetate was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 20 g of compound 72-P2 (yield 75%).

2) Preparation of Compound 72-P1

20 g of the compound 72-P2 (45.01 mmol) and 7.03 g of (4-chlorophenyl)boronic acid (45.01 mmol) were dissolved in 200 mL of 1,4-dioxane and 40 mL of distilled water, and then 2.60 g of tetrakis(triphenylhosphine)palladium(0) (Pd(PPh₃)₄) (2.25 mmol) and 18.66 g of K₂CO₃ (135.03 mmol) were added, and the mixture was stirred under reflux for 12 hours. After completion of the reaction, ethyl acetate was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 16.79 g of compound 72-P1 (yield 87%).

3) Preparation of Compound 72

10 g of the compound 72-P1 (23.31 mmol) and 6.88 g of N-([1,1′-biphenyl]-4-yl)naphthalen-2-amine (23.31 mmol) were dissolved in 100 ml of xylene, and then 1.06 g of tris(dibenzylideneacetone)dipalladium(0) (Pd₂(dba₃)) (1.16 mmol), 1.26 mL of tri-tert-butyl phosphine(P(t-Bu) 3) (2.32 mmol) and 6.72 g of t-BuONa (69.93 mmol) were added, and the mixture was stirred under reflux for 3 hours. After completion of the reaction, methylchloride was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 12 g of compound 72 (yield 75%).

The target compounds shown in Table 2 below were synthesized in the same manner as in Preparation Example 2, except that in Preparation Example 2, Compound A below was used instead of (4-chlorophenyl) boronic acid, and Compound B below was used instead of N-([1,1′-biphenyl]-4-yl)naphthalen-2-amine.

TABLE 2

| Com- | | | | |
|---|---|---|---|---|
| pound | | | | |
| no. | Compound A | Compound B | Target compound | Yield |
| 80 | 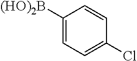 | 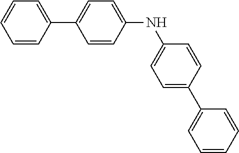 [SMILES: c1ccc(-c2ccc(Nc3ccc(-c4ccccc4)cc3)cc2)cc1] | 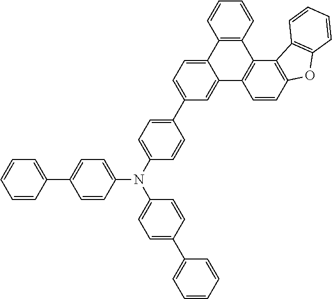 [SMILES: c1ccc(-c2ccc(N(c3ccc(-c4ccccc4)cc3)c3ccc(-c4ccc5c6ccccc6c6c(ccc7oc8ccccc8c76)c5c4)cc3)cc2)cc1] | 66% |
| 91 | 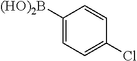 | 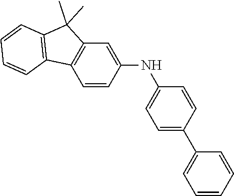 [SMILES: CC1(C)c2ccccc2-c2ccc(Nc3ccc(-c4ccccc4)cc3)cc21] | 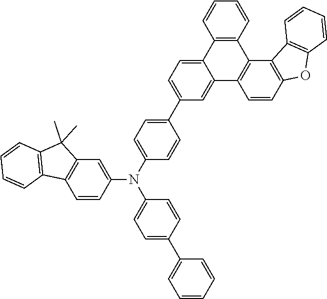 [SMILES: CC1(C)c2ccccc2-c2ccc(N(c3ccc(-c4ccccc4)cc3)c3ccc(-c4ccc5c6ccccc6c6c(ccc7oc8ccccc8c76)c5c4)cc3)cc21] | 75% |
| 98 | 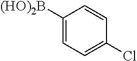 | 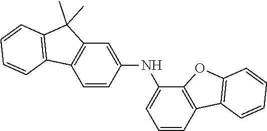 [SMILES: CC1(C)c2ccccc2-c2ccc(Nc3cccc4c3oc3ccccc34)cc21] | 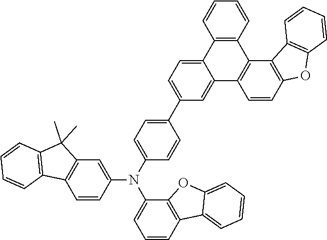 [SMILES: CC1(C)c2ccccc2-c2ccc(N(c3ccc(-c4ccc5c6ccccc6c6c(ccc7oc8ccccc8c76)c5c4)cc3)c3cccc4c3oc3ccccc34)cc21] | 73% |
| 115 | 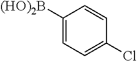 | 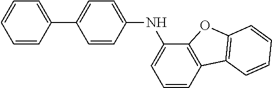 [SMILES: c1ccc(-c2ccc(Nc3cccc4c3oc3ccccc34)cc2)cc1] | 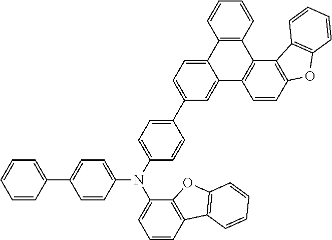 [SMILES: c1ccc(-c2ccc(N(c3ccc(-c4ccc5c6ccccc6c6c(ccc7oc8ccccc8c76)c5c4)cc3)c3cccc4c3oc3ccccc34)cc2)cc1] | 71% |
| 118 | 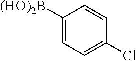 | 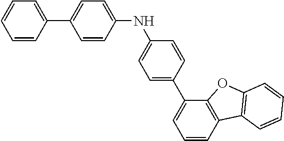 [SMILES: c1ccc(-c2ccc(Nc3ccc(-c4cccc5c4oc4ccccc45)cc3)cc2)cc1] | 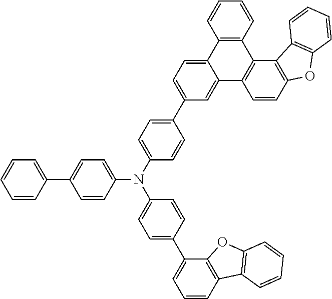 [SMILES: c1ccc(-c2ccc(N(c3ccc(-c4ccc5c6ccccc6c6c(ccc7oc8ccccc8c76)c5c4)cc3)c3ccc(-c4cccc5c4oc4ccccc45)cc3)cc2)cc1] | 69% |
| 122 | 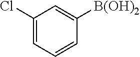 | 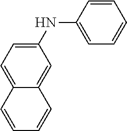 [SMILES: c1ccc(Nc2ccc3ccccc3c2)cc1] | 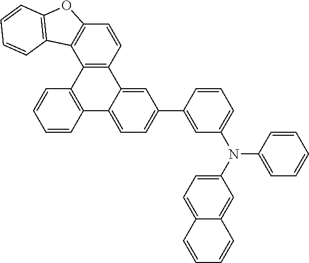 [SMILES: c1ccc(N(c2cccc(-c3ccc4c5ccccc5c5c(ccc6oc7ccccc7c65)c4c3)c2)c2ccc3ccccc3c2)cc1] | 70% |
| 140 | 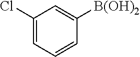 | 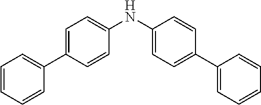 [SMILES: c1ccc(-c2ccc(Nc3ccc(-c4ccccc4)cc3)cc2)cc1] | 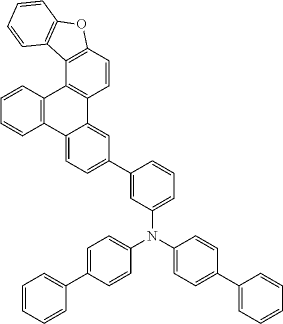 [SMILES: c1ccc(-c2ccc(N(c3ccc(-c4ccccc4)cc3)c3cccc(-c4ccc5c6ccccc6c6c(ccc7oc8ccccc8c76)c5c4)c3)cc2)cc1] | 72% |
| 148 | 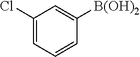 | 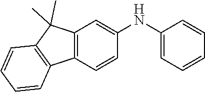 [SMILES: CC1(C)c2ccccc2-c2ccc(Nc3ccccc3)cc21] | 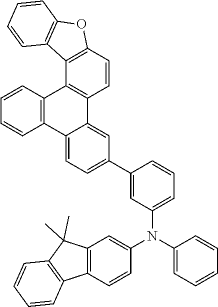 [SMILES: CC1(C)c2ccccc2-c2ccc(N(c3ccccc3)c3cccc(-c4ccc5c6ccccc6c6c(ccc7oc8ccccc8c76)c5c4)c3)cc21] | 74% |
| 151 | 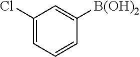 | 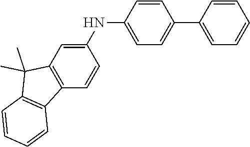 [SMILES: CC1(C)c2ccccc2-c2ccc(Nc3ccc(-c4ccccc4)cc3)cc21] | 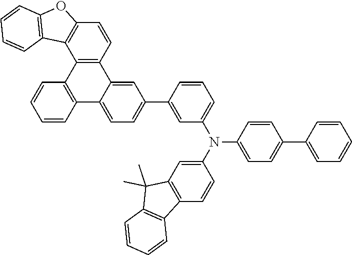 [SMILES: CC1(C)c2ccccc2-c2ccc(N(c3ccc(-c4ccccc4)cc3)c3cccc(-c4ccc5c6ccccc6c6c(ccc7oc8ccccc8c76)c5c4)c3)cc21] | 75% |
| 158 | 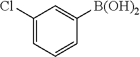 | 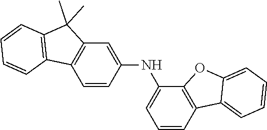 [SMILES: CC1(C)c2ccccc2-c2ccc(Nc3cccc4c3oc3ccccc34)cc21] | 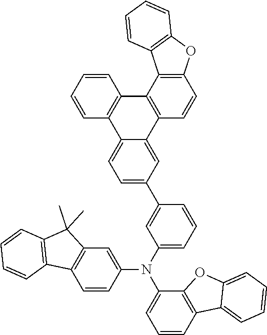 [SMILES: CC1(C)c2ccccc2-c2ccc(N(c3cccc(-c4ccc5c6ccccc6c6c(ccc7oc8ccccc8c76)c5c4)c3)c3cccc4c3oc3ccccc34)cc21] | 73% |
| 175 | 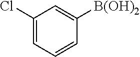 | 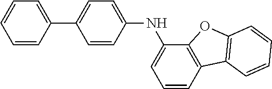 [SMILES: c1ccc(-c2ccc(Nc3cccc4c3oc3ccccc34)cc2)cc1] | 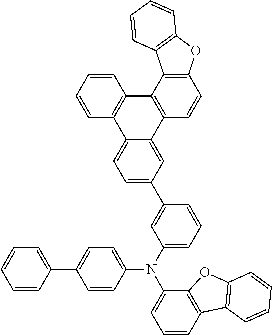 [SMILES: c1ccc(-c2ccc(N(c3cccc(-c4ccc5c6ccccc6c6c(ccc7oc8ccccc8c76)c5c4)c3)c3cccc4c3oc3ccccc34)cc2)cc1] | 71% |
| 178 | 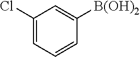 | 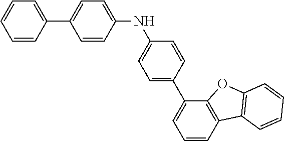 [SMILES: c1ccc(-c2ccc(Nc3ccc(-c4cccc5c4oc4ccccc45)cc3)cc2)cc1] | 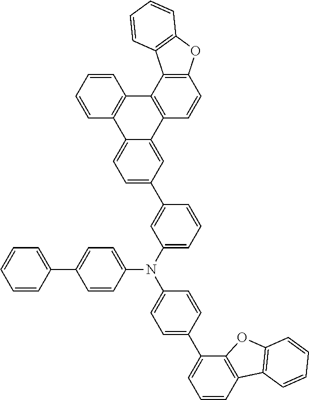 [SMILES: c1ccc(-c2ccc(N(c3ccc(-c4cccc5c4oc4ccccc45)cc3)c3cccc(-c4ccc5c6ccccc6c6c(ccc7oc8ccccc8c76)c5c4)c3)cc2)cc1] | 70% |
| 200 | 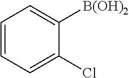 | 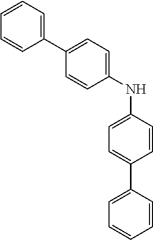 [SMILES: c1ccc(-c2ccc(Nc3ccc(-c4ccccc4)cc3)cc2)cc1] | 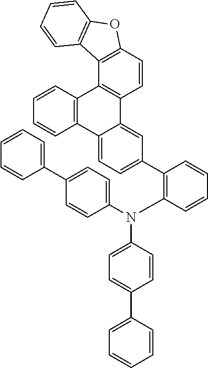 [SMILES: c1ccc(-c2ccc(N(c3ccc(-c4ccccc4)cc3)c3ccccc3-c3ccc4c5ccccc5c5c(ccc6oc7ccccc7c65)c4c3)cc2)cc1] | 68% |
| 211 | 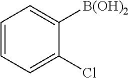 | 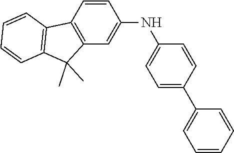 [SMILES: CC1(C)c2ccccc2-c2ccc(Nc3ccc(-c4ccccc4)cc3)cc21] | 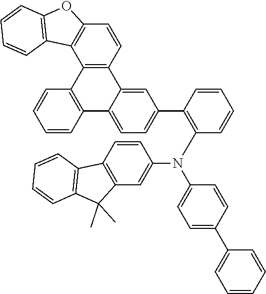 [SMILES: CC1(C)c2ccccc2-c2ccc(N(c3ccc(-c4ccccc4)cc3)c3ccccc3-c3ccc4c5ccccc5c5c(ccc6oc7ccccc7c65)c4c3)cc21] | 70% |
| 218 | 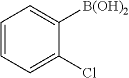 | 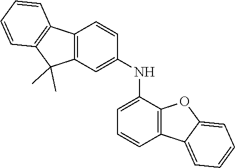 [SMILES: CC1(C)c2ccccc2-c2ccc(Nc3cccc4c3oc3ccccc34)cc21] | 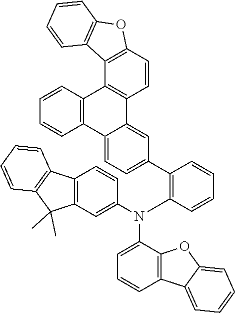 [SMILES: CC1(C)c2ccccc2-c2ccc(N(c3ccccc3-c3ccc4c5ccccc5c5c(ccc6oc7ccccc7c65)c4c3)c3cccc4c3oc3ccccc34)cc21] | 72% |
| 235 | 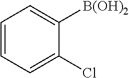 | 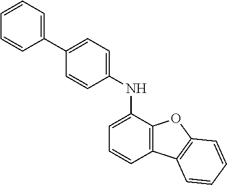 [SMILES: c1ccc(-c2ccc(Nc3cccc4c3oc3ccccc34)cc2)cc1] | 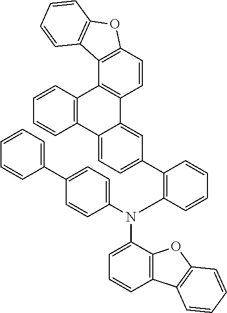 [SMILES: c1ccc(-c2ccc(N(c3ccccc3-c3ccc4c5ccccc5c5c(ccc6oc7ccccc7c65)c4c3)c3cccc4c3oc3ccccc34)cc2)cc1] | 74% |

### [Preparation Example 3] Preparation of Compound 302

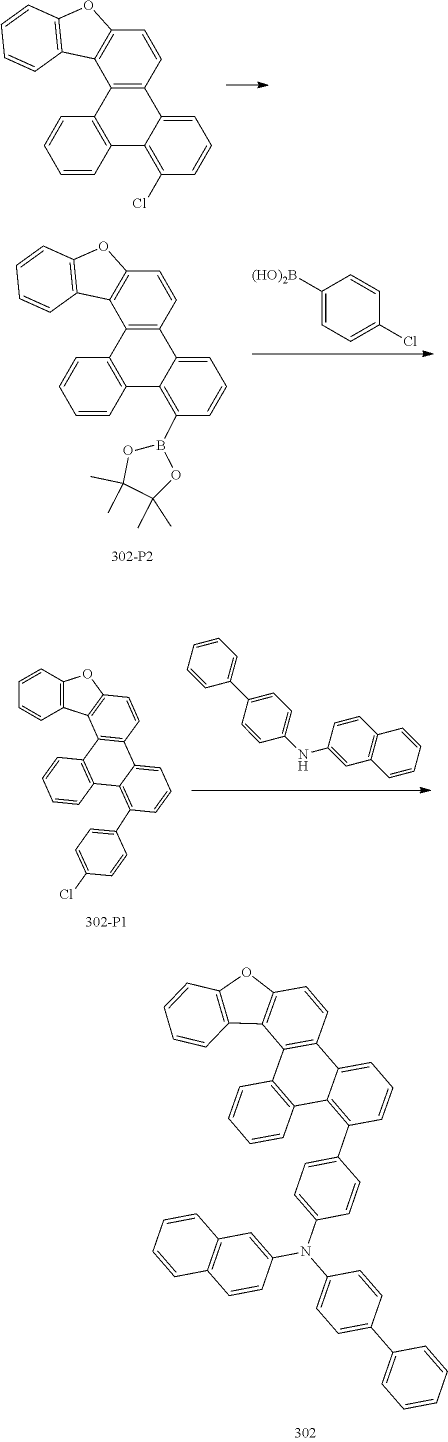 [SMILES: Clc1cccc2c3ccc4oc5ccccc5c4c3c3ccccc3c12 ; CC1(C)OB(c2cccc3c4ccc5oc6ccccc6c5c4c4ccccc4c23)OC1(C)C ; OB(O)c1ccc(Cl)cc1 ; Clc1ccc(-c2cccc3c4ccc5oc6ccccc6c5c4c4ccccc4c23)cc1 ; c1ccc(-c2ccc(Nc3ccc4ccccc4c3)cc2)cc1 ; c1ccc(-c2ccc(N(c3ccc(-c4cccc5c6ccc7oc8ccccc8c7c6c6ccccc6c45)cc3)c3ccc4ccccc4c3)cc2)cc1]

1) Preparation of Compound 302-P2

20 g of 1-chlorotriphenyleno[2,1-b]benzofuran (56.68 mmol) and 21.6 g of 4,4,4′,4′,5,5,5′,5′-octamethyl-2,2′-bi(1,3,2-dioxaborolane) (85.02 mmol) were dissolved in 300 mL of 1,4-dioxane, and then 3.25 g of bis(dibenzylideneacetone)palladium(Pd(dba) 2) (5.13 mmol), 16.68 g of potassium acetate (170.04 mmol), and 4.89 g of dicyclohexyl (2 1,4′,6′-triisopropyl-[1,1′-biphenyl]-2-yl)phosphine (Xphos) (10.26 mmol) were added, and the mixture was stirred under reflux for 12 hours. After completion of the reaction, ethyl acetate was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 20 g of compound 302-P2 (yield 75%).

2) Preparation of Compound 302-P1

20 g of the compound 302-P2 (45.01 mmol) and 7.03 g of (4-chlorophenyl) boronic acid (45.01 mmol) were dissolved in 200 mL of 1,4-dioxane and 40 mL of distilled water, and then 2.60 g of tetrakis(triphenylhosphine)palladium(0) (Pd(PPh₃)₄) (2.25 mmol) and 18.66 g of K₂CO₃ (135.03 mmol) were added, and the mixture was stirred under reflux for 12 hours. After completion of the reaction, ethyl acetate was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 16.79 g of compound 302-P1 (yield 87%).

3) Preparation of Compound 302

10 g of the compound 302-P1 (23.31 mmol) and 6.88 g of N-([1,1′-biphenyl]-4-yl)naphthalen-2-amine (23.31 mmol) were dissolved in 100 ml of xylene, and then 1.06 g of tris(dibenzylideneacetone)dipalladium(0) (Pd₂(dba)₃) (1.16 mmol), 1.26 mL of tri-tert-butyl phosphine(P(t-Bu)₃) (2.32 mmol), and 6.72 g of t-BuONa (69.93 mmol) were added, and the mixture was stirred under reflux for 3 hours. After completion of the reaction, MC was added and dissolved in the reaction solution, and then extracted with distilled water, and the organic layer was dried over anhydrous MgSO₄, and then the solvent was removed by a rotary evaporator. Thereafter, it was purified by column chromatography using dichloromethane and hexane as developing solvents to obtain 12 g of compound 302 (yield 75%).

The target compounds shown in Table 3 below were synthesized in the same manner as in Preparation Example 3, except that in Preparation Example 3, Compound A below was used instead of (4-chlorophenyl)boronic acid, and Compound B below was used instead of N-([1,1′-biphenyl]-4-yl)naphthalen-2-amine.

TABLE 3

| Com- | | | | |
|---|---|---|---|---|
| pound | | | | |
| no. | Compound A | Compound B | Target compound | Yield |
| 320 | 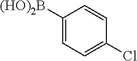 | 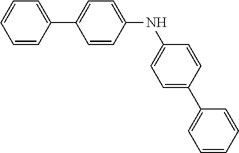 [SMILES: c1ccc(-c2ccc(Nc3ccc(-c4ccccc4)cc3)cc2)cc1] | 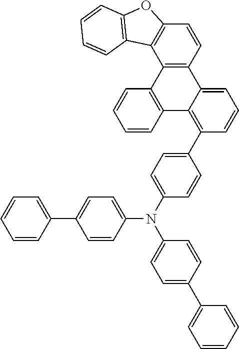 [SMILES: c1ccc(-c2ccc(N(c3ccc(-c4ccccc4)cc3)c3ccc(-c4cccc5c6ccc7oc8ccccc8c7c6c6ccccc6c45)cc3)cc2)cc1] | 66% |
| 331 | 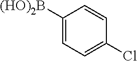 | 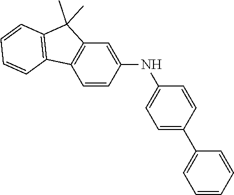 [SMILES: CC1(C)c2ccccc2-c2ccc(Nc3ccc(-c4ccccc4)cc3)cc21] | 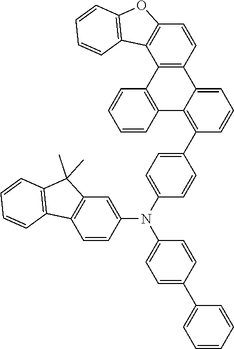 [SMILES: CC1(C)c2ccccc2-c2ccc(N(c3ccc(-c4ccccc4)cc3)c3ccc(-c4cccc5c6ccc7oc8ccccc8c7c6c6ccccc6c45)cc3)cc21] | 73% |
| 338 | 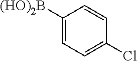 | 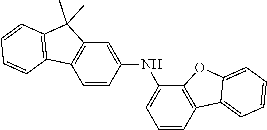 [SMILES: CC1(C)c2ccccc2-c2ccc(Nc3cccc4c3oc3ccccc34)cc21] | 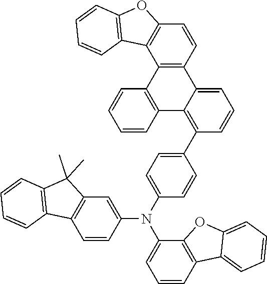 [SMILES: CC1(C)c2ccccc2-c2ccc(N(c3ccc(-c4cccc5c6ccc7oc8ccccc8c7c6c6ccccc6c45)cc3)c3cccc4c3oc3ccccc34)cc21] | 74% |
| 355 | 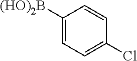 | 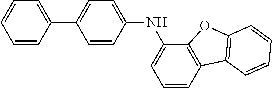 [SMILES: c1ccc(-c2ccc(Nc3cccc4c3oc3ccccc34)cc2)cc1] | 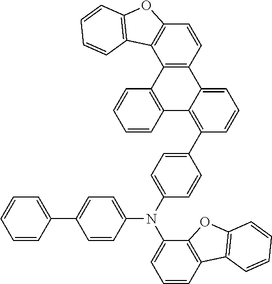 [SMILES: c1ccc(-c2ccc(N(c3ccc(-c4cccc5c6ccc7oc8ccccc8c7c6c6ccccc6c45)cc3)c3cccc4c3oc3ccccc34)cc2)cc1] | 72% |
| 358 | 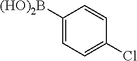 | 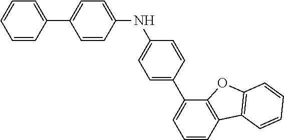 [SMILES: c1ccc(-c2ccc(Nc3ccc(-c4cccc5c4oc4ccccc45)cc3)cc2)cc1] | 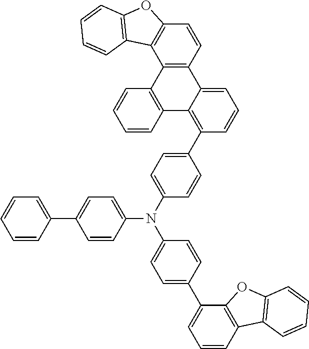 [SMILES: c1ccc(-c2ccc(N(c3ccc(-c4cccc5c4oc4ccccc45)cc3)c3ccc(-c4cccc5c6ccc7oc8ccccc8c7c6c6ccccc6c45)cc3)cc2)cc1] | 73% |
| 380 | 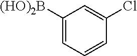 | 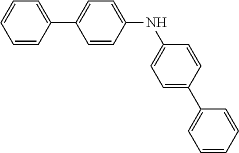 [SMILES: c1ccc(-c2ccc(Nc3ccc(-c4ccccc4)cc3)cc2)cc1] | 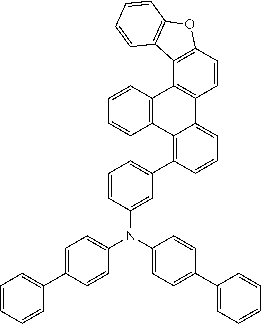 [SMILES: c1ccc(-c2ccc(N(c3ccc(-c4ccccc4)cc3)c3cccc(-c4cccc5c6ccc7oc8ccccc8c7c6c6ccccc6c45)c3)cc2)cc1] | 71% |
| 391 | 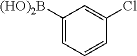 | 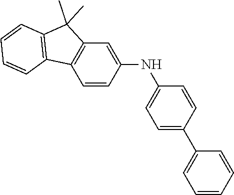 [SMILES: CC1(C)c2ccccc2-c2ccc(Nc3ccc(-c4ccccc4)cc3)cc21] | 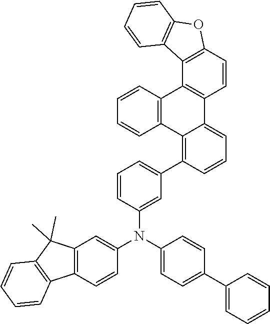 [SMILES: CC1(C)c2ccccc2-c2ccc(N(c3ccc(-c4ccccc4)cc3)c3cccc(-c4cccc5c6ccc7oc8ccccc8c7c6c6ccccc6c45)c3)cc21] | 69% |
| 398 | 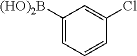 | 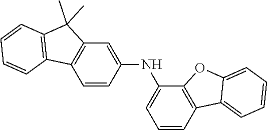 [SMILES: CC1(C)c2ccccc2-c2ccc(Nc3cccc4c3oc3ccccc34)cc21] | 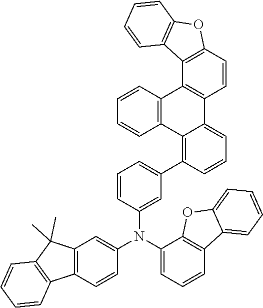 [SMILES: CC1(C)c2ccccc2-c2ccc(N(c3cccc(-c4cccc5c6ccc7oc8ccccc8c7c6c6ccccc6c45)c3)c3cccc4c3oc3ccccc34)cc21] | 70% |
| 415 | 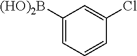 | 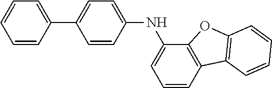 [SMILES: c1ccc(-c2ccc(Nc3cccc4c3oc3ccccc34)cc2)cc1] | 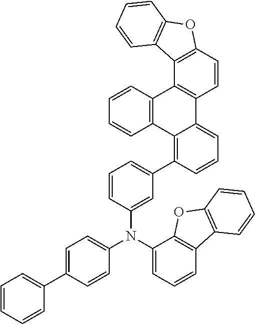 [SMILES: c1ccc(-c2ccc(N(c3cccc(-c4cccc5c6ccc7oc8ccccc8c7c6c6ccccc6c45)c3)c3cccc4c3oc3ccccc34)cc2)cc1] | 71% |
| 418 | 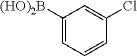 | 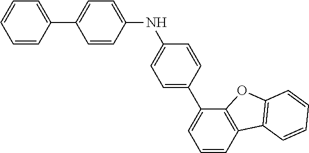 [SMILES: c1ccc(-c2ccc(Nc3ccc(-c4cccc5c4oc4ccccc45)cc3)cc2)cc1] | 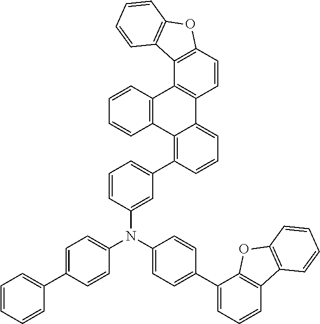 [SMILES: c1ccc(-c2ccc(N(c3ccc(-c4cccc5c4oc4ccccc45)cc3)c3cccc(-c4cccc5c6ccc7oc8ccccc8c7c6c6ccccc6c45)c3)cc2)cc1] | 73% |
| 440 | 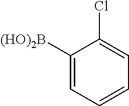 | 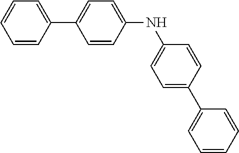 [SMILES: c1ccc(-c2ccc(Nc3ccc(-c4ccccc4)cc3)cc2)cc1] | 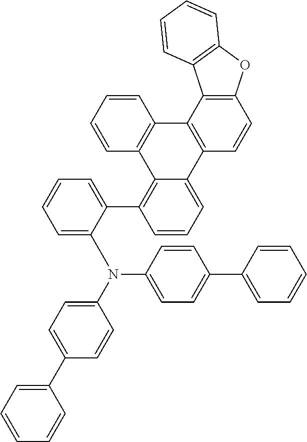 [SMILES: c1ccc(-c2ccc(N(c3ccc(-c4ccccc4)cc3)c3ccccc3-c3cccc4c5ccc6oc7ccccc7c6c5c5ccccc5c34)cc2)cc1] | 75% |
| 451 | 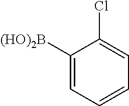 | 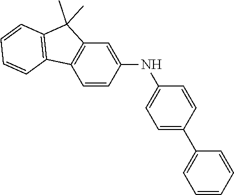 [SMILES: CC1(C)c2ccccc2-c2ccc(Nc3ccc(-c4ccccc4)cc3)cc21] | 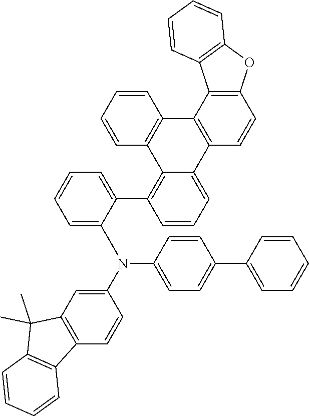 [SMILES: CC1(C)c2ccccc2-c2ccc(N(c3ccc(-c4ccccc4)cc3)c3ccccc3-c3cccc4c5ccc6oc7ccccc7c6c5c5ccccc5c34)cc21] | 74% |
| 458 | 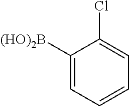 | 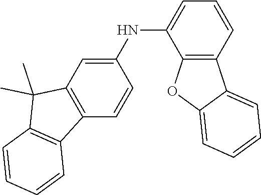 [SMILES: CC1(C)c2ccccc2-c2ccc(Nc3cccc4c3oc3ccccc34)cc21] | 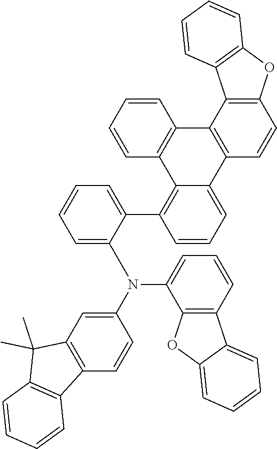 [SMILES: CC1(C)c2ccccc2-c2ccc(N(c3ccccc3-c3cccc4c5ccc6oc7ccccc7c6c5c5ccccc5c34)c3cccc4c3oc3ccccc34)cc21] | 72% |
| 475 | 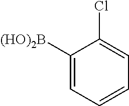 | 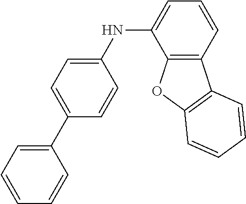 [SMILES: c1ccc(-c2ccc(Nc3cccc4c3oc3ccccc34)cc2)cc1] | 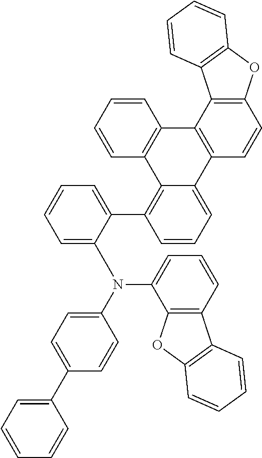 [SMILES: c1ccc(-c2ccc(N(c3ccccc3-c3cccc4c5ccc6oc7ccccc7c6c5c5ccccc5c34)c3cccc4c3oc3ccccc34)cc2)cc1] | 70% |

The remaining compounds other than the compounds described in Preparation Examples 1 to 3 and Tables 1 to 3 were prepared in the same manner as in the above-mentioned Preparation Examples, and the results of the synthesis of the compounds are shown in Tables 4 and 5 below. Table 4 below is the measurement value of ¹H NMR (CDCl₃, 400 Mz), and Table 5 below is the measurement value of the FD-mass spectrometer (FD-MS: Field desorption mass spectrometry).

TABLE 4

| Com- | |
|---|---|
| pound | ¹H NMR (CDCl₃, 300 MHz) |
| 12 | δ = 8.98 (1H, d), 8.71 (1H, s), 8.27 (1H, d), 8.11-8.07 (2H, |
| | m), 7.98 (1H, d), 7.71-7.31 (22H, m), 7.11 (1H, s) |
| 20 | δ = 8. 98 (1H, d), 8.71 (1H, s), 8.27 (1H, d), 8.11-8.07 (2H, |
| | m), 7.98 (1H, d), 7.82-7.31 (25H, m) |
| 25 | δ = 8.98 (1H, d), 8.71 (1H, s), 8.27 (1H, d), 8.11-8.07 (2H, |
| | m), 7.98 (1H, d), (29H, m) |
| 29 | δ = 8.98 (1H, d), 8.71 (1H, s), 8.27 (1H, d), 8.11-8.07 (2H, |
| | m), 7.98 (1H, d), 7.90-7.54 (10H, m), 7.45-7.28 (8H, m), |
| | 7.16 (1H, d), 7.11 (1H, s), 1.69 (6H, s) |
| 31 | δ = 8.98 (1H, d), 8.71 (1H, s), 8.27 (1H, d), 8.11-8.07 (2H, |
| | m), 7.98 (1H, d), 7.90-7.28 (23H, m), 7.16 (1H, d), 1.69 |
| | (6H, s) |
| 35 | δ = 8. 98 (1H, d), 8.71 (1H, s), 8.27 (1H, d), 8.11-8.07 (2H, |
| | m), 7.98 (1H, d), 7.90-7.82 (4H, m), 7.68-7.54 (7H, m), |
| | 7.39-7.28 (8H, m), 7.16 (1H, d), 7.06 (1H, d), 1.69 (6H, s) |
| 38 | δ = 8.98 (1H, d), 8.71 (1H, s), 8.27 (1H, d), 8.11-8.07 (2H, |
| | m), 7.98 (2H, d), 7.90-7.82 (3H, m), 7.68-7.54 (7H, m), |
| | 7.39-7.28 (8H, m), 7.16 (1H, d), 6.97 (1H, d), 1.69 (6H, s) |
| 43 | δ = 8.98-8.95 (2H, m), 8.71 (1H, s), 8.50 (1H, d), 8.27-8.20 |
| | (2H, m), 8.11-8.07 (3H, m), 7.98 (1H, d), 7.90-7.52 (12H, |
| | m), 7.39-7.28 (8H, m), 7.16 (1H, d), 1.69 (6H, s) |
| 47 | δ = 8.98 (1H, d), 8.71 (1H, s), 8.27 (1H, d), 8.11-8.07 (2H, |
| | m), 7.98 (1H, d), 7.90-7.18 (33H, m) |
| 55 | δ = 8. 98 (1H, d), 8.71 (1H, s), 8.27 (1H, d), 8.11-8.07 (2H, |
| | m), 7.98 (1H, d), 7.82-7.28 (21H, m), 6.97 (1H, d) |
| 56 | δ = 8.98 (1H, d), 8.71 (1H, s), 8.27 (1H, d), 8.11-8.07 (2H, |
| | m), 7.98 (2H, d), 7.82-7.17 (20H, m), 6.97 (1H, d) |
| 72 | δ = 9.27 (1H, s), 8.98 (1H, d), 8.37-8.27 (3H, m), 8.11 (1H, |
| | d), 7.98 (1H, d), 7.82-7.31 (25H, m), 7.11 (1H, s) |
| 80 | δ = 9.27 (1H, s), 8.98 (1H, d), 8.37-8.27 (3H, m), 8.11 (1H, |
| | d), 7.98 (1H, d), 7.82-7.31 (27H, m) |
| 91 | δ = 9.27 (1H, s), 8.98 (1H, d), 8.37-8.27 (3H, m), 8.11 (1H, |
| | d), 7.98 (1H, d), 7.82-7.28 (25H, m), 7.16 (1H, d), 1.69 |
| | (6H, s) |
| 98 | δ = 9.27 (1H, s), 8.98 (1H, d), 8.37-8.27 (3H, m), 8.11 (1H, |
| | d), 7.98 (2H, d), 7.90-7.82 (3H, m), 7.68-7.54 (8H, m), |
| | 7.39-7.28 (10H, m), 7.16 (1H, d), 6.97 (1H, d) |
| 115 | δ = 9.27 (1H, s), 8.98 (1H, d), 8.37-8.27 (3H, m), 8.11 (1H, |
| | d), 7.98 (2H, d), 7.82-7.28 (24H, m), 6.97 (1H, d) |
| 122 | δ = 9.27 (1H, s), 8.98 (1H, d), 8.37-8.27 (3H, m), 8.11 (1H, |
| | d), 7.98 (1H, d), 7.82-7.11 (22H, m) |
| 140 | δ = 9.27 (1H, s), 8.98 (1H, d), 8.37-8.27 (3H, m), 8.11 (1H, |
| | d), 7.98 (1H, d), 7.82-7.31 (26H, m), 7.18-7.17 (2H, m) |
| 148 | δ = 9.27 (1H, s), 8.98 (1H, d), 8.37-8.27 (3H, m), 8.11 (1H, |
| | d), 7.98 (1H, d), 7.90-7.82 (3H, m), 7.68-7.54 (5H, m), |
| | 7.39-7.00 (14H, m), 1.69 (6H, s) |
| 151 | δ = 9.27 (1H, s), 8.98 (1H, d), 8.37-8.27 (3H, m), 8.11 (1H, |
| | d), 7.98 (1H, d), 7.90-7.31 (24H, m), 7.18-7.16 (3H, m) |
| 158 | δ = 9.27 (1H, s), 8.98 (1H, d), 8.37-8.27 (3H, m), 8.11 (1H, |
| | d), 7.98 (2H, d), 7.90-7.82 (3H, m), 7.68-7.54 (7H, m), |
| | 7.39-7.27 (9H, m), 7.18-7.16 (3H, m), 6.97 (1H, d), 1.69 |
| | (6H, m) |
| 175 | δ = 9.27 (1H, s), 8.98 (1H, d), 8.37-8.27 (3H, m), 8.11 (1H, |
| | d), 7.98 (2H, d), 7.82-7.28 (22H, m), 7.18-7.17 (2H, m), |
| | 6.97 (1H, d) |
| 178 | δ = 9.27 (1H, s), 8.98 (1H, d), 8.37-8.27 (3H, m), 8.11-7.98 |
| | (5H, m), 7.82-7.27 (25H, m), 7.18-7.17 (2H, m) |
| 200 | δ = 9.27 (1H, s), 8.98 (1H, d), 8.37-8.27 (3H, m), 8.11-8.10 |
| | (2H, m), 7.98 (1H, d), 7.82-7.37 (26H, m), 7.14 (1H, m) |
| 211 | δ = 9.27 (1H, s), 8.98 (1H, d), 8.37-8.27 (3H, m), 8.11-8.10 |
| | (2H, m), 7.98 (1H, d), 7.90 (23H, m), 7.16-7.14 (2H, m) |
| 218 | δ = 9.27 (1H, s), 8.98 (1H, d), 8.37-8.27 (3H, m), 8.11-8.10 |
| | (2H, m), 7.98 (2H, d), 7.90-7.82 (3H, m), 7.68-7.54 (6H, |
| | m), 7.39-7.28 (10H, m) 7.16-7.14 (2H, m), 6.97 (1H, d), 1.69 |
| | (6H, s) |
| 235 | δ = 9.27 (1H, s), 8.98 (1H, d), 8.37-8.27 (3H, m), 8.11-8.10 |
| | (2H, m), 7.98 (2H, d), 7.82-7.28 (22H, m), 7.14 (1H, m), |
| | 6.97 (1H, d) |
| 242 | δ = 8.98 (1H, d), 8.17-8.11 (2H, m), 7.98 (1H, d), 7.83-7.24 |
| | (17H, m), 7.11-7.00 (4H, m) |
| 260 | δ = 8.98 (1H, d), 8.17-8.11 (2H, m), 7.98 (1H, d), 7.82-7.31 |
| | (27H, m) |
| 265 | δ = 8.98 (1H, d), 8.17-8.11 (2H, m), 7.98 (1H, d), 7.83-7.31 |
| | (31H, m) |
| 268 | δ = 8.98 (1H, d), 8.17-8.11 (2H, m), 7.98 (1H, d), 7.90-7.82 |
| | (4H, m), 7.68-7.54 (6H, m), 7.39-7.16 (8H, m), 7.08-7.00 |
| | (3H, m), 1.69 (6H, s) |
| 269 | δ = 8.98 (1H, d), 8.17-8.11 (2H, m), 7.98 (1H, d), 7.90-7.28 |
| | (21H, m), 7.16-7.11 (2H, m), 1.69 (6H, s) |
| 271 | δ = 8.98 (1H, d), 8.17-8.11 (2H, m), 7.98 (1H, d), 7.90-7.28 |
| | (24H, m), 7.16 (1H, d), 1.69 (6H, s) |
| 278 | δ = 8.98 (1H, d), 8.17-8.11 (2H, m), 7.98 (2H, d), 7.90-7.82 |
| | (4H, m), 7.68-7.54 (8H, m), 7.39-7.28 (8H, m), 7.16 (1H, |
| | d), 6.97 (1H, d), 1.69 (6H, s) |
| 295 | δ = 8.98 (1H, d), 8.17-8.11 (2H, m), 7.98 (2H, d), 7.83-7.28 |
| | (23H, m), 6.97 (1H, d) |
| 298 | δ = 8.98 (1H, d), 8.17-7.98 (6H, m), 7.83-7.31 (26H, m) |
| 302 | δ = 9.08 (1H, d), 8.98 (1H, d), 8.29-8.11 (4H, m), 7.98 (1H, |
| | d), 7.82-7.24 (18H, m), 7.11-7.00 (4H, m) |
| 320 | δ = 9.08 (1H, d), 8.98 (1H, d), 8.29-8.11 (4H, m), 7.98 (1H, |
| | d), 7.82-7.31 (28H, m) |
| 331 | δ = 9.08 (1H, d), 8.98 (1H, d), 8.29-8.11 (4H, m), 7.98 (1H, |
| | d), 7.82-7.28 (25H, m), 7.16 (1H, d), 1.69 (6H, s) |
| 338 | δ = 9.08 (1H, d), 8.98 (1H, d), 8.29-8.11 (4H, m), 7.98 (2H, |
| | d), 7.90-7.82 (3H, m), 7.68-7.54 (8H, m), 7.39-7.28 (10H, |
| | m), 7.16 (1H, d), 6.97 (1H, d), 1.69 (6H, s) |
| 355 | δ = 9.08 (1H, d), 8.98 (1H, d), 8.29-8.11 (4H, m), 7.98 (2H, |
| | d), 7.82-7.28 (24H, m), 6.97 (1H, d) |
| 358 | δ = 9.08 (1H, d), 8.98 (1H, d), 8.29-7.98 (8H, m), 7.82-7.31 |
| | (27H, m) |
| 380 | δ = 9.08 (1H, d), 8.98 (1H, d), 8.29-8.11 (4H, m), 7.98 (1H, |
| | d), 7.82-7.27 (26H, m), 7.18-7.17 (2H, m) |
| 391 | δ = 9.08 (1H, d), 8.98 (1H, d), 8.29-8.11 (4H, m), 7.98 (1H, |
| | d), 7.90-7.28 (23H, m), 7.18-7.17 (2H, m), 1.69 (6H, s) |
| 398 | δ = 9.08 (1H, d), 8.98 (1H, d), 8.29-8.11 (4H, m), 7.98 (2H, |
| | d), 7.90-7.82 (3H, m), 7.68-7.54 (7H, m), 7.39-7.27 (9H, |
| | m), 7.18-7.16 (3H, m), 6.97 (1H, d), 1.69 (6H, s) |
| 415 | δ = 9.08 (1H, d), 8.98 (1H, d), 8.29-8.11 (4H, m), 7.98 (2H, |
| | d), 7.82-7.28 (22H, m), 7.18-7.17 (2H, m), 6.97 (1H, d) |
| 418 | δ = 9.08 (1H, d), 8.98 (1H, d), 8.29-7.98 (8H, m), 7.82-7.27 |
| | (25H, m), 7.18-7.17 (2H, m) |
| 440 | δ = 9.08 (1H, d), 8.98 (1H, d), 8.29-8.11 (4H, m), 7.98 (1H, |
| | d), 7.82-7.31 (26H, m), 7.14 (1H, m) |
| 451 | δ = 9.08 (1H, d), 8.98 (1H, d), 8.29-8.11 (5H, m), 7.98 (1H, |
| | d), 7.90-7.28 (23H, m), 7.14 (1H, m), 1.69 (6H, s) |
| 458 | δ = 9.08 (1H, d), 8.98 (1H, d), 8.29-8.11 (5H, m), 7.98 (2H, |
| | d), 7.90-7.82 (3H, m), 7.68-7.54 (6H, m), 7.39-7.28 (10H, |
| | m), 7.16 (1H, d), 6.97 (1H, d), 1.69 (6H, s) |
| 475 | δ = 9.08 (1H, d), 8.98 (1H, d), 8.29-8.11 (5H, m), 7.98 (2H, |
| | d), 7.82-7.31 (22H, m), 7.14 (1H, m), 6.97 (1H, d) |

TABLE 5

| Compound | FD-MS | Compound | FD-MS |
|---|---|---|---|
| 12 | m/z = 611.22 | 20 | m/z = 637.24 |
| 25 | m/z = 713.27 | 29 | m/z = 651.26 |
| 31 | m/z = 677.27 | 35 | m/z = 717.30 |
| 38 | m/z = 691.25 | 43 | m/z = 727.29 |
| 47 | m/z = 801.30 | 55 | m/z = 651.22 |
| 56 | m/z = 651.22 | 72 | m/z = 687.26 |
| 80 | m/z = 713.27 | 91 | m/z = 753.30 |
| 98 | m/z = 762.28 | 115 | m/z = 727.25 |
| 118 | m/z = 803.28 | 122 | m/z = 611.22 |
| 140 | m/z = 713.27 | 148 | m/z = 677.27 |
| 151 | m/z = 753.30 | 158 | m/z = 767.28 |
| 175 | m/z = 727.25 | 178 | m/z = 803.28 |
| 200 | m/z = 713.27 | 211 | m/z = 753.30 |
| 218 | m/z = 767.28 | 235 | m/z = 727.25 |
| 242 | m/z = 535.19 | 260 | m/z = 637.24 |
| 265 | m/z = 713.27 | 268 | m/z = 601.24 |
| 269 | m/z = 651.26 | 271 | m/z = 677.27 |
| 278 | m/z = 691.25 | 295 | m/z = 651.22 |
| 298 | m/z = 727.25 | 302 | m/z = 611.22 |
| 320 | m/z = 713.27 | 331 | m/z = 753.30 |
| 338 | m/z = 767.28 | 355 | m/z = 875.32 |
| 358 | m/z = 803.28 | 380 | m/z = 713.27 |
| 391 | m/z = 753.30 | 398 | m/z = 767.28 |
| 415 | m/z = 727.25 | 418 | m/z = 803.28 |
| 440 | m/z = 713.27 | 451 | m/z = 753.30 |
| 458 | m/z = 767.28 | 475 | m/z = 727.25 |

### Experimental Example 1

(1) Manufacturing of Organic Light Emitting Device

A glass substrate coated with a thin film of indium tin oxide (ITO) with a thickness of 1,500 Å was ultrasonically washed with distilled water. After washing with distilled water, the substrate was ultrasonically washed with a solvent such as acetone, methanol, isopropyl alcohol, etc., dried, and then treated with ultraviolet ozone (WO) for 5 minutes using ultraviolet (UV) in a UV cleaner. Thereafter, the substrate was transferred to a plasma cleaner (PT), and then plasma-treated in a vacuum to increase the work function of ITO and remove the remaining film, and transferred to a thermal deposition equipment for organic deposition.

Subsequently, after evacuating the chamber until the vacuum level reaches 10⁻⁶ torr, a hole injection layer having a thickness of 600 Å was deposited on the ITO substrate by applying a current to the cell to evaporate 2-TNATA. The compound represented by Formula 1 shown in Table 6 below was put into another cell in the vacuum deposition equipment, and a current was applied to the cell to evaporate it and thus deposit a hole transport layer with a thickness of 300 Å on the hole injection layer.

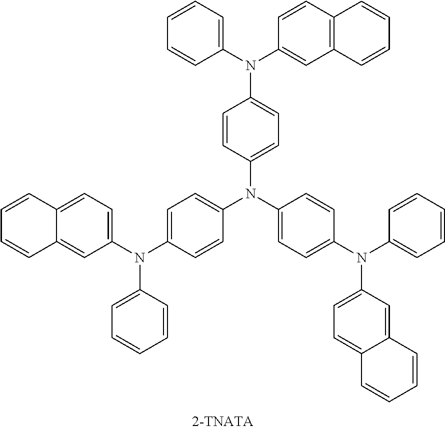 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

On top of that, a light emitting layer was thermally vacuum deposited as follows. The light emitting layer was formed by depositing the compound of 9-[4-(4,6-diphenyl-1,3,5-triazin-2-yl)phenyl]-9′-phenyl-3,3′-Bi-9H-carbazole as a host with a thickness of 400 Å and doping and depositing a green phosphorescent dopant [Ir(ppy) 3] with a thickness of 7% of the deposition thickness of the light emitting layer. Thereafter, bathocuproine (BCP) was deposited with a thickness of 60 Å as a hole blocking layer, and Alq₃ was deposited with a thickness of 200 Å as an electron transport layer on it. Finally, on the electron transport layer, lithium fluoride (LiF) was deposited with a thickness of 10 Å to form an electron injection layer, and then aluminum (Al) was deposited with a thickness of 1,200 Å on the electron injection layer to form a negative electrode, thereby manufacturing an organic light emitting device.

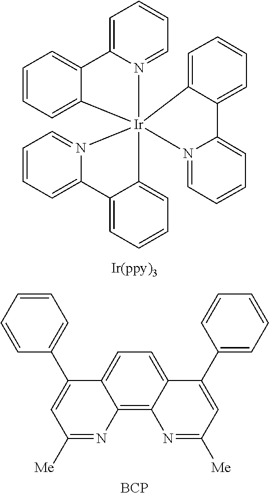

On the other hand, all organic compounds required for the manufacture of OLED devices were purified by vacuum sublimation under 10⁻⁶ to 10⁻⁸ torr for each material, and used for the manufacture of OLED.

In this case, the comparative compounds used in the hole transport layer of Comparative Examples below are as follows:

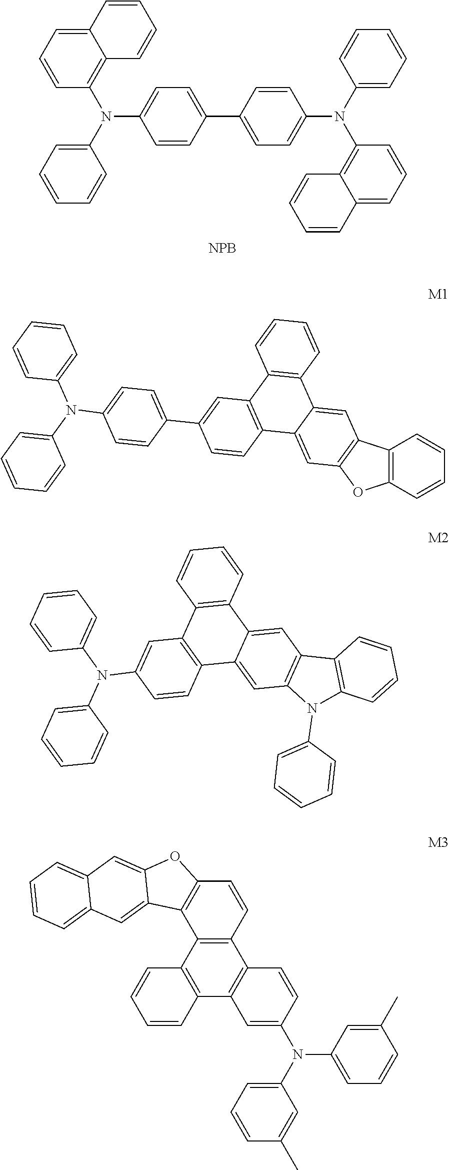 [SMILES: c1ccc(N(c2ccc(-c3ccc(N(c4ccccc4)c4cccc5ccccc45)cc3)cc2)c2cccc3ccccc23)cc1 ; "NPB" ; c1ccc(N(c2ccccc2)c2ccc(-c3ccc4c(c3)c3ccccc3c3cc5c(cc43)oc3ccccc35)cc2)cc1 ; c1ccc(N(c2ccccc2)c2ccc3c(c2)c2ccccc2c2cc4c5ccccc5n(-c5ccccc5)c4cc32)cc1 ; Cc1cccc(N(c2cccc(C)c2)c2ccc3c(c2)c2ccccc2c2c3ccc3oc4cc5ccccc5cc4c32)c1]

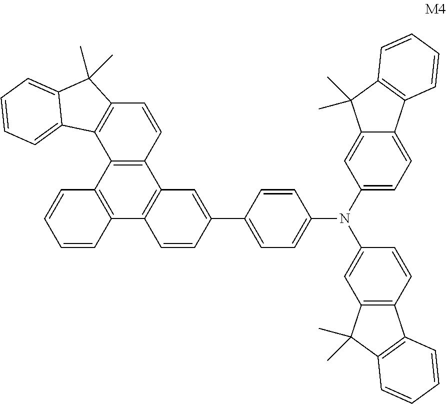 [SMILES: CC1(C)c2ccccc2-c2ccc(N(c3ccc(-c4ccc5c6ccccc6c6c7c(ccc6c5c4)C(C)(C)c4ccccc4-7)cc3)c3ccc4c(c3)C(C)(C)c3ccccc3-4)cc21]

(2) Operating Voltage and Luminous Efficiency of Organic Light Emitting Device

For the organic electroluminescence devices manufactured as described above, electroluminescence (EL) characteristics were measured with M7000 from McScience Co. With the measurement results, T₉₀ was measured at a reference luminance of 6,000 cd/m² through the lifetime measuring device (M6000) manufactured by McScience Co. T₉₀ means a lifetime (unit: hour (h)) that is a time at which the luminance becomes 90% compared to the initial luminance.

The characteristics of the organic electroluminescence device of the present invention are shown in Table 6 below.

TABLE 6

| | | Operating | Luminous | |
|---|---|---|---|---|
| | | voltage | efficiency | Lifetime |
| | Compound | (V) | (cd/A) | (T₉₀) |
| Example 1 | 12 | 4.09 | 125.63 | 139 |
| Example 2 | 20 | 3.87 | 123.39 | 138 |
| Example 3 | 25 | 4.04 | 122.83 | 138 |
| Example 4 | 29 | 4.00 | 122.27 | 138 |
| Example 5 | 31 | 3.96 | 121.70 | 139 |
| Example 6 | 35 | 3.91 | 121.14 | 140 |
| Example 7 | 47 | 3.87 | 120.58 | 140 |
| Example 8 | 55 | 3.93 | 120.02 | 141 |
| Example 9 | 72 | 3.96 | 119.46 | 142 |
| Example 10 | 80 | 3.98 | 118.90 | 143 |
| Example 11 | 91 | 4.00 | 118.34 | 143 |
| Example 12 | 98 | 4.02 | 117.78 | 144 |
| Example 13 | 115 | 4.04 | 118.34 | 145 |
| Example 14 | 122 | 4.06 | 118.90 | 145 |
| Example 15 | 140 | 4.09 | 119.46 | 144 |
| Example 16 | 148 | 3.87 | 120.02 | 143 |
| Example 17 | 151 | 3.89 | 120.58 | 143 |
| Example 18 | 200 | 3.91 | 121.14 | 142 |
| Example 19 | 211 | 3.93 | 121.70 | 141 |
| Example 20 | 242 | 3.96 | 122.27 | 141 |
| Example 21 | 260 | 3.98 | 122.83 | 140 |
| Example 22 | 265 | 4.00 | 123.39 | 140 |
| Example 23 | 268 | 4.02 | 123.95 | 139 |
| Example 24 | 269 | 4.04 | 124.51 | 147 |
| Example 25 | 271 | 4.06 | 123.39 | 144 |
| Example 26 | 295 | 4.09 | 122.83 | 143 |
| Example 27 | 302 | 4.06 | 122.27 | 143 |
| Example 28 | 320 | 4.04 | 121.70 | 142 |
| Example 29 | 331 | 4.02 | 121.14 | 141 |
| Example 30 | 380 | 4.00 | 120.58 | 141 |
| Example 31 | 391 | 3.98 | 120.02 | 140 |
| Example 32 | 440 | 3.96 | 119.46 | 140 |
| Example 33 | 451 | 3.93 | 118.90 | 139 |
| Comparative | NPB | 4.55 | 101.27 | 117 |
| Example 1 | | | | |
| Comparative | M1 | 4.35 | 112.17 | 131 |
| Example 2 | | | | |
| Comparative | M2 | 4.31 | 111.57 | 129 |
| Example 3 | | | | |
| Comparative | M3 | 4.30 | 110.32 | 126 |
| Example 4 | | | | |
| Comparative | M4 | 4.30 | 110.37 | 124 |
| Example 5 | | | | |

As can be seen from the results of the above Table 6, it was confirmed in the green organic light emitting device that the organic light emitting device using the material for the hole transport layer containing the heterocyclic compound according to the present invention had a lower operating voltage and significantly improved luminous efficiency and lifetime compared to Comparative Examples.

It was confirmed that if the heterocyclic compound (amine derivative) according to the present invention used in Example of Table 6 was used as the hole transport layer, the unshared electron pair of the amine improved the flow of the holes, thereby improving the hole transport ability of the hole transport layer. In addition, it was confirmed that in the case of the heterocyclic compound (amine derivative) according to the present invention, the thermal stability of the compound was improved by combining the amine moiety and the substituent with enhanced hole characteristics and thus increasing the planarity and glass transition temperature of the amine derivative.

In addition, it was confirmed that through the control of the band gap and triplet energy level (T1 level), the hole transport ability is improved, and the stability of the molecule is also increased, thereby lowering the operating voltage of the organic light emitting device and improving the light efficiency, and the lifetime characteristics of the organic light emitting device is improved by the improved thermal stability of the compound.

### Experimental Example 2

(1) Manufacturing of Organic Light Emitting Device

The transparent electrode ITO (Indium Tin Oxide) thin film obtained from glass for OLED (manufactured by Samsung-Corning) was ultrasonically washed for 5 minutes, respectively, using trichloroethylene, acetone, ethanol and distilled water sequentially, and then stored in isopropanol and used. Next, the ITO substrate is installed in the substrate folder of the vacuum deposition equipment, and the following 4,4′,4″-tris(N,N-(2-naphthyl)-phenylamino)triphenyl amine (2-TNATA) was placed in the cell in the vacuum deposition equipment.

Subsequently, after evacuating the chamber until the vacuum level reaches 10⁻⁶ torr, a hole injection layer having a thickness of 600 Å was deposited on the ITO substrate by applying a current to the cell to evaporate 2-TNATA. The following N,N′-bis(α-naphthyl)-N,N′-diphenyl-4,4′-diamine (NPB) was put into another cell in the vacuum deposition equipment, and a current was applied to the cell to evaporate it and thus deposit a hole transport layer with a thickness of 150 Å on the hole injection layer. Thereafter, the compound shown in Table 7 below was deposited on the hole transport layer with a thickness of 50 Å to form an electron blocking layer.

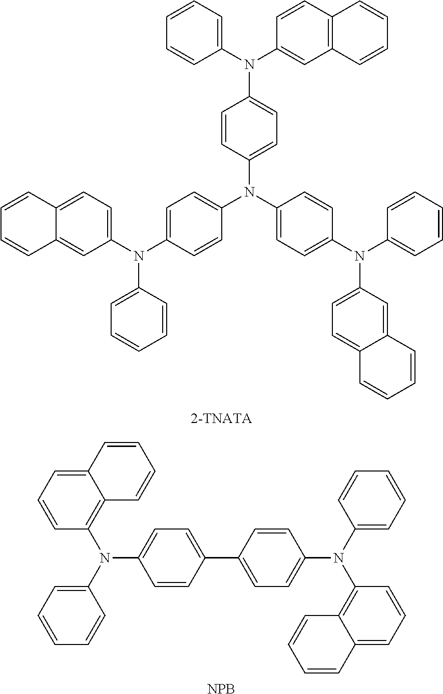 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

In this way, after forming the hole injection layer and the hole transport layer, a blue light emitting material having the following structure was deposited as a light emitting layer thereon. Specifically, H1, which is a blue light-emitting host material, was vacuum-deposited with a thickness of 200 Å in one cell in the vacuum deposition equipment, and D1, which is a blue light emitting dopant material, was vacuum-deposited thereon at 5% compared to the host material.

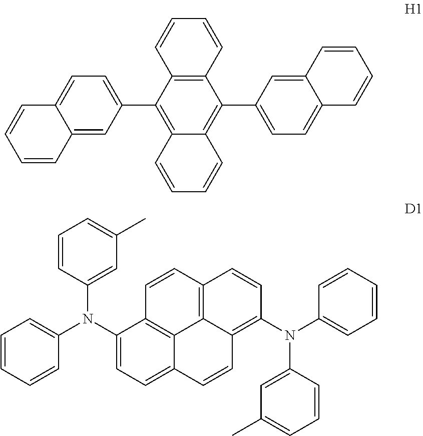

Subsequently, as an electron transport layer, a compound of the following Structural Formula E1 was deposited with a thickness of 300 Å.

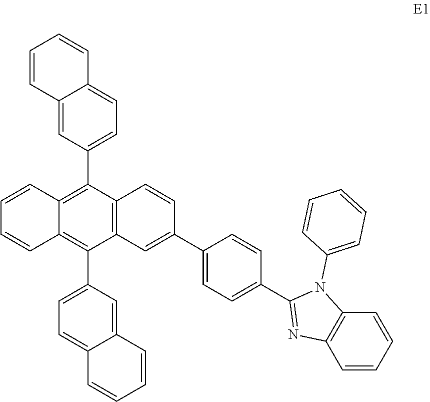 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Thereafter, lithium fluoride (LiF) was deposited with a thickness of 10 Å as an electron injection layer, and aluminum (Al) with a thickness of 1,000 Å was used to manufacture an OLED device.

On the other hand, all organic compounds necessary for the manufacture of OLED devices were purified by vacuum sublimation under 10⁻⁶ to 10⁻⁸ torr for each material and then used in the manufacture of the OLED devices.

In this case, the comparative compounds used in the electron blocking layer of Comparative Examples below are as follows:

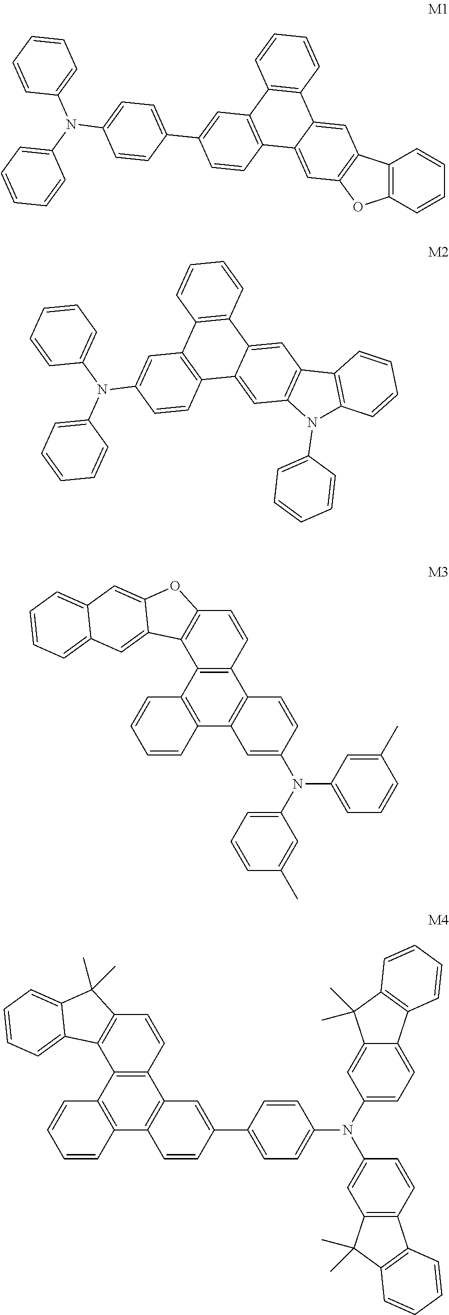 [SMILES: c1ccc(N(c2ccccc2)c2ccc(-c3ccc4c(c3)c3ccccc3c3cc5c(cc43)oc3ccccc35)cc2)cc1 ; c1ccc(N(c2ccccc2)c2ccc3c(c2)c2ccccc2c2cc4c5ccccc5n(-c5ccccc5)c4cc32)cc1 ; Cc1cccc(N(c2cccc(C)c2)c2ccc3c(c2)c2ccccc2c2c3ccc3oc4cc5ccccc5cc4c32)c1 ; CC1(C)c2ccccc2-c2ccc(N(c3ccc(-c4ccc5c6ccccc6c6c7c(ccc6c5c4)C(C)(C)c4ccccc4-7)cc3)c3ccc4c(c3)C(C)(C)c3ccccc3-4)cc21]

(2) Operating Voltage and Luminous Efficiency of Organic light Emitting Device

For the organic light emitting devices manufactured as described above, electroluminescence (EL) characteristics were measured with M7000 from McScience Co. With the measurement results, T₉₅ was measured at a reference luminance of 6,000 cd/m 2 through the lifetime measuring device (M6000) manufactured by McScience Co. T₉₅ means a lifetime (unit: Hour (h)) that is a time at which the luminance becomes 95% compared to the initial luminance.

The characteristics of the organic electroluminescence device of the present invention are shown in Table 7 below.

TABLE 7

| | | Operating | Luminous | |
|---|---|---|---|---|
| | | voltage | efficiency | Lifetime |
| | Compound | (V) | (cd/A) | (T₉₅) |
| Example 34 | 38 | 5.26 | 7.45 | 50 |
| Example 35 | 43 | 4.99 | 7.32 | 50 |
| Example 36 | 56 | 5.21 | 7.28 | 49 |
| Example 37 | 98 | 5.15 | 7.25 | 50 |
| Example 38 | 115 | 5.10 | 7.22 | 50 |
| Example 39 | 158 | 5.04 | 7.18 | 50 |
| Example 40 | 175 | 4.99 | 7.15 | 50 |
| Example 41 | 218 | 5.07 | 7.12 | 51 |
| Example 42 | 235 | 5.10 | 7.08 | 51 |
| Example 43 | 278 | 5.12 | 7.05 | 51 |
| Example 44 | 298 | 5.15 | 7.02 | 51 |
| Example 45 | 338 | 5.18 | 6.98 | 52 |
| Example 46 | 355 | 5.21 | 7.02 | 52 |
| Example 47 | 398 | 5.24 | 7.05 | 52 |
| Example 48 | 415 | 5.26 | 7.08 | 52 |
| Example 49 | 458 | 4.99 | 7.12 | 51 |
| Example 50 | 475 | 5.01 | 7.15 | 51 |
| Comparative | M1 | 5.62 | 6.41 | 45 |
| Example 6 | | | | |
| Comparative | M2 | 5.63 | 6.65 | 45 |
| Example 7 | | | | |
| Comparative | M3 | 5.55 | 6.56 | 44 |
| Example 8 | | | | |
| Comparative | M4 | 5.54 | 6.57 | 42 |
| Example 9 | | | | |

As can be seen from the results in Table 7 above, it was confirmed that in the case of the blue organic light emitting device, the organic light emitting device using the material for the electron blocking layer including the heterocyclic compound according to the present invention had a lower operating voltage and significantly improved luminous efficiency and lifetime compared to Comparative Examples.

Here, if electrons are not combined in the light emitting layer and moved to the positive electrode through the hole transport layer, there is a phenomenon that the efficiency and lifetime of the OLED device are reduced. In order to prevent such a phenomenon, if a compound having a high LUMO level is used as an electron blocking layer, the electrons trying to move through the light emitting layer to the positive electrode are blocked by the energy barrier of the electron blocking layer. Due to this, the probability that holes and electrons form excitons is increased, and the possibility that they are emitted as light from the light emitting layer is increased. Therefore, if the heterocyclic compound according to the present invention is used as an electron blocking layer, the organic light emitting device exhibits excellent performance in all aspects of operating voltage, efficiency and lifetime.

In particular, it was confirmed that with respect to the heterocyclic compound (amine derivative) according to the present invention, if the amine derivative is used as a hole transport layer, the unshared electron pair of the amine can improve the flow of holes and improve the hole transport ability of the hole transport layer, and if the amine derivative is used as an electron blocking layer, it is possible to suppress the degradation of the hole transport material caused by the electrons entering the hole transport layer, and also, the heterocyclic compound according to the present invention improves the thermal stability of the compound by combining an amine moiety and a substituent with enhanced hole characteristics and thus increasing the planarity and glass transition temperature of the amine derivative.

In addition, it was confirmed that through the control of the band gap and triplet energy level (T1 level), the hole transport ability is improved, and the stability of the molecule is also increased, thereby lowering the operating voltage of the organic light emitting device and improving the light efficiency, and the lifetime characteristics of the organic light emitting device is improved by the improved thermal stability of the compound.

### Experimental Example 3

(1) Manufacture of Organic Light Emitting Device

A glass substrate coated with a thin film of indium tin oxide (ITO) with a thickness of 1,500 Å was ultrasonically washed with distilled water. After washing with distilled water, the substrate was ultrasonically washed with a solvent such as acetone, methanol, isopropyl alcohol, etc., dried, and then treated with UVO for 5 minutes using ultraviolet (UV) in a UV cleaner. Thereafter, the substrate was transferred to a plasma cleaner (PT), and then plasma-treated in a vacuum to increase the work function of ITO and remove the remaining film, and transferred to a thermal deposition equipment for organic deposition.

A hole injection layer of 4,4′,4″-tris[2-naphthyl(phenyl)amino]triphenylamine (2-TNATA) and a hole transport layer of N,N′-Di(1-naphthyl)-N,N′-diphenyl-(1,1′-biphenyl)-4,4′-diamine (NPB) as common layers were formed on the ITO transparent electrode (positive electrode).

On top of that, a light emitting layer was thermally deposited in the vacuum as follows. The light emitting layer was deposited with a thickness of 500 aby using the compounds described in Table 7 below as a single host, or using n-host (n-type host) having good electron transport ability as the first host and the compound shown in Table 8 below as the second host in a manner of depositing two host compounds from one source, and doping the host with a red phosphorescent dopant [(Piq)₂(Ir)(acac)] in an amount of 3 wt. % of the host material or doping the host with a green phosphorescent dopant [Ir(ppy)₃] in an amount of 7 wt. % of the host material.

Thereafter, BCP was deposited with a thickness of 60 Å as a hole blocking layer, and Alq₃ was deposited with a thickness of 200 Å as an electron transport layer on it.

At this time, when two hosts are used, the compound used as the n-host is as follows.

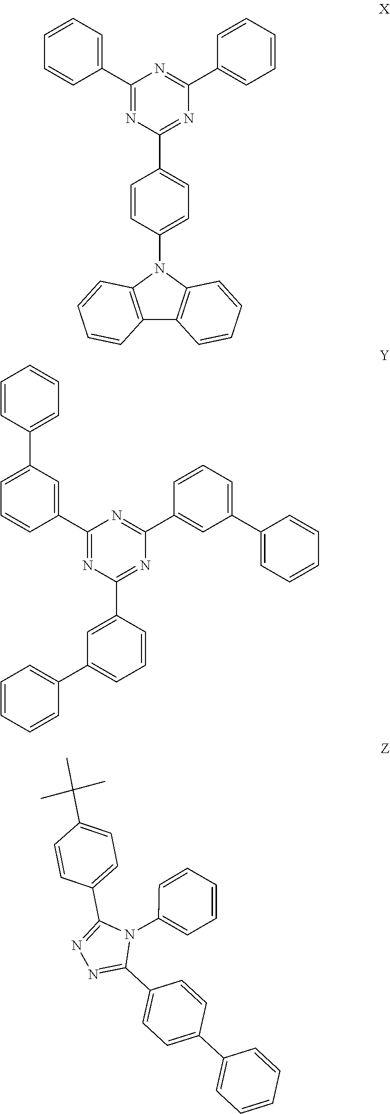

Finally, on the electron transport layer, lithium fluoride (LiF) was deposited with a thickness of 10 Å to form an electron injection layer, and then aluminum (Al) was deposited with a thickness of 1,200 Å on the electron injection layer to form a negative electrode, thereby manufacturing an organic light emitting device.

Specifically, the compounds used for the host in Examples 51 to 70 and Comparative Examples 10 to 15 are shown in Table 8 below.

In this case, compounds M1 to M3 used as hosts in Comparative Examples 10 to 15 in Table 8 below are as follows:

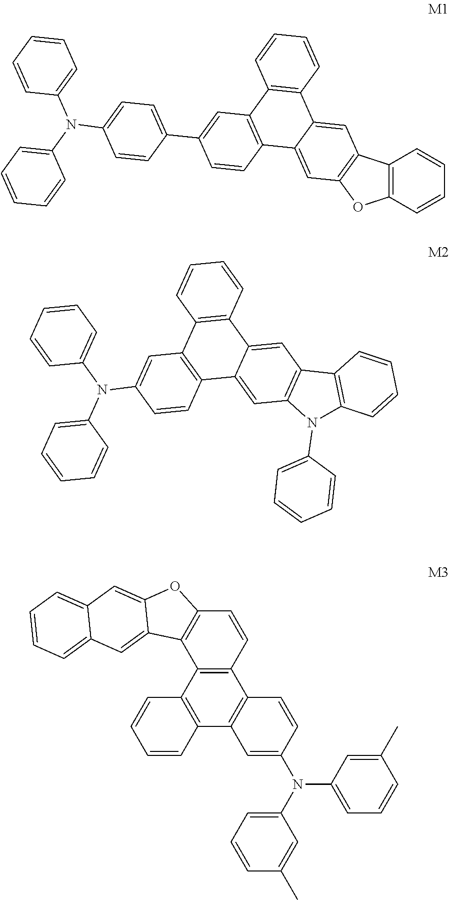 [SMILES: c1ccc(N(c2ccccc2)c2ccc(-c3ccc4c(c3)c3ccccc3c3cc5c(cc43)oc3ccccc35)cc2)cc1 ; c1ccc(N(c2ccccc2)c2ccc3c(c2)c2ccccc2c2cc4c5ccccc5n(-c5ccccc5)c4cc32)cc1 ; Cc1cccc(N(c2cccc(C)c2)c2ccc3c(c2)c2ccccc2c2c3ccc3oc4cc5ccccc5cc4c32)c1]

On the other hand, all organic compounds necessary for the manufacture of organic light emitting devices were purified by vacuum sublimation under 10⁻⁶ to 10⁻⁸ torr for each material and then used in the manufacture of the organic light emitting devices.

(2) Operating Voltage and Luminous Efficiency of Organic Light Emitting Device

For the organic light emitting devices manufactured as described above, electroluminescence (EL) characteristics were measured with M7000 from McScience Co. with the measurement results, T₉₅ was measured at a reference luminance of 6,000 cd/m 2 through the lifetime measuring device (M6000) manufactured by McScience Co. T₉₅ means a lifetime (unit: Hour (h)) that is a time at which the luminance becomes 95% compared to the initial luminance.

The results of measuring the operating voltage, luminous efficiency, luminous color, and lifetime of the organic light emitting device manufactured according to the present invention are shown in Table 8 below.

TABLE 8

| | | | Operating | Effi- | Lumi- | Life- |
|---|---|---|---|---|---|---|
| | First | Second | voltage | ciency | nous | time |
| | host | host | (V) | (cd/A) | color | (T₉₅) |
| Example 51 | 118 | | 4.02 | 42.1 | red | 113 |
| Example 52 | | | 3.95 | 67.6 | green | 85 |
| Example 53 | 178 | | 4.06 | 41.5 | red | 109 |
| Example 54 | | | 4.09 | 72.5 | green | 87 |
| Example 55 | 358 | | 3.99 | 41.8 | red | 118 |
| Example 56 | | | 3.92 | 68.1 | green | 99 |
| Example 57 | 418 | | 3.89 | 44.2 | red | 104 |
| Example 58 | | | 3.77 | 71.4 | green | 102 |
| Example 59 | X | 118 | 3.80 | 45.9 | red | 153 |
| Example 60 | | 178 | 3.83 | 47.4 | red | 174 |
| Example 61 | | 358 | 3.85 | 51.1 | red | 151 |
| Example 62 | | 418 | 3.97 | 49.3 | red | 168 |
| Example 63 | Y | 118 | 3.79 | 100.4 | green | 154 |
| Example 64 | | 178 | 3.84 | 97.5 | green | 151 |
| Example 65 | | 358 | 3.83 | 102.7 | green | 166 |
| Example 66 | | 418 | 3.95 | 111.2 | green | 152 |
| Example 67 | Z | 118 | 3.82 | 54.3 | red | 169 |
| Example 68 | | 178 | 3.87 | 45.2 | red | 170 |
| Example 69 | | 358 | 3.98 | 44.8 | red | 175 |
| Example 70 | | 418 | 3.78 | 49.9 | red | 149 |
| Comparative | M1 | | 4.43 | 21.5 | red | 52 |
| Example 10 | | | | | | |
| Comparative | X | M1 | 4.38 | 41.3 | red | 110 |
| Example 11 | | | | | | |
| Comparative | M2 | | 4.54 | 63.7 | green | 65 |
| Example 12 | | | | | | |
| Comparative | Y | M2 | 4.35 | 89.5 | green | 135 |
| Example 13 | | | | | | |
| Comparative | M3 | | 4.43 | 23.6 | red | 62 |
| Example 14 | | | | | | |
| Comparative | Z | M3 | 4.31 | 43.1 | red | 109 |
| Example 15 | | | | | | |

As can be seen from the results in Table 8 above, it was confirmed that in the case of the organic light emitting devices of Examples 51 to 58, in which the light emitting layers were formed using the heterocyclic compound according to the present invention as a single host material, the luminous efficiency and lifetime are superior to those of the organic light emitting devices of Comparative Examples 10, 12 and 14, which do not use the heterocyclic compound according to the present invention as a single host material.

In addition, it was confirmed that in the case of the organic light emitting devices of Examples 59 to 70, in which the light emitting layer was formed by using the first host material corresponding to n-host and the heterocyclic compound according to the present invention as the second host material corresponding to p-host at the same time, the luminous efficiency and lifetime are superior to the organic light emitting devices of Comparative Examples 11, 13 and 15, in which the light emitting layer was formed by using the first host material corresponding to the n-host and the compound other than the heterocyclic compound according to the present invention as the second host material corresponding to the p-host at the same time.

In addition, it was confirmed that in the case of the organic light emitting devices of Examples 51 to 58, in which the light emitting layer was formed by using the heterocyclic compound according to the present invention as a single host material, the luminous efficiency and lifetime are equal or superior to the organic light emitting devices of Comparative Examples 11, 13 and in which the light emitting layer was formed by using the first host material corresponding to the n-host and the compound other than the heterocyclic compound according to the present invention as a second host material corresponding to the p-host at the same time.

Generally, considering that the luminous efficiency and lifetime are excellent in the case of using an n-host (n-type host) having good electron transport capability as a first host and a g-host (p-type host) having good hole transport ability as a second host rather than using a single host material, it can be seen that if the heterocyclic compound according to the present invention is used as a host material, the luminous efficiency and lifetime of the organic light emitting device can be remarkably improved.

It is considered that the reason is that if the heterocyclic compound according to the present invention is used as a host material, holes and electrons from each charge transport layer can be efficiently injected into the light emitting layer. It is considered that the reason is also because, as described above, the size of the space and the orientation formed are affected by the interaction of materials during deposition.

On the other hand, it is considered that the efficient injection of holes and electrons into the light emitting layer is also a result of being influenced by the size of the space and the orientation formed by the interaction of materials during deposition, and is an effect caused by the difference in orientation characteristics and space size of the heterocyclic compound according to the present invention and the M1 to M3 as described above.

The present invention is not limited to the above Examples, but may be implemented in a variety of different forms, and it will be understood by those of ordinary skill in the art to which the present invention pertains, that the present invention may be embodied in other specific forms without changing the technical spirit or essential features of the present invention. Therefore, it should be understood that the Examples described above are intended to illustrate the present invention in all aspects and not to limit it.

All simple modifications and variations of the present invention fall within the scope of the present invention, and the specific protection scope of the present invention will be clarified by the appended claims.

### DESCRIPTION OF SYMBOL

100: Substrate
200: Positive electrode
300: Organic layer
301: Hole injection layer
302: Hole transport layer
303: Light emitting layer
304: Hole blocking layer
305: Electron transport layer
306: Electron injection layer
400: Negative electrode

1. A heterocyclic compound represented by Formula 1 below: 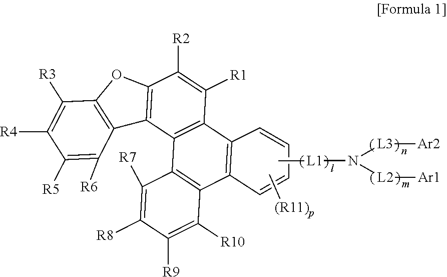
wherein,
Ar1 and Ar2 are the same as or different from each other, and are each independently a substituted or unsubstituted C6 to C60 aryl group; or a substituted or unsubstituted C2 to C60 heteroaryl group,
R1 to R11 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
L1 to L3 are the same as or different from each other, and are each independently a direct bond; a substituted or unsubstituted C6 to C60 arylene group; or a substituted or unsubstituted C2 to C60 heteroarylene group,
l, m and n are the same as or different from each other, and each independently are an integer of 0 to 5, and when l is 2 or more, each L1 is the same as or different from each other, and when m is 2 or more, each L2 is the same as or different from each other, and when n is 2 or more, each L3 is the same as or different from each other,
p is an integer of 1 to 3 and when p is 2 or more, each R11 is the same as or different from each other.

2. The heterocyclic compound according to claim 1, wherein Formula 1 is represented by Formula 1-1 or Formula 1-2 below: 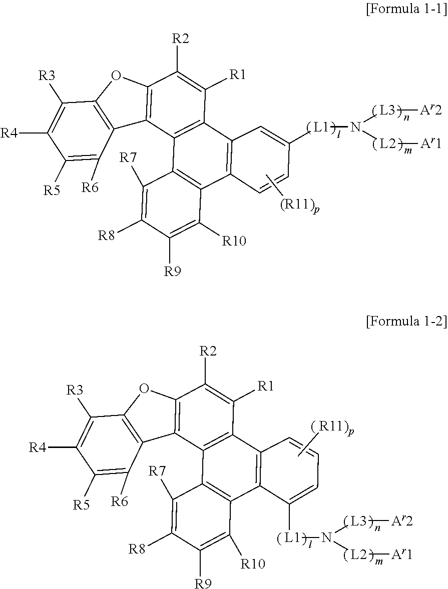
wherein Ar1, Ar2, R1 to R11, L1 to L3, l, m, n, and p are the same as defined in Formula 1 above.

3. The heterocyclic compound according to claim 1, wherein R1 to R11 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

4. The heterocyclic compound according to claim 1, wherein Formula 1 is represented by any one of the compounds below:

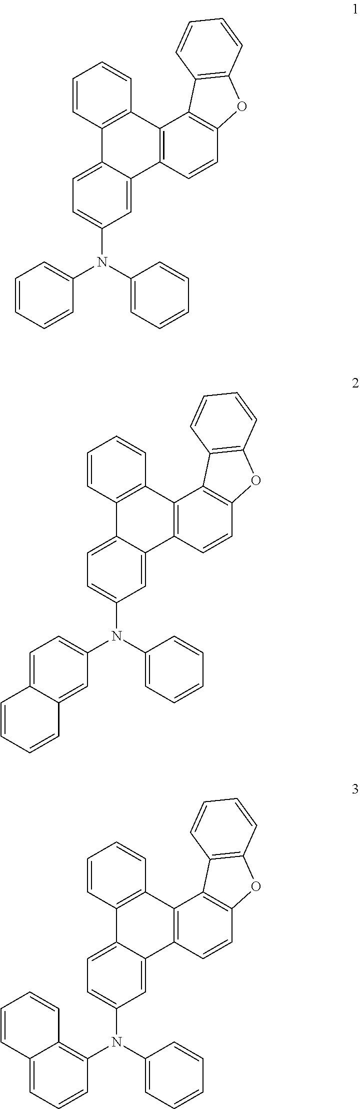 [SMILES: c1ccc(N(c2ccccc2)c2ccc3c4ccccc4c4c(ccc5oc6ccccc6c54)c3c2)cc1 ; c1ccc(N(c2ccc3ccccc3c2)c2ccc3c4ccccc4c4c(ccc5oc6ccccc6c54)c3c2)cc1 ; c1ccc(N(c2ccc3c4ccccc4c4c(ccc5oc6ccccc6c54)c3c2)c2cccc3ccccc23)cc1]

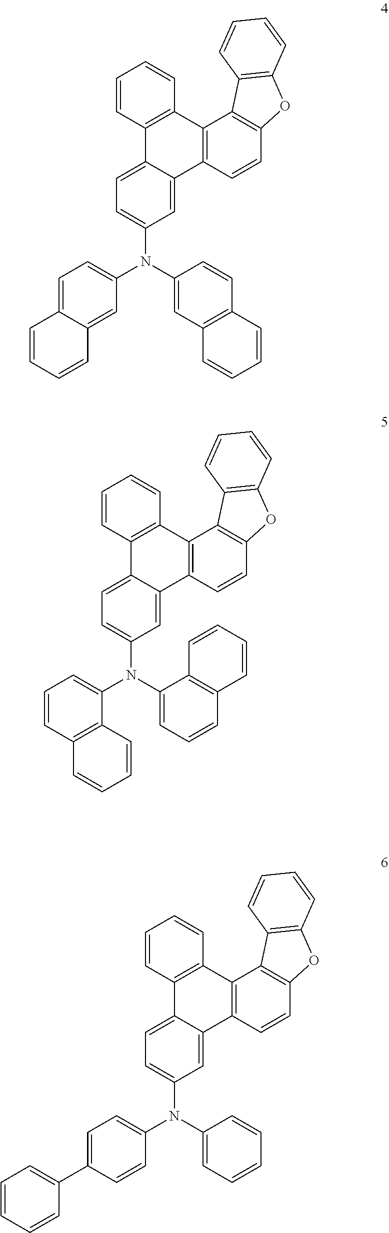 [SMILES: c1ccc2cc(N(c3ccc4ccccc4c3)c3ccc4c5ccccc5c5c(ccc6oc7ccccc7c65)c4c3)ccc2c1 ; c1ccc2c(N(c3ccc4c5ccccc5c5c(ccc6oc7ccccc7c65)c4c3)c3cccc4ccccc34)cccc2c1 ; c1ccc(-c2ccc(N(c3ccccc3)c3ccc4c5ccccc5c5c(ccc6oc7ccccc7c65)c4c3)cc2)cc1]

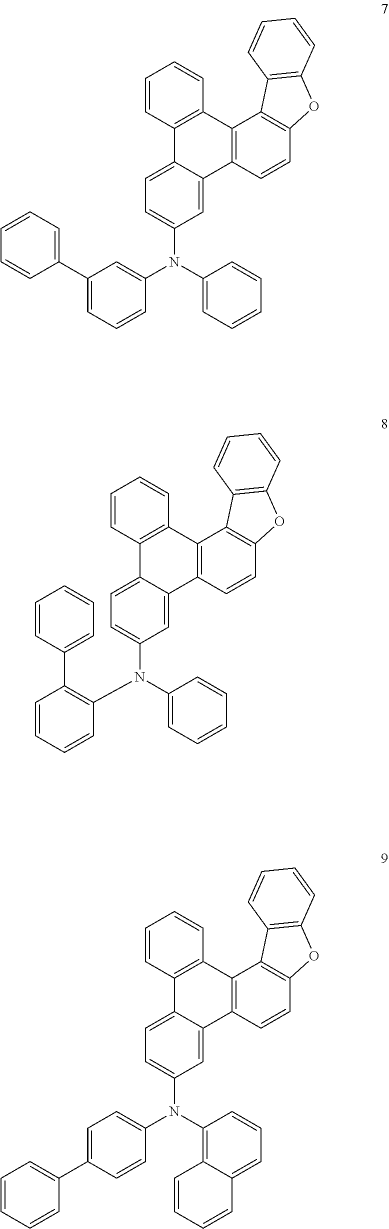 [SMILES: c1ccc(-c2cccc(N(c3ccccc3)c3ccc4c5ccccc5c5c(ccc6oc7ccccc7c65)c4c3)c2)cc1 ; c1ccc(-c2ccccc2N(c2ccccc2)c2ccc3c4ccccc4c4c(ccc5oc6ccccc6c54)c3c2)cc1 ; c1ccc(-c2ccc(N(c3ccc4c5ccccc5c5c(ccc6oc7ccccc7c65)c4c3)c3cccc4ccccc34)cc2)cc1]

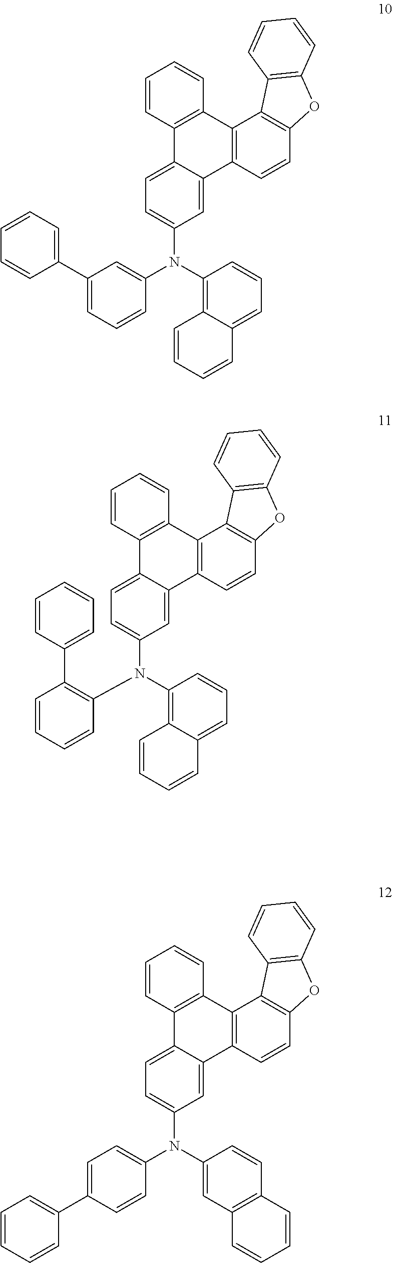 [SMILES: c1ccc(-c2cccc(N(c3ccc4c5ccccc5c5c(ccc6oc7ccccc7c65)c4c3)c3cccc4ccccc34)c2)cc1 ; c1ccc(-c2ccccc2N(c2ccc3c4ccccc4c4c(ccc5oc6ccccc6c54)c3c2)c2cccc3ccccc23)cc1 ; c1ccc(-c2ccc(N(c3ccc4ccccc4c3)c3ccc4c5ccccc5c5c(ccc6oc7ccccc7c65)c4c3)cc2)cc1]

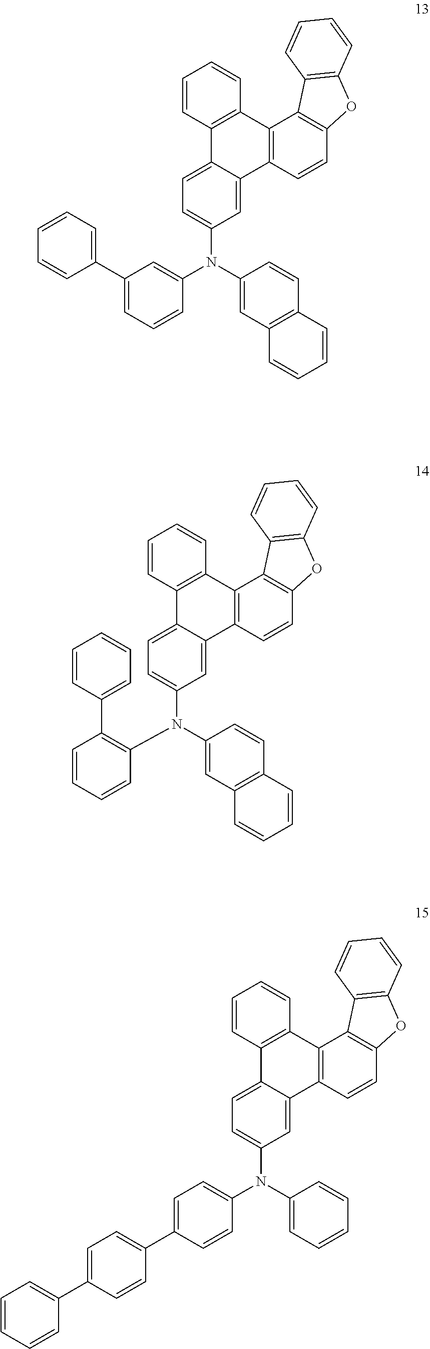 [SMILES: c1ccc(-c2cccc(N(c3ccc4ccccc4c3)c3ccc4c5ccccc5c5c(ccc6oc7ccccc7c65)c4c3)c2)cc1 ; c1ccc(-c2ccccc2N(c2ccc3ccccc3c2)c2ccc3c4ccccc4c4c(ccc5oc6ccccc6c54)c3c2)cc1 ; c1ccc(-c2ccc(-c3ccc(N(c4ccccc4)c4ccc5c6ccccc6c6c(ccc7oc8ccccc8c76)c5c4)cc3)cc2)cc1]

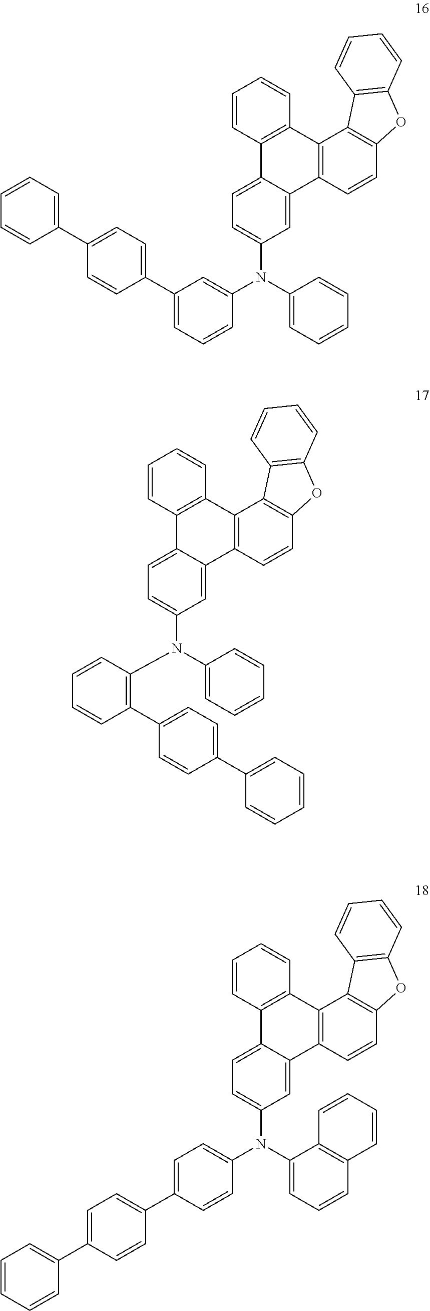 [SMILES: c1ccc(-c2ccc(-c3cccc(N(c4ccccc4)c4ccc5c6ccccc6c6c(ccc7oc8ccccc8c76)c5c4)c3)cc2)cc1 ; c1ccc(-c2ccc(-c3ccccc3N(c3ccccc3)c3ccc4c5ccccc5c5c(ccc6oc7ccccc7c65)c4c3)cc2)cc1 ; c1ccc(-c2ccc(-c3ccc(N(c4ccc5c6ccccc6c6c(ccc7oc8ccccc8c76)c5c4)c4cccc5ccccc45)cc3)cc2)cc1]

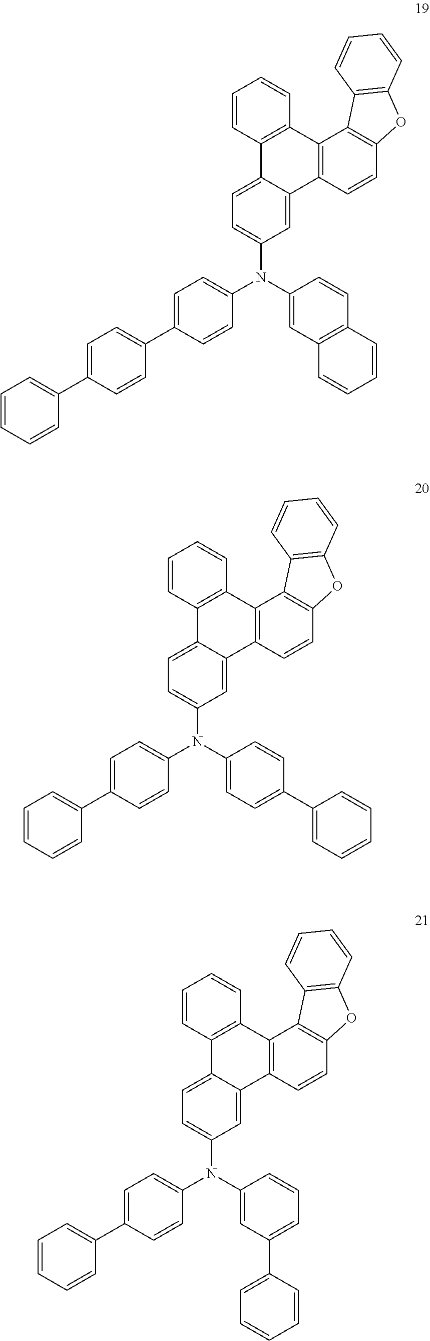 [SMILES: c1ccc(-c2ccc(-c3ccc(N(c4ccc5ccccc5c4)c4ccc5c6ccccc6c6c(ccc7oc8ccccc8c76)c5c4)cc3)cc2)cc1 ; c1ccc(-c2ccc(N(c3ccc(-c4ccccc4)cc3)c3ccc4c5ccccc5c5c(ccc6oc7ccccc7c65)c4c3)cc2)cc1 ; c1ccc(-c2ccc(N(c3cccc(-c4ccccc4)c3)c3ccc4c5ccccc5c5c(ccc6oc7ccccc7c65)c4c3)cc2)cc1]

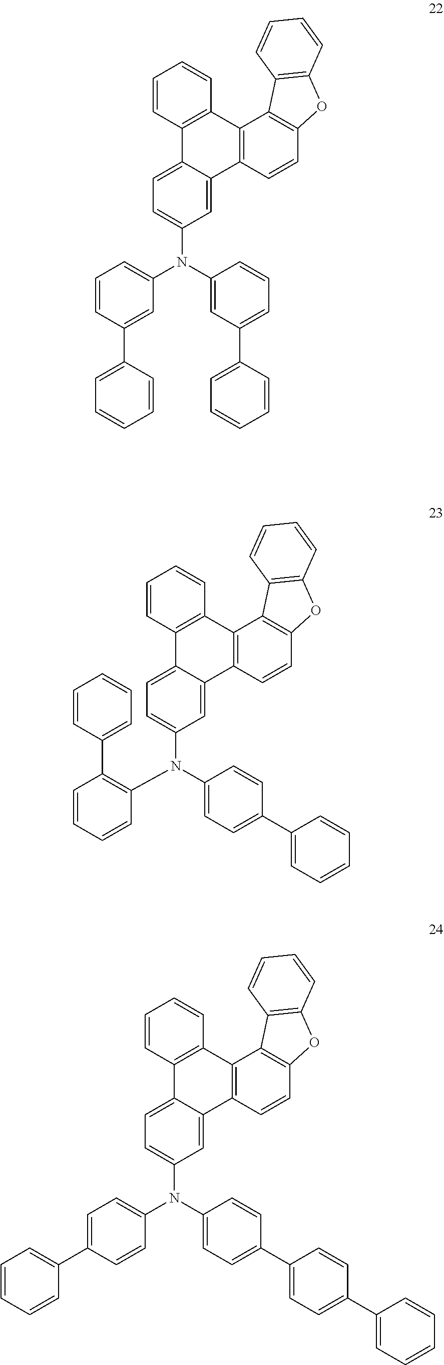 [SMILES: c1ccc(-c2cccc(N(c3cccc(-c4ccccc4)c3)c3ccc4c5ccccc5c5c(ccc6oc7ccccc7c65)c4c3)c2)cc1 ; c1ccc(-c2ccc(N(c3ccc4c5ccccc5c5c(ccc6oc7ccccc7c65)c4c3)c3ccccc3-c3ccccc3)cc2)cc1 ; c1ccc(-c2ccc(-c3ccc(N(c4ccc(-c5ccccc5)cc4)c4ccc5c6ccccc6c6c(ccc7oc8ccccc8c76)c5c4)cc3)cc2)cc1]

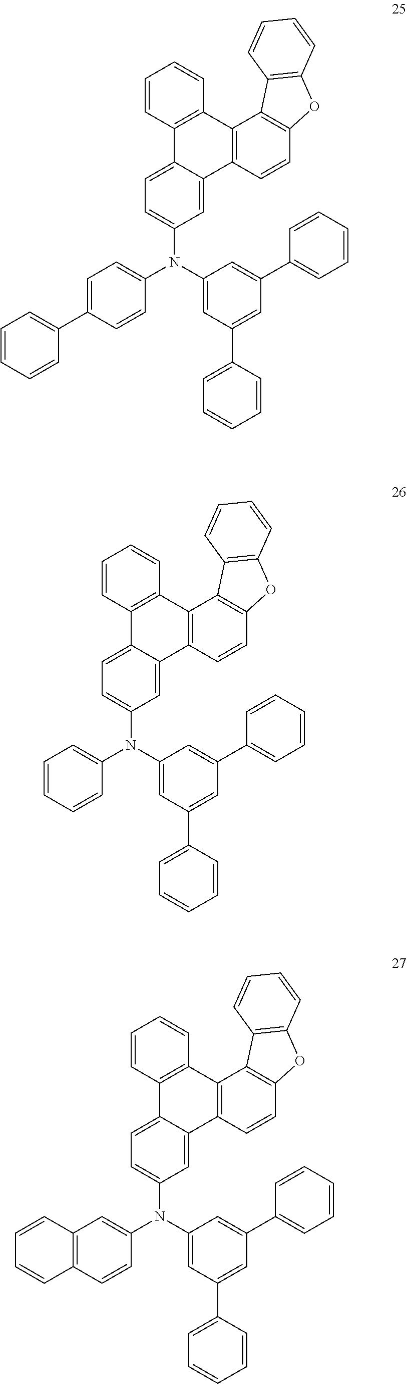 [SMILES: c1ccc(-c2ccc(N(c3cc(-c4ccccc4)cc(-c4ccccc4)c3)c3ccc4c5ccccc5c5c(ccc6oc7ccccc7c65)c4c3)cc2)cc1 ; c1ccc(-c2cc(-c3ccccc3)cc(N(c3ccccc3)c3ccc4c5ccccc5c5c(ccc6oc7ccccc7c65)c4c3)c2)cc1 ; c1ccc(-c2cc(-c3ccccc3)cc(N(c3ccc4ccccc4c3)c3ccc4c5ccccc5c5c(ccc6oc7ccccc7c65)c4c3)c2)cc1]

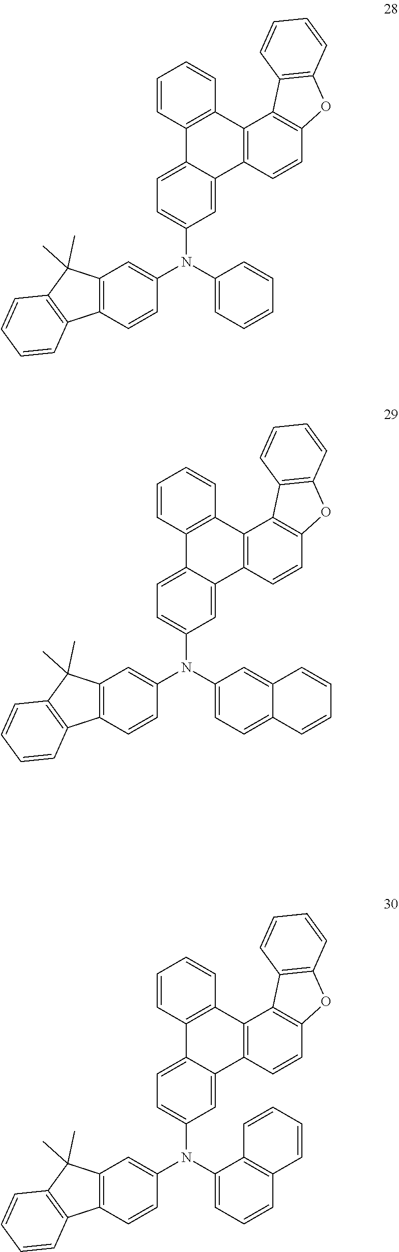 [SMILES: CC1(C)c2ccccc2-c2ccc(N(c3ccccc3)c3ccc4c5ccccc5c5c(ccc6oc7ccccc7c65)c4c3)cc21 ; CC1(C)c2ccccc2-c2ccc(N(c3ccc4ccccc4c3)c3ccc4c5ccccc5c5c(ccc6oc7ccccc7c65)c4c3)cc21 ; CC1(C)c2ccccc2-c2ccc(N(c3ccc4c5ccccc5c5c(ccc6oc7ccccc7c65)c4c3)c3cccc4ccccc34)cc21]

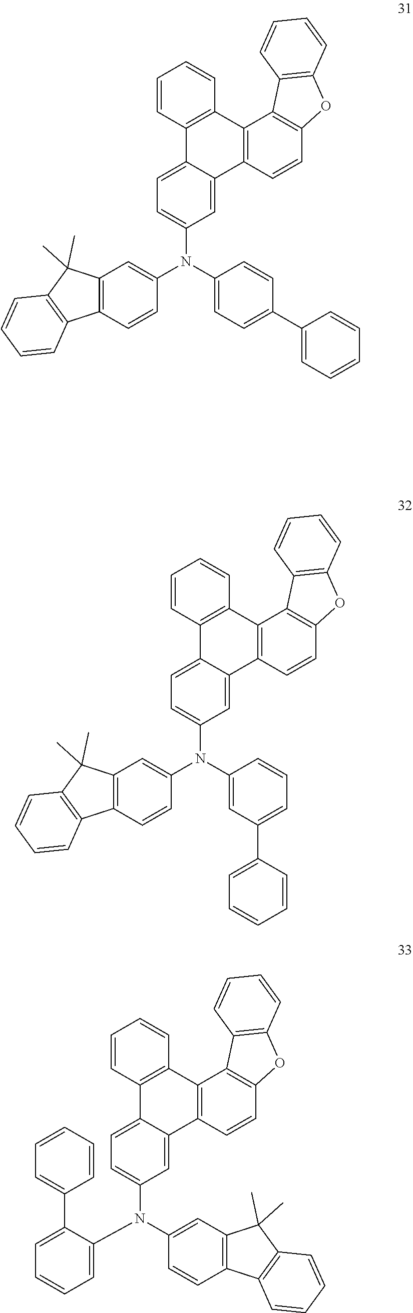 [SMILES: CC1(C)c2ccccc2-c2ccc(N(c3ccc(-c4ccccc4)cc3)c3ccc4c5ccccc5c5c(ccc6oc7ccccc7c65)c4c3)cc21 ; CC1(C)c2ccccc2-c2ccc(N(c3cccc(-c4ccccc4)c3)c3ccc4c5ccccc5c5c(ccc6oc7ccccc7c65)c4c3)cc21 ; CC1(C)c2ccccc2-c2ccc(N(c3ccc4c5ccccc5c5c(ccc6oc7ccccc7c65)c4c3)c3ccccc3-c3ccccc3)cc21]

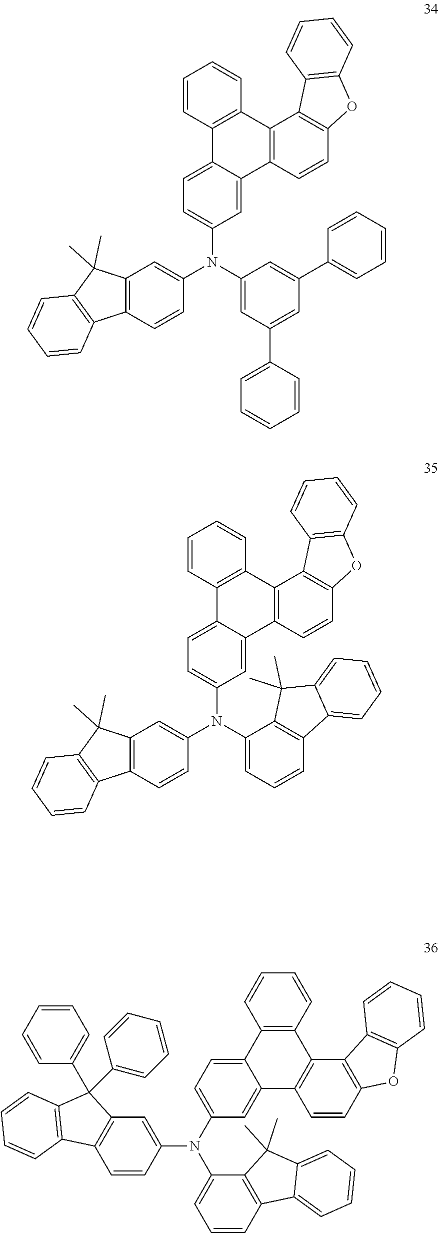 [SMILES: CC1(C)c2ccccc2-c2ccc(N(c3cc(-c4ccccc4)cc(-c4ccccc4)c3)c3ccc4c5ccccc5c5c(ccc6oc7ccccc7c65)c4c3)cc21 ; CC1(C)c2ccccc2-c2ccc(N(c3ccc4c5ccccc5c5c(ccc6oc7ccccc7c65)c4c3)c3cccc4c3C(C)(C)c3ccccc3-4)cc21 ; CC1(C)c2ccccc2-c2cccc(N(c3ccc4c(c3)C(c3ccccc3)(c3ccccc3)c3ccccc3-4)c3ccc4c5ccccc5c5c(ccc6oc7ccccc7c65)c4c3)c21]

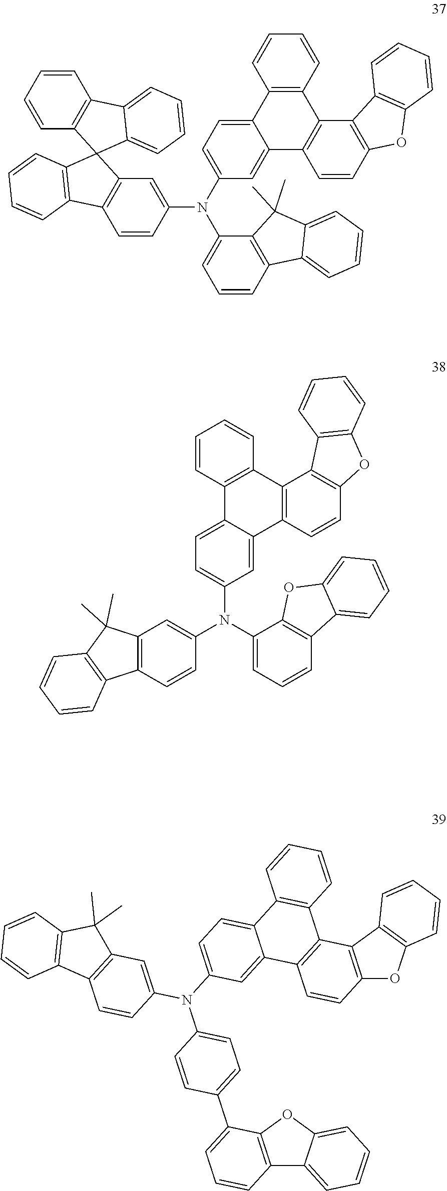 [SMILES: CC1(C)c2ccccc2-c2cccc(N(c3ccc4c(c3)C3(c5ccccc5-c5ccccc53)c3ccccc3-4)c3ccc4c5ccccc5c5c(ccc6oc7ccccc7c65)c4c3)c21 ; CC1(C)c2ccccc2-c2ccc(N(c3ccc4c5ccccc5c5c(ccc6oc7ccccc7c65)c4c3)c3cccc4c3oc3ccccc34)cc21 ; CC1(C)c2ccccc2-c2ccc(N(c3ccc(-c4cccc5c4oc4ccccc45)cc3)c3ccc4c5ccccc5c5c(ccc6oc7ccccc7c65)c4c3)cc21]

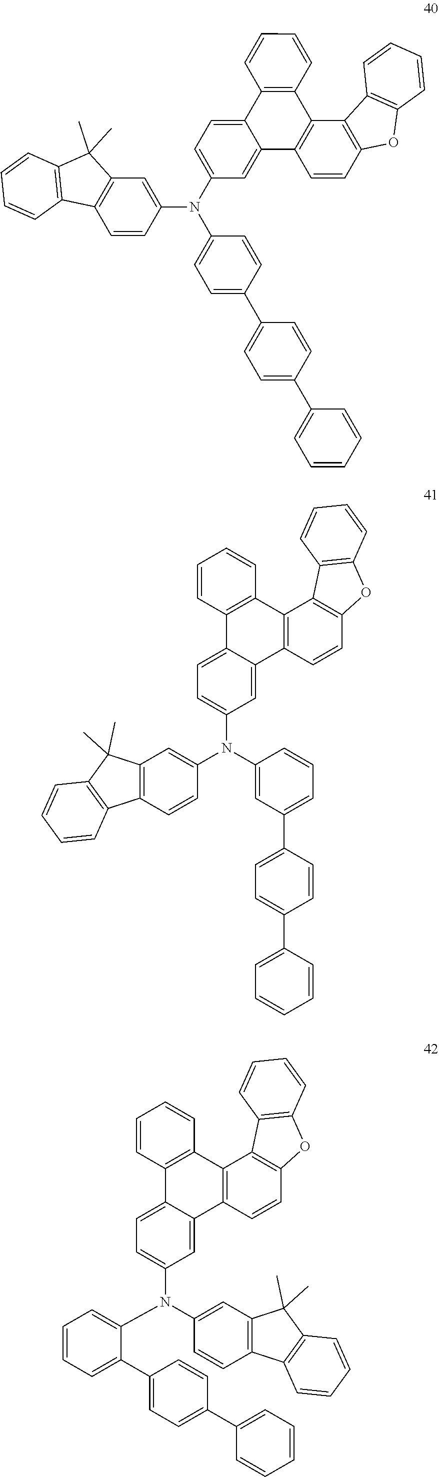

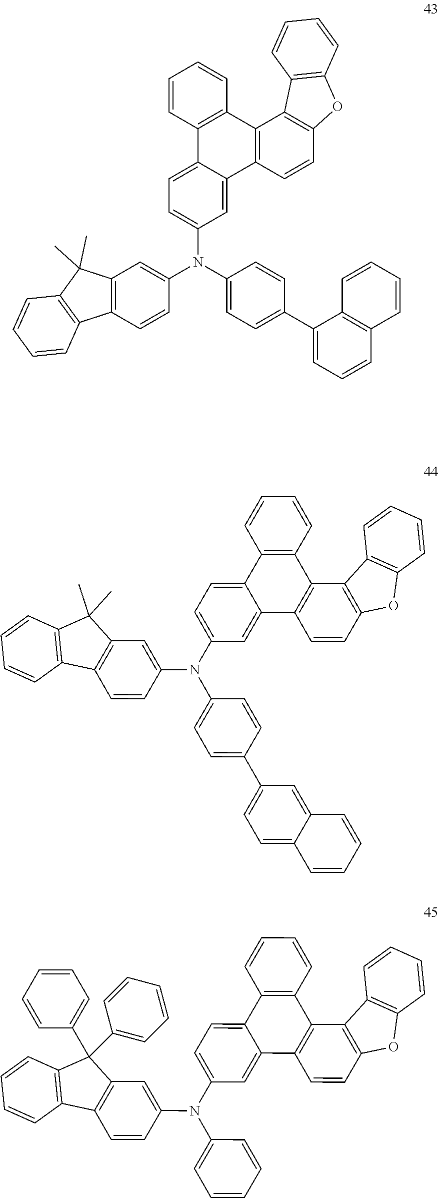 [SMILES: CC1(C)c2ccccc2-c2ccc(N(c3ccc(-c4cccc5ccccc45)cc3)c3ccc4c5ccccc5c5c(ccc6oc7ccccc7c65)c4c3)cc21 ; CC1(C)c2ccccc2-c2ccc(N(c3ccc(-c4ccc5ccccc5c4)cc3)c3ccc4c5ccccc5c5c(ccc6oc7ccccc7c65)c4c3)cc21 ; c1ccc(N(c2ccc3c(c2)C(c2ccccc2)(c2ccccc2)c2ccccc2-3)c2ccc3c4ccccc4c4c(ccc5oc6ccccc6c54)c3c2)cc1]

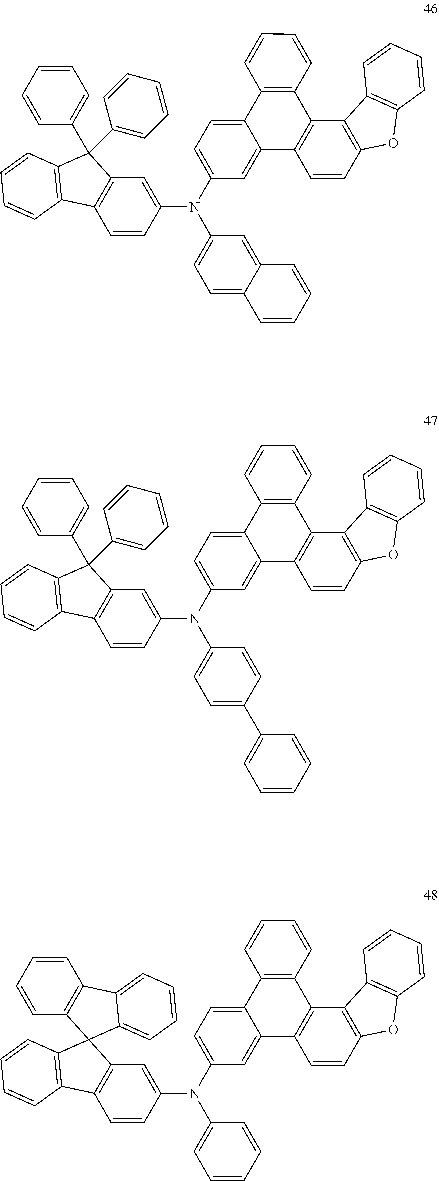 [SMILES: c1ccc(C2(c3ccccc3)c3ccccc3-c3ccc(N(c4ccc5ccccc5c4)c4ccc5c6ccccc6c6c(ccc7oc8ccccc8c76)c5c4)cc32)cc1 ; c1ccc(-c2ccc(N(c3ccc4c(c3)C(c3ccccc3)(c3ccccc3)c3ccccc3-4)c3ccc4c5ccccc5c5c(ccc6oc7ccccc7c65)c4c3)cc2)cc1 ; c1ccc(N(c2ccc3c(c2)C2(c4ccccc4-c4ccccc42)c2ccccc2-3)c2ccc3c4ccccc4c4c(ccc5oc6ccccc6c54)c3c2)cc1]

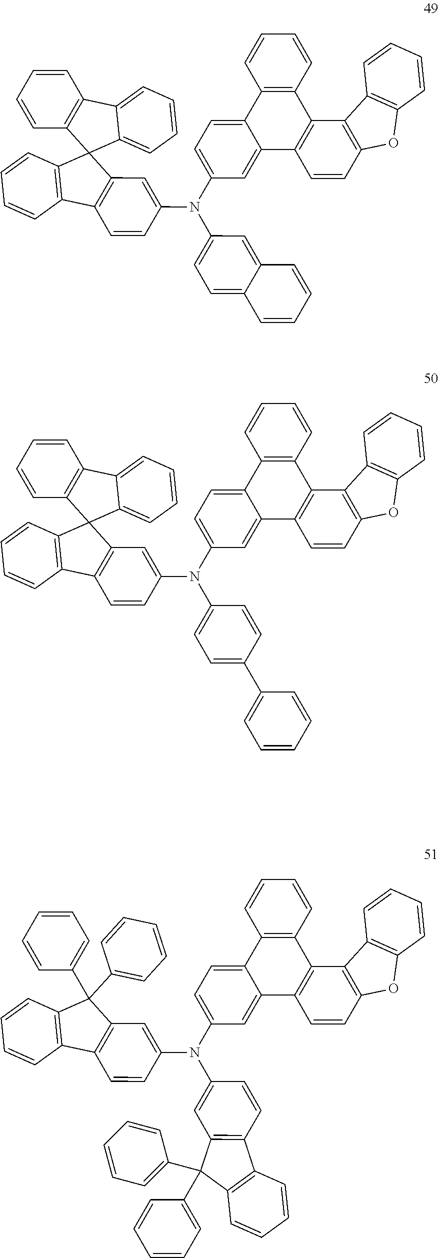 [SMILES: c1ccc2c(c1)-c1ccccc1C21c2ccccc2-c2ccc(N(c3ccc4ccccc4c3)c3ccc4c5ccccc5c5c(ccc6oc7ccccc7c65)c4c3)cc21 ; c1ccc(-c2ccc(N(c3ccc4c(c3)C3(c5ccccc5-c5ccccc53)c3ccccc3-4)c3ccc4c5ccccc5c5c(ccc6oc7ccccc7c65)c4c3)cc2)cc1 ; c1ccc(C2(c3ccccc3)c3ccccc3-c3ccc(N(c4ccc5c(c4)C(c4ccccc4)(c4ccccc4)c4ccccc4-5)c4ccc5c6ccccc6c6c(ccc7oc8ccccc8c76)c5c4)cc32)cc1]

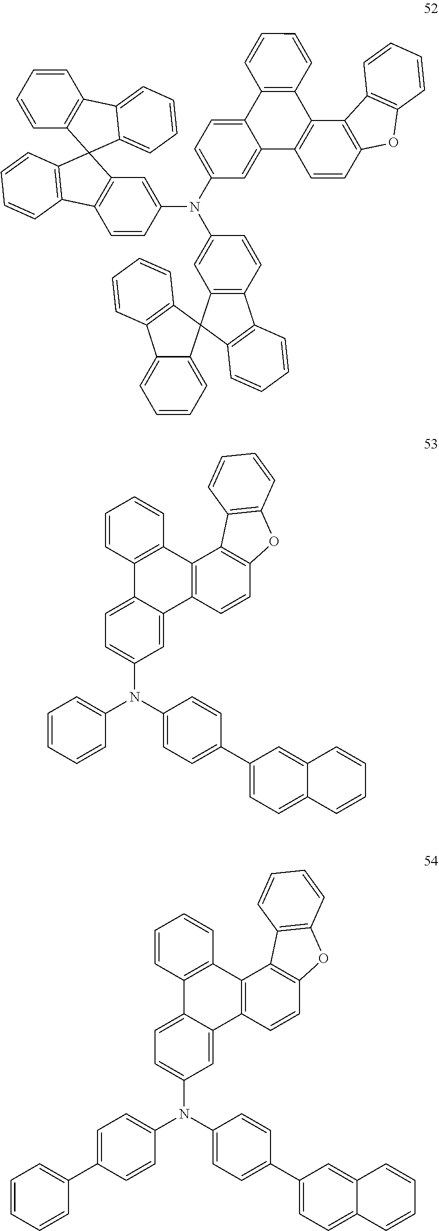 [SMILES: c1ccc2c(c1)-c1ccccc1C21c2ccccc2-c2ccc(N(c3ccc4c(c3)C3(c5ccccc5-c5ccccc53)c3ccccc3-4)c3ccc4c5ccccc5c5c(ccc6oc7ccccc7c65)c4c3)cc21 ; c1ccc(N(c2ccc(-c3ccc4ccccc4c3)cc2)c2ccc3c4ccccc4c4c(ccc5oc6ccccc6c54)c3c2)cc1 ; c1ccc(-c2ccc(N(c3ccc(-c4ccc5ccccc5c4)cc3)c3ccc4c5ccccc5c5c(ccc6oc7ccccc7c65)c4c3)cc2)cc1]

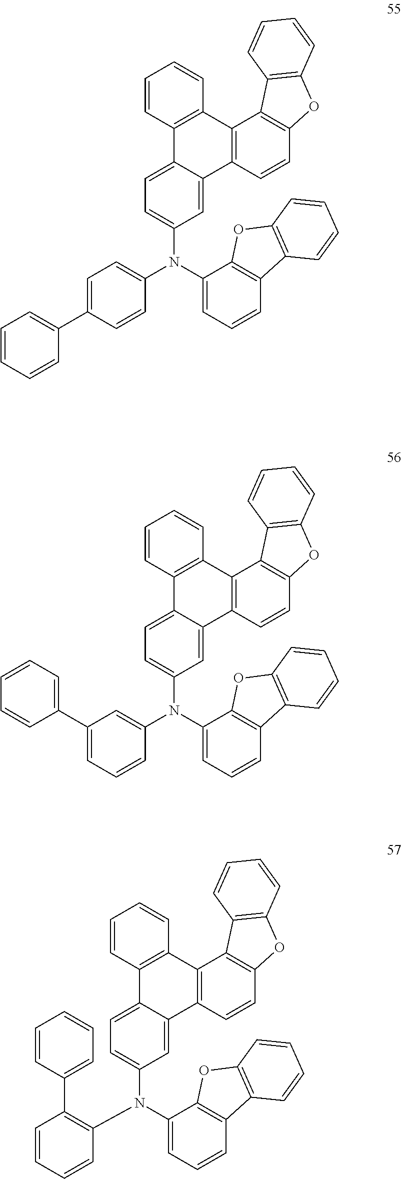 [SMILES: c1ccc(-c2ccc(N(c3ccc4c5ccccc5c5c(ccc6oc7ccccc7c65)c4c3)c3cccc4c3oc3ccccc34)cc2)cc1 ; c1ccc(-c2cccc(N(c3ccc4c5ccccc5c5c(ccc6oc7ccccc7c65)c4c3)c3cccc4c3oc3ccccc34)c2)cc1 ; c1ccc(-c2ccccc2N(c2ccc3c4ccccc4c4c(ccc5oc6ccccc6c54)c3c2)c2cccc3c2oc2ccccc23)cc1]

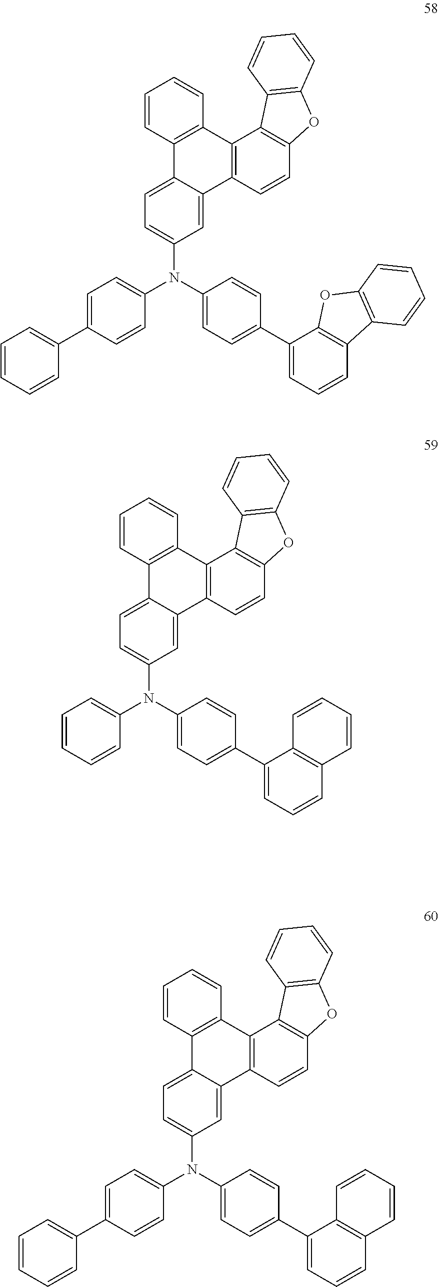 [SMILES: c1ccc(-c2ccc(N(c3ccc(-c4cccc5c4oc4ccccc45)cc3)c3ccc4c5ccccc5c5c(ccc6oc7ccccc7c65)c4c3)cc2)cc1 ; c1ccc(N(c2ccc(-c3cccc4ccccc34)cc2)c2ccc3c4ccccc4c4c(ccc5oc6ccccc6c54)c3c2)cc1 ; c1ccc(-c2ccc(N(c3ccc(-c4cccc5ccccc45)cc3)c3ccc4c5ccccc5c5c(ccc6oc7ccccc7c65)c4c3)cc2)cc1]

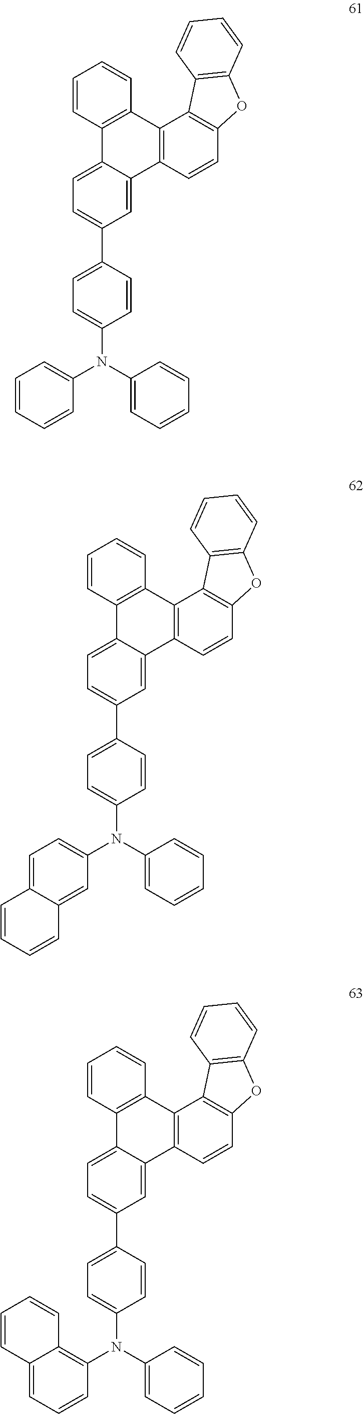 [SMILES: c1ccc(N(c2ccccc2)c2ccc(-c3ccc4c5ccccc5c5c(ccc6oc7ccccc7c65)c4c3)cc2)cc1 ; c1ccc(N(c2ccc(-c3ccc4c5ccccc5c5c(ccc6oc7ccccc7c65)c4c3)cc2)c2ccc3ccccc3c2)cc1 ; c1ccc(N(c2ccc(-c3ccc4c5ccccc5c5c(ccc6oc7ccccc7c65)c4c3)cc2)c2cccc3ccccc23)cc1]

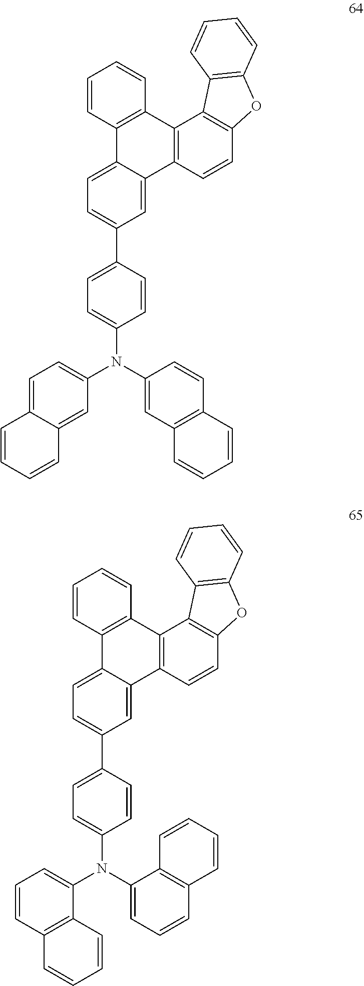 [SMILES: c1ccc2cc(N(c3ccc(-c4ccc5c6ccccc6c6c(ccc7oc8ccccc8c76)c5c4)cc3)c3ccc4ccccc4c3)ccc2c1 ; c1ccc2c(N(c3ccc(-c4ccc5c6ccccc6c6c(ccc7oc8ccccc8c76)c5c4)cc3)c3cccc4ccccc34)cccc2c1]

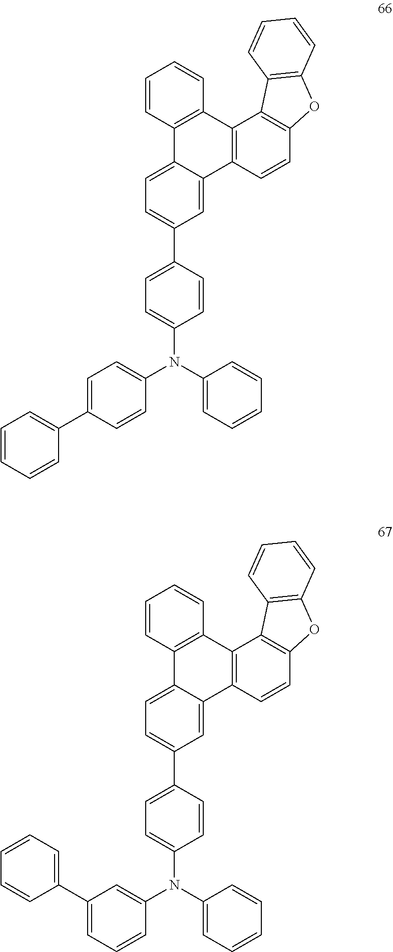 [SMILES: c1ccc(-c2ccc(N(c3ccccc3)c3ccc(-c4ccc5c6ccccc6c6c(ccc7oc8ccccc8c76)c5c4)cc3)cc2)cc1 ; c1ccc(-c2cccc(N(c3ccccc3)c3ccc(-c4ccc5c6ccccc6c6c(ccc7oc8ccccc8c76)c5c4)cc3)c2)cc1]

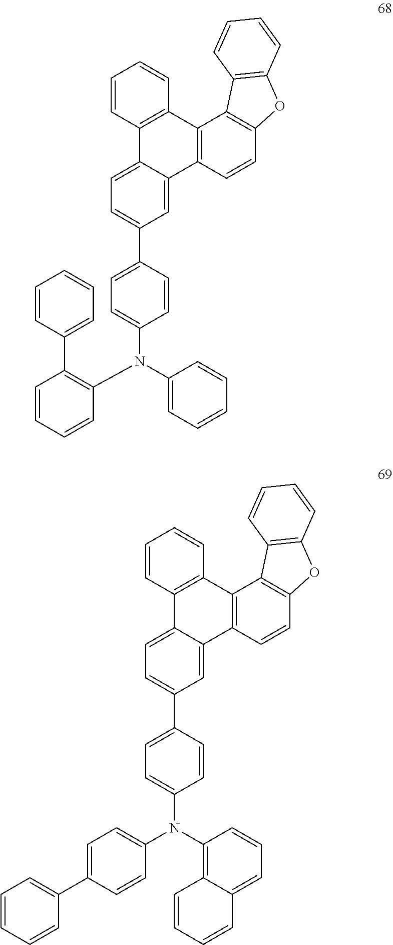 [SMILES: c1ccc(-c2ccccc2N(c2ccccc2)c2ccc(-c3ccc4c5ccccc5c5c(ccc6oc7ccccc7c65)c4c3)cc2)cc1 ; c1ccc(-c2ccc(N(c3ccc(-c4ccc5c6ccccc6c6c(ccc7oc8ccccc8c76)c5c4)cc3)c3cccc4ccccc34)cc2)cc1]

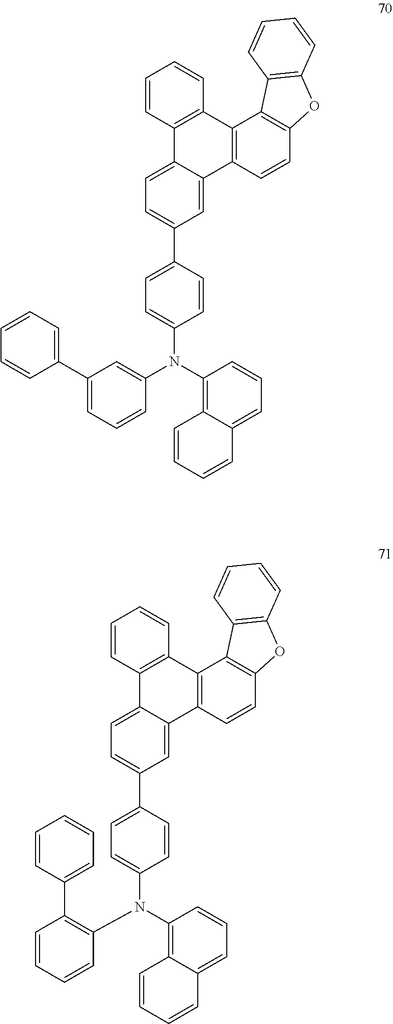 [SMILES: c1ccc(-c2cccc(N(c3ccc(-c4ccc5c6ccccc6c6c(ccc7oc8ccccc8c76)c5c4)cc3)c3cccc4ccccc34)c2)cc1 ; c1ccc(-c2ccccc2N(c2ccc(-c3ccc4c5ccccc5c5c(ccc6oc7ccccc7c65)c4c3)cc2)c2cccc3ccccc23)cc1]

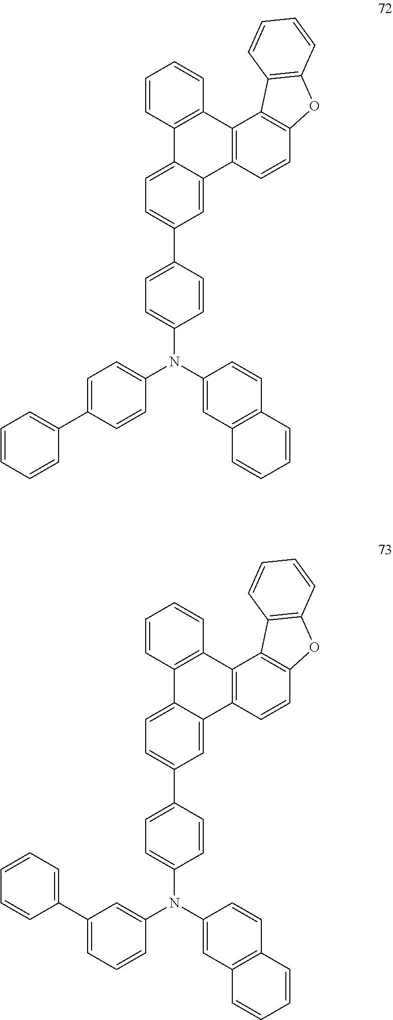 [SMILES: c1ccc(-c2ccc(N(c3ccc(-c4ccc5c6ccccc6c6c(ccc7oc8ccccc8c76)c5c4)cc3)c3ccc4ccccc4c3)cc2)cc1 ; c1ccc(-c2cccc(N(c3ccc(-c4ccc5c6ccccc6c6c(ccc7oc8ccccc8c76)c5c4)cc3)c3ccc4ccccc4c3)c2)cc1]

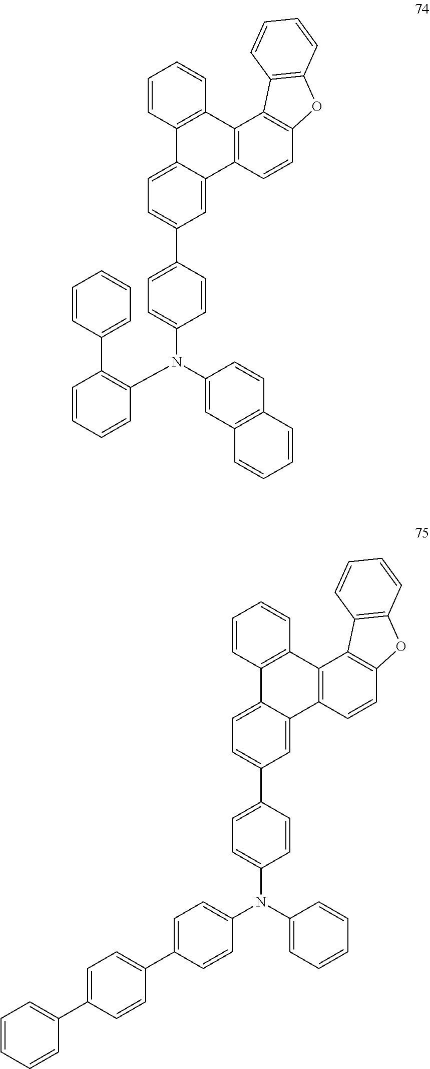 [SMILES: c1ccc(-c2ccccc2N(c2ccc(-c3ccc4c5ccccc5c5c(ccc6oc7ccccc7c65)c4c3)cc2)c2ccc3ccccc3c2)cc1 ; c1ccc(-c2ccc(-c3ccc(N(c4ccccc4)c4ccc(-c5ccc6c7ccccc7c7c(ccc8oc9ccccc9c87)c6c5)cc4)cc3)cc2)cc1]

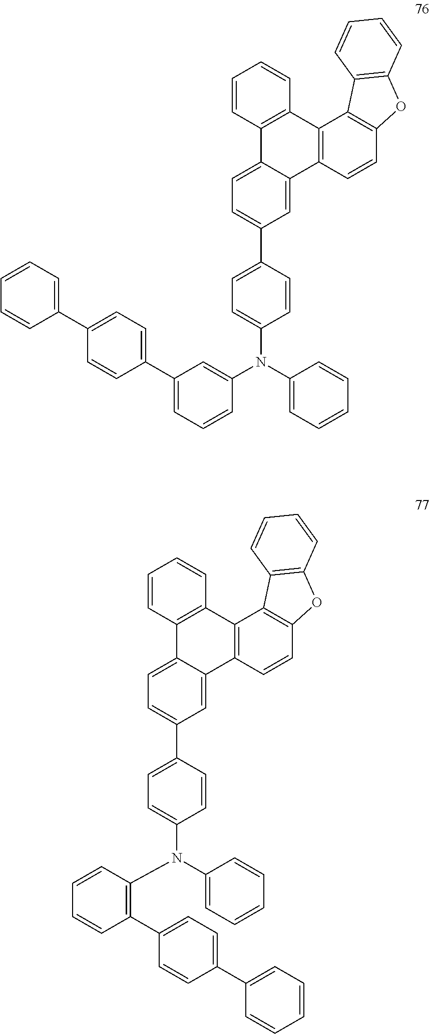 [SMILES: c1ccc(-c2ccc(-c3cccc(N(c4ccccc4)c4ccc(-c5ccc6c7ccccc7c7c(ccc8oc9ccccc9c87)c6c5)cc4)c3)cc2)cc1 ; c1ccc(-c2ccc(-c3ccccc3N(c3ccccc3)c3ccc(-c4ccc5c6ccccc6c6c(ccc7oc8ccccc8c76)c5c4)cc3)cc2)cc1]

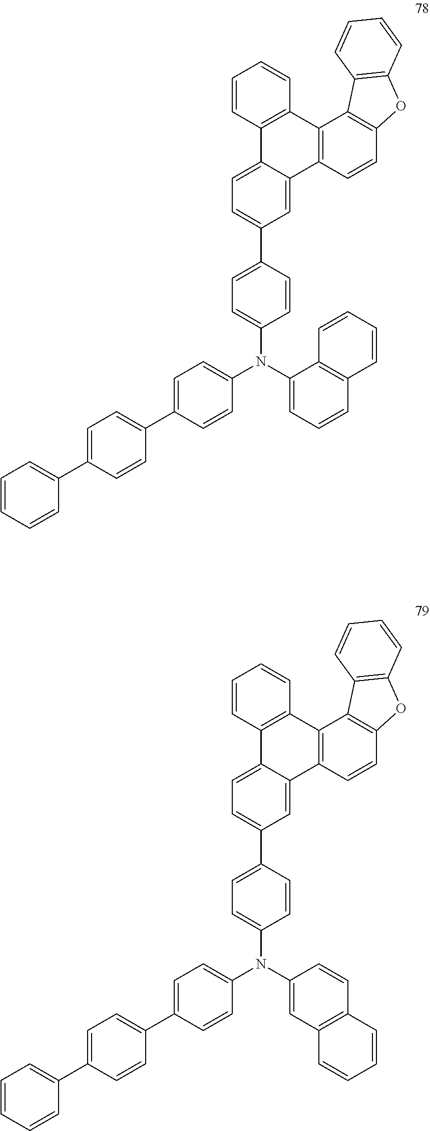 [SMILES: c1ccc(-c2ccc(-c3ccc(N(c4ccc(-c5ccc6c7ccccc7c7c(ccc8oc9ccccc9c87)c6c5)cc4)c4cccc5ccccc45)cc3)cc2)cc1 ; c1ccc(-c2ccc(-c3ccc(N(c4ccc(-c5ccc6c7ccccc7c7c(ccc8oc9ccccc9c87)c6c5)cc4)c4ccc5ccccc5c4)cc3)cc2)cc1]

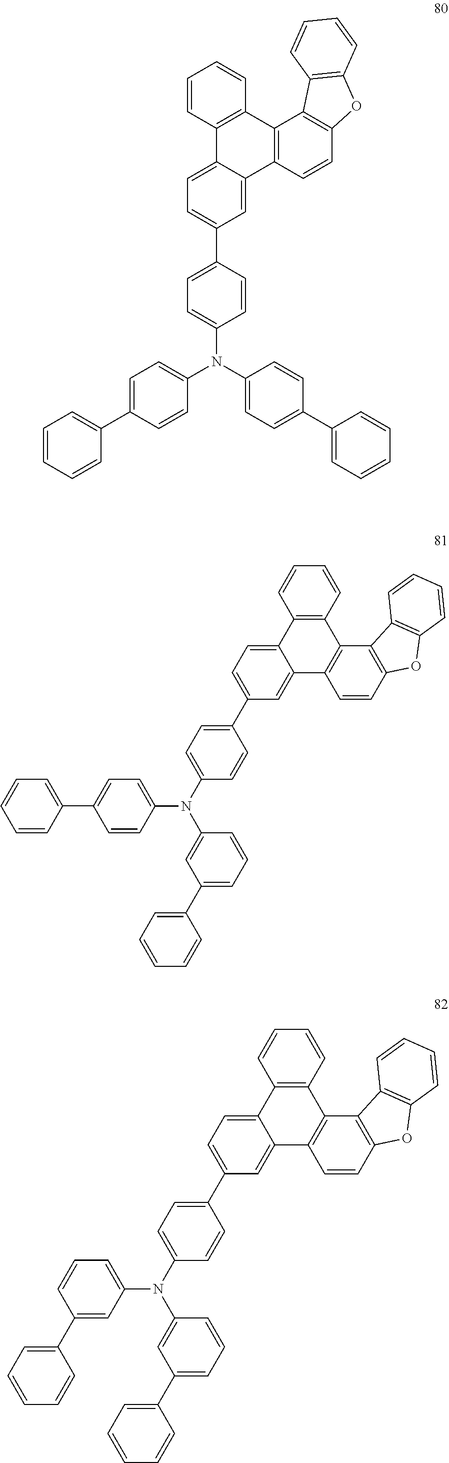 [SMILES: c1ccc(-c2ccc(N(c3ccc(-c4ccccc4)cc3)c3ccc(-c4ccc5c6ccccc6c6c(ccc7oc8ccccc8c76)c5c4)cc3)cc2)cc1 ; c1ccc(-c2ccc(N(c3ccc(-c4ccc5c6ccccc6c6c(ccc7oc8ccccc8c76)c5c4)cc3)c3cccc(-c4ccccc4)c3)cc2)cc1 ; c1ccc(-c2cccc(N(c3ccc(-c4ccc5c6ccccc6c6c(ccc7oc8ccccc8c76)c5c4)cc3)c3cccc(-c4ccccc4)c3)c2)cc1]

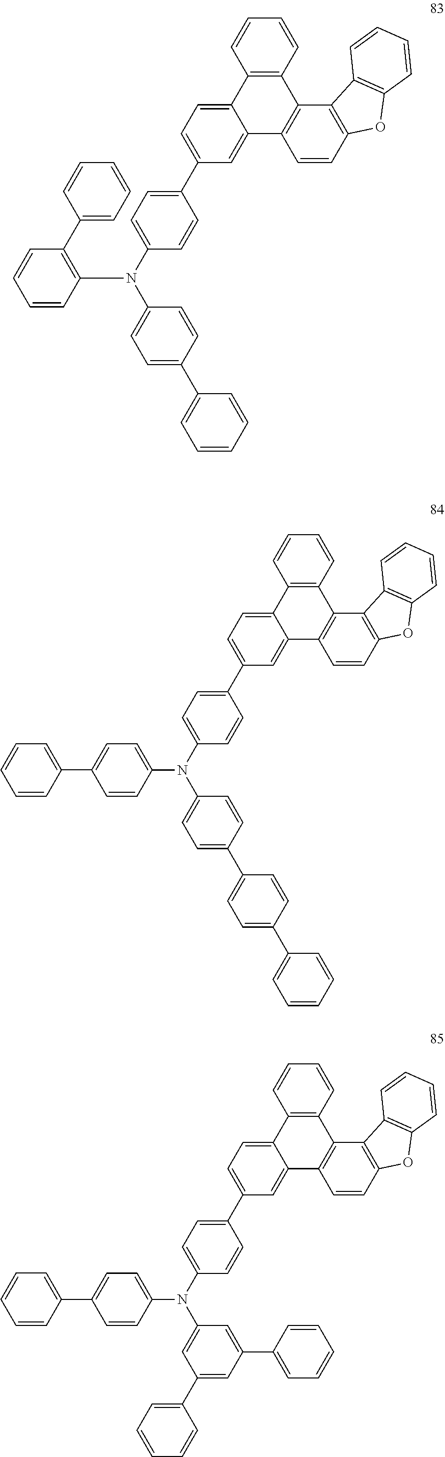 [SMILES: c1ccc(-c2ccc(N(c3ccc(-c4ccc5c6ccccc6c6c(ccc7oc8ccccc8c76)c5c4)cc3)c3ccccc3-c3ccccc3)cc2)cc1 ; c1ccc(-c2ccc(-c3ccc(N(c4ccc(-c5ccccc5)cc4)c4ccc(-c5ccc6c7ccccc7c7c(ccc8oc9ccccc9c87)c6c5)cc4)cc3)cc2)cc1 ; c1ccc(-c2ccc(N(c3ccc(-c4ccc5c6ccccc6c6c(ccc7oc8ccccc8c76)c5c4)cc3)c3cc(-c4ccccc4)cc(-c4ccccc4)c3)cc2)cc1]

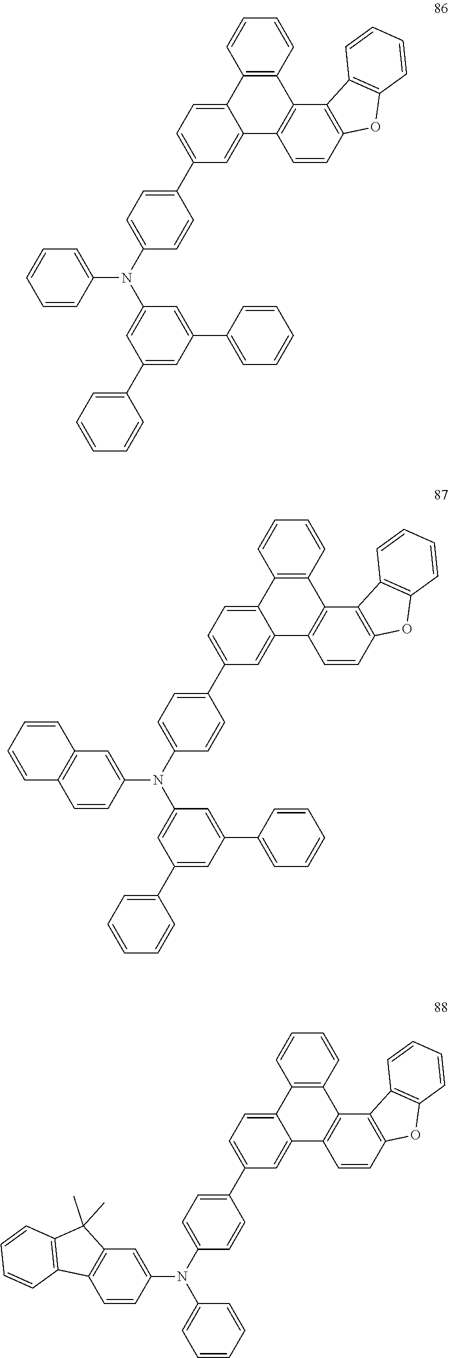 [SMILES: c1ccc(-c2cc(-c3ccccc3)cc(N(c3ccccc3)c3ccc(-c4ccc5c6ccccc6c6c(ccc7oc8ccccc8c76)c5c4)cc3)c2)cc1 ; c1ccc(-c2cc(-c3ccccc3)cc(N(c3ccc(-c4ccc5c6ccccc6c6c(ccc7oc8ccccc8c76)c5c4)cc3)c3ccc4ccccc4c3)c2)cc1 ; CC1(C)c2ccccc2-c2ccc(N(c3ccccc3)c3ccc(-c4ccc5c6ccccc6c6c(ccc7oc8ccccc8c76)c5c4)cc3)cc21]

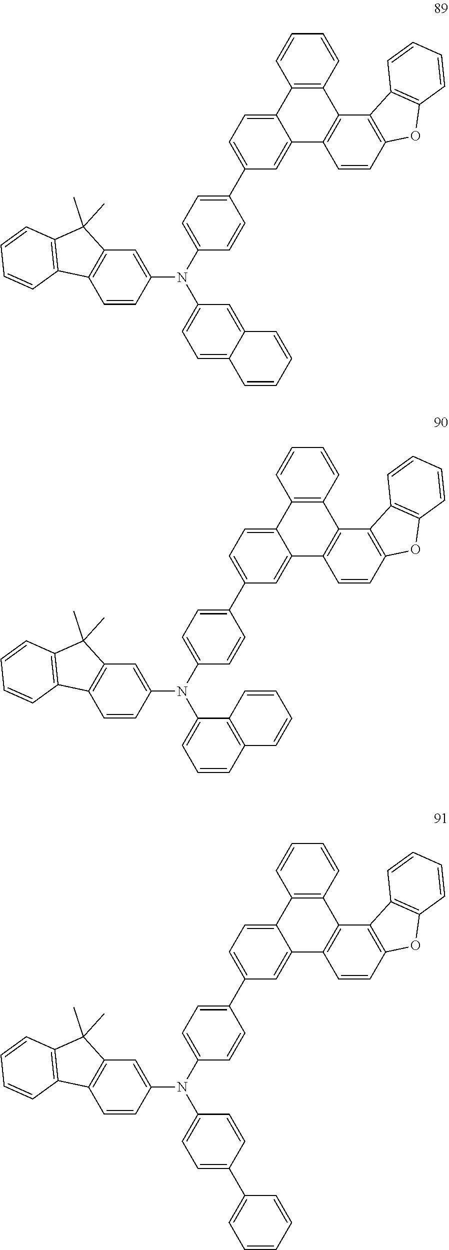 [SMILES: CC1(C)c2ccccc2-c2ccc(N(c3ccc(-c4ccc5c6ccccc6c6c(ccc7oc8ccccc8c76)c5c4)cc3)c3ccc4ccccc4c3)cc21 ; CC1(C)c2ccccc2-c2ccc(N(c3ccc(-c4ccc5c6ccccc6c6c(ccc7oc8ccccc8c76)c5c4)cc3)c3cccc4ccccc34)cc21 ; CC1(C)c2ccccc2-c2ccc(N(c3ccc(-c4ccccc4)cc3)c3ccc(-c4ccc5c6ccccc6c6c(ccc7oc8ccccc8c76)c5c4)cc3)cc21]

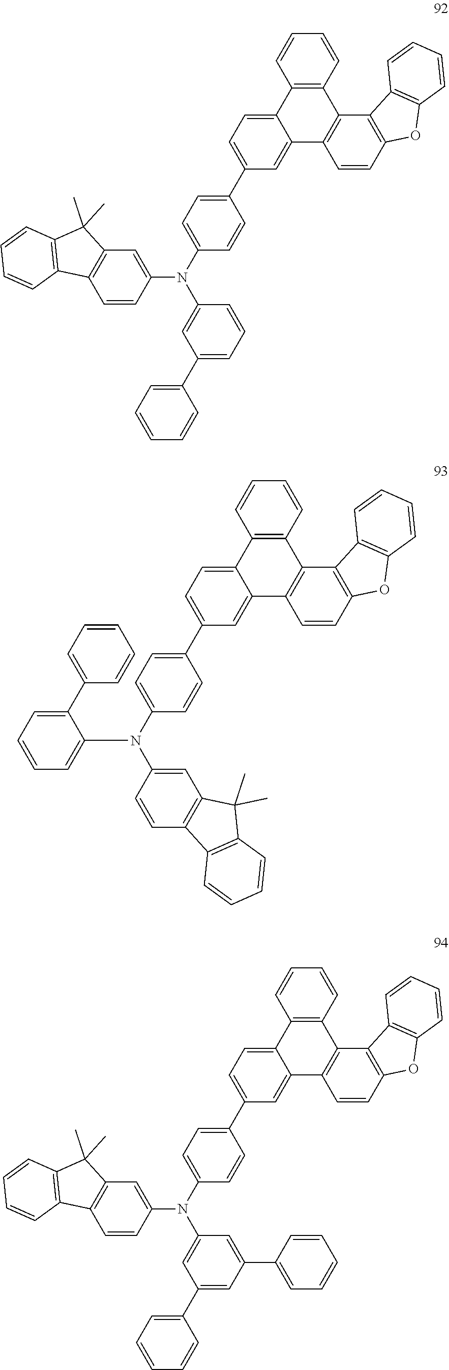 [SMILES: CC1(C)c2ccccc2-c2ccc(N(c3ccc(-c4ccc5c6ccccc6c6c(ccc7oc8ccccc8c76)c5c4)cc3)c3cccc(-c4ccccc4)c3)cc21 ; CC1(C)c2ccccc2-c2ccc(N(c3ccc(-c4ccc5c6ccccc6c6c(ccc7oc8ccccc8c76)c5c4)cc3)c3ccccc3-c3ccccc3)cc21 ; CC1(C)c2ccccc2-c2ccc(N(c3ccc(-c4ccc5c6ccccc6c6c(ccc7oc8ccccc8c76)c5c4)cc3)c3cc(-c4ccccc4)cc(-c4ccccc4)c3)cc21]

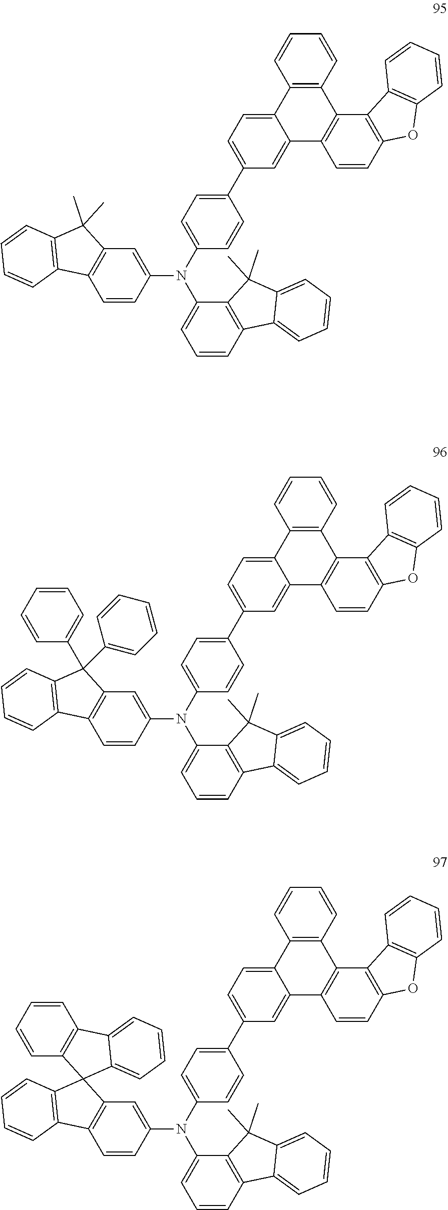 [SMILES: CC1(C)c2ccccc2-c2ccc(N(c3ccc(-c4ccc5c6ccccc6c6c(ccc7oc8ccccc8c76)c5c4)cc3)c3cccc4c3C(C)(C)c3ccccc3-4)cc21 ; CC1(C)c2ccccc2-c2cccc(N(c3ccc(-c4ccc5c6ccccc6c6c(ccc7oc8ccccc8c76)c5c4)cc3)c3ccc4c(c3)C(c3ccccc3)(c3ccccc3)c3ccccc3-4)c21 ; CC1(C)c2ccccc2-c2cccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)c21]

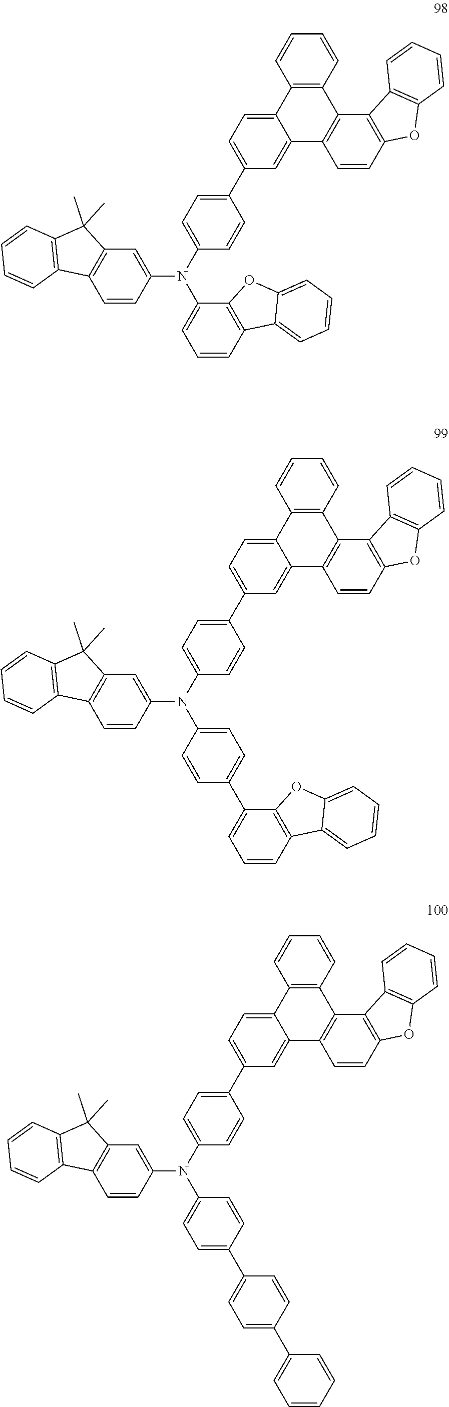 [SMILES: CC1(C)c2ccccc2-c2ccc(N(c3ccc(-c4ccc5c6ccccc6c6c(ccc7oc8ccccc8c76)c5c4)cc3)c3cccc4c3oc3ccccc34)cc21 ; CC1(C)c2ccccc2-c2ccc(N(c3ccc(-c4ccc5c6ccccc6c6c(ccc7oc8ccccc8c76)c5c4)cc3)c3ccc(-c4cccc5c4oc4ccccc45)cc3)cc21 ; CC1(C)c2ccccc2-c2ccc(N(c3ccc(-c4ccc(-c5ccccc5)cc4)cc3)c3ccc(-c4ccc5c6ccccc6c6c(ccc7oc8ccccc8c76)c5c4)cc3)cc21]

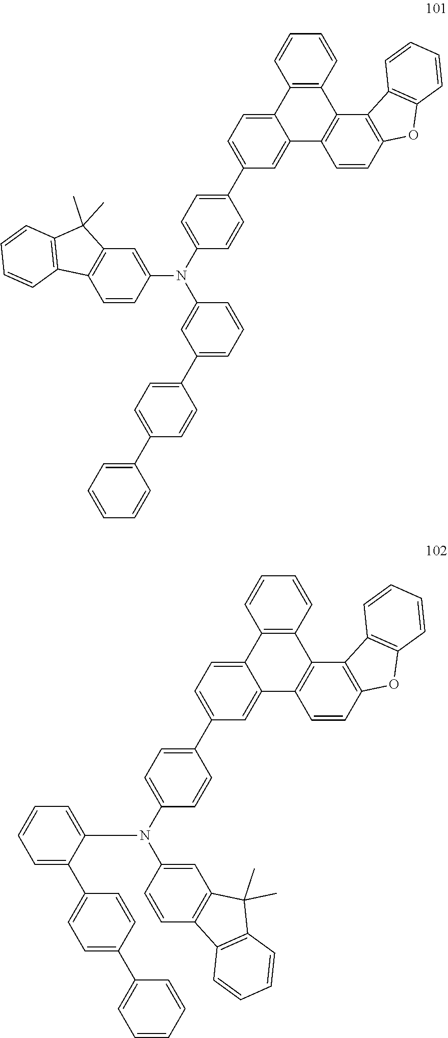 [SMILES: CC1(C)c2ccccc2-c2ccc(N(c3ccc(-c4ccc5c6ccccc6c6c(ccc7oc8ccccc8c76)c5c4)cc3)c3cccc(-c4ccc(-c5ccccc5)cc4)c3)cc21 ; CC1(C)c2ccccc2-c2ccc(N(c3ccc(-c4ccc5c6ccccc6c6c(ccc7oc8ccccc8c76)c5c4)cc3)c3ccccc3-c3ccc(-c4ccccc4)cc3)cc21]

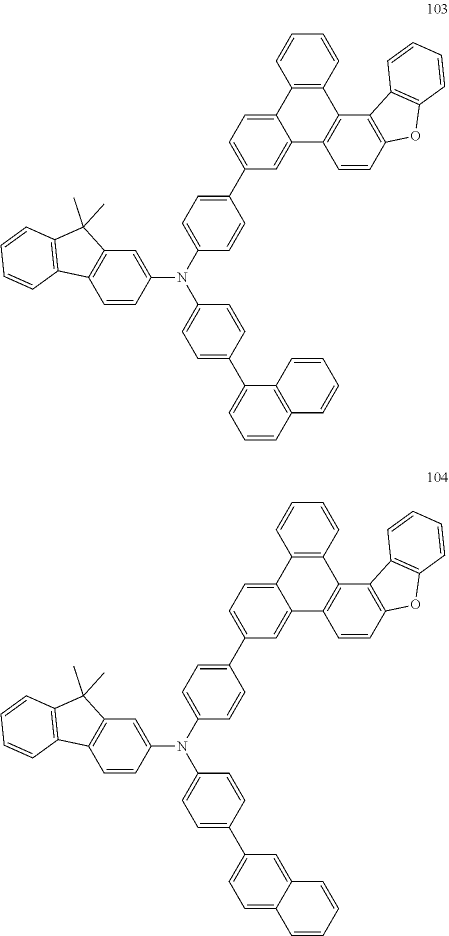 [SMILES: CC1(C)c2ccccc2-c2ccc(N(c3ccc(-c4ccc5c6ccccc6c6c(ccc7oc8ccccc8c76)c5c4)cc3)c3ccc(-c4cccc5ccccc45)cc3)cc21 ; CC1(C)c2ccccc2-c2ccc(N(c3ccc(-c4ccc5ccccc5c4)cc3)c3ccc(-c4ccc5c6ccccc6c6c(ccc7oc8ccccc8c76)c5c4)cc3)cc21]

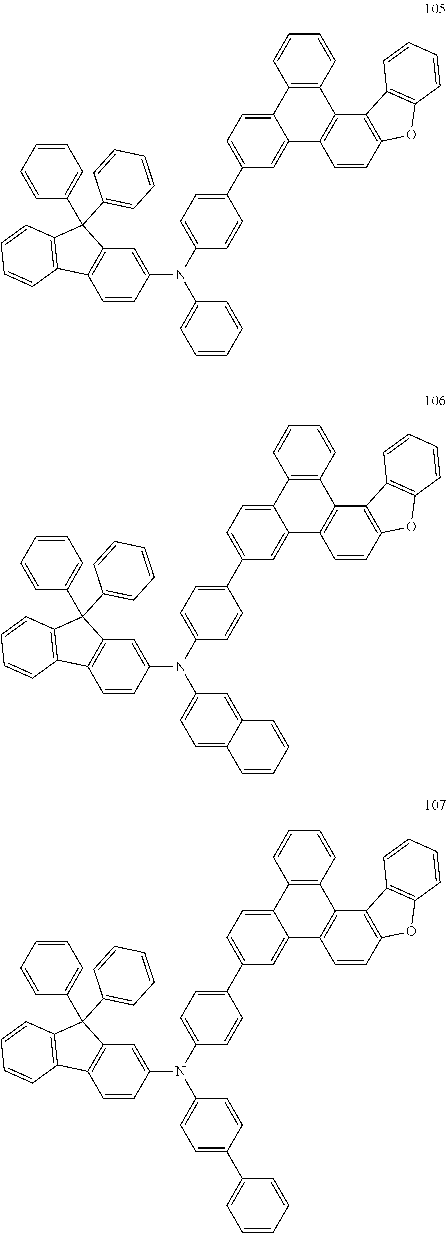 [SMILES: c1ccc(N(c2ccc(-c3ccc4c5ccccc5c5c(ccc6oc7ccccc7c65)c4c3)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccc6c7ccccc7c7c(ccc8oc9ccccc9c87)c6c5)cc4)c4ccc5ccccc5c4)cc32)cc1 ; c1ccc(-c2ccc(N(c3ccc(-c4ccc5c6ccccc6c6c(ccc7oc8ccccc8c76)c5c4)cc3)c3ccc4c(c3)C(c3ccccc3)(c3ccccc3)c3ccccc3-4)cc2)cc1]

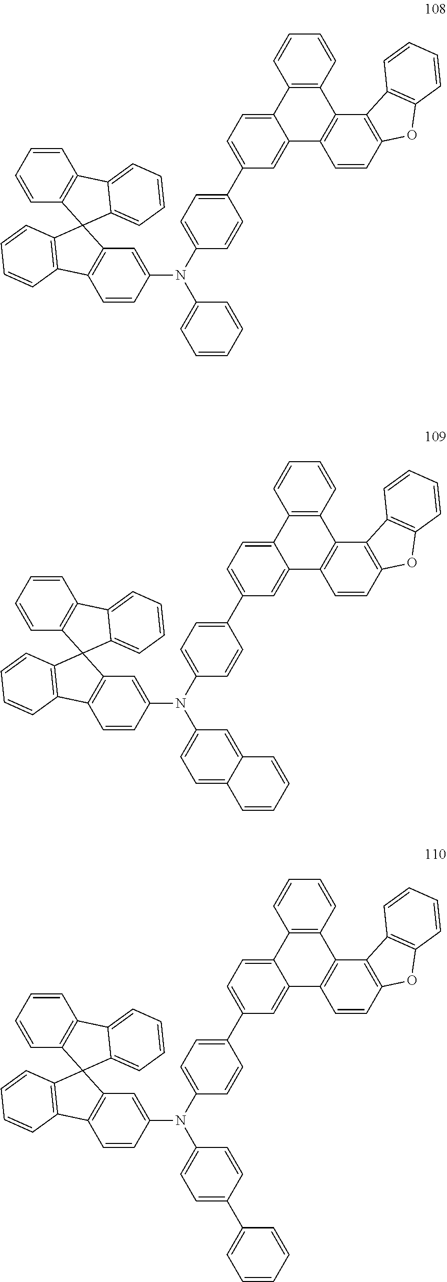 [SMILES: c1ccc(N(c2ccc(-c3ccc4c5ccccc5c5c(ccc6oc7ccccc7c65)c4c3)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4ccc5c6ccccc6c6c(ccc7oc8ccccc8c76)c5c4)cc3)c3ccc4ccccc4c3)cc21 ; c1ccc(-c2ccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)cc2)cc1]

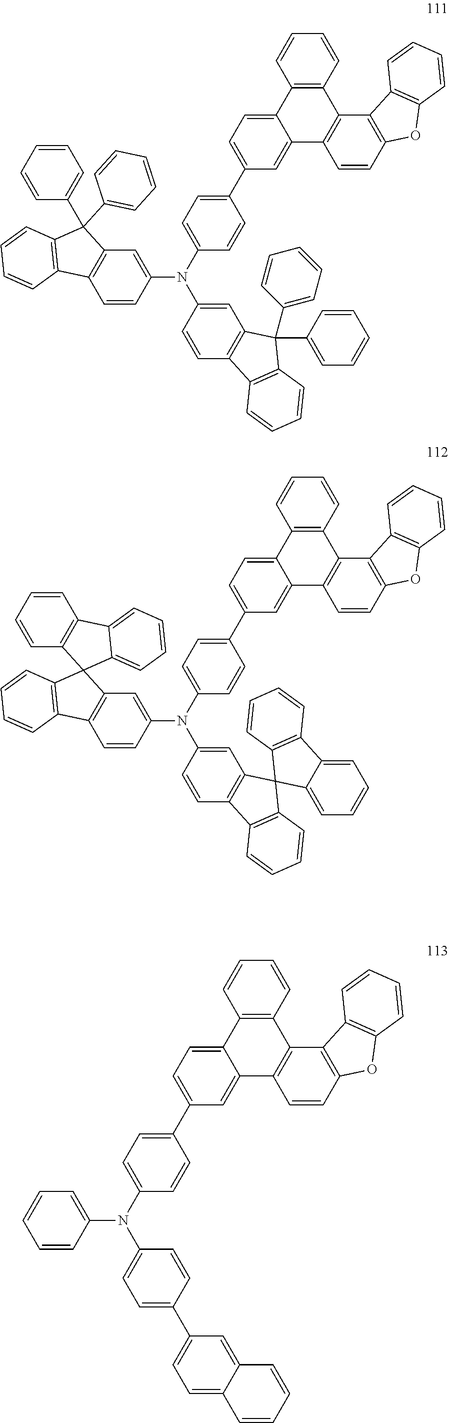 [SMILES: c1ccc(C2(c3ccccc3)c3ccccc3-c3ccc(N(c4ccc(-c5ccc6c7ccccc7c7c(ccc8oc9ccccc9c87)c6c5)cc4)c4ccc5c(c4)C(c4ccccc4)(c4ccccc4)c4ccccc4-5)cc32)cc1 ; c1ccc2c(c1)-c1ccccc1C21c2ccccc2-c2ccc(N(c3ccc(-c4ccc5c6ccccc6c6c(ccc7oc8ccccc8c76)c5c4)cc3)c3ccc4c(c3)C3(c5ccccc5-c5ccccc53)c3ccccc3-4)cc21 ; c1ccc(N(c2ccc(-c3ccc4ccccc4c3)cc2)c2ccc(-c3ccc4c5ccccc5c5c(ccc6oc7ccccc7c65)c4c3)cc2)cc1]

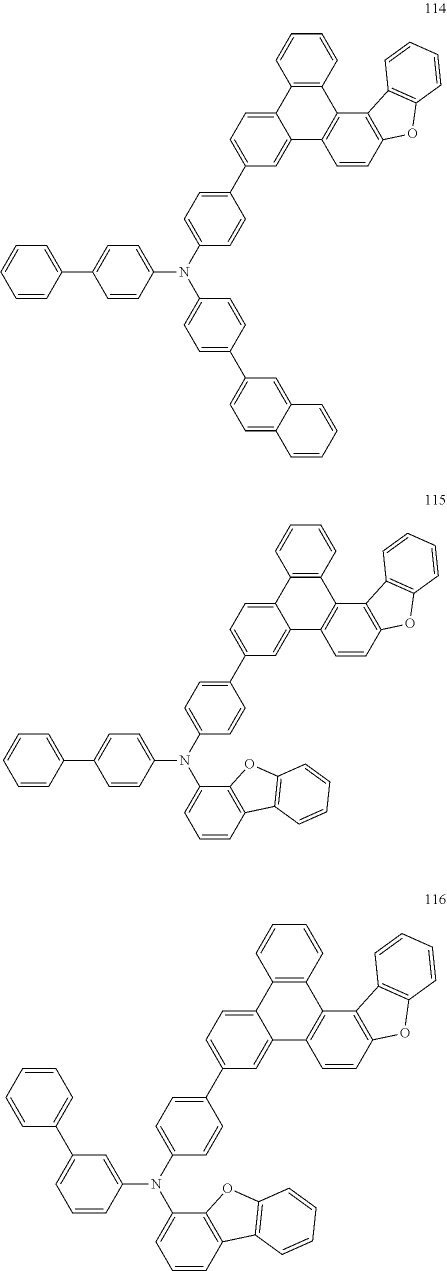 [SMILES: c1ccc(-c2ccc(N(c3ccc(-c4ccc5ccccc5c4)cc3)c3ccc(-c4ccc5c6ccccc6c6c(ccc7oc8ccccc8c76)c5c4)cc3)cc2)cc1 ; c1ccc(-c2ccc(N(c3ccc(-c4ccc5c6ccccc6c6c(ccc7oc8ccccc8c76)c5c4)cc3)c3cccc4c3oc3ccccc34)cc2)cc1 ; c1ccc(-c2cccc(N(c3ccc(-c4ccc5c6ccccc6c6c(ccc7oc8ccccc8c76)c5c4)cc3)c3cccc4c3oc3ccccc34)c2)cc1]

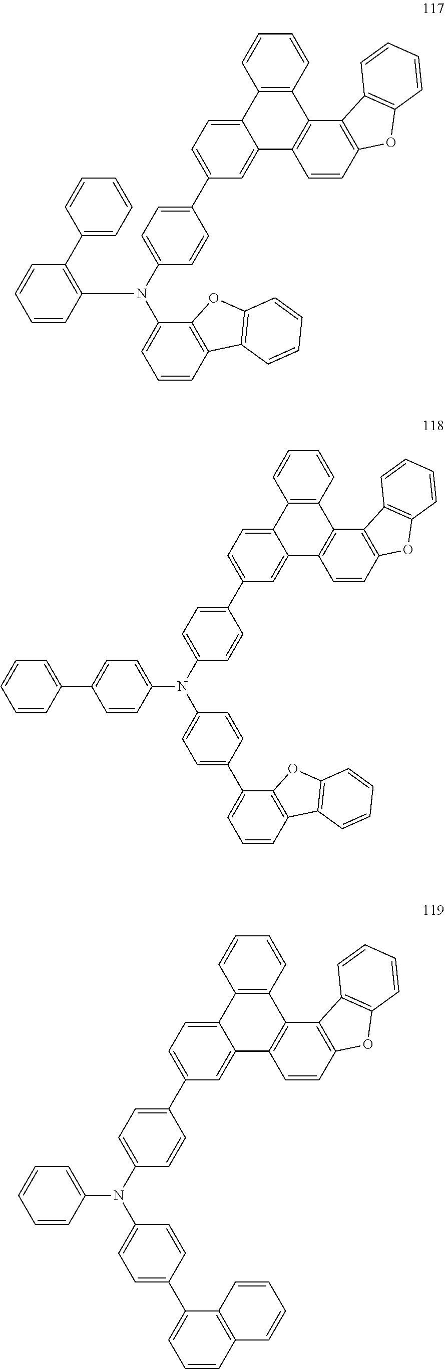 [SMILES: c1ccc(-c2ccccc2N(c2ccc(-c3ccc4c5ccccc5c5c(ccc6oc7ccccc7c65)c4c3)cc2)c2cccc3c2oc2ccccc23)cc1 ; c1ccc(-c2ccc(N(c3ccc(-c4ccc5c6ccccc6c6c(ccc7oc8ccccc8c76)c5c4)cc3)c3ccc(-c4cccc5c4oc4ccccc45)cc3)cc2)cc1 ; c1ccc(N(c2ccc(-c3ccc4c5ccccc5c5c(ccc6oc7ccccc7c65)c4c3)cc2)c2ccc(-c3cccc4ccccc34)cc2)cc1]

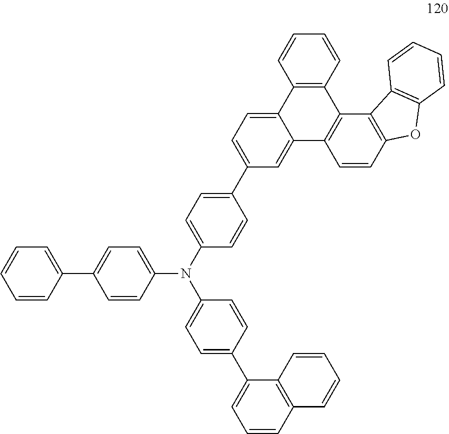 [SMILES: c1ccc(-c2ccc(N(c3ccc(-c4ccc5c6ccccc6c6c(ccc7oc8ccccc8c76)c5c4)cc3)c3ccc(-c4cccc5ccccc45)cc3)cc2)cc1]

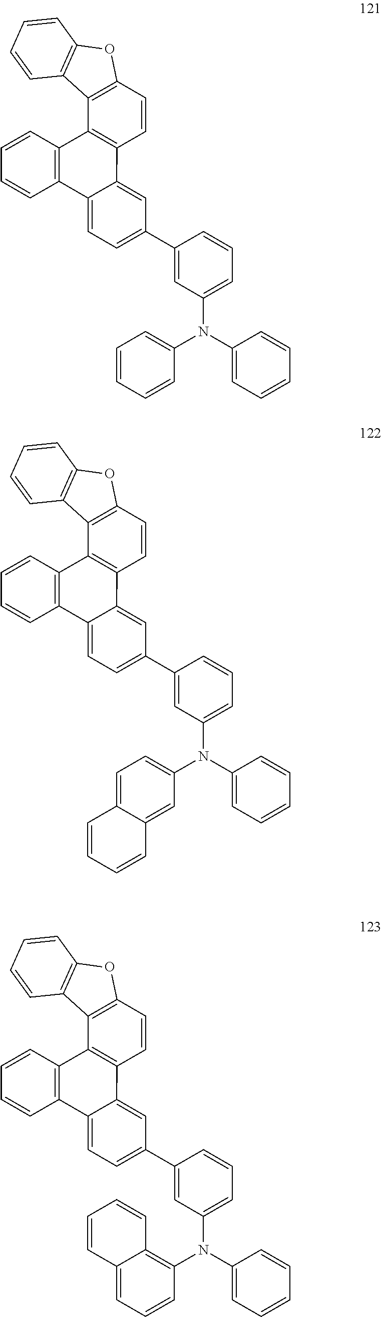 [SMILES: c1ccc(N(c2ccccc2)c2cccc(-c3ccc4c5ccccc5c5c(ccc6oc7ccccc7c65)c4c3)c2)cc1 ; c1ccc(N(c2cccc(-c3ccc4c5ccccc5c5c(ccc6oc7ccccc7c65)c4c3)c2)c2ccc3ccccc3c2)cc1 ; c1ccc(N(c2cccc(-c3ccc4c5ccccc5c5c(ccc6oc7ccccc7c65)c4c3)c2)c2cccc3ccccc23)cc1]

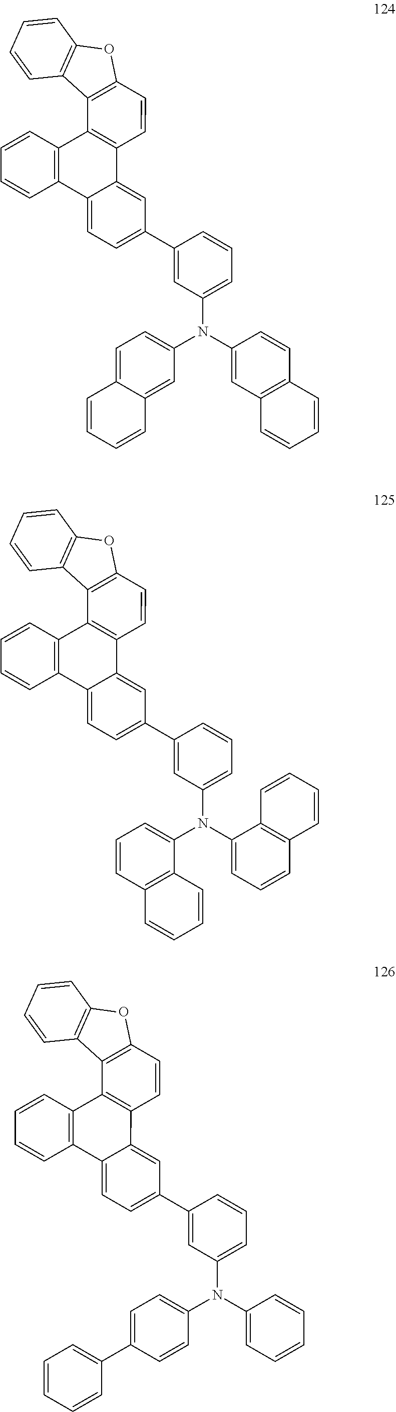 [SMILES: c1cc(-c2ccc3c4ccccc4c4c(ccc5oc6ccccc6c54)c3c2)cc(N(c2ccc3ccccc3c2)c2ccc3ccccc3c2)c1 ; c1cc(-c2ccc3c4ccccc4c4c(ccc5oc6ccccc6c54)c3c2)cc(N(c2cccc3ccccc23)c2cccc3ccccc23)c1 ; c1ccc(-c2ccc(N(c3ccccc3)c3cccc(-c4ccc5c6ccccc6c6c(ccc7oc8ccccc8c76)c5c4)c3)cc2)cc1]

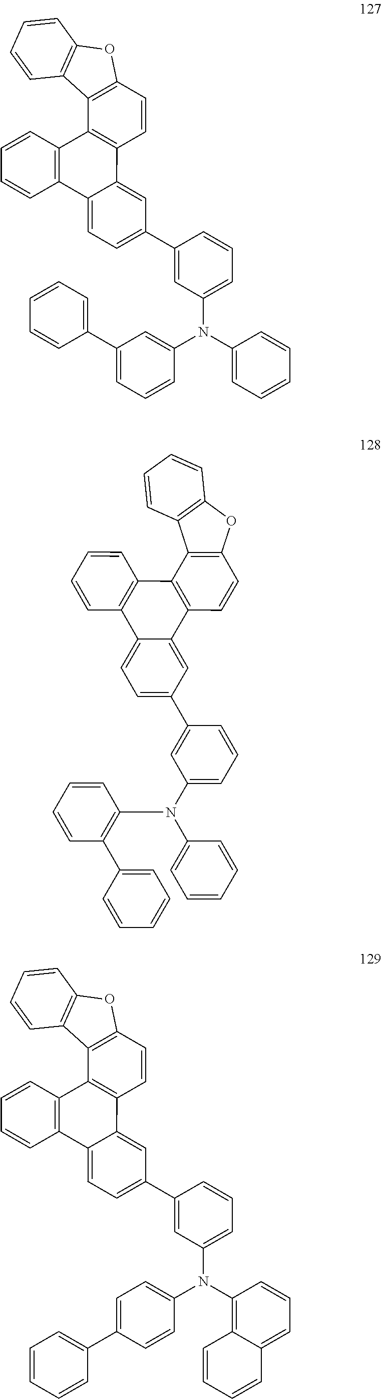 [SMILES: c1ccc(-c2cccc(N(c3ccccc3)c3cccc(-c4ccc5c6ccccc6c6c(ccc7oc8ccccc8c76)c5c4)c3)c2)cc1 ; c1ccc(-c2ccccc2N(c2ccccc2)c2cccc(-c3ccc4c5ccccc5c5c(ccc6oc7ccccc7c65)c4c3)c2)cc1 ; c1ccc(-c2ccc(N(c3cccc(-c4ccc5c6ccccc6c6c(ccc7oc8ccccc8c76)c5c4)c3)c3cccc4ccccc34)cc2)cc1]

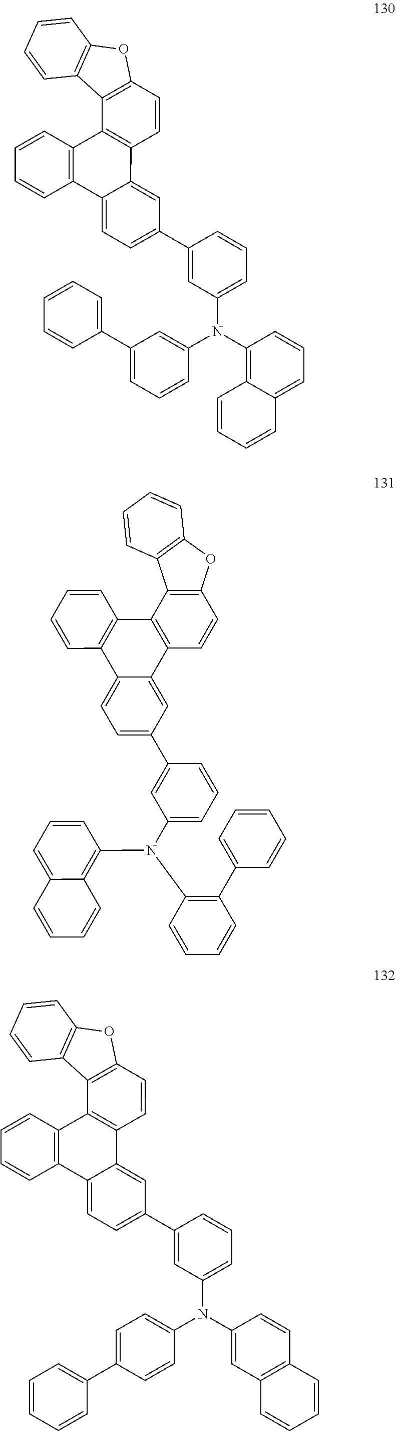 [SMILES: c1ccc(-c2cccc(N(c3cccc(-c4ccc5c6ccccc6c6c(ccc7oc8ccccc8c76)c5c4)c3)c3cccc4ccccc34)c2)cc1 ; c1ccc(-c2ccccc2N(c2cccc(-c3ccc4c5ccccc5c5c(ccc6oc7ccccc7c65)c4c3)c2)c2cccc3ccccc23)cc1 ; c1ccc(-c2ccc(N(c3cccc(-c4ccc5c6ccccc6c6c(ccc7oc8ccccc8c76)c5c4)c3)c3ccc4ccccc4c3)cc2)cc1]

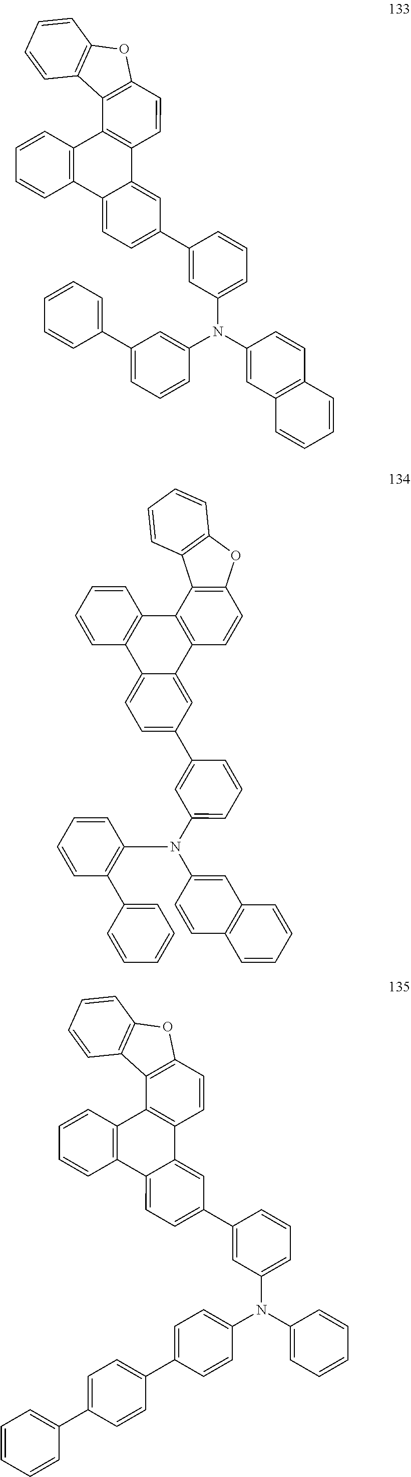

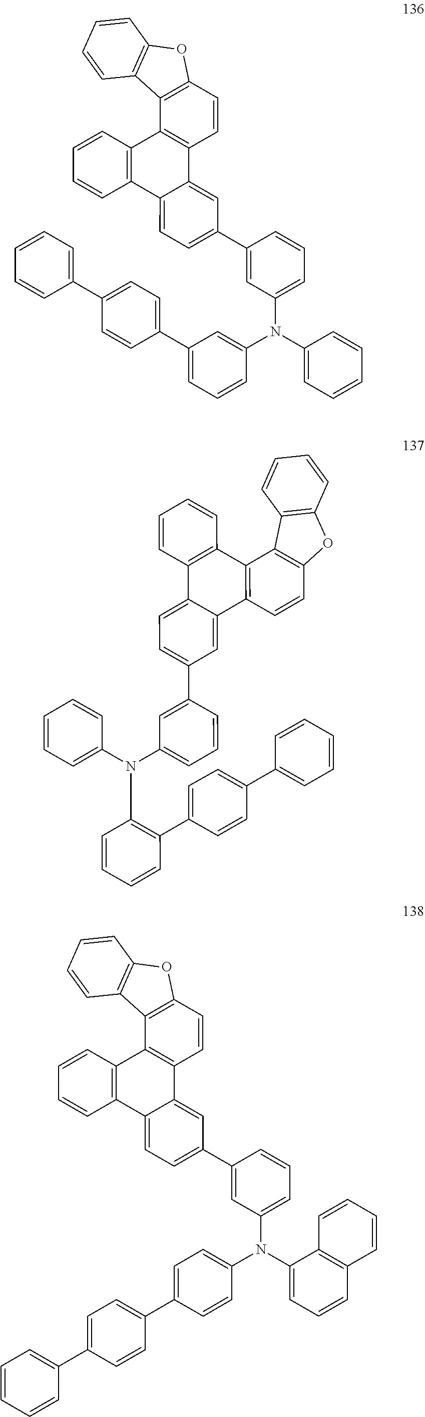 [SMILES: c1ccc(-c2ccc(-c3cccc(N(c4ccccc4)c4cccc(-c5ccc6c7ccccc7c7c(ccc8oc9ccccc9c87)c6c5)c4)c3)cc2)cc1 ; c1ccc(-c2ccc(-c3ccccc3N(c3ccccc3)c3cccc(-c4ccc5c6ccccc6c6c(ccc7oc8ccccc8c76)c5c4)c3)cc2)cc1 ; c1ccc(-c2ccc(-c3ccc(N(c4cccc(-c5ccc6c7ccccc7c7c(ccc8oc9ccccc9c87)c6c5)c4)c4cccc5ccccc45)cc3)cc2)cc1]

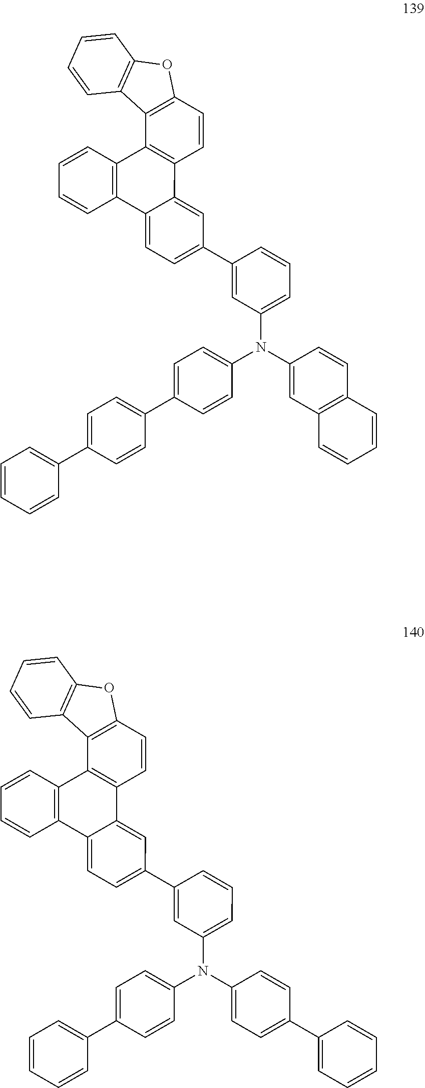 [SMILES: c1ccc(-c2ccc(-c3ccc(N(c4cccc(-c5ccc6c7ccccc7c7c(ccc8oc9ccccc9c87)c6c5)c4)c4ccc5ccccc5c4)cc3)cc2)cc1 ; c1ccc(-c2ccc(N(c3ccc(-c4ccccc4)cc3)c3cccc(-c4ccc5c6ccccc6c6c(ccc7oc8ccccc8c76)c5c4)c3)cc2)cc1]

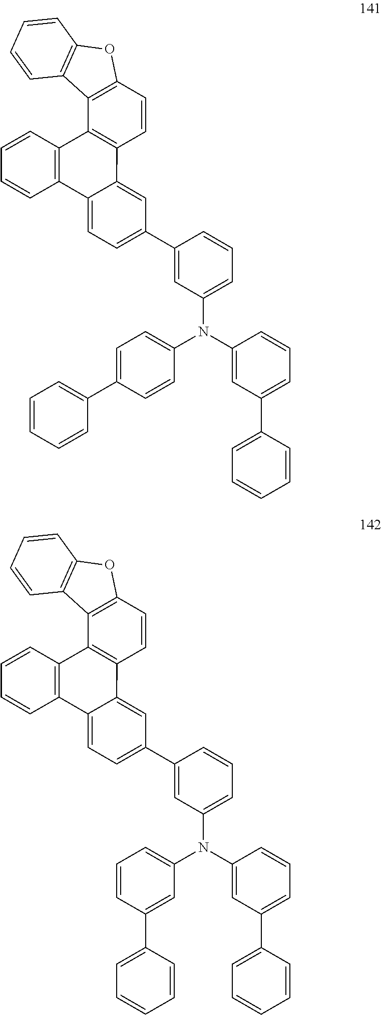 [SMILES: c1ccc(-c2ccc(N(c3cccc(-c4ccccc4)c3)c3cccc(-c4ccc5c6ccccc6c6c(ccc7oc8ccccc8c76)c5c4)c3)cc2)cc1 ; c1ccc(-c2cccc(N(c3cccc(-c4ccccc4)c3)c3cccc(-c4ccc5c6ccccc6c6c(ccc7oc8ccccc8c76)c5c4)c3)c2)cc1]

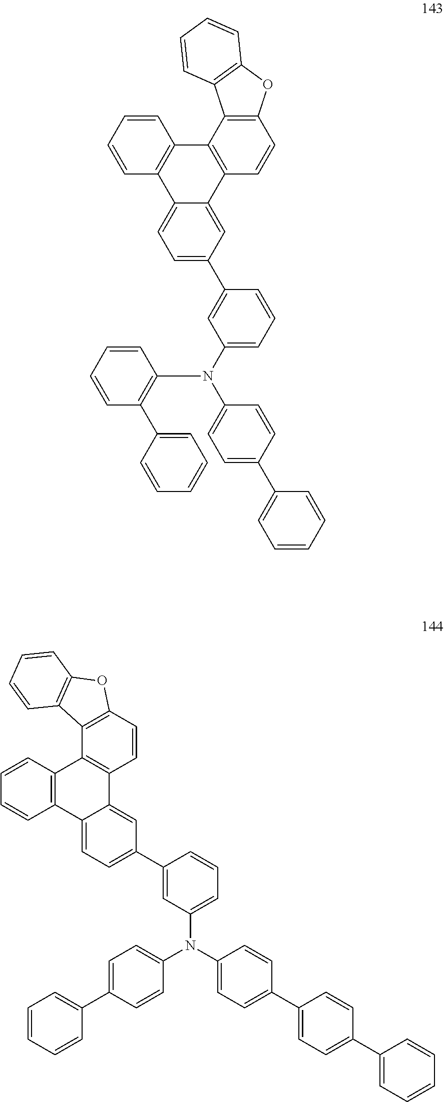 [SMILES: c1ccc(-c2ccc(N(c3cccc(-c4ccc5c6ccccc6c6c(ccc7oc8ccccc8c76)c5c4)c3)c3ccccc3-c3ccccc3)cc2)cc1 ; c1ccc(-c2ccc(-c3ccc(N(c4ccc(-c5ccccc5)cc4)c4cccc(-c5ccc6c7ccccc7c7c(ccc8oc9ccccc9c87)c6c5)c4)cc3)cc2)cc1]

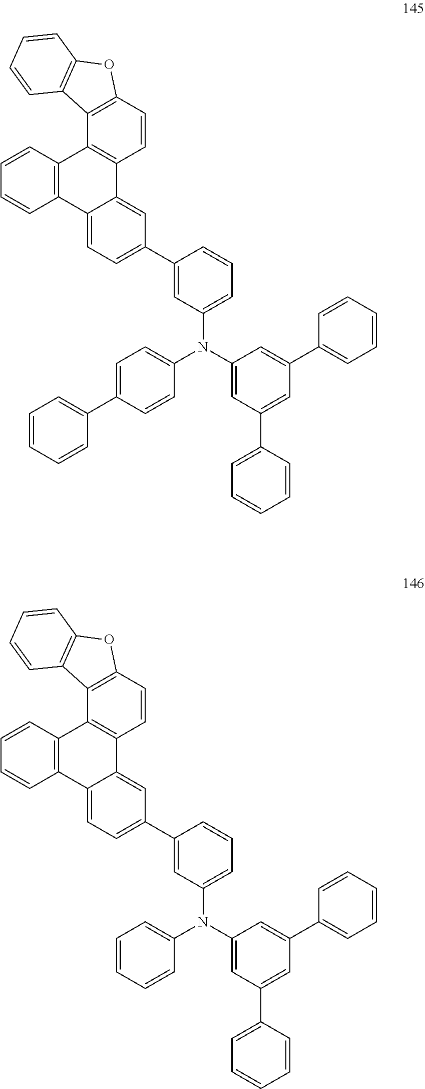 [SMILES: c1ccc(-c2ccc(N(c3cccc(-c4ccc5c6ccccc6c6c(ccc7oc8ccccc8c76)c5c4)c3)c3cc(-c4ccccc4)cc(-c4ccccc4)c3)cc2)cc1 ; c1ccc(-c2cc(-c3ccccc3)cc(N(c3ccccc3)c3cccc(-c4ccc5c6ccccc6c6c(ccc7oc8ccccc8c76)c5c4)c3)c2)cc1]

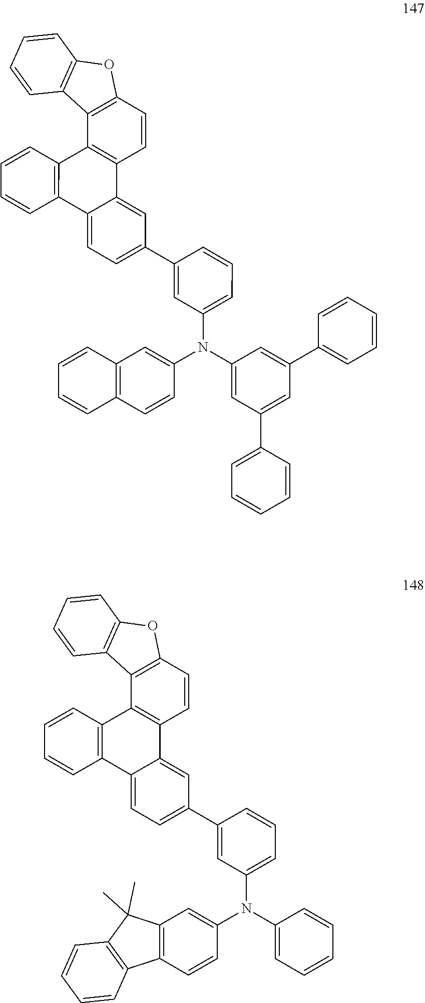 [SMILES: c1ccc(-c2cc(-c3ccccc3)cc(N(c3cccc(-c4ccc5c6ccccc6c6c(ccc7oc8ccccc8c76)c5c4)c3)c3ccc4ccccc4c3)c2)cc1 ; CC1(C)c2ccccc2-c2ccc(N(c3ccccc3)c3cccc(-c4ccc5c6ccccc6c6c(ccc7oc8ccccc8c76)c5c4)c3)cc21]

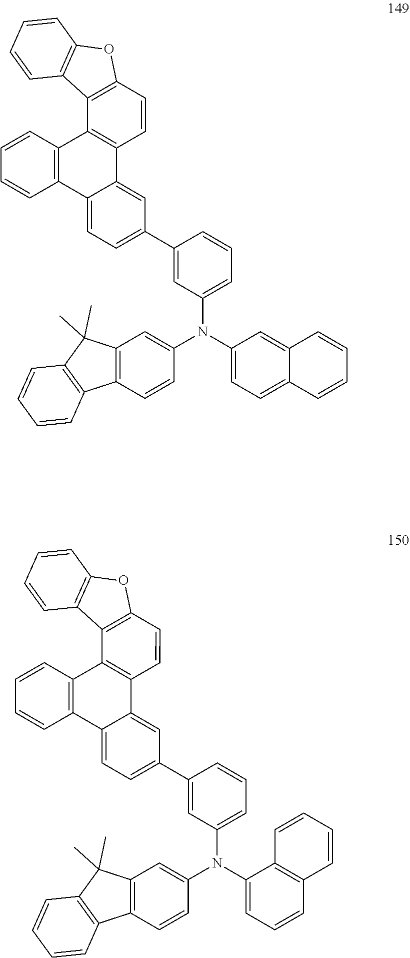 [SMILES: CC1(C)c2ccccc2-c2ccc(N(c3cccc(-c4ccc5c6ccccc6c6c(ccc7oc8ccccc8c76)c5c4)c3)c3ccc4ccccc4c3)cc21 ; CC1(C)c2ccccc2-c2ccc(N(c3cccc(-c4ccc5c6ccccc6c6c(ccc7oc8ccccc8c76)c5c4)c3)c3cccc4ccccc34)cc21]

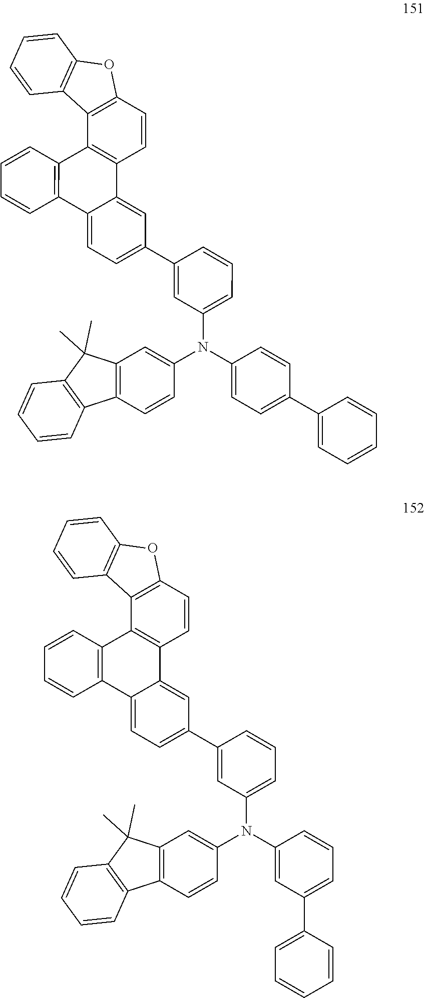 [SMILES: CC1(C)c2ccccc2-c2ccc(N(c3ccc(-c4ccccc4)cc3)c3cccc(-c4ccc5c6ccccc6c6c(ccc7oc8ccccc8c76)c5c4)c3)cc21 ; CC1(C)c2ccccc2-c2ccc(N(c3cccc(-c4ccccc4)c3)c3cccc(-c4ccc5c6ccccc6c6c(ccc7oc8ccccc8c76)c5c4)c3)cc21]

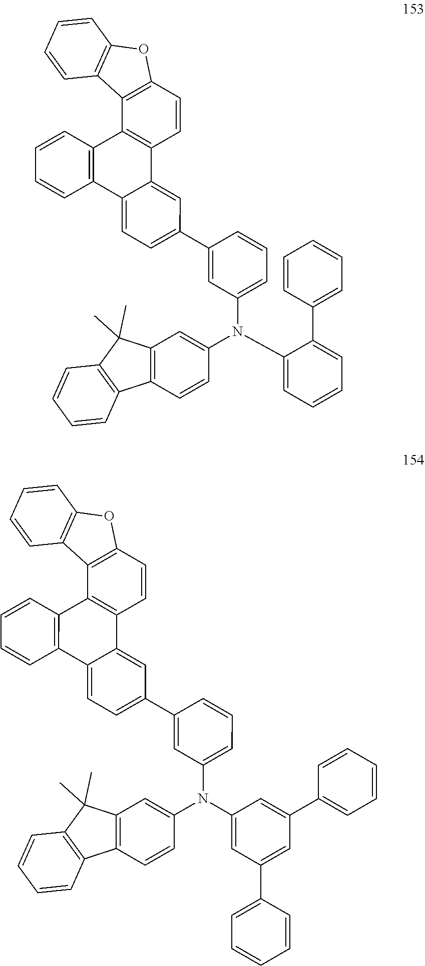 [SMILES: CC1(C)c2ccccc2-c2ccc(N(c3cccc(-c4ccc5c6ccccc6c6c(ccc7oc8ccccc8c76)c5c4)c3)c3ccccc3-c3ccccc3)cc21 ; CC1(C)c2ccccc2-c2ccc(N(c3cccc(-c4ccc5c6ccccc6c6c(ccc7oc8ccccc8c76)c5c4)c3)c3cc(-c4ccccc4)cc(-c4ccccc4)c3)cc21]

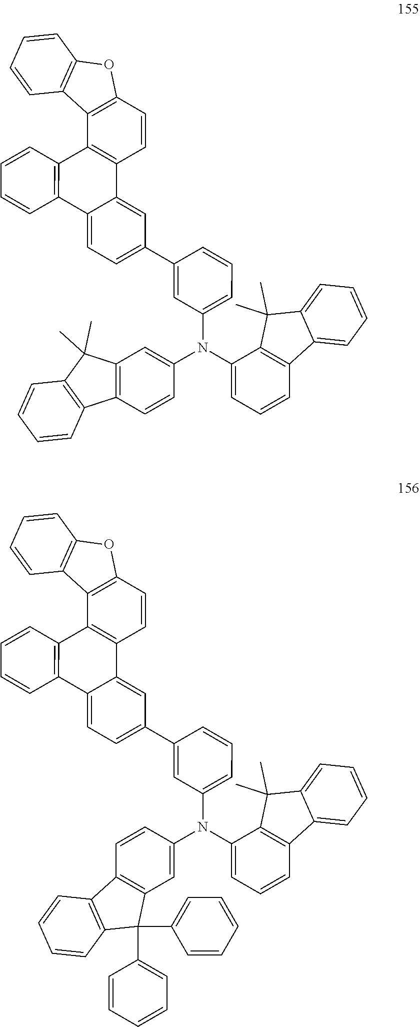 [SMILES: CC1(C)c2ccccc2-c2ccc(N(c3cccc(-c4ccc5c6ccccc6c6c(ccc7oc8ccccc8c76)c5c4)c3)c3cccc4c3C(C)(C)c3ccccc3-4)cc21 ; CC1(C)c2ccccc2-c2cccc(N(c3cccc(-c4ccc5c6ccccc6c6c(ccc7oc8ccccc8c76)c5c4)c3)c3ccc4c(c3)C(c3ccccc3)(c3ccccc3)c3ccccc3-4)c21]

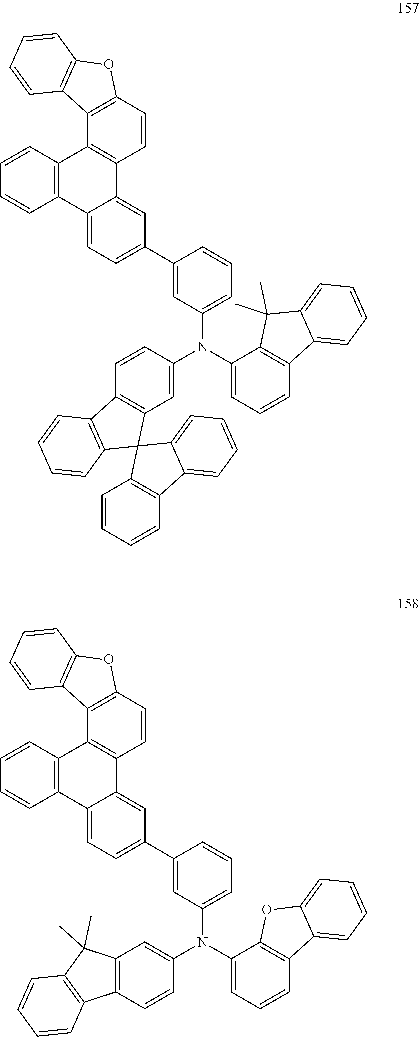 [SMILES: CC1(C)c2ccccc2-c2cccc(N(c3cccc(-c4ccc5c6ccccc6c6c(ccc7oc8ccccc8c76)c5c4)c3)c3ccc4c(c3)C3(c5ccccc5-c5ccccc53)c3ccccc3-4)c21 ; CC1(C)c2ccccc2-c2ccc(N(c3cccc(-c4ccc5c6ccccc6c6c(ccc7oc8ccccc8c76)c5c4)c3)c3cccc4c3oc3ccccc34)cc21]

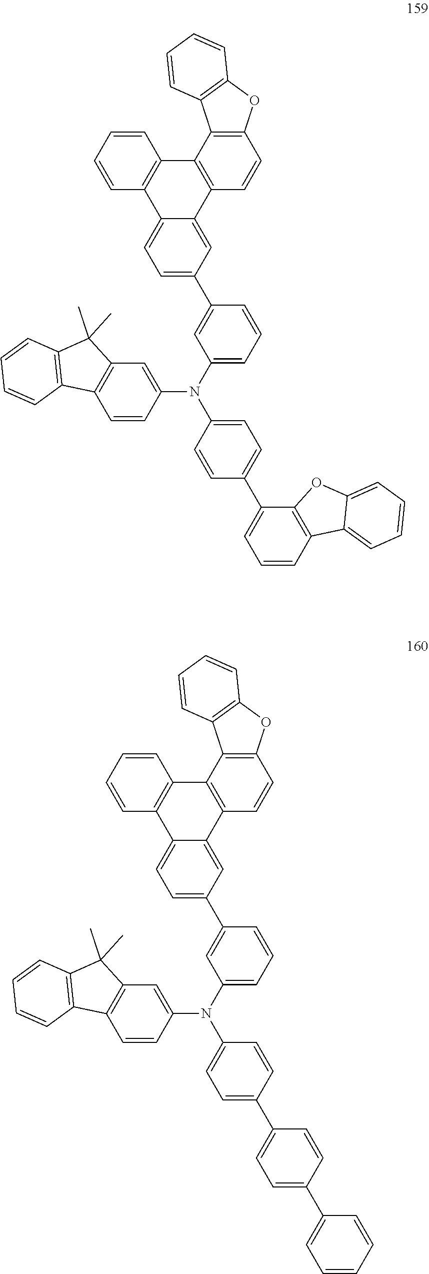 [SMILES: CC1(C)c2ccccc2-c2ccc(N(c3ccc(-c4cccc5c4oc4ccccc45)cc3)c3cccc(-c4ccc5c6ccccc6c6c(ccc7oc8ccccc8c76)c5c4)c3)cc21 ; CC1(C)c2ccccc2-c2ccc(N(c3ccc(-c4ccc(-c5ccccc5)cc4)cc3)c3cccc(-c4ccc5c6ccccc6c6c(ccc7oc8ccccc8c76)c5c4)c3)cc21]

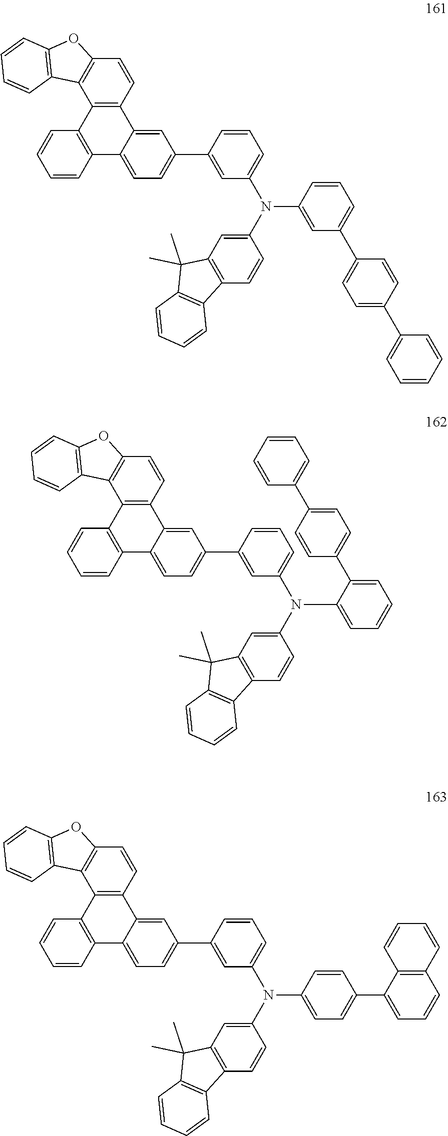 [SMILES: CC1(C)c2ccccc2-c2ccc(N(c3cccc(-c4ccc(-c5ccccc5)cc4)c3)c3cccc(-c4ccc5c6ccccc6c6c(ccc7oc8ccccc8c76)c5c4)c3)cc21 ; CC1(C)c2ccccc2-c2ccc(N(c3cccc(-c4ccc5c6ccccc6c6c(ccc7oc8ccccc8c76)c5c4)c3)c3ccccc3-c3ccc(-c4ccccc4)cc3)cc21 ; CC1(C)c2ccccc2-c2ccc(N(c3ccc(-c4cccc5ccccc45)cc3)c3cccc(-c4ccc5c6ccccc6c6c(ccc7oc8ccccc8c76)c5c4)c3)cc21]

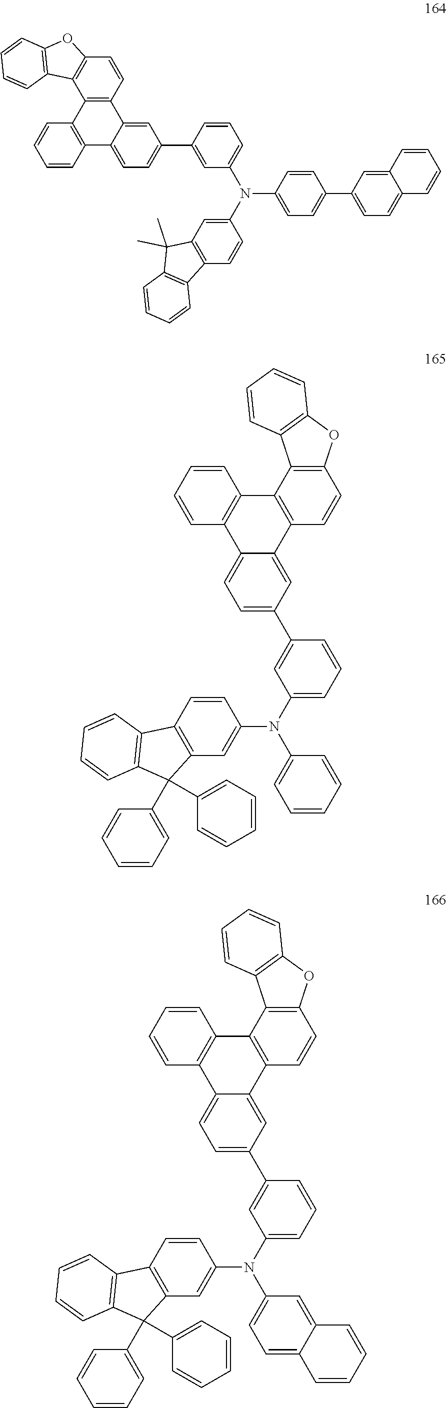 [SMILES: CC1(C)c2ccccc2-c2ccc(N(c3ccc(-c4ccc5ccccc5c4)cc3)c3cccc(-c4ccc5c6ccccc6c6c(ccc7oc8ccccc8c76)c5c4)c3)cc21 ; c1ccc(N(c2cccc(-c3ccc4c5ccccc5c5c(ccc6oc7ccccc7c65)c4c3)c2)c2ccc3c(c2)C(c2ccccc2)(c2ccccc2)c2ccccc2-3)cc1 ; c1ccc(C2(c3ccccc3)c3ccccc3-c3ccc(N(c4cccc(-c5ccc6c7ccccc7c7c(ccc8oc9ccccc9c87)c6c5)c4)c4ccc5ccccc5c4)cc32)cc1]

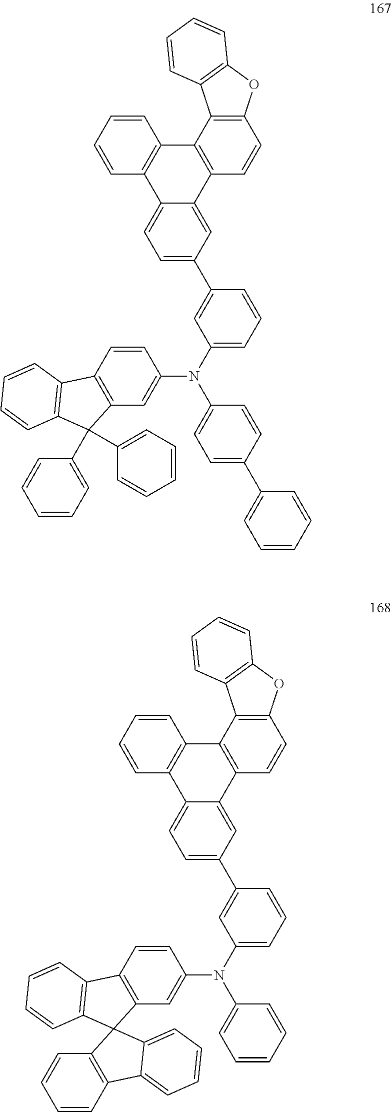 [SMILES: c1ccc(-c2ccc(N(c3cccc(-c4ccc5c6ccccc6c6c(ccc7oc8ccccc8c76)c5c4)c3)c3ccc4c(c3)C(c3ccccc3)(c3ccccc3)c3ccccc3-4)cc2)cc1 ; c1ccc(N(c2cccc(-c3ccc4c5ccccc5c5c(ccc6oc7ccccc7c65)c4c3)c2)c2ccc3c(c2)C2(c4ccccc4-c4ccccc42)c2ccccc2-3)cc1]

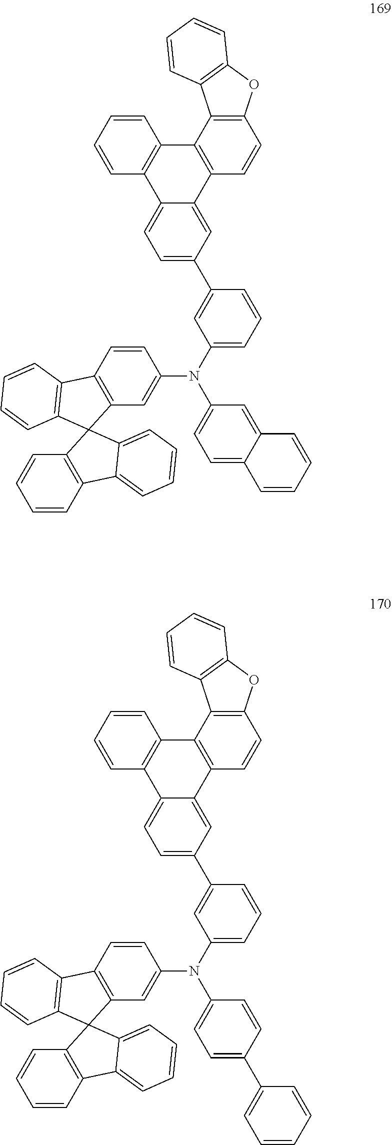 [SMILES: c1cc(-c2ccc3c4ccccc4c4c(ccc5oc6ccccc6c54)c3c2)cc(N(c2ccc3c(c2)C2(c4ccccc4-c4ccccc42)c2ccccc2-3)c2ccc3ccccc3c2)c1 ; c1ccc(-c2ccc(N(c3cccc(-c4ccc5c6ccccc6c6c(ccc7oc8ccccc8c76)c5c4)c3)c3ccc4c(c3)C3(c5ccccc5-c5ccccc53)c3ccccc3-4)cc2)cc1]

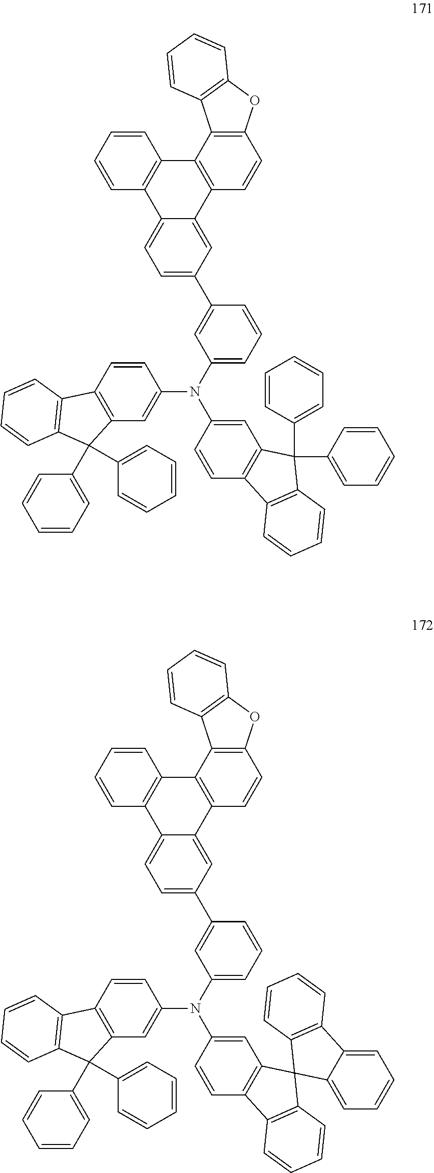 [SMILES: c1ccc(C2(c3ccccc3)c3ccccc3-c3ccc(N(c4cccc(-c5ccc6c7ccccc7c7c(ccc8oc9ccccc9c87)c6c5)c4)c4ccc5c(c4)C(c4ccccc4)(c4ccccc4)c4ccccc4-5)cc32)cc1 ; c1ccc(C2(c3ccccc3)c3ccccc3-c3ccc(N(c4cccc(-c5ccc6c7ccccc7c7c(ccc8oc9ccccc9c87)c6c5)c4)c4ccc5c(c4)C4(c6ccccc6-c6ccccc64)c4ccccc4-5)cc32)cc1]

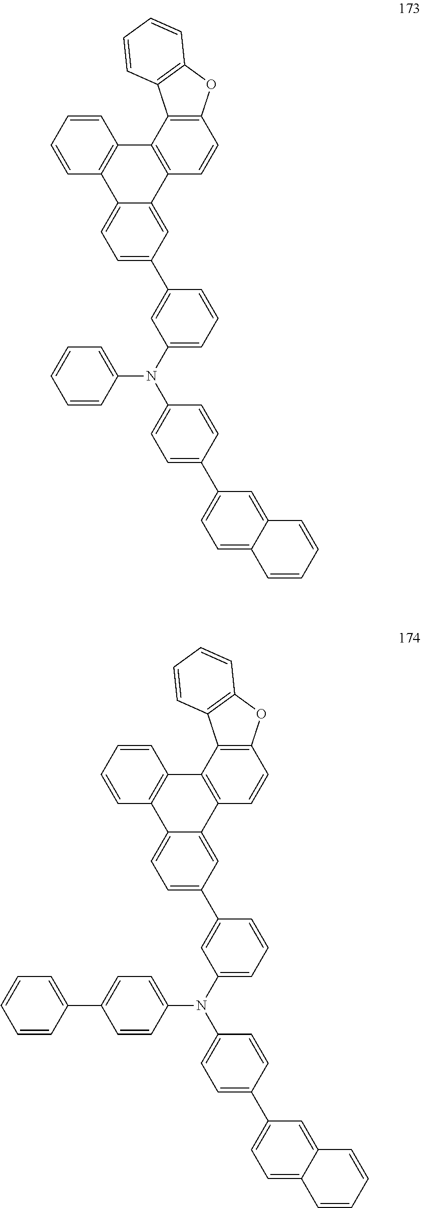 [SMILES: c1ccc(N(c2ccc(-c3ccc4ccccc4c3)cc2)c2cccc(-c3ccc4c5ccccc5c5c(ccc6oc7ccccc7c65)c4c3)c2)cc1 ; c1ccc(-c2ccc(N(c3ccc(-c4ccc5ccccc5c4)cc3)c3cccc(-c4ccc5c6ccccc6c6c(ccc7oc8ccccc8c76)c5c4)c3)cc2)cc1]

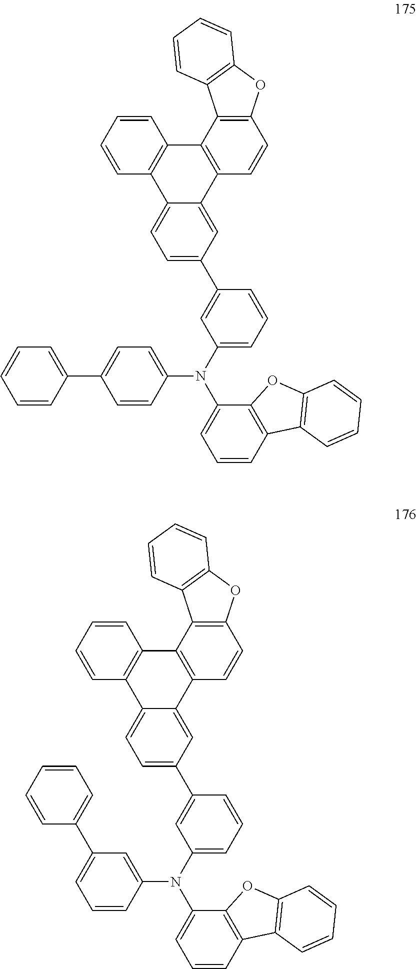 [SMILES: c1ccc(-c2ccc(N(c3cccc(-c4ccc5c6ccccc6c6c(ccc7oc8ccccc8c76)c5c4)c3)c3cccc4c3oc3ccccc34)cc2)cc1 ; c1ccc(-c2cccc(N(c3cccc(-c4ccc5c6ccccc6c6c(ccc7oc8ccccc8c76)c5c4)c3)c3cccc4c3oc3ccccc34)c2)cc1]

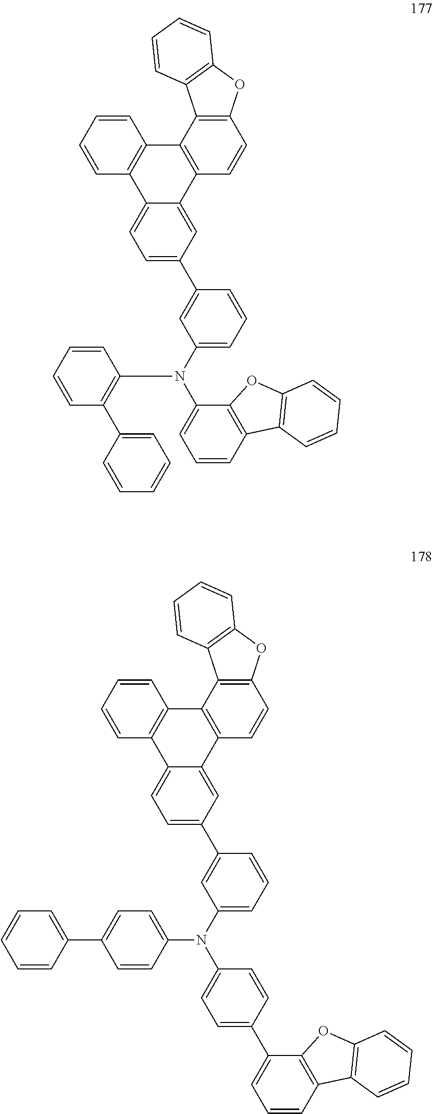 [SMILES: c1ccc(-c2ccccc2N(c2cccc(-c3ccc4c5ccccc5c5c(ccc6oc7ccccc7c65)c4c3)c2)c2cccc3c2oc2ccccc23)cc1 ; c1ccc(-c2ccc(N(c3ccc(-c4cccc5c4oc4ccccc45)cc3)c3cccc(-c4ccc5c6ccccc6c6c(ccc7oc8ccccc8c76)c5c4)c3)cc2)cc1]

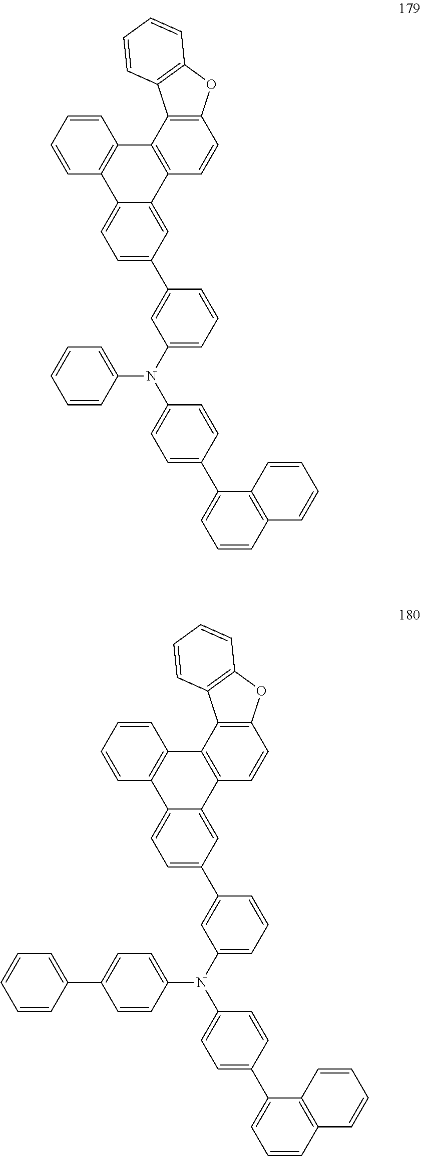 [SMILES: c1ccc(N(c2ccc(-c3cccc4ccccc34)cc2)c2cccc(-c3ccc4c5ccccc5c5c(ccc6oc7ccccc7c65)c4c3)c2)cc1 ; c1ccc(-c2ccc(N(c3ccc(-c4cccc5ccccc45)cc3)c3cccc(-c4ccc5c6ccccc6c6c(ccc7oc8ccccc8c76)c5c4)c3)cc2)cc1]

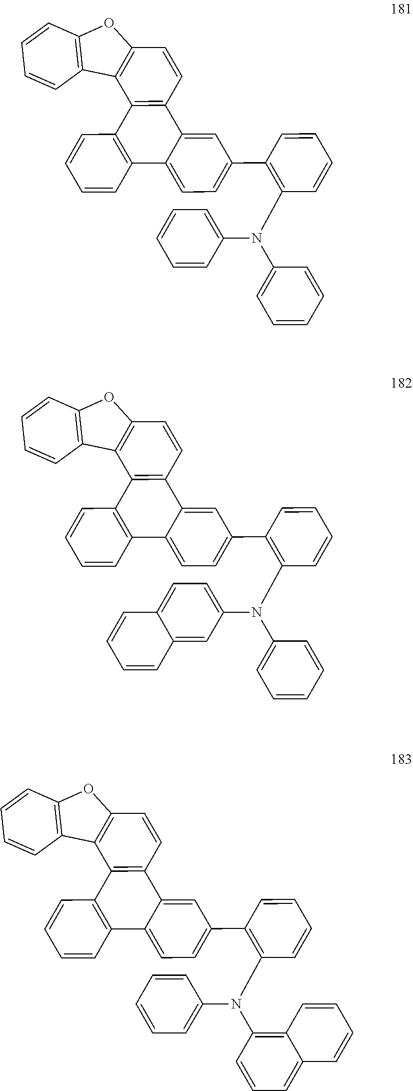 [SMILES: c1ccc(N(c2ccccc2)c2ccccc2-c2ccc3c4ccccc4c4c(ccc5oc6ccccc6c54)c3c2)cc1 ; c1ccc(N(c2ccc3ccccc3c2)c2ccccc2-c2ccc3c4ccccc4c4c(ccc5oc6ccccc6c54)c3c2)cc1 ; c1ccc(N(c2ccccc2-c2ccc3c4ccccc4c4c(ccc5oc6ccccc6c54)c3c2)c2cccc3ccccc23)cc1]

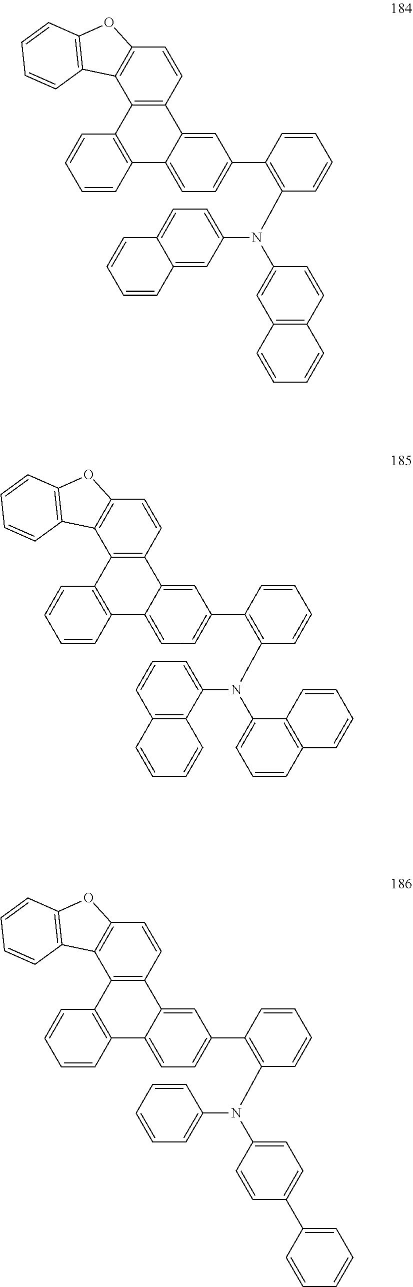 [SMILES: c1ccc(N(c2ccc3ccccc3c2)c2ccc3ccccc3c2)c(-c2ccc3c4ccccc4c4c(ccc5oc6ccccc6c54)c3c2)c1 ; c1ccc(N(c2cccc3ccccc23)c2cccc3ccccc23)c(-c2ccc3c4ccccc4c4c(ccc5oc6ccccc6c54)c3c2)c1 ; c1ccc(-c2ccc(N(c3ccccc3)c3ccccc3-c3ccc4c5ccccc5c5c(ccc6oc7ccccc7c65)c4c3)cc2)cc1]

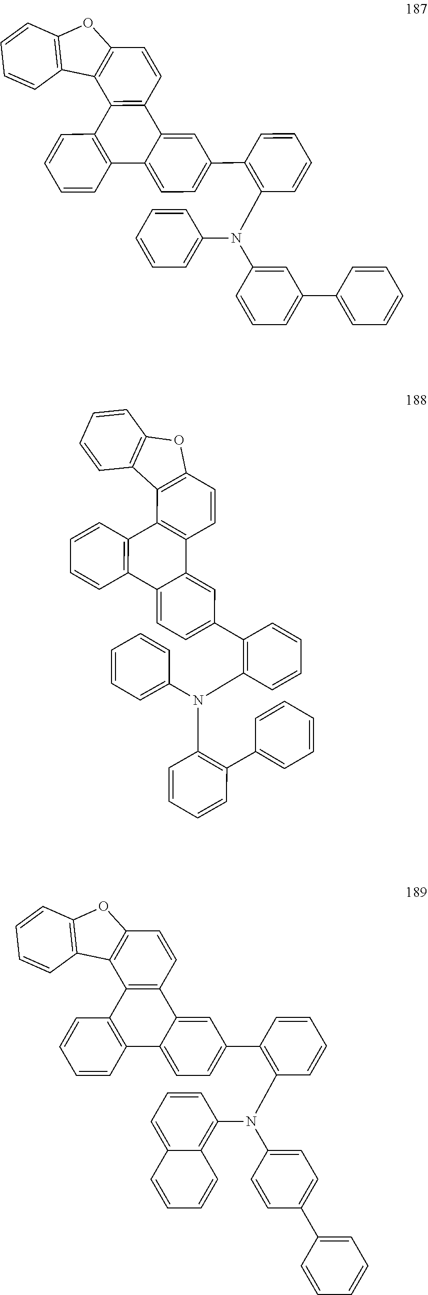 [SMILES: c1ccc(-c2cccc(N(c3ccccc3)c3ccccc3-c3ccc4c5ccccc5c5c(ccc6oc7ccccc7c65)c4c3)c2)cc1 ; c1ccc(-c2ccccc2N(c2ccccc2)c2ccccc2-c2ccc3c4ccccc4c4c(ccc5oc6ccccc6c54)c3c2)cc1 ; c1ccc(-c2ccc(N(c3ccccc3-c3ccc4c5ccccc5c5c(ccc6oc7ccccc7c65)c4c3)c3cccc4ccccc34)cc2)cc1]

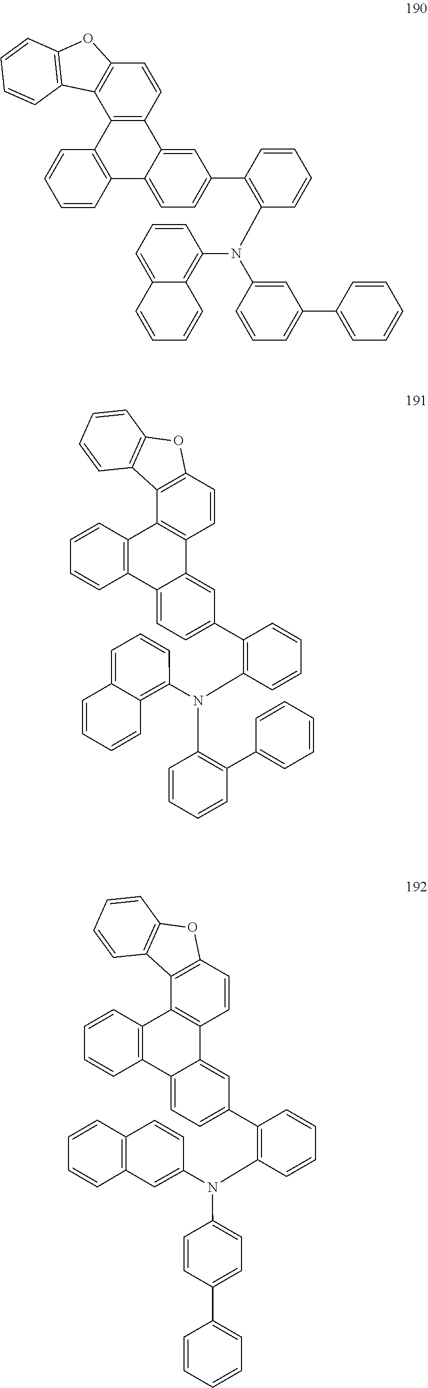 [SMILES: c1ccc(-c2cccc(N(c3ccccc3-c3ccc4c5ccccc5c5c(ccc6oc7ccccc7c65)c4c3)c3cccc4ccccc34)c2)cc1 ; c1ccc(-c2ccccc2N(c2ccccc2-c2ccc3c4ccccc4c4c(ccc5oc6ccccc6c54)c3c2)c2cccc3ccccc23)cc1 ; c1ccc(-c2ccc(N(c3ccc4ccccc4c3)c3ccccc3-c3ccc4c5ccccc5c5c(ccc6oc7ccccc7c65)c4c3)cc2)cc1]

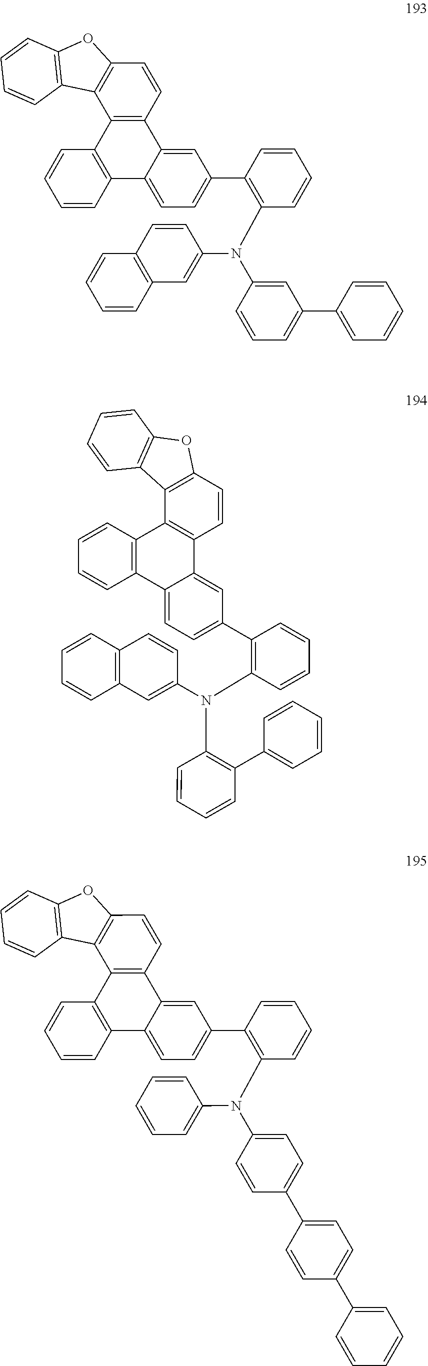 [SMILES: c1ccc(-c2cccc(N(c3ccc4ccccc4c3)c3ccccc3-c3ccc4c5ccccc5c5c(ccc6oc7ccccc7c65)c4c3)c2)cc1 ; c1ccc(-c2ccccc2N(c2ccc3ccccc3c2)c2ccccc2-c2ccc3c4ccccc4c4c(ccc5oc6ccccc6c54)c3c2)cc1 ; c1ccc(-c2ccc(-c3ccc(N(c4ccccc4)c4ccccc4-c4ccc5c6ccccc6c6c(ccc7oc8ccccc8c76)c5c4)cc3)cc2)cc1]

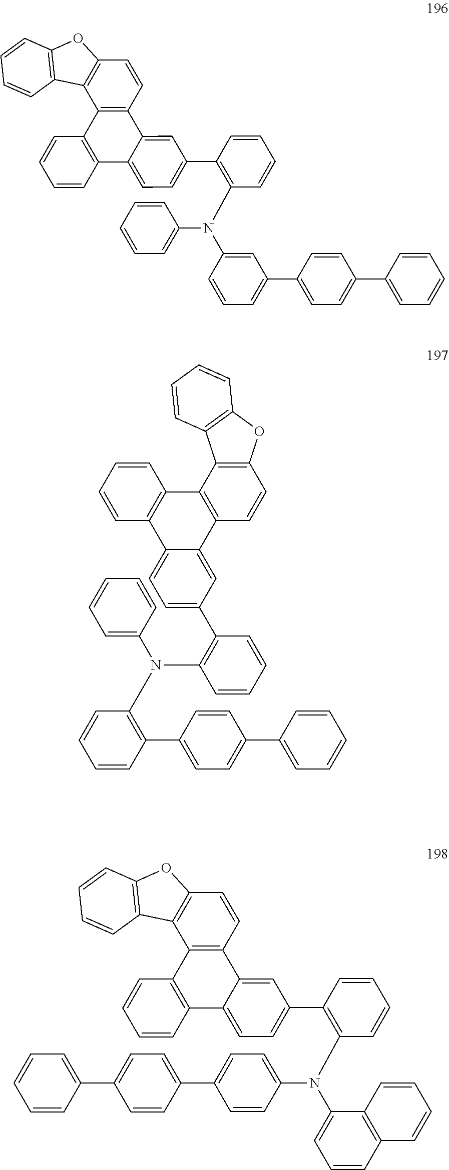 [SMILES: c1ccc(-c2ccc(-c3cccc(N(c4ccccc4)c4ccccc4-c4ccc5c6ccccc6c6c(ccc7oc8ccccc8c76)c5c4)c3)cc2)cc1 ; c1ccc(-c2ccc(-c3ccccc3N(c3ccccc3)c3ccccc3-c3ccc4c5ccccc5c5c(ccc6oc7ccccc7c65)c4c3)cc2)cc1 ; c1ccc(-c2ccc(-c3ccc(N(c4ccccc4-c4ccc5c6ccccc6c6c(ccc7oc8ccccc8c76)c5c4)c4cccc5ccccc45)cc3)cc2)cc1]

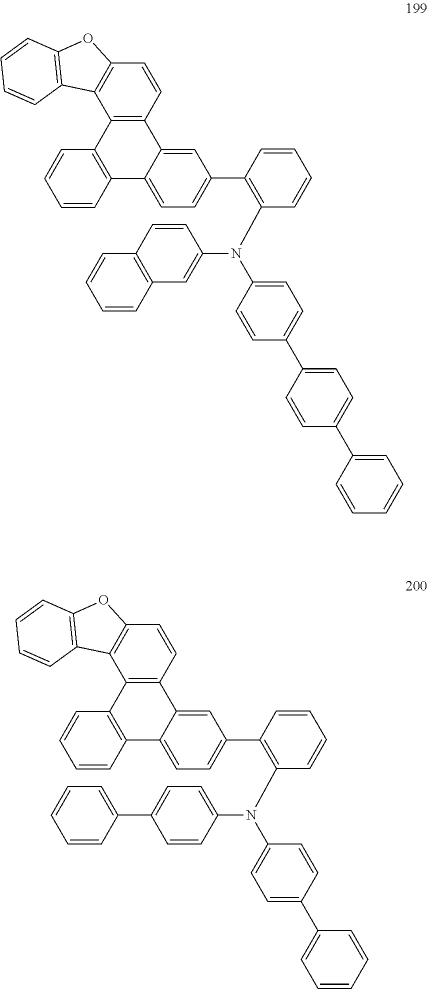 [SMILES: c1ccc(-c2ccc(-c3ccc(N(c4ccc5ccccc5c4)c4ccccc4-c4ccc5c6ccccc6c6c(ccc7oc8ccccc8c76)c5c4)cc3)cc2)cc1 ; c1ccc(-c2ccc(N(c3ccc(-c4ccccc4)cc3)c3ccccc3-c3ccc4c5ccccc5c5c(ccc6oc7ccccc7c65)c4c3)cc2)cc1]

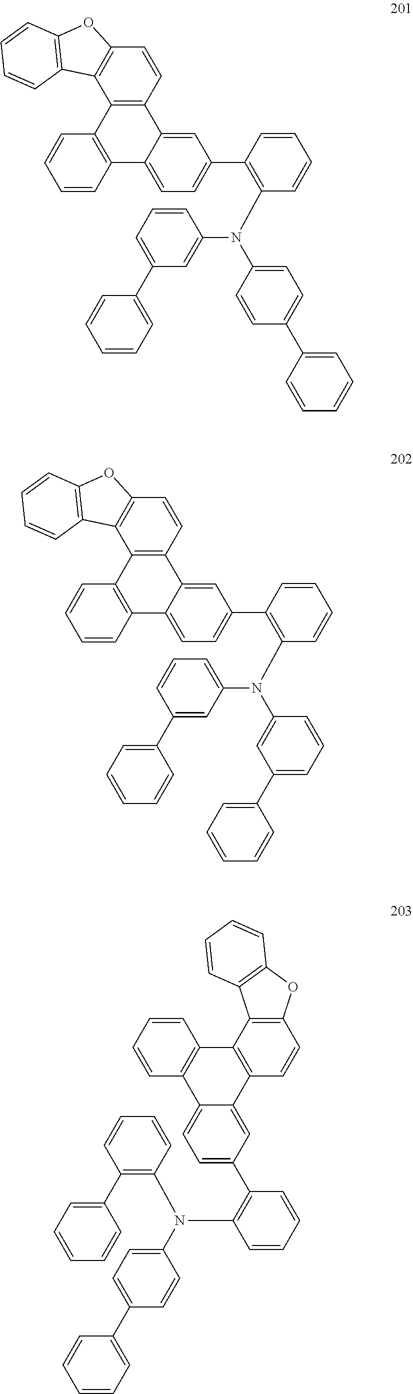 [SMILES: c1ccc(-c2ccc(N(c3cccc(-c4ccccc4)c3)c3ccccc3-c3ccc4c5ccccc5c5c(ccc6oc7ccccc7c65)c4c3)cc2)cc1 ; c1ccc(-c2cccc(N(c3cccc(-c4ccccc4)c3)c3ccccc3-c3ccc4c5ccccc5c5c(ccc6oc7ccccc7c65)c4c3)c2)cc1 ; c1ccc(-c2ccc(N(c3ccccc3-c3ccccc3)c3ccccc3-c3ccc4c5ccccc5c5c(ccc6oc7ccccc7c65)c4c3)cc2)cc1]

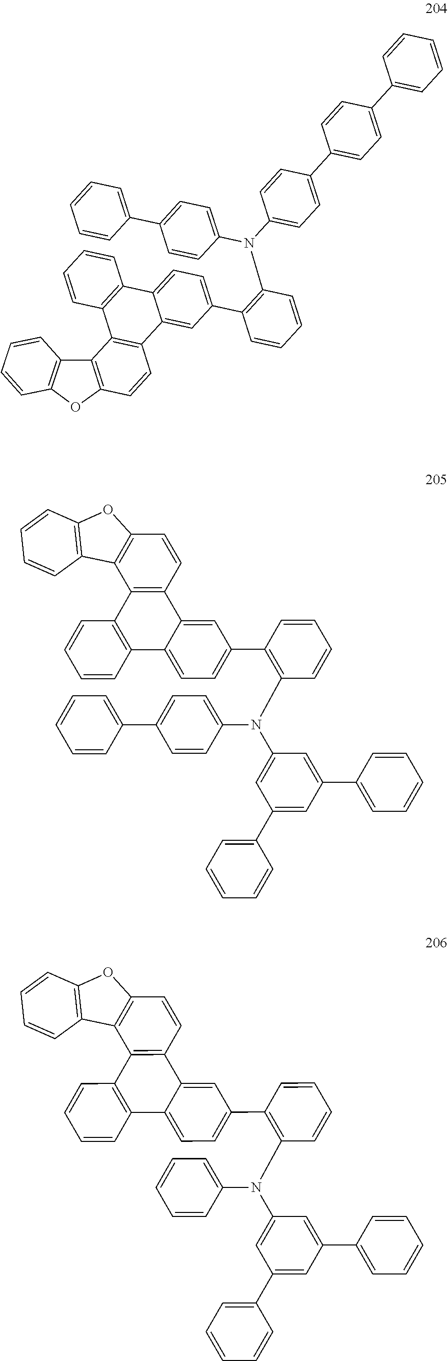 [SMILES: c1ccc(-c2ccc(-c3ccc(N(c4ccc(-c5ccccc5)cc4)c4ccccc4-c4ccc5c6ccccc6c6c(ccc7oc8ccccc8c76)c5c4)cc3)cc2)cc1 ; c1ccc(-c2ccc(N(c3cc(-c4ccccc4)cc(-c4ccccc4)c3)c3ccccc3-c3ccc4c5ccccc5c5c(ccc6oc7ccccc7c65)c4c3)cc2)cc1 ; c1ccc(-c2cc(-c3ccccc3)cc(N(c3ccccc3)c3ccccc3-c3ccc4c5ccccc5c5c(ccc6oc7ccccc7c65)c4c3)c2)cc1]

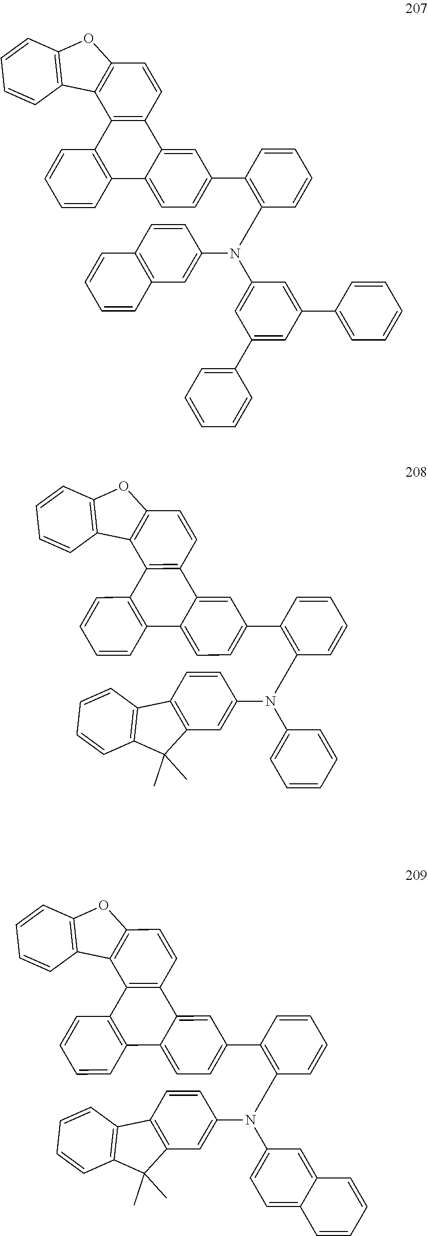 [SMILES: c1ccc(-c2cc(-c3ccccc3)cc(N(c3ccc4ccccc4c3)c3ccccc3-c3ccc4c5ccccc5c5c(ccc6oc7ccccc7c65)c4c3)c2)cc1 ; CC1(C)c2ccccc2-c2ccc(N(c3ccccc3)c3ccccc3-c3ccc4c5ccccc5c5c(ccc6oc7ccccc7c65)c4c3)cc21 ; CC1(C)c2ccccc2-c2ccc(N(c3ccc4ccccc4c3)c3ccccc3-c3ccc4c5ccccc5c5c(ccc6oc7ccccc7c65)c4c3)cc21]

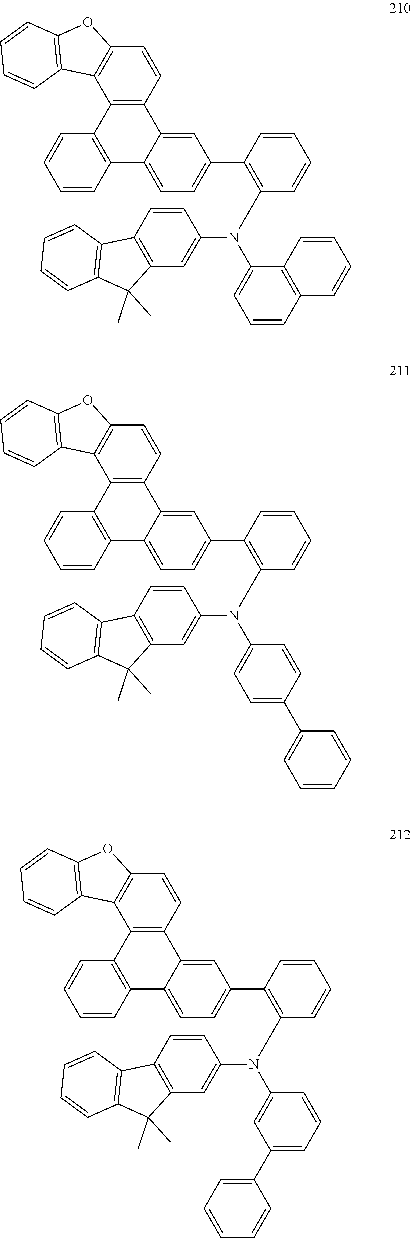 [SMILES: CC1(C)c2ccccc2-c2ccc(N(c3ccccc3-c3ccc4c5ccccc5c5c(ccc6oc7ccccc7c65)c4c3)c3cccc4ccccc34)cc21 ; CC1(C)c2ccccc2-c2ccc(N(c3ccc(-c4ccccc4)cc3)c3ccccc3-c3ccc4c5ccccc5c5c(ccc6oc7ccccc7c65)c4c3)cc21 ; CC1(C)c2ccccc2-c2ccc(N(c3cccc(-c4ccccc4)c3)c3ccccc3-c3ccc4c5ccccc5c5c(ccc6oc7ccccc7c65)c4c3)cc21]

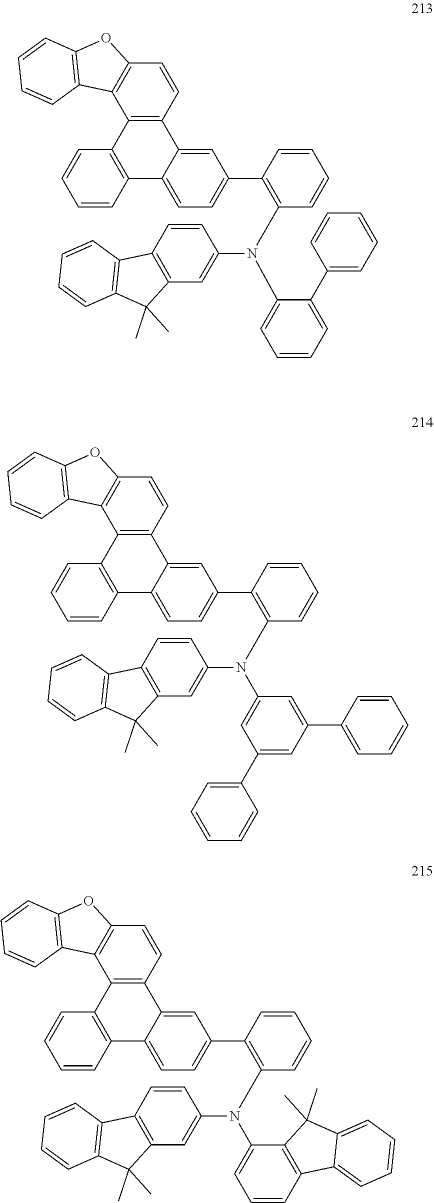 [SMILES: CC1(C)c2ccccc2-c2ccc(N(c3ccccc3-c3ccccc3)c3ccccc3-c3ccc4c5ccccc5c5c(ccc6oc7ccccc7c65)c4c3)cc21 ; CC1(C)c2ccccc2-c2ccc(N(c3cc(-c4ccccc4)cc(-c4ccccc4)c3)c3ccccc3-c3ccc4c5ccccc5c5c(ccc6oc7ccccc7c65)c4c3)cc21 ; CC1(C)c2ccccc2-c2ccc(N(c3ccccc3-c3ccc4c5ccccc5c5c(ccc6oc7ccccc7c65)c4c3)c3cccc4c3C(C)(C)c3ccccc3-4)cc21]

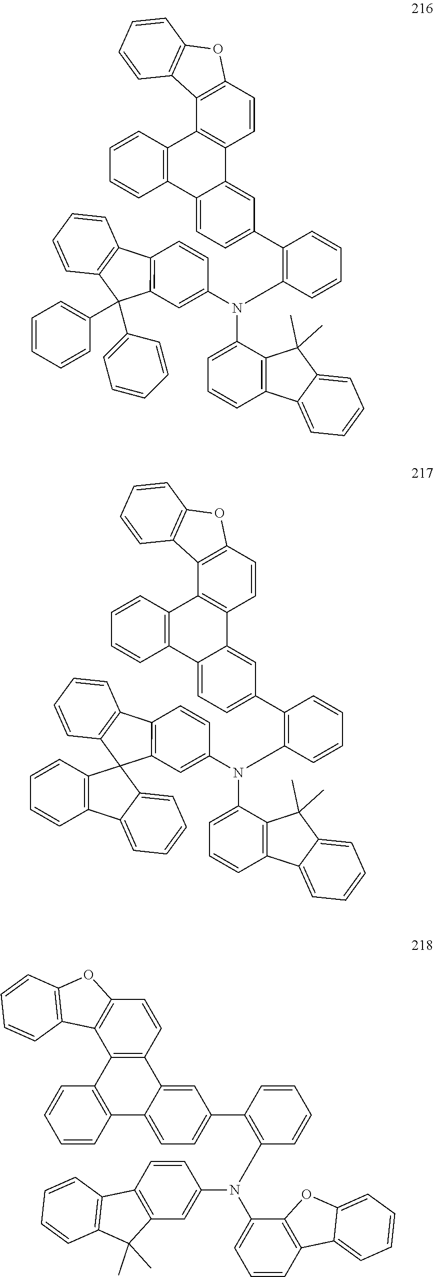 [SMILES: CC1(C)c2ccccc2-c2cccc(N(c3ccc4c(c3)C(c3ccccc3)(c3ccccc3)c3ccccc3-4)c3ccccc3-c3ccc4c5ccccc5c5c(ccc6oc7ccccc7c65)c4c3)c21 ; CC1(C)c2ccccc2-c2cccc(N(c3ccc4c(c3)C3(c5ccccc5-c5ccccc53)c3ccccc3-4)c3ccccc3-c3ccc4c5ccccc5c5c(ccc6oc7ccccc7c65)c4c3)c21 ; CC1(C)c2ccccc2-c2ccc(N(c3ccccc3-c3ccc4c5ccccc5c5c(ccc6oc7ccccc7c65)c4c3)c3cccc4c3oc3ccccc34)cc21]

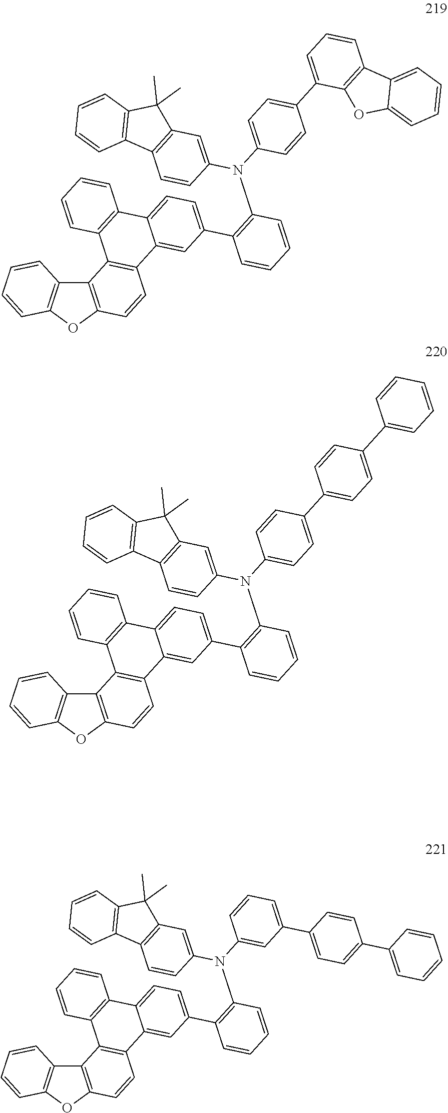 [SMILES: CC1(C)c2ccccc2-c2ccc(N(c3ccc(-c4cccc5c4oc4ccccc45)cc3)c3ccccc3-c3ccc4c5ccccc5c5c(ccc6oc7ccccc7c65)c4c3)cc21 ; CC1(C)c2ccccc2-c2ccc(N(c3ccc(-c4ccc(-c5ccccc5)cc4)cc3)c3ccccc3-c3ccc4c5ccccc5c5c(ccc6oc7ccccc7c65)c4c3)cc21 ; CC1(C)c2ccccc2-c2ccc(N(c3cccc(-c4ccc(-c5ccccc5)cc4)c3)c3ccccc3-c3ccc4c5ccccc5c5c(ccc6oc7ccccc7c65)c4c3)cc21]

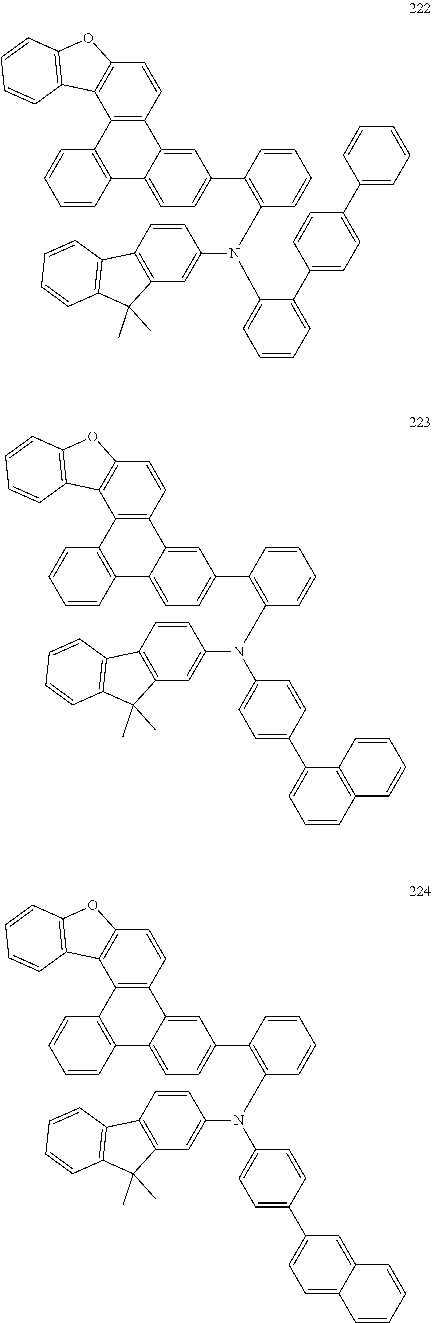 [SMILES: CC1(C)c2ccccc2-c2ccc(N(c3ccccc3-c3ccc(-c4ccccc4)cc3)c3ccccc3-c3ccc4c5ccccc5c5c(ccc6oc7ccccc7c65)c4c3)cc21 ; CC1(C)c2ccccc2-c2ccc(N(c3ccc(-c4cccc5ccccc45)cc3)c3ccccc3-c3ccc4c5ccccc5c5c(ccc6oc7ccccc7c65)c4c3)cc21 ; CC1(C)c2ccccc2-c2ccc(N(c3ccc(-c4ccc5ccccc5c4)cc3)c3ccccc3-c3ccc4c5ccccc5c5c(ccc6oc7ccccc7c65)c4c3)cc21]

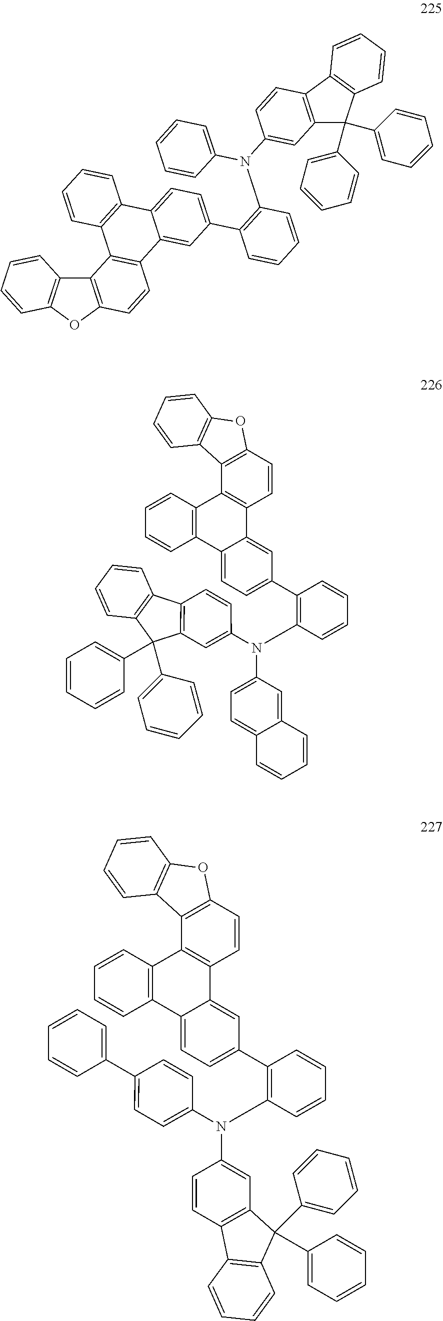 [SMILES: c1ccc(N(c2ccc3c(c2)C(c2ccccc2)(c2ccccc2)c2ccccc2-3)c2ccccc2-c2ccc3c4ccccc4c4c(ccc5oc6ccccc6c54)c3c2)cc1 ; c1ccc(C2(c3ccccc3)c3ccccc3-c3ccc(N(c4ccc5ccccc5c4)c4ccccc4-c4ccc5c6ccccc6c6c(ccc7oc8ccccc8c76)c5c4)cc32)cc1 ; c1ccc(-c2ccc(N(c3ccc4c(c3)C(c3ccccc3)(c3ccccc3)c3ccccc3-4)c3ccccc3-c3ccc4c5ccccc5c5c(ccc6oc7ccccc7c65)c4c3)cc2)cc1]

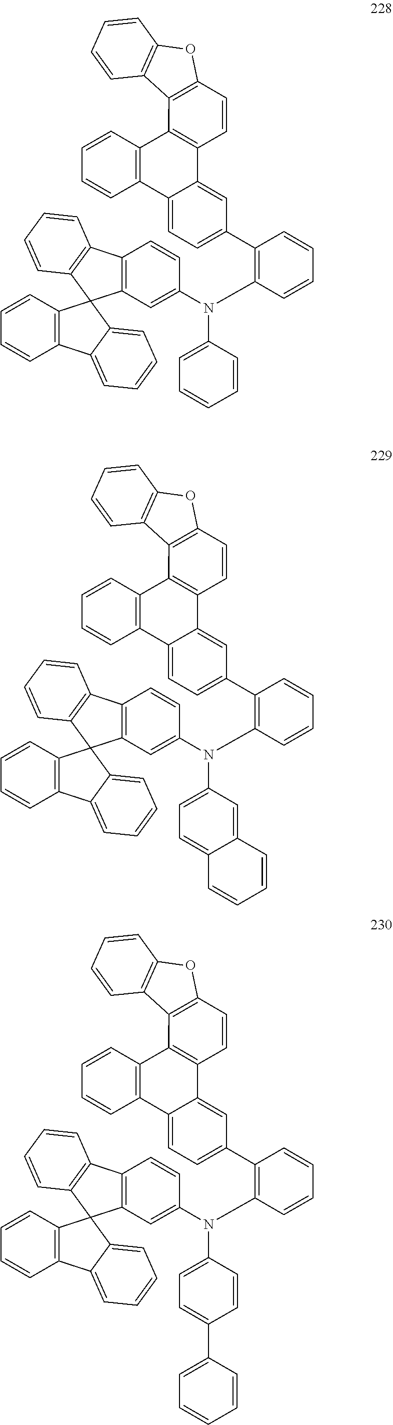 [SMILES: c1ccc(N(c2ccc3c(c2)C2(c4ccccc4-c4ccccc42)c2ccccc2-3)c2ccccc2-c2ccc3c4ccccc4c4c(ccc5oc6ccccc6c54)c3c2)cc1 ; c1ccc(N(c2ccc3c(c2)C2(c4ccccc4-c4ccccc42)c2ccccc2-3)c2ccc3ccccc3c2)c(-c2ccc3c4ccccc4c4c(ccc5oc6ccccc6c54)c3c2)c1 ; c1ccc(-c2ccc(N(c3ccc4c(c3)C3(c5ccccc5-c5ccccc53)c3ccccc3-4)c3ccccc3-c3ccc4c5ccccc5c5c(ccc6oc7ccccc7c65)c4c3)cc2)cc1]

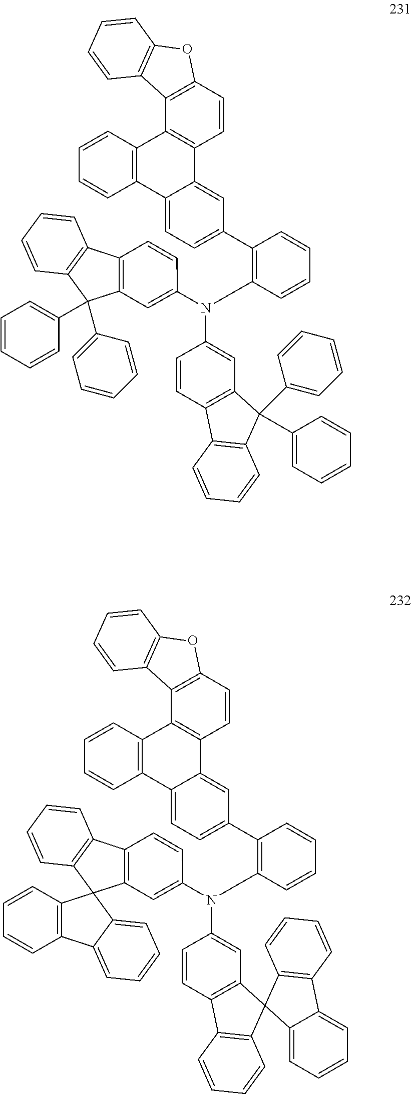 [SMILES: c1ccc(C2(c3ccccc3)c3ccccc3-c3ccc(N(c4ccc5c(c4)C(c4ccccc4)(c4ccccc4)c4ccccc4-5)c4ccccc4-c4ccc5c6ccccc6c6c(ccc7oc8ccccc8c76)c5c4)cc32)cc1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3c4ccccc4c4c(ccc5oc6ccccc6c54)c3c2)c1]

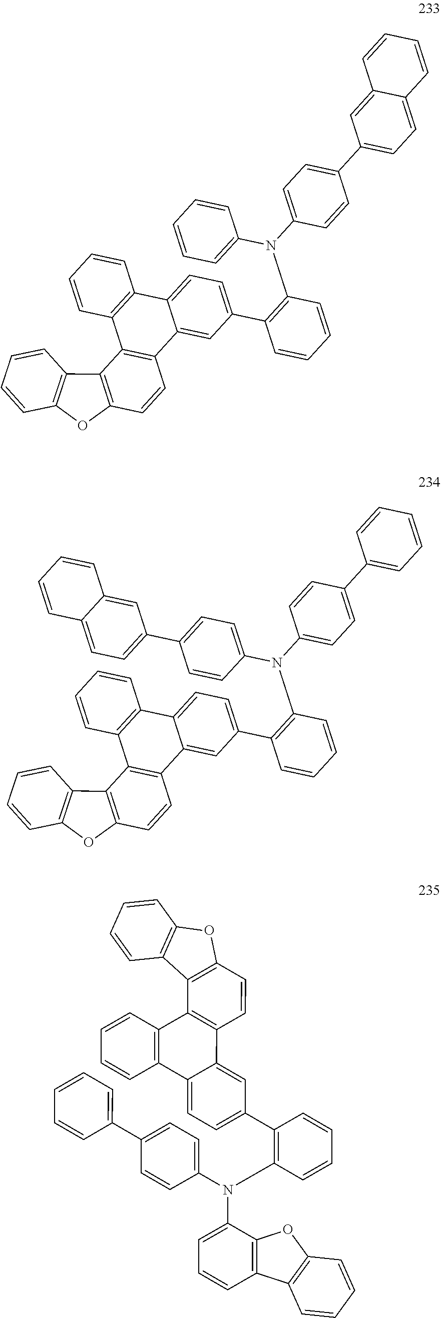 [SMILES: c1ccc(N(c2ccc(-c3ccc4ccccc4c3)cc2)c2ccccc2-c2ccc3c4ccccc4c4c(ccc5oc6ccccc6c54)c3c2)cc1 ; c1ccc(-c2ccc(N(c3ccc(-c4ccc5ccccc5c4)cc3)c3ccccc3-c3ccc4c5ccccc5c5c(ccc6oc7ccccc7c65)c4c3)cc2)cc1 ; c1ccc(-c2ccc(N(c3ccccc3-c3ccc4c5ccccc5c5c(ccc6oc7ccccc7c65)c4c3)c3cccc4c3oc3ccccc34)cc2)cc1]

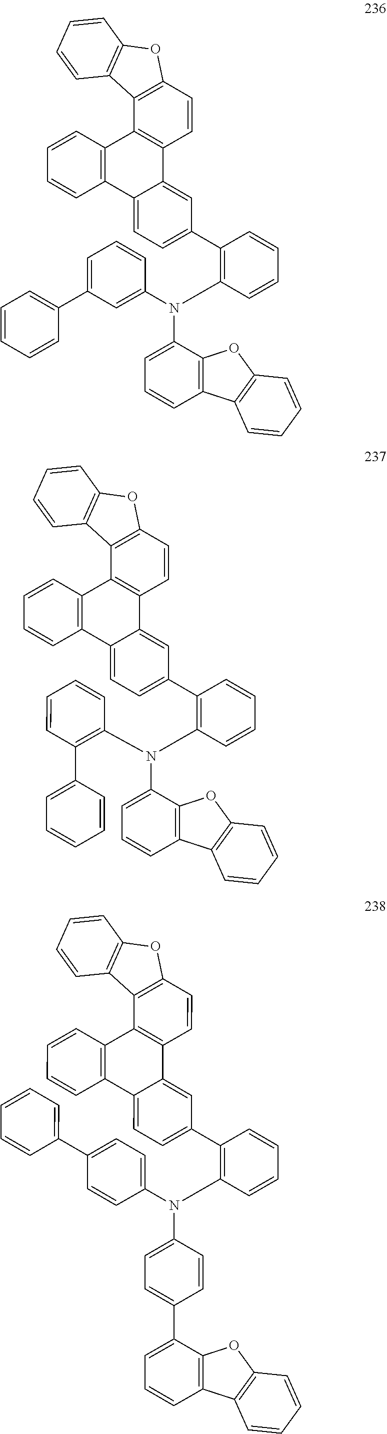 [SMILES: c1ccc(-c2cccc(N(c3ccccc3-c3ccc4c5ccccc5c5c(ccc6oc7ccccc7c65)c4c3)c3cccc4c3oc3ccccc34)c2)cc1 ; c1ccc(-c2ccccc2N(c2ccccc2-c2ccc3c4ccccc4c4c(ccc5oc6ccccc6c54)c3c2)c2cccc3c2oc2ccccc23)cc1 ; c1ccc(-c2ccc(N(c3ccc(-c4cccc5c4oc4ccccc45)cc3)c3ccccc3-c3ccc4c5ccccc5c5c(ccc6oc7ccccc7c65)c4c3)cc2)cc1]

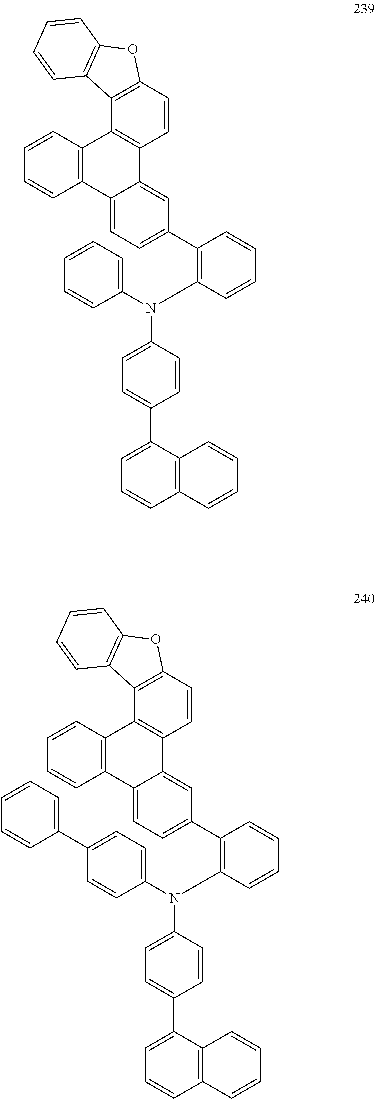 [SMILES: c1ccc(N(c2ccc(-c3cccc4ccccc34)cc2)c2ccccc2-c2ccc3c4ccccc4c4c(ccc5oc6ccccc6c54)c3c2)cc1 ; c1ccc(-c2ccc(N(c3ccc(-c4cccc5ccccc45)cc3)c3ccccc3-c3ccc4c5ccccc5c5c(ccc6oc7ccccc7c65)c4c3)cc2)cc1]

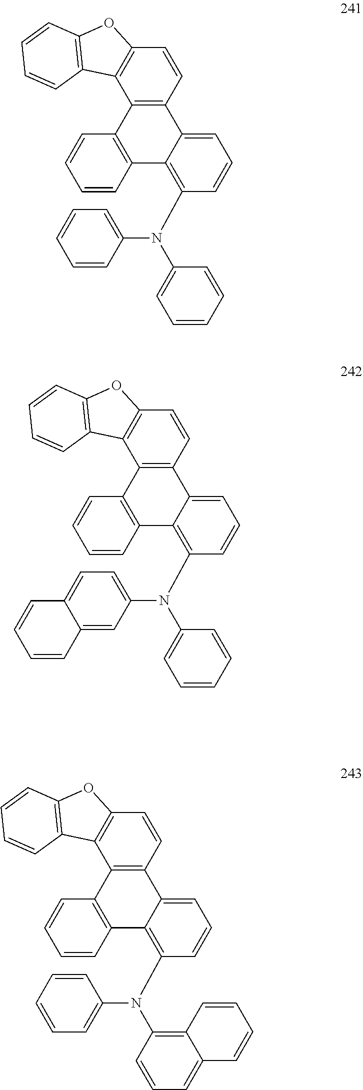 [SMILES: c1ccc(N(c2ccccc2)c2cccc3c4ccc5oc6ccccc6c5c4c4ccccc4c23)cc1 ; c1ccc(N(c2ccc3ccccc3c2)c2cccc3c4ccc5oc6ccccc6c5c4c4ccccc4c23)cc1 ; c1ccc(N(c2cccc3ccccc23)c2cccc3c4ccc5oc6ccccc6c5c4c4ccccc4c23)cc1]

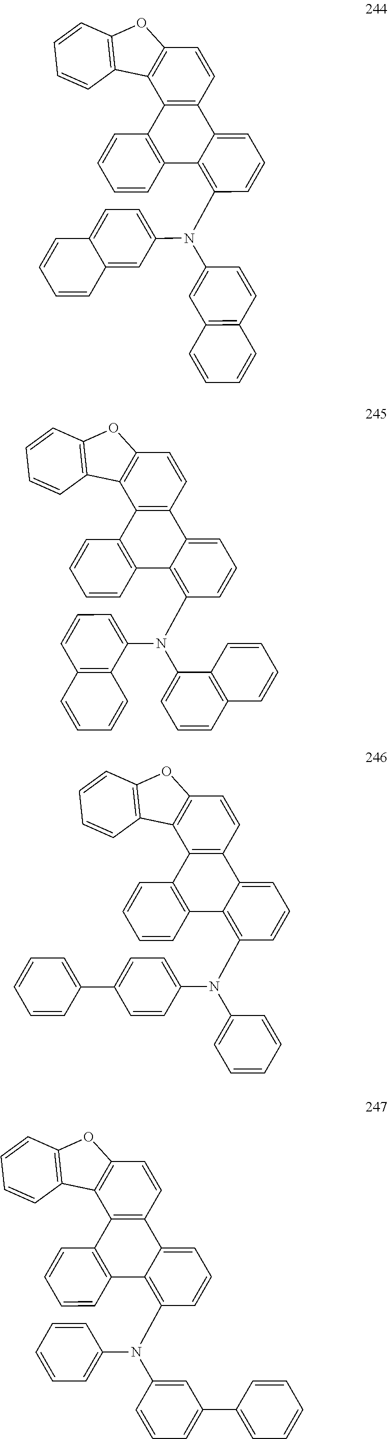

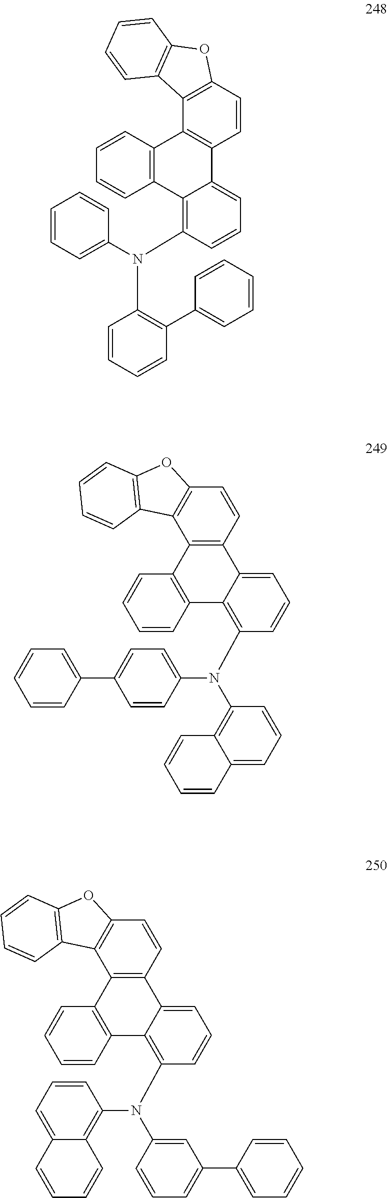 [SMILES: c1ccc(-c2ccccc2N(c2ccccc2)c2cccc3c4ccc5oc6ccccc6c5c4c4ccccc4c23)cc1 ; c1ccc(-c2ccc(N(c3cccc4ccccc34)c3cccc4c5ccc6oc7ccccc7c6c5c5ccccc5c34)cc2)cc1 ; c1ccc(-c2cccc(N(c3cccc4ccccc34)c3cccc4c5ccc6oc7ccccc7c6c5c5ccccc5c34)c2)cc1]

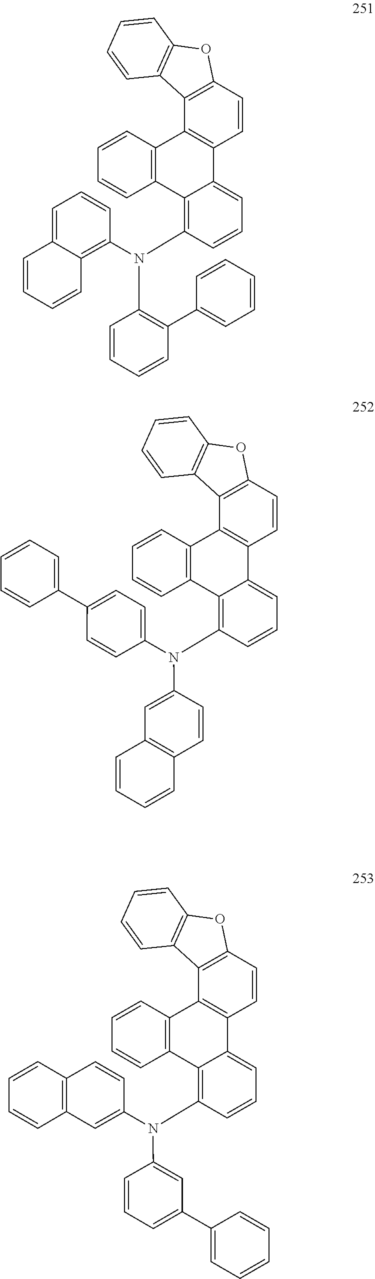 [SMILES: c1ccc(-c2ccccc2N(c2cccc3ccccc23)c2cccc3c4ccc5oc6ccccc6c5c4c4ccccc4c23)cc1 ; c1ccc(-c2ccc(N(c3ccc4ccccc4c3)c3cccc4c5ccc6oc7ccccc7c6c5c5ccccc5c34)cc2)cc1 ; c1ccc(-c2cccc(N(c3ccc4ccccc4c3)c3cccc4c5ccc6oc7ccccc7c6c5c5ccccc5c34)c2)cc1]

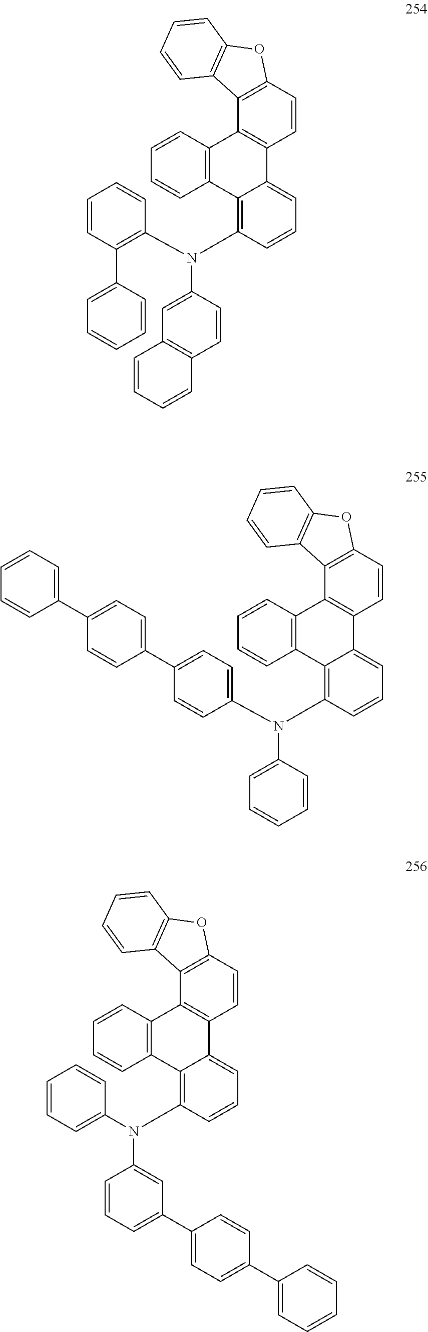 [SMILES: c1ccc(-c2ccccc2N(c2ccc3ccccc3c2)c2cccc3c4ccc5oc6ccccc6c5c4c4ccccc4c23)cc1 ; c1ccc(-c2ccc(-c3ccc(N(c4ccccc4)c4cccc5c6ccc7oc8ccccc8c7c6c6ccccc6c45)cc3)cc2)cc1 ; c1ccc(-c2ccc(-c3cccc(N(c4ccccc4)c4cccc5c6ccc7oc8ccccc8c7c6c6ccccc6c45)c3)cc2)cc1]

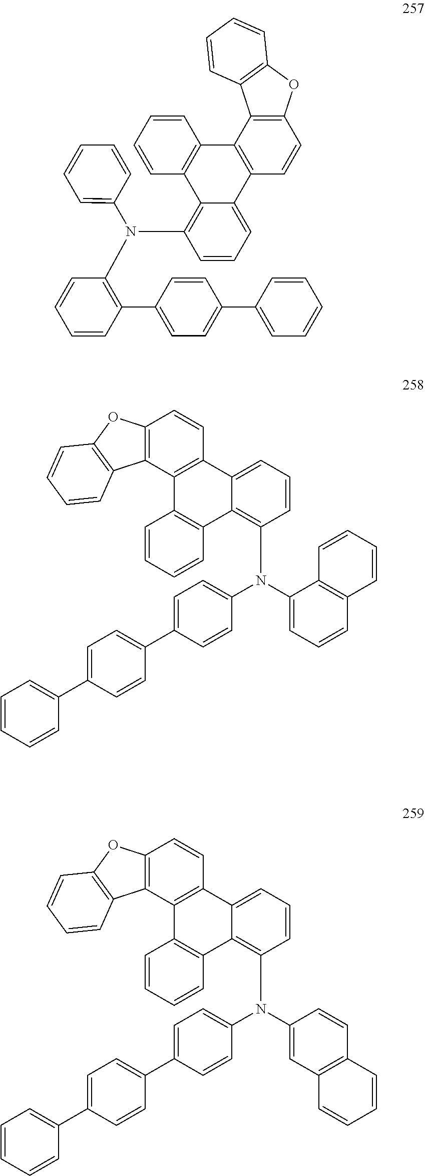 [SMILES: c1ccc(-c2ccc(-c3ccccc3N(c3ccccc3)c3cccc4c5ccc6oc7ccccc7c6c5c5ccccc5c34)cc2)cc1 ; c1ccc(-c2ccc(-c3ccc(N(c4cccc5ccccc45)c4cccc5c6ccc7oc8ccccc8c7c6c6ccccc6c45)cc3)cc2)cc1 ; c1ccc(-c2ccc(-c3ccc(N(c4ccc5ccccc5c4)c4cccc5c6ccc7oc8ccccc8c7c6c6ccccc6c45)cc3)cc2)cc1]

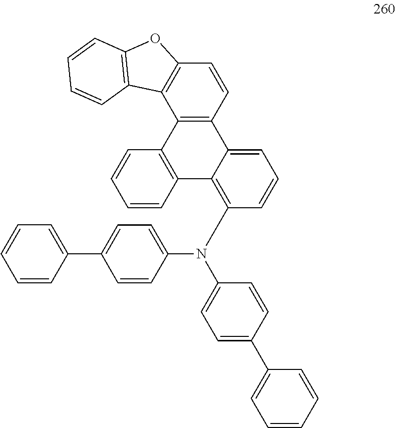 [SMILES: c1ccc(-c2ccc(N(c3ccc(-c4ccccc4)cc3)c3cccc4c5ccc6oc7ccccc7c6c5c5ccccc5c34)cc2)cc1]

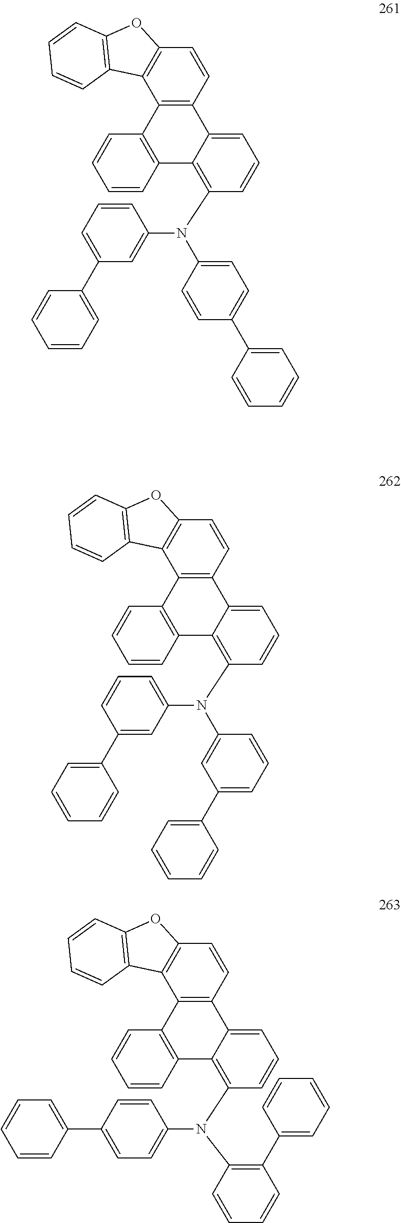

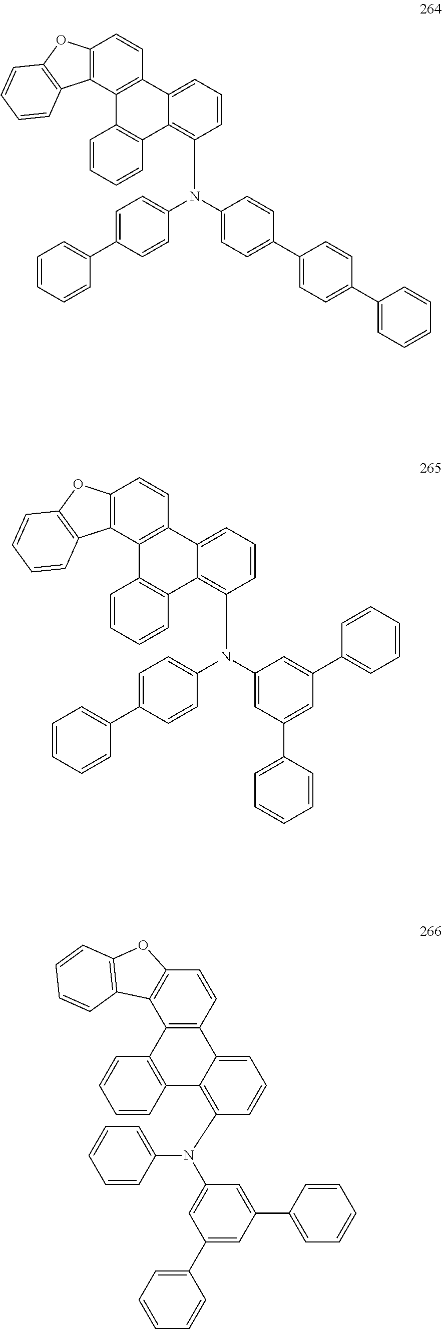 [SMILES: c1ccc(-c2ccc(-c3ccc(N(c4ccc(-c5ccccc5)cc4)c4cccc5c6ccc7oc8ccccc8c7c6c6ccccc6c45)cc3)cc2)cc1 ; c1ccc(-c2ccc(N(c3cc(-c4ccccc4)cc(-c4ccccc4)c3)c3cccc4c5ccc6oc7ccccc7c6c5c5ccccc5c34)cc2)cc1 ; c1ccc(-c2cc(-c3ccccc3)cc(N(c3ccccc3)c3cccc4c5ccc6oc7ccccc7c6c5c5ccccc5c34)c2)cc1]

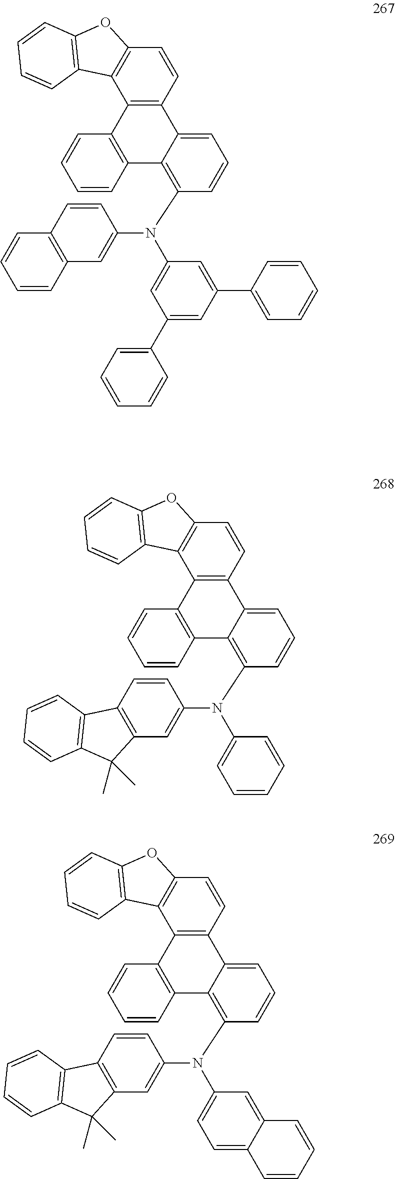 [SMILES: c1ccc(-c2cc(-c3ccccc3)cc(N(c3ccc4ccccc4c3)c3cccc4c5ccc6oc7ccccc7c6c5c5ccccc5c34)c2)cc1 ; CC1(C)c2ccccc2-c2ccc(N(c3ccccc3)c3cccc4c5ccc6oc7ccccc7c6c5c5ccccc5c34)cc21 ; CC1(C)c2ccccc2-c2ccc(N(c3ccc4ccccc4c3)c3cccc4c5ccc6oc7ccccc7c6c5c5ccccc5c34)cc21]

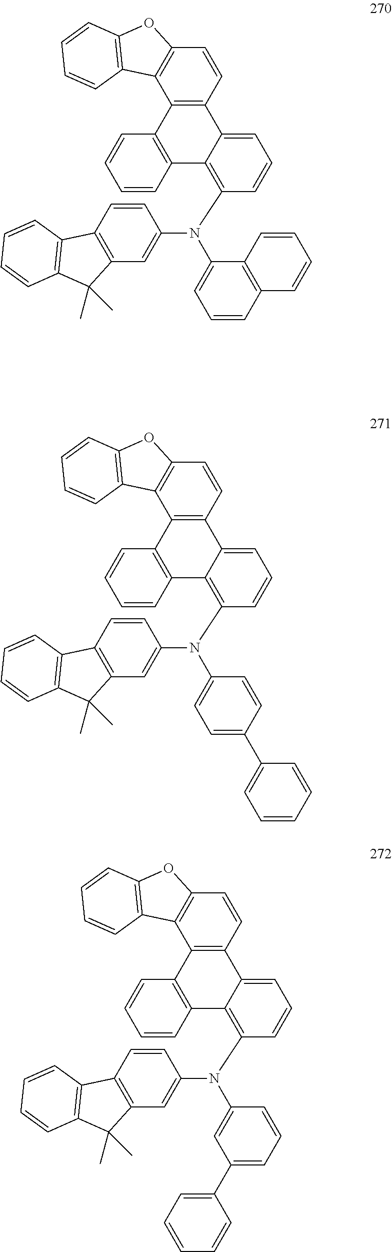 [SMILES: CC1(C)c2ccccc2-c2ccc(N(c3cccc4ccccc34)c3cccc4c5ccc6oc7ccccc7c6c5c5ccccc5c34)cc21 ; CC1(C)c2ccccc2-c2ccc(N(c3ccc(-c4ccccc4)cc3)c3cccc4c5ccc6oc7ccccc7c6c5c5ccccc5c34)cc21 ; CC1(C)c2ccccc2-c2ccc(N(c3cccc(-c4ccccc4)c3)c3cccc4c5ccc6oc7ccccc7c6c5c5ccccc5c34)cc21]

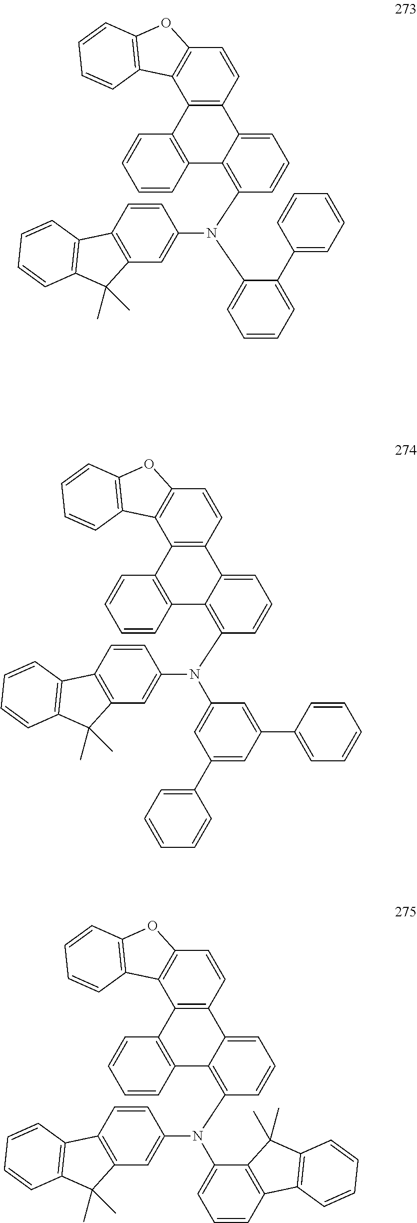 [SMILES: CC1(C)c2ccccc2-c2ccc(N(c3ccccc3-c3ccccc3)c3cccc4c5ccc6oc7ccccc7c6c5c5ccccc5c34)cc21 ; CC1(C)c2ccccc2-c2ccc(N(c3cc(-c4ccccc4)cc(-c4ccccc4)c3)c3cccc4c5ccc6oc7ccccc7c6c5c5ccccc5c34)cc21 ; CC1(C)c2ccccc2-c2ccc(N(c3cccc4c3C(C)(C)c3ccccc3-4)c3cccc4c5ccc6oc7ccccc7c6c5c5ccccc5c34)cc21]

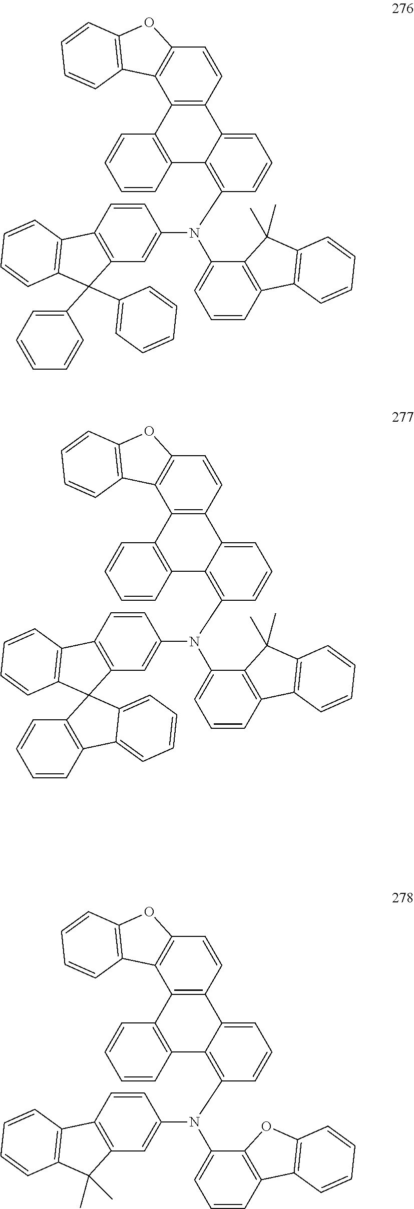 [SMILES: CC1(C)c2ccccc2-c2cccc(N(c3ccc4c(c3)C(c3ccccc3)(c3ccccc3)c3ccccc3-4)c3cccc4c5ccc6oc7ccccc7c6c5c5ccccc5c34)c21 ; CC1(C)c2ccccc2-c2cccc(N(c3ccc4c(c3)C3(c5ccccc5-c5ccccc53)c3ccccc3-4)c3cccc4c5ccc6oc7ccccc7c6c5c5ccccc5c34)c21 ; CC1(C)c2ccccc2-c2ccc(N(c3cccc4c3oc3ccccc34)c3cccc4c5ccc6oc7ccccc7c6c5c5ccccc5c34)cc21]

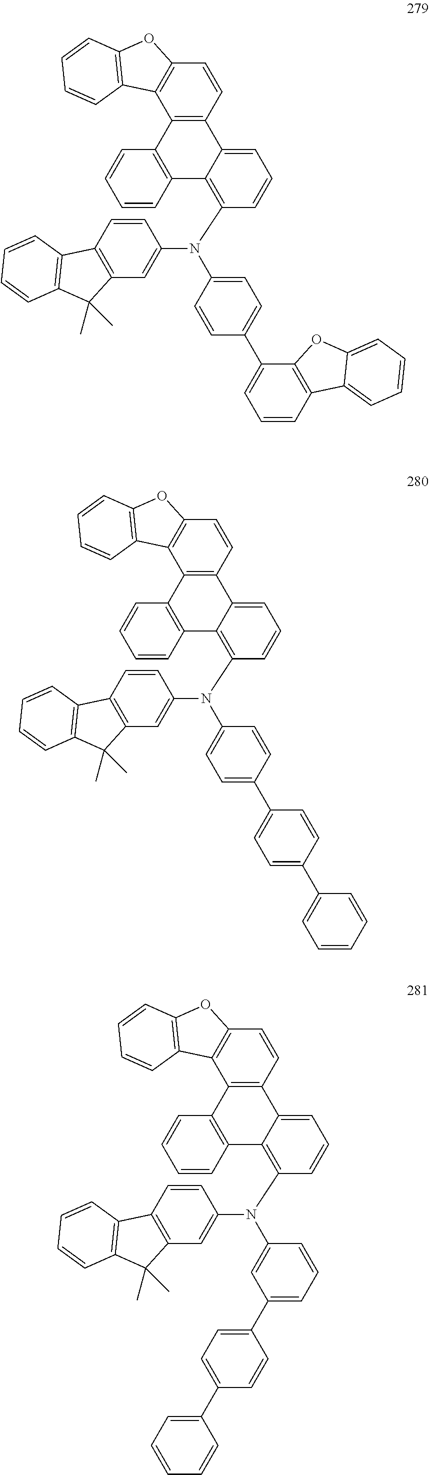 [SMILES: CC1(C)c2ccccc2-c2ccc(N(c3ccc(-c4cccc5c4oc4ccccc45)cc3)c3cccc4c5ccc6oc7ccccc7c6c5c5ccccc5c34)cc21 ; CC1(C)c2ccccc2-c2ccc(N(c3ccc(-c4ccc(-c5ccccc5)cc4)cc3)c3cccc4c5ccc6oc7ccccc7c6c5c5ccccc5c34)cc21 ; CC1(C)c2ccccc2-c2ccc(N(c3cccc(-c4ccc(-c5ccccc5)cc4)c3)c3cccc4c5ccc6oc7ccccc7c6c5c5ccccc5c34)cc21]

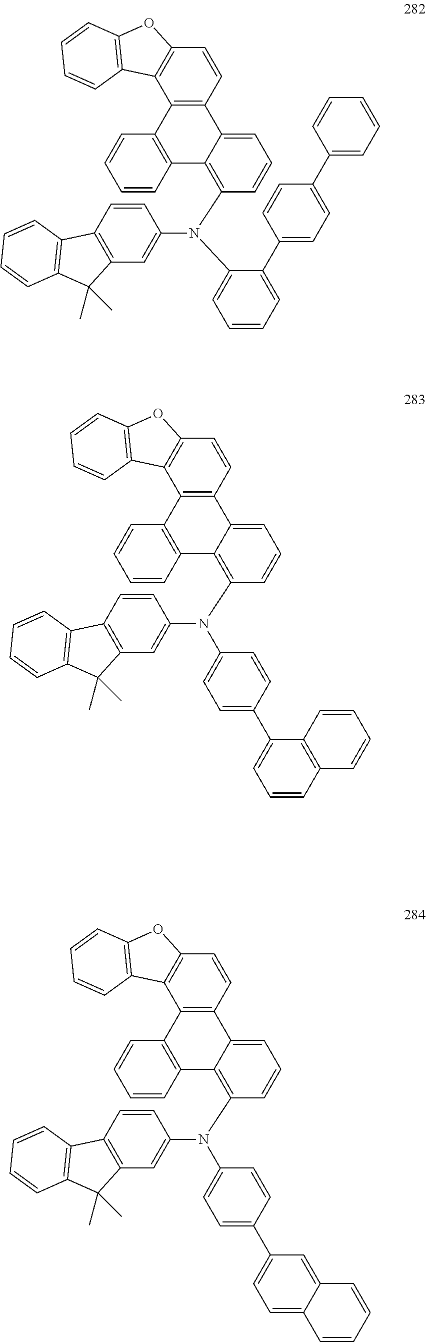 [SMILES: CC1(C)c2ccccc2-c2ccc(N(c3ccccc3-c3ccc(-c4ccccc4)cc3)c3cccc4c5ccc6oc7ccccc7c6c5c5ccccc5c34)cc21 ; CC1(C)c2ccccc2-c2ccc(N(c3ccc(-c4cccc5ccccc45)cc3)c3cccc4c5ccc6oc7ccccc7c6c5c5ccccc5c34)cc21 ; CC1(C)c2ccccc2-c2ccc(N(c3ccc(-c4ccc5ccccc5c4)cc3)c3cccc4c5ccc6oc7ccccc7c6c5c5ccccc5c34)cc21]

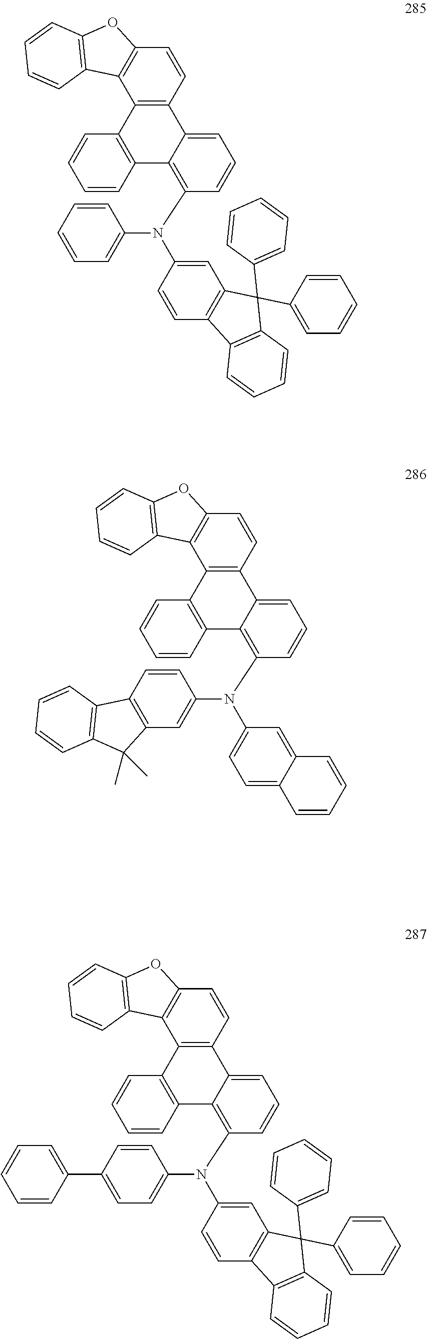 [SMILES: c1ccc(N(c2ccc3c(c2)C(c2ccccc2)(c2ccccc2)c2ccccc2-3)c2cccc3c4ccc5oc6ccccc6c5c4c4ccccc4c23)cc1 ; CC1(C)c2ccccc2-c2ccc(N(c3ccc4ccccc4c3)c3cccc4c5ccc6oc7ccccc7c6c5c5ccccc5c34)cc21 ; c1ccc(-c2ccc(N(c3ccc4c(c3)C(c3ccccc3)(c3ccccc3)c3ccccc3-4)c3cccc4c5ccc6oc7ccccc7c6c5c5ccccc5c34)cc2)cc1]

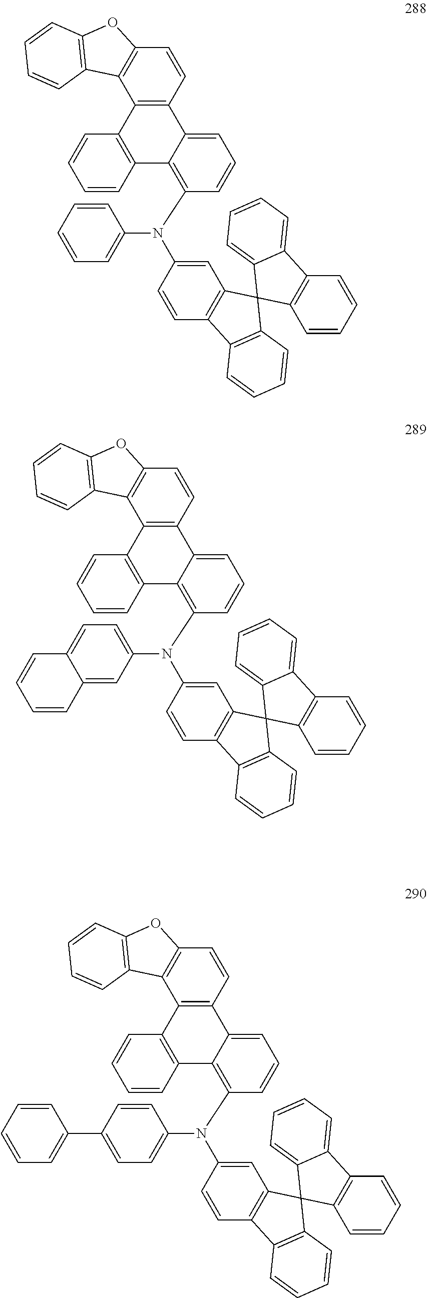 [SMILES: c1ccc(N(c2ccc3c(c2)C2(c4ccccc4-c4ccccc42)c2ccccc2-3)c2cccc3c4ccc5oc6ccccc6c5c4c4ccccc4c23)cc1 ; c1ccc2c(c1)-c1ccccc1C21c2ccccc2-c2ccc(N(c3ccc4ccccc4c3)c3cccc4c5ccc6oc7ccccc7c6c5c5ccccc5c34)cc21 ; c1ccc(-c2ccc(N(c3ccc4c(c3)C3(c5ccccc5-c5ccccc53)c3ccccc3-4)c3cccc4c5ccc6oc7ccccc7c6c5c5ccccc5c34)cc2)cc1]

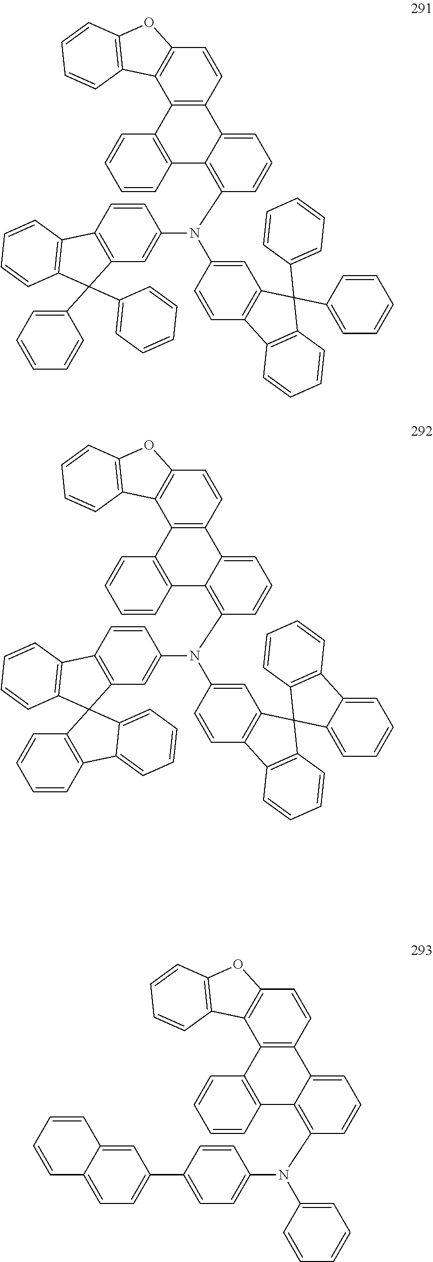 [SMILES: c1ccc(C2(c3ccccc3)c3ccccc3-c3ccc(N(c4ccc5c(c4)C(c4ccccc4)(c4ccccc4)c4ccccc4-5)c4cccc5c6ccc7oc8ccccc8c7c6c6ccccc6c45)cc32)cc1 ; c1ccc2c(c1)-c1ccccc1C21c2ccccc2-c2ccc(N(c3ccc4c(c3)C3(c5ccccc5-c5ccccc53)c3ccccc3-4)c3cccc4c5ccc6oc7ccccc7c6c5c5ccccc5c34)cc21 ; c1ccc(N(c2ccc(-c3ccc4ccccc4c3)cc2)c2cccc3c4ccc5oc6ccccc6c5c4c4ccccc4c23)cc1]

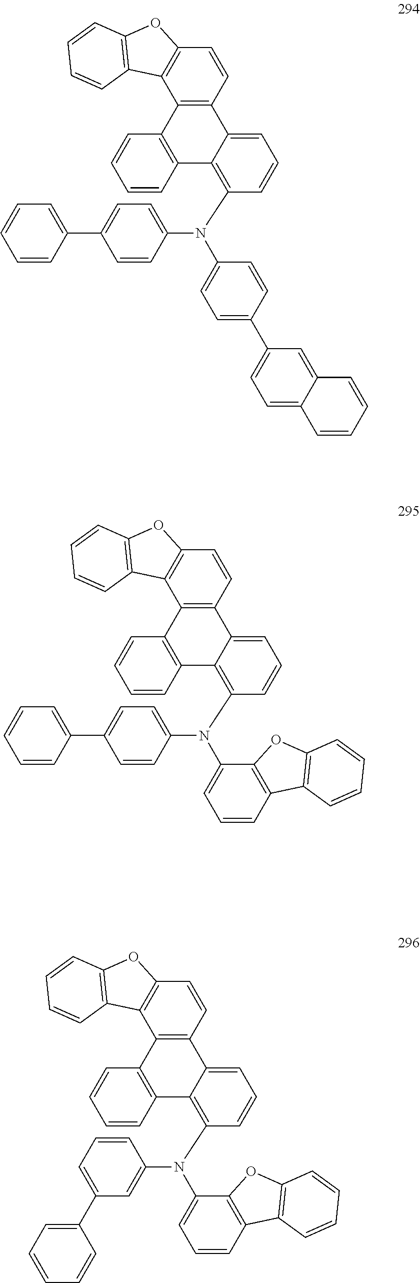 [SMILES: c1ccc(-c2ccc(N(c3ccc(-c4ccc5ccccc5c4)cc3)c3cccc4c5ccc6oc7ccccc7c6c5c5ccccc5c34)cc2)cc1 ; c1ccc(-c2ccc(N(c3cccc4c3oc3ccccc34)c3cccc4c5ccc6oc7ccccc7c6c5c5ccccc5c34)cc2)cc1 ; c1ccc(-c2cccc(N(c3cccc4c3oc3ccccc34)c3cccc4c5ccc6oc7ccccc7c6c5c5ccccc5c34)c2)cc1]

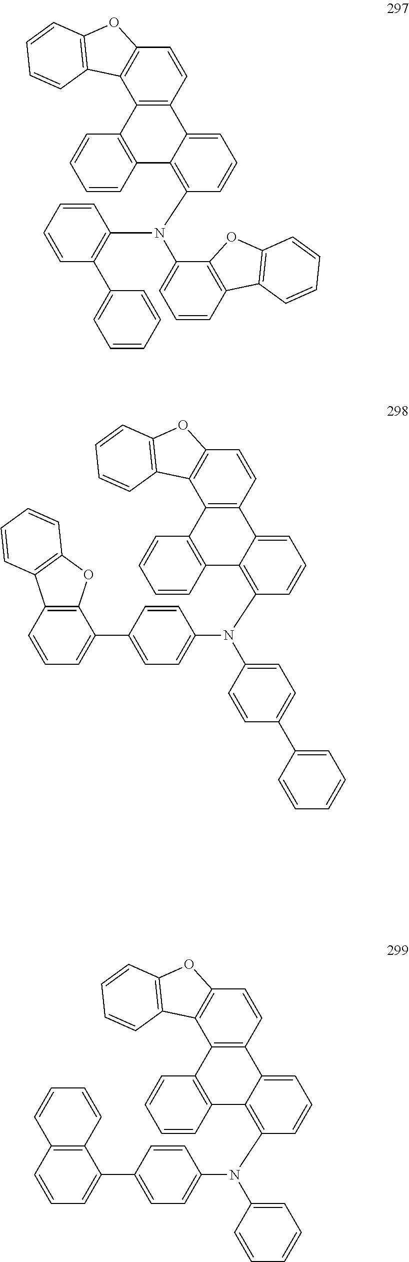 [SMILES: c1ccc(-c2ccccc2N(c2cccc3c2oc2ccccc23)c2cccc3c4ccc5oc6ccccc6c5c4c4ccccc4c23)cc1 ; c1ccc(-c2ccc(N(c3ccc(-c4cccc5c4oc4ccccc45)cc3)c3cccc4c5ccc6oc7ccccc7c6c5c5ccccc5c34)cc2)cc1 ; c1ccc(N(c2ccc(-c3cccc4ccccc34)cc2)c2cccc3c4ccc5oc6ccccc6c5c4c4ccccc4c23)cc1]

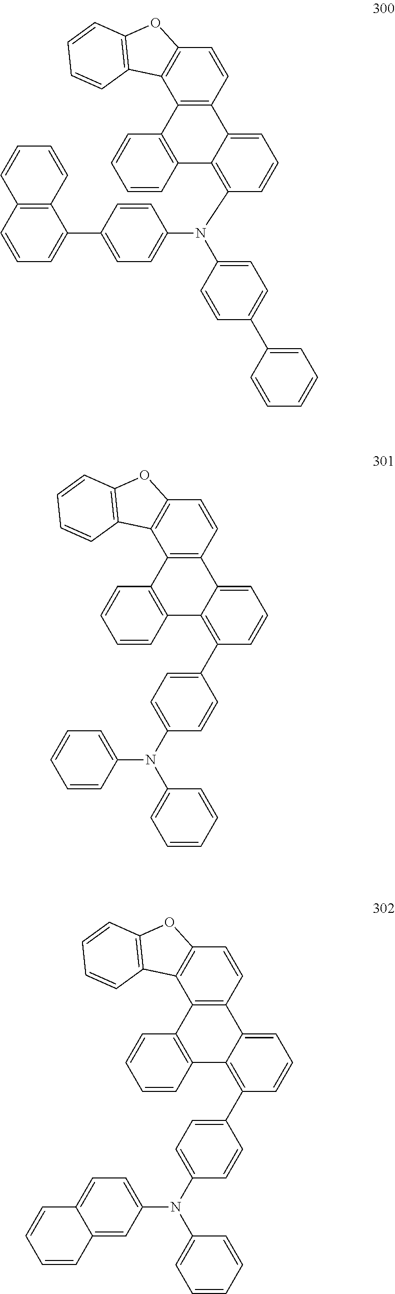 [SMILES: c1ccc(-c2ccc(N(c3ccc(-c4cccc5ccccc45)cc3)c3cccc4c5ccc6oc7ccccc7c6c5c5ccccc5c34)cc2)cc1 ; c1ccc(N(c2ccccc2)c2ccc(-c3cccc4c5ccc6oc7ccccc7c6c5c5ccccc5c34)cc2)cc1 ; c1ccc(N(c2ccc(-c3cccc4c5ccc6oc7ccccc7c6c5c5ccccc5c34)cc2)c2ccc3ccccc3c2)cc1]

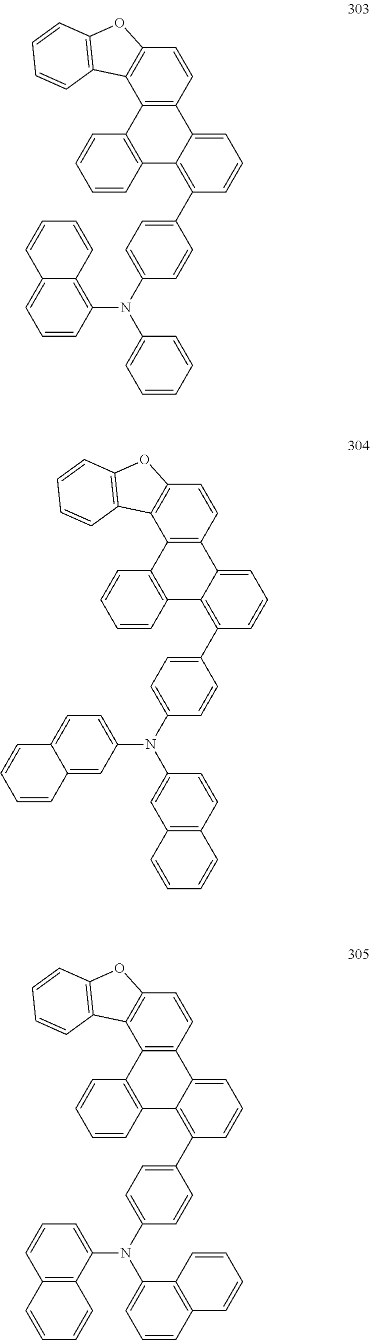 [SMILES: c1ccc(N(c2ccc(-c3cccc4c5ccc6oc7ccccc7c6c5c5ccccc5c34)cc2)c2cccc3ccccc23)cc1 ; c1ccc2cc(N(c3ccc(-c4cccc5c6ccc7oc8ccccc8c7c6c6ccccc6c45)cc3)c3ccc4ccccc4c3)ccc2c1 ; c1ccc2c(N(c3ccc(-c4cccc5c6ccc7oc8ccccc8c7c6c6ccccc6c45)cc3)c3cccc4ccccc34)cccc2c1]

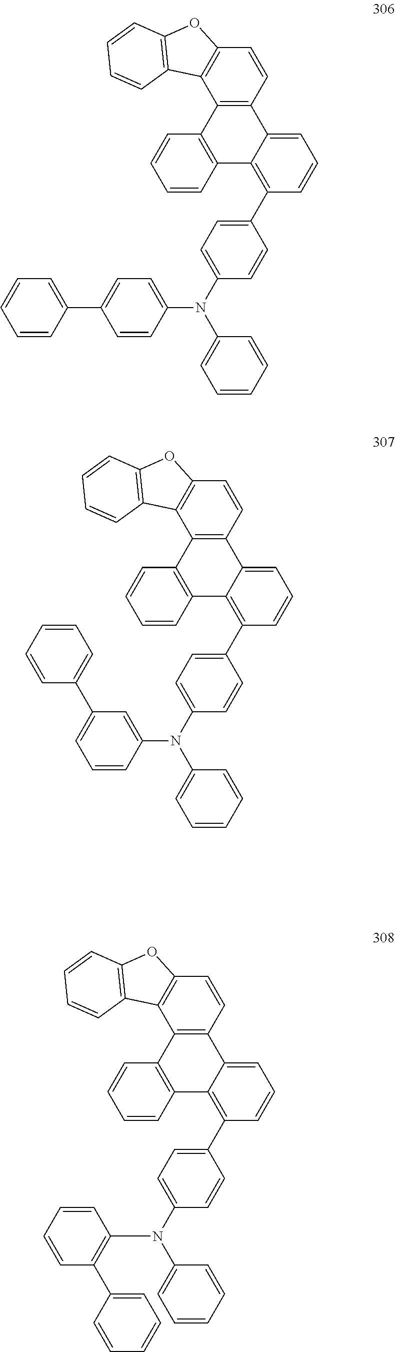 [SMILES: c1ccc(-c2ccc(N(c3ccccc3)c3ccc(-c4cccc5c6ccc7oc8ccccc8c7c6c6ccccc6c45)cc3)cc2)cc1 ; c1ccc(-c2cccc(N(c3ccccc3)c3ccc(-c4cccc5c6ccc7oc8ccccc8c7c6c6ccccc6c45)cc3)c2)cc1 ; c1ccc(-c2ccccc2N(c2ccccc2)c2ccc(-c3cccc4c5ccc6oc7ccccc7c6c5c5ccccc5c34)cc2)cc1]

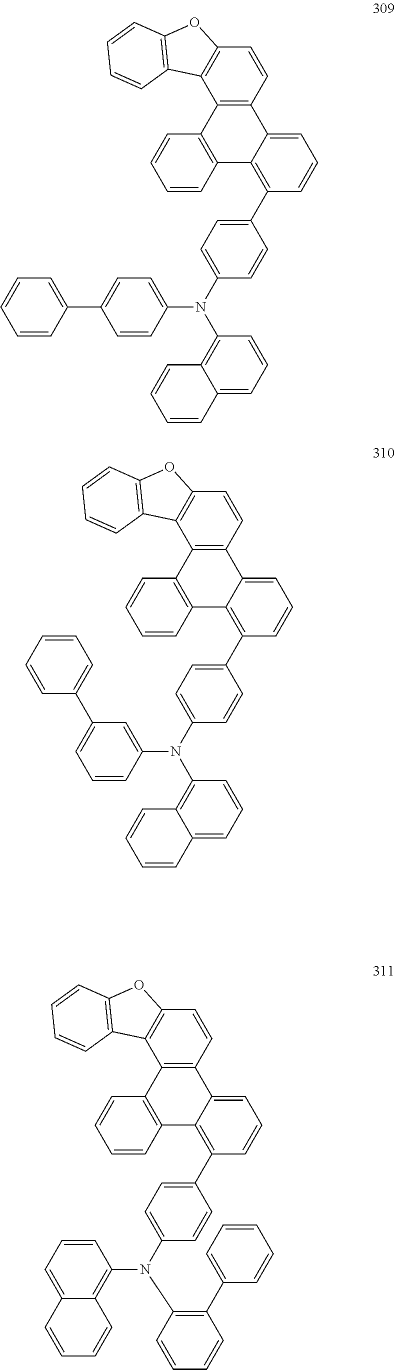

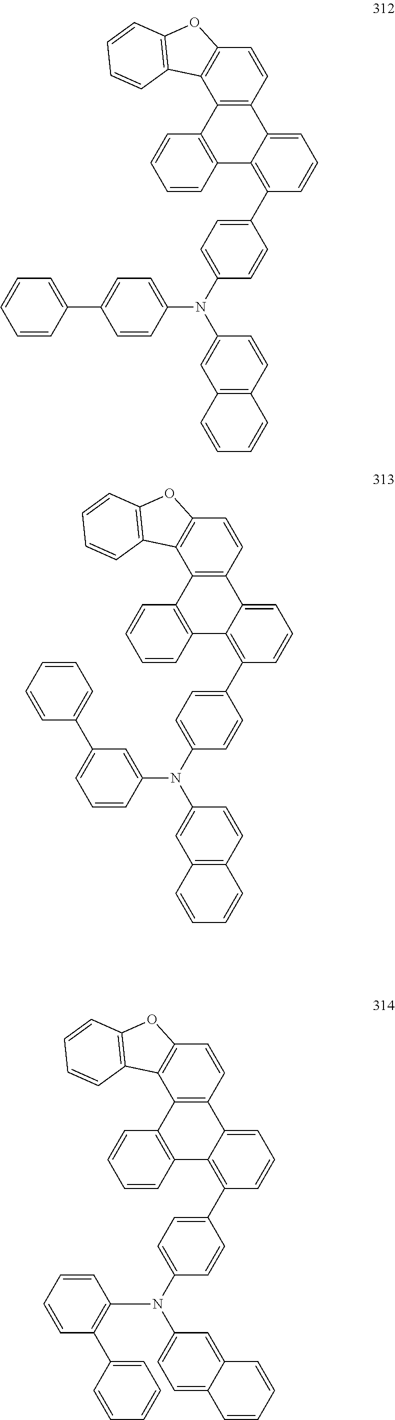 [SMILES: c1ccc(-c2ccc(N(c3ccc(-c4cccc5c6ccc7oc8ccccc8c7c6c6ccccc6c45)cc3)c3ccc4ccccc4c3)cc2)cc1 ; c1ccc(-c2cccc(N(c3ccc(-c4cccc5c6ccc7oc8ccccc8c7c6c6ccccc6c45)cc3)c3ccc4ccccc4c3)c2)cc1 ; c1ccc(-c2ccccc2N(c2ccc(-c3cccc4c5ccc6oc7ccccc7c6c5c5ccccc5c34)cc2)c2ccc3ccccc3c2)cc1]

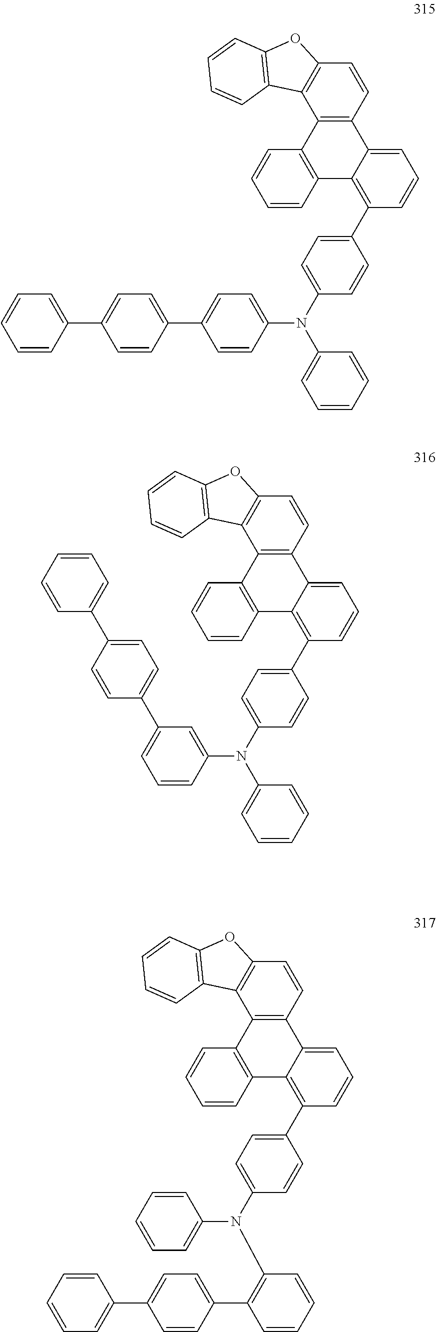 [SMILES: c1ccc(-c2ccc(-c3ccc(N(c4ccccc4)c4ccc(-c5cccc6c7ccc8oc9ccccc9c8c7c7ccccc7c56)cc4)cc3)cc2)cc1 ; c1ccc(-c2ccc(-c3cccc(N(c4ccccc4)c4ccc(-c5cccc6c7ccc8oc9ccccc9c8c7c7ccccc7c56)cc4)c3)cc2)cc1 ; c1ccc(-c2ccc(-c3ccccc3N(c3ccccc3)c3ccc(-c4cccc5c6ccc7oc8ccccc8c7c6c6ccccc6c45)cc3)cc2)cc1]

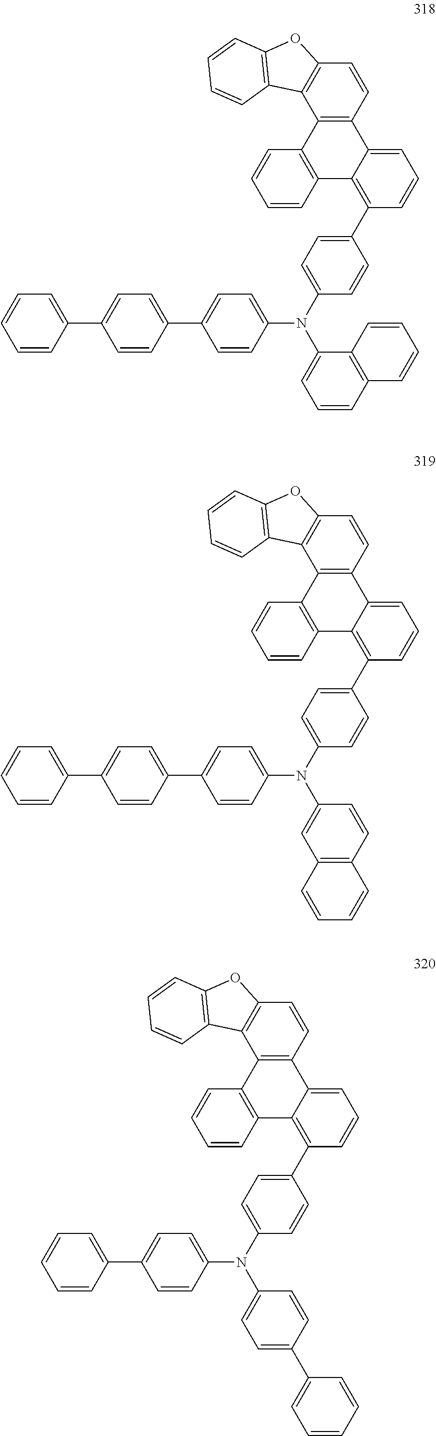 [SMILES: c1ccc(-c2ccc(-c3ccc(N(c4ccc(-c5cccc6c7ccc8oc9ccccc9c8c7c7ccccc7c56)cc4)c4cccc5ccccc45)cc3)cc2)cc1 ; c1ccc(-c2ccc(-c3ccc(N(c4ccc(-c5cccc6c7ccc8oc9ccccc9c8c7c7ccccc7c56)cc4)c4ccc5ccccc5c4)cc3)cc2)cc1 ; c1ccc(-c2ccc(N(c3ccc(-c4ccccc4)cc3)c3ccc(-c4cccc5c6ccc7oc8ccccc8c7c6c6ccccc6c45)cc3)cc2)cc1]

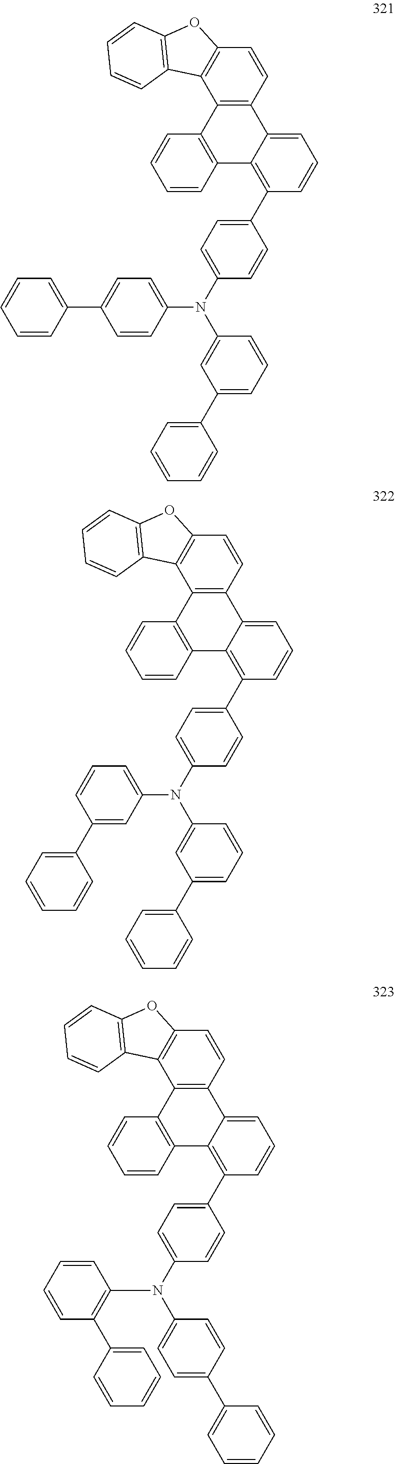

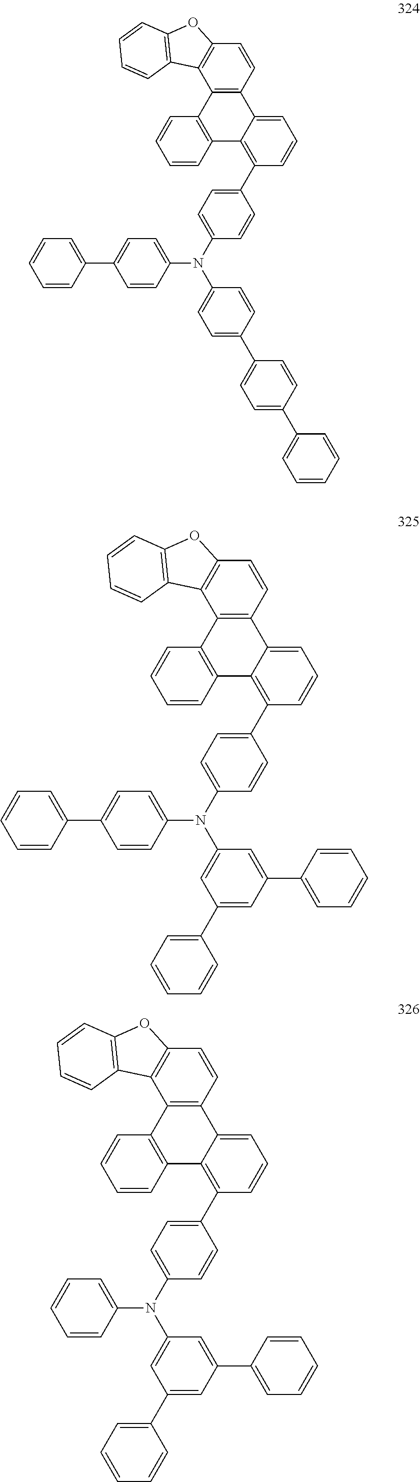

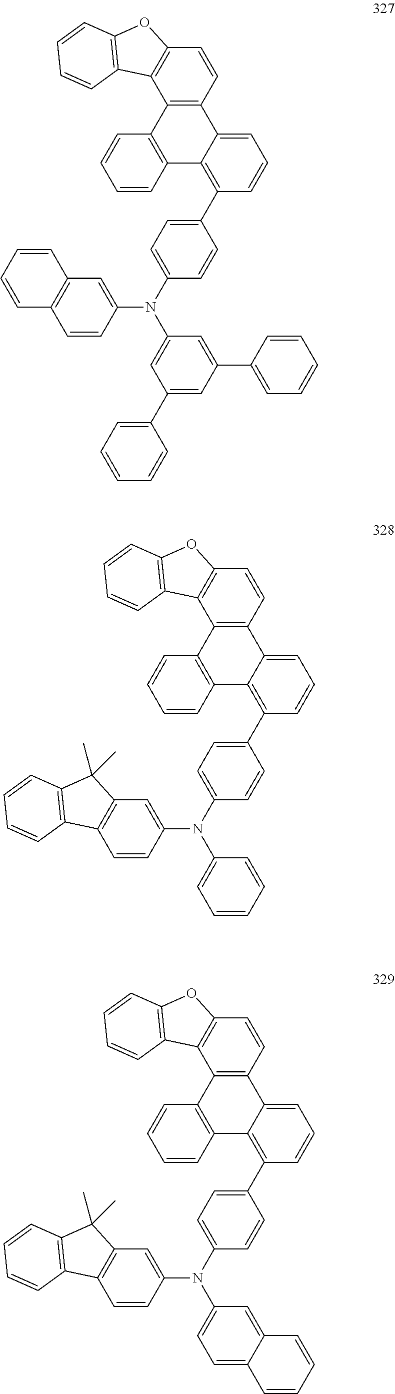 [SMILES: c1ccc(-c2cc(-c3ccccc3)cc(N(c3ccc(-c4cccc5c6ccc7oc8ccccc8c7c6c6ccccc6c45)cc3)c3ccc4ccccc4c3)c2)cc1 ; CC1(C)c2ccccc2-c2ccc(N(c3ccccc3)c3ccc(-c4cccc5c6ccc7oc8ccccc8c7c6c6ccccc6c45)cc3)cc21 ; CC1(C)c2ccccc2-c2ccc(N(c3ccc(-c4cccc5c6ccc7oc8ccccc8c7c6c6ccccc6c45)cc3)c3ccc4ccccc4c3)cc21]

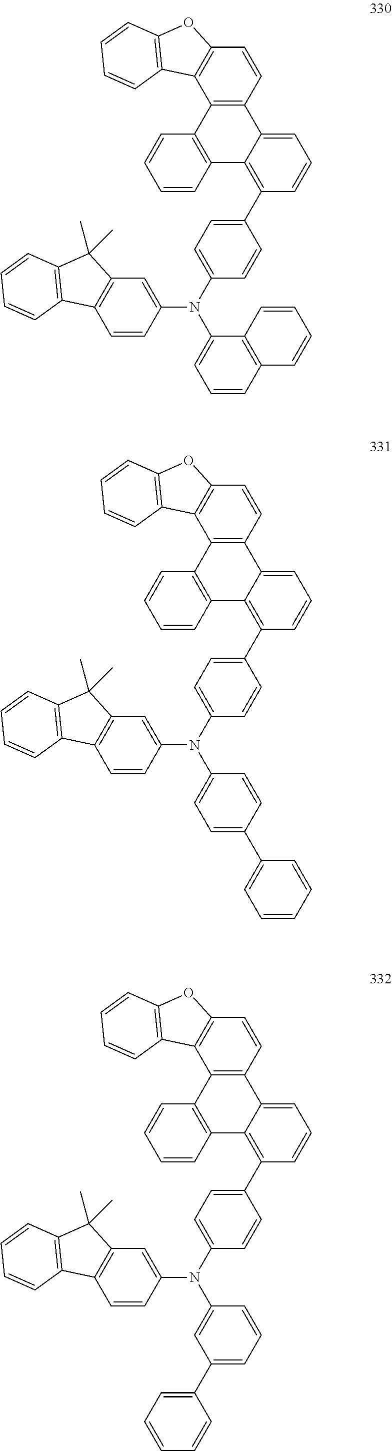 [SMILES: CC1(C)c2ccccc2-c2ccc(N(c3ccc(-c4cccc5c6ccc7oc8ccccc8c7c6c6ccccc6c45)cc3)c3cccc4ccccc34)cc21 ; CC1(C)c2ccccc2-c2ccc(N(c3ccc(-c4ccccc4)cc3)c3ccc(-c4cccc5c6ccc7oc8ccccc8c7c6c6ccccc6c45)cc3)cc21 ; CC1(C)c2ccccc2-c2ccc(N(c3ccc(-c4cccc5c6ccc7oc8ccccc8c7c6c6ccccc6c45)cc3)c3cccc(-c4ccccc4)c3)cc21]

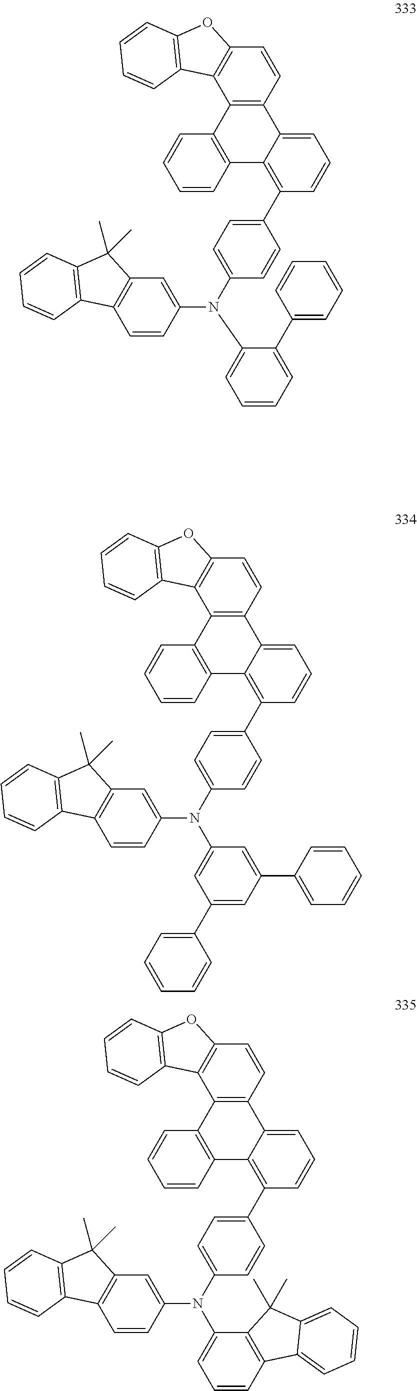

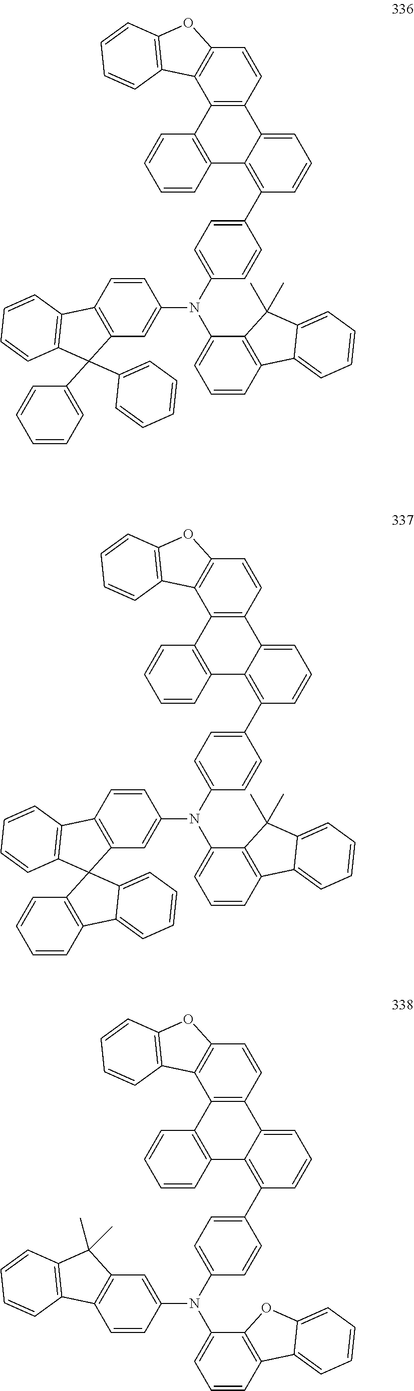 [SMILES: CC1(C)c2ccccc2-c2cccc(N(c3ccc(-c4cccc5c6ccc7oc8ccccc8c7c6c6ccccc6c45)cc3)c3ccc4c(c3)C(c3ccccc3)(c3ccccc3)c3ccccc3-4)c21 ; CC1(C)c2ccccc2-c2cccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)c21 ; CC1(C)c2ccccc2-c2ccc(N(c3ccc(-c4cccc5c6ccc7oc8ccccc8c7c6c6ccccc6c45)cc3)c3cccc4c3oc3ccccc34)cc21]

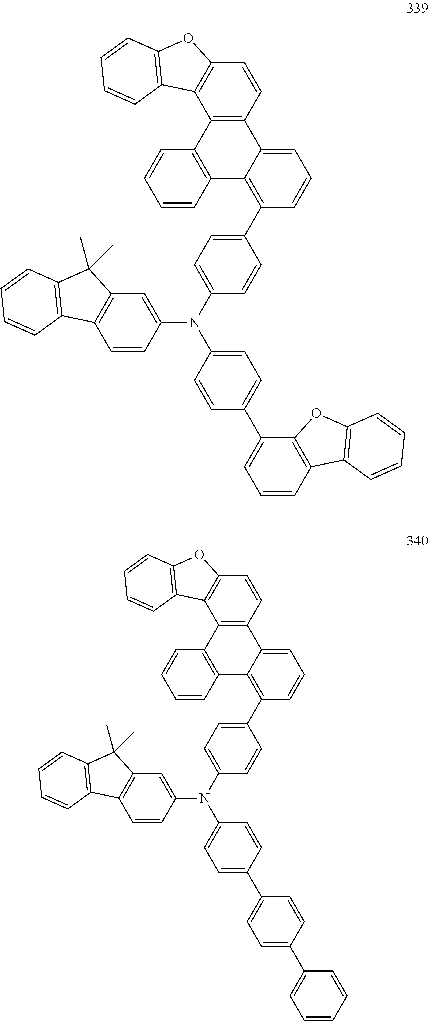 [SMILES: CC1(C)c2ccccc2-c2ccc(N(c3ccc(-c4cccc5c4oc4ccccc45)cc3)c3ccc(-c4cccc5c6ccc7oc8ccccc8c7c6c6ccccc6c45)cc3)cc21 ; CC1(C)c2ccccc2-c2ccc(N(c3ccc(-c4ccc(-c5ccccc5)cc4)cc3)c3ccc(-c4cccc5c6ccc7oc8ccccc8c7c6c6ccccc6c45)cc3)cc21]

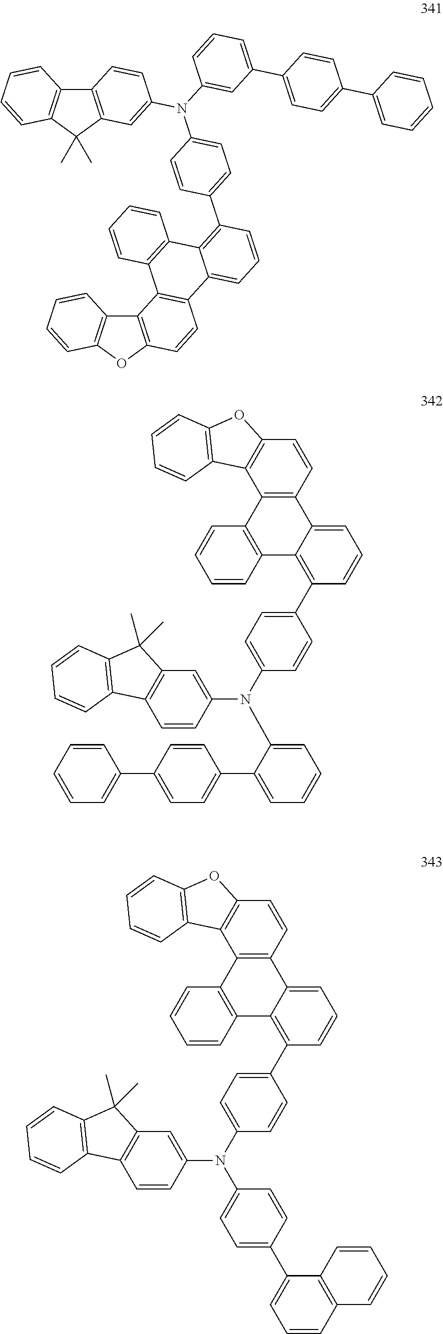 [SMILES: CC1(C)c2ccccc2-c2ccc(N(c3ccc(-c4cccc5c6ccc7oc8ccccc8c7c6c6ccccc6c45)cc3)c3cccc(-c4ccc(-c5ccccc5)cc4)c3)cc21 ; CC1(C)c2ccccc2-c2ccc(N(c3ccc(-c4cccc5c6ccc7oc8ccccc8c7c6c6ccccc6c45)cc3)c3ccccc3-c3ccc(-c4ccccc4)cc3)cc21 ; CC1(C)c2ccccc2-c2ccc(N(c3ccc(-c4cccc5ccccc45)cc3)c3ccc(-c4cccc5c6ccc7oc8ccccc8c7c6c6ccccc6c45)cc3)cc21]

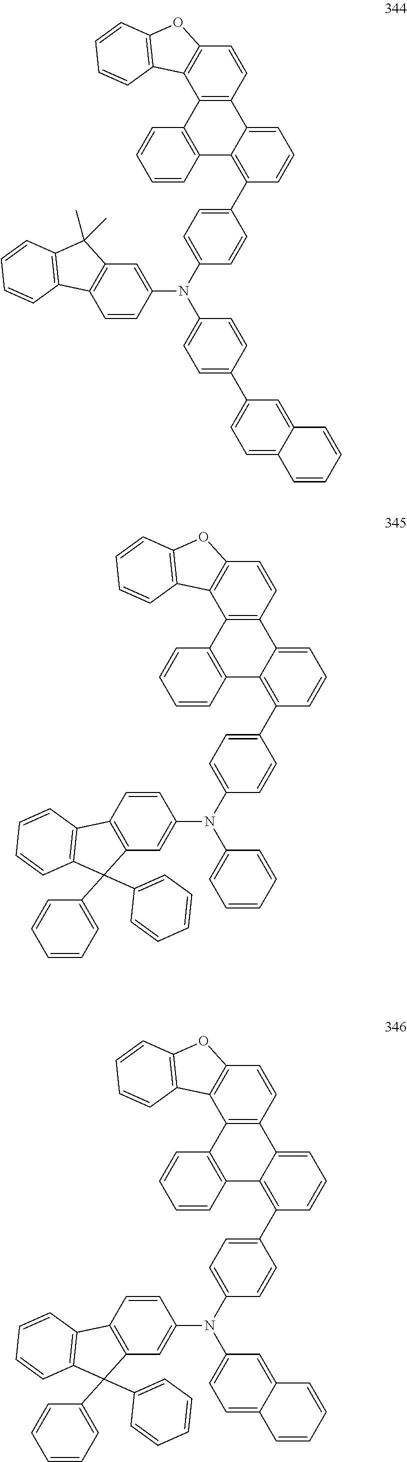 [SMILES: CC1(C)c2ccccc2-c2ccc(N(c3ccc(-c4ccc5ccccc5c4)cc3)c3ccc(-c4cccc5c6ccc7oc8ccccc8c7c6c6ccccc6c45)cc3)cc21 ; c1ccc(N(c2ccc(-c3cccc4c5ccc6oc7ccccc7c6c5c5ccccc5c34)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cccc6c7ccc8oc9ccccc9c8c7c7ccccc7c56)cc4)c4ccc5ccccc5c4)cc32)cc1]

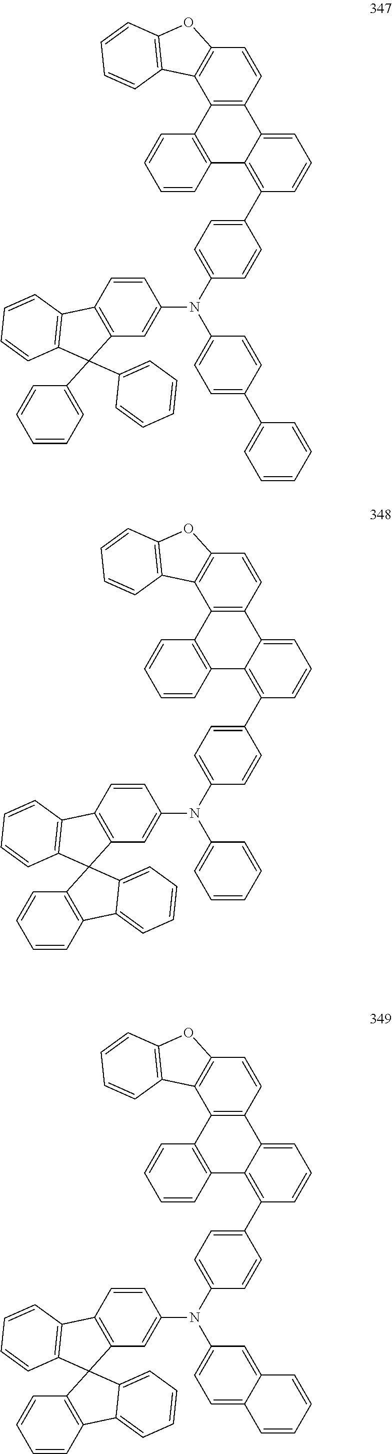 [SMILES: c1ccc(-c2ccc(N(c3ccc(-c4cccc5c6ccc7oc8ccccc8c7c6c6ccccc6c45)cc3)c3ccc4c(c3)C(c3ccccc3)(c3ccccc3)c3ccccc3-4)cc2)cc1 ; c1ccc(N(c2ccc(-c3cccc4c5ccc6oc7ccccc7c6c5c5ccccc5c34)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4cccc5c6ccc7oc8ccccc8c7c6c6ccccc6c45)cc3)c3ccc4ccccc4c3)cc21]

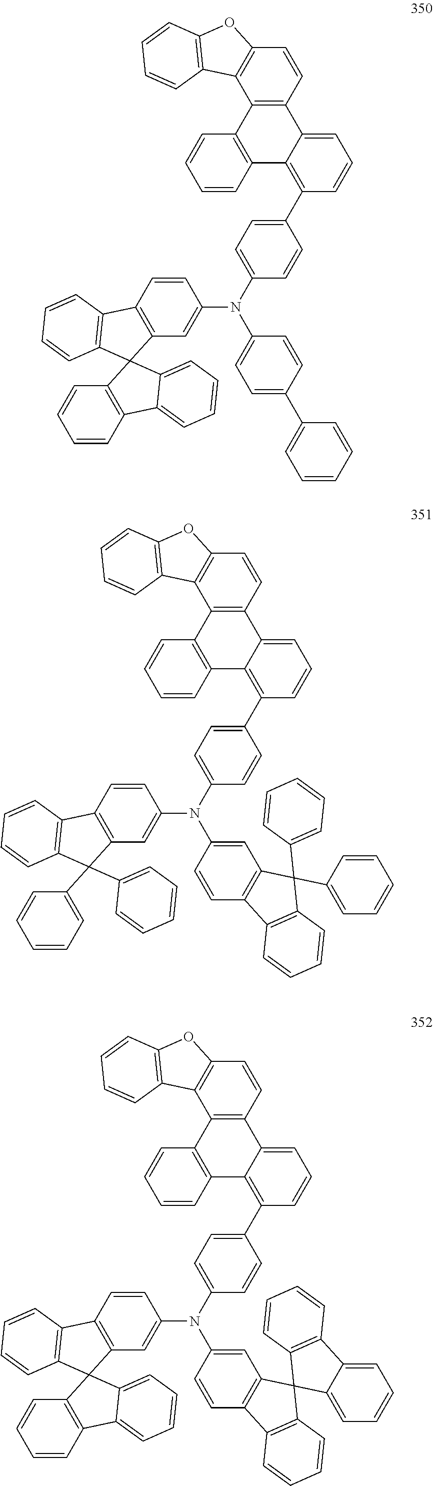 [SMILES: c1ccc(-c2ccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)cc2)cc1 ; c1ccc(C2(c3ccccc3)c3ccccc3-c3ccc(N(c4ccc(-c5cccc6c7ccc8oc9ccccc9c8c7c7ccccc7c56)cc4)c4ccc5c(c4)C(c4ccccc4)(c4ccccc4)c4ccccc4-5)cc32)cc1 ; c1ccc2c(c1)-c1ccccc1C21c2ccccc2-c2ccc(N(c3ccc(-c4cccc5c6ccc7oc8ccccc8c7c6c6ccccc6c45)cc3)c3ccc4c(c3)C3(c5ccccc5-c5ccccc53)c3ccccc3-4)cc21]

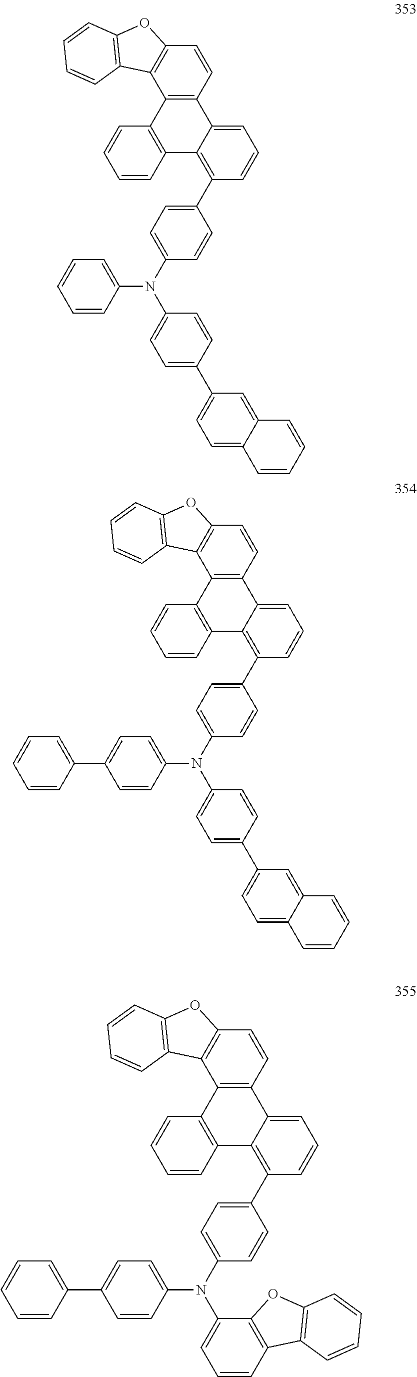 [SMILES: c1ccc(N(c2ccc(-c3ccc4ccccc4c3)cc2)c2ccc(-c3cccc4c5ccc6oc7ccccc7c6c5c5ccccc5c34)cc2)cc1 ; c1ccc(-c2ccc(N(c3ccc(-c4ccc5ccccc5c4)cc3)c3ccc(-c4cccc5c6ccc7oc8ccccc8c7c6c6ccccc6c45)cc3)cc2)cc1 ; c1ccc(-c2ccc(N(c3ccc(-c4cccc5c6ccc7oc8ccccc8c7c6c6ccccc6c45)cc3)c3cccc4c3oc3ccccc34)cc2)cc1]

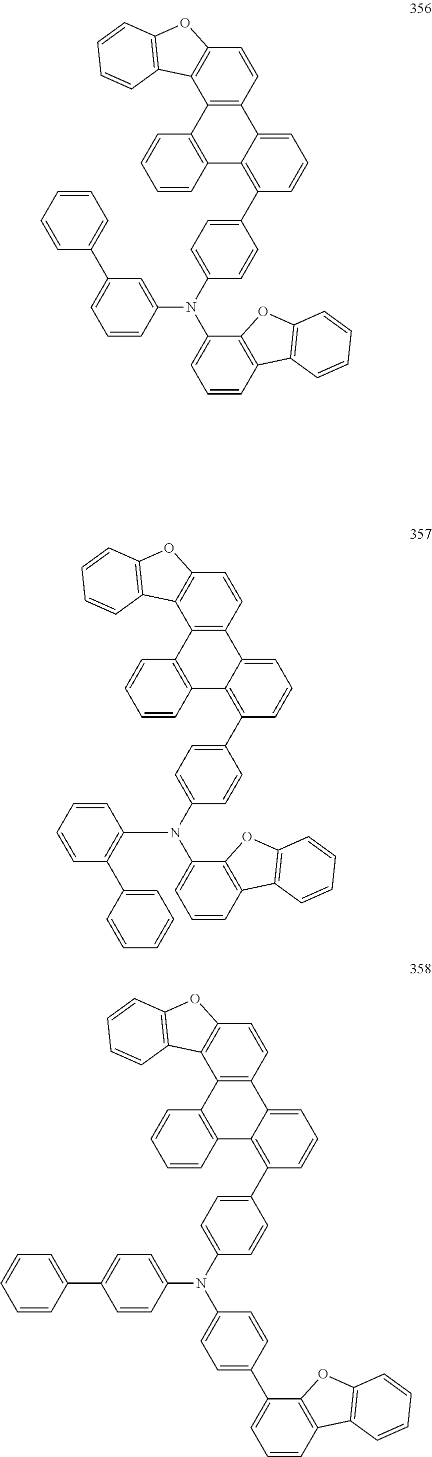 [SMILES: c1ccc(-c2cccc(N(c3ccc(-c4cccc5c6ccc7oc8ccccc8c7c6c6ccccc6c45)cc3)c3cccc4c3oc3ccccc34)c2)cc1 ; c1ccc(-c2ccccc2N(c2ccc(-c3cccc4c5ccc6oc7ccccc7c6c5c5ccccc5c34)cc2)c2cccc3c2oc2ccccc23)cc1 ; c1ccc(-c2ccc(N(c3ccc(-c4cccc5c4oc4ccccc45)cc3)c3ccc(-c4cccc5c6ccc7oc8ccccc8c7c6c6ccccc6c45)cc3)cc2)cc1]

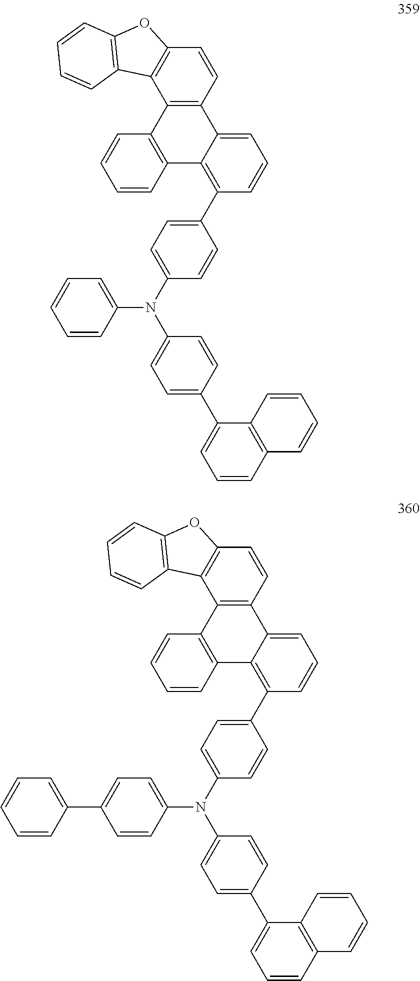 [SMILES: c1ccc(N(c2ccc(-c3cccc4ccccc34)cc2)c2ccc(-c3cccc4c5ccc6oc7ccccc7c6c5c5ccccc5c34)cc2)cc1 ; c1ccc(-c2ccc(N(c3ccc(-c4cccc5ccccc45)cc3)c3ccc(-c4cccc5c6ccc7oc8ccccc8c7c6c6ccccc6c45)cc3)cc2)cc1]

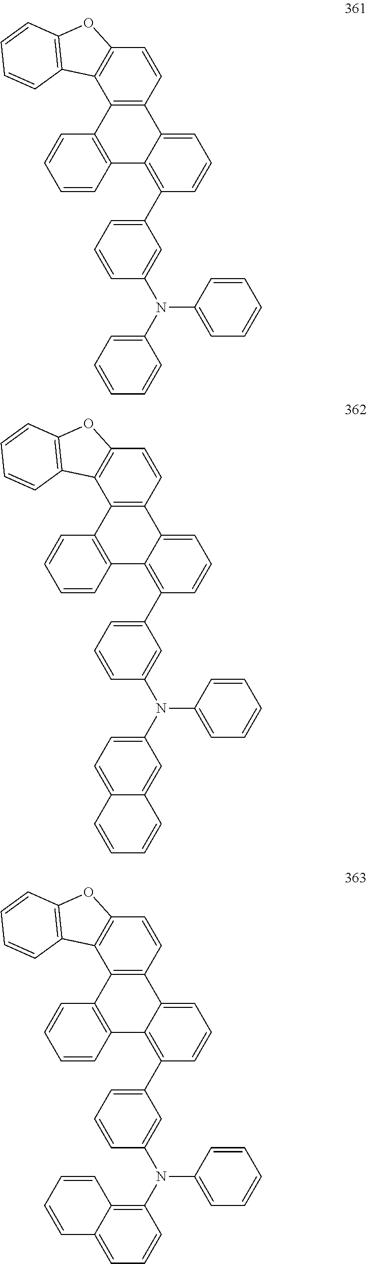

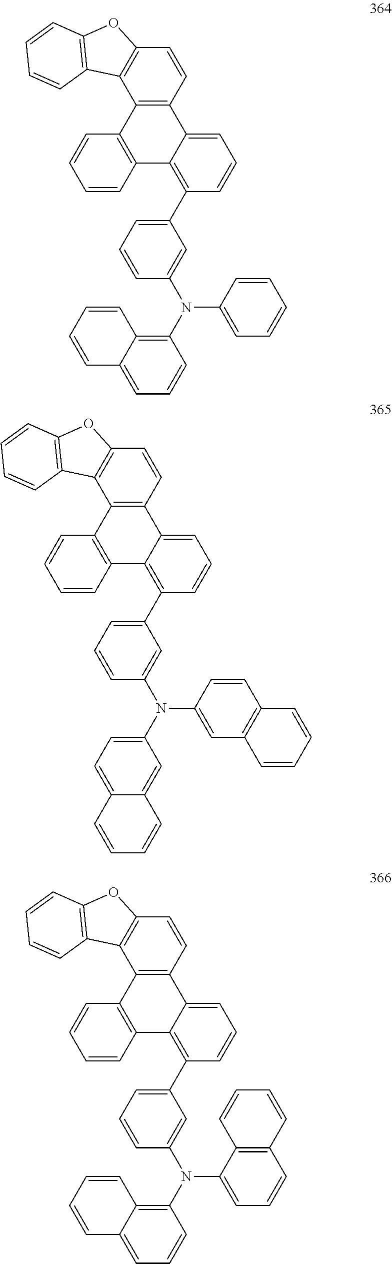

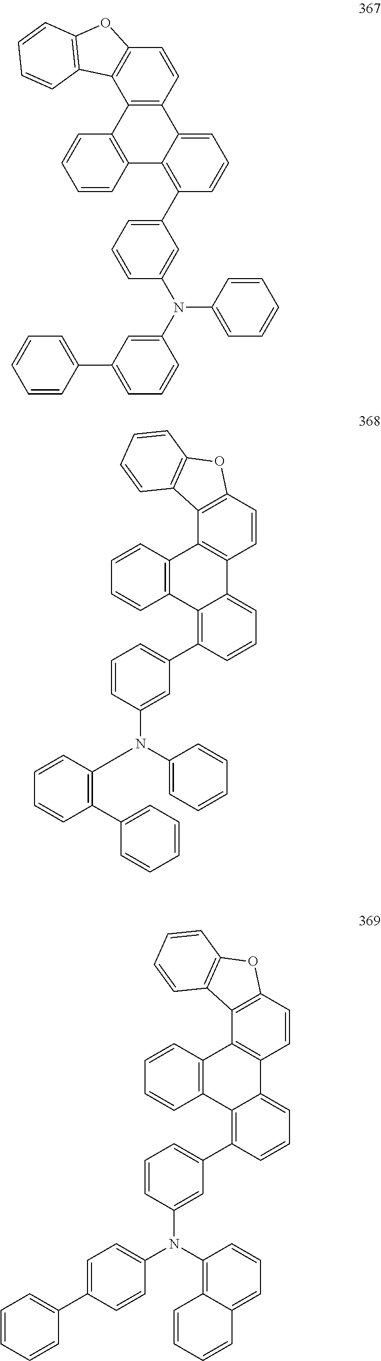

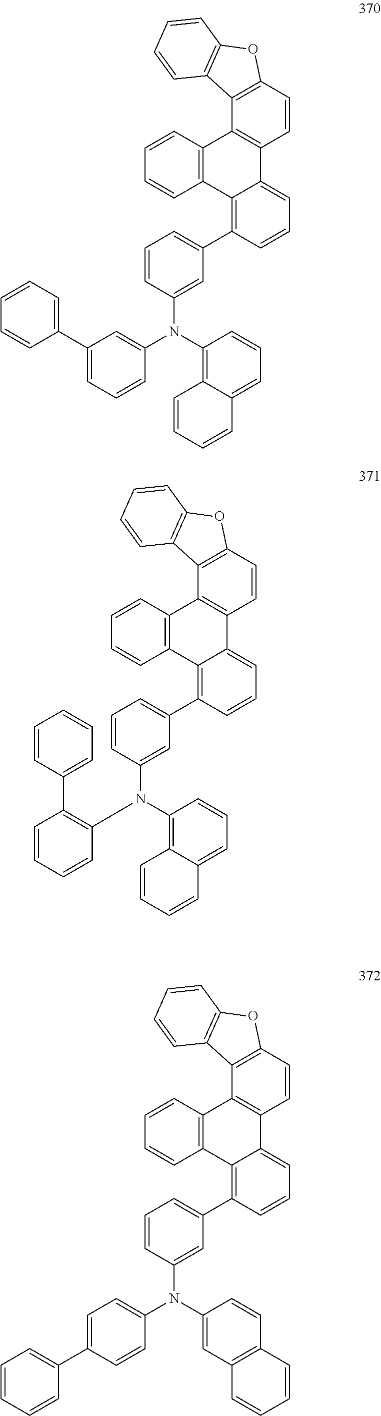 [SMILES: c1ccc(-c2cccc(N(c3cccc(-c4cccc5c6ccc7oc8ccccc8c7c6c6ccccc6c45)c3)c3cccc4ccccc34)c2)cc1 ; c1ccc(-c2ccccc2N(c2cccc(-c3cccc4c5ccc6oc7ccccc7c6c5c5ccccc5c34)c2)c2cccc3ccccc23)cc1 ; c1ccc(-c2ccc(N(c3cccc(-c4cccc5c6ccc7oc8ccccc8c7c6c6ccccc6c45)c3)c3ccc4ccccc4c3)cc2)cc1]

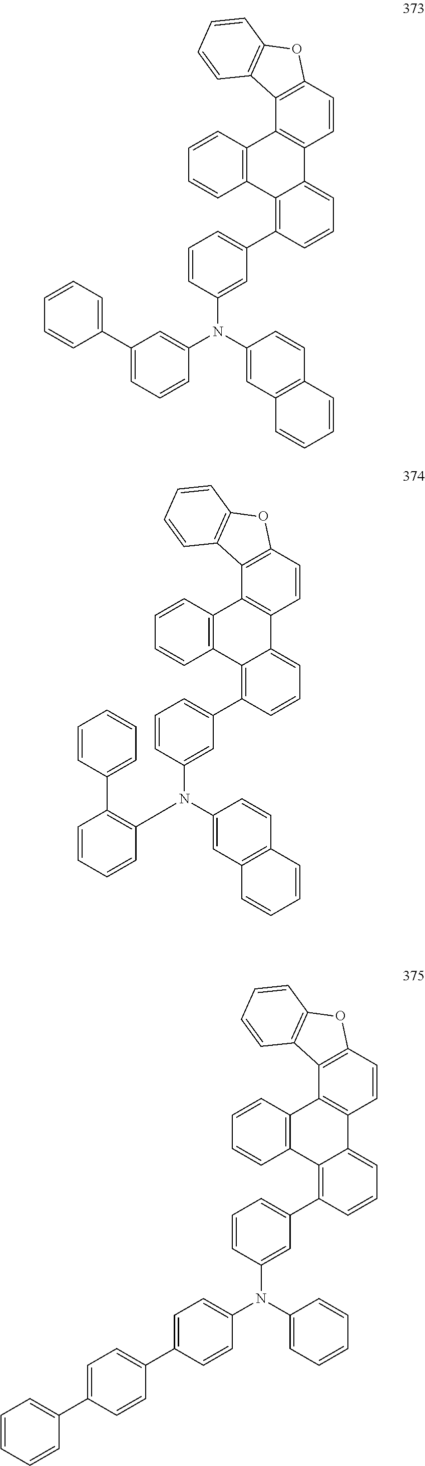 [SMILES: c1ccc(-c2cccc(N(c3cccc(-c4cccc5c6ccc7oc8ccccc8c7c6c6ccccc6c45)c3)c3ccc4ccccc4c3)c2)cc1 ; c1ccc(-c2ccccc2N(c2cccc(-c3cccc4c5ccc6oc7ccccc7c6c5c5ccccc5c34)c2)c2ccc3ccccc3c2)cc1 ; c1ccc(-c2ccc(-c3ccc(N(c4ccccc4)c4cccc(-c5cccc6c7ccc8oc9ccccc9c8c7c7ccccc7c56)c4)cc3)cc2)cc1]

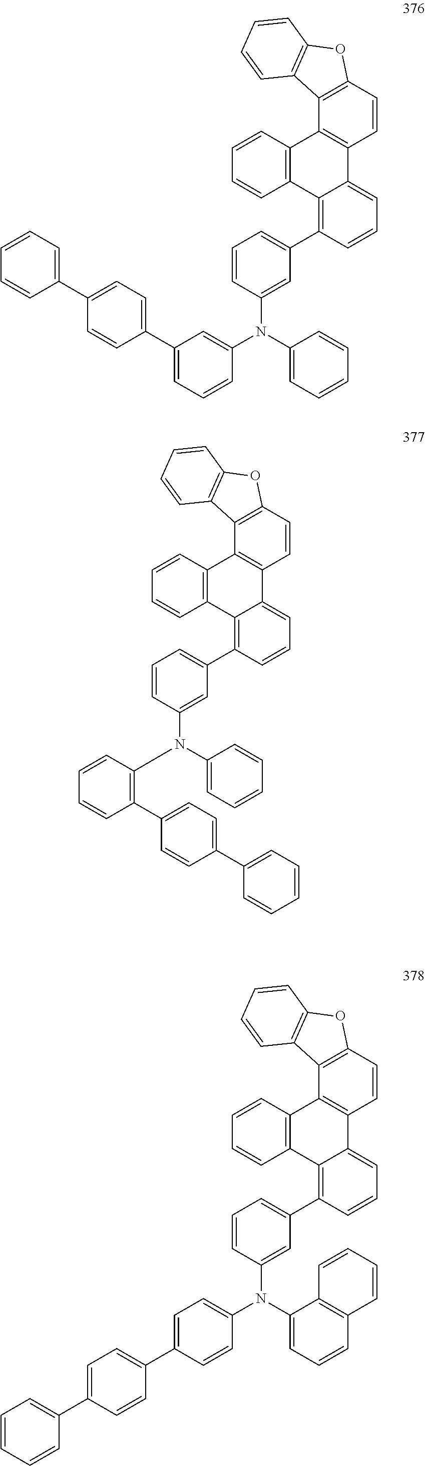 [SMILES: c1ccc(-c2ccc(-c3cccc(N(c4ccccc4)c4cccc(-c5cccc6c7ccc8oc9ccccc9c8c7c7ccccc7c56)c4)c3)cc2)cc1 ; c1ccc(-c2ccc(-c3ccccc3N(c3ccccc3)c3cccc(-c4cccc5c6ccc7oc8ccccc8c7c6c6ccccc6c45)c3)cc2)cc1 ; c1ccc(-c2ccc(-c3ccc(N(c4cccc(-c5cccc6c7ccc8oc9ccccc9c8c7c7ccccc7c56)c4)c4cccc5ccccc45)cc3)cc2)cc1]

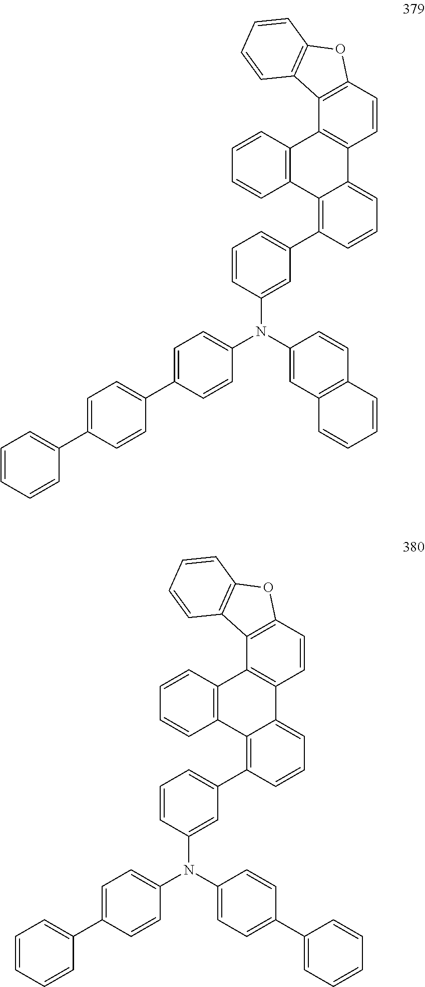 [SMILES: c1ccc(-c2ccc(-c3ccc(N(c4cccc(-c5cccc6c7ccc8oc9ccccc9c8c7c7ccccc7c56)c4)c4ccc5ccccc5c4)cc3)cc2)cc1 ; c1ccc(-c2ccc(N(c3ccc(-c4ccccc4)cc3)c3cccc(-c4cccc5c6ccc7oc8ccccc8c7c6c6ccccc6c45)c3)cc2)cc1]

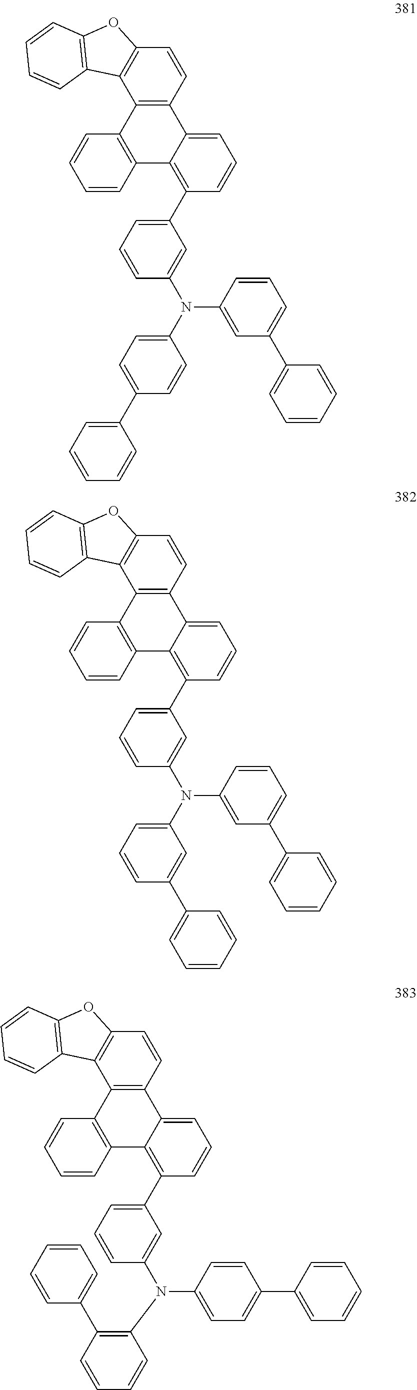

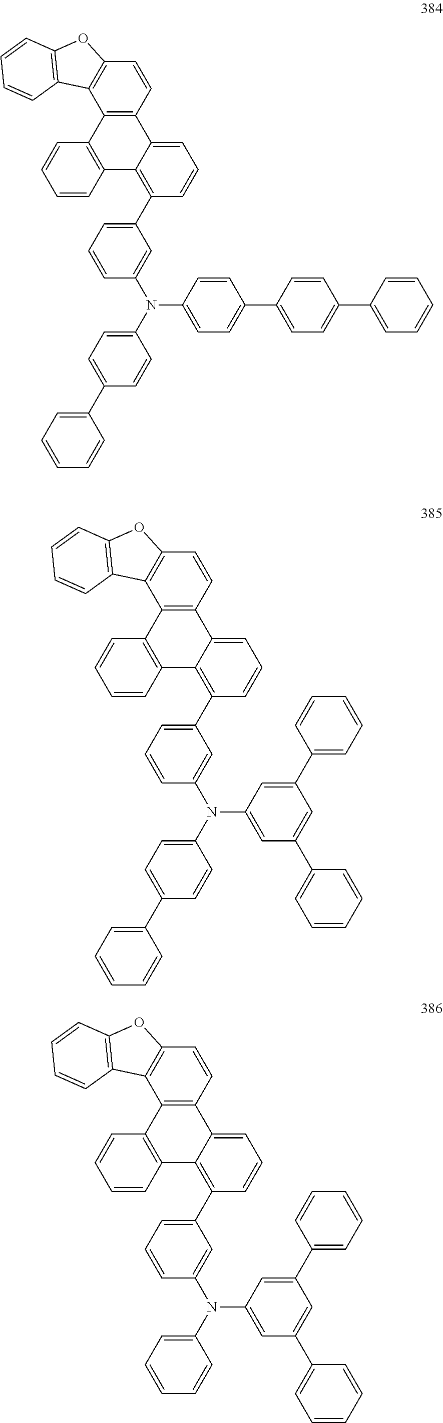

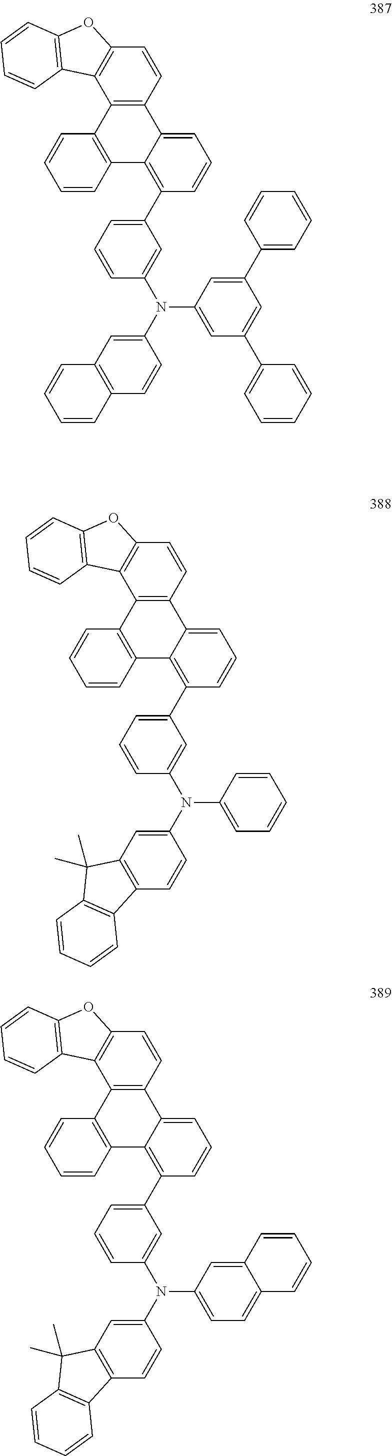

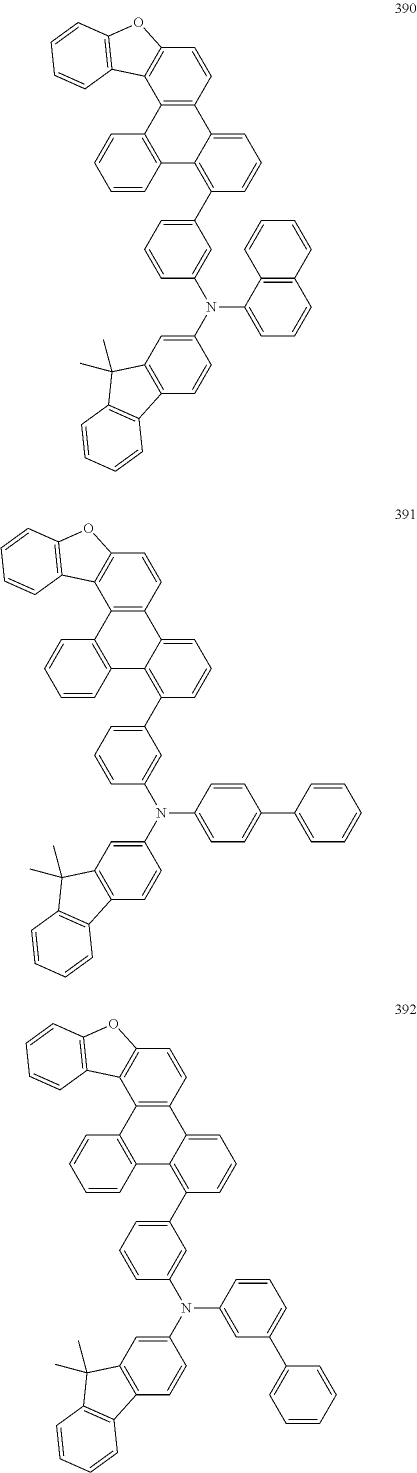 [SMILES: CC1(C)c2ccccc2-c2ccc(N(c3cccc(-c4cccc5c6ccc7oc8ccccc8c7c6c6ccccc6c45)c3)c3cccc4ccccc34)cc21 ; CC1(C)c2ccccc2-c2ccc(N(c3ccc(-c4ccccc4)cc3)c3cccc(-c4cccc5c6ccc7oc8ccccc8c7c6c6ccccc6c45)c3)cc21 ; CC1(C)c2ccccc2-c2ccc(N(c3cccc(-c4ccccc4)c3)c3cccc(-c4cccc5c6ccc7oc8ccccc8c7c6c6ccccc6c45)c3)cc21]

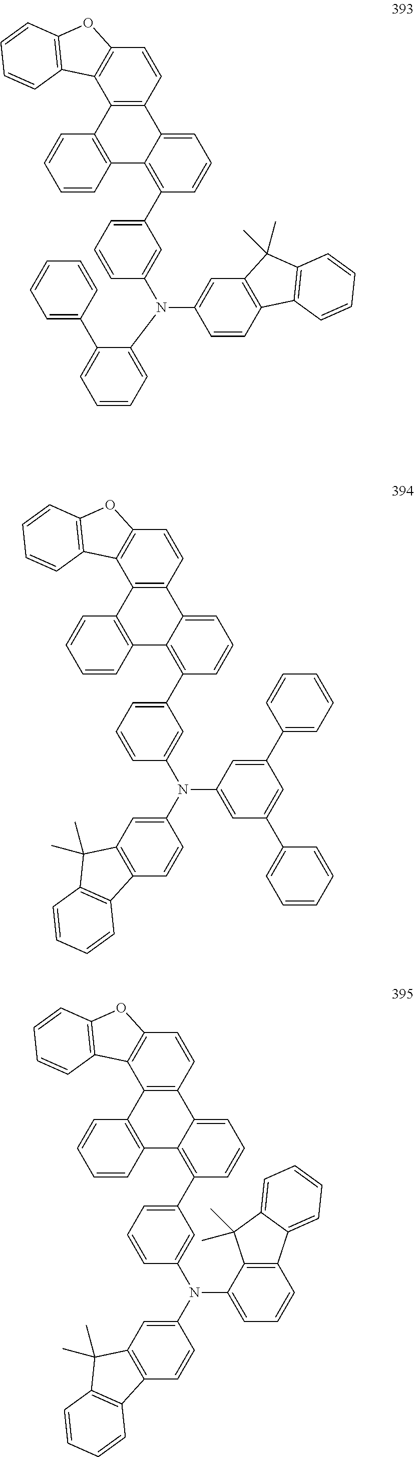 [SMILES: CC1(C)c2ccccc2-c2ccc(N(c3cccc(-c4cccc5c6ccc7oc8ccccc8c7c6c6ccccc6c45)c3)c3ccccc3-c3ccccc3)cc21 ; CC1(C)c2ccccc2-c2ccc(N(c3cc(-c4ccccc4)cc(-c4ccccc4)c3)c3cccc(-c4cccc5c6ccc7oc8ccccc8c7c6c6ccccc6c45)c3)cc21 ; CC1(C)c2ccccc2-c2ccc(N(c3cccc(-c4cccc5c6ccc7oc8ccccc8c7c6c6ccccc6c45)c3)c3cccc4c3C(C)(C)c3ccccc3-4)cc21]

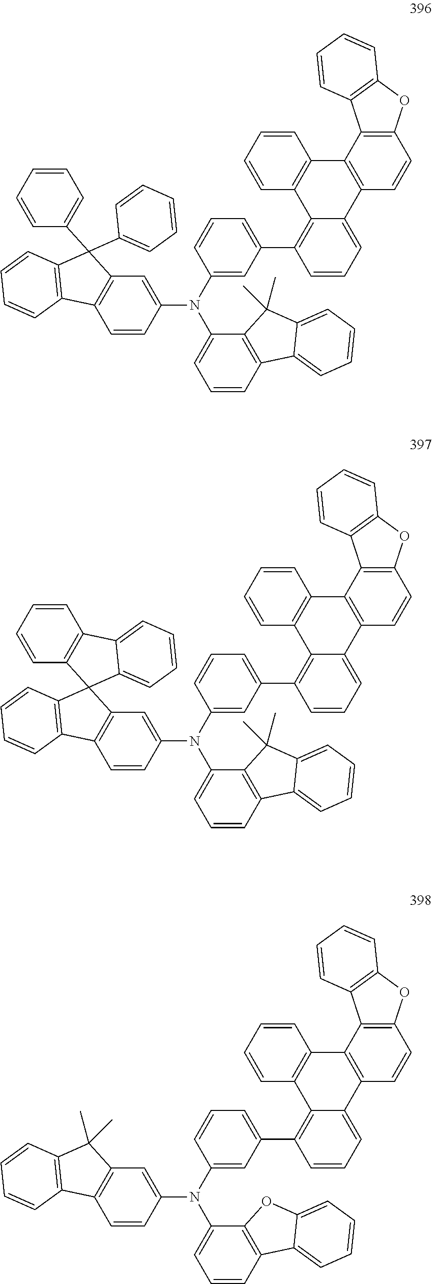 [SMILES: CC1(C)c2ccccc2-c2cccc(N(c3cccc(-c4cccc5c6ccc7oc8ccccc8c7c6c6ccccc6c45)c3)c3ccc4c(c3)C(c3ccccc3)(c3ccccc3)c3ccccc3-4)c21 ; CC1(C)c2ccccc2-c2cccc(N(c3cccc(-c4cccc5c6ccc7oc8ccccc8c7c6c6ccccc6c45)c3)c3ccc4c(c3)C3(c5ccccc5-c5ccccc53)c3ccccc3-4)c21 ; CC1(C)c2ccccc2-c2ccc(N(c3cccc(-c4cccc5c6ccc7oc8ccccc8c7c6c6ccccc6c45)c3)c3cccc4c3oc3ccccc34)cc21]

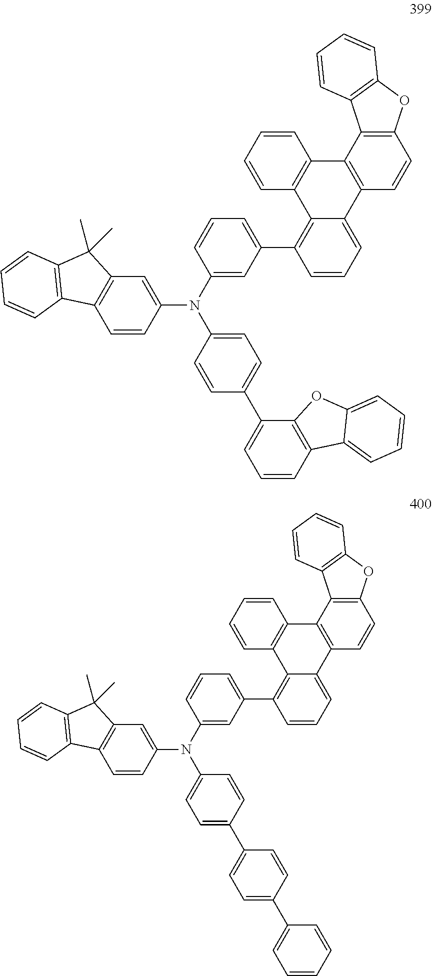

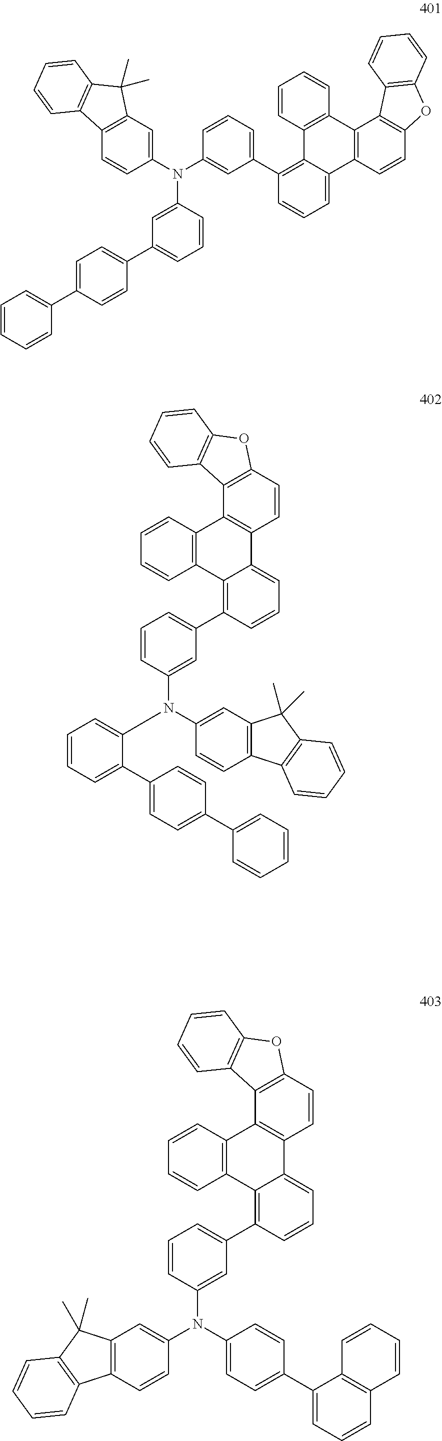 [SMILES: CC1(C)c2ccccc2-c2ccc(N(c3cccc(-c4ccc(-c5ccccc5)cc4)c3)c3cccc(-c4cccc5c6ccc7oc8ccccc8c7c6c6ccccc6c45)c3)cc21 ; CC1(C)c2ccccc2-c2ccc(N(c3cccc(-c4cccc5c6ccc7oc8ccccc8c7c6c6ccccc6c45)c3)c3ccccc3-c3ccc(-c4ccccc4)cc3)cc21 ; CC1(C)c2ccccc2-c2ccc(N(c3ccc(-c4cccc5ccccc45)cc3)c3cccc(-c4cccc5c6ccc7oc8ccccc8c7c6c6ccccc6c45)c3)cc21]

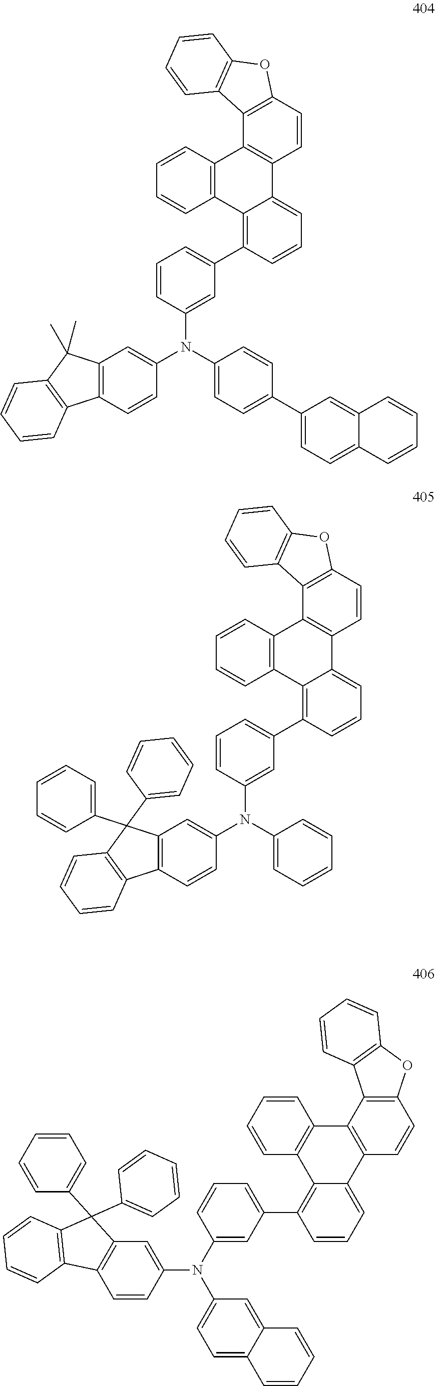 [SMILES: CC1(C)c2ccccc2-c2ccc(N(c3ccc(-c4ccc5ccccc5c4)cc3)c3cccc(-c4cccc5c6ccc7oc8ccccc8c7c6c6ccccc6c45)c3)cc21 ; c1ccc(N(c2cccc(-c3cccc4c5ccc6oc7ccccc7c6c5c5ccccc5c34)c2)c2ccc3c(c2)C(c2ccccc2)(c2ccccc2)c2ccccc2-3)cc1 ; c1ccc(C2(c3ccccc3)c3ccccc3-c3ccc(N(c4cccc(-c5cccc6c7ccc8oc9ccccc9c8c7c7ccccc7c56)c4)c4ccc5ccccc5c4)cc32)cc1]

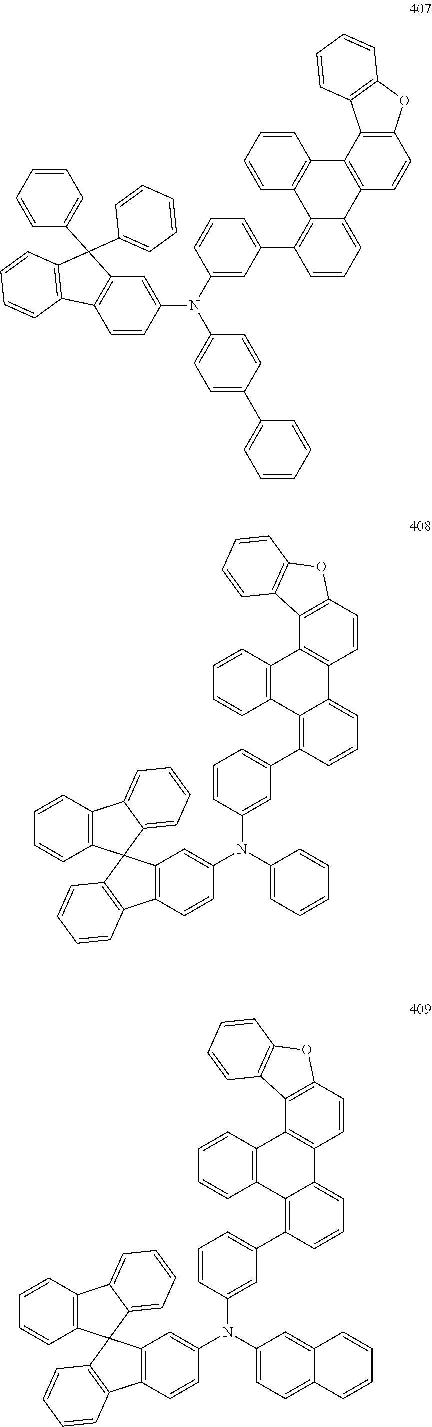 [SMILES: c1ccc(-c2ccc(N(c3cccc(-c4cccc5c6ccc7oc8ccccc8c7c6c6ccccc6c45)c3)c3ccc4c(c3)C(c3ccccc3)(c3ccccc3)c3ccccc3-4)cc2)cc1 ; c1ccc(N(c2cccc(-c3cccc4c5ccc6oc7ccccc7c6c5c5ccccc5c34)c2)c2ccc3c(c2)C2(c4ccccc4-c4ccccc42)c2ccccc2-3)cc1 ; c1cc(-c2cccc3c4ccc5oc6ccccc6c5c4c4ccccc4c23)cc(N(c2ccc3c(c2)C2(c4ccccc4-c4ccccc42)c2ccccc2-3)c2ccc3ccccc3c2)c1]

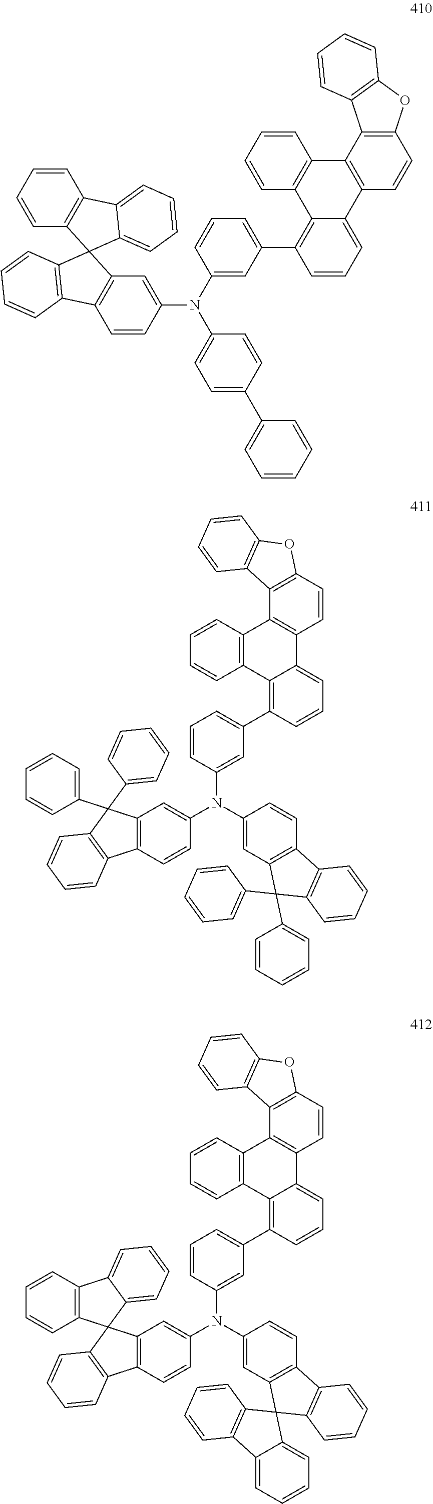

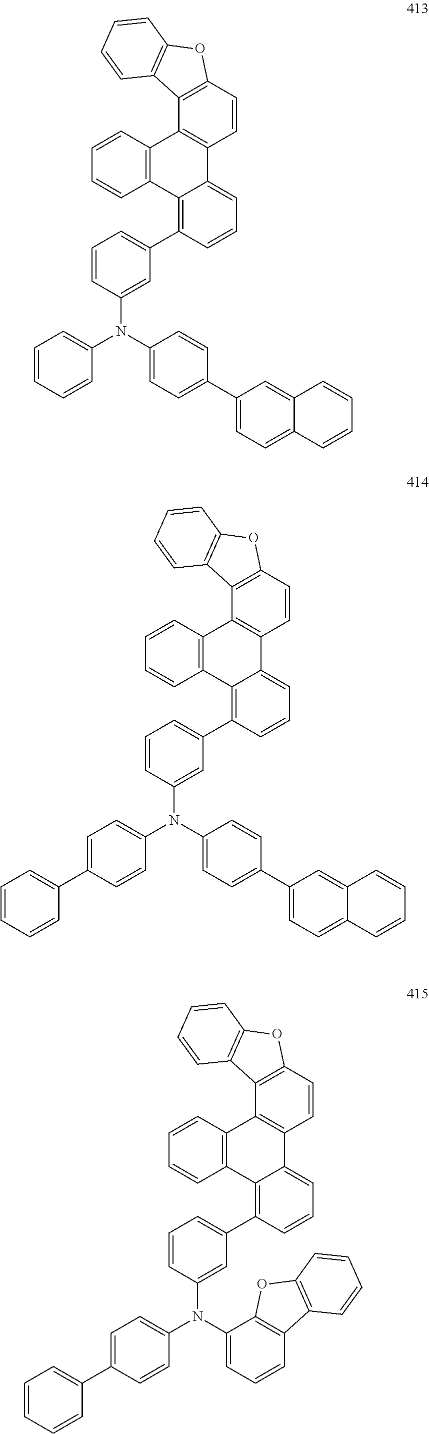 [SMILES: c1ccc(N(c2ccc(-c3ccc4ccccc4c3)cc2)c2cccc(-c3cccc4c5ccc6oc7ccccc7c6c5c5ccccc5c34)c2)cc1 ; c1ccc(-c2ccc(N(c3ccc(-c4ccc5ccccc5c4)cc3)c3cccc(-c4cccc5c6ccc7oc8ccccc8c7c6c6ccccc6c45)c3)cc2)cc1 ; c1ccc(-c2ccc(N(c3cccc(-c4cccc5c6ccc7oc8ccccc8c7c6c6ccccc6c45)c3)c3cccc4c3oc3ccccc34)cc2)cc1]

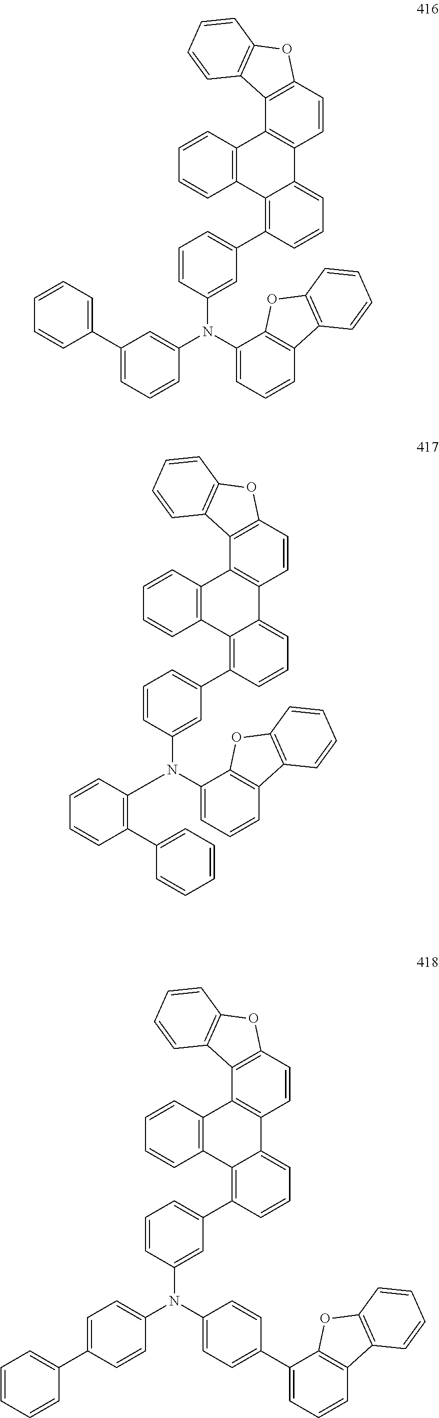 [SMILES: c1ccc(-c2cccc(N(c3cccc(-c4cccc5c6ccc7oc8ccccc8c7c6c6ccccc6c45)c3)c3cccc4c3oc3ccccc34)c2)cc1 ; c1ccc(-c2ccccc2N(c2cccc(-c3cccc4c5ccc6oc7ccccc7c6c5c5ccccc5c34)c2)c2cccc3c2oc2ccccc23)cc1 ; c1ccc(-c2ccc(N(c3ccc(-c4cccc5c4oc4ccccc45)cc3)c3cccc(-c4cccc5c6ccc7oc8ccccc8c7c6c6ccccc6c45)c3)cc2)cc1]

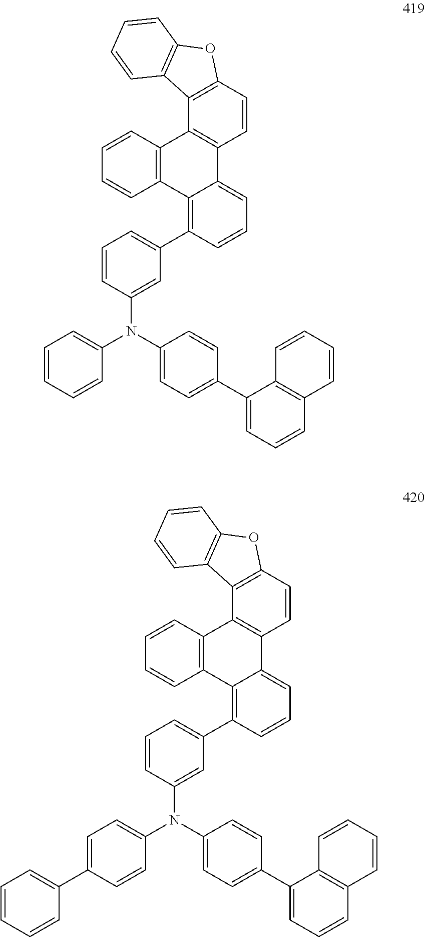 [SMILES: c1ccc(N(c2ccc(-c3cccc4ccccc34)cc2)c2cccc(-c3cccc4c5ccc6oc7ccccc7c6c5c5ccccc5c34)c2)cc1 ; c1ccc(-c2ccc(N(c3ccc(-c4cccc5ccccc45)cc3)c3cccc(-c4cccc5c6ccc7oc8ccccc8c7c6c6ccccc6c45)c3)cc2)cc1]

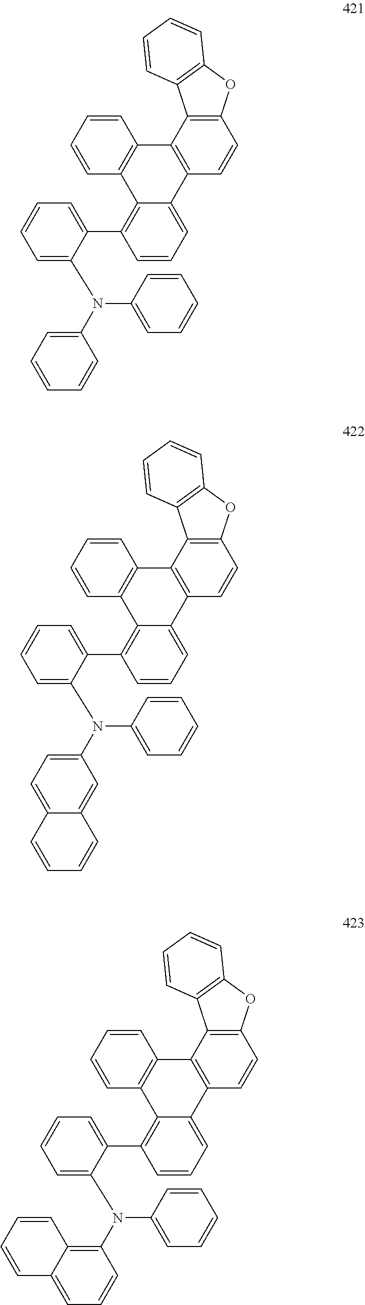 [SMILES: c1ccc(N(c2ccccc2)c2ccccc2-c2cccc3c4ccc5oc6ccccc6c5c4c4ccccc4c23)cc1 ; c1ccc(N(c2ccc3ccccc3c2)c2ccccc2-c2cccc3c4ccc5oc6ccccc6c5c4c4ccccc4c23)cc1 ; c1ccc(N(c2ccccc2-c2cccc3c4ccc5oc6ccccc6c5c4c4ccccc4c23)c2cccc3ccccc23)cc1]

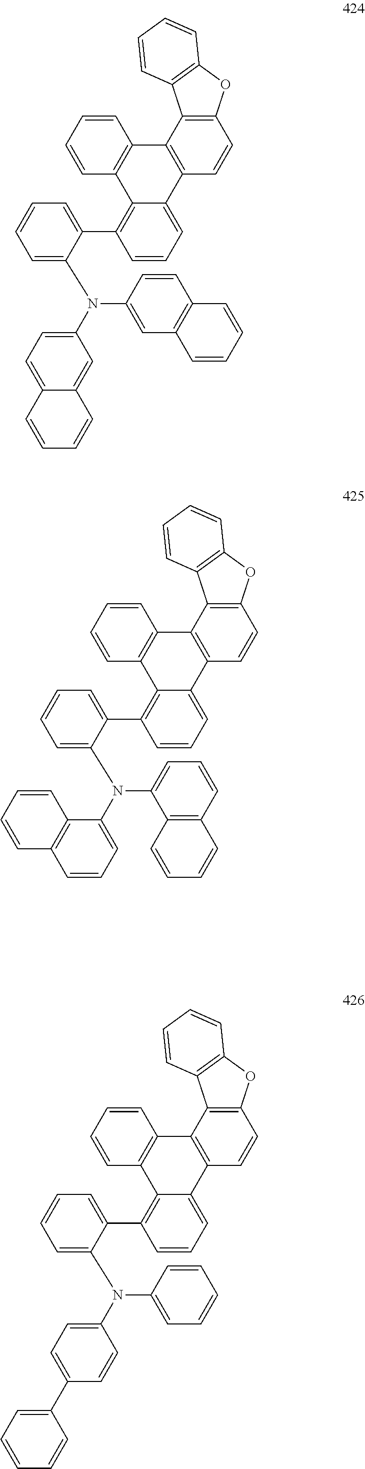 [SMILES: c1ccc(N(c2ccc3ccccc3c2)c2ccc3ccccc3c2)c(-c2cccc3c4ccc5oc6ccccc6c5c4c4ccccc4c23)c1 ; c1ccc(N(c2cccc3ccccc23)c2cccc3ccccc23)c(-c2cccc3c4ccc5oc6ccccc6c5c4c4ccccc4c23)c1 ; c1ccc(-c2ccc(N(c3ccccc3)c3ccccc3-c3cccc4c5ccc6oc7ccccc7c6c5c5ccccc5c34)cc2)cc1]

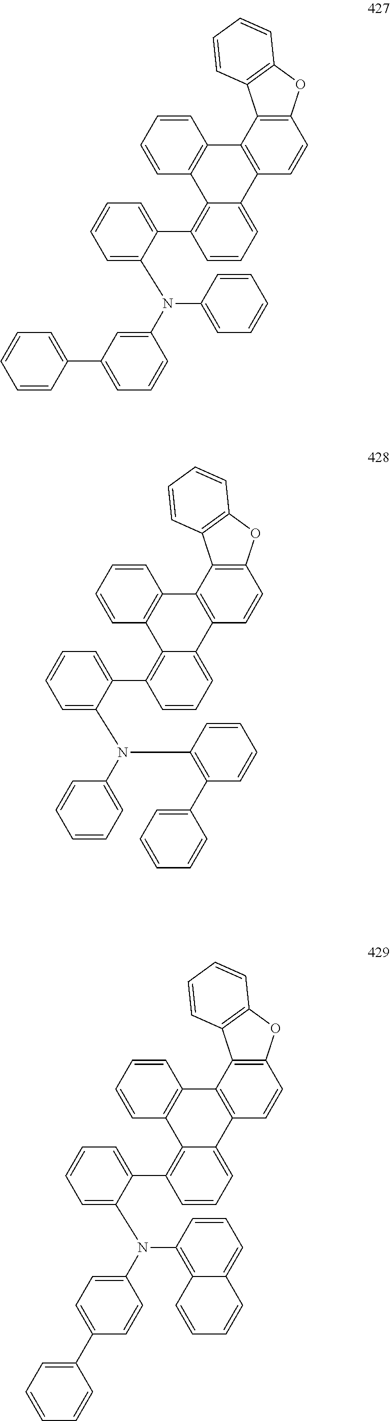 [SMILES: c1ccc(-c2cccc(N(c3ccccc3)c3ccccc3-c3cccc4c5ccc6oc7ccccc7c6c5c5ccccc5c34)c2)cc1 ; c1ccc(-c2ccccc2N(c2ccccc2)c2ccccc2-c2cccc3c4ccc5oc6ccccc6c5c4c4ccccc4c23)cc1 ; c1ccc(-c2ccc(N(c3ccccc3-c3cccc4c5ccc6oc7ccccc7c6c5c5ccccc5c34)c3cccc4ccccc34)cc2)cc1]

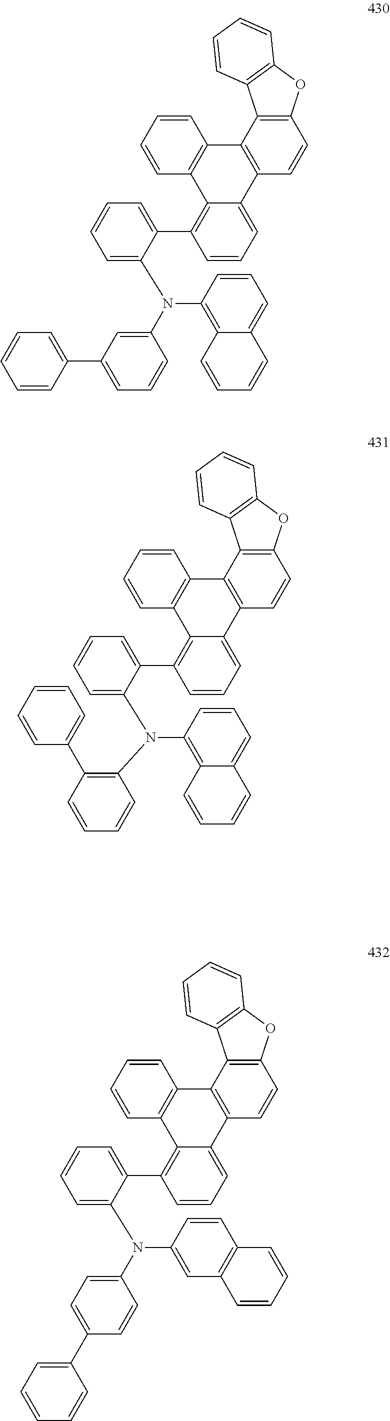 [SMILES: c1ccc(-c2cccc(N(c3ccccc3-c3cccc4c5ccc6oc7ccccc7c6c5c5ccccc5c34)c3cccc4ccccc34)c2)cc1 ; c1ccc(-c2ccccc2N(c2ccccc2-c2cccc3c4ccc5oc6ccccc6c5c4c4ccccc4c23)c2cccc3ccccc23)cc1 ; c1ccc(-c2ccc(N(c3ccc4ccccc4c3)c3ccccc3-c3cccc4c5ccc6oc7ccccc7c6c5c5ccccc5c34)cc2)cc1]

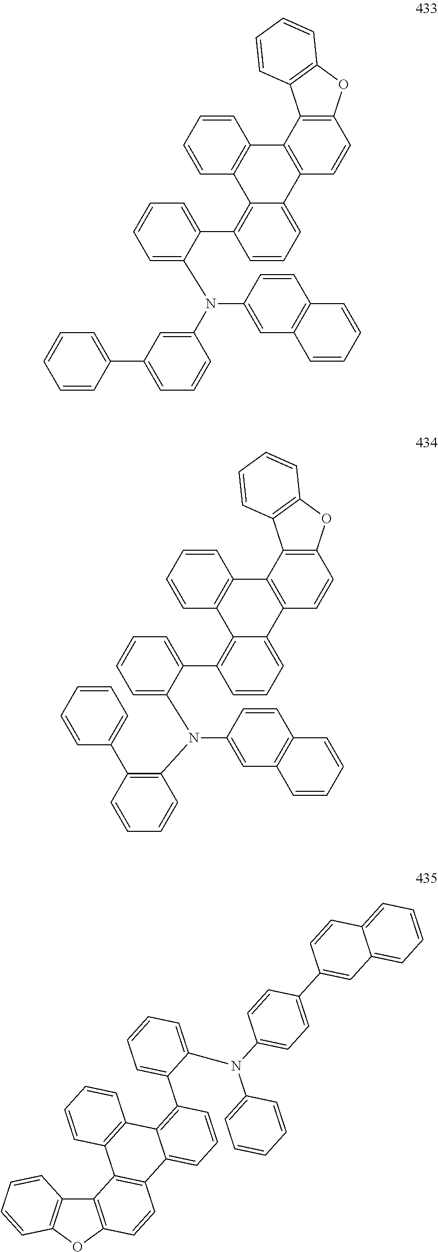 [SMILES: c1ccc(-c2cccc(N(c3ccc4ccccc4c3)c3ccccc3-c3cccc4c5ccc6oc7ccccc7c6c5c5ccccc5c34)c2)cc1 ; c1ccc(-c2ccccc2N(c2ccc3ccccc3c2)c2ccccc2-c2cccc3c4ccc5oc6ccccc6c5c4c4ccccc4c23)cc1 ; c1ccc(N(c2ccc(-c3ccc4ccccc4c3)cc2)c2ccccc2-c2cccc3c4ccc5oc6ccccc6c5c4c4ccccc4c23)cc1]

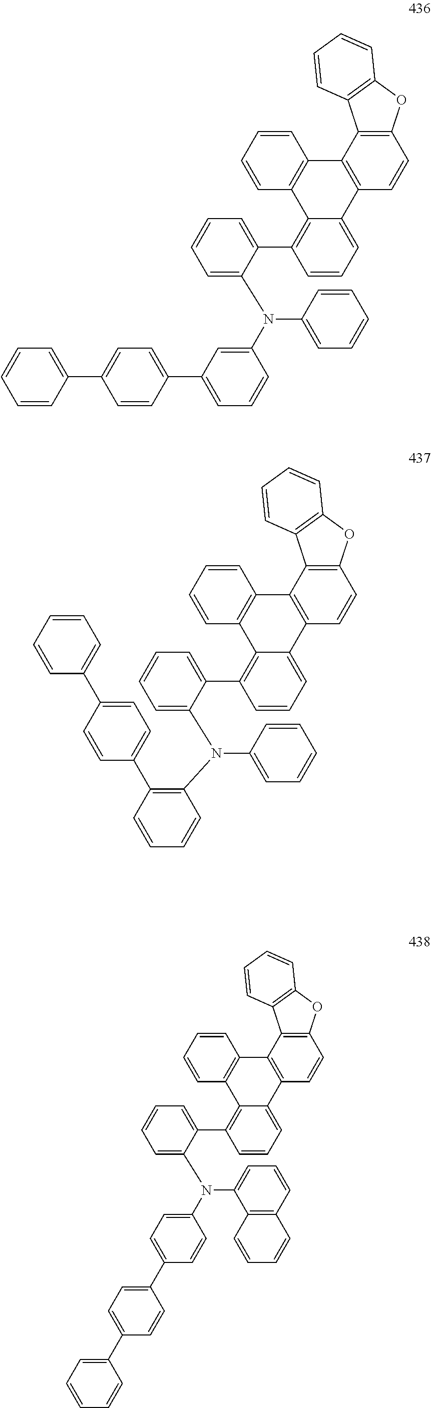 [SMILES: c1ccc(-c2ccc(-c3cccc(N(c4ccccc4)c4ccccc4-c4cccc5c6ccc7oc8ccccc8c7c6c6ccccc6c45)c3)cc2)cc1 ; c1ccc(-c2ccc(-c3ccccc3N(c3ccccc3)c3ccccc3-c3cccc4c5ccc6oc7ccccc7c6c5c5ccccc5c34)cc2)cc1 ; c1ccc(-c2ccc(-c3ccc(N(c4ccccc4-c4cccc5c6ccc7oc8ccccc8c7c6c6ccccc6c45)c4cccc5ccccc45)cc3)cc2)cc1]

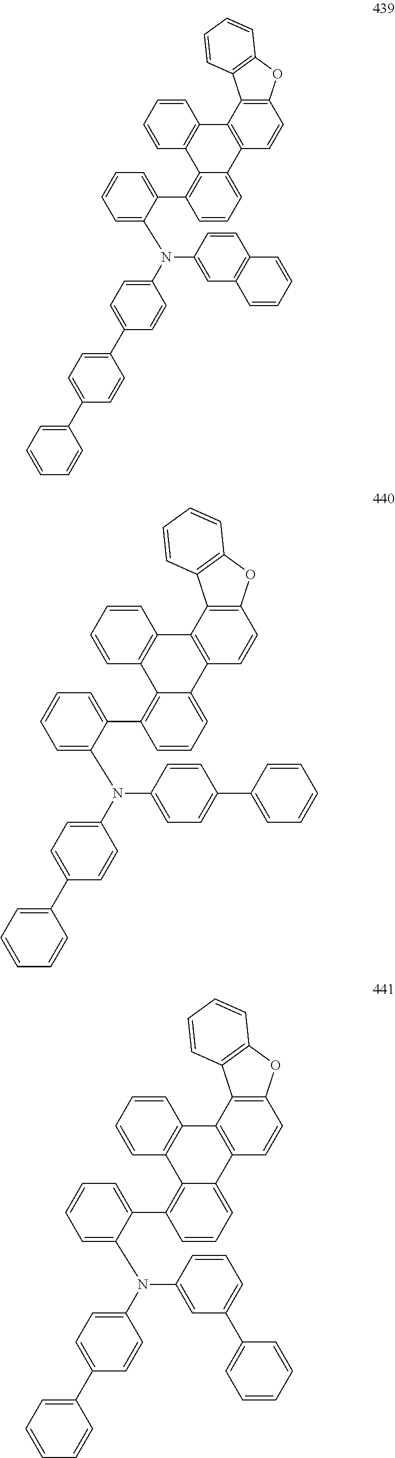 [SMILES: c1ccc(-c2ccc(-c3ccc(N(c4ccc5ccccc5c4)c4ccccc4-c4cccc5c6ccc7oc8ccccc8c7c6c6ccccc6c45)cc3)cc2)cc1 ; c1ccc(-c2ccc(N(c3ccc(-c4ccccc4)cc3)c3ccccc3-c3cccc4c5ccc6oc7ccccc7c6c5c5ccccc5c34)cc2)cc1 ; c1ccc(-c2ccc(N(c3cccc(-c4ccccc4)c3)c3ccccc3-c3cccc4c5ccc6oc7ccccc7c6c5c5ccccc5c34)cc2)cc1]

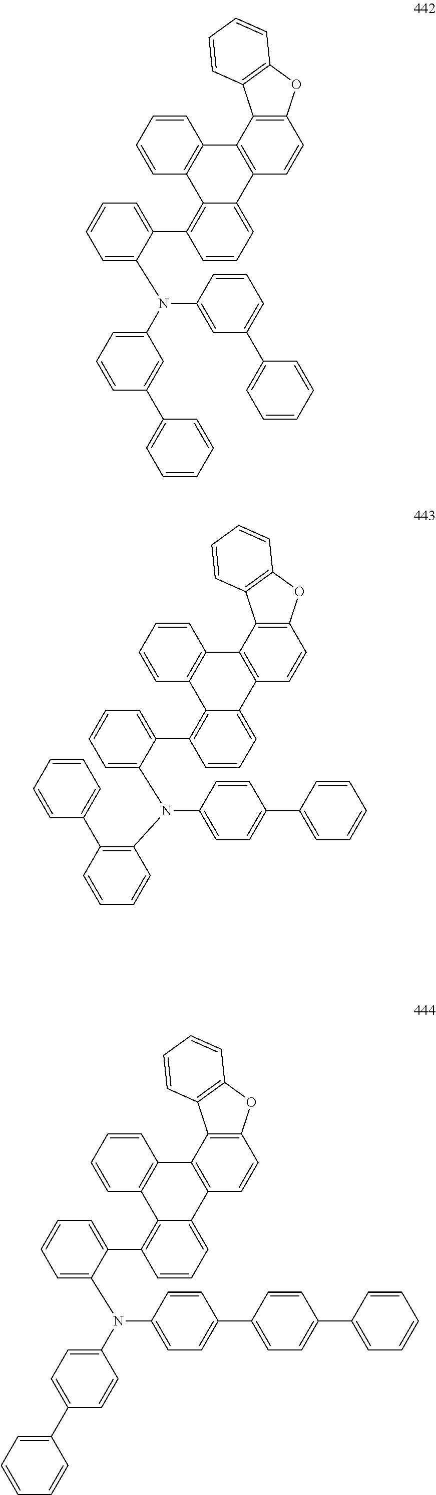 [SMILES: c1ccc(-c2cccc(N(c3cccc(-c4ccccc4)c3)c3ccccc3-c3cccc4c5ccc6oc7ccccc7c6c5c5ccccc5c34)c2)cc1 ; c1ccc(-c2ccc(N(c3ccccc3-c3ccccc3)c3ccccc3-c3cccc4c5ccc6oc7ccccc7c6c5c5ccccc5c34)cc2)cc1 ; c1ccc(-c2ccc(-c3ccc(N(c4ccc(-c5ccccc5)cc4)c4ccccc4-c4cccc5c6ccc7oc8ccccc8c7c6c6ccccc6c45)cc3)cc2)cc1]

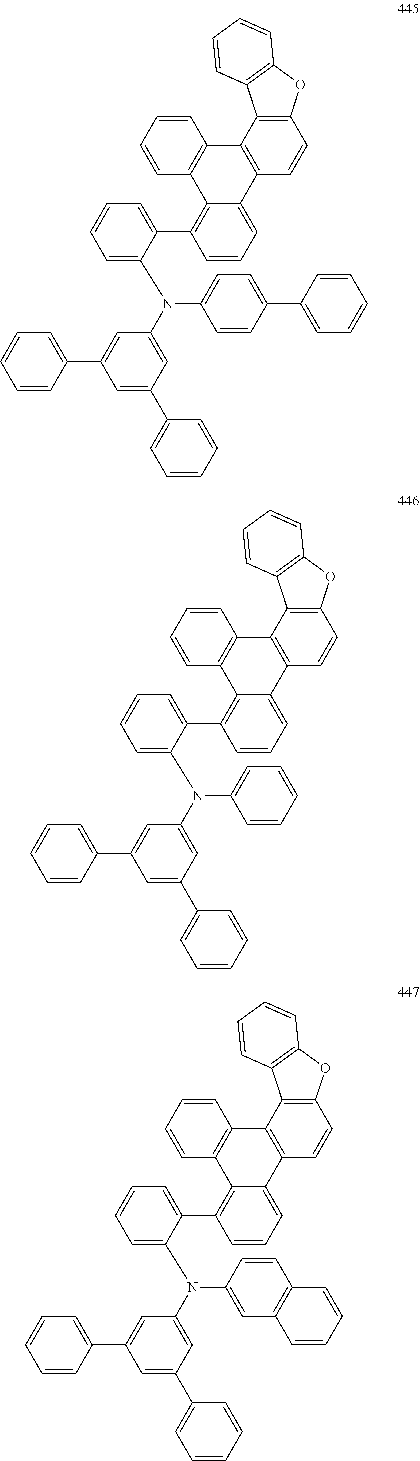 [SMILES: c1ccc(-c2ccc(N(c3cc(-c4ccccc4)cc(-c4ccccc4)c3)c3ccccc3-c3cccc4c5ccc6oc7ccccc7c6c5c5ccccc5c34)cc2)cc1 ; c1ccc(-c2cc(-c3ccccc3)cc(N(c3ccccc3)c3ccccc3-c3cccc4c5ccc6oc7ccccc7c6c5c5ccccc5c34)c2)cc1 ; c1ccc(-c2cc(-c3ccccc3)cc(N(c3ccc4ccccc4c3)c3ccccc3-c3cccc4c5ccc6oc7ccccc7c6c5c5ccccc5c34)c2)cc1]

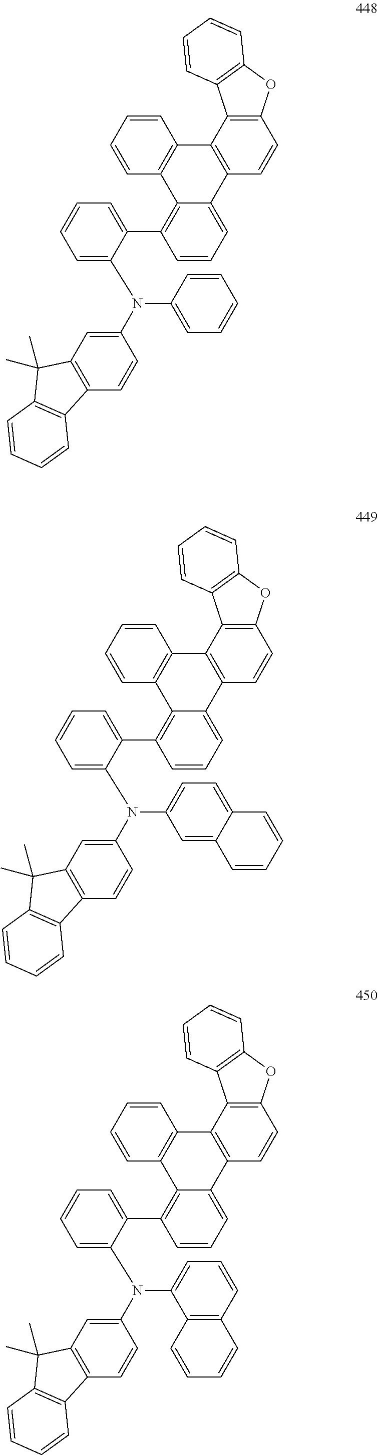 [SMILES: CC1(C)c2ccccc2-c2ccc(N(c3ccccc3)c3ccccc3-c3cccc4c5ccc6oc7ccccc7c6c5c5ccccc5c34)cc21 ; CC1(C)c2ccccc2-c2ccc(N(c3ccc4ccccc4c3)c3ccccc3-c3cccc4c5ccc6oc7ccccc7c6c5c5ccccc5c34)cc21 ; CC1(C)c2ccccc2-c2ccc(N(c3ccccc3-c3cccc4c5ccc6oc7ccccc7c6c5c5ccccc5c34)c3cccc4ccccc34)cc21]

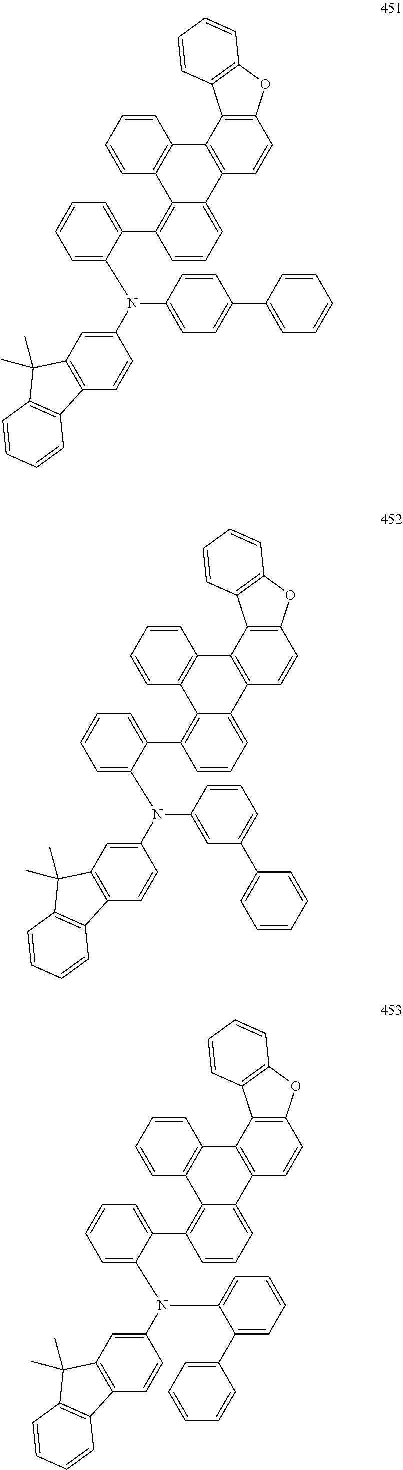 [SMILES: CC1(C)c2ccccc2-c2ccc(N(c3ccc(-c4ccccc4)cc3)c3ccccc3-c3cccc4c5ccc6oc7ccccc7c6c5c5ccccc5c34)cc21 ; CC1(C)c2ccccc2-c2ccc(N(c3cccc(-c4ccccc4)c3)c3ccccc3-c3cccc4c5ccc6oc7ccccc7c6c5c5ccccc5c34)cc21 ; CC1(C)c2ccccc2-c2ccc(N(c3ccccc3-c3ccccc3)c3ccccc3-c3cccc4c5ccc6oc7ccccc7c6c5c5ccccc5c34)cc21]

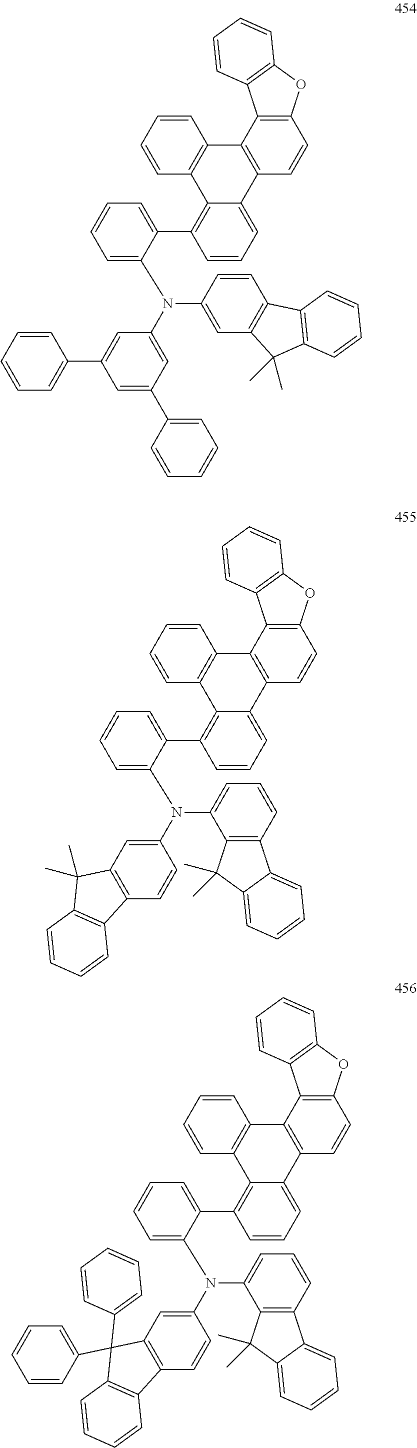 [SMILES: CC1(C)c2ccccc2-c2ccc(N(c3cc(-c4ccccc4)cc(-c4ccccc4)c3)c3ccccc3-c3cccc4c5ccc6oc7ccccc7c6c5c5ccccc5c34)cc21 ; CC1(C)c2ccccc2-c2ccc(N(c3ccccc3-c3cccc4c5ccc6oc7ccccc7c6c5c5ccccc5c34)c3cccc4c3C(C)(C)c3ccccc3-4)cc21 ; CC1(C)c2ccccc2-c2cccc(N(c3ccc4c(c3)C(c3ccccc3)(c3ccccc3)c3ccccc3-4)c3ccccc3-c3cccc4c5ccc6oc7ccccc7c6c5c5ccccc5c34)c21]

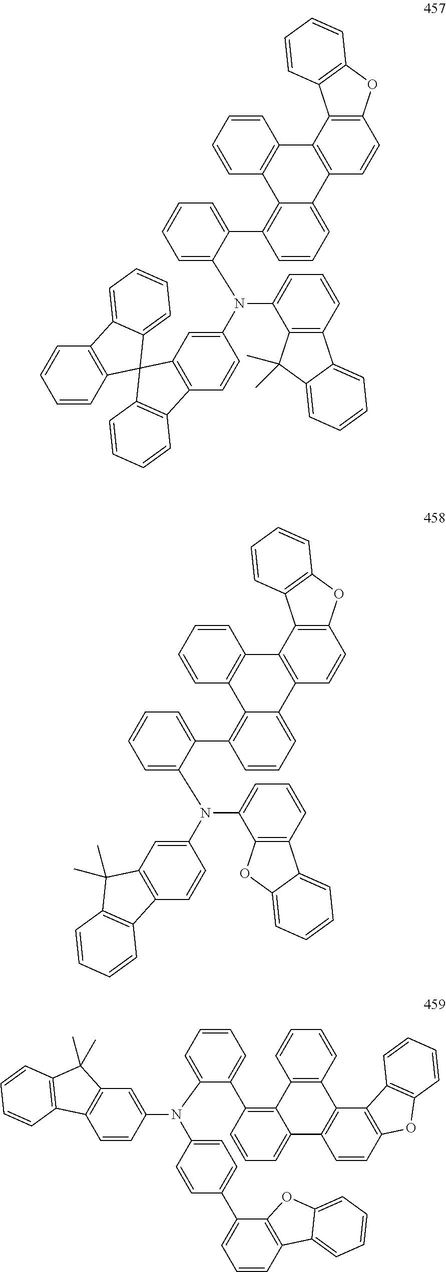 [SMILES: CC1(C)c2ccccc2-c2cccc(N(c3ccc4c(c3)C3(c5ccccc5-c5ccccc53)c3ccccc3-4)c3ccccc3-c3cccc4c5ccc6oc7ccccc7c6c5c5ccccc5c34)c21 ; CC1(C)c2ccccc2-c2ccc(N(c3ccccc3-c3cccc4c5ccc6oc7ccccc7c6c5c5ccccc5c34)c3cccc4c3oc3ccccc34)cc21 ; CC1(C)c2ccccc2-c2ccc(N(c3ccc(-c4cccc5c4oc4ccccc45)cc3)c3ccccc3-c3cccc4c5ccc6oc7ccccc7c6c5c5ccccc5c34)cc21]

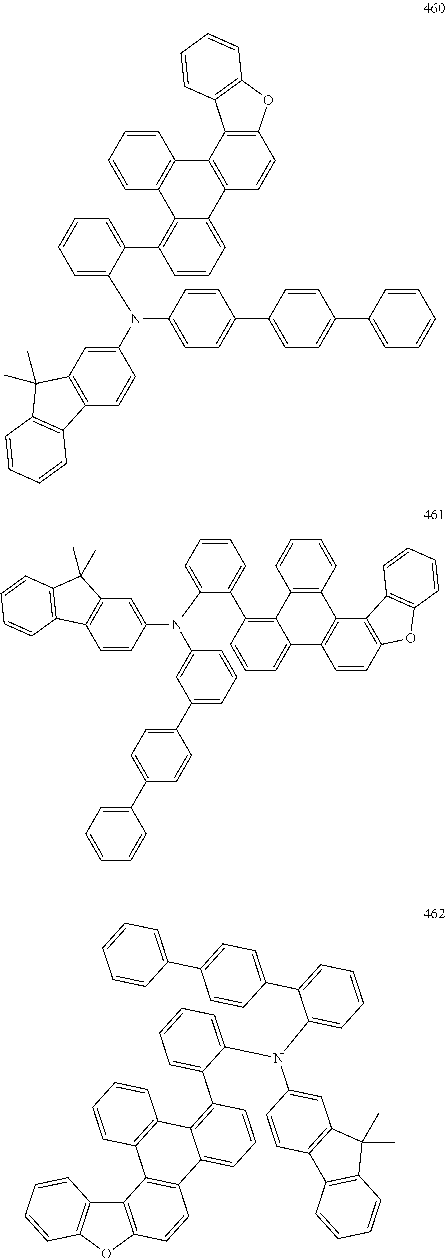

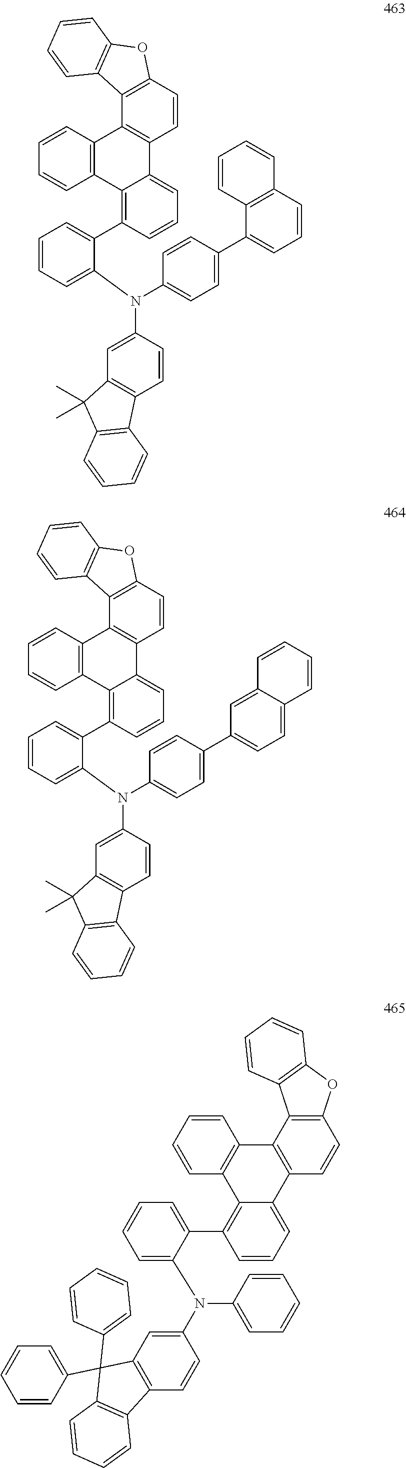

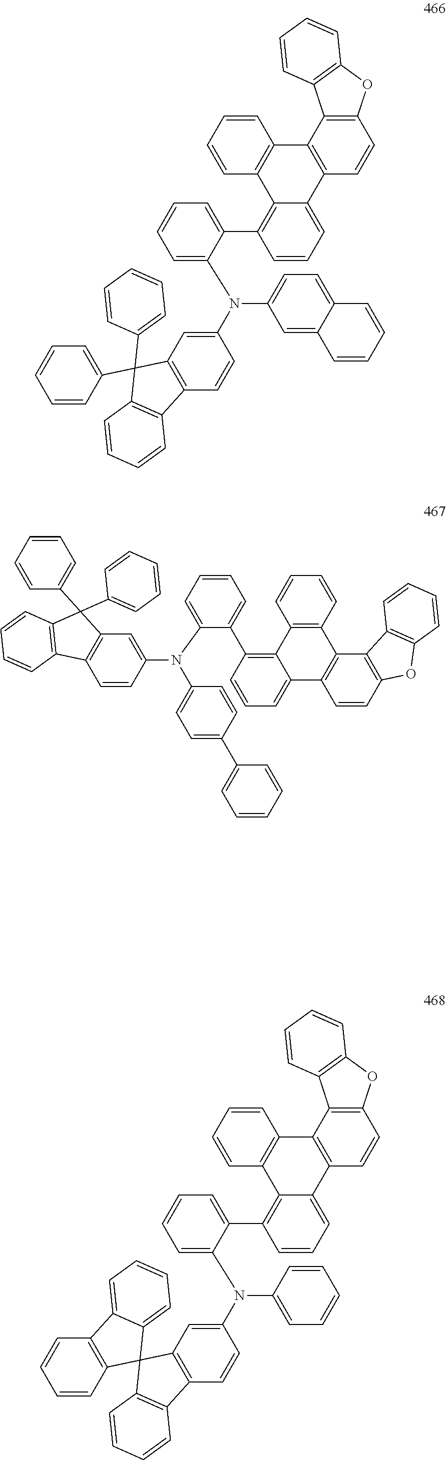 [SMILES: c1ccc(C2(c3ccccc3)c3ccccc3-c3ccc(N(c4ccc5ccccc5c4)c4ccccc4-c4cccc5c6ccc7oc8ccccc8c7c6c6ccccc6c45)cc32)cc1 ; c1ccc(-c2ccc(N(c3ccc4c(c3)C(c3ccccc3)(c3ccccc3)c3ccccc3-4)c3ccccc3-c3cccc4c5ccc6oc7ccccc7c6c5c5ccccc5c34)cc2)cc1 ; c1ccc(N(c2ccc3c(c2)C2(c4ccccc4-c4ccccc42)c2ccccc2-3)c2ccccc2-c2cccc3c4ccc5oc6ccccc6c5c4c4ccccc4c23)cc1]

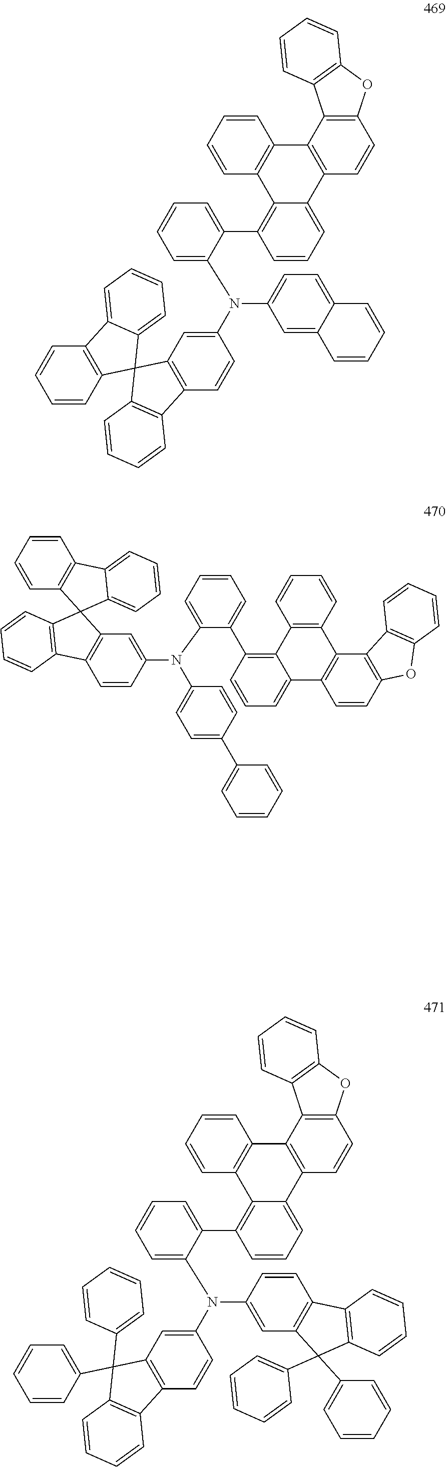 [SMILES: c1ccc(N(c2ccc3c(c2)C2(c4ccccc4-c4ccccc42)c2ccccc2-3)c2ccc3ccccc3c2)c(-c2cccc3c4ccc5oc6ccccc6c5c4c4ccccc4c23)c1 ; c1ccc(-c2ccc(N(c3ccc4c(c3)C3(c5ccccc5-c5ccccc53)c3ccccc3-4)c3ccccc3-c3cccc4c5ccc6oc7ccccc7c6c5c5ccccc5c34)cc2)cc1 ; c1ccc(C2(c3ccccc3)c3ccccc3-c3ccc(N(c4ccc5c(c4)C(c4ccccc4)(c4ccccc4)c4ccccc4-5)c4ccccc4-c4cccc5c6ccc7oc8ccccc8c7c6c6ccccc6c45)cc32)cc1]

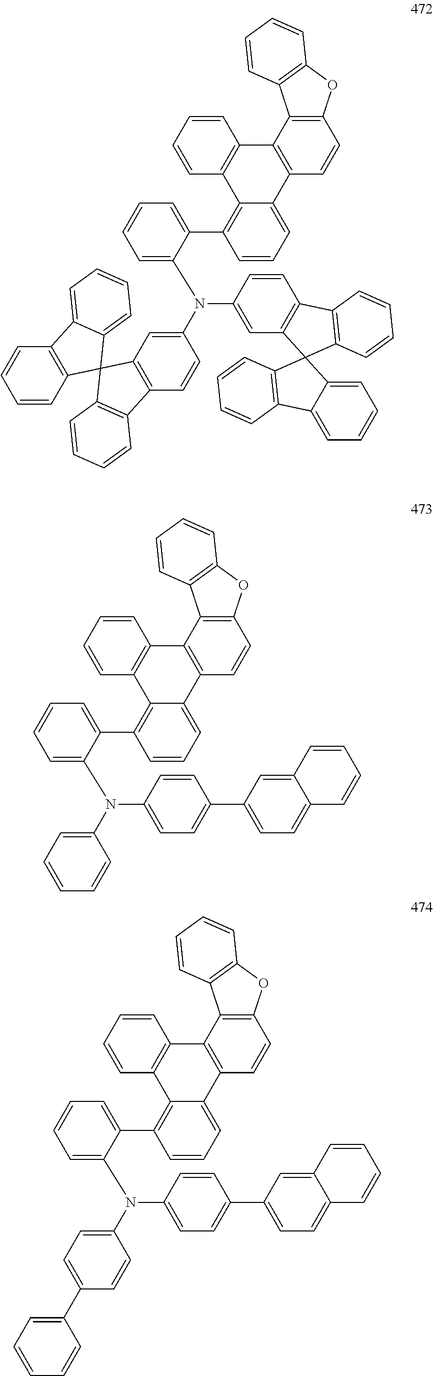 [SMILES: c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c4ccc5oc6ccccc6c5c4c4ccccc4c23)c1 ; c1ccc(N(c2ccc(-c3ccc4ccccc4c3)cc2)c2ccccc2-c2cccc3c4ccc5oc6ccccc6c5c4c4ccccc4c23)cc1 ; c1ccc(-c2ccc(N(c3ccc(-c4ccc5ccccc5c4)cc3)c3ccccc3-c3cccc4c5ccc6oc7ccccc7c6c5c5ccccc5c34)cc2)cc1]

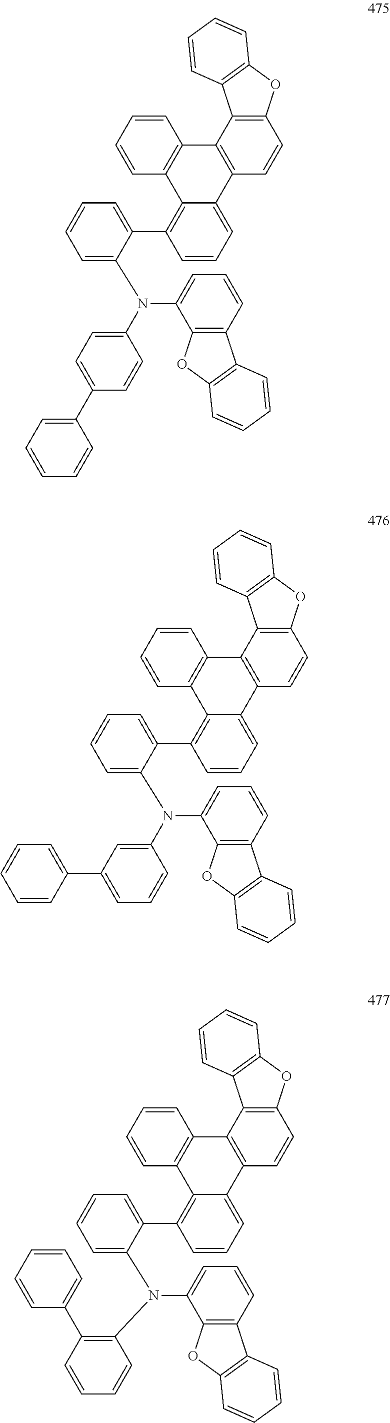 [SMILES: c1ccc(-c2ccc(N(c3ccccc3-c3cccc4c5ccc6oc7ccccc7c6c5c5ccccc5c34)c3cccc4c3oc3ccccc34)cc2)cc1 ; c1ccc(-c2cccc(N(c3ccccc3-c3cccc4c5ccc6oc7ccccc7c6c5c5ccccc5c34)c3cccc4c3oc3ccccc34)c2)cc1 ; c1ccc(-c2ccccc2N(c2ccccc2-c2cccc3c4ccc5oc6ccccc6c5c4c4ccccc4c23)c2cccc3c2oc2ccccc23)cc1]

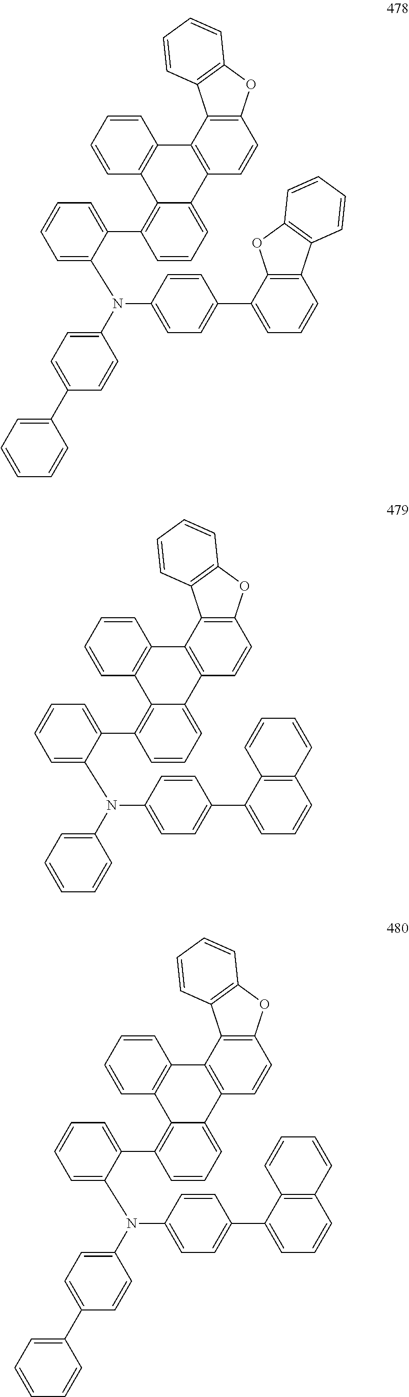 [SMILES: c1ccc(-c2ccc(N(c3ccc(-c4cccc5c4oc4ccccc45)cc3)c3ccccc3-c3cccc4c5ccc6oc7ccccc7c6c5c5ccccc5c34)cc2)cc1 ; c1ccc(N(c2ccc(-c3cccc4ccccc34)cc2)c2ccccc2-c2cccc3c4ccc5oc6ccccc6c5c4c4ccccc4c23)cc1 ; c1ccc(-c2ccc(N(c3ccc(-c4cccc5ccccc45)cc3)c3ccccc3-c3cccc4c5ccc6oc7ccccc7c6c5c5ccccc5c34)cc2)cc1]

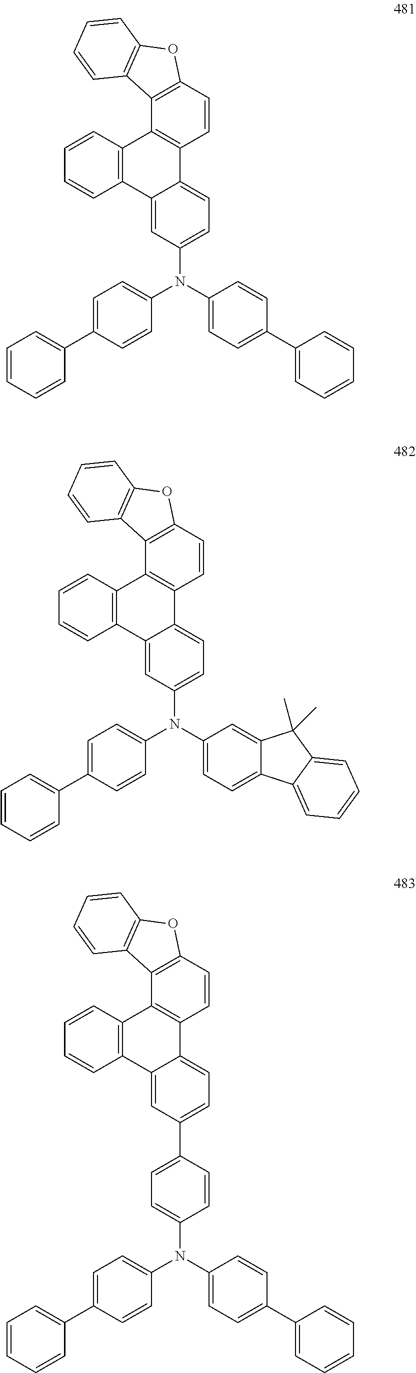 [SMILES: c1ccc(-c2ccc(N(c3ccc(-c4ccccc4)cc3)c3ccc4c(c3)c3ccccc3c3c4ccc4oc5ccccc5c43)cc2)cc1 ; CC1(C)c2ccccc2-c2ccc(N(c3ccc(-c4ccccc4)cc3)c3ccc4c(c3)c3ccccc3c3c4ccc4oc5ccccc5c43)cc21 ; c1ccc(-c2ccc(N(c3ccc(-c4ccccc4)cc3)c3ccc(-c4ccc5c(c4)c4ccccc4c4c5ccc5oc6ccccc6c54)cc3)cc2)cc1]

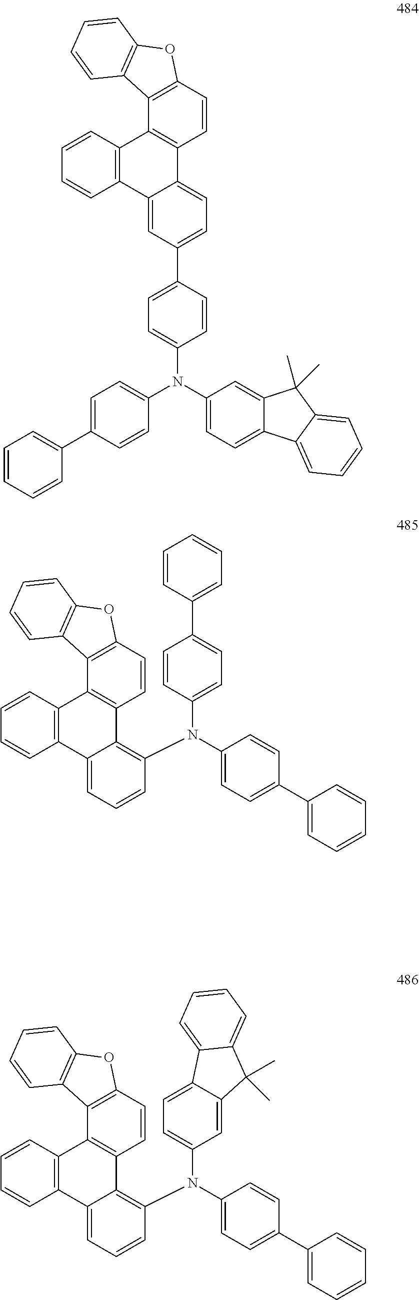 [SMILES: CC1(C)c2ccccc2-c2ccc(N(c3ccc(-c4ccccc4)cc3)c3ccc(-c4ccc5c(c4)c4ccccc4c4c5ccc5oc6ccccc6c54)cc3)cc21 ; c1ccc(-c2ccc(N(c3ccc(-c4ccccc4)cc3)c3cccc4c5ccccc5c5c(ccc6oc7ccccc7c65)c34)cc2)cc1 ; CC1(C)c2ccccc2-c2ccc(N(c3ccc(-c4ccccc4)cc3)c3cccc4c5ccccc5c5c(ccc6oc7ccccc7c65)c34)cc21]

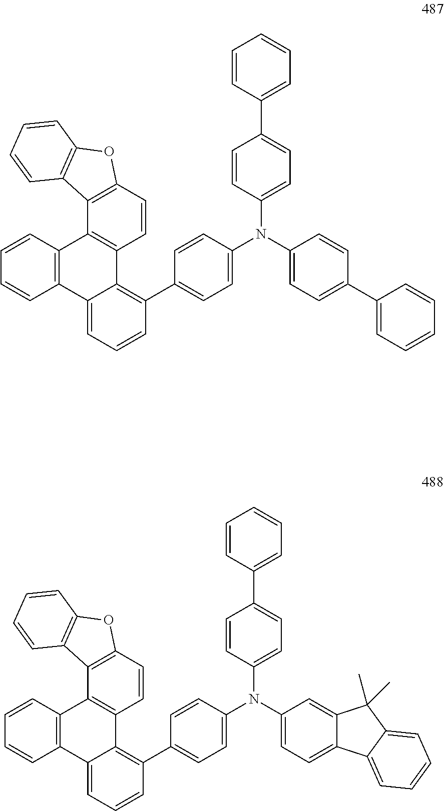 [SMILES: c1ccc(-c2ccc(N(c3ccc(-c4ccccc4)cc3)c3ccc(-c4cccc5c6ccccc6c6c(ccc7oc8ccccc8c76)c45)cc3)cc2)cc1 ; CC1(C)c2ccccc2-c2ccc(N(c3ccc(-c4ccccc4)cc3)c3ccc(-c4cccc5c6ccccc6c6c(ccc7oc8ccccc8c76)c45)cc3)cc21]

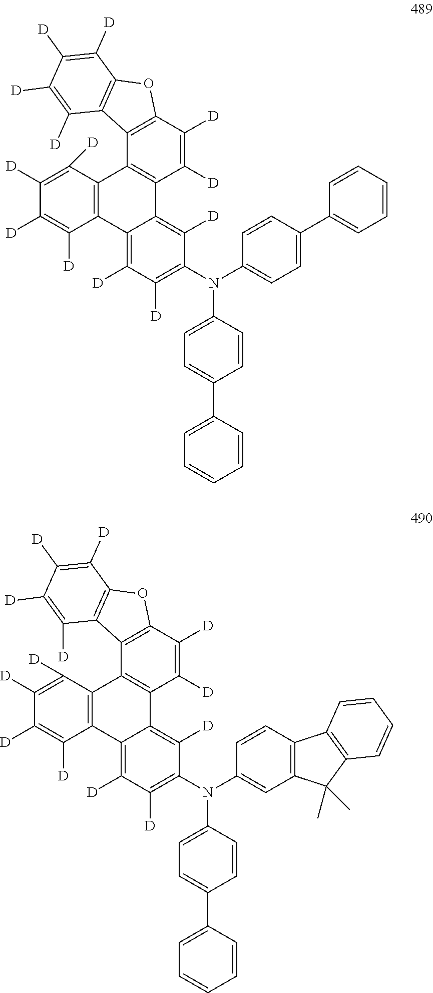 [SMILES: [2H]c1c([2H])c([2H])c2c(oc3c([2H])c([2H])c4c5c([2H])c(N(c6ccc(-c7ccccc7)cc6)c6ccc(-c7ccccc7)cc6)c([2H])c([2H])c5c5c([2H])c([2H])c([2H])c([2H])c5c4c32)c1[2H] ; [2H]c1c([2H])c([2H])c2c(oc3c([2H])c([2H])c4c5c([2H])c(N(c6ccc(-c7ccccc7)cc6)c6ccc7c(c6)C(C)(C)c6ccccc6-7)c([2H])c([2H])c5c5c([2H])c([2H])c([2H])c([2H])c5c4c32)c1[2H]]

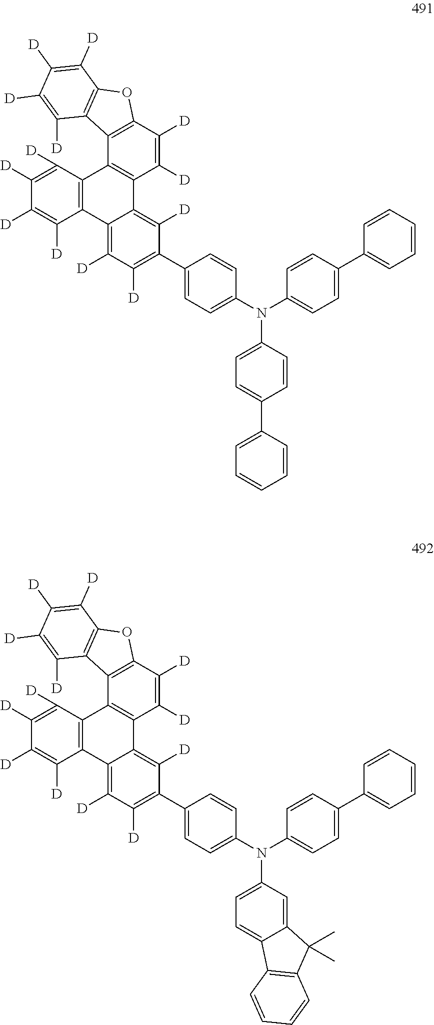 [SMILES: [2H]c1c([2H])c([2H])c2c(oc3c([2H])c([2H])c4c5c([2H])c(-c6ccc(N(c7ccc(-c8ccccc8)cc7)c7ccc(-c8ccccc8)cc7)cc6)c([2H])c([2H])c5c5c([2H])c([2H])c([2H])c([2H])c5c4c32)c1[2H] ; [2H]c1c([2H])c([2H])c2c(oc3c([2H])c([2H])c4c5c([2H])c(-c6ccc(N(c7ccc(-c8ccccc8)cc7)c7ccc8c(c7)C(C)(C)c7ccccc7-8)cc6)c([2H])c([2H])c5c5c([2H])c([2H])c([2H])c([2H])c5c4c32)c1[2H]]

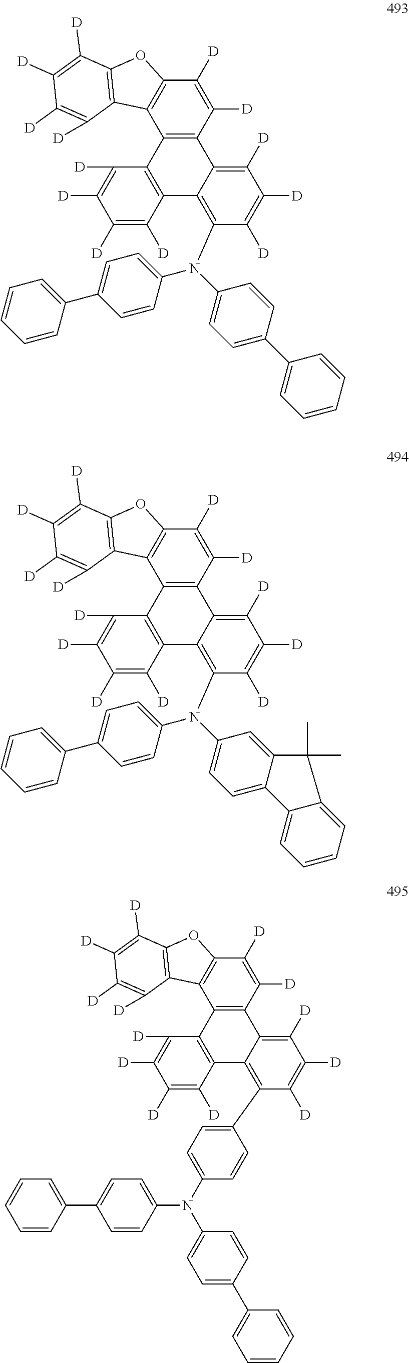 [SMILES: [2H]c1c([2H])c([2H])c2c(oc3c([2H])c([2H])c4c5c([2H])c([2H])c([2H])c(N(c6ccc(-c7ccccc7)cc6)c6ccc(-c7ccccc7)cc6)c5c5c([2H])c([2H])c([2H])c([2H])c5c4c32)c1[2H] ; [2H]c1c([2H])c([2H])c2c(oc3c([2H])c([2H])c4c5c([2H])c([2H])c([2H])c(N(c6ccc(-c7ccccc7)cc6)c6ccc7c(c6)C(C)(C)c6ccccc6-7)c5c5c([2H])c([2H])c([2H])c([2H])c5c4c32)c1[2H] ; [2H]c1c([2H])c([2H])c2c(oc3c([2H])c([2H])c4c5c([2H])c([2H])c([2H])c(-c6ccc(N(c7ccc(-c8ccccc8)cc7)c7ccc(-c8ccccc8)cc7)cc6)c5c5c([2H])c([2H])c([2H])c([2H])c5c4c32)c1[2H]]

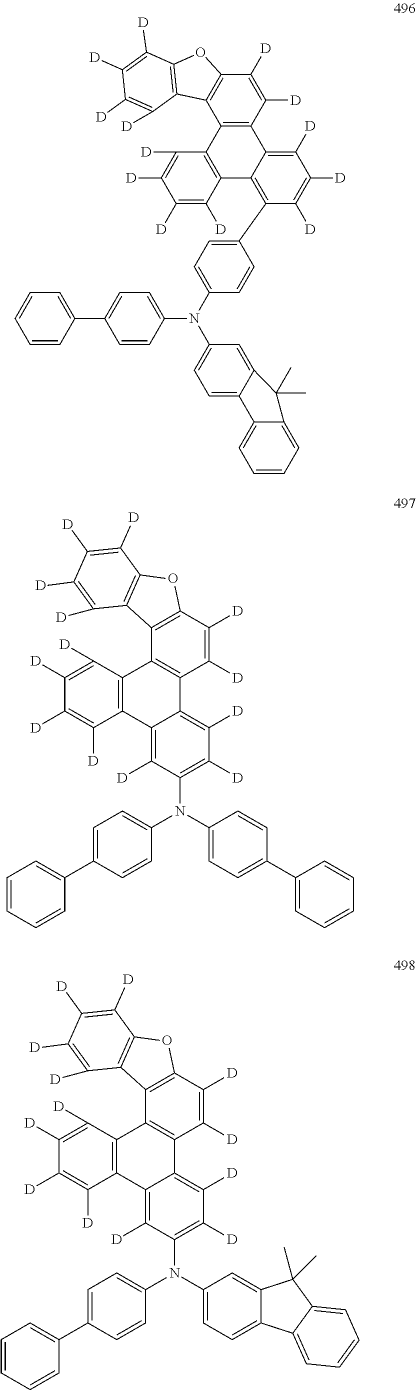

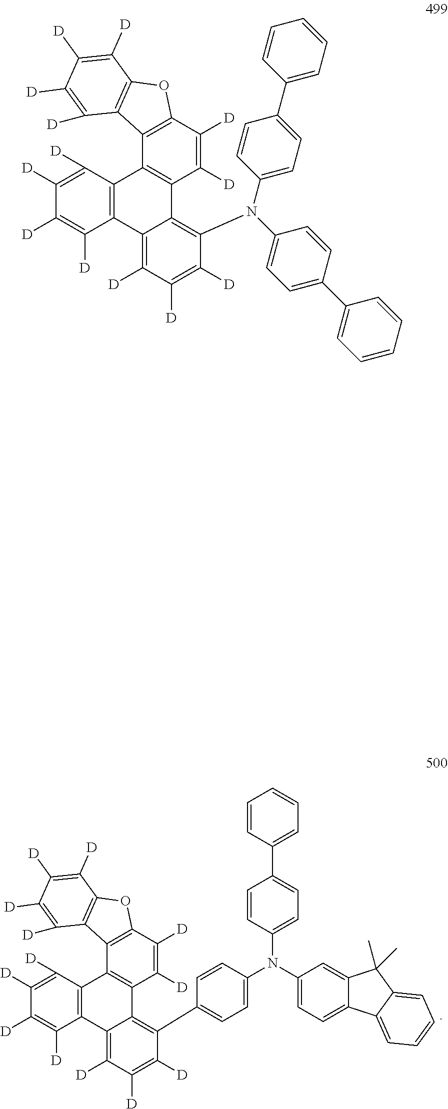 [SMILES: [2H]c1c([2H])c([2H])c2c(oc3c([2H])c([2H])c4c5c(N(c6ccc(-c7ccccc7)cc6)c6ccc(-c7ccccc7)cc6)c([2H])c([2H])c([2H])c5c5c([2H])c([2H])c([2H])c([2H])c5c4c32)c1[2H] ; [2H]c1c([2H])c([2H])c2c(oc3c([2H])c([2H])c4c5c(-c6ccc(N(c7ccc(-c8ccccc8)cc7)c7ccc8c(c7)C(C)(C)c7ccccc7-8)cc6)c([2H])c([2H])c([2H])c5c5c([2H])c([2H])c([2H])c([2H])c5c4c32)c1[2H]]

5. An organic light emitting device comprising,
a first electrode;
a second electrode provided to face the first electrode; and
one or more organic layers provided between the first electrode and the second electrode,
wherein at least one of the one or more organic layers comprises the heterocyclic compound according to claim 1.

6. The organic light emitting device according to claim 5, wherein the one or more organic layers comprises a hole transport layer, and the hole transport layer comprises the heterocyclic compound.

7. The organic light emitting device according to claim 5, wherein the one or more organic layers comprises an electron blocking layer, and the electron blocking layer comprises the heterocyclic compound.

8. The organic light emitting device according to claim 5, wherein the one or more organic layers comprises an electron injection layer, a hole injection layer, an electron transport layer or a hole blocking layer, and the electron transport layer, the hole injection layer, the electron injection layer or the hole blocking layer comprises the heterocyclic compound.

9. The organic light emitting device according to claim 5, wherein the organic light emitting device further comprises at least one selected from the group consisting of a light emitting layer, a hole injection layer, a hole transport layer, an electron injection layer, an electron transport layer, an electron blocking layer and a hole blocking layer.

10. A composition for organic layer of an organic light emitting device comprising the heterocyclic compound according to claim 1.

11. (canceled)
